SEQUENCE LISTING (4 sequences)

SEQ ID NO: 1 - protein, 15 aa, Artificial Sequence
  Synthetic peptide
DRRRRRRRRPIYALA

SEQ ID NO: 2 - protein, 18 aa, Artificial Sequence
  Synthetic peptide
GGGGGGRAEDSGNESEGE

SEQ ID NO: 3 - protein, 17 aa, Artificial Sequence
  Synthetic peptide
GGGGGGDRIIDSGLDSM

SEQ ID NO: 4 - protein, 14 aa, Artificial Sequence
  Synthetic peptide
GQEDATADDQYQQY

(19) United States
(12) Patent Application Publication
(10) Pub. No.: US 2022/0143183 A1
(43) Pub. Date: May 12, 2022

(54) PHOTOSWITCHABLE PROTACS AND SYNTHESIS AND USES THEREOF

(71) Applicant: New York University, New York, NY (US)

(72) Inventors: Dirk TRAUNER, New York, NY (US); Martin REYNDERS, Nordstrand (DE); Bryan MATSUURA, San Francisco, CA (US); Marleen BEROUTI, Munich (DE); Michele PAGANO, New York, NY (US)

(21) Appl. No.: 17/432,750

(22) PCT Filed: Feb. 24, 2020
(86) PCT No.: PCT/US20/19458

§ 371 Date: Aug. 20, 2021

Related U.S. Application Data
(60) Provisional application No. 62/809,587, filed on Feb. 23, 2019.

(51) Int. Cl. A61K 41/00; A61K 47/55; A61K 47/54; A61P 35/02

(52) U.S. Cl. CPC A61K 41/0042; A61P 35/02; A61K 47/545; A61K 47/55

(57) ABSTRACT

Provided are compounds, which may be referred to as PHOTACs (photoswitchable proteolysis targeting chimeras), and compositions, kits, and methods of making and using PHOTACs. PHOTACs have one or more E3 ligase ligand(s), one or more photoswitchable group(s), optionally, one or more linker(s), and one or more ligand(s) for a target protein. PHOTACs may be suitable for use in methods to treat diseases, such as, for example, cancer. PHOTACs may also be suitable for use in methods to induce selective degradation of a target protein.

### CROSS REFERENCE TO RELATED APPLICATIONS

This application claims priority to U.S. Provisional Application No. 62/809,587, filed on Feb. 23, 2019, the disclosure of which is incorporated herein.

### STATEMENT REGARDING FEDERALLY SPONSORED RESEARCH

This invention was made with government support under grant number CA076584 awarded by National Institutes of Health. The government has certain rights in the invention.

### BACKGROUND OF THE DISCLOSURE

Protein levels in cells result from a tightly controlled balance between de novo synthesis and degradation. A wide range of small molecules have been identified that interfere with these processes. Most of them do not address specific proteins as they broadly inhibit the machinery necessary for transcription, translation, trafficking, or degradation. In recent years proteolysis targeting chimeras (PROTACs) have emerged as a new principle of pharmacology. These bifunctional molecules combine a ligand for an E3 ligase with a second one that targets a protein of interest (POI) and catalyze its polyubiquitination and eventual proteasomal degradation. Both ends of the PROTACs are connected via a linker the exact nature of which needs to be carefully chosen to ensure efficacy, cell permeability, and biodistribution. Notably, PROTACs do merely inhibit the activity of their targets, like conventional drugs, but rather influence the levels at which these targets are present in a cell.

First generation PROTACs used peptides to recruit POIs to E3 ligases, but subsequent ones have relied on smaller and more cell-permeable synthetic ligands. These include hydroxyproline derivatives and molecules derived from thalidomide, which bind the von Hippel-Lindau protein (VHL) and cereblon (CRBN), respectively. VHL and CRBN are the substrate receptors of two cullin-RING ubiquitin ligase (CRL) complexes, namely CRL2^{VHL} and CRL4^{CRBN}. Proteins that have been successfully targeted for degradation through these ubiquitin ligases include the androgen and estrogen receptors, the BET family epigenetic readers BRD2-4, and FKPB12 and its fusion proteins. Soluble kinases, such as, for example, CDK9 and BCR-ABL, as well as receptor tyrosine kinases, such as, for example, EGFR and BTK, have also been amenable to this approach. Covering a broad spectrum from membrane proteins to nuclear hormone receptors, PROTACs have proven to be a highly versatile approach.

PROTACs do not merely inhibit the activity of their targets, like conventional drugs; rather, they decrease the levels of the targets by promoting their proteolysis. The transition from inhibition of proteins to their catalytic degradation enables the targeting of previously undruggable proteins. However, this mechanistic difference with conventional drugs also poses certain risks when applied systemically, since the POI is degraded and disappears with all of its functions in both cancer and normal cells. For instance, inhibition of BET bromodomains is tolerated, but a complete loss of BRD2 and BRD4 is lethal.

### SUMMARY OF THE DISCLOSURE

The present disclosure provides photoswitchable PROTACs (proteolysis targeting chimeras), also referred to herein as PHOTACs. The PHOTACs of the present disclosure may be referred to as compounds. Also provided are compositions and kits comprising PHOTACs of the present disclosure and methods of using the PHOTACs of the present disclosure.

In an aspect, the present disclosure provides PHOTACs. The PHOTACs of the present disclosure comprise one or more E3 ligase ligand(s), one or more photoswitchable group(s), optionally, one or more linker(s), and one or more ligand(s) for a target protein (which also may be referred herein to as a protein of interest).

In various examples, PHOTACs of the present disclosure have the following structure: A-PS-L-B, A-L-PS-B, PS-A-L-B, PS-A-L-B-PS, A-PS-L-PS-B, A-L-PS-L-B, PS-A-L-PS-B, or A-PS-L-B-PS, where A is an E3 ligase ligand, PS is a photoswitchable group, L is optional and is a linker, and B is a ligand for a target protein. In various examples, an E3 ligase ligand further comprises a photoswitchable group and/or a linker further comprises a photoswitchable group and/or ligand for a target protein further comprises a photoswitchable group. In such examples, a PHOTAC of the present disclosure may have the following structure: A′-L′-B′, where A′ is an E3 ligase ligand optionally comprising a photoswitchable group, L′ is an optional linker optionally comprising a photoswitchable group, and B′ is a ligand for a target protein optionally comprising a photoswitchable group. For example, a PHOTAC may have the following structure: PS-A-L-B-PS, where the photoswitchable group is part of the E3 ligase and another photoswitchable group is part of the ligand for a target protein. In an illustrative example, a PHOTAC having the structure PS-A-L-B may be:

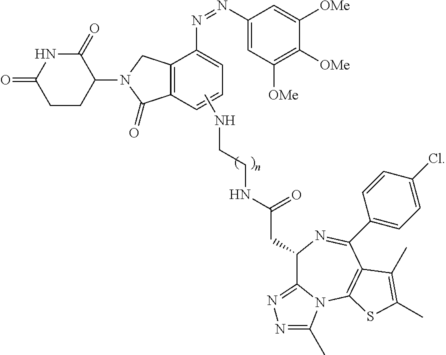

Various other illustrative examples of PHOTACs include, but are not limited to,

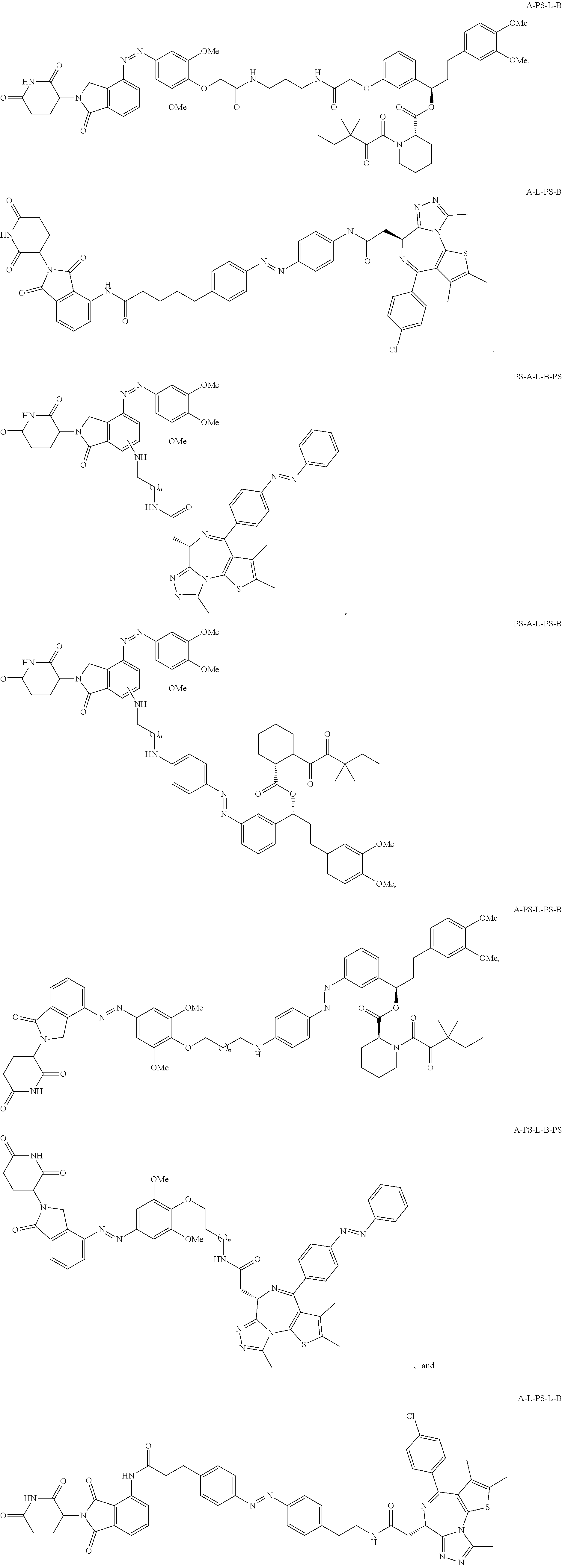

In various examples, an E3 ligase ligand, linker, and/or ligand for a target protein does not comprise a photoswitchable group.

In an aspect, the present disclosure provides compositions comprising PHOTACs of the present disclosure. The compositions may further comprise one or more pharmaceutically acceptable carrier(s).

In an aspect, the present disclosure provides methods of using one or more PHOTAC(s) or a composition comprising one or more PHOTAC(s). The PHOTACs of the present disclosure are suitable in methods to treat various cancers (e.g., leukemia, lung cancer, dermatological cancer, premalignant lesions of the upper digestive tract, malignancies of the prostate, malignancies of the brain, malignancies of the breast, and the like) and/or various other diseases (e.g., infectious diseases, inflammatory diseases, immune disorders, sleep disorders, neurodegenerative disorders, and the like) and methods to induce protein degradation. In various examples, one or more PHOTAC(s) of the present disclosure is used to treat such as, for example, cancer, other disease(s), or a combination thereof and/or induce selective degradation of a target protein. A method may be carried out in combination with one or more known therapy(ies).

### BRIEF DESCRIPTION OF THE FIGURES

For a fuller understanding of the nature and objects of the disclosure, reference should be made to the following detailed description taken in conjunction with the accompanying figures.

FIG. 1 depicts examples of PROTACs and PHOTACs. A) Schematic depiction of a PROTAC. Formation of a ternary complex between an E3-ligase, a PROTAC and a protein of interest (POI) leads to degradation of the POI. B) Chemical structures of PROTACs dBET1 and dFKBP-1. C) Schematic depiction of a PHOTAC. The molecules toggles between an inactive form (pentagon) and an active form (star) upon irradiation.

FIG. 2 depicts examples of structure and synthesis of PHOTACs. A) Members of the PHOTAC-I series targeting BRDs. B) Synthesis of PHOTAC-I-3 starting from lenalidomide. C) Members of the PHOTAC-II series targeting FKBP12.

FIG. 3 depicts photophysical properties, switching, and bistability of examples of PHOTACs. A) Switching of PHOTAC-I-3 between the trans isomer (left) and cis isomer (right). B) UV-VIS spectra PHOTAC-I-3 following irradiation with different wavelengths for 5 min. C) Fraction of trans PHOTAC-I-3 in the PSS. D) Thermal relaxation of cis PHOTAC-I-3 at 37° C. in DMSO. E) Reversible switching and photochemical stability of PHOTAC-I-3. F) Switching of PHOTAC-II-5 between the trans isomer (left) and cis isomer (right). G) UV-VIS spectra PHOTAC-II-5 following irradiation with different wavelengths for 5 min. H) Thermal relaxation of cis PHOTAC-II-5 at 37° C. in DMSO. I) Switching of PHOTAC-II-6 between the trans isomer (left) and cis isomer (right). J) UV-VIS spectra PHOTAC-II-6 following irradiation with different wavelengths for 5 min. K) Thermal relaxation of cis PHOTAC-II-6 at 37° C. in DMSO.

FIG. 4 depicts light-dependent viability of RS4;11 cells. A) Viability of RS4;11 acute lymphoblastic leukemia cells after treatment with PHOTAC-I-3 for 72 h in the dark or under pulsed (100 ms every 10 s) 390 nm, 477 nm or 545 nm irradiation. B) RS4;11 Viability after (+)-JQ1 treatment for 72 h in the dark or under pulsed (100 ms every 10 s) 390 nm irradiation. C) Viability of RS4;11 cells after 72 h in the dark or under pulsed (100 ms every 10 s) 390 nm irradiation.

FIG. 5 depicts optical control of BRD2-4 levels. A) Immunoblot analysis after treatment of RS4;11 cells with PHOTAC-I-3 for 4 h at different concentrations. Cells were either irradiated with 100 ms pulses of 390 nm light every 10 s (left) or kept in the dark (right). B) Time course of BRD2-4 degradation, c-MYC levels and PARP1 cleavage assayed by immunoblotting. RS4;11 cells were treated with PHOTAC-I-3 (1 μM) and collected at the indicated time points. PHOTAC-I-3 has no effect on BRD2-4 levels in the dark over several hours. C) Immunoblot of a rescue experiment demonstrating the reversibility of degradation promoted by PHOTAC-I-3 through thermal relaxation (left) or optical inactivation by 525 nm pulsed irradiation (right, 100 ms every 10 s). D) Color-dosing: Wavelength dependence of BRD2/4 degradation promoted by 300 nM PHOTAC-I-3. E) Immunoblot analysis after treatment of RS4;11 cells with PHOTAC-I-3 and combinations with lenalidomide or (+)-JQ1 for 4 h to confirm a cereblon-based mechanism. Cells were either irradiated with 100 ms pulses of 390 nm light every 10 s (left) or kept in the dark (right). F) Optical degradation of BRD4 with the thalidomide derivative PHOTAC-I-10. Immunoblot analysis of RS4;11 cells after treatment with PHOTAC-I-10 for 4 h at different concentrations which were either irradiated with 100 ms pulses of 390 nm light every 10 s (left) or kept in the dark (right).

FIG. 6 depicts optical control of FKBP12 degradation. A) Immunoblot analysis of FKBP12 after treatment of RS4;11 cells with PHOTAC-II-5 for 4 h at different concentrations. Cells were either irradiated with pulses of 390 nm light (left, 100 ms every 10 s) or kept in the dark (right). B) Time course of FKBP12 degradation visualized by immunoblotting. RS4;11 cells were treated with PHOTAC-II-5 (100 nM) and collected at the indicated time points. C) Immunoblot analysis of FKBP12 after treatment of RS4;11 cells with PHOTAC-II-6 for 4 h at different concentrations. Cells were either irradiated with pulses of 390 nm light (left, 100 ms every 10 s) or kept in the dark (right). D) Time course of FKBP12 degradation visualized by immunoblotting. RS4;11 cells were treated with PHOTAC-II-6 (100 nM) and collected at the indicated time points.

FIG. 7 depicts UV-Vis characterization. A) UV-VIS spectra of PHOTACs-I and PHOTACs-II following irradiation with the indicated wavelengths for 5 min. B) Separated UV-VIS spectra of (E)- and (Z)-PHOTAC-I-3 as obtained from the LCMS and normalized at the isosbestic point.

FIG. 8 depicts thermal relaxation data. A) Thermal relaxation of (Z)-PHOTACs in DMSO. B) Thermal relaxation of (Z)-PHOTACs in DMSO-PBS mixtures.

FIG. 9 depicts viability of RS4;11 acute lymphoblastic leukemia cells after treatment with PHOTACs-I for 72 h in the dark or under pulsed (100 ms every 10 s) 390 nm irradiation.

FIG. 10 depicts an immunoblot analysis of PHOTAC-I-3 A) Immunoblot analysis after treatment of MB-MDA-231 cells with PHOTAC-I-3 for 18 h at different concentrations. Cells were either irradiated with 100 ms pulses of 390 nm light every 10 s (left) or kept the dark (right). B) Immunoblot analysis after treatment of MB-MDA-468 cells with PHOTAC-I-3 for 18 h at different concentrations. Cells were either irradiated with 100 ms pulses of 390 nm light every 10 s (left) or kept the dark (right). (MWM, molecular weight marker). C) Immunoblot of BRD4 degradation in RS4;11 cells, promoted by PHOTAC-I-3 (300 nM) after 1 minute of irradiation (100 ms every 10 s), highlighting sustained degradation at the applied dosage.

FIG. 11 depicts an immunoblot analysis of BRD4 after treatment of RS4;11 cells with PHOTACs. A) PHOTAC-I-6, B) PHOTAC-I-9, C) PHOTAC-I-11, D) PHOTAC-I-12 or E) PHOTAC-I-13 for 4 h at different concentrations. Cells were either irradiated with 100 ms pulses of 390 nm light every 10 s (left) or kept the dark (right). (MWM, molecular weight marker).

FIG. 12 depicts a model of the photoswitch-cereblon interaction. Structure model of an (E)-(A) or (Z)-(B) azobenzene bound to cereblon. The model derived from the crystal structure of lenalidomide bound to cereblon (PDB: 4CI2), showing the clash between the (E)-azobenzene and cereblon, whereas the (Z)-isomer is accommodated by the binding pocket. Models were created using Schrödinger Maestro 11.9. (C) Overlay of both structures.

FIG. 13 depicts CRBN knockdown control. A) Immunoblot of BRD4 degradation after 18 h of PHOTAC-I-3 treatment in the dark or with 390 nm pulsed irradiation (100 ms every 10 s) in MB-MDA-231 cells, pre-treated with CRBN siRNA or non-targeting (NT) siRNA. B) mRNA expression levels of CRBN in MB-MDA-231 cells, treated with CRBN siRNA or non-targeting (NT) siRNA. C) Immunoblot of BRD3 degradation after 18 h of PHOTAC-I-3 treatment in the dark or with 390 nm pulsed irradiation (100 ms every 10 s) in MB-MDA-231 cells, pre-treated with CRBN siRNA or non-targeting (NT) siRNA.

FIG. 14 depicts Me-PHOTAC-I-3 control. A) Structure of inactive control Me-PHOTAC-I-3. B) Immunoblot of BRD4 levels after 4 h of Me-PHOTAC-I-3 treatment in RS4;11 cells in the dark or with 390 nm pulsed irradiation (100 ms every 10 s).

FIG. 15 depicts FKBP12 immunoblots in RS4;11 cells. A) Immunoblot of a rescue experiment demonstrating the reversibility of FKBP12 degradation promoted by PHOTAC-II-5 through thermal relaxation (left) or optical inactivation by 525 nm pulsed irradiation (right, 100 ms every 10 s). B) Degradation of FKBP12. Immunoblot analysis of FKBP12 after treatment of RS4;11 cells with PHOTAC-II-1 for 4 h at different concentrations. Cells were either irradiated with 100 ms pulses of 390 nm light every 10 s (left) or kept the dark (right). C) Time course of FKBP12 degradation visualized by immunoblotting. RS4;11 cells were treated with PHOTAC-II-1 (300 nM) and collected at the indicated time points, showing slow, but sustained FKBP12 degradation over time when irradiated with 390 nm light (left, 100 ms every 10 s), but not when kept in the dark (right).

FIG. 16 depicts an immunoblot analysis of FKBP12 after treatment of RS4;11 cells. A) PHOTAC-II-2, B) PHOTAC-II-3 or C) PHOTAC-II-4 for 4 h at different concentrations. Cells were either irradiated with pulses of 390 nm light (right, 100 ms every 10 s) or kept the dark (left).

FIG. 17 depicts photophysical properties, and switching of PHOTACs KR85, KR93, KR94, KR129 and KR114.

FIG. 18 depicts viability of RS4;11 acute lymphoblastic leukemia cells after treatment with the indicated PHOTACs for 72 h in the dark or under pulsed (100 ms every 10 s) irradiation using the indicated wavelengths.

FIG. 19 depicts an immunoblot analysis of RS4;11 cells after treatment with the indicated PHOTACs for 4 h. Cells were either irradiated with 100 ms pulses of the indicated wavelengths every 10 s (left) or kept the dark (right). MLN (MLN4924).

FIG. 20 depicts viability of RS4;11 acute lymphoblastic leukemia cells after treatment with the indicated PHOTACs for 72 h in the dark or under pulsed (100 ms every 10 s) irradiation using the indicated wavelengths.

FIG. 21 depicts an immunoblot analysis of RS4;11 cells after treatment with PHOTACs MR-MB-142, MR-MB-145, MR-MB-148, MR-MB-137, MR-MB-200 and MR-MB-201 for 4 h and after treatment with PHOTACs MR-MB-139 for 12 h. Cells were either irradiated with 100 ms pulses of the indicated wavelengths every 10 s (left) or kept the dark (right). MLN (MLN4924).

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, and process step may be made without departing from the scope of the disclosure.

Ranges of values are disclosed herein. The ranges set out an example of a lower limit value and an example of an upper limit value. Unless otherwise stated, the ranges include all values to the magnitude of the smallest value (either lower limit value or upper limit value) and ranges between the values of the stated range.

As used herein, the terms “including,” “containing,” and “comprising” are used in their open, non-limiting sense.

The articles “a” and “an” are used in this disclosure to refer to one or more than one (i.e., to at least one) of the grammatical object of the article. By way of example, “an element” means one element or more than one element.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. In an example, about refers to ±1%, ±2%, ±3%, ±4%, ±5%. ±6%, ±7%, ±8%, ±9%, ±10%, ±15%, or ±20%

As used herein, unless otherwise stated or indicated, “s” refers to second(s), “min” refers to minute(s), and “h” refers to hour(s).

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include, but are not limited to:

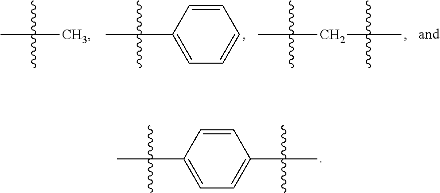

As used herein, unless otherwise indicated, the term “alkyl group” or “alkyl” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is a C₁ to C₁₅ alkyl group, including all integer numbers of carbons and ranges of numbers of carbons therebetween. The alkyl group may be unsubstituted or substituted with one or more substituent(s). The substituents can themselves be optionally substituted. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “aryl group” or “aryl” refers to C₅ to C₂₄ aryl groups (aromatic or partially aromatic carbocyclic groups), including all integer numbers of carbons and ranges of numbers of carbons therebetween. An aryl group can also be referred to as an aromatic group. The aryl groups may be polyaryl groups such as, for example, fused ring or biaryl groups. The aryl group may be unsubstituted or substituted with one or more substituent(s). The substituents can themselves be optionally substituted. Examples of substituents include, but are not limited to, substituents such as, for example, halogens (—F, —Cl, —Br, and —I), aliphatic groups (e.g., alkenes, alkynes, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), and the like.

As used herein, the term “heteroaryl group” or “heteroaryl” refers a monovalent, divalent, or polyvalent aromatic group comprising 5 to 18 ring atoms, containing one or more ring heteroatom(s) selected from N, O, or S, the remaining ring atoms are C. Heteroaryl groups may be polycyclic (e.g., bicyclic) or monocyclic. A heteroaryl group may be substituted with one or more substituent(s). The substituents can themselves be optionally substituted. Examples of substituents include, but are not limited to, substituents such as, for example, halogens (—F, —Cl, —Br, and —I), aliphatic groups (e.g., alkenes, alkynes, and the like), aryl groups, alkoxides, carboxylates, carboxylic acids, ether groups, and the like, and combinations thereof. Examples of heteroaryl groups include, but are not limited to, benzothiophene, furyl, thienyl, pyrrolyl, pyridyl, pyrazinyl, pyrazolyl, pyridazinyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, benzoimidazolyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and the like, and derivatives thereof. Heteroaryl groups containing two fused rings may have an unsaturated or partially saturated ring fused with a fully saturated ring.

The present disclosure provides photoswitchable PROTACs (proteolysis targeting chimeras), also referred to herein as PHOTACs. The PHOTACs of the present disclosure may be referred to as compounds. Also provided are compositions and kits comprising PHOTACs of the present disclosure and methods of using PHOTACs of the present disclosure.

In an aspect, the present disclosure provides PHOTACs. The PHOTACs of the present disclosure comprise one or more E3 ligase ligand(s), one or more photoswitchable group(s), optionally, one or more linker(s), and one or more ligand(s) for a target protein (a target protein is also referred to herein a protein of interest).

In various examples, PHOTACs of the present disclosure have the following structure: A-PS-L-B, A-L-PS-B, PS-A-L-B, PS-A-L-B-PS, A-PS-L-PS-B, A-L-PS-L-B, PS-A-L-PS-B, or A-PS-L-B-PS, where A is an E3 ligase ligand, PS is a photoswitchable group, L is optional and is a linker, and B is a ligand for a target protein. In various examples, an E3 ligase ligand further comprises a photoswitchable group or a linker further comprises a photoswitchable group or ligand for a target protein further comprises a photoswitchable group. In such examples, a PHOTAC of the present disclosure may have the following structure: A′-L′-B′, where A′ is an E3 ligase ligand optionally comprising a photoswitchable group, L′ is an optional linker optionally comprising a photoswitchable group, and B′ is a ligand for a target protein optionally comprising a photoswitchable group. In various examples, a PHOTAC has the following structure: PS-A-L-B-PS, where the photoswitchable group is part of the E3 ligase and another photoswitchable group is part of the ligand for a target protein. In an illustrative example, a PHOTAC having the structure PS-A-L-B may be:

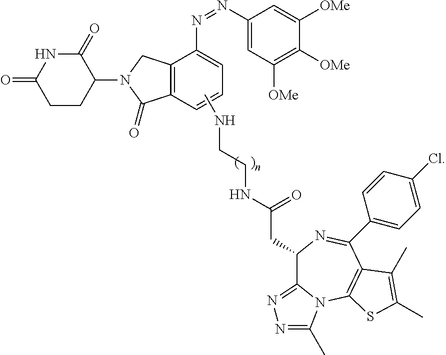

Various other illustrative examples of PHOTACs include, but are not limited to,

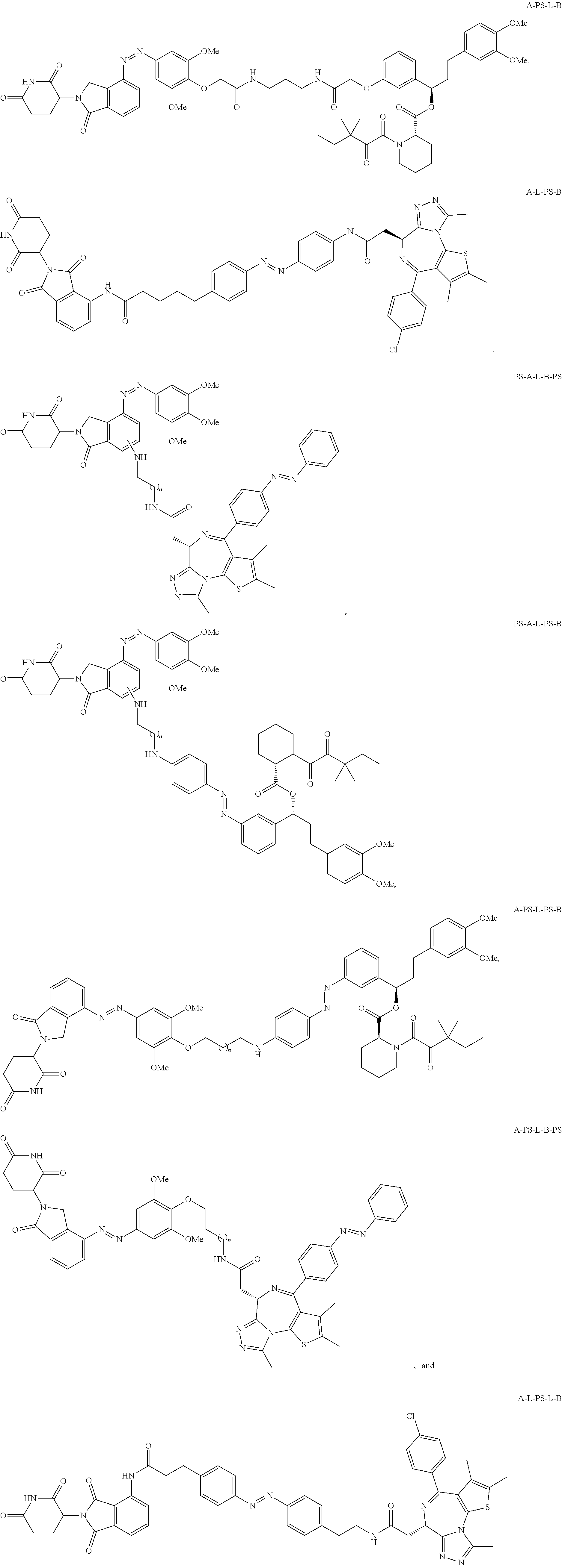

In various examples, an E3 ligase ligand, linker, and/or ligand for a target protein does not comprise a photoswitchable group.

In various examples, the E3 ligase ligand is a ligand for VHL, CRBN, RNF114, MDM2, DCAF15, DCAF16, and/or SCF. Non-limiting examples of E3 ligase ligands include:

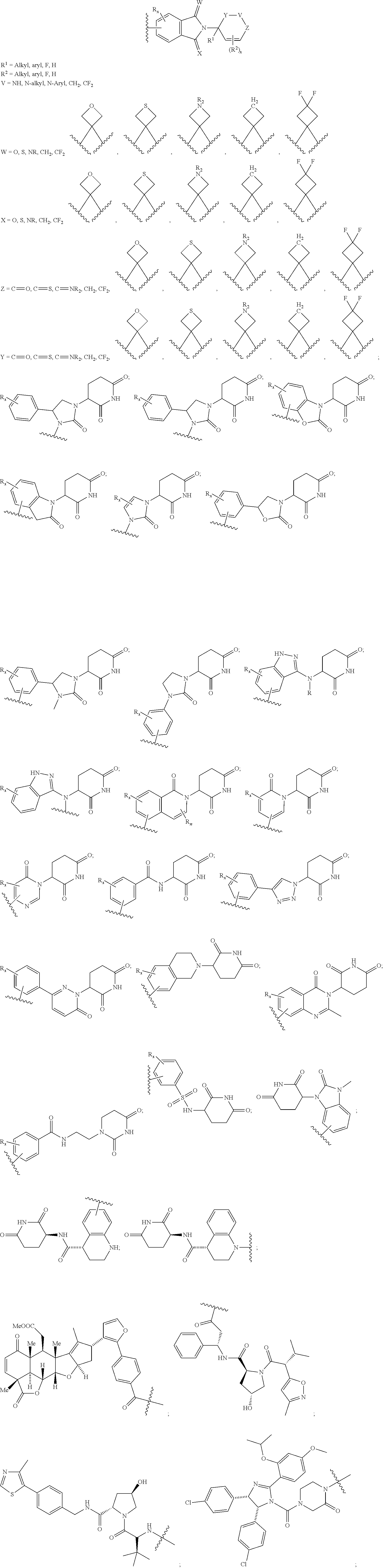

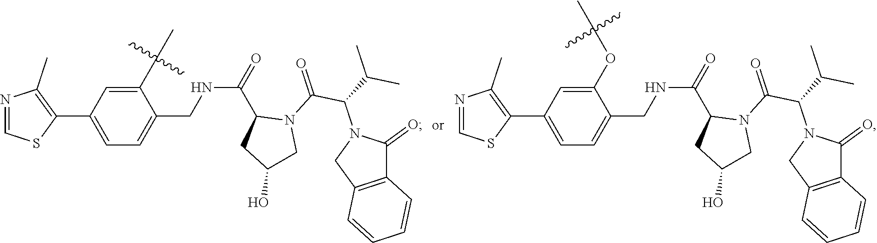

where R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂; H2N-(D-R)₈-PIYALA- (SEQ ID NO:1), GGGGGGRAEDS*GNES*EGE-COOH (SEQ ID NO:2) where * is a phosphorylated serine, GGGGGGDRIIDS*GLDS*M-COOH (SEQ ID NO:3) where * is a phosphorylated serine, and a photoswitchable group, and x is 0, 1, 2, 3, 4, or 5.

A photoswitchable group is a group that isomerizes upon exposure to a first wavelength of electromagnetic radiation. The isomerization may be reversible upon exposure to a second wavelength of electromagnetic radiation. A photoswitchable group may also isomerize via relaxation (e.g., after a period of time the photoswitch will isomerize back to a more thermodynamically favorable state). In various examples, a photoswitchable group in a thermodynamically favorable state is referred to as “relaxed” (e.g., it is at a thermodynamic minimum). In various examples, a photoswitchable group that has been exposed to electromagnetic radiation such that it is not in a thermodynamically favorable state is referred to as “unstable.” A photoswitchable group may isomerize from an unstable state to a relaxed state over a period of time or via exposure to electromagnetic radiation, and a photoswitchable group may isomerize from a relaxed state to an unstable state via exposure to electromagnetic radiation. In various examples, the period of time over which a photoswitchable group relaxes is tuned by the substituents and/or structure of the photoswitchable group. In various other examples, the isomerization of a photoswitchable group in a PHOTAC is referred to as “activated” (e.g., when the PHOTAC can bind both an E3 ligase and a target protein (e.g., protein of interest)) or “deactivated” (e.g., when the PHOTAC can bind only an E3 ligase or a target protein (e.g., protein of interest)).

In various examples, an azobenzene-based photoswitchable group in the trans conformation (i.e., E conformation) is isomerized to the cis conformation (i.e., Z conformation) upon exposure to electromagnetic radiation (e.g., light) having a wavelength of 375-405 nm (e.g., 390 nm), including all nm values and ranges therebetween, and the cis conformation is isomerized to the trans conformation upon exposure to electromagnetic radiation (e.g., light) having a wavelength of 485-515 nm (e.g., 500 nm), including every nm value and range therebetween.

In some examples, the photoswitchable group is part of the E3 ligase ligand. A non-limiting example of a photoswitchable group that is part of the E3 ligase ligand is an E3 ligase ligand that is thalidomide-based and the photoswitchable group is diazocine-based (e.g.,

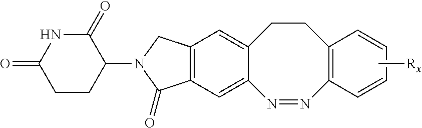

and the like),
azobenzene-based (e.g.,

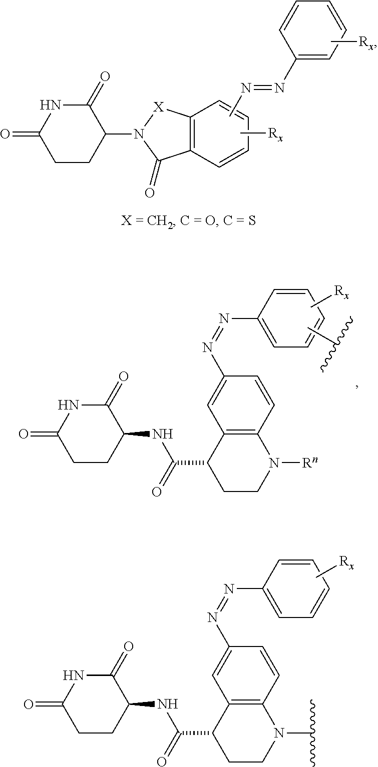

and the like), or diaryl ethene-based (e.g.,

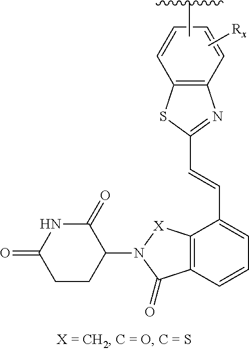

and the like).
Also included are hemithioindigo-based groups and the like.

Non-limiting examples of photoswitchable groups include:

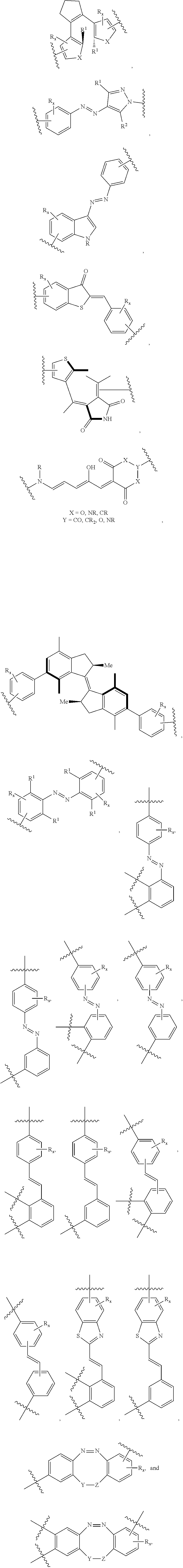

where R′ is F, Cl, Me, or OMe; R is H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂; x is 0, 1, 2, 3, 4, or 5; X is methylene, C═O, or C═S; and Y is methylene, O, or S; and Z is methylene, O, or S. In various examples, the photoswitchable group(s) can be (is) isomerized using electromagnetic radiation from 300-1500 nm (e.g., 350-650 nm for most azobenzene-based photoswitches). In various examples, the photoswitchable group can be (is) isomerized using two photon activation methods.

In various examples, a phenyl group of a photoswitchable group (e.g., an azobenzene-based photoswitchable group) has various substituents. Non-limiting examples of a substituted phenyl group of a photoswitchable group (e.g., an azobenzene-based photoswitchable group) include:

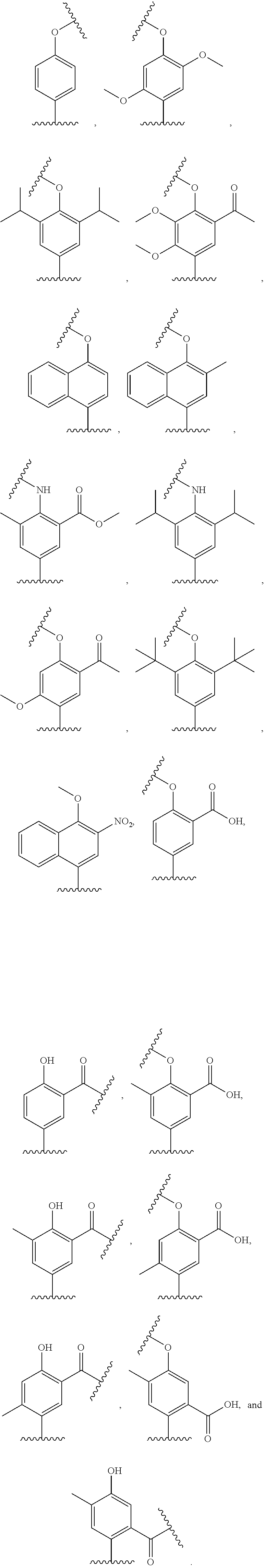

In various examples, a PHOTACs of the present disclosure comprises one or more linker(s). Various linkers are known in the art. Non-limiting examples of linkers include:

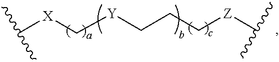

where Y is methylene or O, X and Z are independently methylene, O, NH, S,

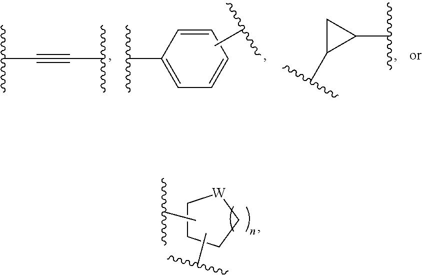

where W is methylene, NH, O, or S and n is greater than or equal to 0 (e.g., 0, 1, 2, 3, or 4); and a is 0-10, including every integer value and range therebetween, b is 0-10, including every integer value and range therebetween, and c is 0-10, including every integer value and range therebetween;

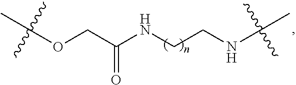

where n is 2, 3, 4, or 5; groups formed from polyethylene glycol groups (e.g.,

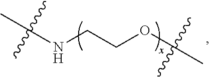

where x is 1-4;

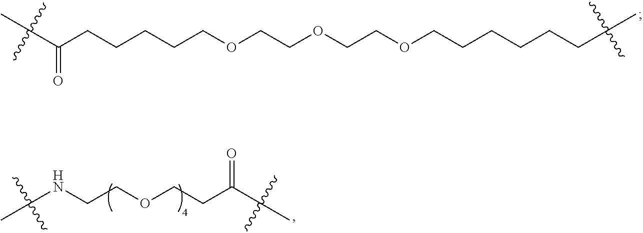

and the like); alkyl linkers (e.g.,

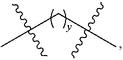

where y is 1-20;

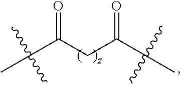

where z is 1-20; and the like); and peptide-based linkers (e.g., -SGSG- and the like).

In various examples, the following structures are part of the linker and/or part of a ligand (e.g., a target ligand, such as, for example, a ligand for a target protein):

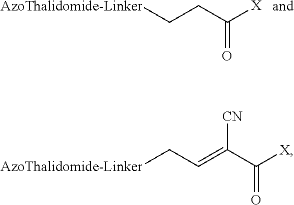

where X is the ligand for the target protein.

PHOTACs of the present disclosure comprise ligands for target proteins. Non-limiting examples of target proteins include EGFR, KRAS, and the like. Additional non-limiting examples of target proteins include Tau-protein, microbial DHFR, thymidylate synthase (TS), mammalian dihydrofolate reductase (DHFR), and glycinamide ribonucleotide formyltransferase (GARFT) and other proteins of the folate metabolism, HSP70, HSP90, ET proteins (e.g., BRD2,3,4, and the like), BTK, ABL, ALK, MET, MDM2, Tau, HDAC, FKBP12, angiogenesis inhibitors, human lysine methyltransferase inhibitors, aryl hydrocarbon receptors, estrogen receptors, androgen receptors, glucocorticoid receptors, transcription factors (e.g., SMARCA2/4, TRIM24, and the like), tyrosine kinases (e.g., EGFRs, FGFRs, and the like) serine/threonine kinases (e.g., Raf kinases, CaM kinases, AKT1, AKT2, AKT3, Aurora A, Aurora B, Aurora C, CHK1, CHK2, ERK1, ERK2, ERK5, MAP kinases, and the like), cyclin dependent kinases (e.g., CDK1-CDK11, such as, for example, CDK4/6, CDK9, and the like), B7.1 and B7, TINFR1m, TNFR2, NADPH oxidase, BcllBax and other partners in the apotosis pathway, C5a receptor, HMG CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, Gq, histamine receptors, 5-lipoxygenase, tryptase serine protease, thymidylate synthase, purine nucleoside phosphorylase, GAPDH trypanosomal, glycogen phosphorylase, carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CDS, IL-2 receptor, IL-1 receptor, TNF-alphaR, ICAM1, Cat+channels, VCAM, VLA-4 integrin, selectins, CD40/CD40L, newokinins and receptors, inosine monophosphate dehydrogenase, p38 MAP Kinase, Ras/Raf/ME/ERK pathway, interleukin-1 converting enzyme, caspase, HCV, NS3 protease, HCV NS3, RNA helicase, glycinamide ribonucleotide formyl transferase, rhinovirus 3C protease, herpes simplex virus-1 (HSV I), protease, cytomegalovirus (CMV) protease, poly (ADP ribose) polymerase, cyclin dependent kinases, vascular endothelial growth factor, c-Kit, TGFB activated kinase 1, mammalian target of rapamycin, SHP2, androgen receptor, oxytocin receptor, microsomal transfer protein inhibitor, bile acid transport inhibitor, 5 alpha reductase inhibitors, angiotensin 11, glycine receptor, noradrenaline reuptake receptor, estrogen receptor, estrogen related receptors, focal adhesion kinase, Src, endothelin receptors, neuropeptide Y and receptor, adenosine receptors, adenosine kinase and AMP deaminase, purinergic receptors (e.g., P2Y1, P2Y2, P2Y4, P2Y6, P2X1 7, and the like), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipase A2, EGF receptor tyrosine kinase, and the like, and combinations thereof. Additional protein targets include, for example, ecdysone 20-monooxygenase, ion channel of the GABA gated chloride channel, acetylcholinesterase, voltage sensitive sodium channel protein, calcium release channel, chloride channels, and the like, and combinations thereof. Still further target proteins include, but are not limited to, acetyl-CoA carboxylase, adenylosuccinate synthetase, protoporphyrinogen oxidase, enolpyruvylshikimate-phosphate synthase, and the like, and combinations thereof. A ligand for a target protein or target proteins may target a single protein or a plurality of proteins (e.g., one or more of the aforementioned target proteins).

Non-limiting examples of ligands that target proteins include:

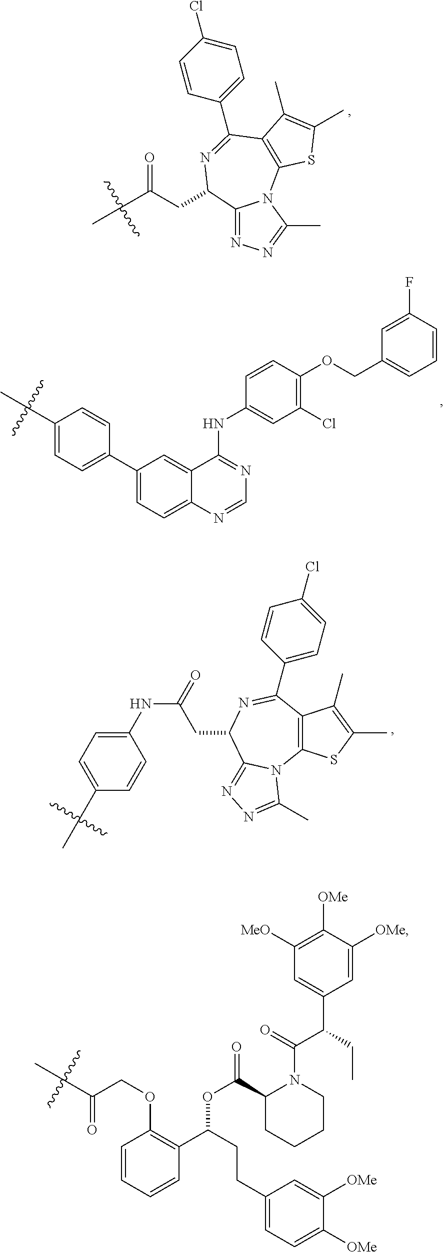

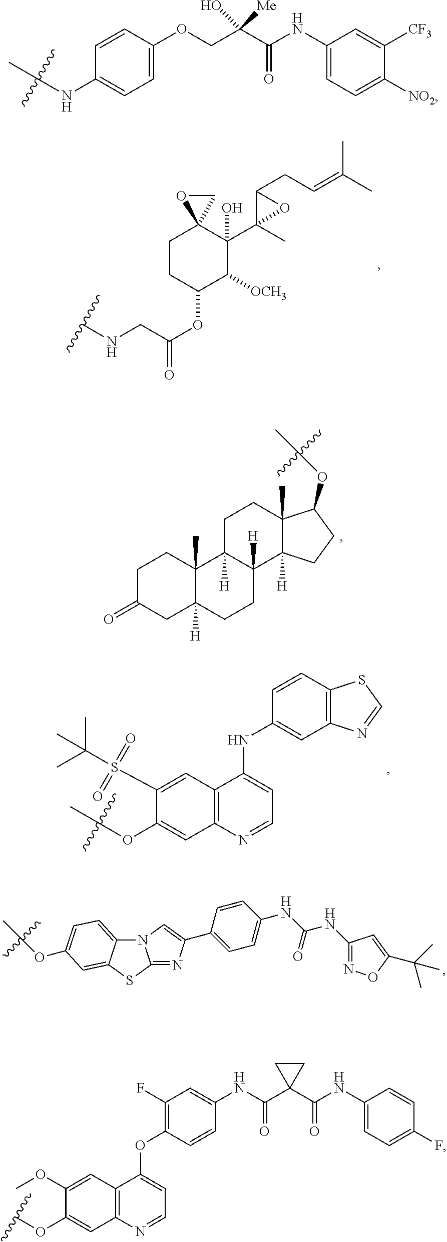

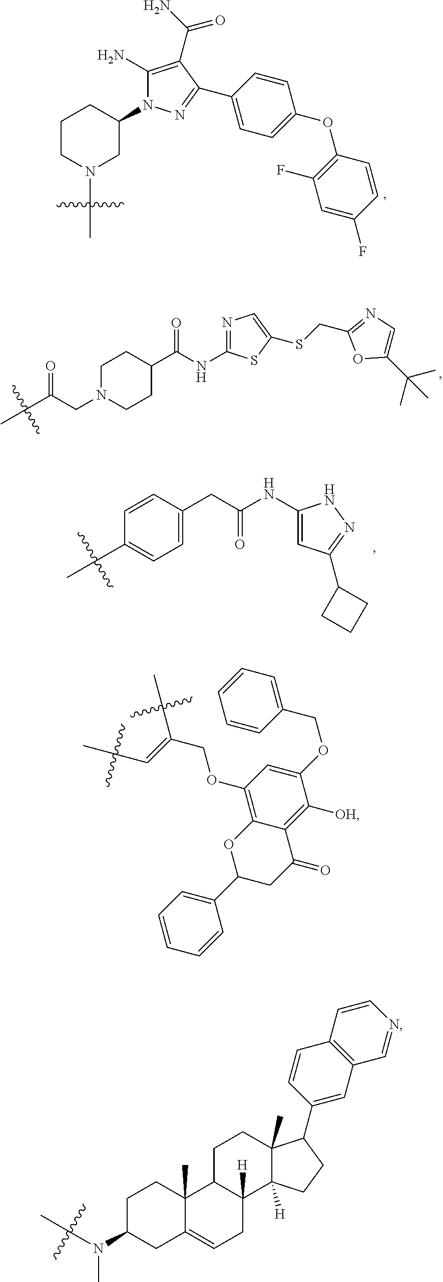

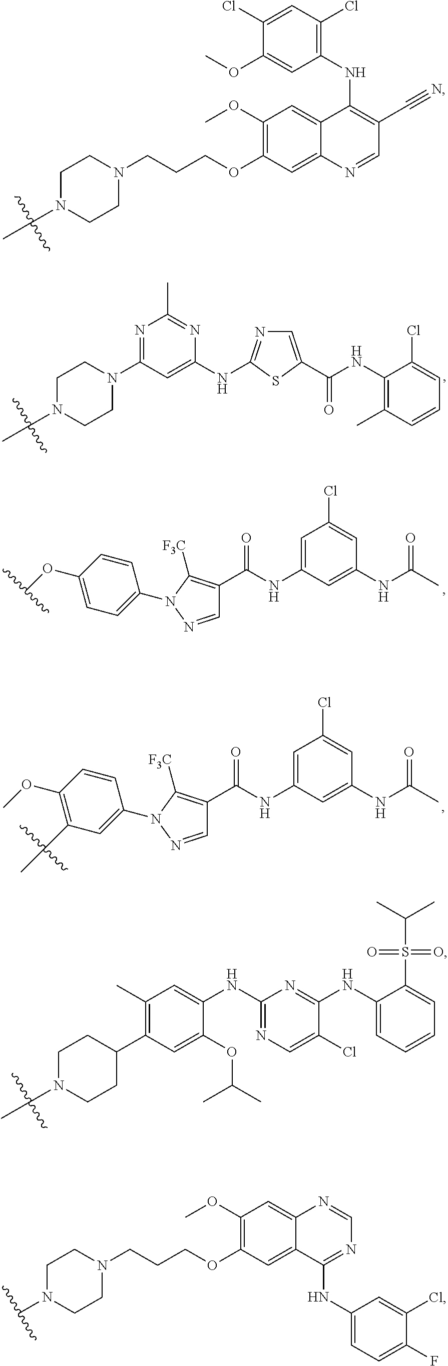

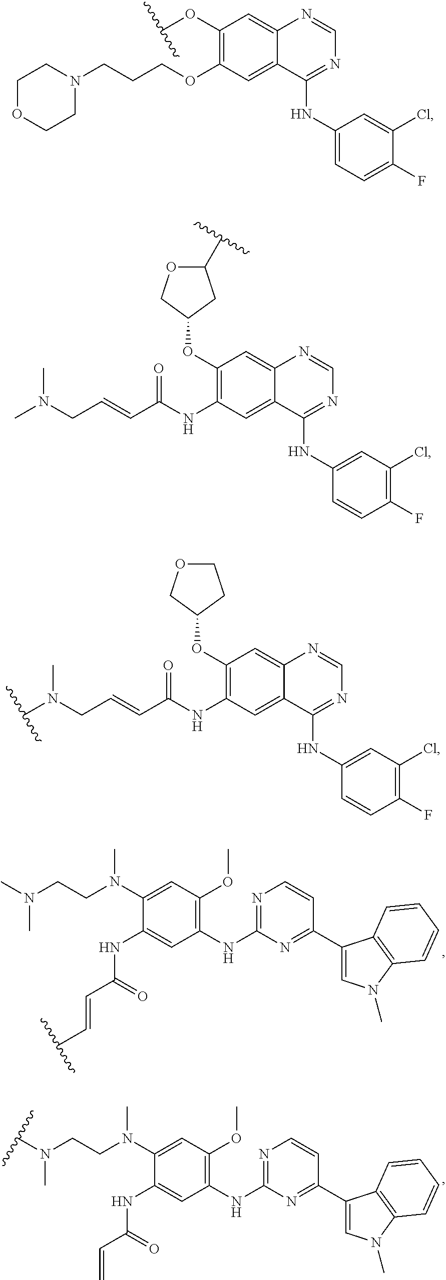 [SMILES: *Oc1cc2ncnc(Nc3ccc(F)c(Cl)c3)c2cc1OCCCN1CCOCC1 ; *C1C[C@H](Oc2cc3ncnc(Nc4ccc(F)c(Cl)c4)c3cc2NC(=O)/C=C/CN(C)C)CO1 ; *N(C)C/C=C/C(=O)Nc1cc2c(Nc3ccc(F)c(Cl)c3)ncnc2cc1O[C@H]1CCOC1 ; */C=C/C(=O)Nc1cc(Nc2nccc(-c3cn(C)c4ccccc34)n2)c(OC)cc1N(C)CCN(C)C ; *N(C)CCN(C)c1cc(OC)c(Nc2nccc(-c3cn(C)c4ccccc34)n2)cc1NC(=O)C=C]

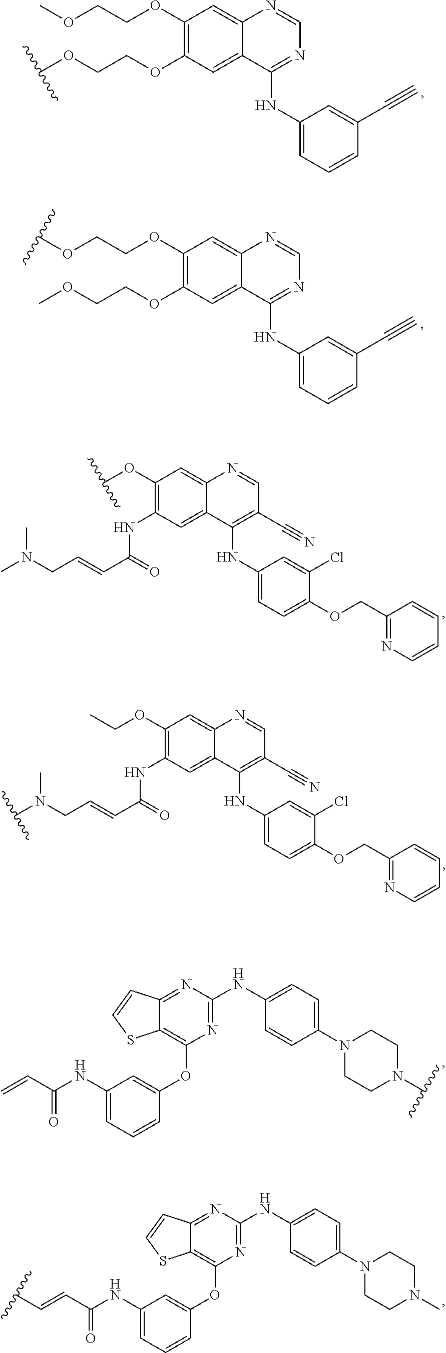

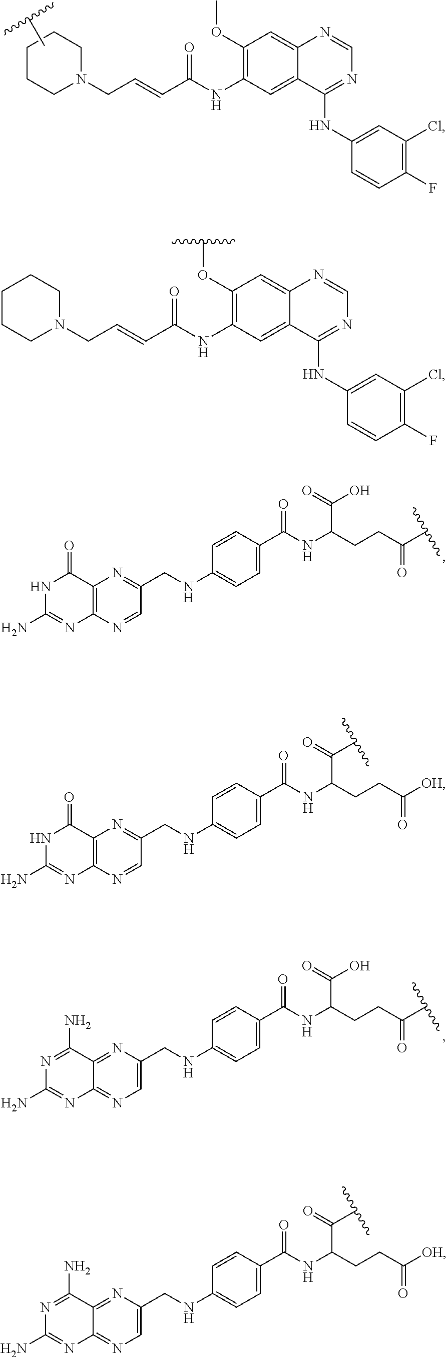

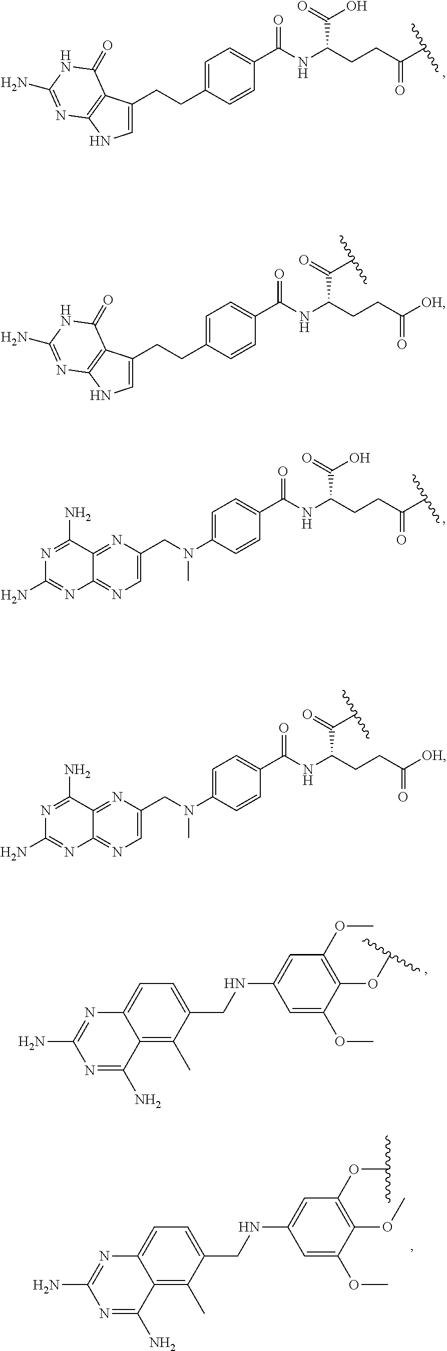 [SMILES: *C(=O)CC[C@H](NC(=O)c1ccc(CCc2c[nH]c3nc(N)[nH]c(=O)c23)cc1)C(=O)O ; *C(=O)[C@H](CCC(=O)O)NC(=O)c1ccc(CCc2c[nH]c3nc(N)[nH]c(=O)c23)cc1 ; *C(=O)CC[C@H](NC(=O)c1ccc(N(C)Cc2cnc3nc(N)nc(N)c3n2)cc1)C(=O)O ; *C(=O)[C@H](CCC(=O)O)NC(=O)c1ccc(N(C)Cc2cnc3nc(N)nc(N)c3n2)cc1 ; *Oc1c(OC)cc(NCc2ccc3nc(N)nc(N)c3c2C)cc1OC ; *Oc1cc(NCc2ccc3nc(N)nc(N)c3c2C)cc(OC)c1OC]

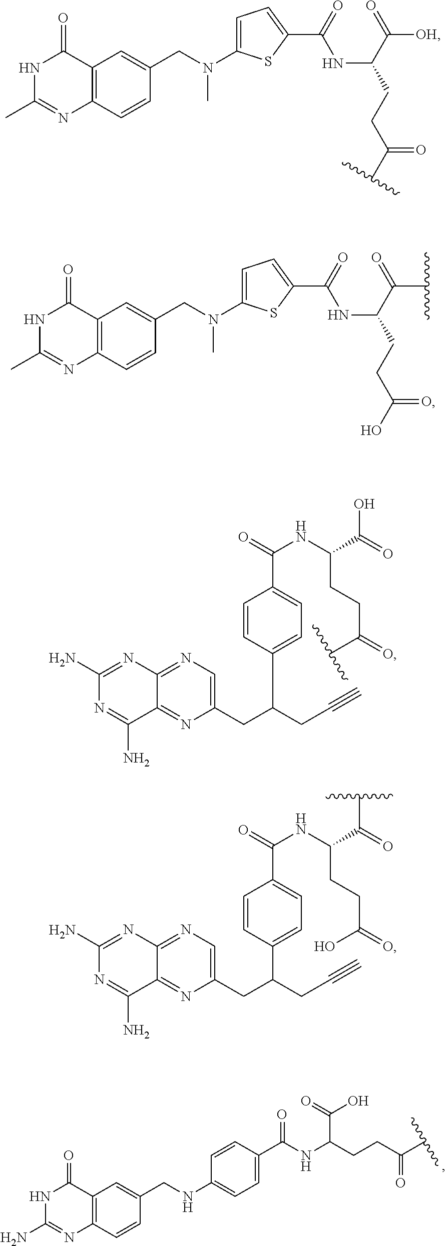 [SMILES: *C(=O)CC[C@H](NC(=O)c1ccc(N(C)Cc2ccc3nc(C)[nH]c(=O)c3c2)s1)C(=O)O ; *C(=O)[C@H](CCC(=O)O)NC(=O)c1ccc(N(C)Cc2ccc3nc(C)[nH]c(=O)c3c2)s1 ; *C(=O)CC[C@H](NC(=O)c1ccc(C(CC#C)Cc2cnc3nc(N)nc(N)c3n2)cc1)C(=O)O ; *C(=O)[C@H](CCC(=O)O)NC(=O)c1ccc(C(CC#C)Cc2cnc3nc(N)nc(N)c3n2)cc1 ; *C(=O)CCC(NC(=O)c1ccc(NCc2ccc3nc(N)[nH]c(=O)c3c2)cc1)C(=O)O]

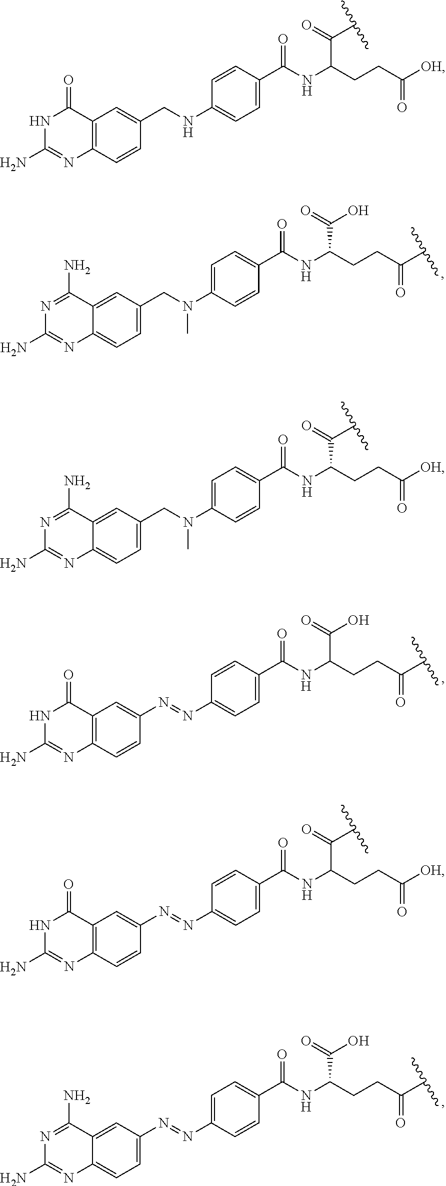 [SMILES: *C(=O)C(CCC(=O)O)NC(=O)c1ccc(NCc2ccc3nc(N)[nH]c(=O)c3c2)cc1 ; *C(=O)CC[C@H](NC(=O)c1ccc(N(C)Cc2ccc3nc(N)nc(N)c3c2)cc1)C(=O)O ; *C(=O)[C@H](CCC(=O)O)NC(=O)c1ccc(N(C)Cc2ccc3nc(N)nc(N)c3c2)cc1 ; *C(=O)CCC(NC(=O)c1ccc(/N=N/c2ccc3nc(N)[nH]c(=O)c3c2)cc1)C(=O)O ; *C(=O)C(CCC(=O)O)NC(=O)c1ccc(/N=N/c2ccc3nc(N)[nH]c(=O)c3c2)cc1 ; *C(=O)CC[C@H](NC(=O)c1ccc(/N=N/c2ccc3nc(N)nc(N)c3c2)cc1)C(=O)O]

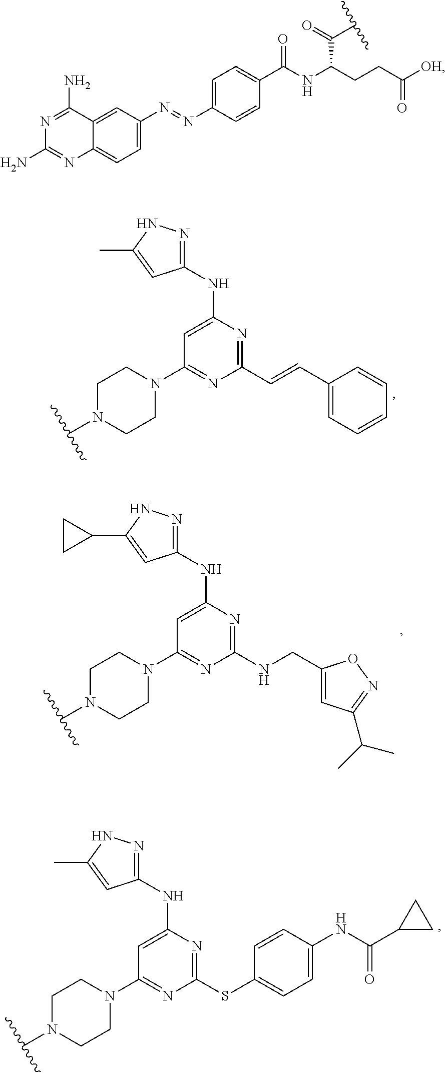 [SMILES: *C(=O)[C@H](CCC(=O)O)NC(=O)c1ccc(/N=N/c2ccc3nc(N)nc(N)c3c2)cc1 ; *N1CCN(c2cc(Nc3cc(C)[nH]n3)nc(/C=C/c3ccccc3)n2)CC1 ; *N1CCN(c2cc(Nc3cc(C4CC4)[nH]n3)nc(NCc3cc(C(C)C)no3)n2)CC1 ; *N1CCN(c2cc(Nc3cc(C)[nH]n3)nc(Sc3ccc(NC(=O)C4CC4)cc3)n2)CC1]

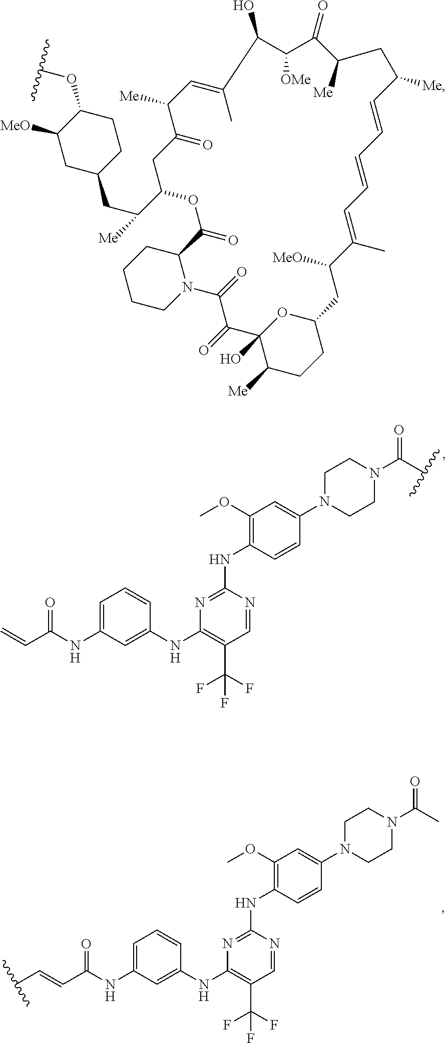 [SMILES: *O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; *C(=O)N1CCN(c2ccc(Nc3ncc(C(F)(F)F)c(Nc4cccc(NC(=O)C=C)c4)n3)c(OC)c2)CC1 ; */C=C/C(=O)Nc1cccc(Nc2nc(Nc3ccc(N4CCN(C(C)=O)CC4)cc3OC)ncc2C(F)(F)F)c1]

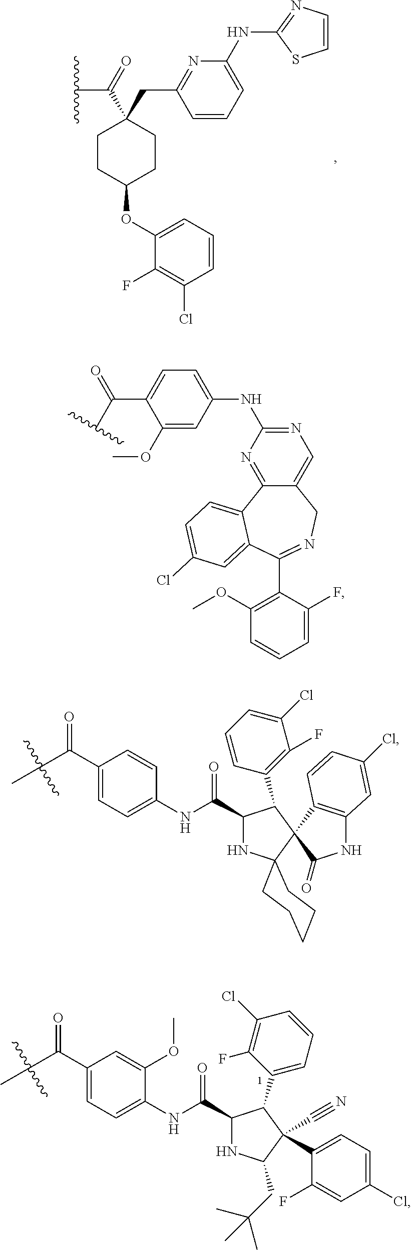

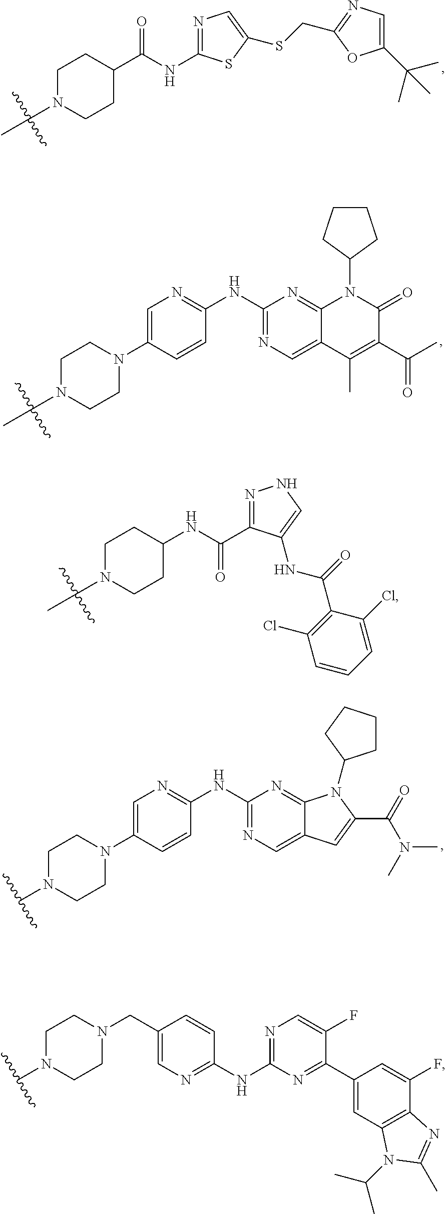

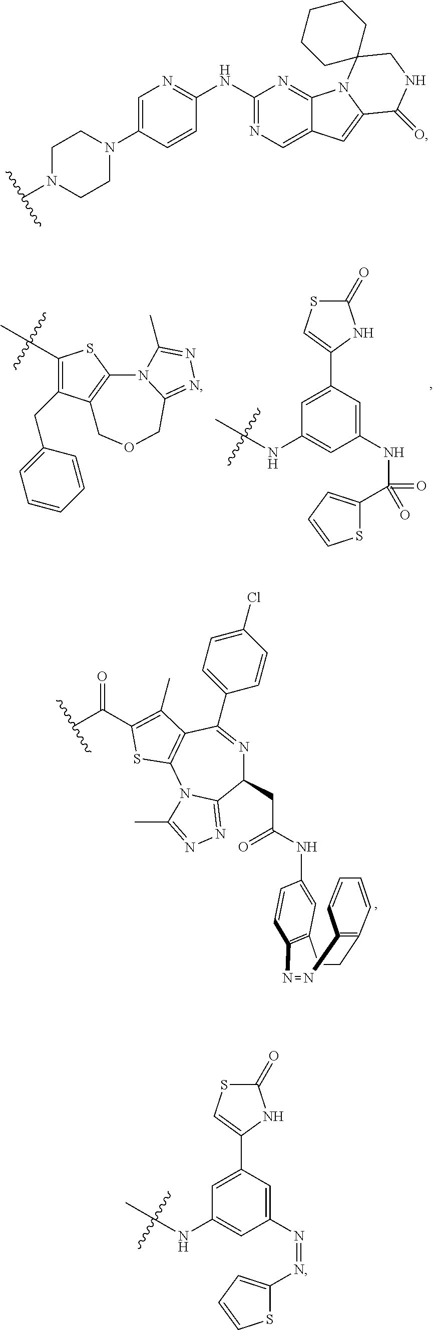

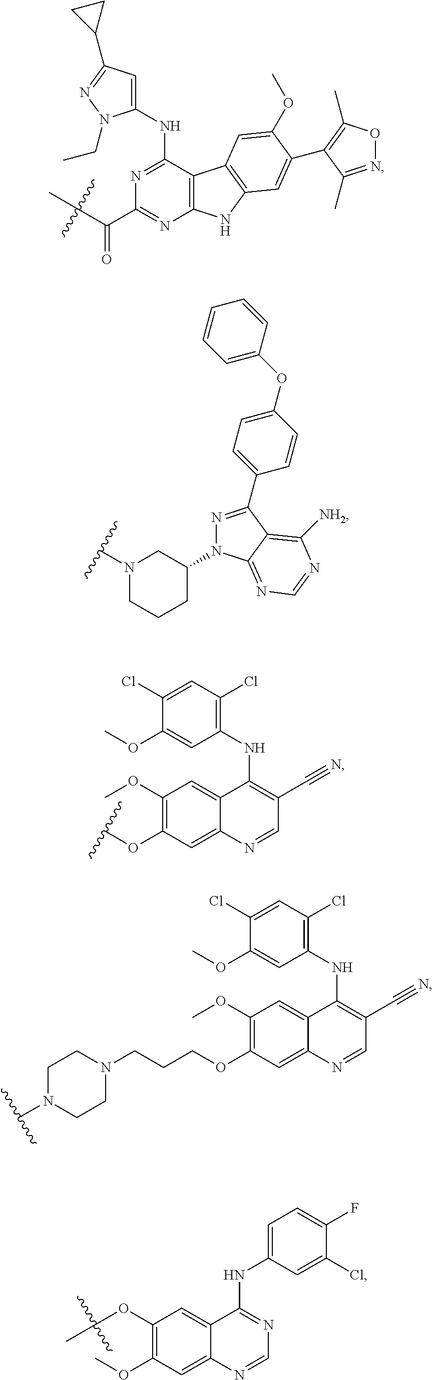 [SMILES: *C(=O)c1nc(Nc2cc(C3CC3)nn2CC)c2c(n1)[nH]c1cc(-c3c(C)noc3C)c(OC)cc12 ; *N1CCC[C@@H](n2nc(-c3ccc(Oc4ccccc4)cc3)c3c(N)ncnc32)C1 ; *Oc1cc2ncc(C#N)c(Nc3cc(OC)c(Cl)cc3Cl)c2cc1OC ; *N1CCN(CCCOc2cc3ncc(C#N)c(Nc4cc(OC)c(Cl)cc4Cl)c3cc2OC)CC1 ; *Oc1cc2c(Nc3ccc(F)c(Cl)c3)ncnc2cc1OC]

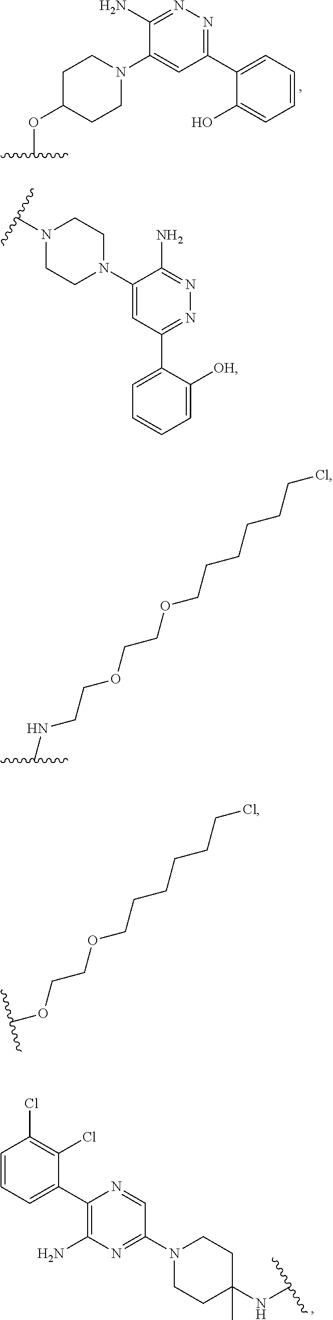 [SMILES: *OC1CCN(c2cc(-c3ccccc3O)nnc2N)CC1 ; *N1CCN(c2cc(-c3ccccc3O)nnc2N)CC1 ; *NCCOCCOCCCCCCCl ; *OCCOCCCCCCCl ; *NC1(C)CCN(c2cnc(-c3cccc(Cl)c3Cl)c(N)n2)CC1]

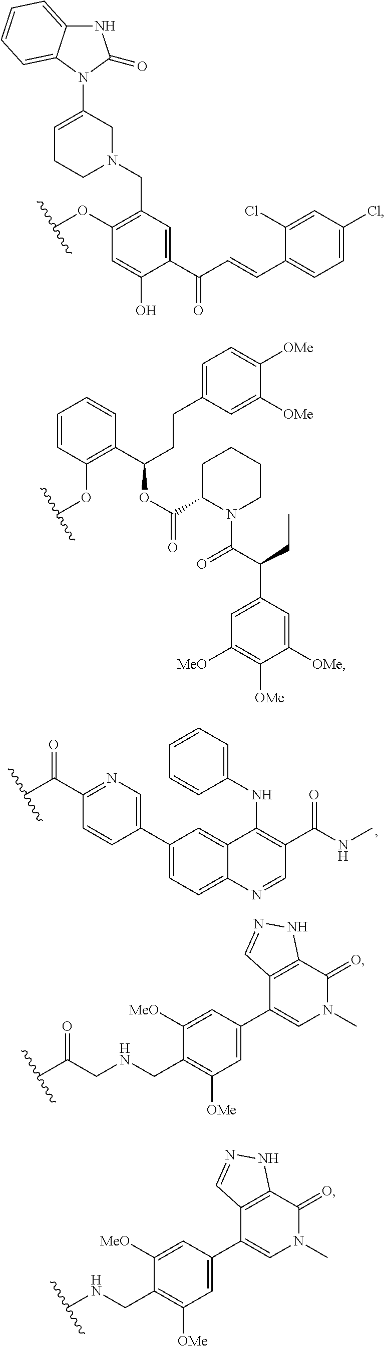

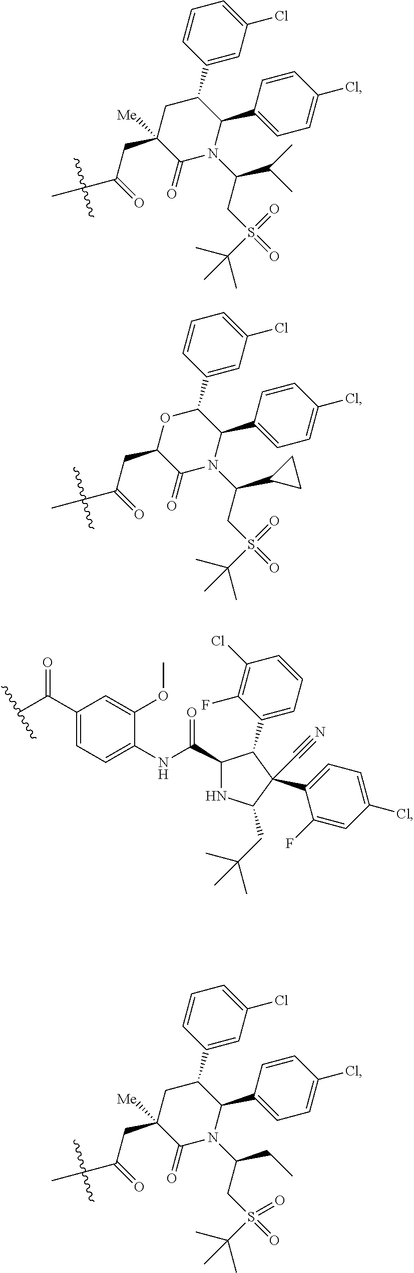

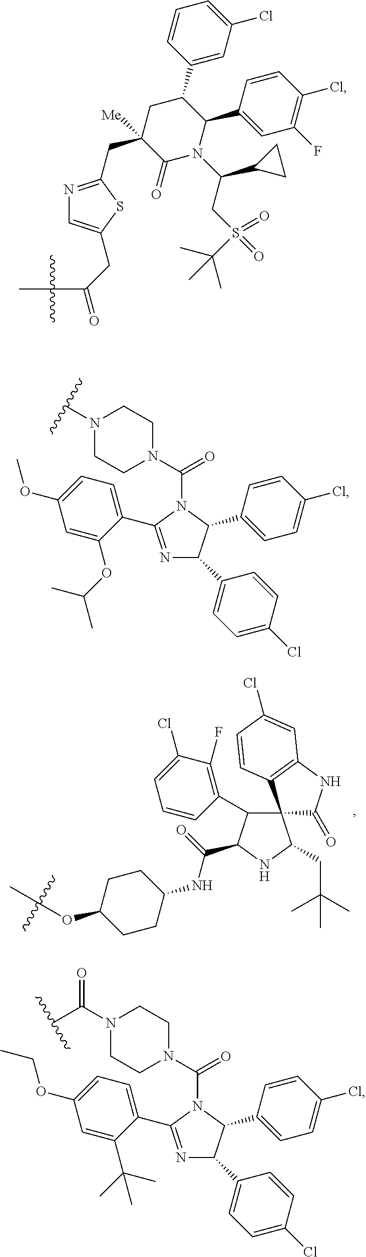

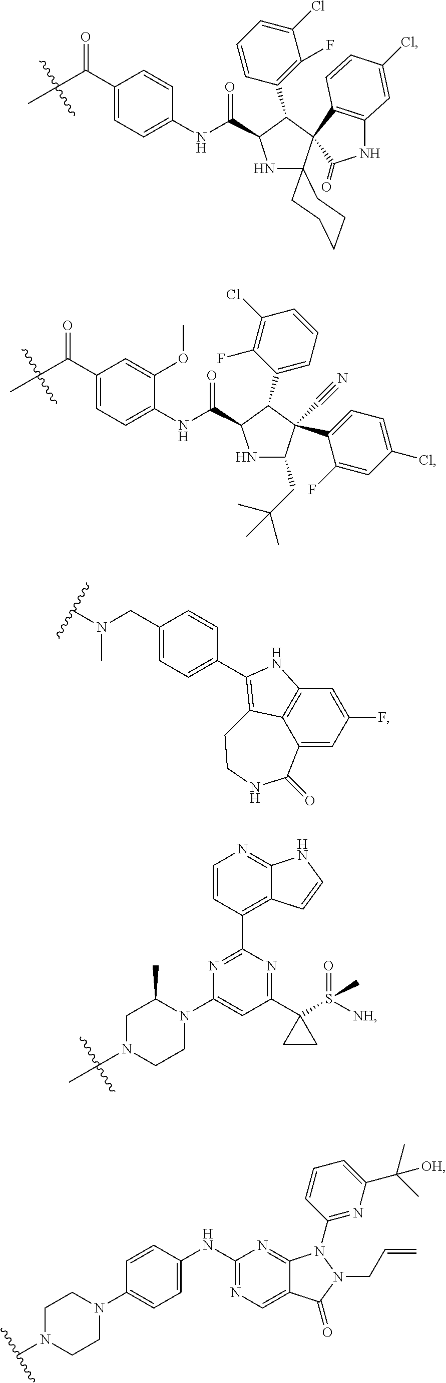

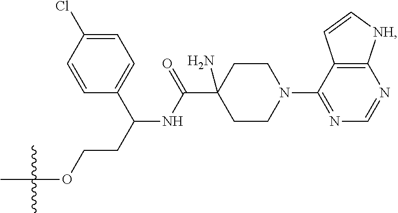 [SMILES: *OCCC(NC(=O)C1(N)CCN(c2ncnc3[nH]ccc23)CC1)c1ccc(Cl)cc1]

GQEDATADDQYQQY (SEQ ID NO:4), and the like, and combinations thereof.

Additional non-limiting examples of ligands for target proteins include ligands formed from the following: an Hsp90 inhibitor, a kinase inhibitor, a phosphatase inhibitor, an HDM2/MDM2 inhibitor, a compound which targets human BET Bromodomain-containing proteins, an HDAC inhibitor, a human lysine methyltransferase inhibitor, a compound targeting RAF receptor, a compound targeting FKBP, an angiogenesis inhibitor, an immunosuppressive compound, a compound targeting an arylhydrocarbon receptor, a compound targeting an androgen receptor, a compound targeting an estrogen receptor, a compound targeting an estrogen related receptor, a compound targeting a thyroid hormone receptor, a compound targeting HIV protease, a compound targeting HIV integrase, a compound targeting HCV protease, a compound targeting acyl protein thioesterase 1 and/or 2, TANK-binding kinase 1 (TBK1), estrogen receptor C. (ERO), bromodomain-containing protein 4 (BRD4), a compound targeting ERK1/2/3, a compound targeting Weel, a compound targeting ATR, a compound targeting PARP1, a compound targeting PARP2, a compound targeting IDH1, a compound targeting IDH2, a compound targeting human methionine adenosyltransferase 2A, a compound targeting TRKA, a compound targeting TRKB, a compound targeting TRKC, a compound targeting mutant p53, a compound targeting NLRP3, a compound targeting cGAS, a compound targeting STING, a compound targeting MAVS, androgen receptor (AR), and c-Myc. 14. Additional examples of ligands include ligands formed from the following: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-73.9358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFRTK inhibitor, an IGFR-TK inhibitor, an anti-HGF antibody, a PI3 kinase inhibitors, an AKT inhibitor, an mTORC1/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, aZd2171, batabulin, ofatumumab, Zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR KRX-0402, lucanthone, LY317615, neuradiab, vitespan, Rita 744, Sdx 102, talampanel, atrasentan, Xr311, romidepsin, ADS-100380, Sunitinib, 5-fluorouracil, Vorinostat, etoposide, gemcitabine, doxorubicin, liposomal doxorubicin, 5′-deoxy-5-fluorouridine, Vincristine, temozolomide, ZK-304709, seliciclib; PD0325901, AZD-6244, capecitabine, L-Glutamic acid, N-4-2-(2-amino-4,7-dihydro-4-oxo-1H-pyrrolo2,3-dipyrimidin-5-yl)ethylben Zoyl-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11.CHIR-258.); 3-5-(methyl sulfonylpiperadinemethyl)-indolyl-quinolone, vatalanib, AG-013736, AVE-0005, the acetate salt of D-Ser(But) 6, Azgly 10 (pyro-Glu-His-Trp Ser-Tyr-D-Ser(But)-Leu-Arg-Pro-Azgly-NH acetate CsoHsNOia-(CHO), where X=1 to 2.4, goserelin acetate, leuprolide acetate, triptorelin pamoate, medroXyprogesterone acetate, hydroxyprogesterone caproate, megestrol acetate, raloxifene, bicalutamide, flutamide, nilutamide, megestrol acetate, CP-7247 14: TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoylanalide hydroxamic acid, valproic acid, trichostatin A, FK-228, SU 11248, Sorafenib, KRN951, aminoglutethimide, amsacrine, anagrelide, L-asparaginase, Bacillus Calmette-Guerin (BCG) vaccine, adriamycin, bleomycin, buserelin, buSulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, Oxaliplatin, pamidronate, pentostatin, plicamycin, porfimer, procarbaZine, raltitrexed, rituximab, Streptozocin, teniposide, testosterone, thalidomide, thioguanine, thiotepa, tretinoin, Vindesine, 13-cis-retinoic acid, phenylalanine mustard, uracil mustard, estramustine, altretamine, floxuridine, 5-deoxyurdine, cytosine arabinoside, 6-mecaptopurine, deoxycoformycin, calcitriol, valrubicin, mithramycin, vinblastine, Vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxy fene, Spironolactone, finasteride, cimitidine, trastuzumab, denileukin diftitox, gefitinib, bortezimib, paclitaxel, cremophor-free paclitaxel, docetaxel, epothilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD184352, rapamycin, 40-O-(2-hydroxyethyl)-rapamycin, temsirolimus, AP-23573, RAD001, ABT-578, BC-210, LY294.002, LY292223, LY292696, LY293.684, LY293646, wortmannin, ZM336372, L-779,450, PEG-filgrastim, darbepoetin, erythropoietin, granulocyte colony-stimulating factor, Zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, andro gens, decitabine, hexamethylmelamine, bexarotene, to situmomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and the like, and combinations thereof.

In various examples, a PHOTAC of the present disclosure has the following structure:

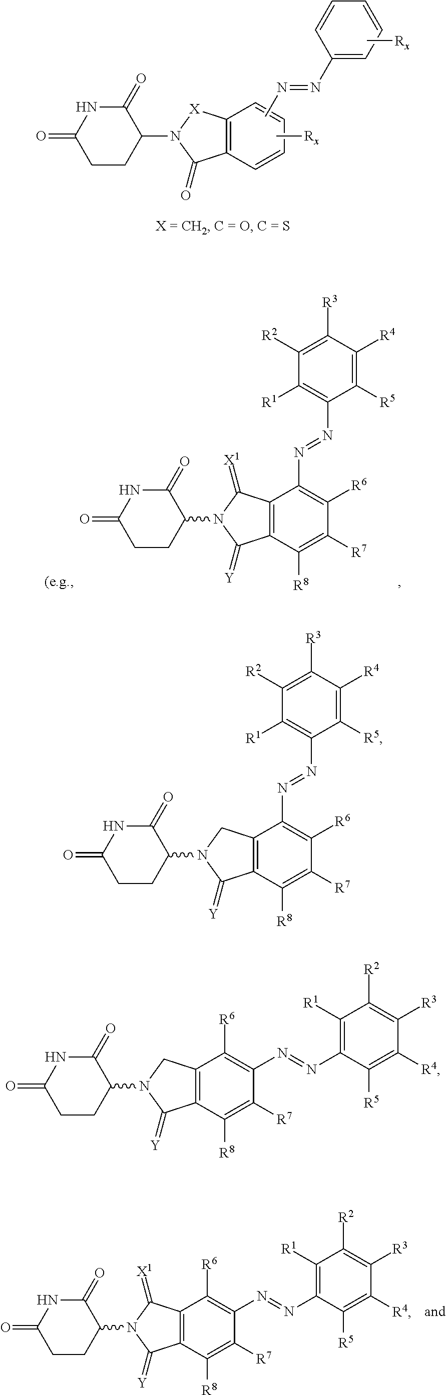

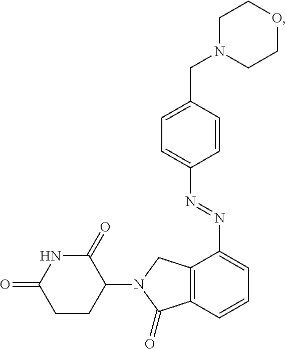 [SMILES: O=C1CCC(N2Cc3c(/N=N/c4ccc(CN5CCOCC5)cc4)cccc3C2=O)C(=O)N1]

and the like), where Y and X¹ are independently chosen from O and S; R¹-R⁷ are independently chosen from F, Cl, Me, or OMe; R is H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂, and where R′—R⁵ are also chosen from L-B*, where L is optional and is a linker and B* is a ligand for a target protein, and x is 0, 1, 2, 3, 4, or 5.

In various examples, a PHOTAC of the present disclosure has the following structure:

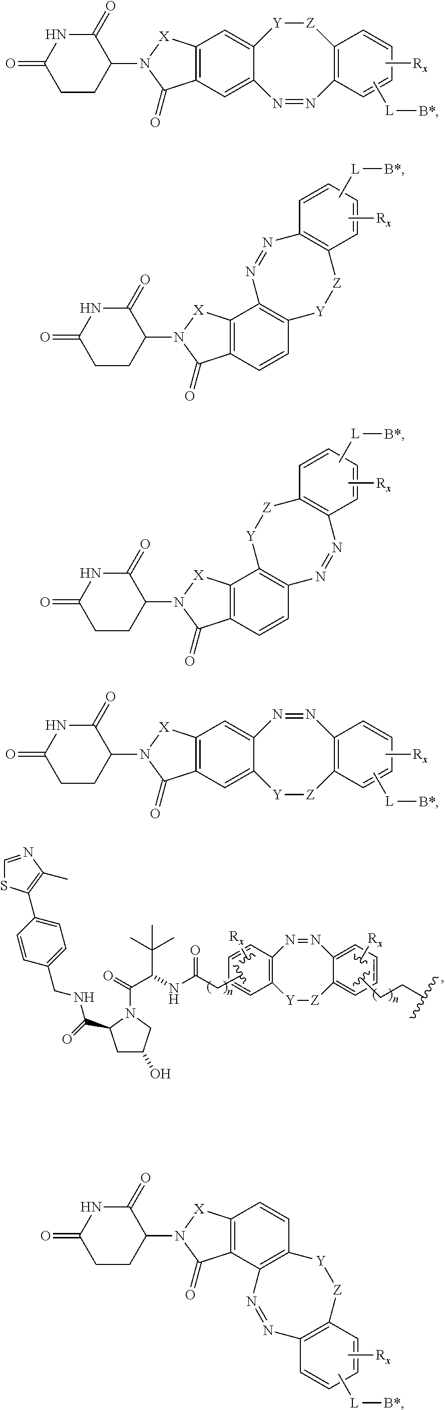

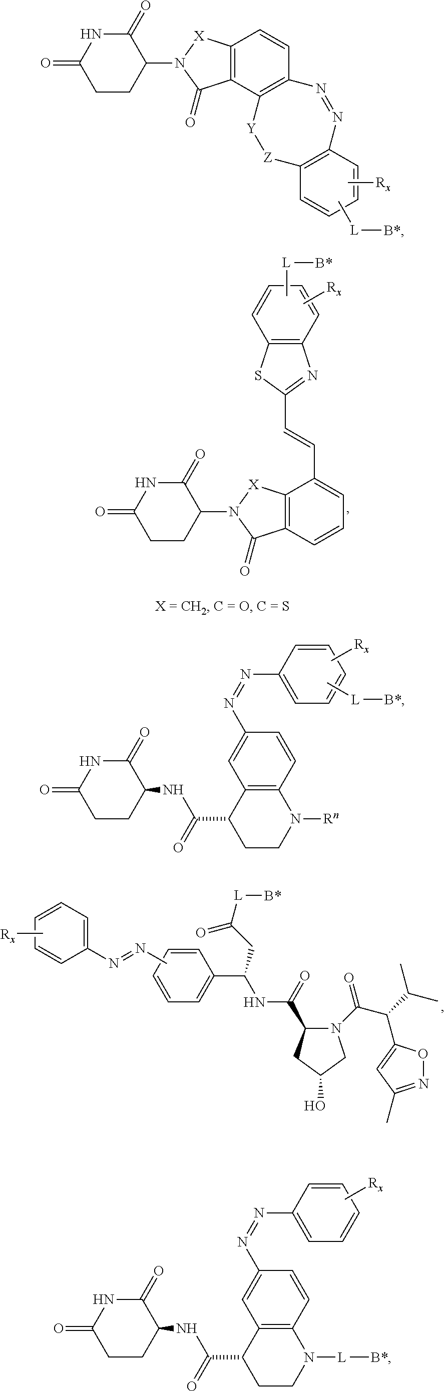

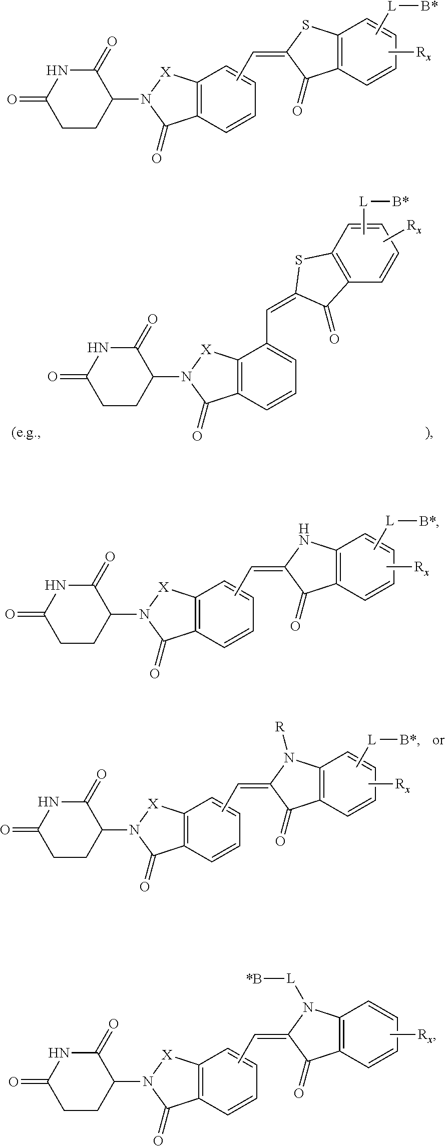

where X is methylene, C═O, or C═S; Y and Z are independently methylene, ethylene, O, —NH—, or S; R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂, and L-B*, where L is optional and is a linker and B* is a ligand for a target protein, and x is 0, 1, 2, 3, 4, or 5.

In various other examples, a PHOTAC of the present disclosure has the following structure:

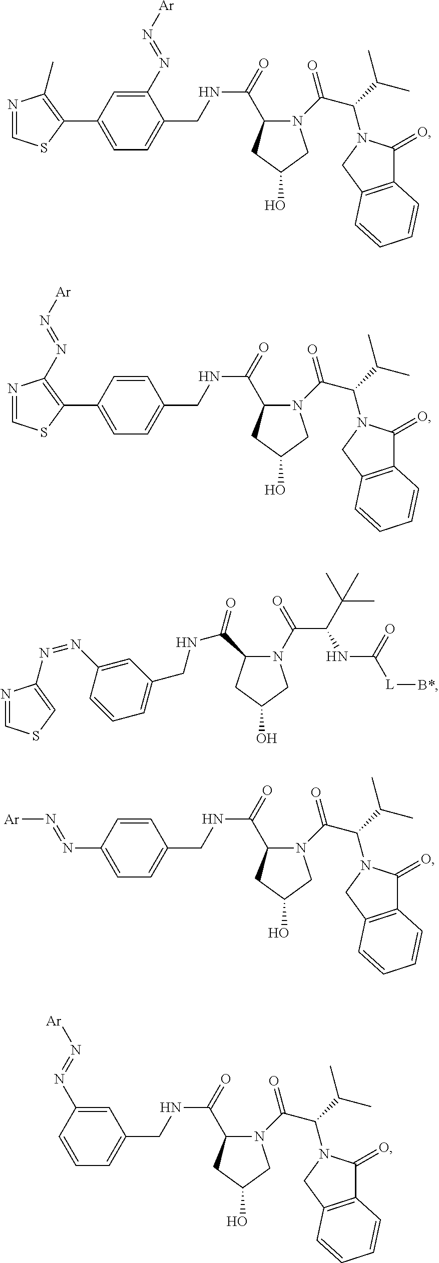

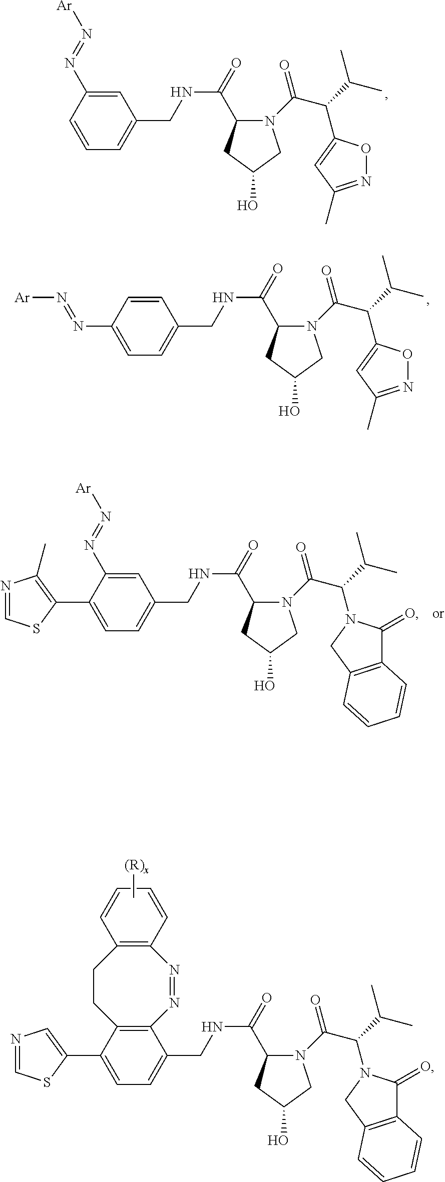

where x is, in various examples, 0, 1, 2, 3, or 4 and R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, and L-B*, where L is optional and is a linker and B* is a ligand for a target protein; Ar is an aryl group

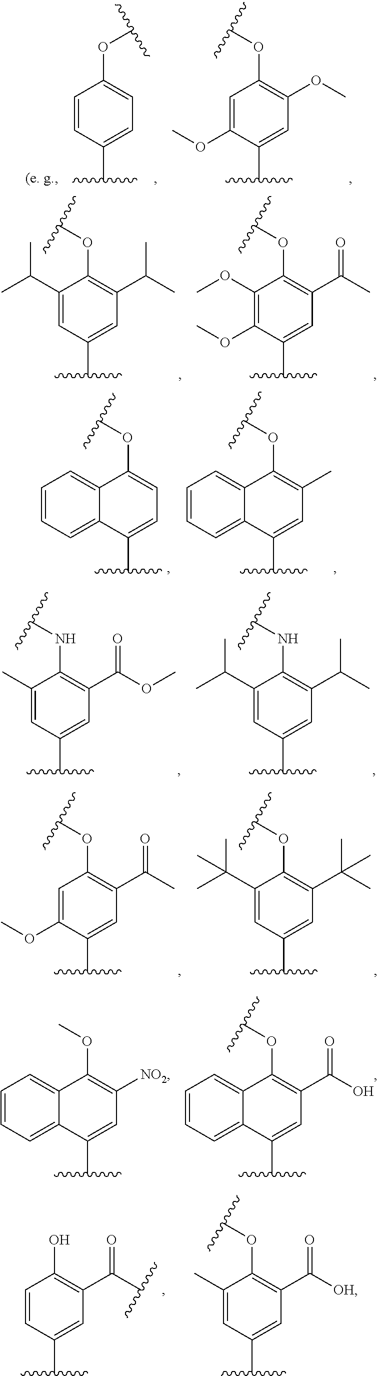

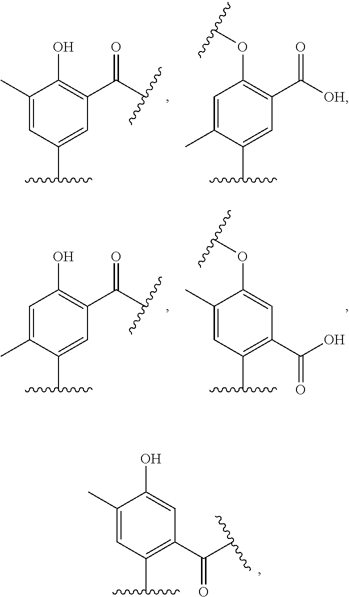

and the like) that is further attached to a linker that is attached to a ligand for a target protein (e.g., protein of interest) or to a ligand of interest.

In various examples, a PHOTAC of the present disclosure has the following structure:

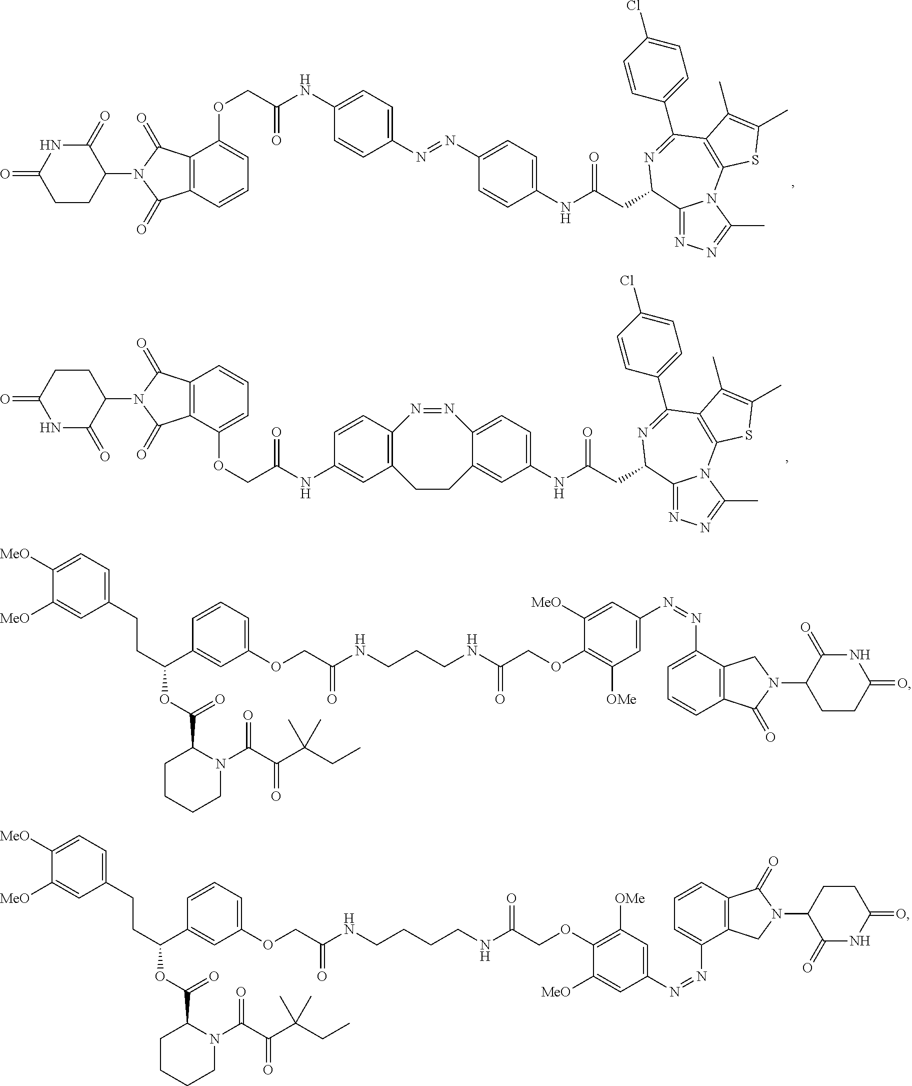

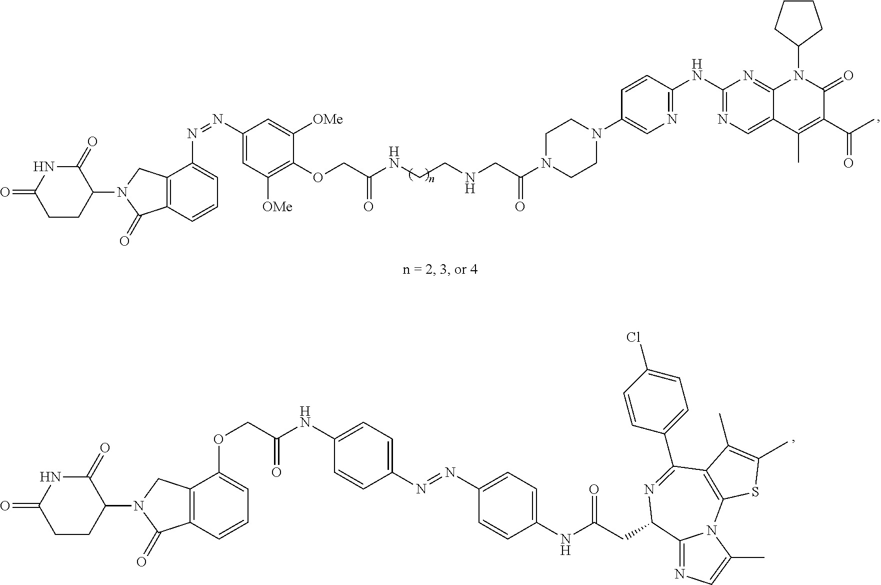

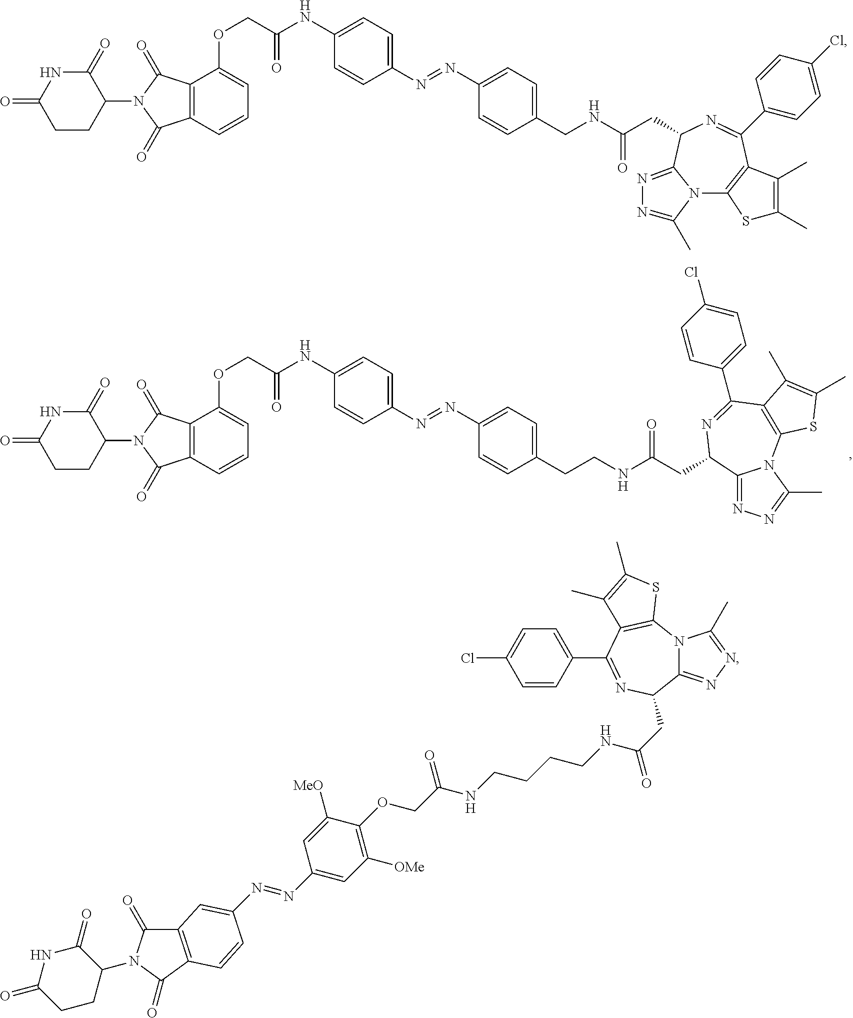

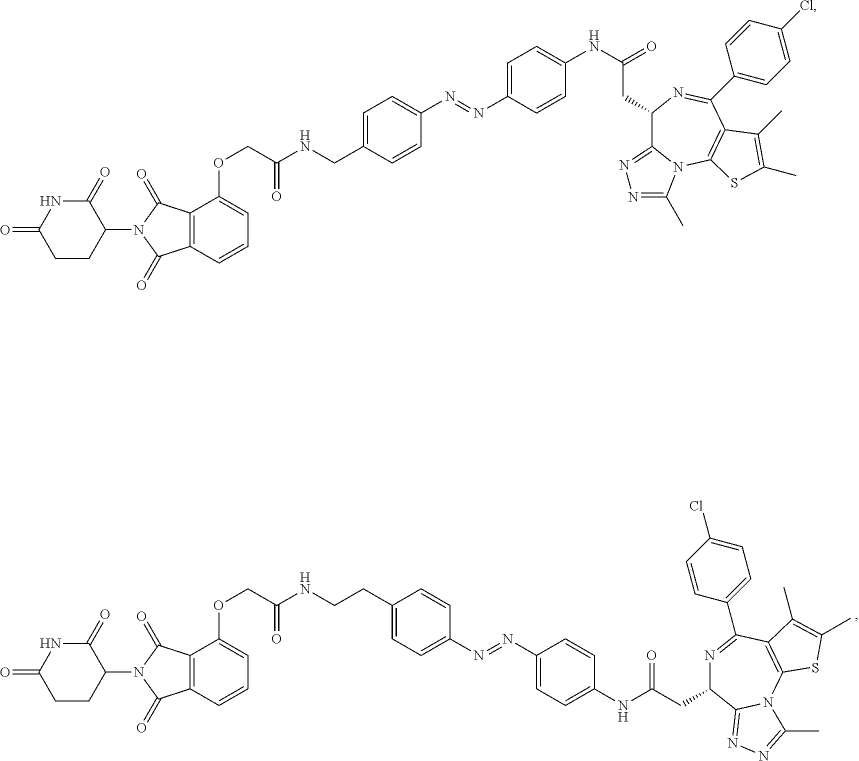 [SMILES: Cc1sc2c(c1C)C(c1ccc(Cl)cc1)=N[C@@H](CC(=O)Nc1ccc(/N=N/c3ccc(CNC(=O)COc4cccc5c4C(=O)N(C4CCC(=O)NC4=O)C5=O)cc3)cc1)c1nnc(C)n1-2 ; Cc1sc2c(c1C)C(c1ccc(Cl)cc1)=N[C@@H](CC(=O)Nc1ccc(/N=N/c3ccc(CCNC(=O)COc4cccc5c4C(=O)N(C4CCC(=O)NC4=O)C5=O)cc3)cc1)c1nnc(C)n1-2]

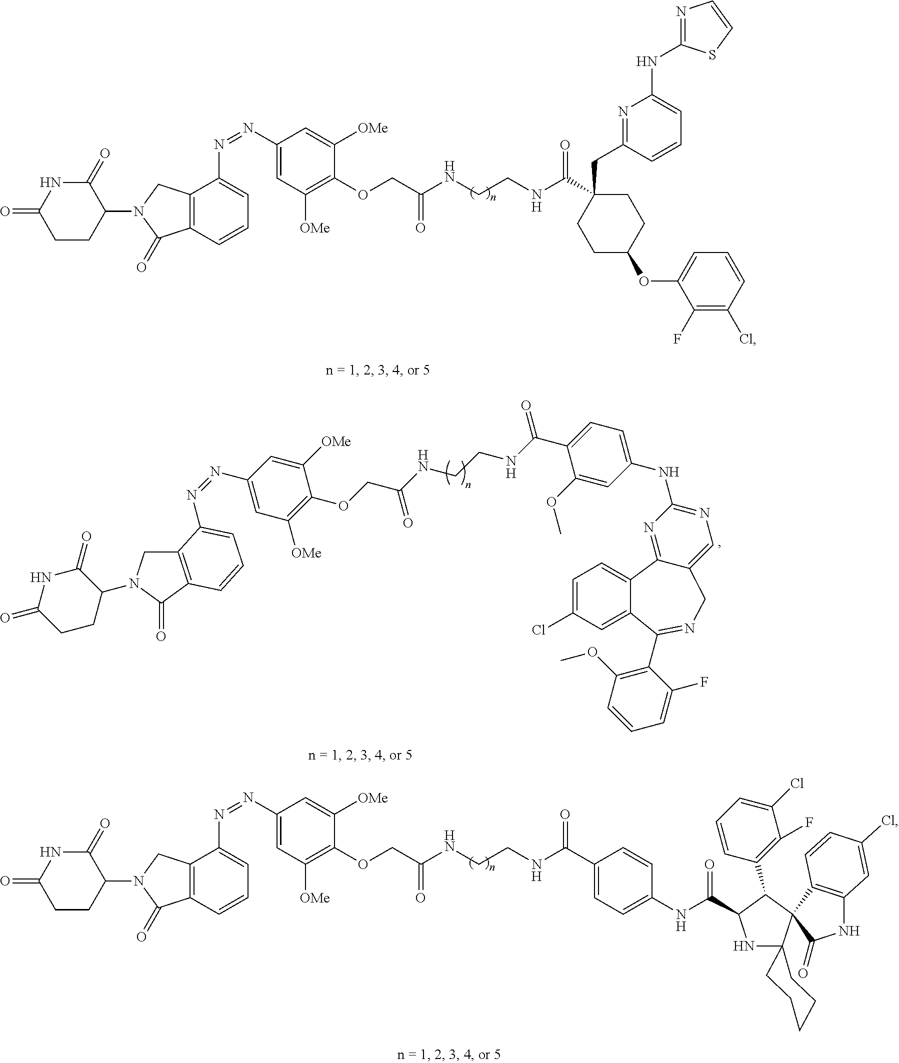

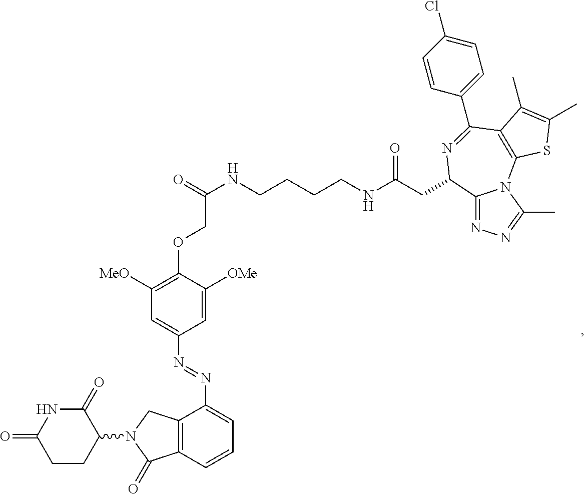

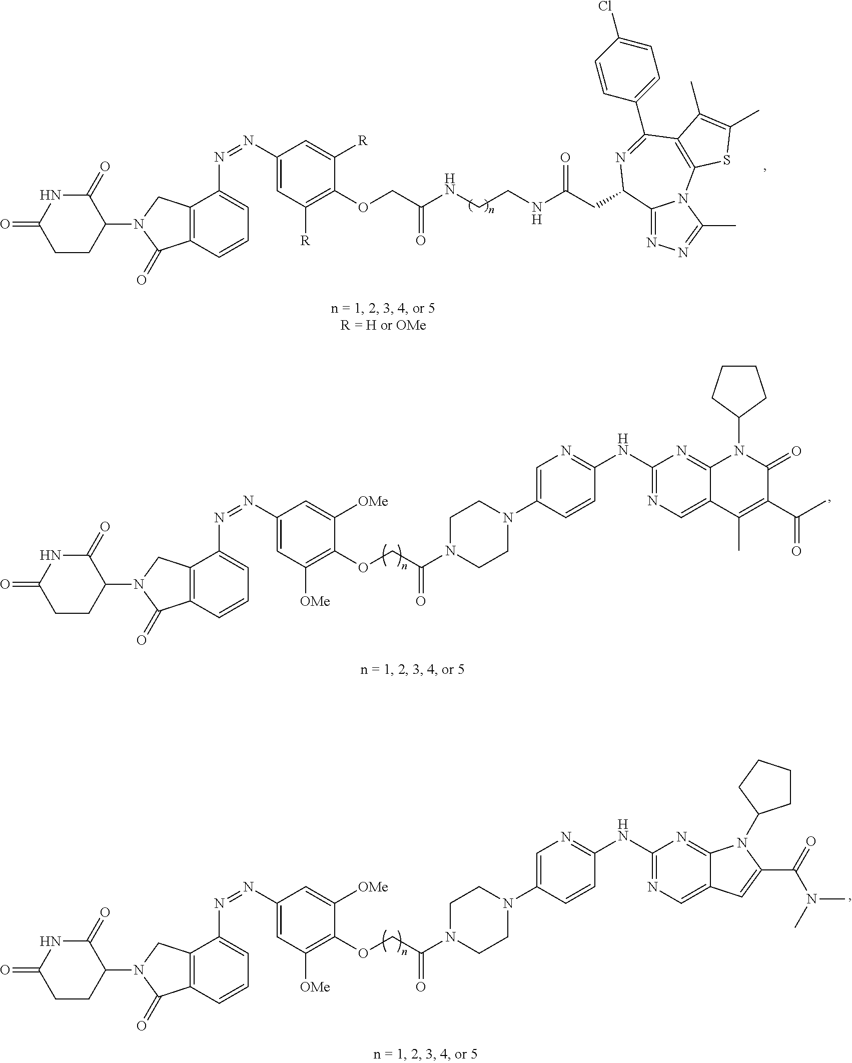

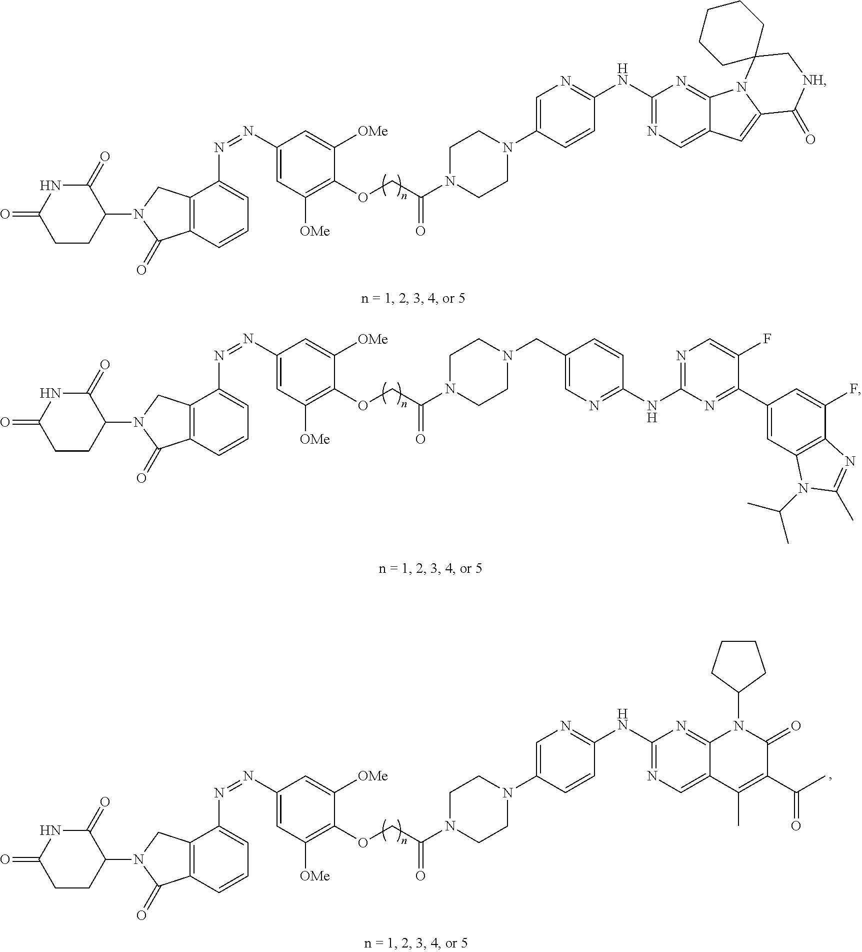

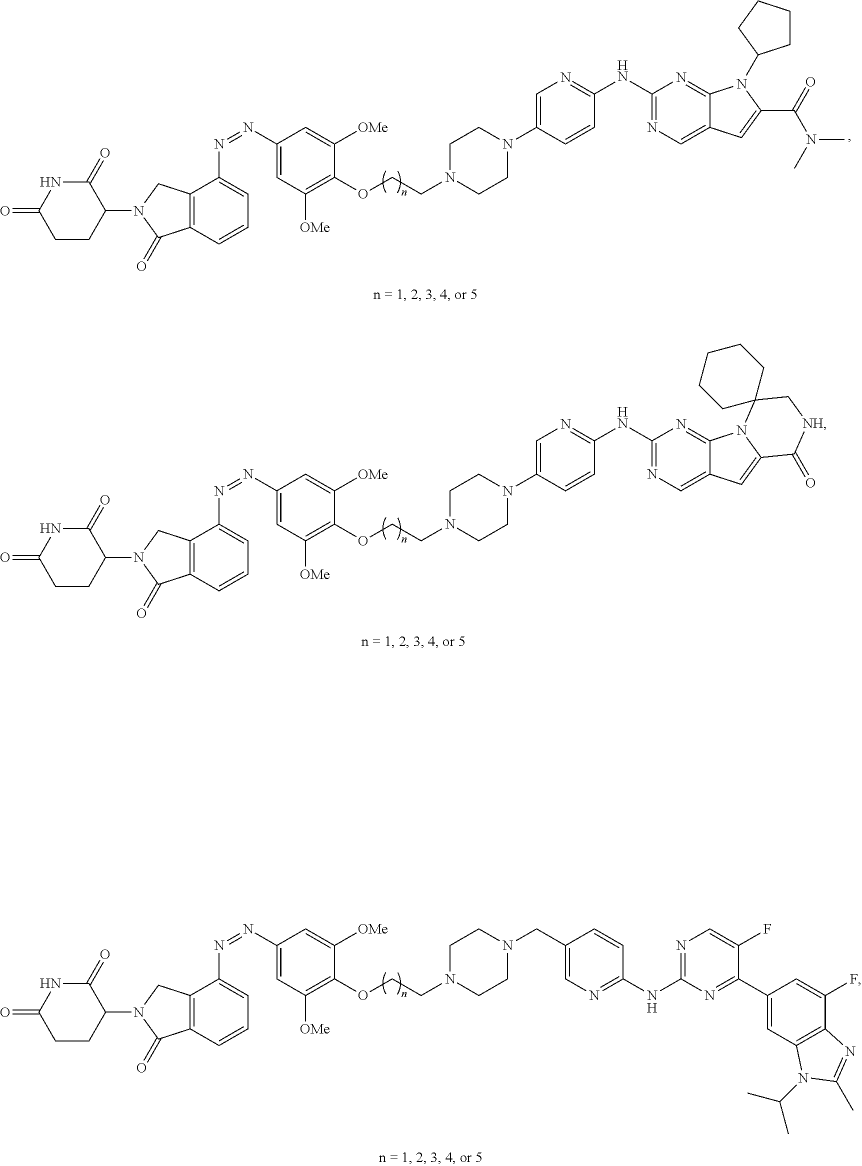

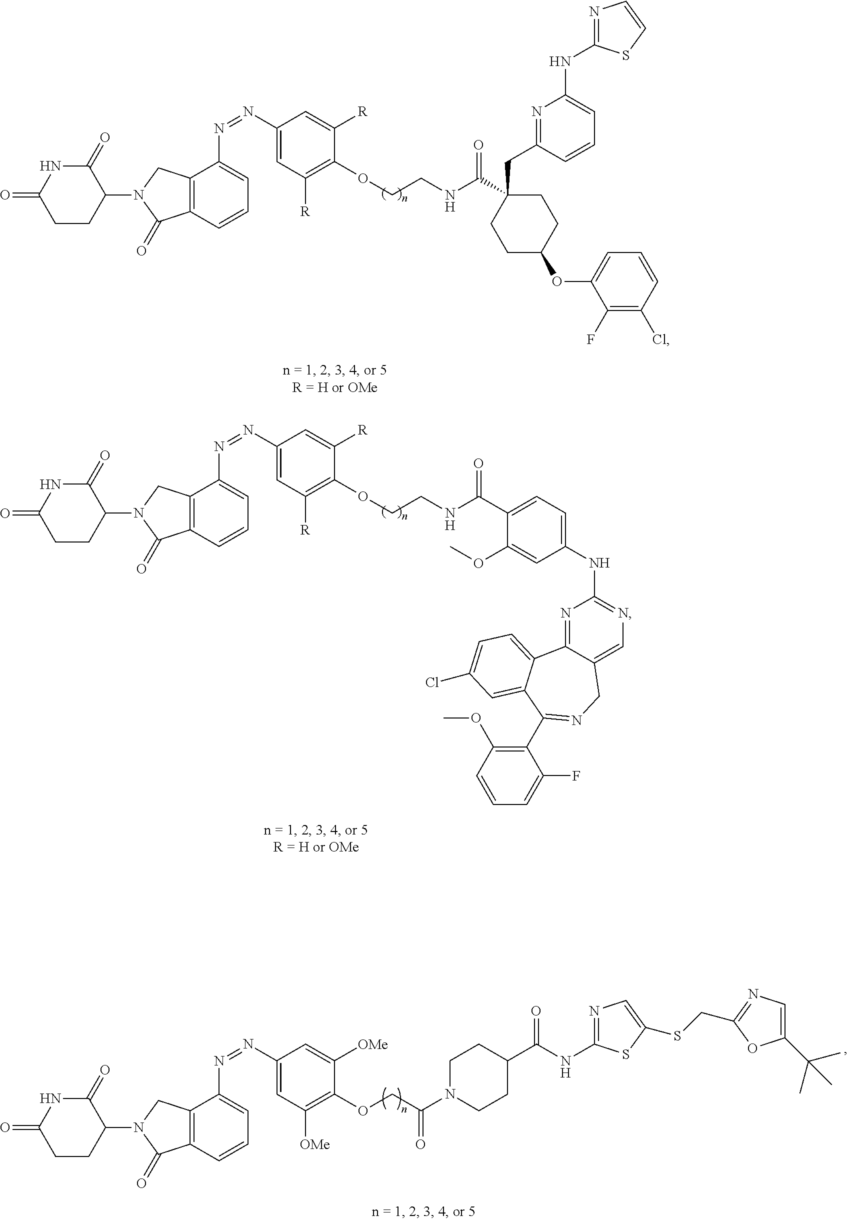

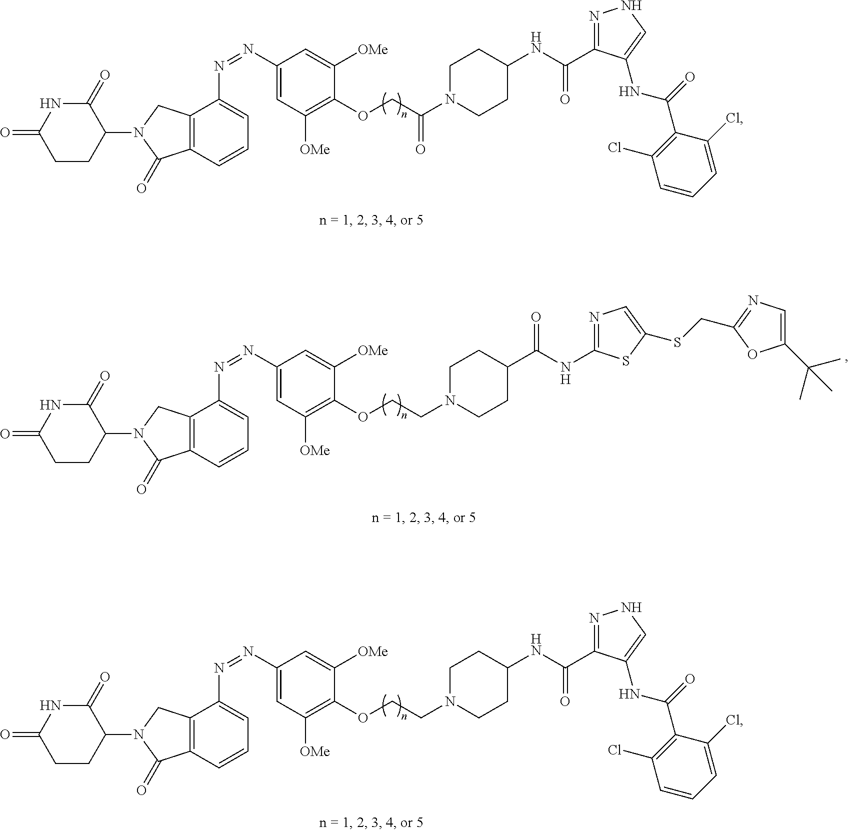

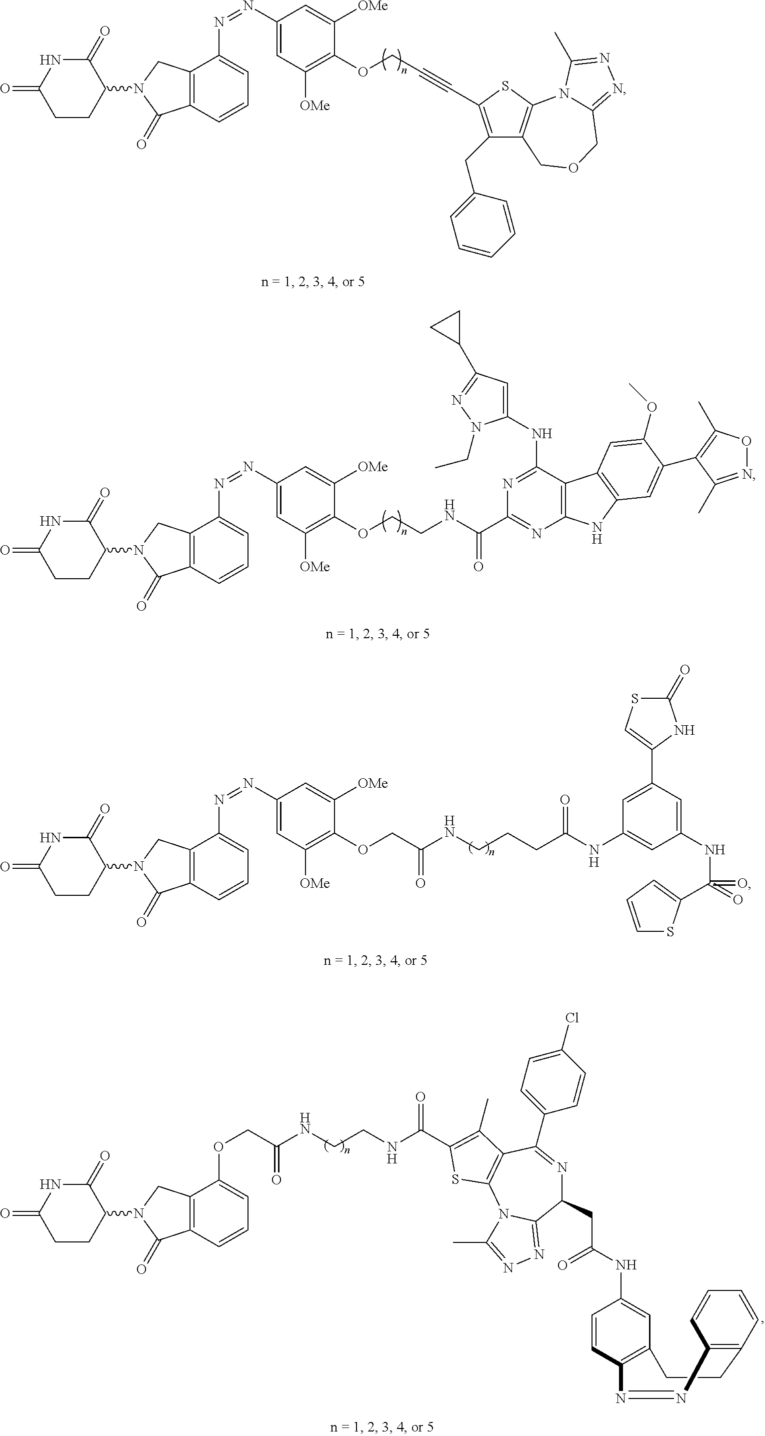

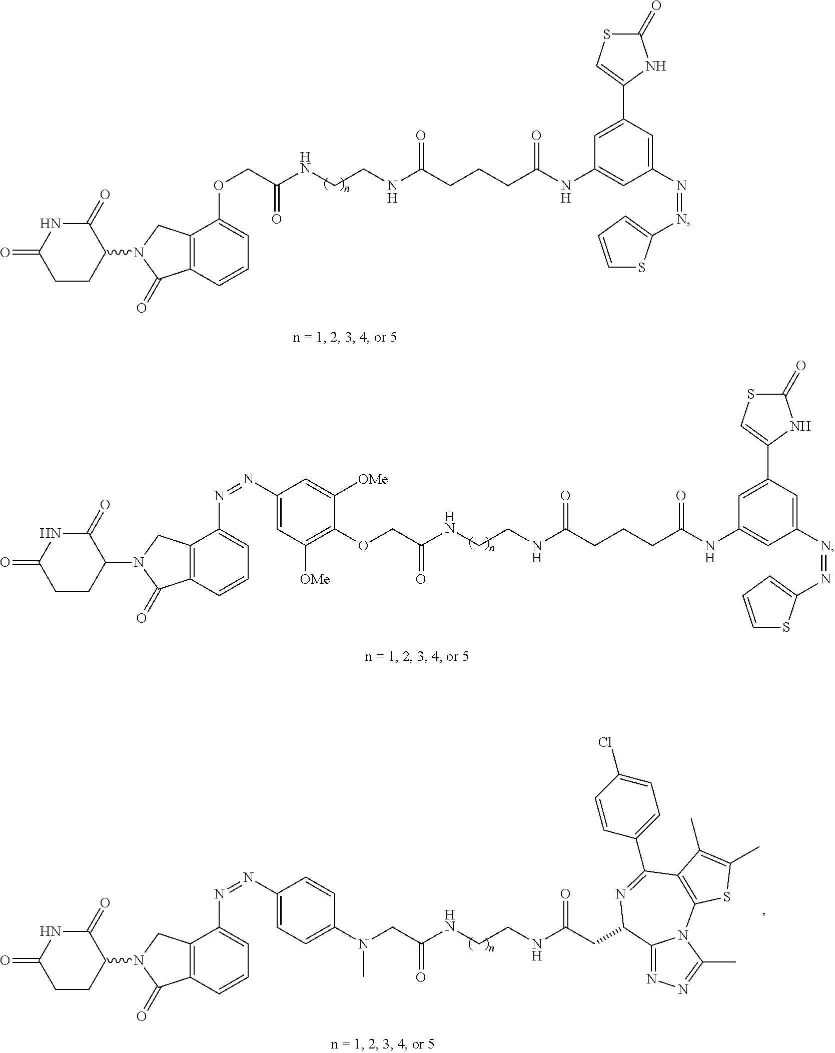

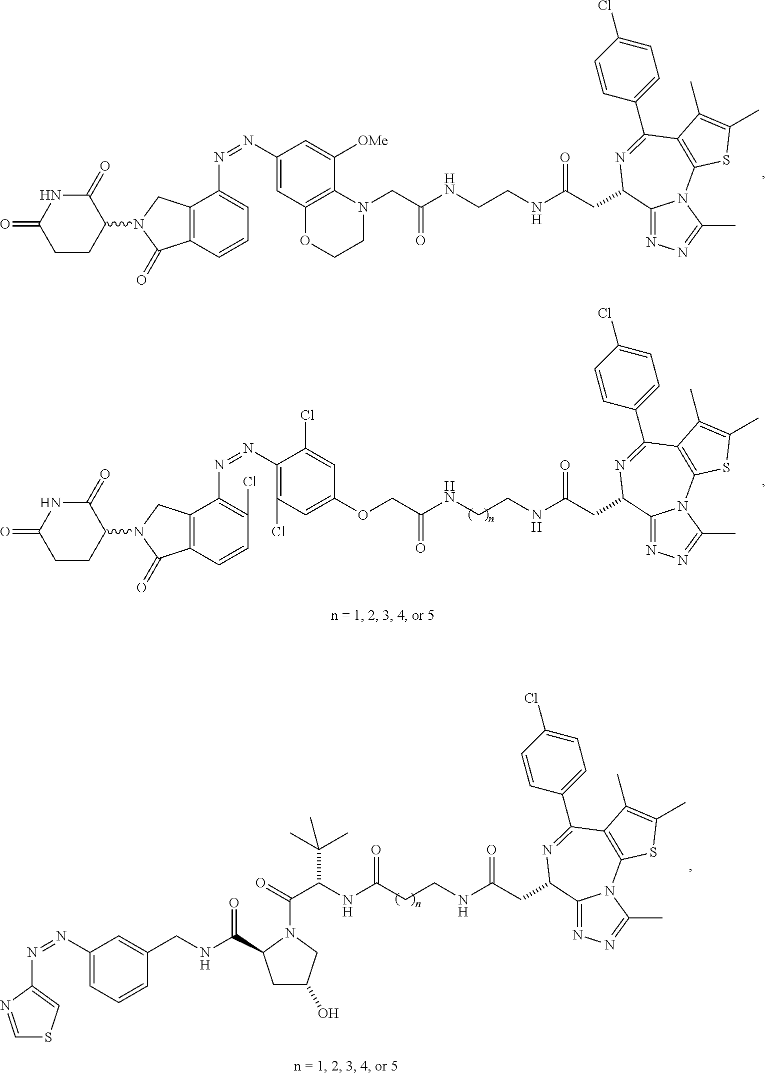

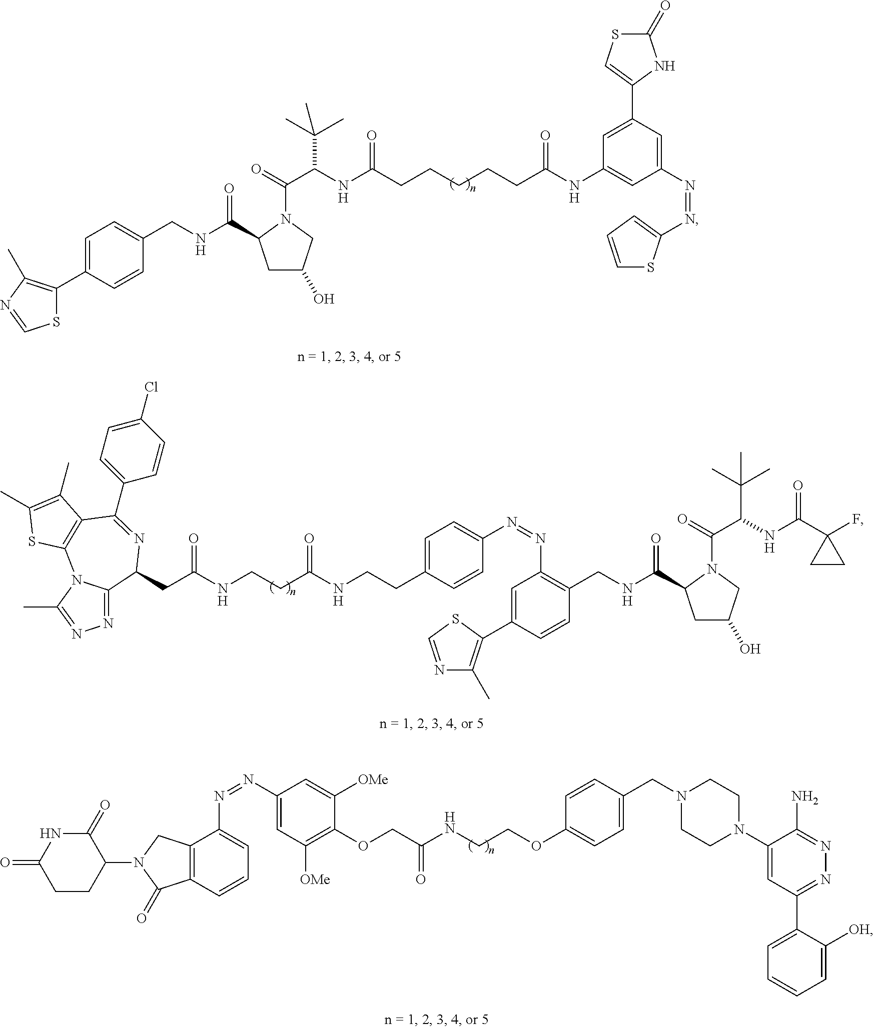

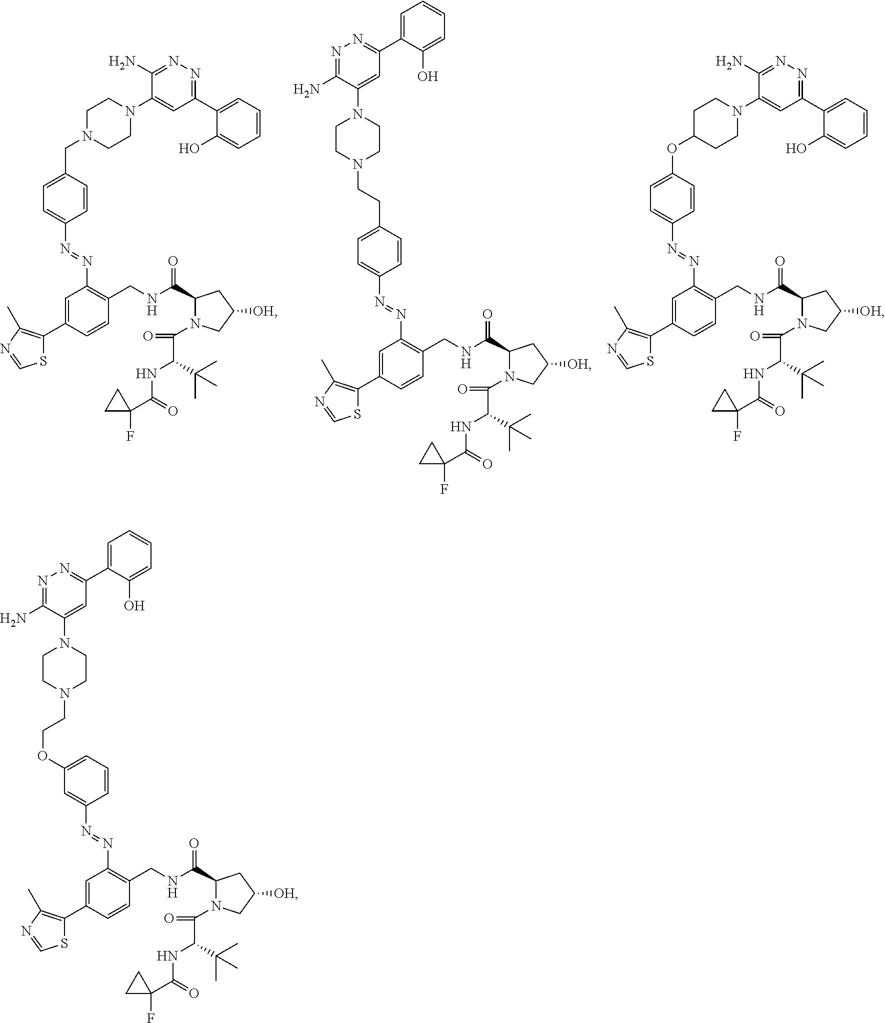

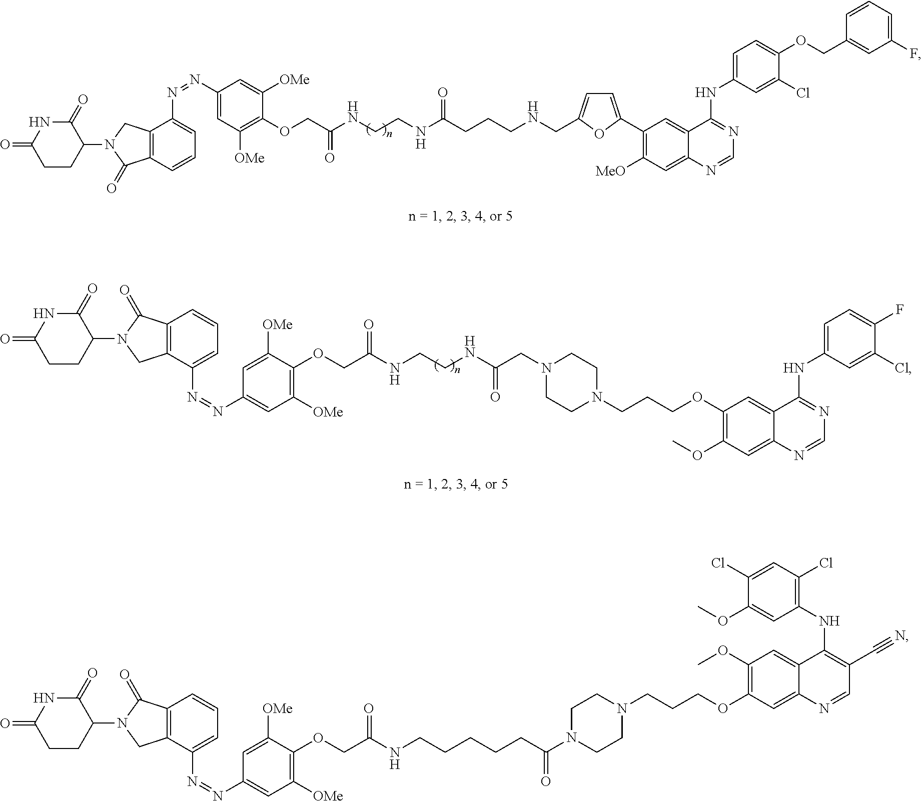

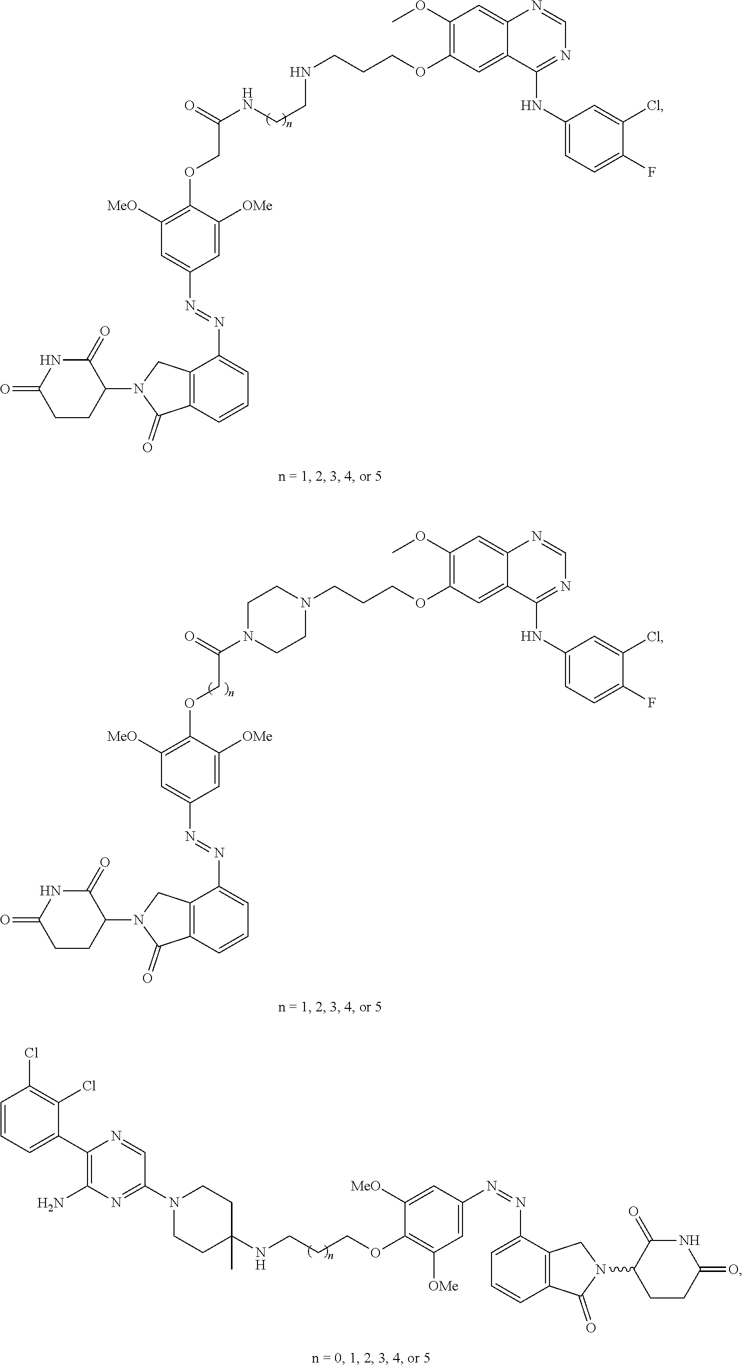

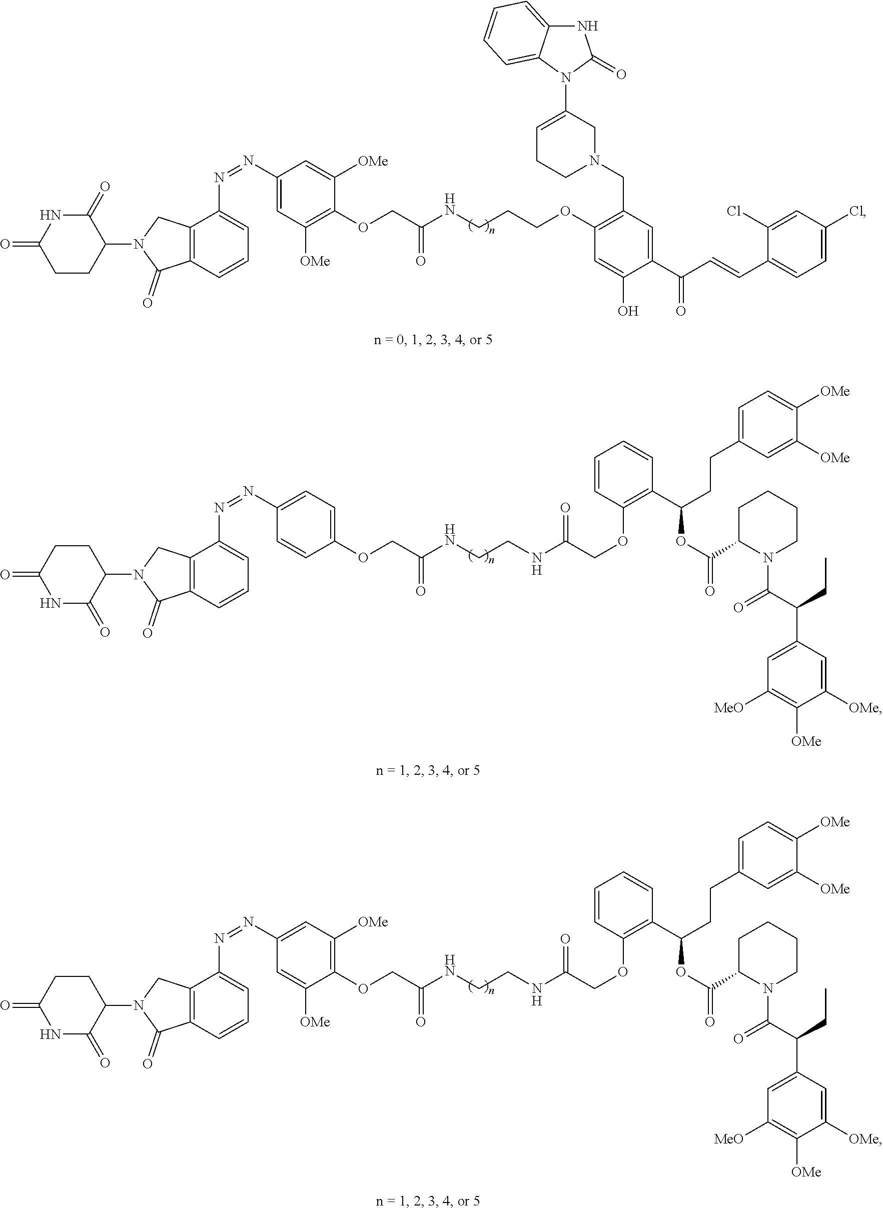

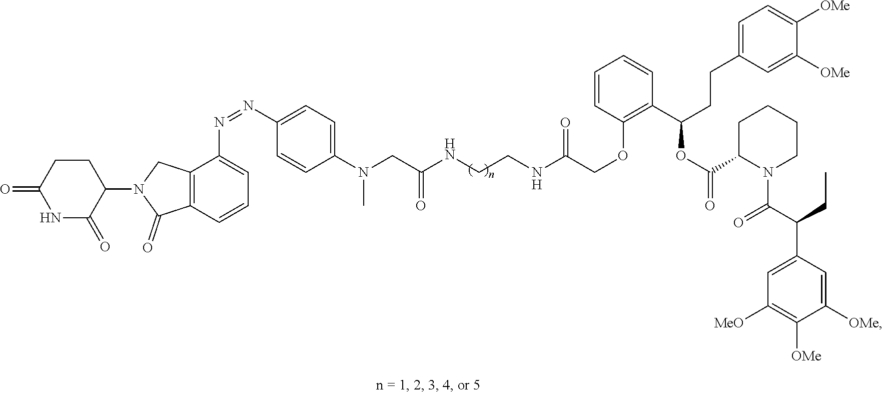

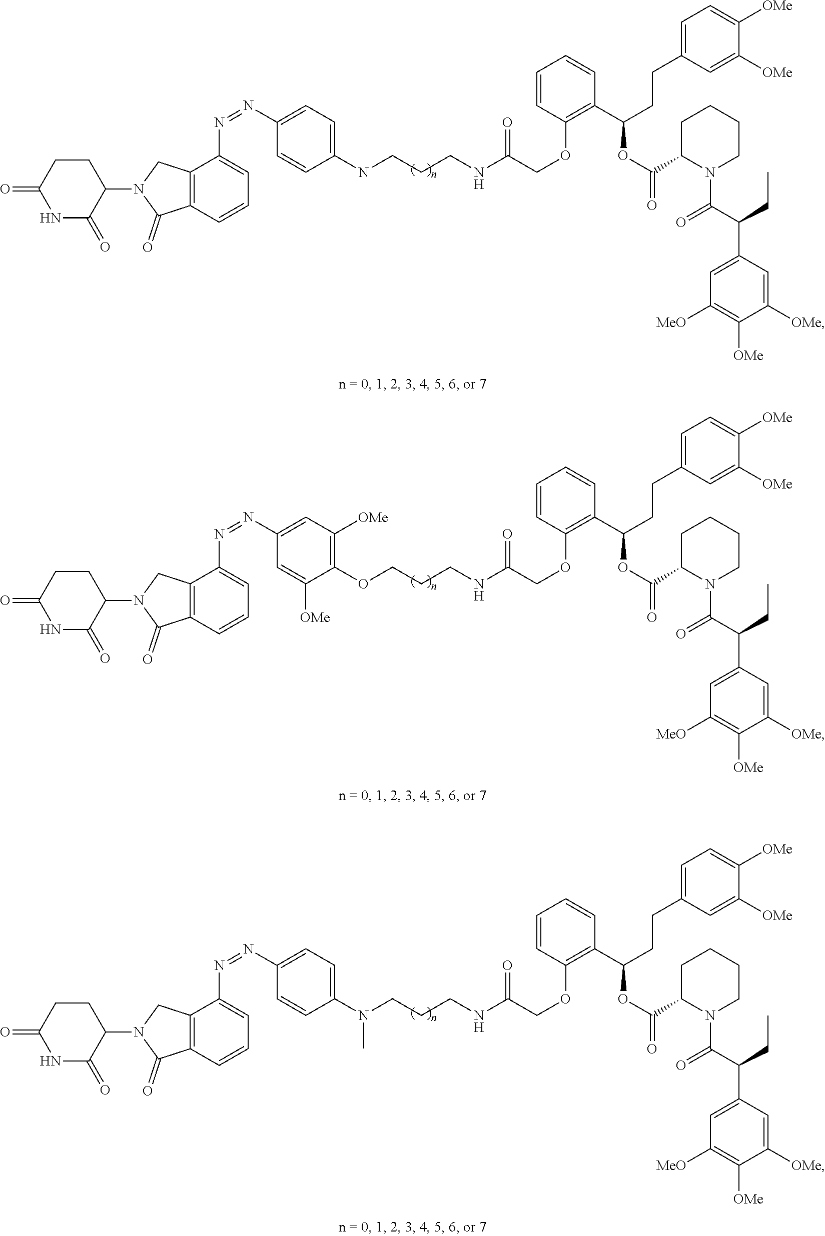

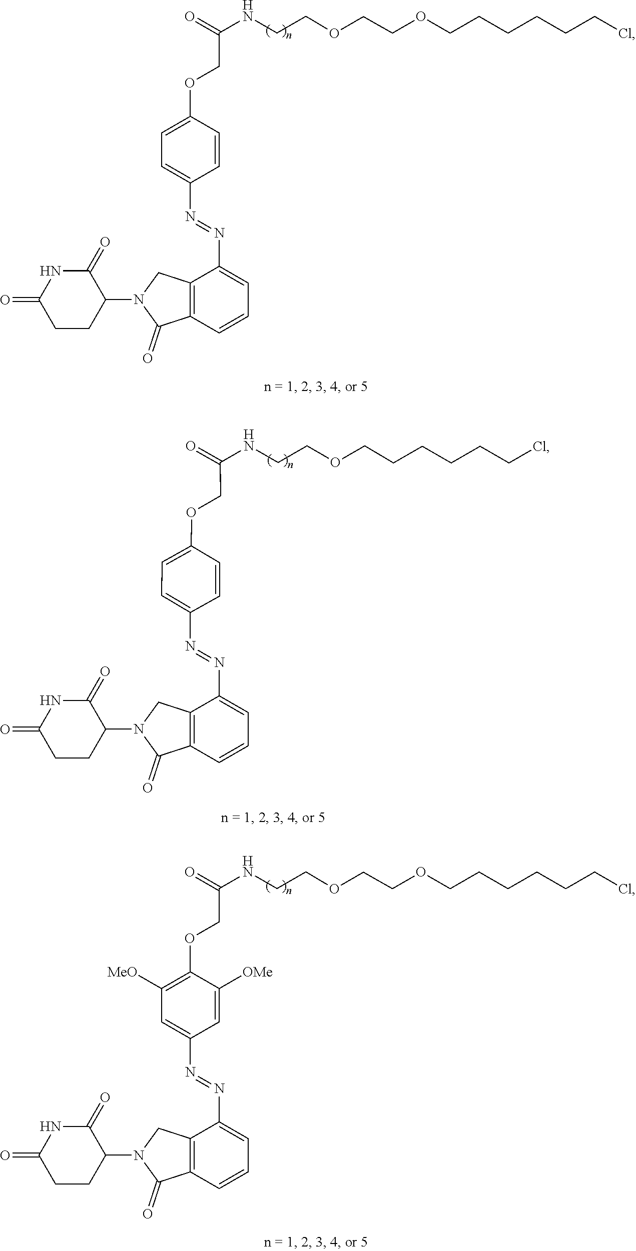

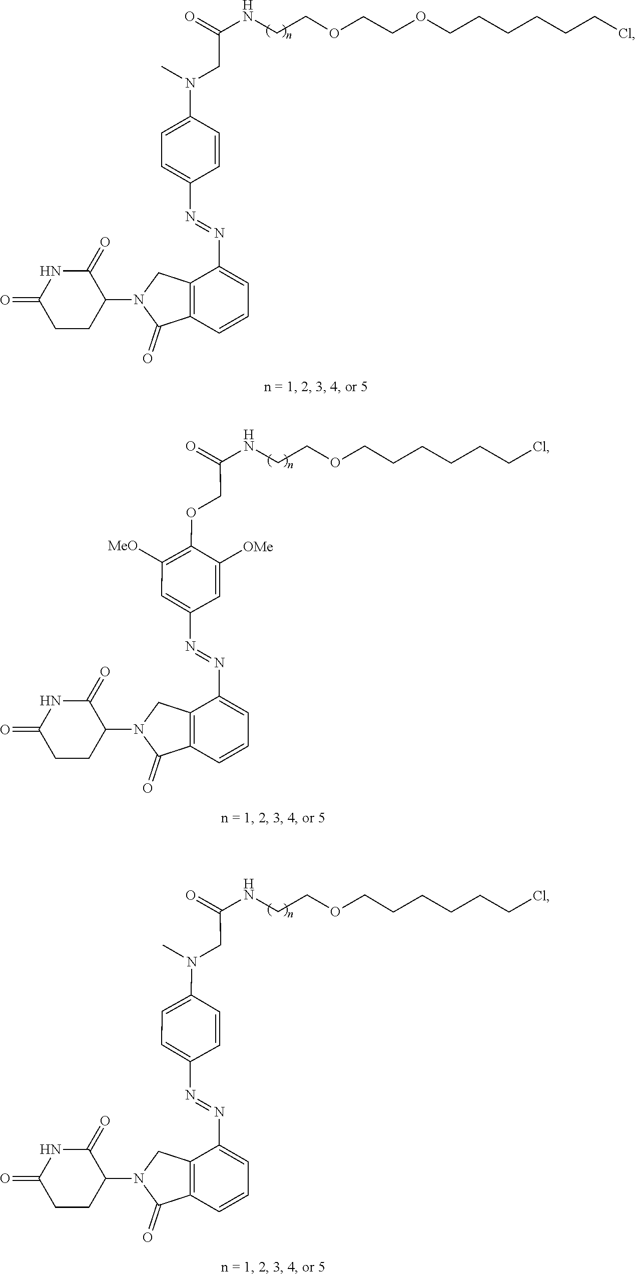

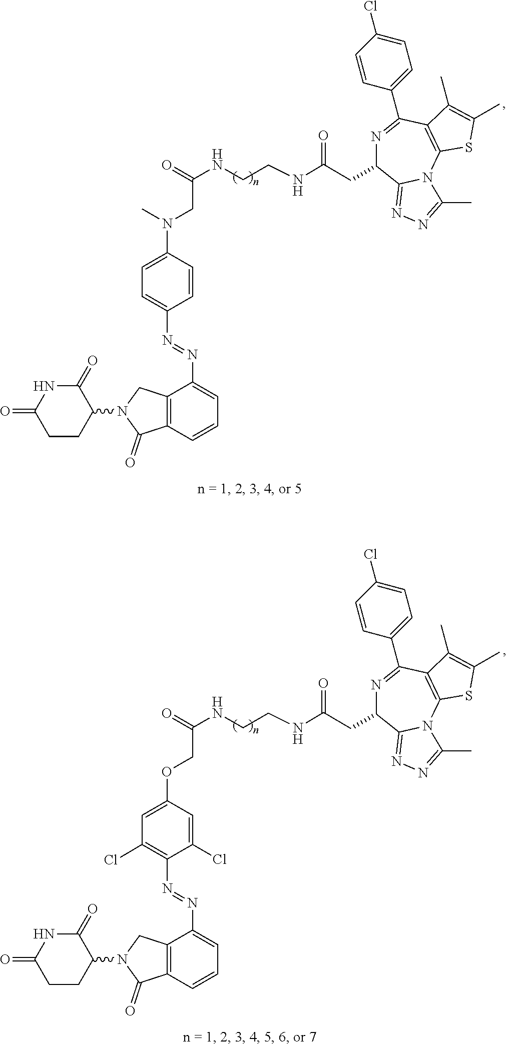

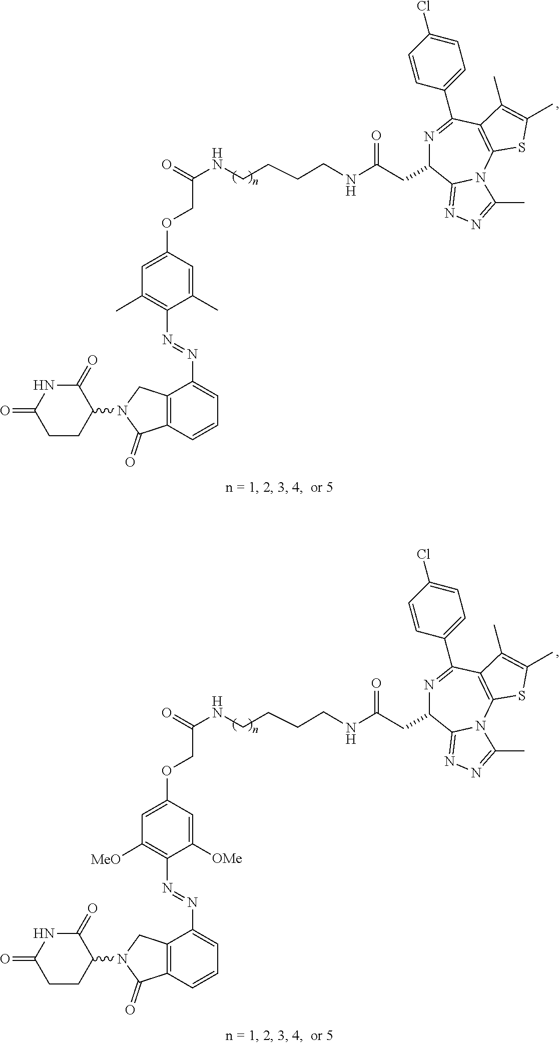

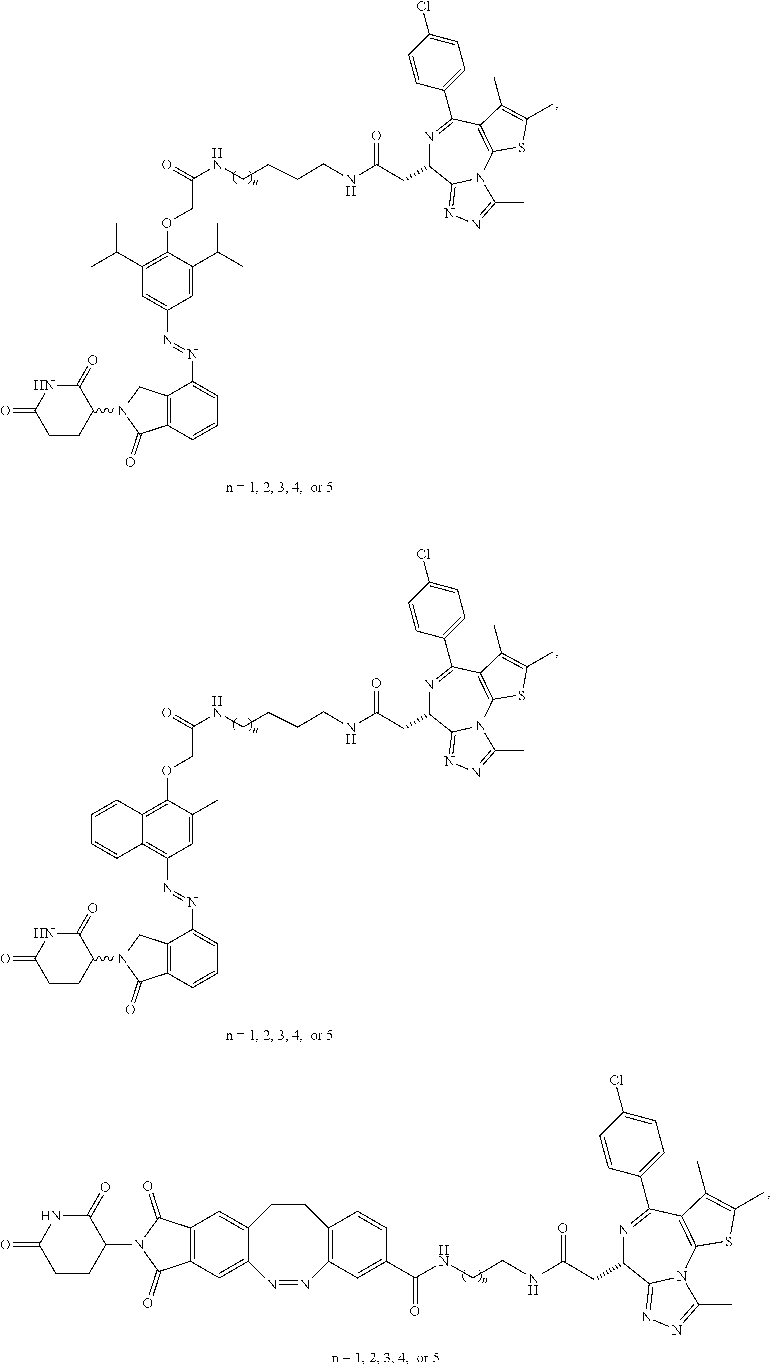

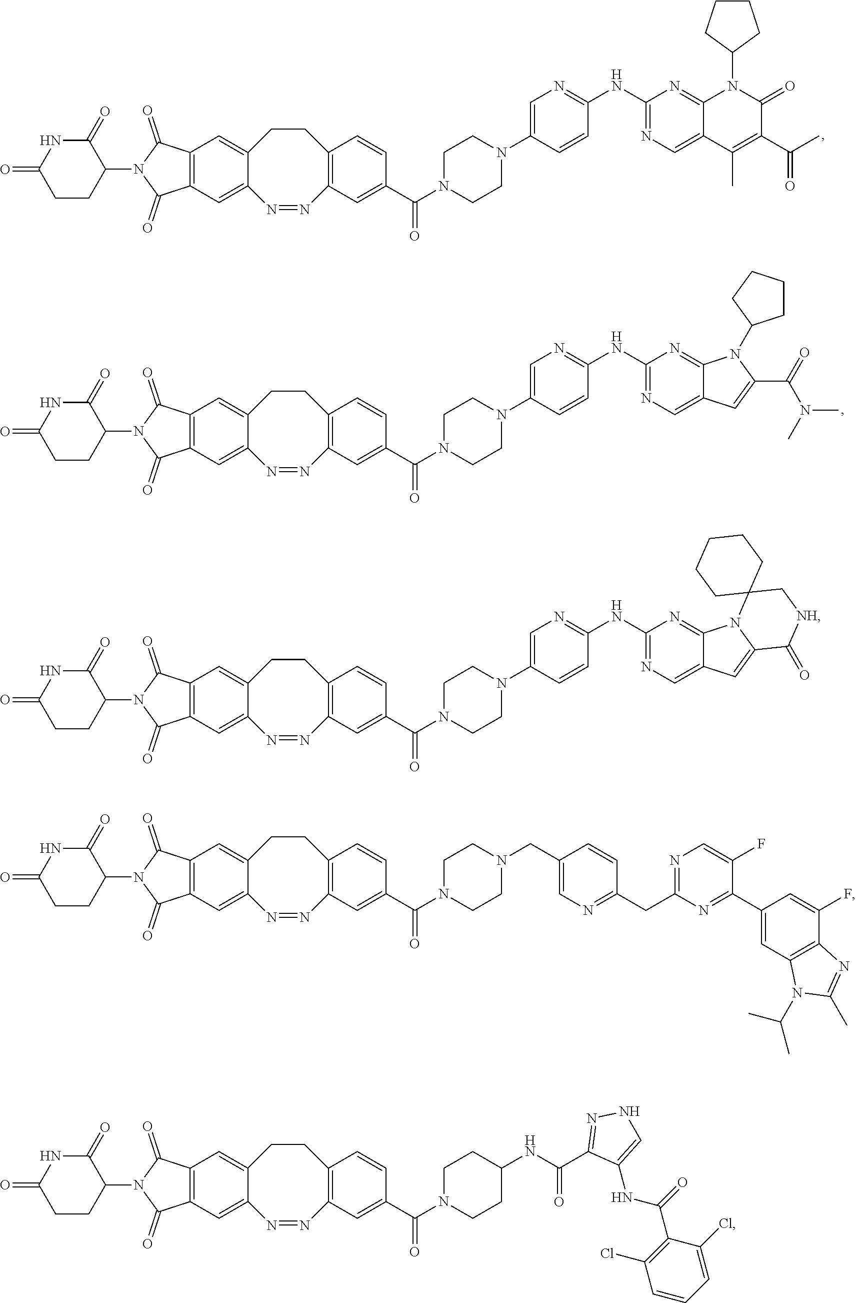

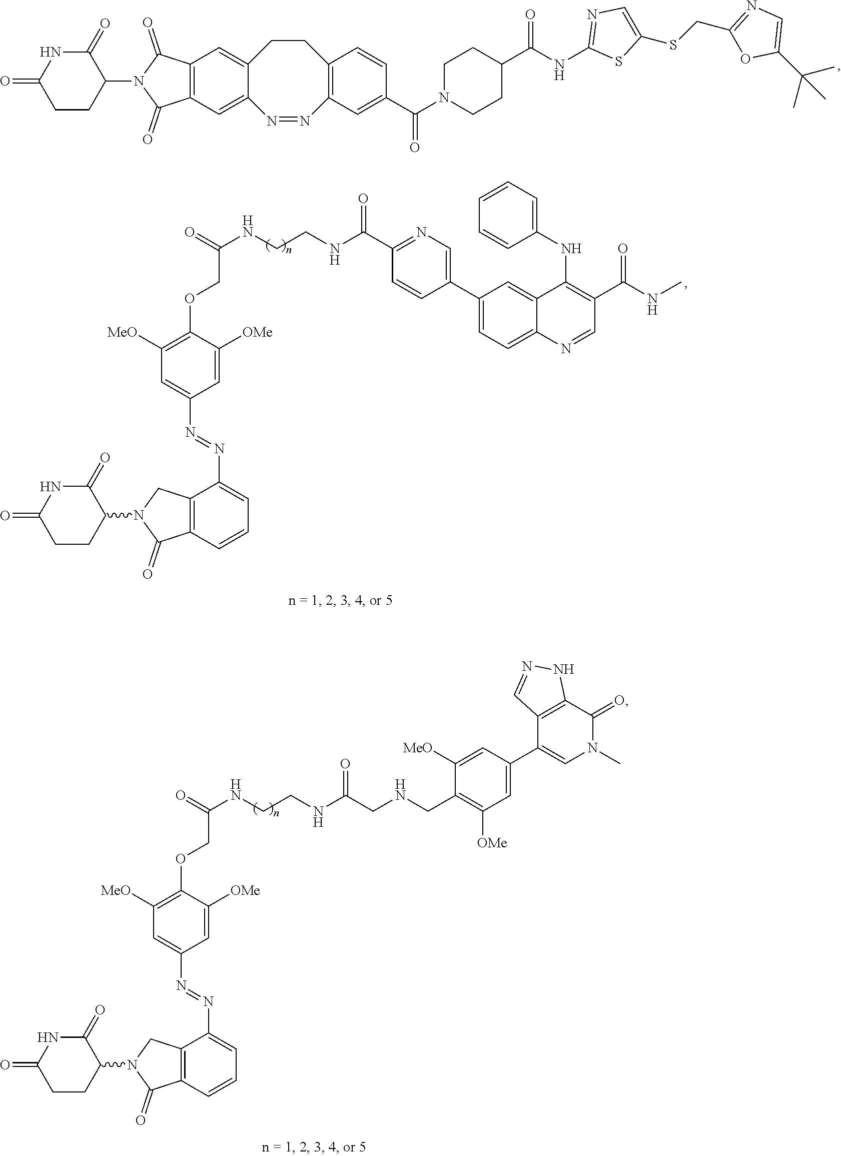

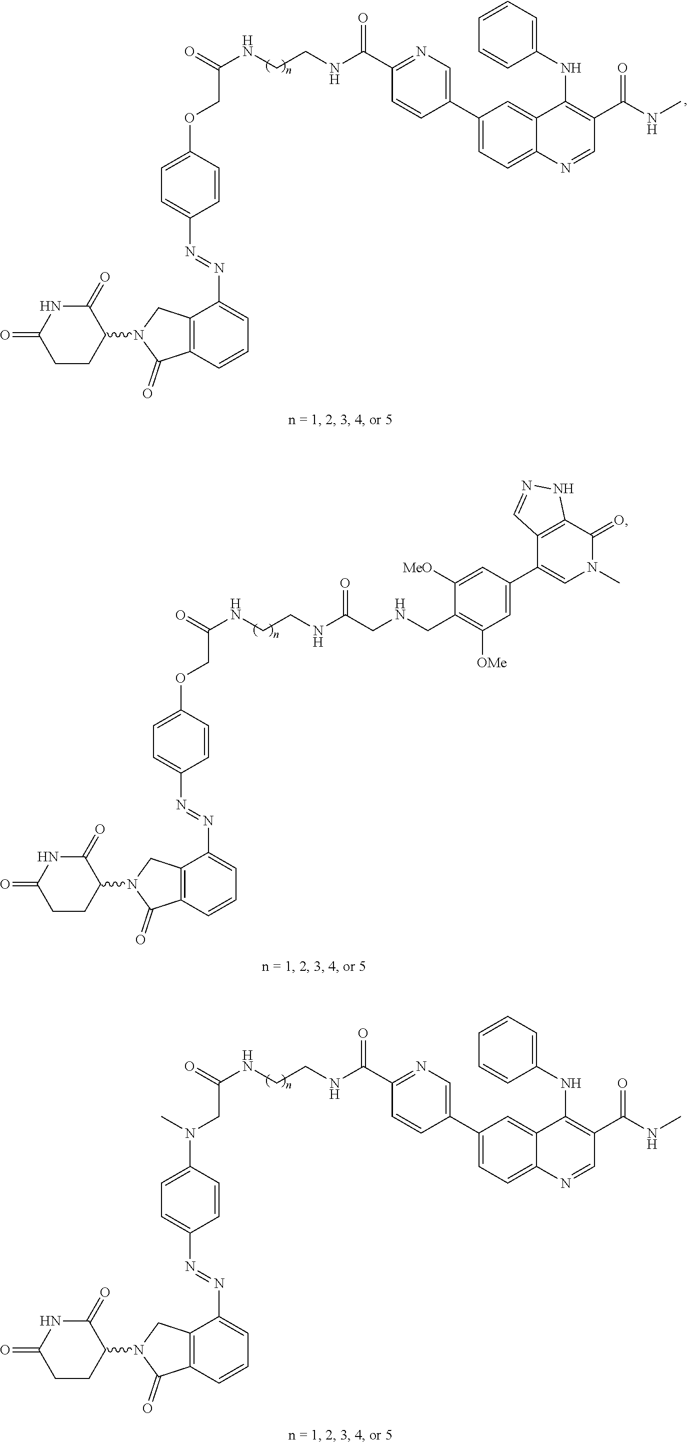

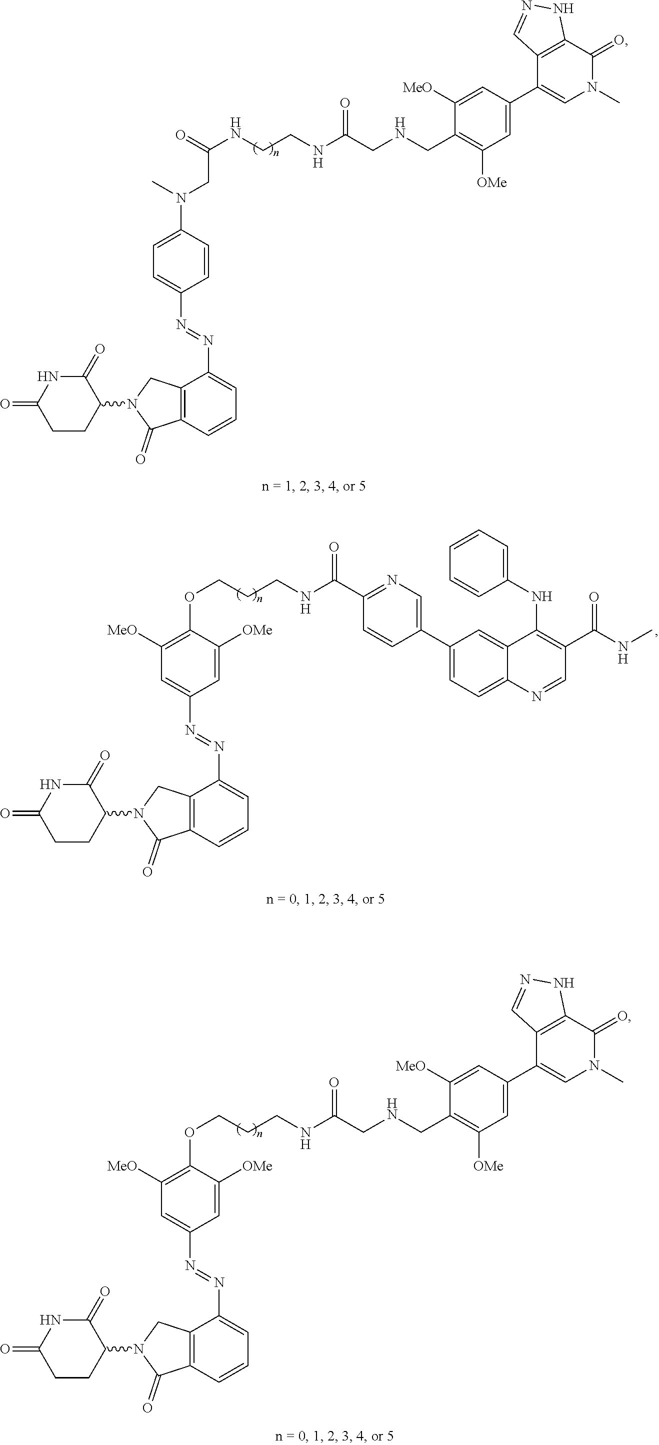

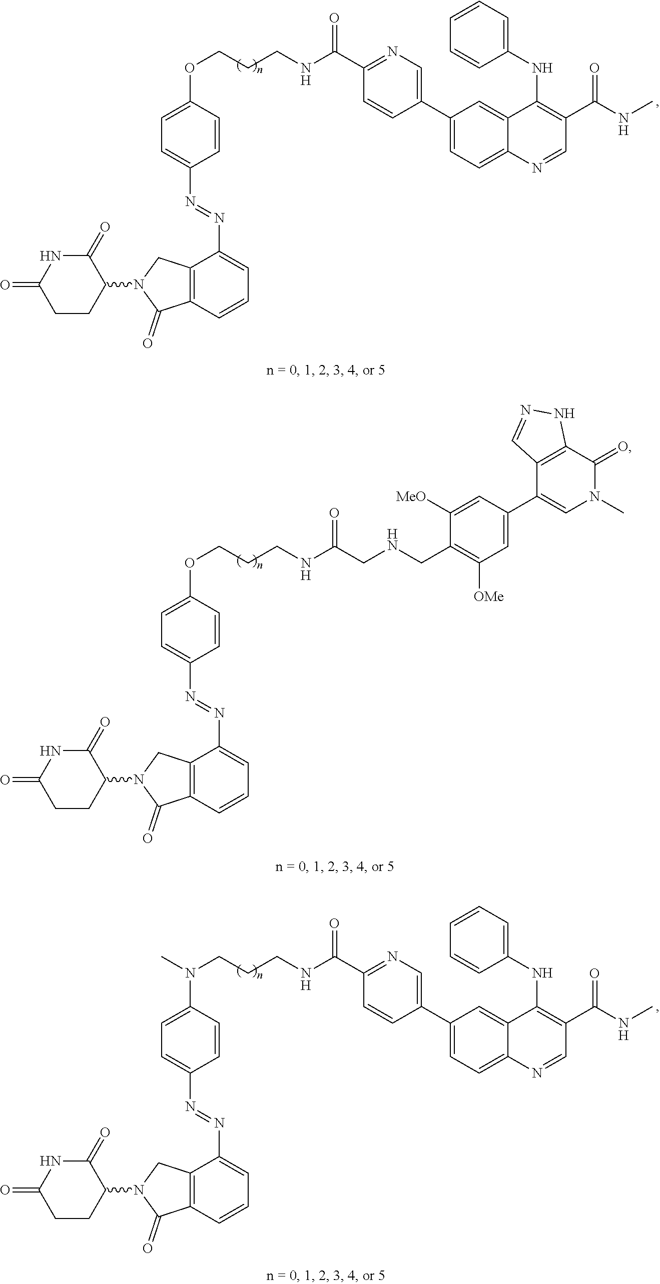

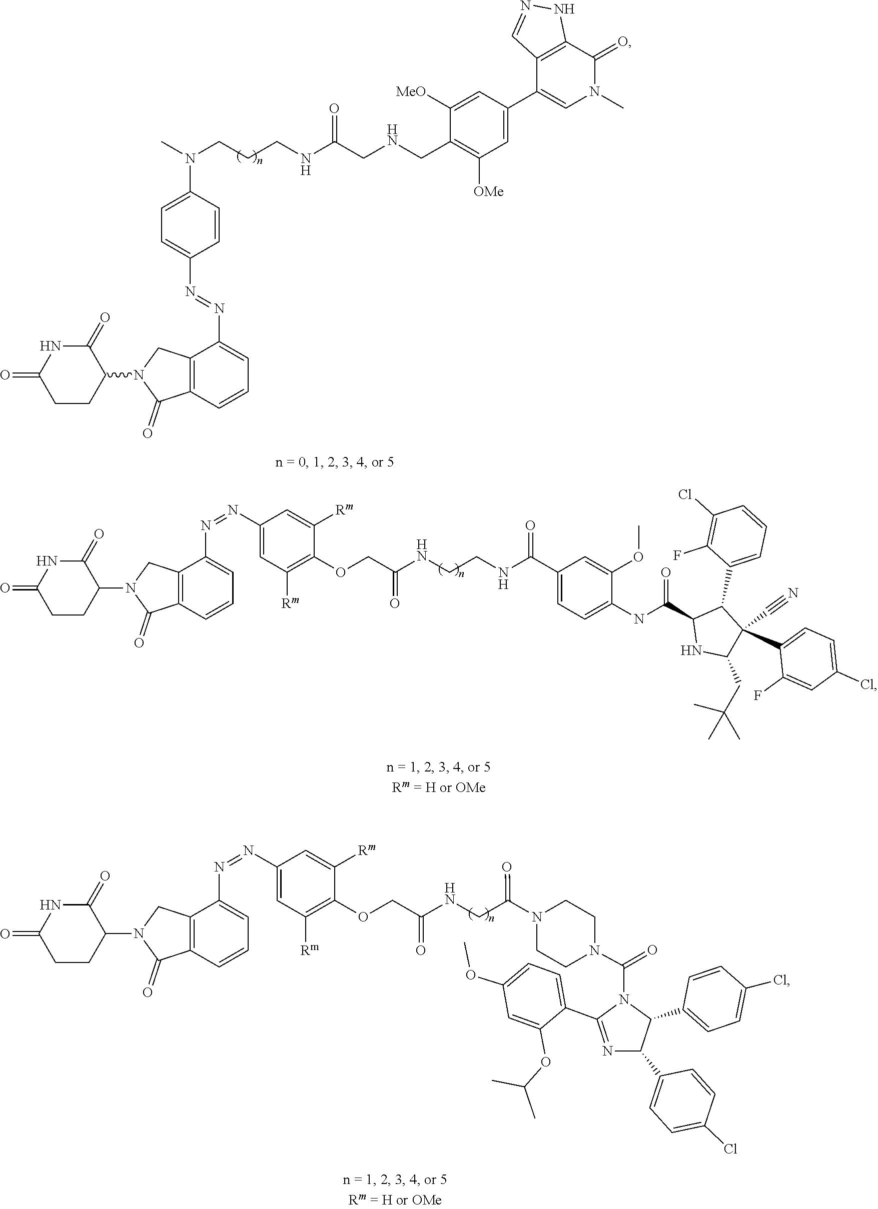

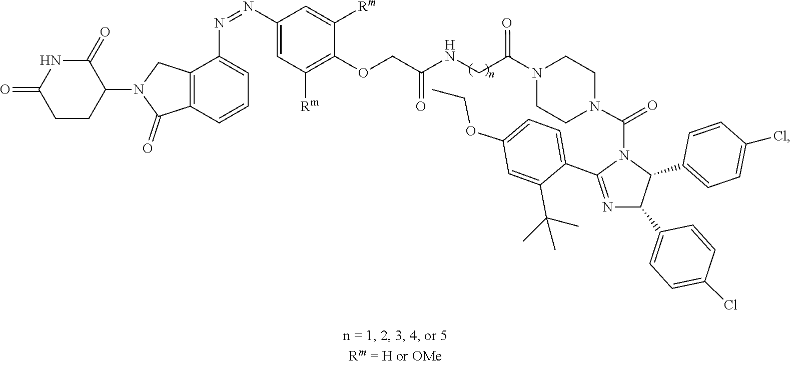

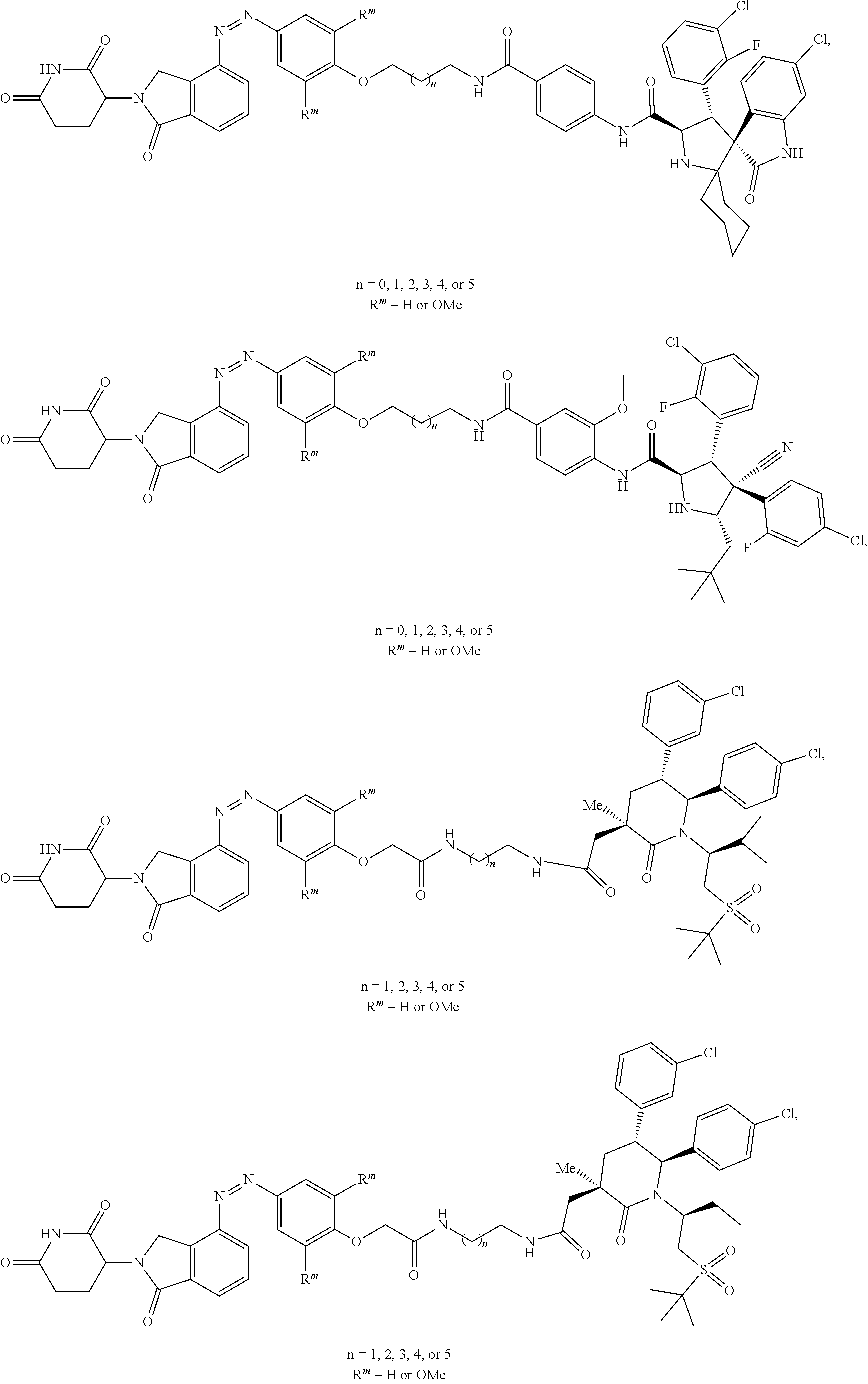

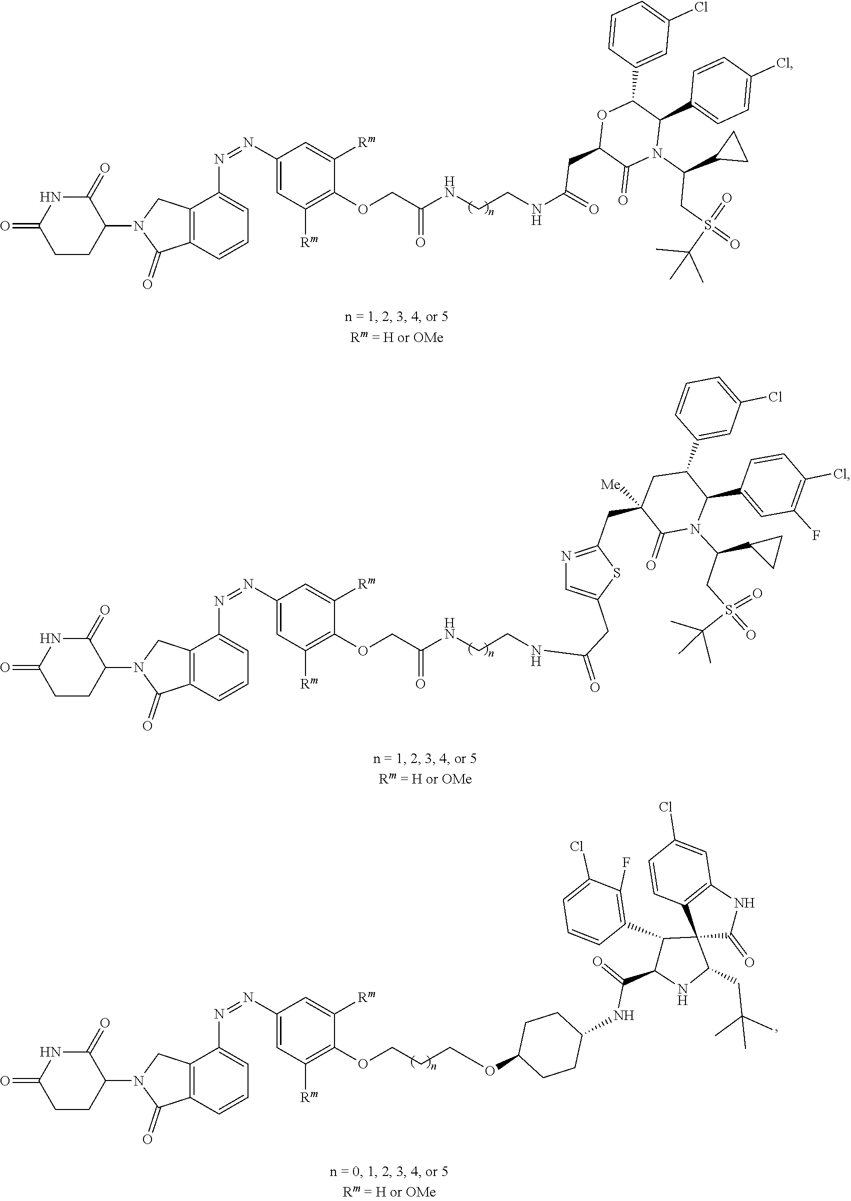

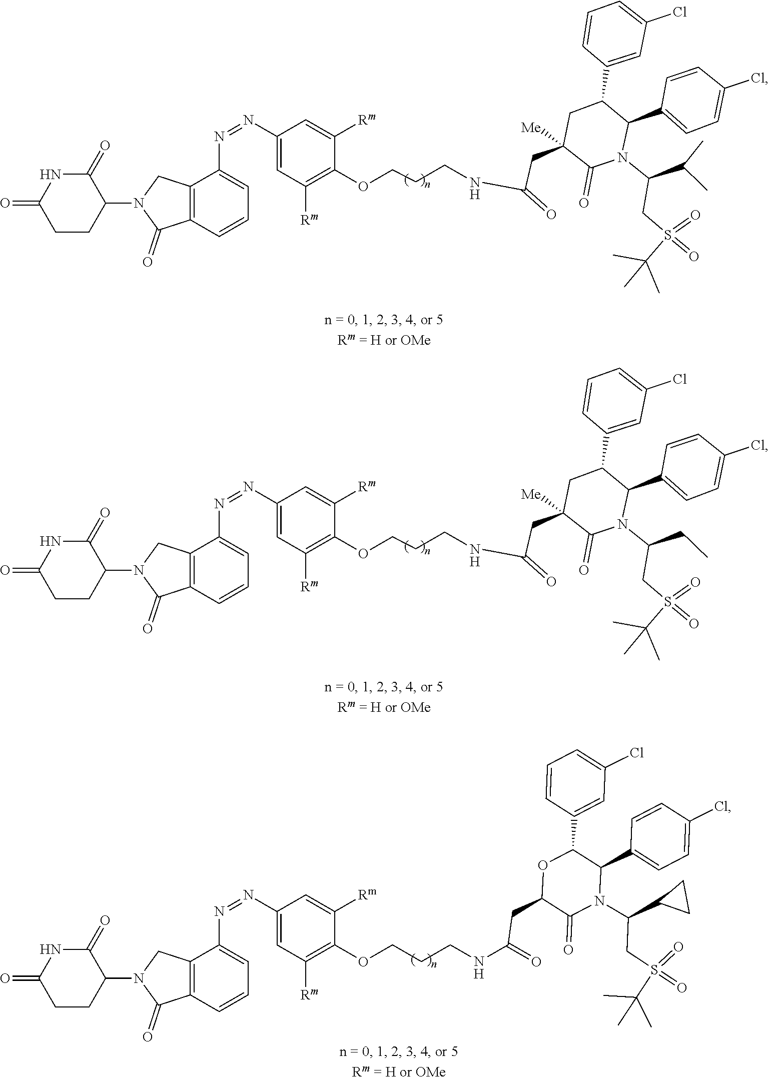

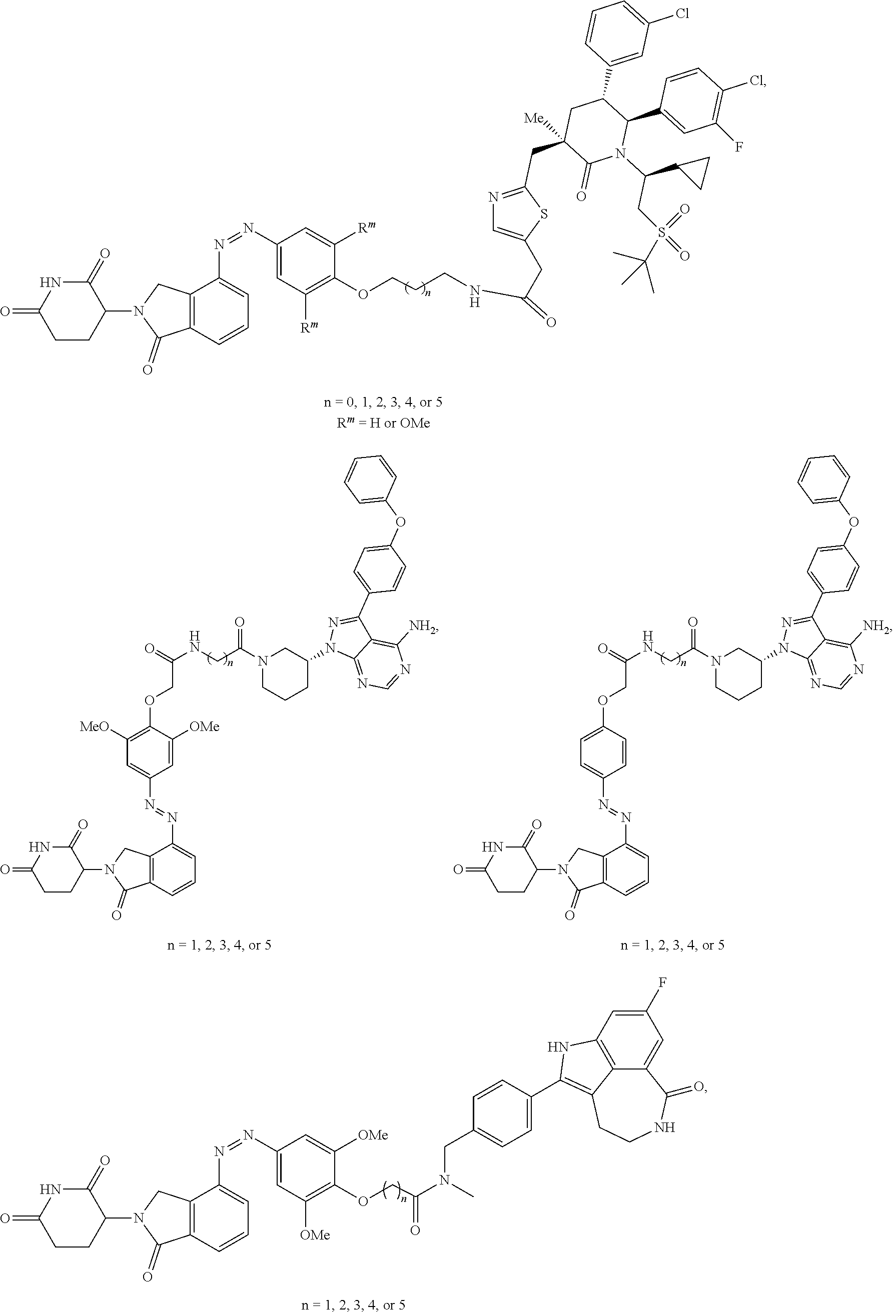

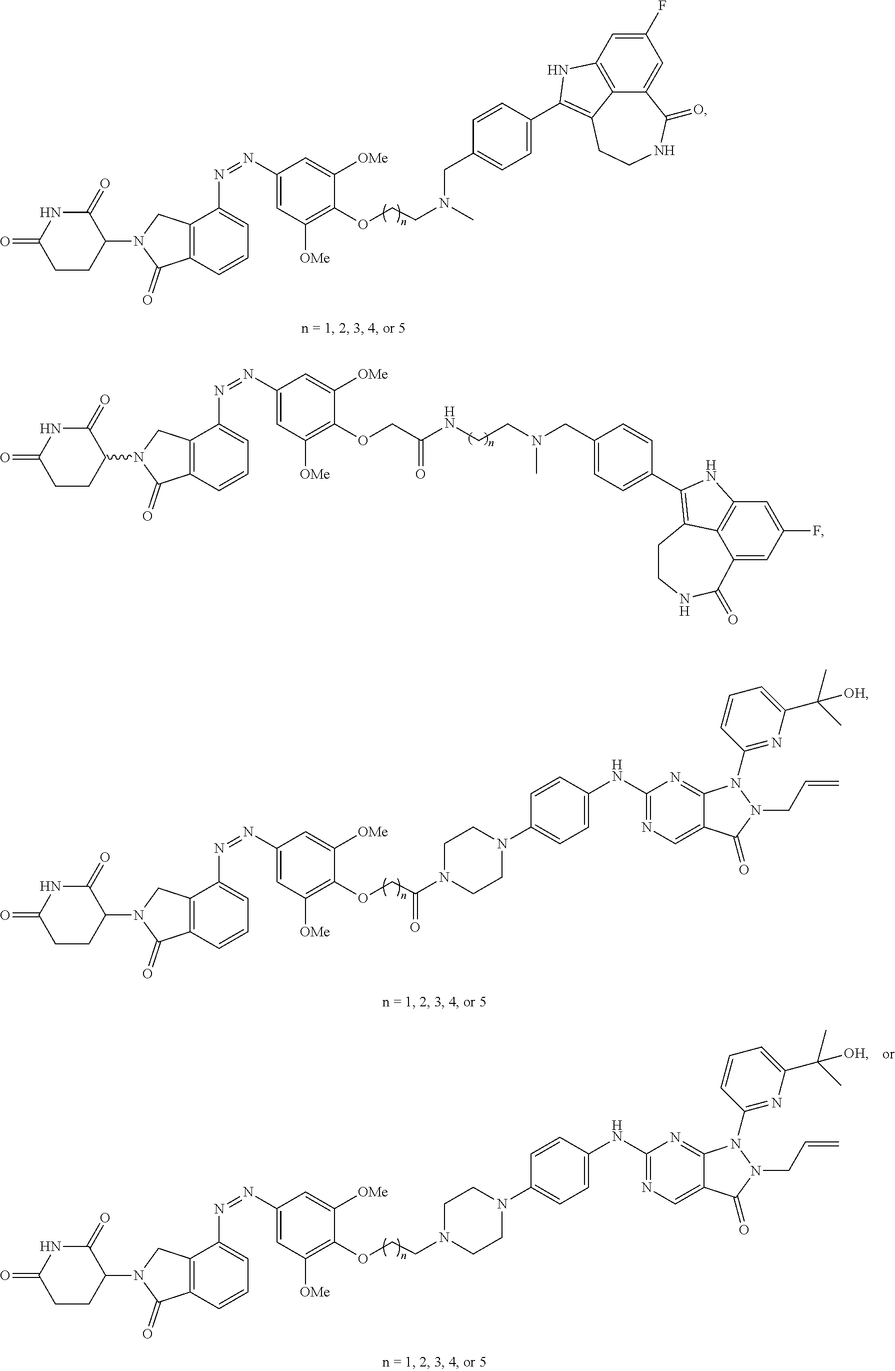

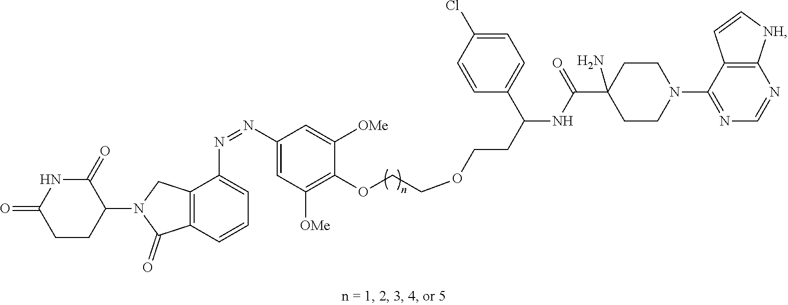

In various examples, a compound of the present disclosure is a salt (e.g., a hydrochloride salt, an N-oxide), a partial salt, a hydrate, a polymorph, a stereoisomer or a combination (e.g., a mixture) thereof. The compounds can have stereoisomers. In various examples, a compound is present as a racemic mixture, a single enantiomer, a single diastereomer, mixture of enantiomers, or mixture of diastereomers.

In an aspect, the present disclosure provides compositions comprising PHOTACs of the present disclosure. The compositions may further comprise one or more pharmaceutically acceptable carrier(s).

The compositions may include one or more pharmaceutically acceptable carrier(s). Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, such as, for example, sodium carboxymethyl cellulose, ethyl cellulose, and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example, glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2005) 21st Edition, Philadelphia, Pa. Lippincott Williams & Wilkins.

In various examples, a composition of the present disclosure comprises one or more pharmaceutically acceptable salt(s). Such a salt is a salt form of one or more of the compound(s) described herein, which may increase the solubility of the compound to promote dissolution and the bioavailability of the compounds. Non-limiting examples of pharmaceutically acceptable salts may include those derived from pharmaceutically acceptable inorganic or organic bases and acids, where applicable. Non-limiting examples of suitable salts may include those derived from alkali metals, such as, for example, potassium, sodium, and the like, alkaline earth metals, such as, for example, calcium, magnesium, and the like, ammonium salts, among numerous other acids and bases well known in the pharmaceutical art, and combinations thereof.

In various examples, a composition of the present disclosure comprises one or more pharmaceutically acceptable derivative(s). A pharmaceutically acceptable derivative is any pharmaceutically acceptable prodrug form (such as an ester, amide other prodrug group), which, upon administration to a patient or subject in need of treatment, provides directly or indirectly a PHOTAC of the present disclosure or an active metabolite of a PHOTAC of the present disclosure.

In an aspect, the present disclosure provides kits. In various examples, a kit comprises a pharmaceutical preparations containing any one or any combination of PHOTACs of the present disclosure. In an example, the instant disclosure includes a closed or sealed package that contains the pharmaceutical preparation. In certain embodiments, the package can comprise one or more closed or sealed vial(s), bottle(s), blister (bubble) pack(s), or any other suitable packaging for the sale, or distribution, or use of the pharmaceutical compounds and compositions comprising them. The printed material can include printed information. The printed information may be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of a subject having cancer and/or other diseases and/or any disorder associated with cancer and/or other diseases. In embodiments the product includes a label describing the contents of the container and providing indications and/or instructions regarding use of the contents of the container to treat a subject having any cancer and/or other diseases.

In an aspect, the present disclosure provides methods of using one or more PHOTAC(s) and/or one or more composition(s) comprising one or more PHOTAC(s). PHOTACs of the present disclosure are suitable for use in methods to treat diseases. Examples of diseases include, but are not limited to, cancers (e.g., leukemia, lung cancer, dermatological cancer, premalignant lesions of the upper digestive tract, malignancies of the prostate, malignancies of the brain, malignancies of the breast, and the like, and combinations thereof) and/or other diseases (e.g., infectious diseases, inflammatory diseases, immune disorders, sleep disorders, neurodegenerative disorders, and the like, and combinations thereof) and methods to induce protein degradation. In various examples, one or more PHOTAC(s) of the present disclosure is/are used to treat one or more diseas(es), such as, for example, cancer, other various disease(s), or a combination thereof, and/or induce selective degradation of a target protein. A method may be carried out in combination with one or more known therapy(ies).

Various cancers may be treated via a method of the present disclosure. Non-limiting examples of cancers include leukemia, lung cancer (e.g., non-small cell lung cancer), dermatological cancers, premalignant lesions of the upper digestive tract, malignancies of the prostate, malignancies of the brain, malignancies of the breast, solid tumors, and the like, and combinations thereof.

Various cancers that may be treated by PHOTACs of the present disclosure either alone or in combination with at least one additional anti-cancer agent include, but are not limited to, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, but are not limited to, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML, and the like, and combinations thereof.

Various other diseases may be treated via a method of the present disclosure. Other diseases include, but are not limited to, infectious diseases, inflammatory diseases, immune disorders, sleep disorders, neurodegenerative disorders, and the like, and combinations thereof. Non-limiting examples of other diseases include asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome, Alzheimer's disease, amyotrophic lateral sclerosis (Lou Gehrig's disease), anorexia nervosa, anxiety disorder, atherosclerosis, attention deficit hyperactivity disorder, autism, bipolar disorder, chronic fatigue syndrome, chronic obstructive pulmonary disease, Crohn's disease, coronary heart disease, dementia, depression, diabetes mellitus type 1, diabetes mellitus type 2, epilepsy, Guillain-Barre syndrome, irritable bowel syndrome, lupus, metabolic syndrome, multiple sclerosis, myocardial infarction, obesity, obsessive-compulsive disorder, panic disorder, Parkinson's disease, psoriasis, rheumatoid arthritis, sarcoidosis, schizophrenia, stroke, thromboangiitis obliterans, Tourette's syndrome, vasculitis, aceruloplasminemia, achondrogenesis type II, achondroplasia, acrocephaly, gaucher disease type 2, acute intermittent porphyria, canavan disease, adenomatous polyposis coli, ALA dehydratase deficiency, adenylosuccinate lyase deficiency, adrenogenital syndrome, Adrenoleukodystrophy, ALA-D porphyria, alkaptonuria, Alexander disease, alkaptonuric ochronosis, alpha 1-antitrypsin deficiency, alpha-1 proteinase inhibitor, emphysema, amyotrophic lateral sclerosis Alstrom syndrome, amelogenesis imperfecta, Anderson-Fabry disease, androgen insensitivity syndrome, anemia angiokeratoma corporis diffusum, angiomatosis retinae (von Hippel-Lindau disease), apert syndrome, arachnodactyly (Marfan syndrome), stickler syndrome, arthrochalasis multiplex congenital (Ehlers-Danlos syndrome arthrochalasia type) ataxia telangiectasia, Rett syndrome, primary pulmonary hypertension, sandhoff disease, neurofibromatosis type II, Beare-Stevenson cutis gyrata syndrome, Mediterranean fever, familial, Benjamin syndrome, beta-thalassemia, bilateral acoustic neurofibromatosis (neurofibromatosis type II), factor V leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, brittle bone disease (osteogenesis imperfecta), broad thumb-hallux syndrome (Rubinstein-Taybi syndrome), bronze diabetes/bronzed cirrhosis (hemochromatosis), bulb ospinal muscular atrophy (Kennedy's disease), Burger-Grutz syndrome (lipoprotein lipase deficiency), CGD chronic granulomatous disorder, campomelic dysplasia, biotinidase deficiency, cardiomyopathy (Noonan syndrome), Cri du chat, CAVD (congenital absence of the vas deferens), caylor cardiofacial syndrome (CBAVD), CEP (congenital erythropoietic porphyria), cystic fibrosis, congenital hypothyroidism, chondrodystrophy syndrome (achondroplasia), otospondylomegaepiphyseal dysplasia, Lesch-Nyhan syndrome, galactosemia, Ehlers-Danlos syndrome, thanatophoric dysplasia, Coffin-Lowry syndrome, Cockayne syndrome, (familial adenomatous polyposis), congenital erythropoietic porphyria, congenital heart disease, methemoglobinemia/congenital methaemoglobinaemia, achondroplasia, X-linked sideroblastic anemia, connective tissue disease, conotruncal anomaly face syndrome, Cooley's Anemia (beta-thalassemia), copper storage disease (Wilson's disease), copper transport disease (Menkes disease), hereditary coproporphyria, Cowden syndrome, craniofacial dysarthrosis (Crouzon syndrome), Creutzfeldt-Jakob disease (prion disease), Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, degenerative nerve diseases including de Grouchy syndrome and Dejerine-Sottas syndrome, developmental disabilities, distal spinal muscular atrophy, type V, androgen insensitivity syndrome, diffuse globoid body sclerosis (Krabbe disease), Di George's syndrome, dihydrotestosterone receptor deficiency, androgen insensitivity syndrome, Down syndrome, dwarfism, erythropoietic protoporphyria erythroid 5-aminolevulinate synthetase deficiency, erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), fibrocystic disease of the pancreas, fragile X syndrome, galactosemia, genetic brain disorders, giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), hyperandrogenism, hypochondroplasia, hypochromic anemia, immune system disorders, including X-linked severe combined immunodeficiency, Insley-Astley syndrome, Jackson-Weiss syndrome, Joubert syndrome, Lesch-Nyhan syndrome, kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, metabolic disorders, including Kniest dysplasia, Marfan syndrome, movement disorders, Mowat-Wilson syndrome, cystic fibrosis, Muenke syndrome, multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, skin pigmentation disorders, Smith-Lemli-Opitz syndrome, South-African genetic porphyria (variegate porphyria), infantile-onset ascending hereditary spastic paralysis, speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), vision impairment and blindness (Alstrom syndrome), Vrolik disease, Waardenburg syndrome, Warburg Sjo Fledelius Syndrome, Weissenbacher-Zweymuller syndrome, Wolf-Hirschhorn syndrome, Wolff Periodic disease, and Xeroderma pigmentosum, and the like, and combinations thereof.

In various examples, one or more PHOTAC(s) and/or one or more composition(s) comprising one or more PHOTAC(s) described herein can be administered to a subject in need of treatment using any known method and route, including, but not limited to, oral, parenteral, subcutaneous, intraperitoneal, intrapulmonary, intranasal and intracranial injections, and the like. Parenteral infusions include intramuscular, intravenous, intraarterial, intraperitoneal, subcutaneous administration, and the like. Topical and/or transdermal administrations are also contemplated by present disclosoure.

A method can be carried out in a subject in need of treatment who has been diagnosed with or is suspected of having a disease (e.g., cancer and/or other disease(s)) (i.e., therapeutic use). A method can also be carried out in a subject who have a relapse or a high risk of relapse after being treated for cancer and/or other disease(s).

A method of the present disclosure may be combined with various other treatments, such as, for example, other agents used to treat cancer. Non-limiting examples of agents to treat cancer include everolimus, trabectedin, abraxane, TLK 286, AV-299, DN-101, pazopanib, GSK690693, RTA 744, ON 0910. Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR.sub.1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]-benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and the like, and combinations thereof.

A subject in need of treatment may be a human or non-human mammal or other animal. Non-limiting examples of non-human mammals include cows, pigs, mice, rats, rabbits, cats, dogs, or other agricultural mammals, pet, or service animals, and the like.

PHOTACs can be irradiated with electromagnetic radiation (e.g., having one or more wavelength(s) from or between 300 and 1500 nm) for which duration, intensity, and exposure region, cell, tissue, tumor zone, organism or other region of interest may be varied. PHOTACs can also be irradiated with electromagnetic radiation before being applied to the subject, alone, or as a pharmaceutical formulation. Light sources and delivery methods include, but are not limited to, lamps, light-emitting diodes (LEDs), organic LEDs (OLEDs), lasers, monochromators and sun light and may be coupled with methods for light delivery and focusing, including endoscopic techniques and fibre optic cables, optical table setups, microscopy methods, implantaple LEDs and OLEDs, upconverting nanoparticles, photosensitizers (including triplet fusion upconverting photosensitizers, FRET sensitizers, two photon-antennae). Other established methods in photodynamic therapy or light therapy, such as extracorporeal photopheresis methods can be used to irradiate the PHOTACs. PHOTACs may be activated and deactivated by transdermal irradiation, implantable light sources or other temporary or permanently inserted light sources in the treated subject. LEDs and OLEDs may be wired or wirelessly powered or powered by batteries.

In various examples, a method to induce selective degradation of a target protein comprises: i) contacting a cell (e.g., a cell in a subject in need of treatment) with a PHOTAC and/or a composition of the present disclosure, where the PHOTAC is in a trans conformation and PHOTAC binds to an E3 ligase; and ii) exposing the cell or a portion thereof to electromagnetic radiation (e.g., light having a wavelength of 375-405 nm (such as, for example, 390 nm)), where the exposing induces a conformational change in the PHOTAC and the PHOTAC binds to the target protein.

In various examples, a method to induce selective degradation of a target protein comprises: i) contacting a cell (e.g., a cell in a subject in need of treatment) with a PHOTAC and/or a composition of the present disclosure, where the PHOTAC is in a trans conformation and the PHOTAC binds to the target protein; and ii) exposing the cell or a portion thereof to electromagnetic radiation (e.g., light having a wavelength of 375-405 nm (such as, for example, 390 nm)), where the exposing induces a conformational change in the PHOTAC and PHOTAC binds to an E3 ligase.

In various examples, a method to induce selective degradation of a target protein comprises: i) contacting a cell (e.g., a cell in a subject in need of treatment) with a PHOTAC and/or a composition the present disclosure, where the PHOTAC is in a cis conformation and the PHOTAC binds to the target protein and E3 ligase; and ii) optionally, exposing the cell or a portion thereof to electromagnetic radiation (e.g., light having a wavelength of 485-515 nm (such as, for example, 500 nm)), where the exposing induces a conformational change in the PHOTAC.

In various examples, a method of the present disclosure for treating a disease (e.g., cancer and/or other disease(s)) comprises: i) administering to a subject in need of treatment a composition of the present disclosure, where the PHOTAC of the current disclosure is in a trans conformation and binds to an E3 ligase; and ii) exposing the subject in need of treatment or a portion thereof to electromagnetic radiation (e.g., light having a wavelength of 375-405 nm (such as, for example, 390 nm)), where the exposing induces a conformational change in the PHOTAC and the PHOTAC binds to a target protein.

In various examples, a method of the present disclosure for treating a disease (e.g., cancer and/or other disease(s)) comprises: i) administering to a subject in need of treatment a composition of the present disclosure, where the PHOTAC is in a trans conformation and binds to a target protein; and ii) exposing the subject in need of treatment or a portion thereof to electromagnetic radiation (e.g., light having a wavelength of 375-405 nm (such as, for example, 390 nm)), where the exposing induces a conformational change in the PHOTAC and the PHOTAC binds to an E3 ligase.

In various examples, a method of the present disclosure for treating a disease (e.g., cancer and/or other disease(s)) comprises: i) administering to a subject in need of treatment a composition of the present disclosure, where the PHOTAC of the present disclosure is in a cis conformation and binds to a target protein and an E3 ligase; and ii) exposing the subject in need of treatment or a portion thereof to electromagnetic radiation (e.g., light having a wavelength of 485-515 nm (such as, for example, 500 nm)), wherein the exposing induces a conformational change in the PHOTAC.

In various examples, a method of the present disclosure comprises contacting a cell with a PHOTAC of the present disclosure and/or administering to a subject in need of treatment a composition of the present disclosure, where the PHOTAC is in a relaxed state and then exposed to electromagnetic radiation to isomerize the PHOTAC to an unstable state. The PHOTAC may be isomerized one or more additional time(s). Alternatively, in various other examples, the method of the present disclosure comprises contacting a cell with a PHOTAC of the present disclosure and/or administering to a subject in need of treatment a composition of the present disclosure, where the PHOTAC is in an unstable state, and then it either isomerizes over a period of time to its relaxed state or is exposed to electromagnetic radiation such that it isomerizes to its relaxed state. As before, the PHOTAC may be isomerized one or more additional time(s).

In various examples, a method of the present disclosure comprises contacting a cell with a PHOTAC of the present disclosure and/or administering to a subject in need of treatment a composition of the present disclosure with a PHOTAC is in an activated state and then the PHOTAC is exposed to electromagnetic radiation to isomerize the PHOTAC to deactivated state. The PHOTAC may be isomerized one or more additional time(s). Alternatively, in various other examples, the method of the present disclosure comprises contacting a cell with a PHOTAC of the present disclosure and/or administering to a subject in need of treatment a composition of the present disclosure with a PHOTAC in a deactivated state and then the PHOTAC is exposed to electromagnetic radiation to isomerize the PHOTAC to an activated state. As before, the PHOTAC may be isomerized one or more additional time(s). Depending on the type of photoswitchable group used, an activated PHOTAC may relax to a deactivated state or a deactivated PHOTAC may relax to an activated state.

In various examples, a method of the present disclosure further comprises exposing the PHOTAC to electromagnetic radiation at a wavelength necessary induce a conformational change (e.g., from relaxed to unstable, unstable to relaxed, cis to trans, trans to cis, activated to deactivated, or deactivated to activated) one or more additional time(s) after an initial exposure to electromagnetic radiation or after a period of time during which relaxation occurs.

In various examples, a PHOTAC of the present disclosure is used in a therapeutically effective amount (e.g., administered to a subject in need of treatment). The term “therapeutically effective amount” as used herein refers to an amount of an agent sufficient to achieve, in a single or multiple doses, the intended purpose of treatment. Treatment does not have to lead to complete cure, although it may. Treatment may mean alleviation of one or more of the symptom(s) and/or marker(s) of the indication. The exact amount desired or required will likely vary depending on the particular compound or composition used, its mode of administration, patient specifics, and the like. An appropriate effective amount may be determined by one of ordinary skill in the art informed by the instant disclosure using only routine experimentation. Treatment may be orientated symptomatically, for example, to suppress symptoms. Treatment can be effected over a short period, over a medium term, or can be a long-term treatment, such as, for example, within the context of a maintenance therapy. Treatment can be continuous or intermittent.

A dose of a therapeutically effective amount of a PHOTAC of the present disclosure may have a concentration of 1 pM to 10 mM (e.g., 1 pM, 1 nM, 1 μM, or the like), including all 0.1 pM values and ranges therebetween. In various examples, a dose of a therapeutically effective amount of a PHOTAC of the present disclosure may have a concentration of 1-500 μM, 50-500 μM, 1-250 μM, 10-250 μM, 25-250 μM, 25-150 μM, 50-250 μM, or 50-150 μM. In various examples, a dose of a therapeutically effective amount of a PHOTAC of the present disclosure may have a concentration of 1-50 nM, 1-100 nM, 1-25 nM, 50 nM to 1 μM, 50-500 nM, 10 nM to 1 μM, 1 nM to 1 μM, 100 nM to 1 μM, 500 nM to 1 μM, 750 nM to 1 μM, 50 nM to 10 μM, 10 nM to 10 μM, 1 nM to 10 μM, 100 nM to 10 μM, 500 nM to 10 μM, 750 nM to 10 μM. In various examples, a dose of a therapeutically effective amount of a PHOTAC of the present disclosure may have a concentration of 1-50 pM, 1-100 pM, 1-25 pM, 50 pM to 1 μM, 50-500 pM, 10 pM to 1 μM, 1 pM to 1 μM, 100 pM to 1 μM, 500 pM to 1 μM, 750 pM to 1 μM, 50 pM to 10 μM, 10 pM to 10 μM, 1 pM to 10 μM, 100 pM to 10 μM, 500 pM to 10 μM, 750 pM to 10 μM. In various examples, a dose of a therapeutically effective amount of a PHOTAC of the present disclosure may have a concentration of 50 pM to 1 nM, 10 pM to 1 nM, 1 pM to 1 nM, 100 pM to 1 nM, 500 pM to 1 nM, 750 pM to 1 nM, 50 pM to 10 nM, 10 pM to 10 nM, 1 pM to 10 nM, 100 pM to 10 nM, 500 pM to 10 nM, 750 pM to 10 nM.

The steps of the method described in the various embodiments and examples disclosed herein are sufficient to carry out the methods of the present disclosure. Thus, in an example, a method consists essentially of a combination of the steps of the methods disclosed herein. In another example, a method consists of such steps.

The following Statements describe various non-limiting examples compounds (e.g., PHOTACs) and methods of the present disclosure.

Statement 1. A compound (e.g., PHOTAC) having the following structure: A-PS-L-B, A-L-PS-B, PS-A-L-B, PS-A-L-B-PS, A-PS-L-PS-B, A-L-PS-L-B, PS-A-L-PS-B, A-PS-L-B-PS, or A′-L′-B′, where A is an E3 ligase ligand, PS is a photoswitchable group, L is optional and is a linker, B is a ligand for a target protein, A′ is an E3 ligase ligand optionally comprising a photoswitchable group, L′ is an optional linker optionally comprising a photoswitchable group, and B′ is a ligand for a target protein optionally comprising a photoswitchable group.
Statement 2. A compound according to Statement 1, where the photoswitchable group is chosen from:

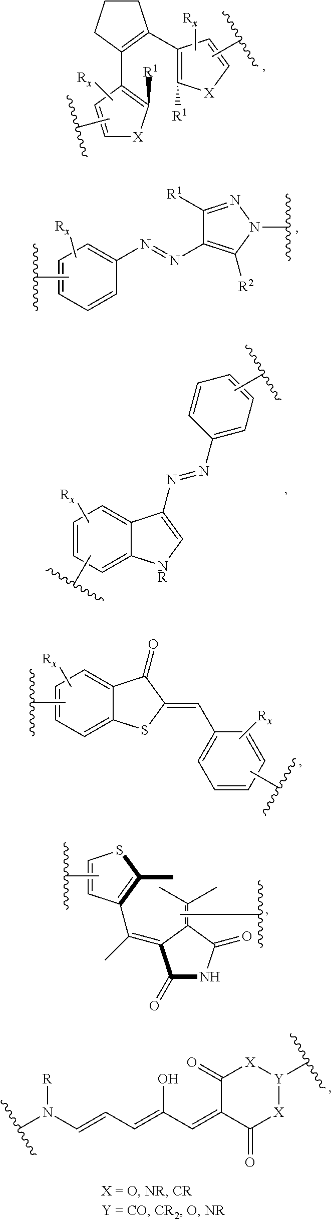

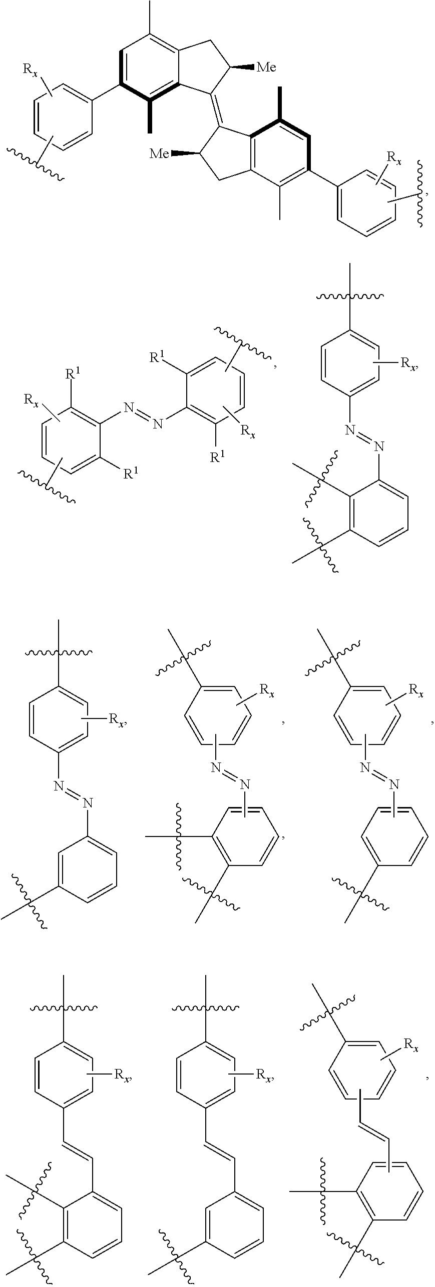

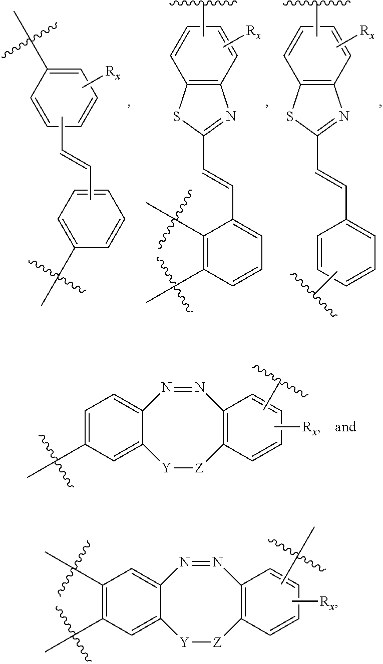

where R¹ is F, Cl, Me, or OMe; R is H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂; x is 0, 1, 2, 3, 4, or 5; X is methylene, C═O, or C═S, and Y is methylene, O, or S; and Z is methylene, O, or S.
Statement 3. A compound according to Statement 1 or Statement 2, where the linker is chosen from:

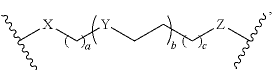

where Y is methylene or O, X and Z are independently methylene, O, NH, S,

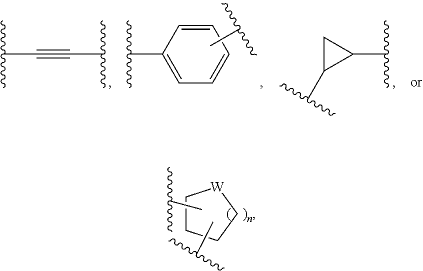

where W is methylene, NH, O, or S and n is greater than or equal to 0 (e.g., 0, 1, 2, 3, or 4); and a is 0-10, including every integer value and range therebetween, b is 0-10, including every integer value and range therebetween, and c is 0-10, including every integer value and range therebetween;

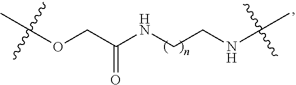

where n is 2, 3, 4, or 5;
groups formed from polyethylene glycol groups (e.g.,

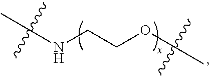

where x is 1-4;

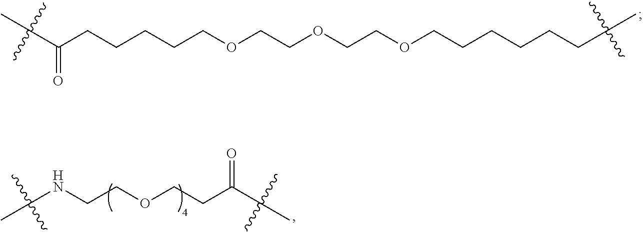

and the like); alkyl linkers (e.g.,

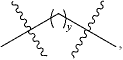

where y is 1-20;

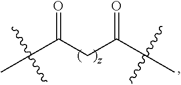

where z is 1-20; and the like); and peptide-based linkers (e.g., -SGSG- and the like).
Statement 4. A compound according to any one of the preceding Statements, where the E3 ligase ligand is a ligand for VHL, CRBN, RNF114, MDM2, DCAF15, DCAF16, Keap1, and/or SCF.
Statement 5. A compound according to any one of the preceding Statements, where E3 ligase ligand is chosen from:

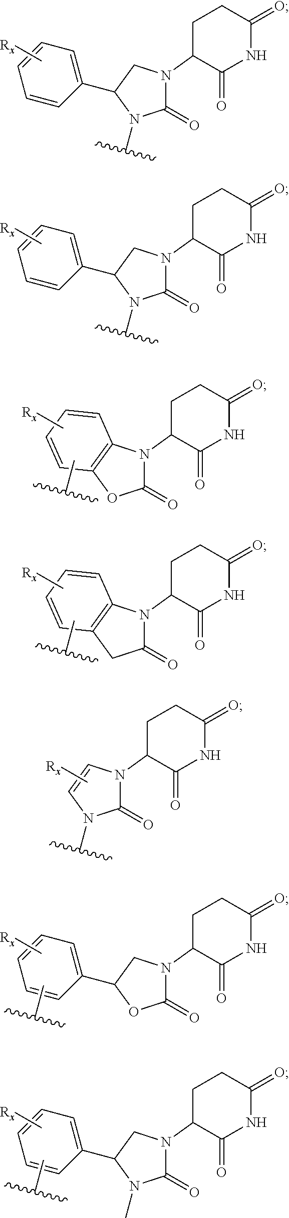

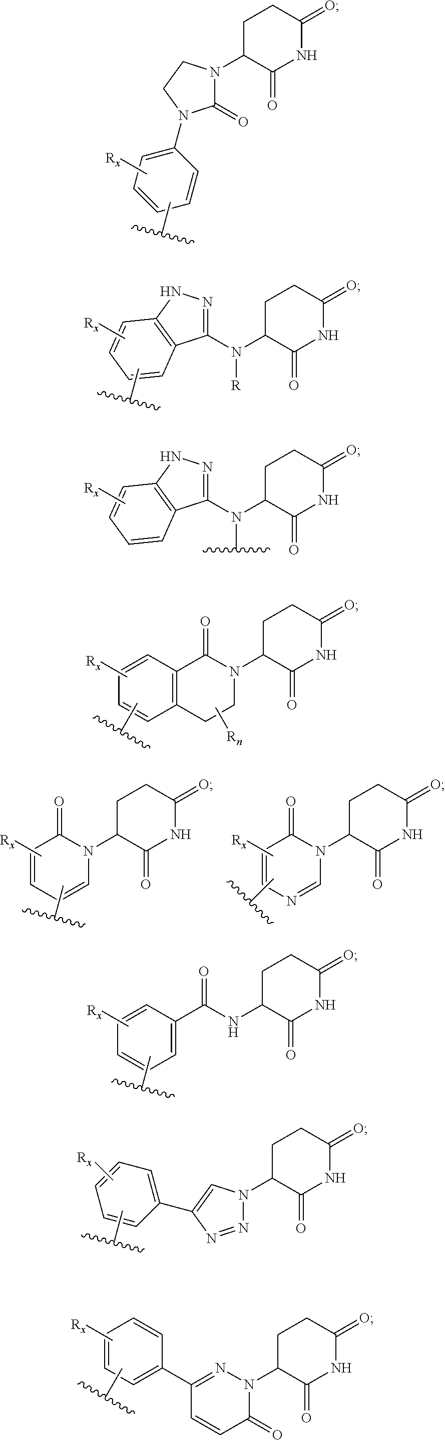

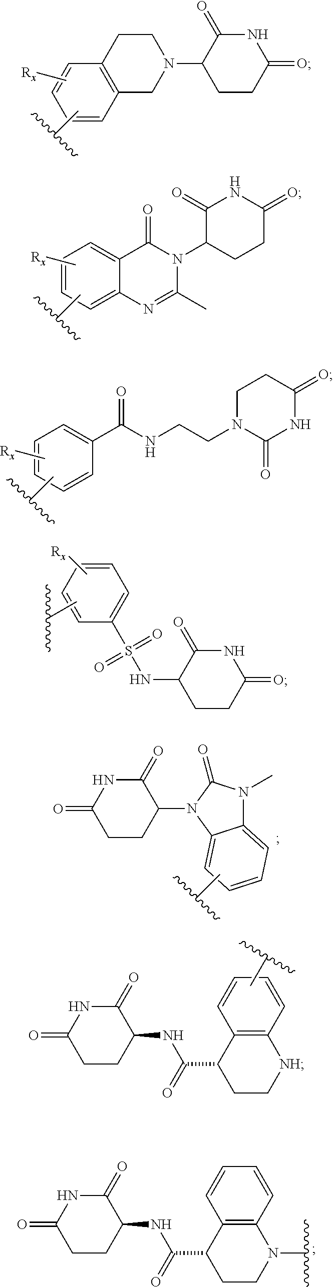

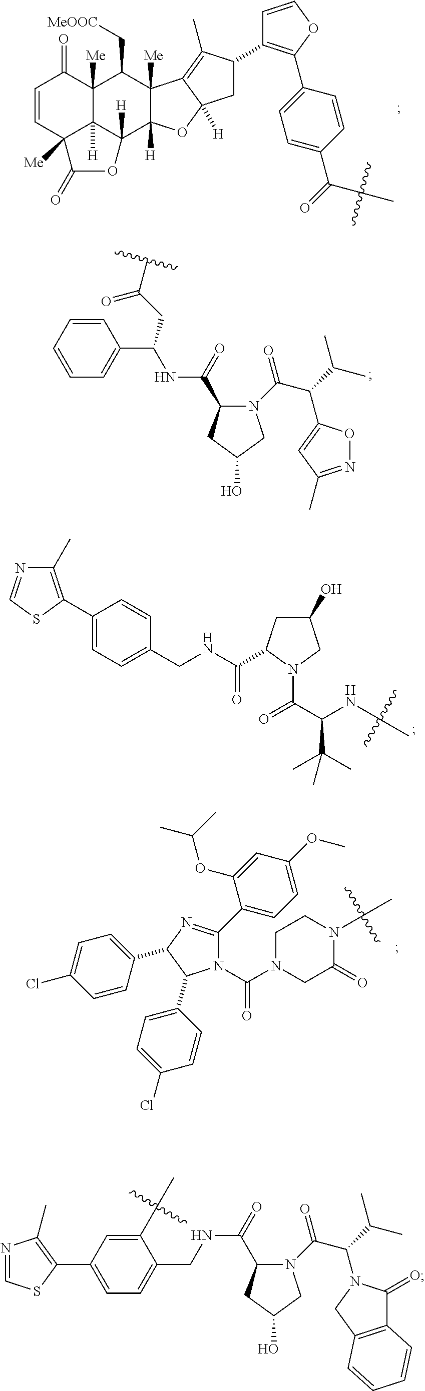

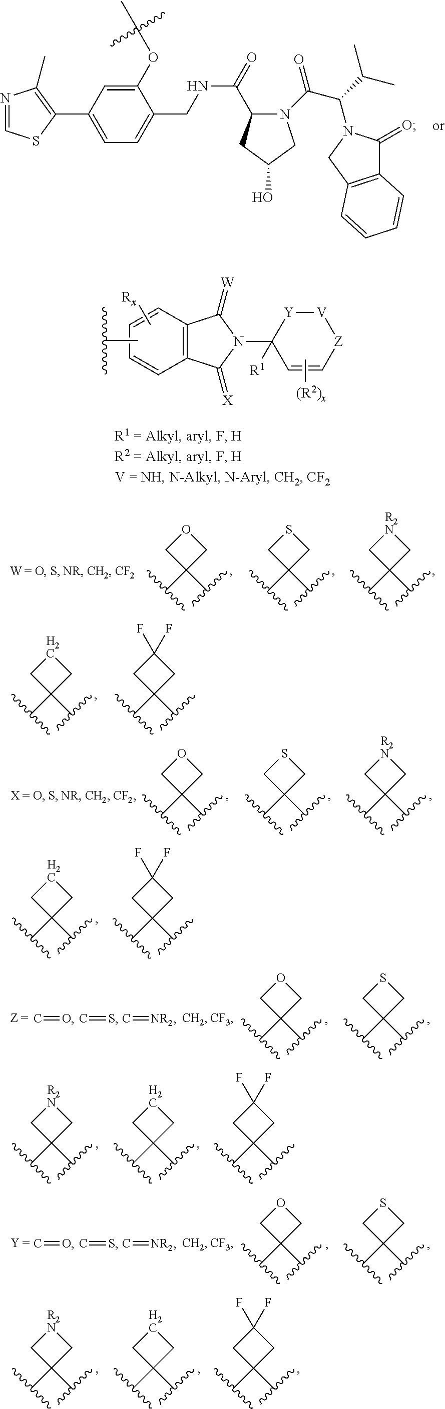

where R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, a photoswitchable group, H2N-(D-R)₈-PIYALA- (SEQ ID NO:1), GGGGGGRAEDS*GNES*EGE-COOH (SEQ ID NO:2), where * is a phosphorylated serine, and GGGGGGDRIIDS*GLDS*M-COOH (SEQ ID NO:3), where * is a phosphorylated serine, x is 0, 1, 2, 3, 4, or 5.
Statement 6. A compound according to any one of the preceding Statements, where the ligand for a target protein is chosen from:

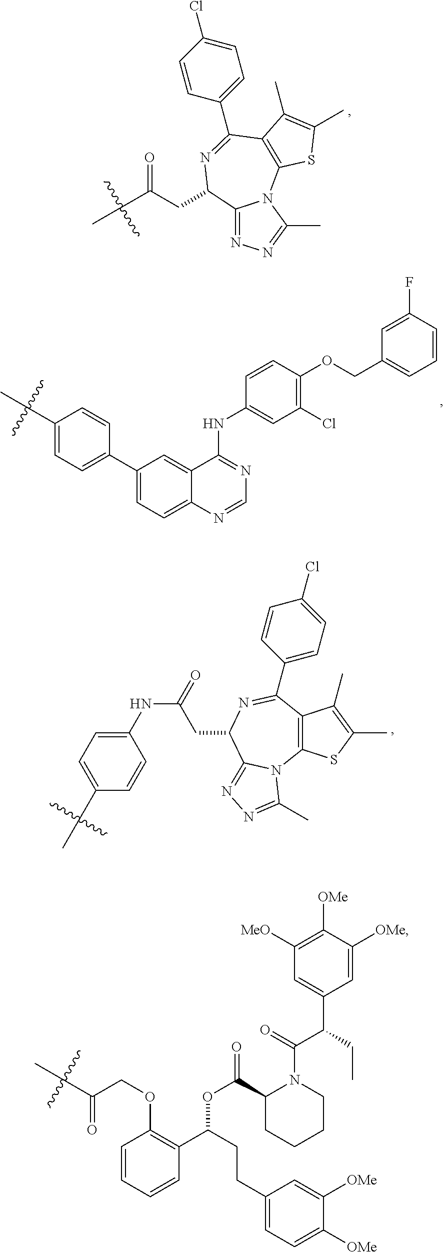

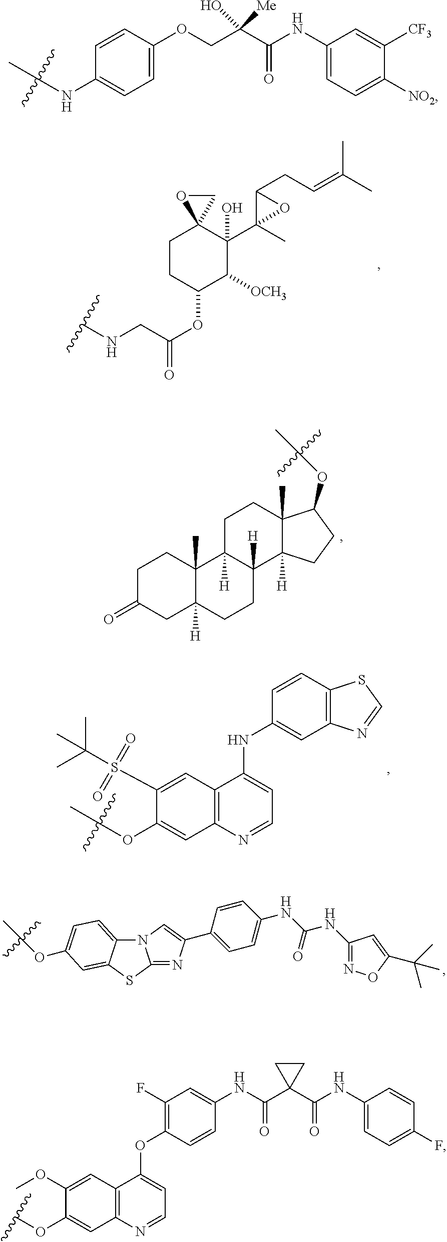

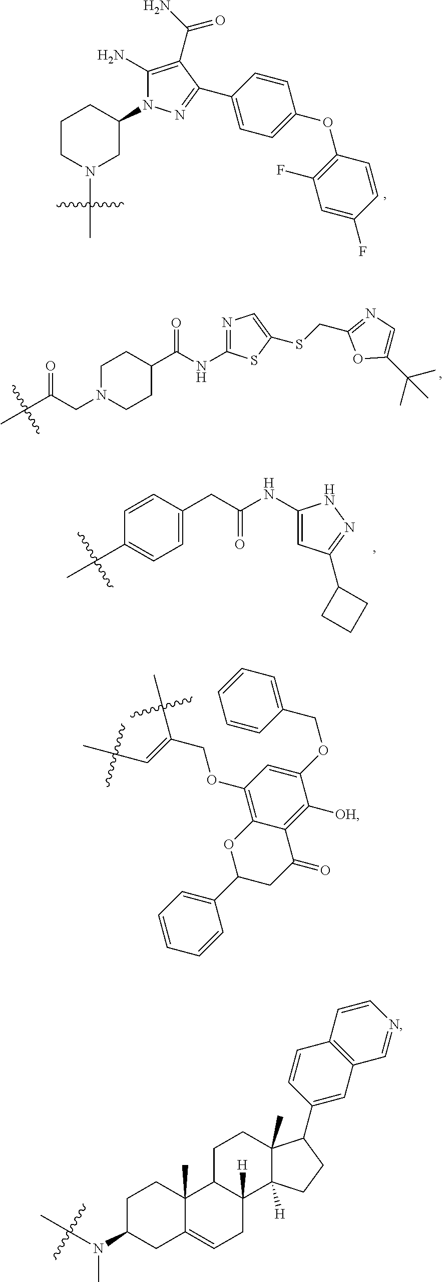

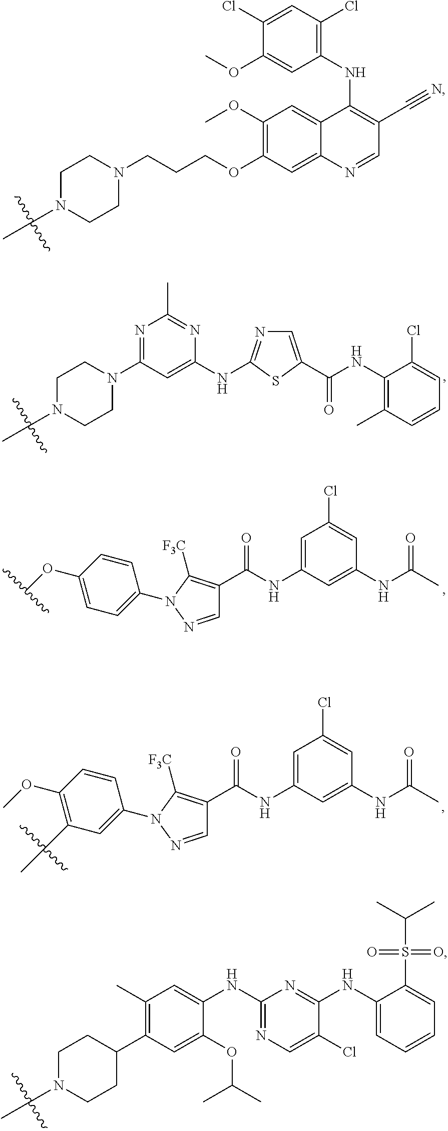

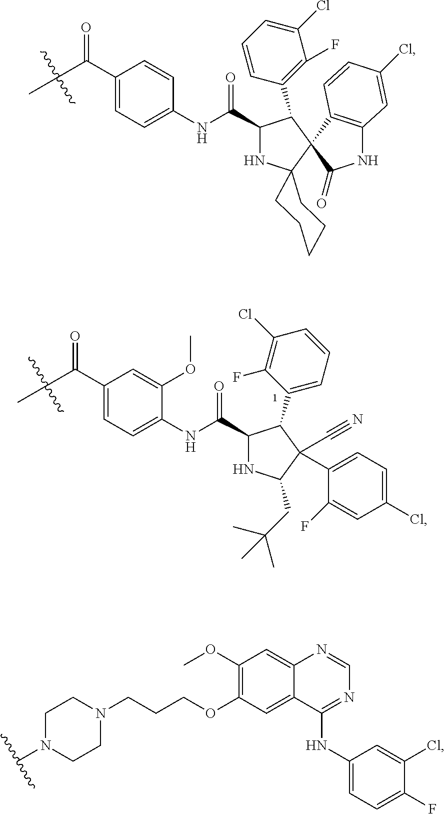 [SMILES: *C(=O)c1ccc(NC(=O)[C@@H]2NC3(CCCCC3)[C@]3(C(=O)Nc4cc(Cl)ccc43)[C@H]2c2cccc(Cl)c2F)cc1 ; *C(=O)c1ccc(NC(=O)[C@@H]2N[C@@H](CC(C)(C)C)C(C#N)(c3ccc(Cl)cc3F)[C@H]2c2cccc(Cl)c2F)c(OC)c1 ; *N1CCN(CCCOc2cc3c(Nc4ccc(F)c(Cl)c4)ncnc3cc2OC)CC1]

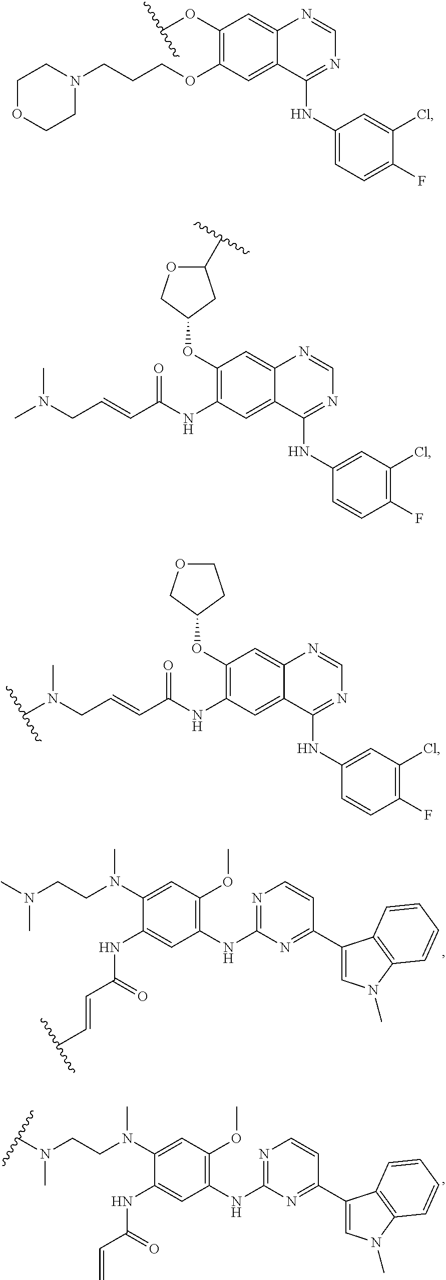 [SMILES: *Oc1cc2ncnc(Nc3ccc(F)c(Cl)c3)c2cc1OCCCN1CCOCC1 ; *C1C[C@H](Oc2cc3ncnc(Nc4ccc(F)c(Cl)c4)c3cc2NC(=O)/C=C/CN(C)C)CO1 ; *N(C)C/C=C/C(=O)Nc1cc2c(Nc3ccc(F)c(Cl)c3)ncnc2cc1O[C@H]1CCOC1 ; */C=C/C(=O)Nc1cc(Nc2nccc(-c3cn(C)c4ccccc34)n2)c(OC)cc1N(C)CCN(C)C ; *N(C)CCN(C)c1cc(OC)c(Nc2nccc(-c3cn(C)c4ccccc34)n2)cc1NC(=O)C=C]

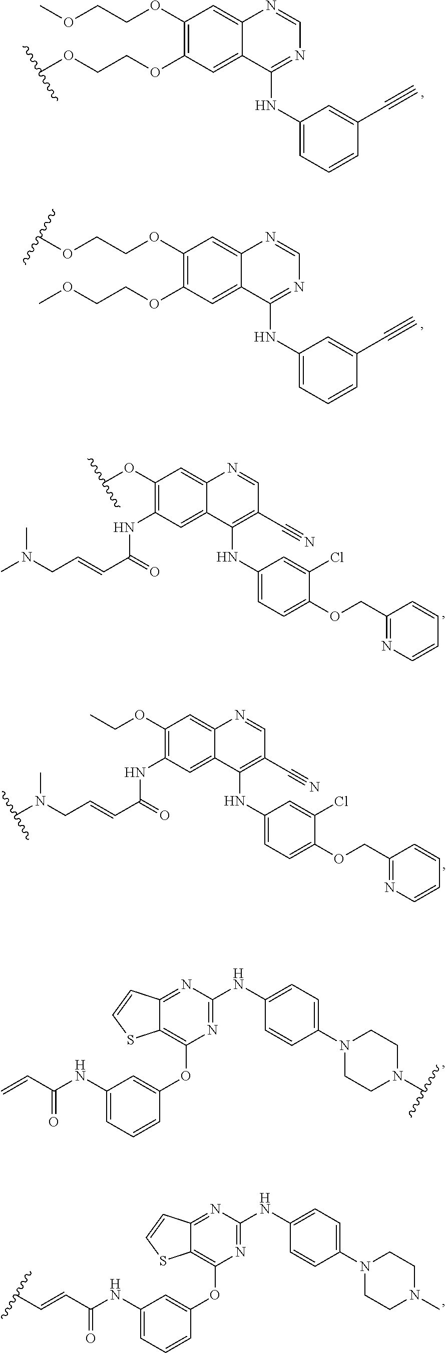

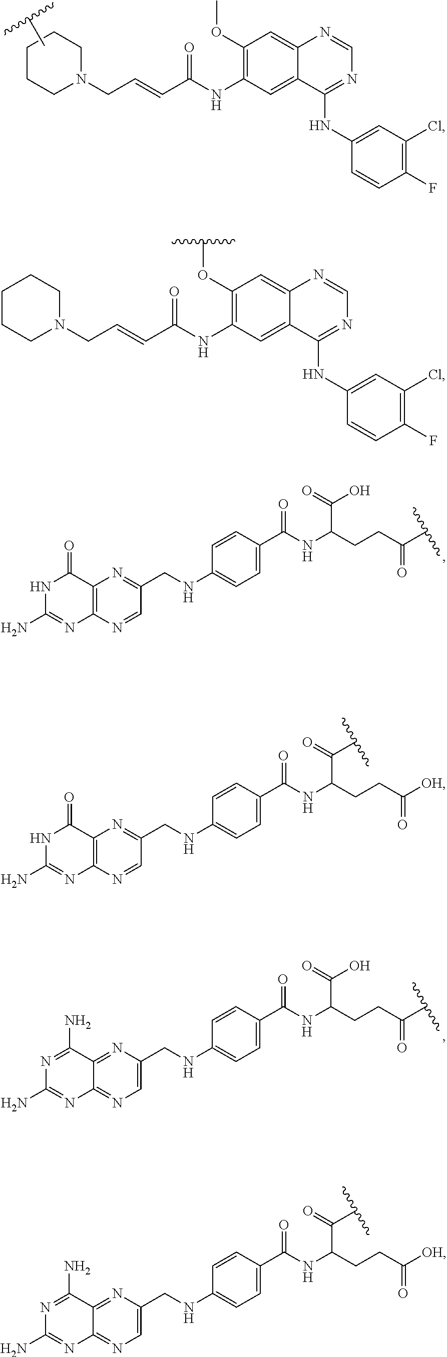

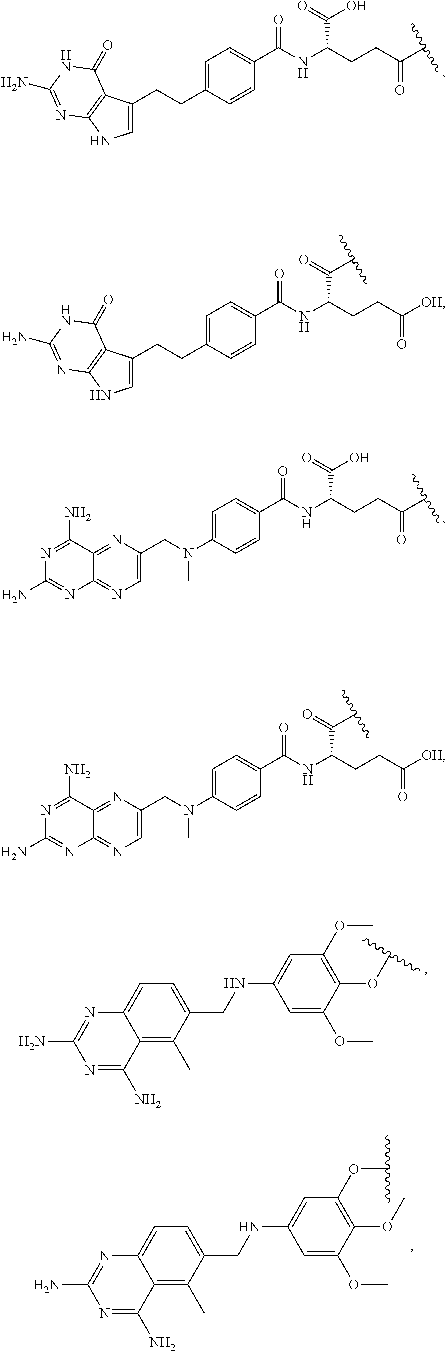 [SMILES: *C(=O)CC[C@H](NC(=O)c1ccc(CCc2c[nH]c3nc(N)[nH]c(=O)c23)cc1)C(=O)O ; *C(=O)[C@H](CCC(=O)O)NC(=O)c1ccc(CCc2c[nH]c3nc(N)[nH]c(=O)c23)cc1 ; *C(=O)CC[C@H](NC(=O)c1ccc(N(C)Cc2cnc3nc(N)nc(N)c3n2)cc1)C(=O)O ; *C(=O)[C@H](CCC(=O)O)NC(=O)c1ccc(N(C)Cc2cnc3nc(N)nc(N)c3n2)cc1 ; *Oc1c(OC)cc(NCc2ccc3nc(N)nc(N)c3c2C)cc1OC ; *Oc1cc(NCc2ccc3nc(N)nc(N)c3c2C)cc(OC)c1OC]

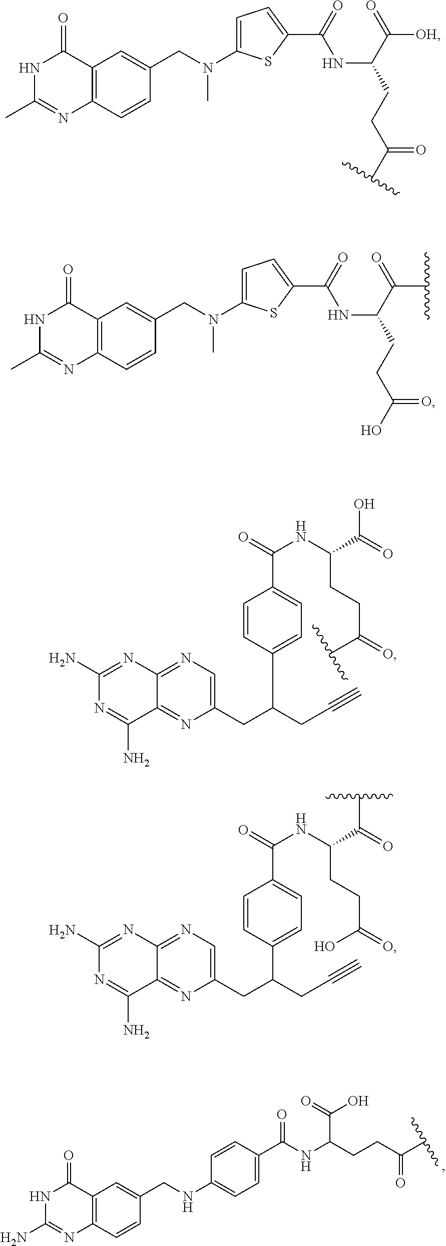 [SMILES: *C(=O)CC[C@H](NC(=O)c1ccc(N(C)Cc2ccc3nc(C)[nH]c(=O)c3c2)s1)C(=O)O ; *C(=O)[C@H](CCC(=O)O)NC(=O)c1ccc(N(C)Cc2ccc3nc(C)[nH]c(=O)c3c2)s1 ; *C(=O)CC[C@H](NC(=O)c1ccc(C(CC#C)Cc2cnc3nc(N)nc(N)c3n2)cc1)C(=O)O ; *C(=O)[C@H](CCC(=O)O)NC(=O)c1ccc(C(CC#C)Cc2cnc3nc(N)nc(N)c3n2)cc1 ; *C(=O)CCC(NC(=O)c1ccc(NCc2ccc3nc(N)[nH]c(=O)c3c2)cc1)C(=O)O]

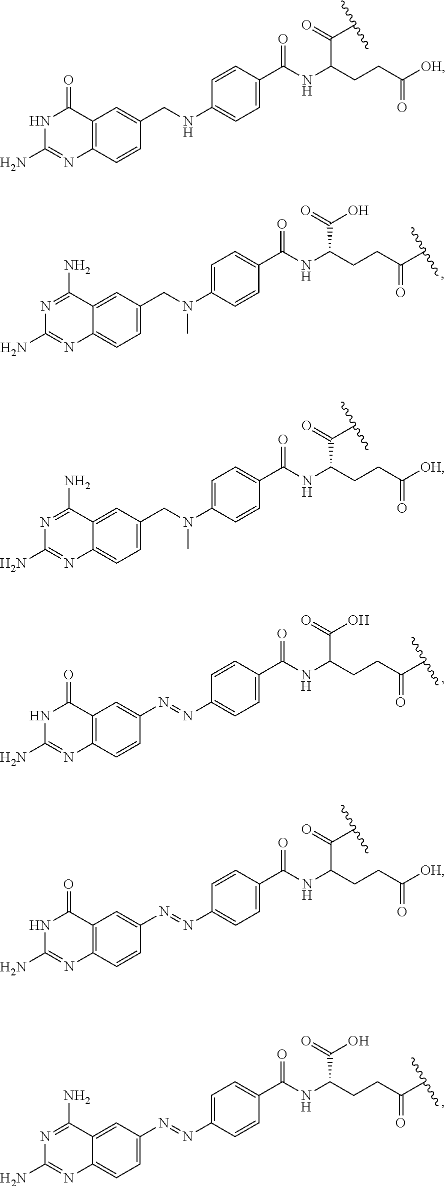 [SMILES: *C(=O)C(CCC(=O)O)NC(=O)c1ccc(NCc2ccc3nc(N)[nH]c(=O)c3c2)cc1 ; *C(=O)CC[C@H](NC(=O)c1ccc(N(C)Cc2ccc3nc(N)nc(N)c3c2)cc1)C(=O)O ; *C(=O)[C@H](CCC(=O)O)NC(=O)c1ccc(N(C)Cc2ccc3nc(N)nc(N)c3c2)cc1 ; *C(=O)CCC(NC(=O)c1ccc(/N=N/c2ccc3nc(N)[nH]c(=O)c3c2)cc1)C(=O)O ; *C(=O)C(CCC(=O)O)NC(=O)c1ccc(/N=N/c2ccc3nc(N)[nH]c(=O)c3c2)cc1 ; *C(=O)CC[C@H](NC(=O)c1ccc(/N=N/c2ccc3nc(N)nc(N)c3c2)cc1)C(=O)O]

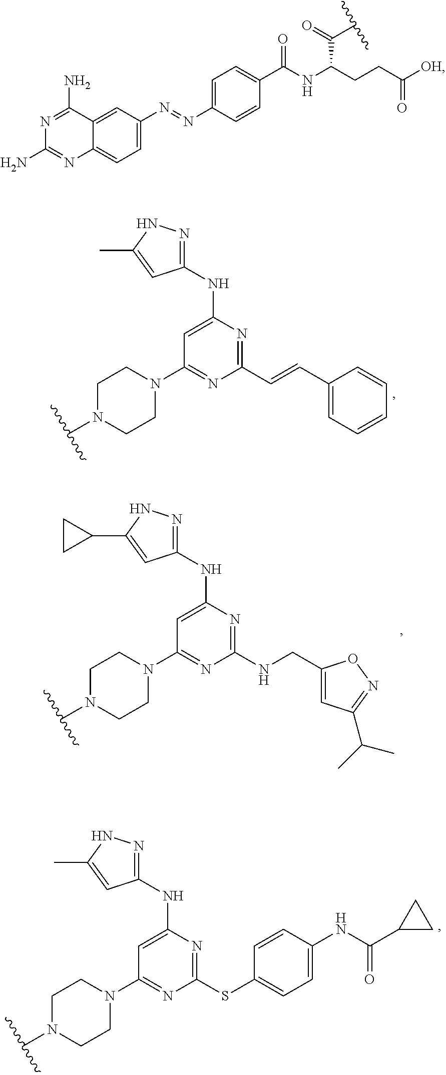 [SMILES: *C(=O)[C@H](CCC(=O)O)NC(=O)c1ccc(/N=N/c2ccc3nc(N)nc(N)c3c2)cc1 ; *N1CCN(c2cc(Nc3cc(C)[nH]n3)nc(/C=C/c3ccccc3)n2)CC1 ; *N1CCN(c2cc(Nc3cc(C4CC4)[nH]n3)nc(NCc3cc(C(C)C)no3)n2)CC1 ; *N1CCN(c2cc(Nc3cc(C)[nH]n3)nc(Sc3ccc(NC(=O)C4CC4)cc3)n2)CC1]

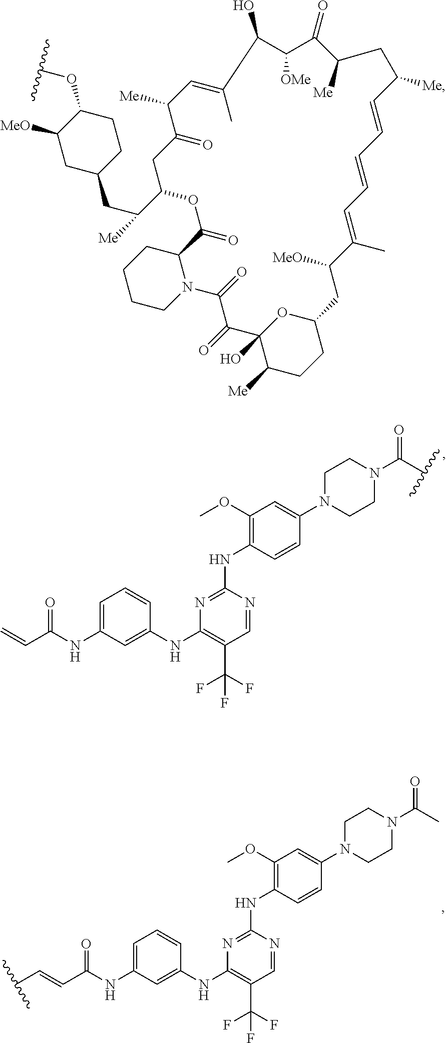 [SMILES: *O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; *C(=O)N1CCN(c2ccc(Nc3ncc(C(F)(F)F)c(Nc4cccc(NC(=O)C=C)c4)n3)c(OC)c2)CC1 ; */C=C/C(=O)Nc1cccc(Nc2nc(Nc3ccc(N4CCN(C(C)=O)CC4)cc3OC)ncc2C(F)(F)F)c1]

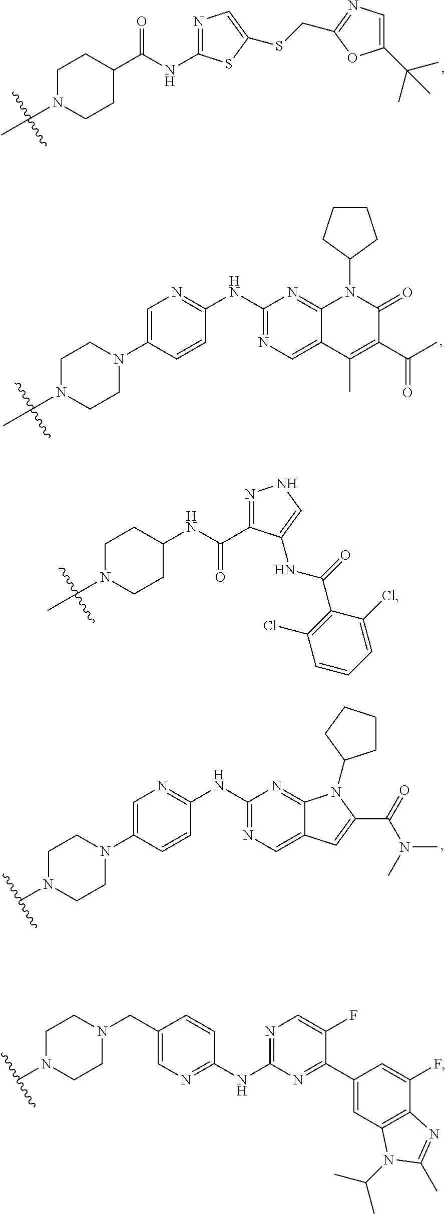

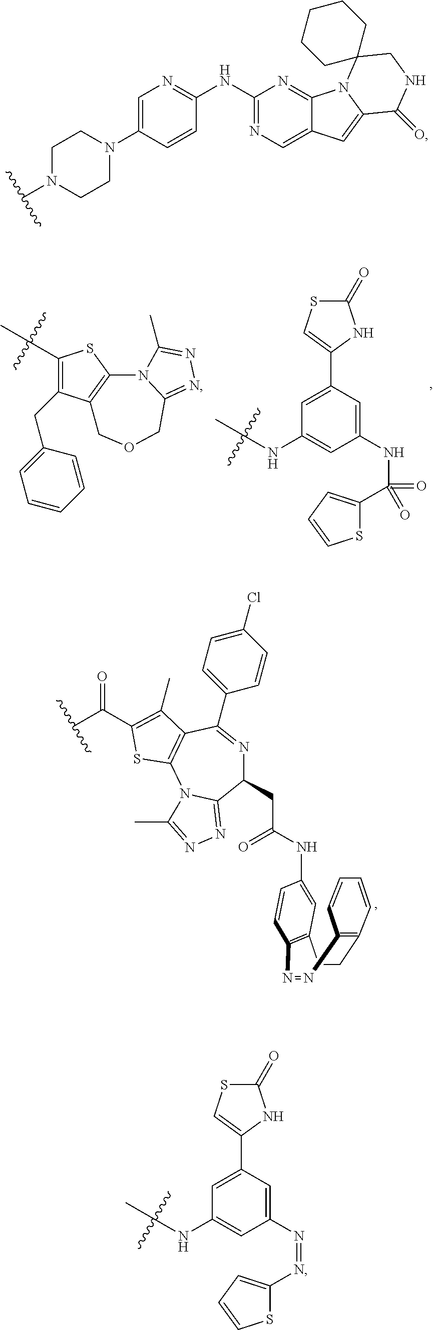

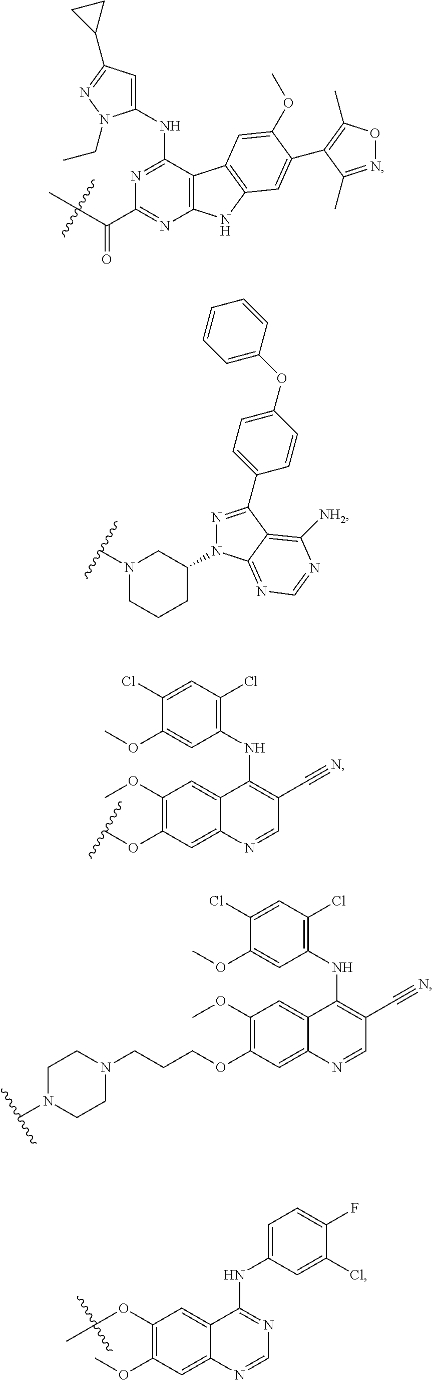 [SMILES: *C(=O)c1nc(Nc2cc(C3CC3)nn2CC)c2c(n1)[nH]c1cc(-c3c(C)noc3C)c(OC)cc12 ; *N1CCC[C@@H](n2nc(-c3ccc(Oc4ccccc4)cc3)c3c(N)ncnc32)C1 ; *Oc1cc2ncc(C#N)c(Nc3cc(OC)c(Cl)cc3Cl)c2cc1OC ; *N1CCN(CCCOc2cc3ncc(C#N)c(Nc4cc(OC)c(Cl)cc4Cl)c3cc2OC)CC1 ; *Oc1cc2c(Nc3ccc(F)c(Cl)c3)ncnc2cc1OC]

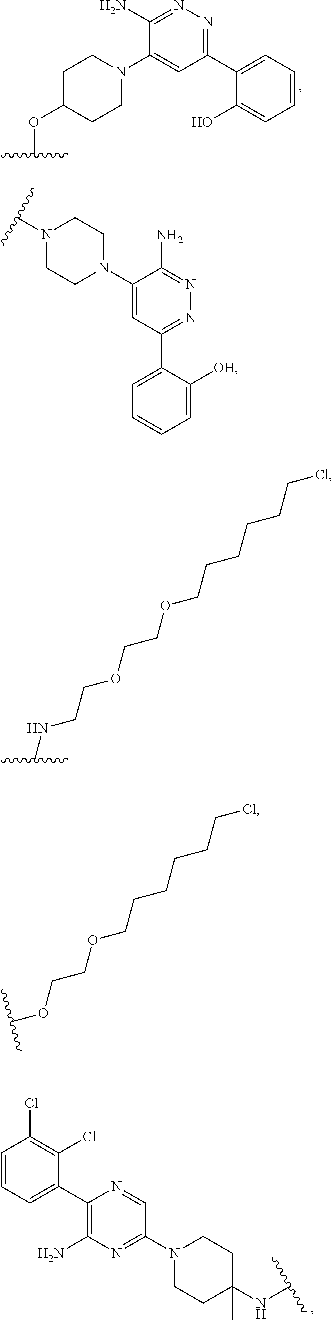 [SMILES: *OC1CCN(c2cc(-c3ccccc3O)nnc2N)CC1 ; *N1CCN(c2cc(-c3ccccc3O)nnc2N)CC1 ; *NCCOCCOCCCCCCCl ; *OCCOCCCCCCCl ; *NC1(C)CCN(c2cnc(-c3cccc(Cl)c3Cl)c(N)n2)CC1]

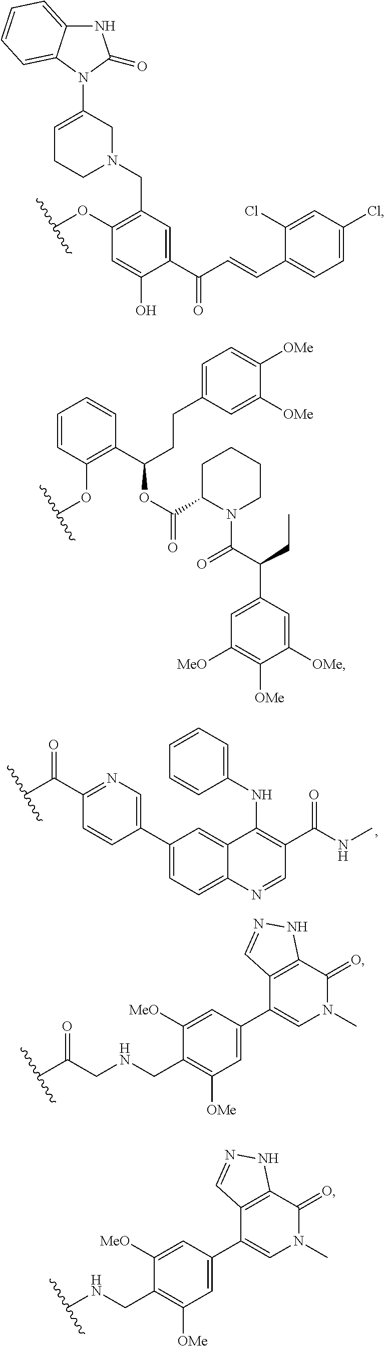

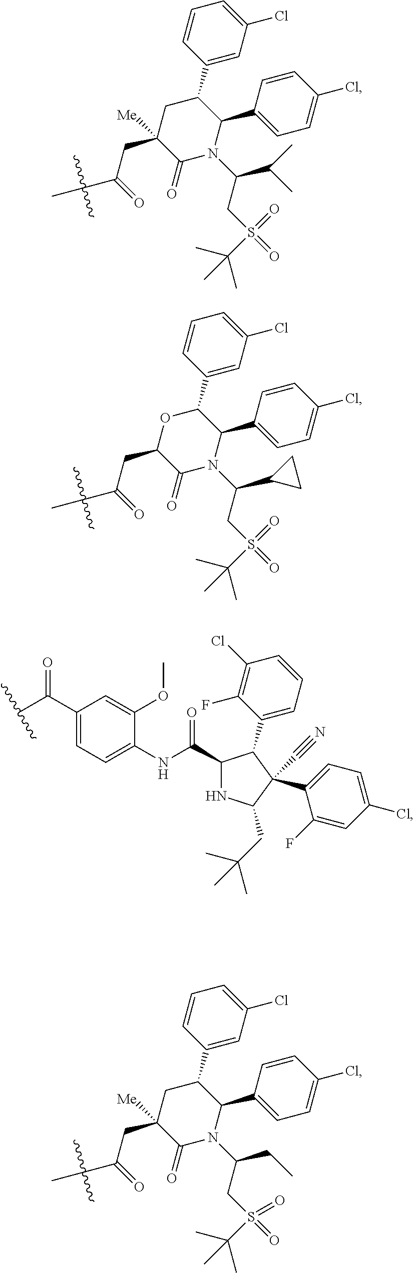

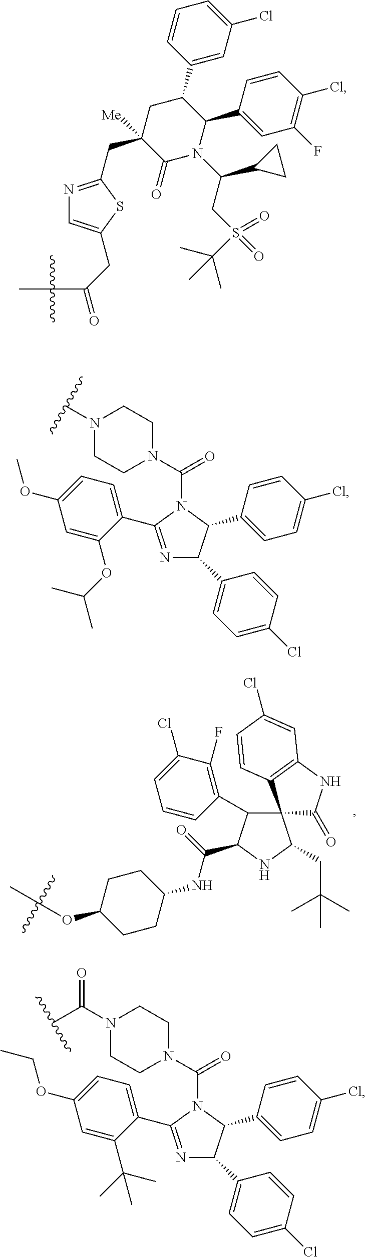

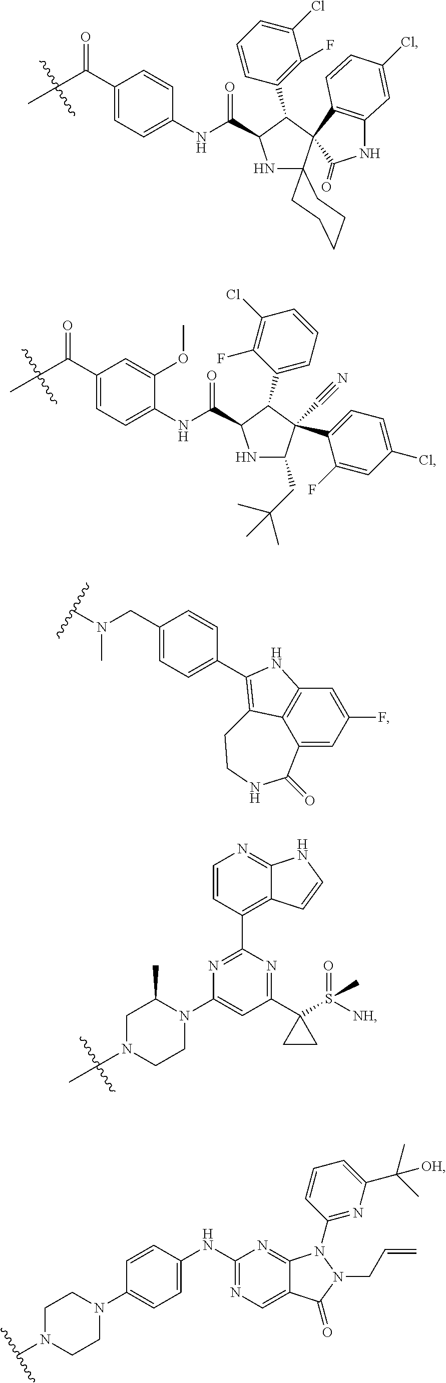

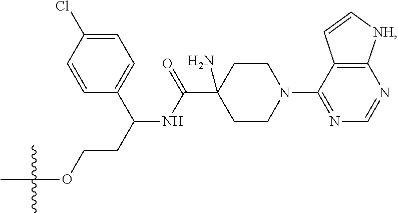 [SMILES: *OCCC(NC(=O)C1(N)CCN(c2ncnc3[nH]ccc23)CC1)c1ccc(Cl)cc1]

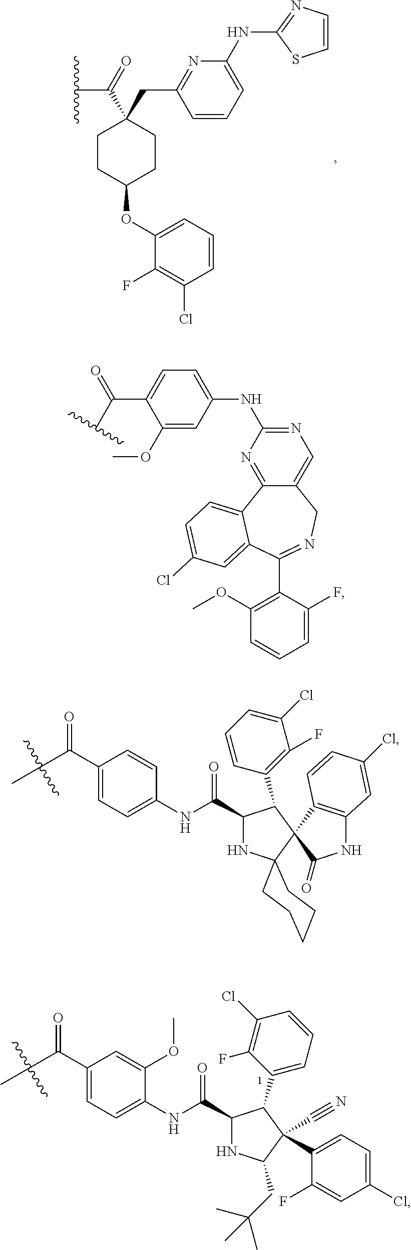

### and GQEDATADDQYQQY (SEQ ID NO:4).

Statement 7. A compound according to any one of the preceding Statements, where the compound has the following structure:

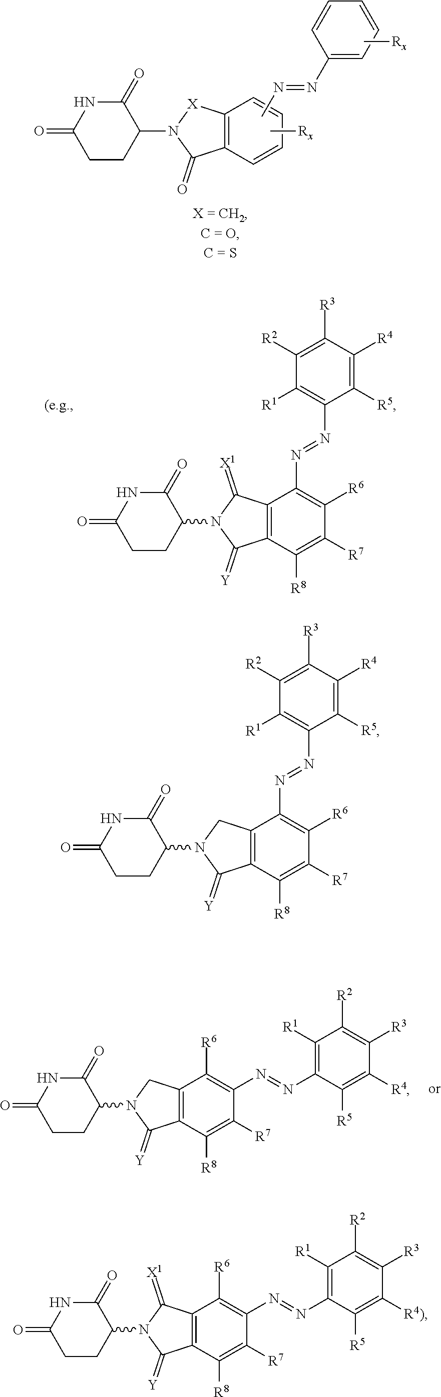

where Y and X¹ are independently chosen from O and S; R¹-R⁷ are independently chosen from H, halogen (e.g., F, Cl, and the like), alkyl (e.g., Me and the like), S-alkyl, NH-alkyl, cycloalkyl, aryl, heteroaryl, heterocyclyl, OH, O-alkyl (e.g., OMe and the like), O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂, and where R¹-R⁵ is also chosen from L-B*, where L is optional and is a linker and B* is a ligand for a target protein, and x is 0, 1, 2, 3, 4, or 5.
Statement 8. A compound according to any one of the preceding Statements, where the compound has the following structure:

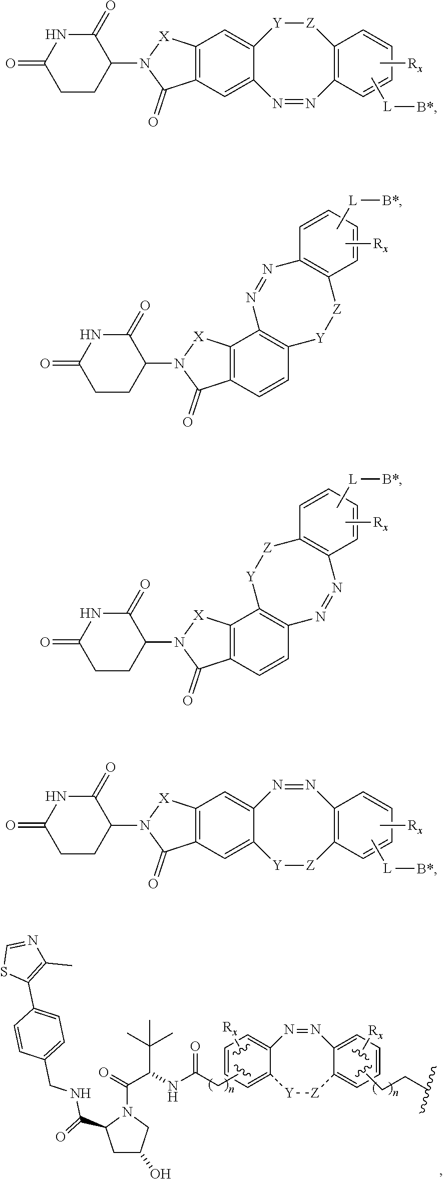

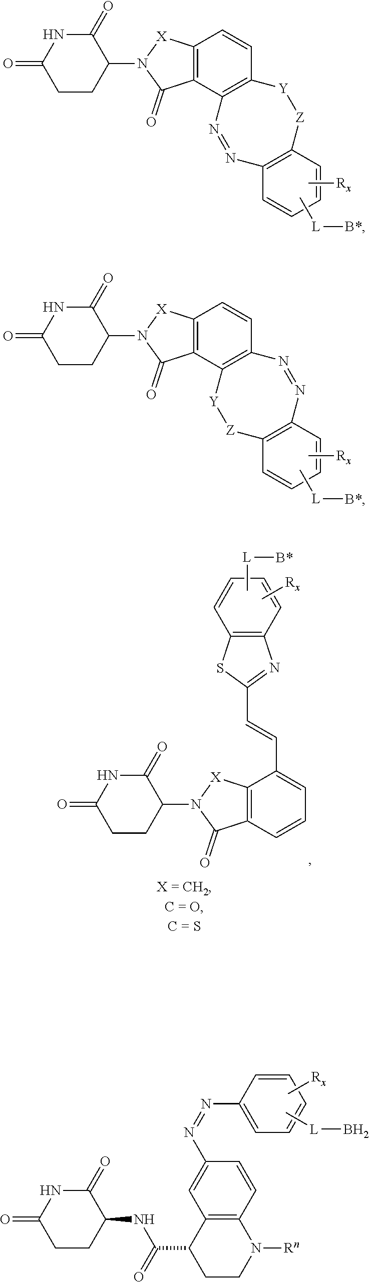

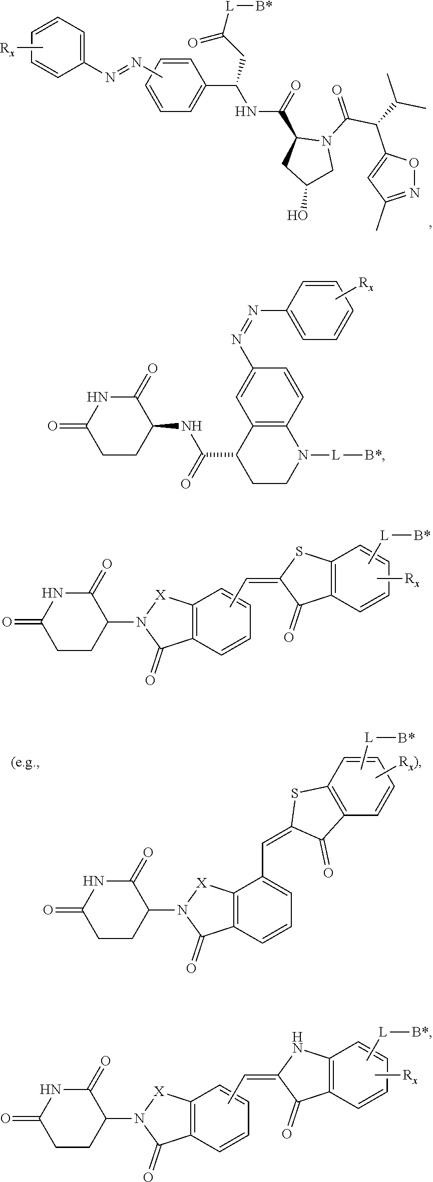

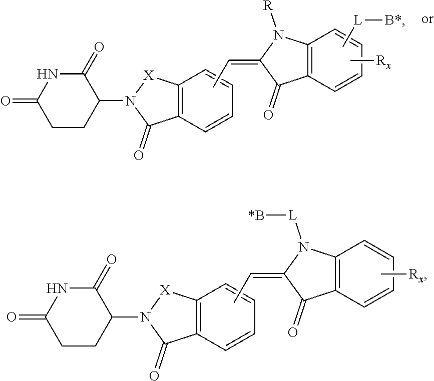

where X is methylene, C═O, or C═S; Y and Z are independently methylene, O, or S; R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, and L-B*, where L is optional and is a linker and B* is a ligand for a target protein, and x is 0, 1, 2, 3, 4, or 5.
Statement 9. A compound of any one of the preceding Statements, where the compound has the following structure:

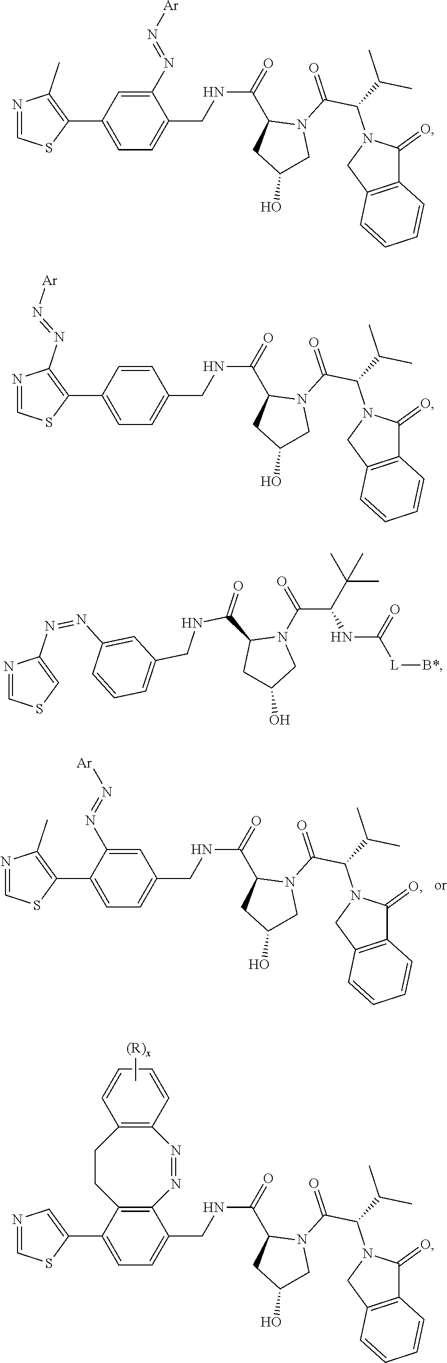

where x is, in various examples, 0, 1, 2, 3, or 4 and R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH— alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, and L-B*, where L is optional and is a linker and B* is a ligand for a target protein; Ar is an aryl group

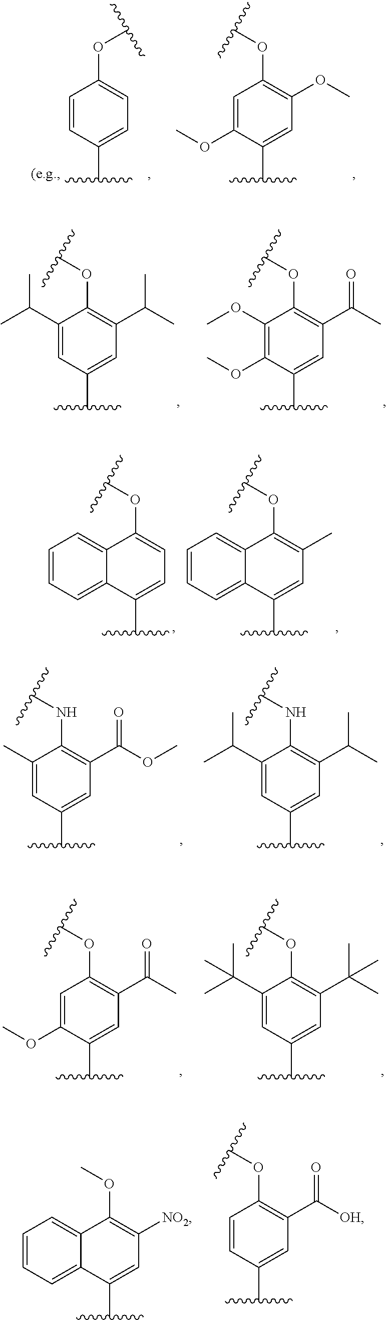

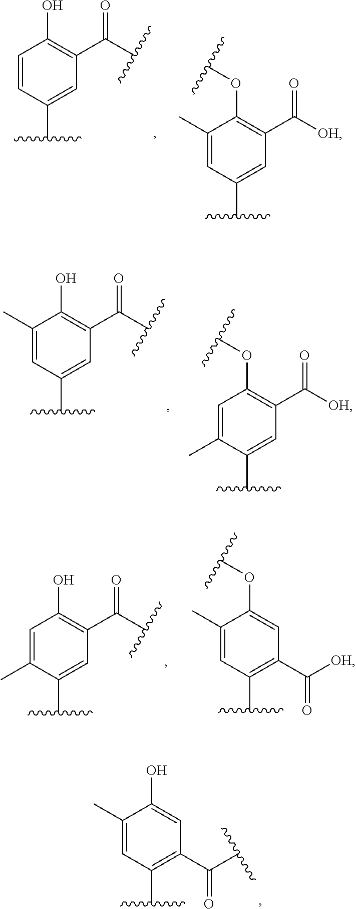

and the like) that is further attached to a linker that is attached to a ligand for a target protein (e.g., protein of interest), or to a ligand of interest.
Statement 10. A compound of any one of the preceding Statements, where the compound has the following structure:

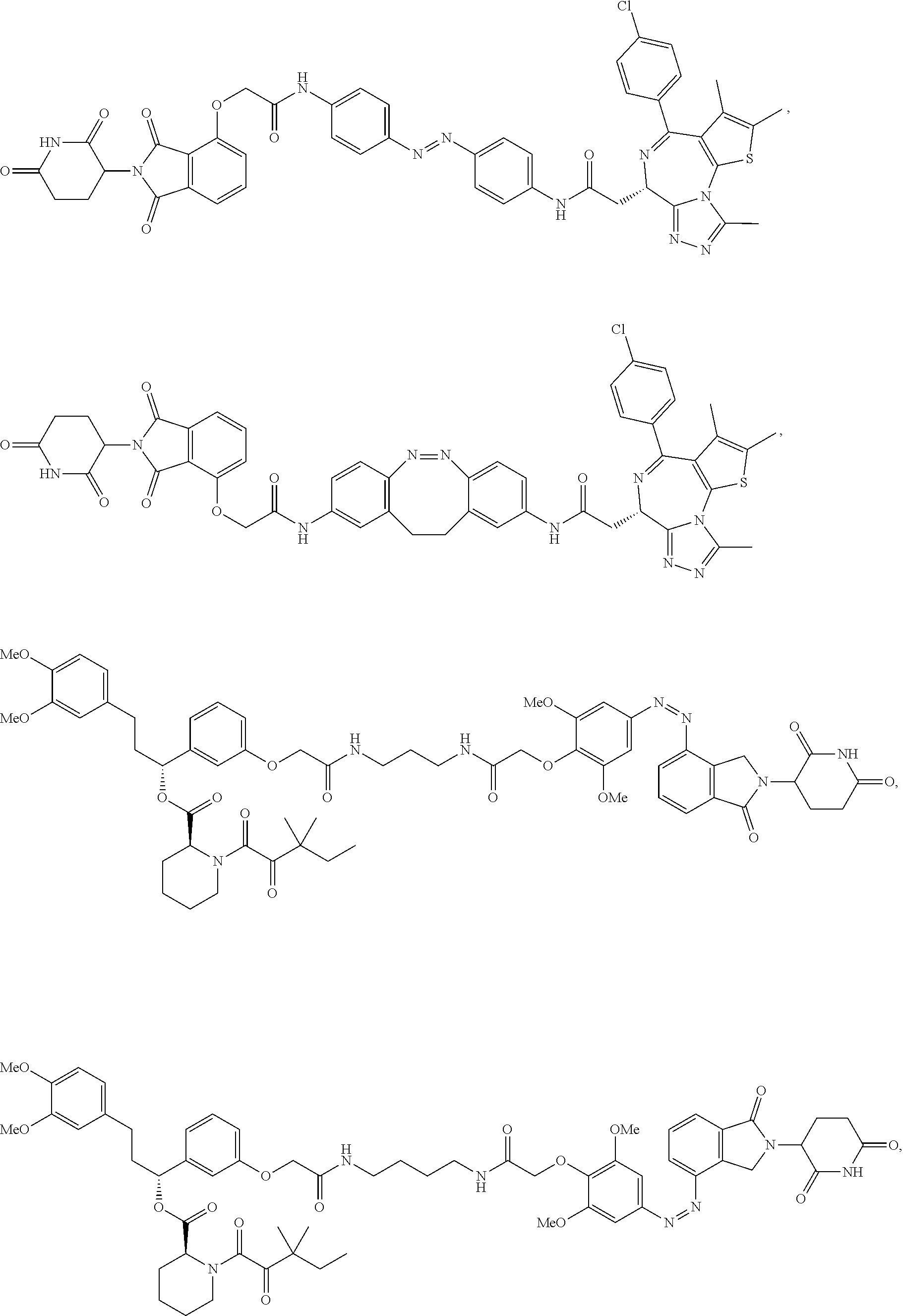

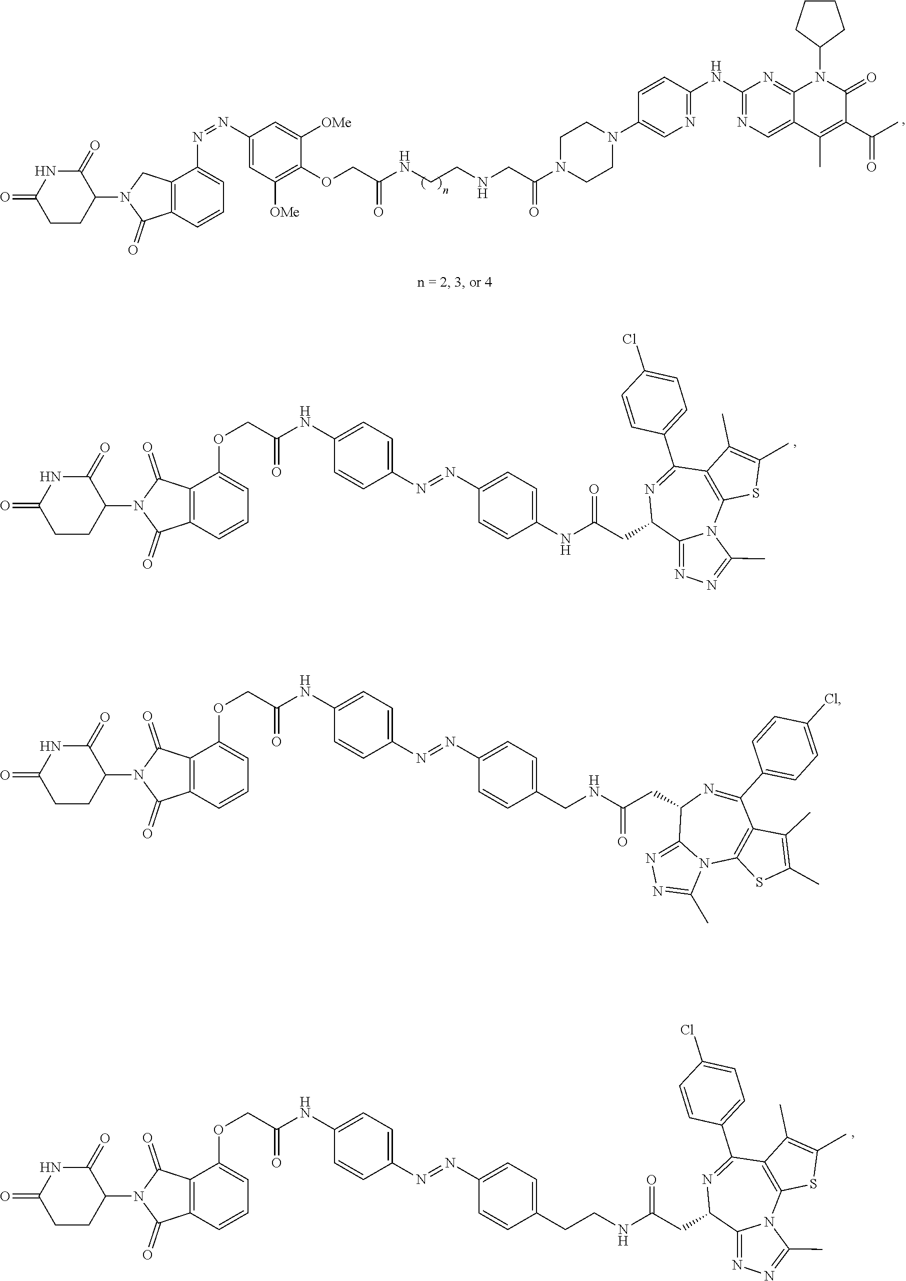

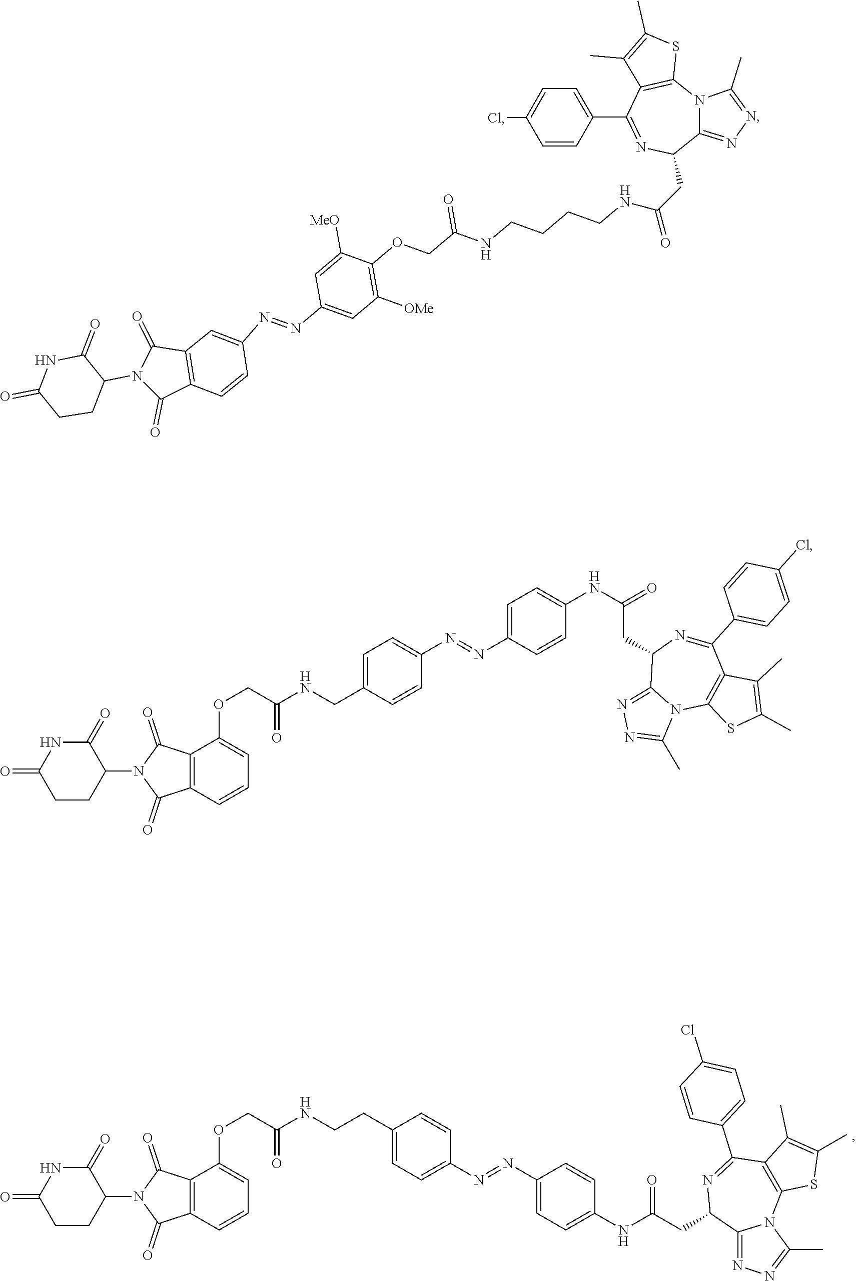 [SMILES: COc1cc(/N=N/c2ccc3c(c2)C(=O)N(C2CCC(=O)NC2=O)C3=O)cc(OC)c1OCC(=O)NCCCCNC(=O)C[C@@H]1N=C(c2ccc(Cl)cc2)c2c(sc(C)c2C)-n2c(C)nnc21 ; Cc1sc2c(c1C)C(c1ccc(Cl)cc1)=N[C@@H](CC(=O)Nc1ccc(/N=N/c3ccc(CNC(=O)COc4cccc5c4C(=O)N(C4CCC(=O)NC4=O)C5=O)cc3)cc1)c1nnc(C)n1-2 ; Cc1sc2c(c1C)C(c1ccc(Cl)cc1)=N[C@@H](CC(=O)Nc1ccc(/N=N/c3ccc(CCNC(=O)COc4cccc5c4C(=O)N(C4CCC(=O)NC4=O)C5=O)cc3)cc1)c1nnc(C)n1-2]

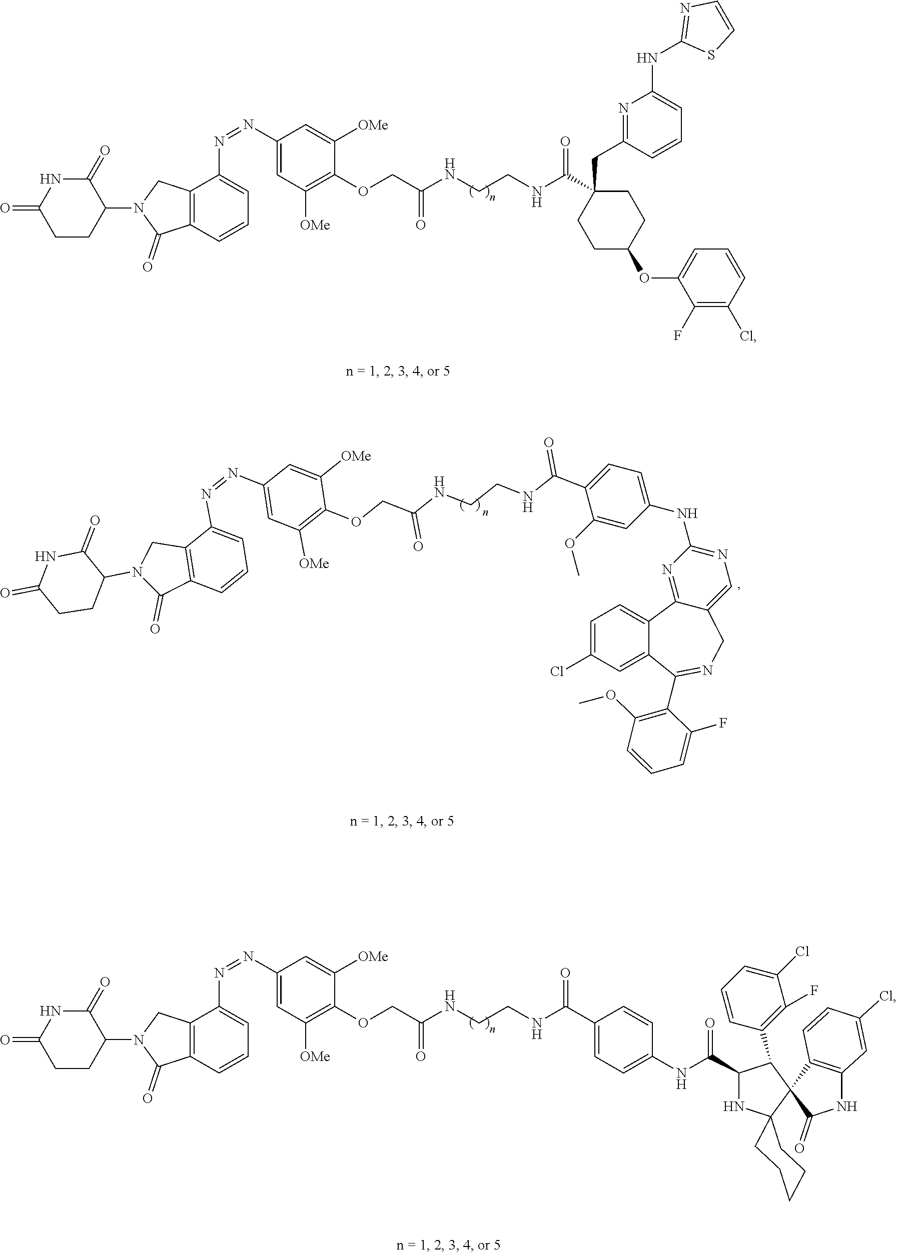

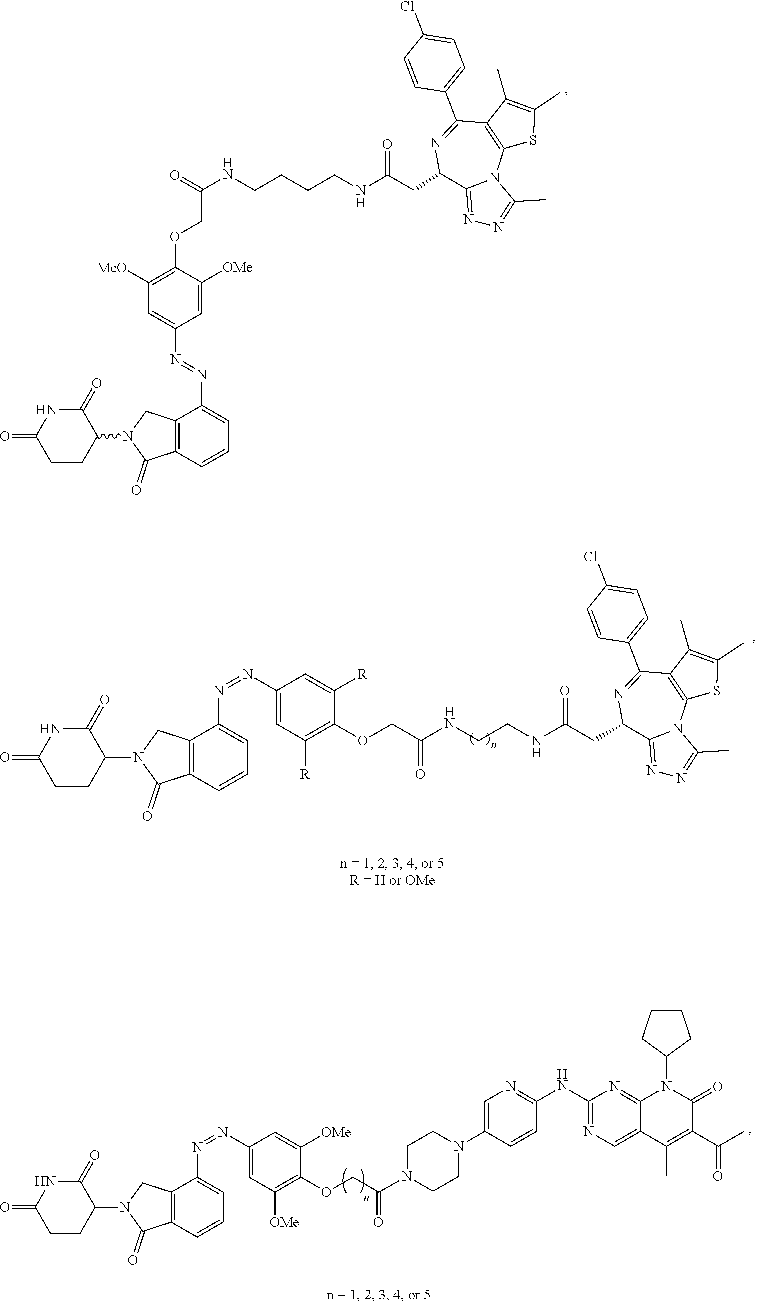

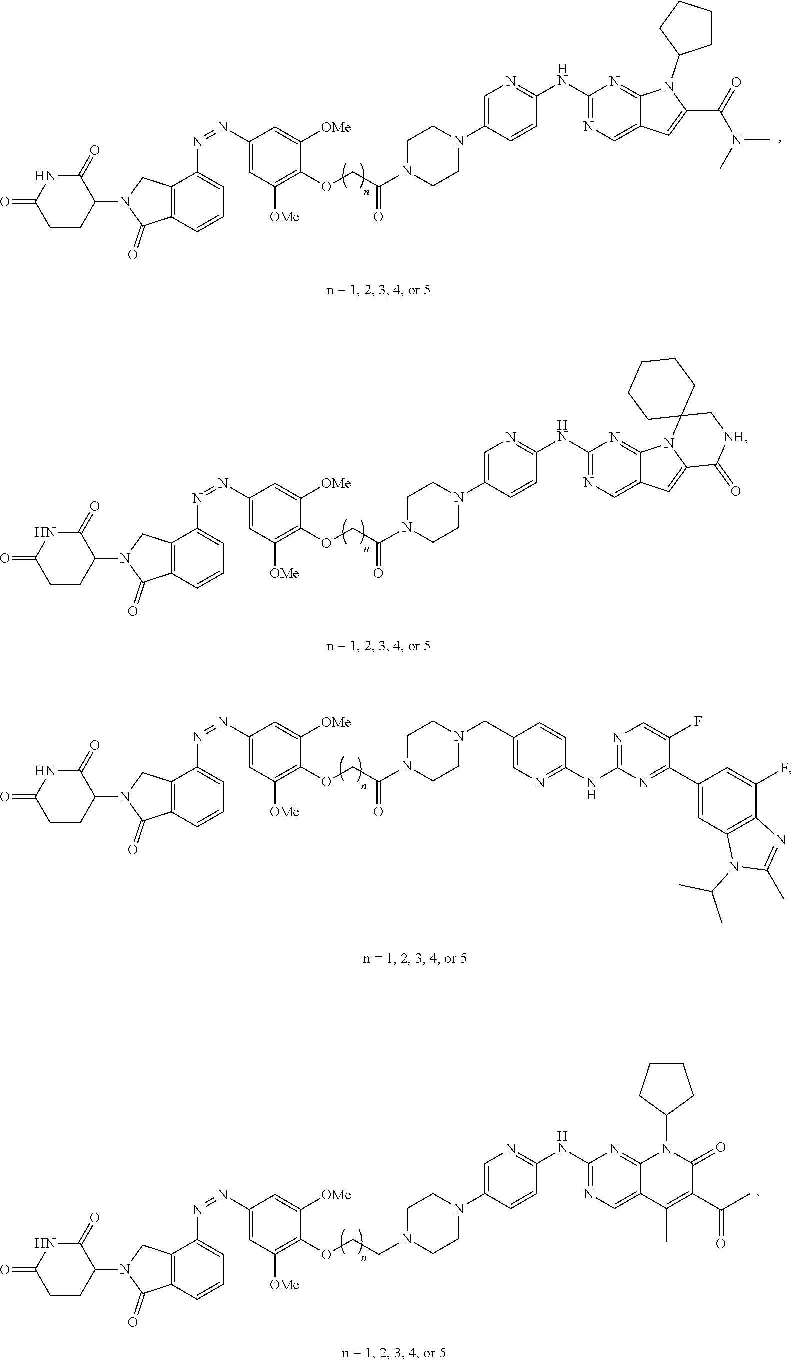

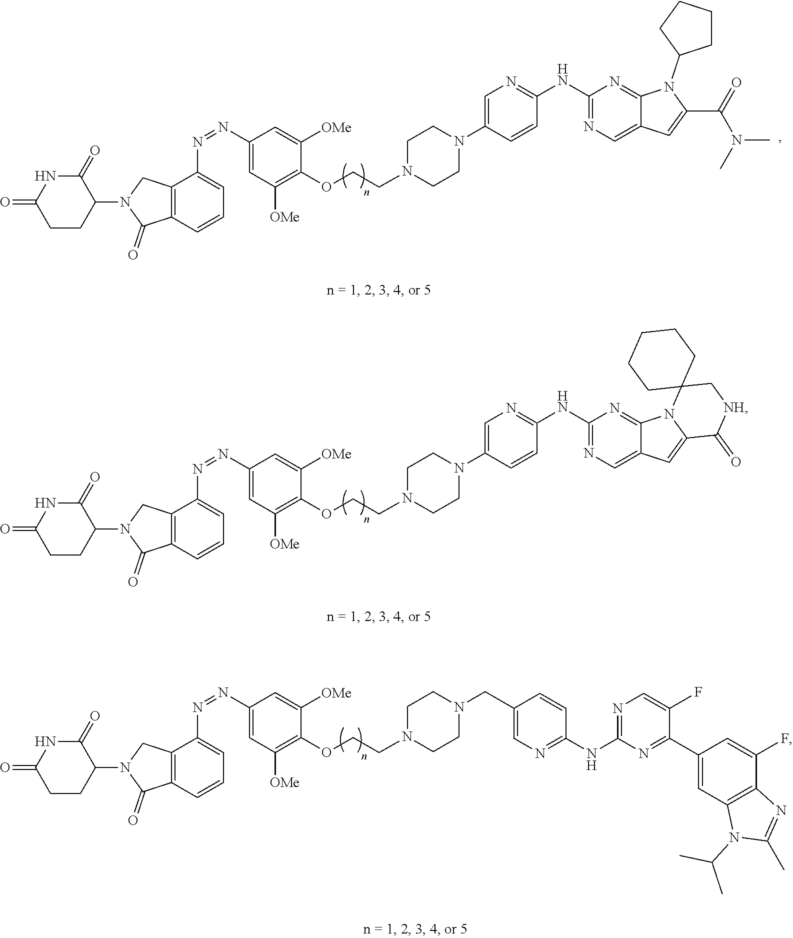

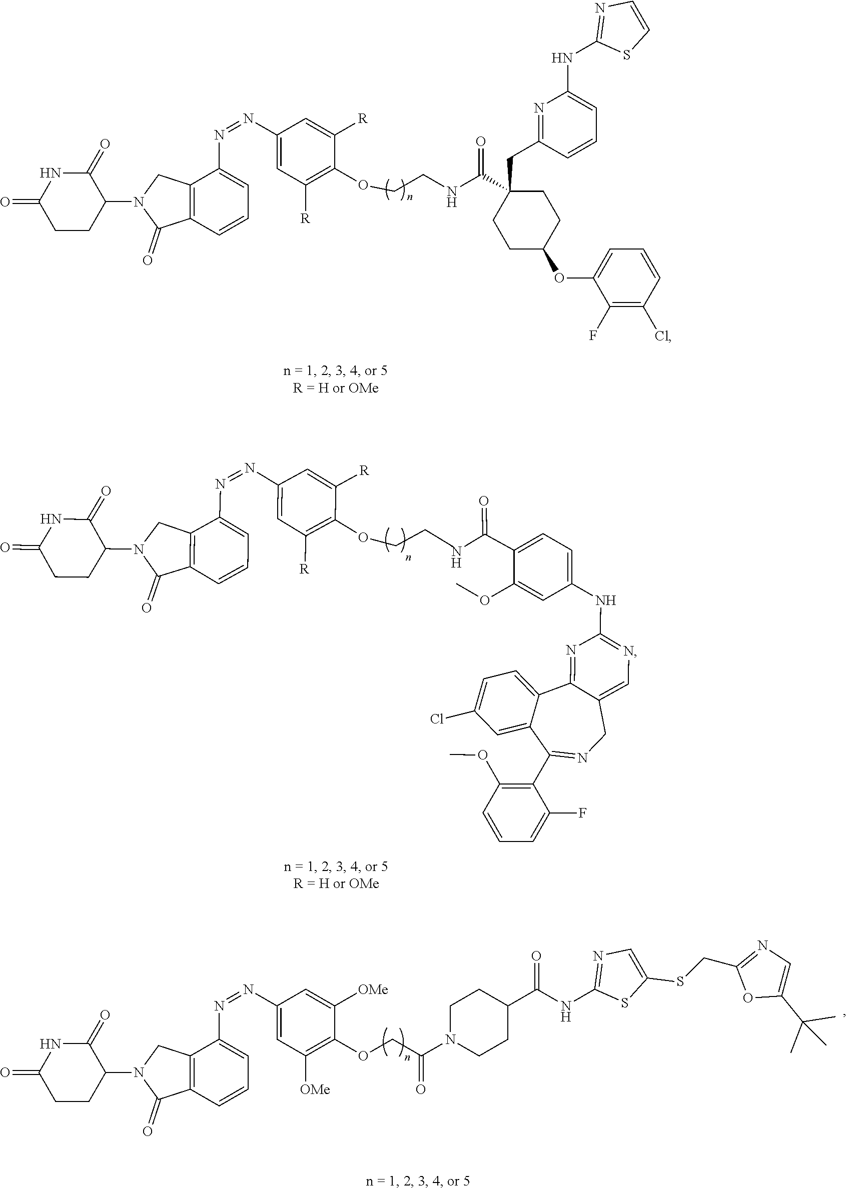

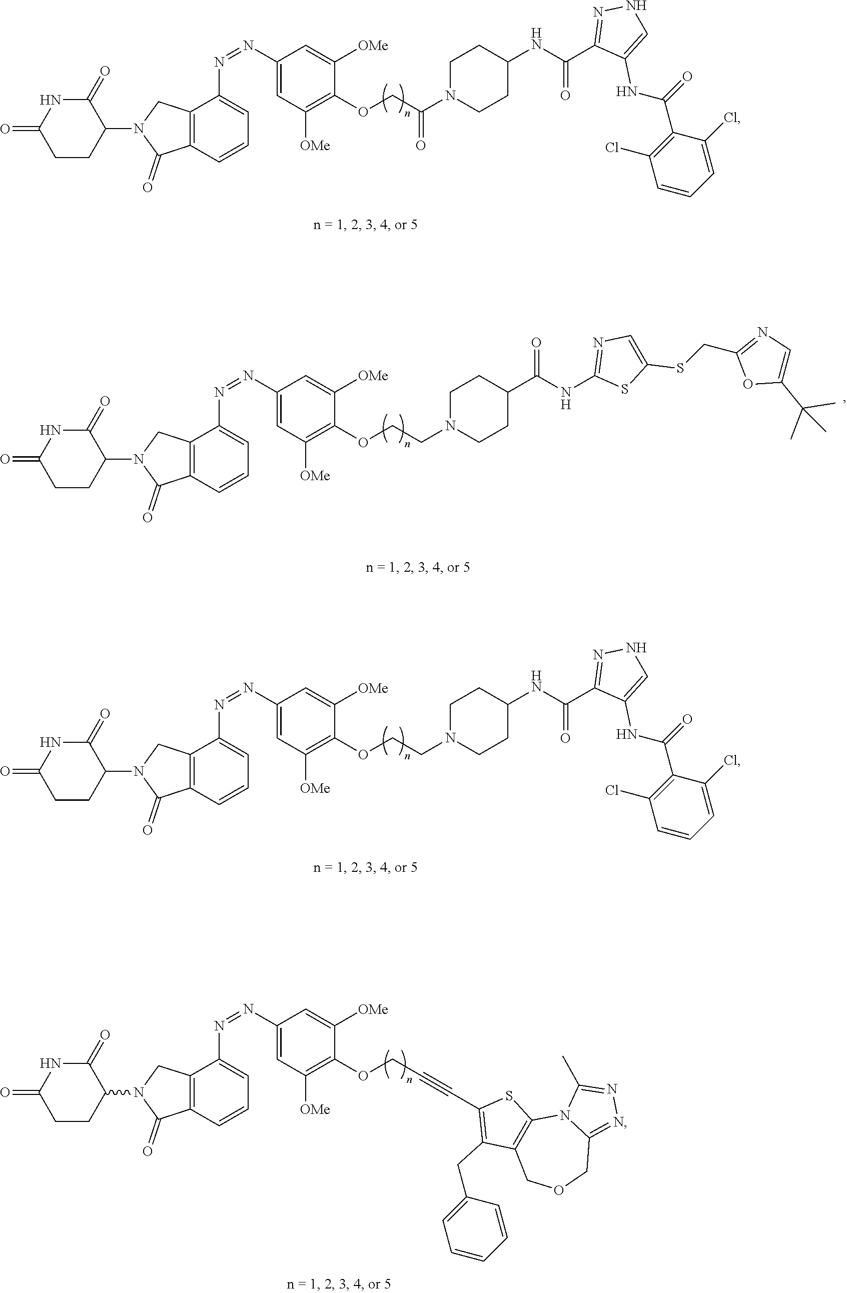

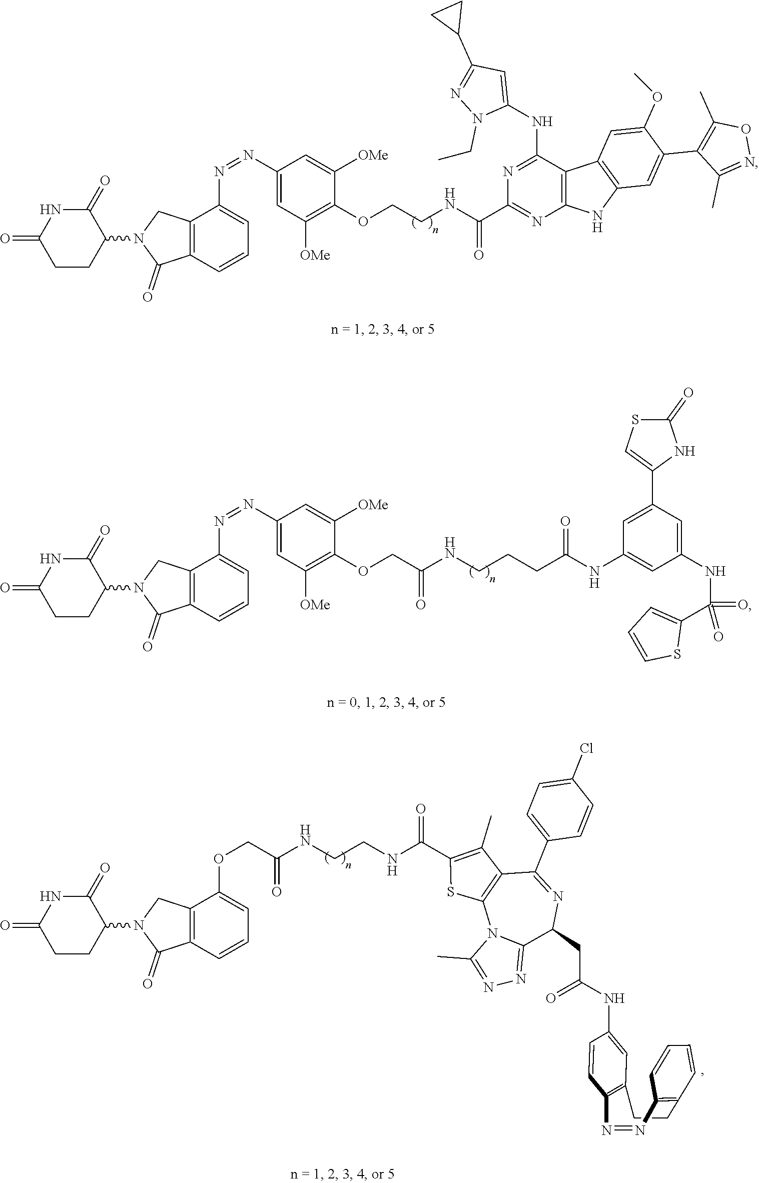

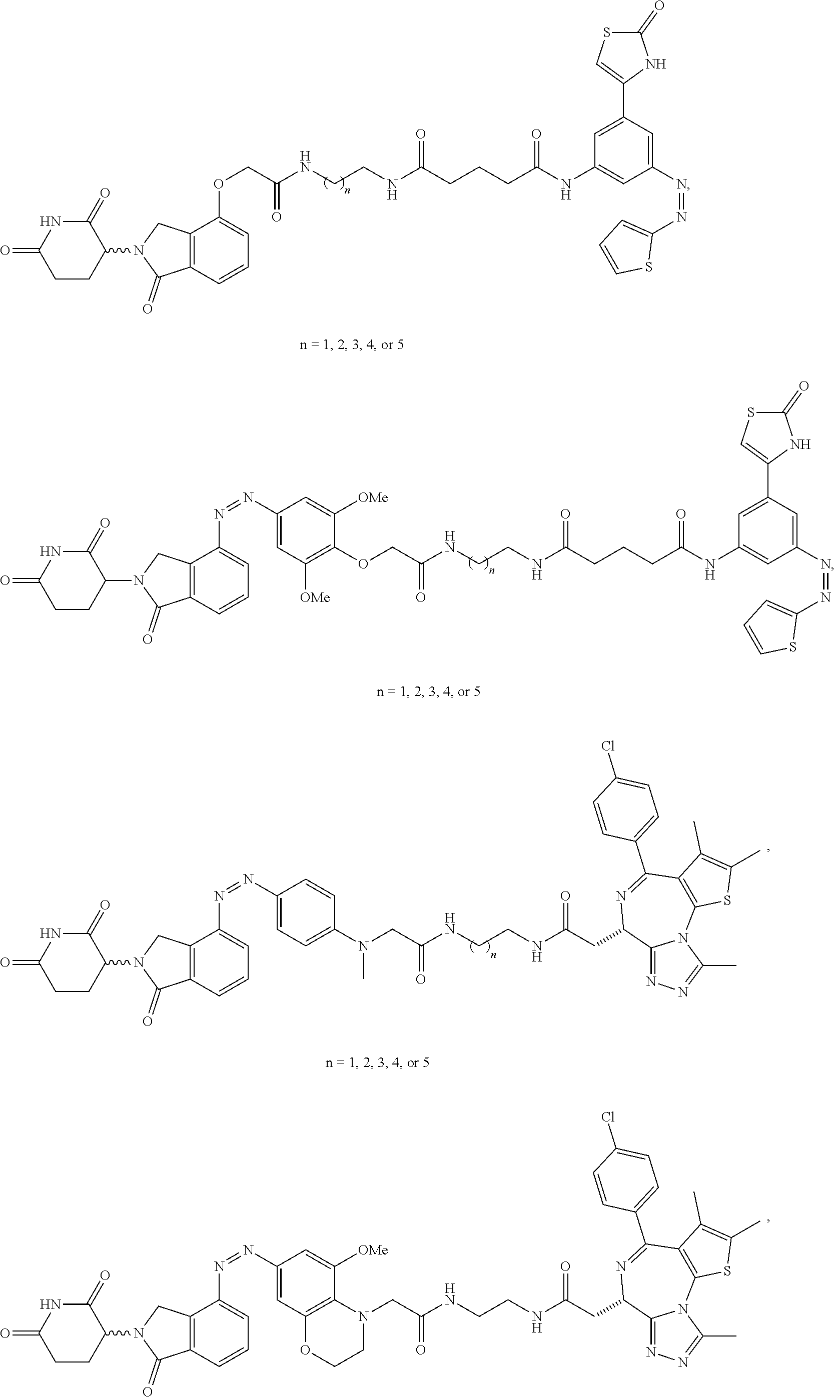

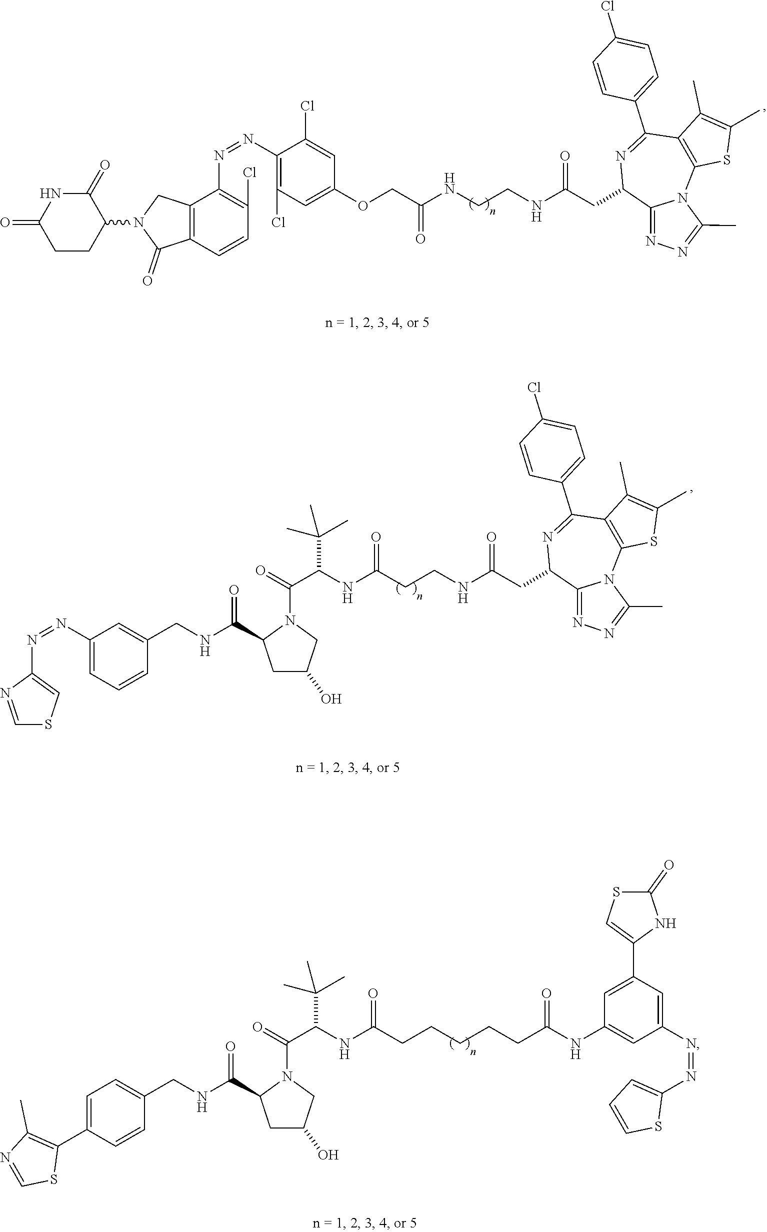

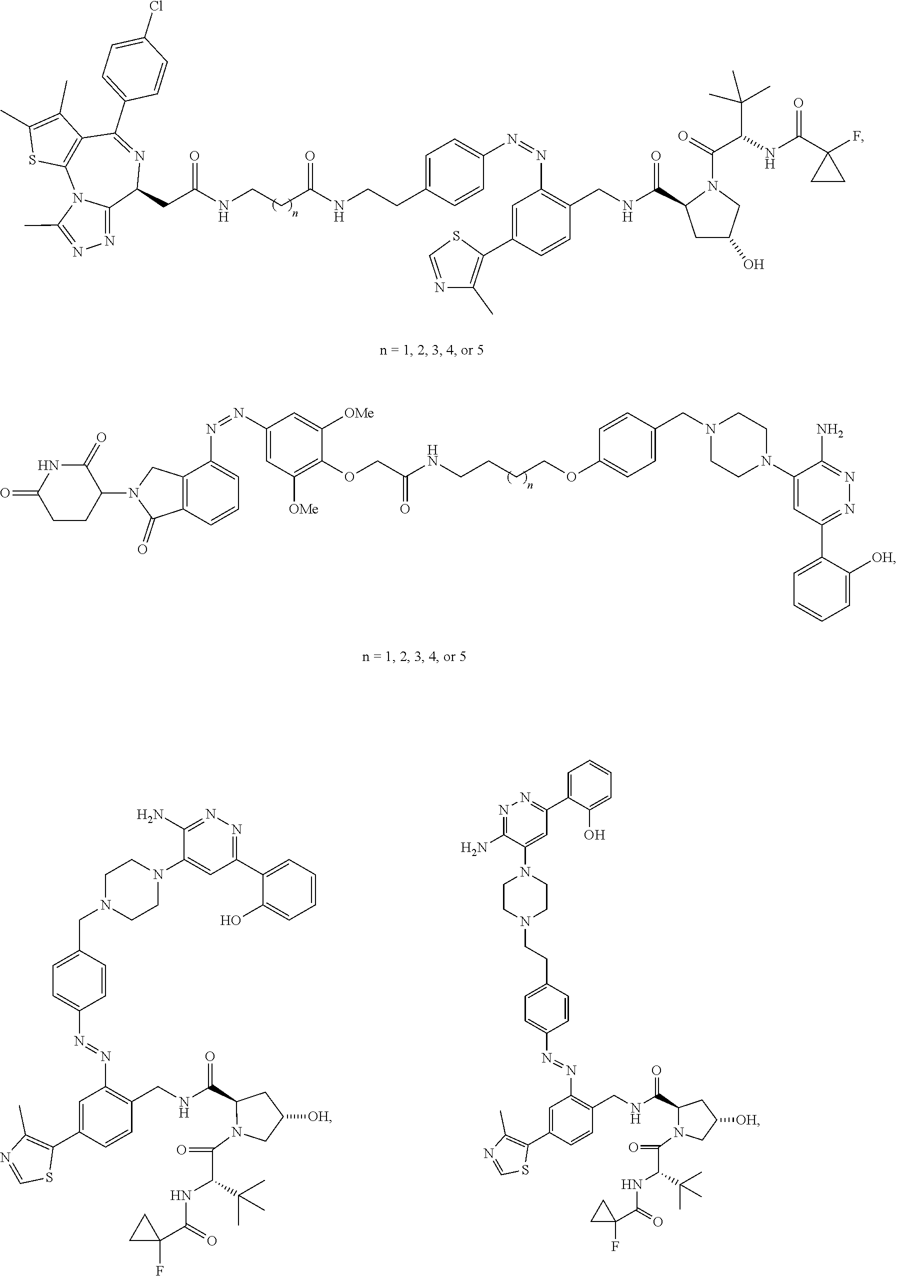

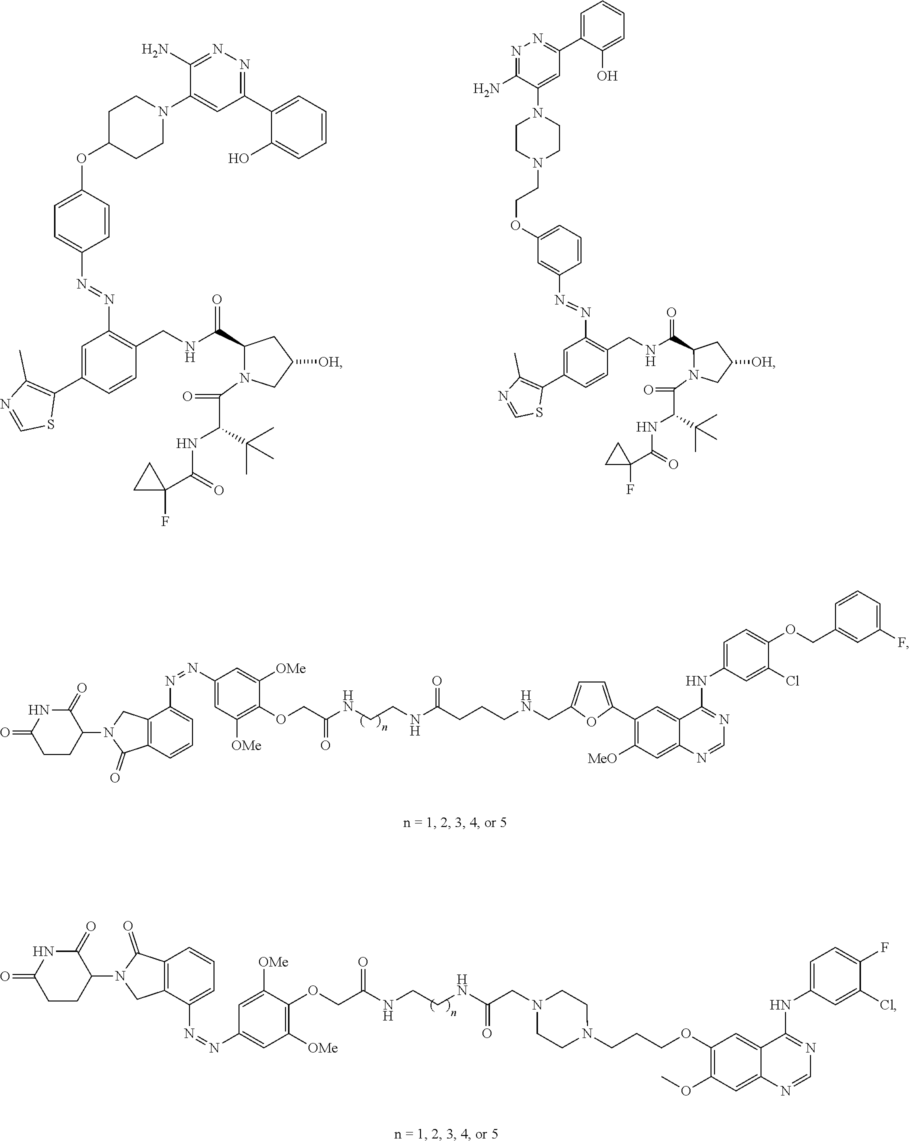

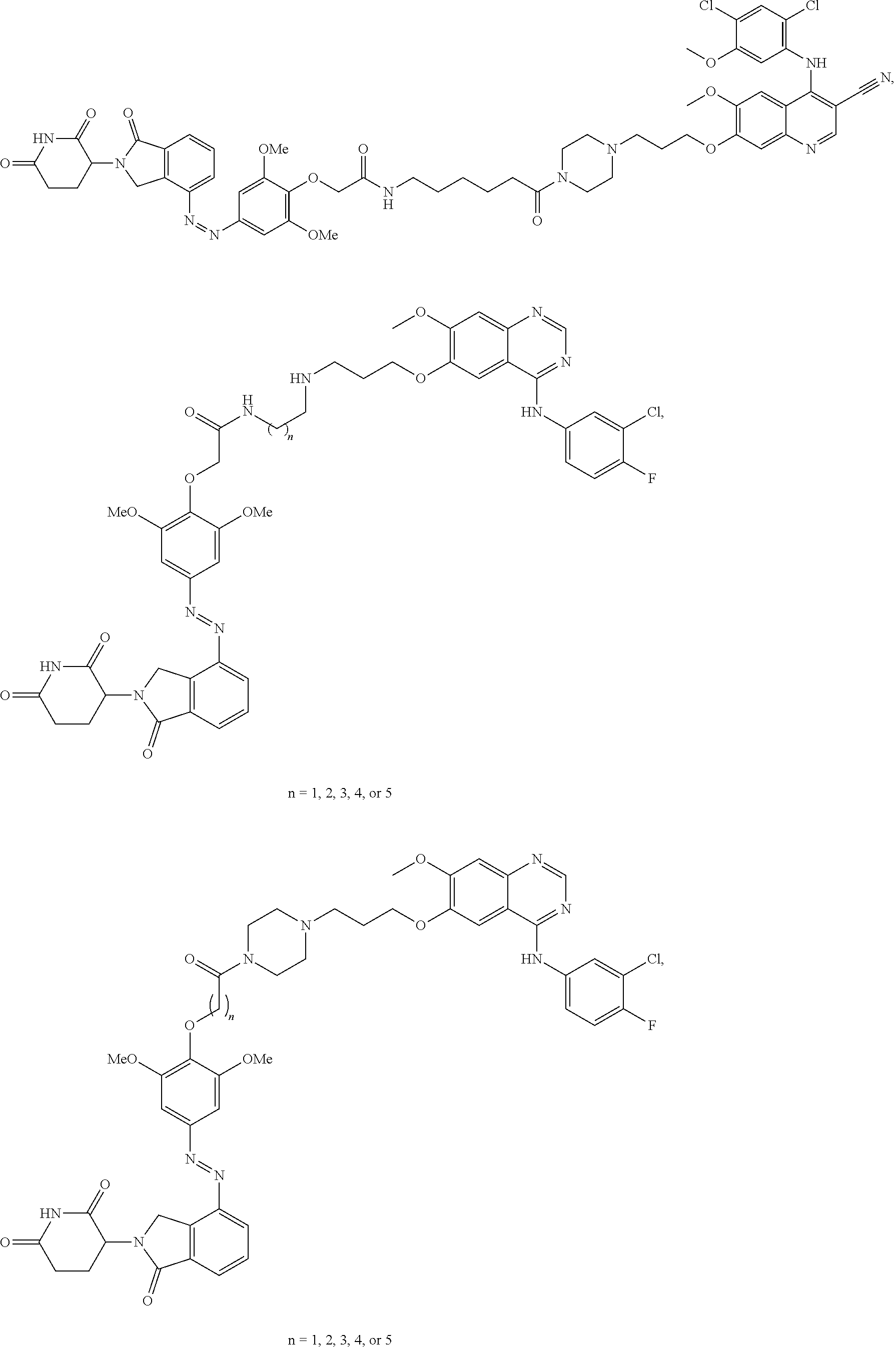

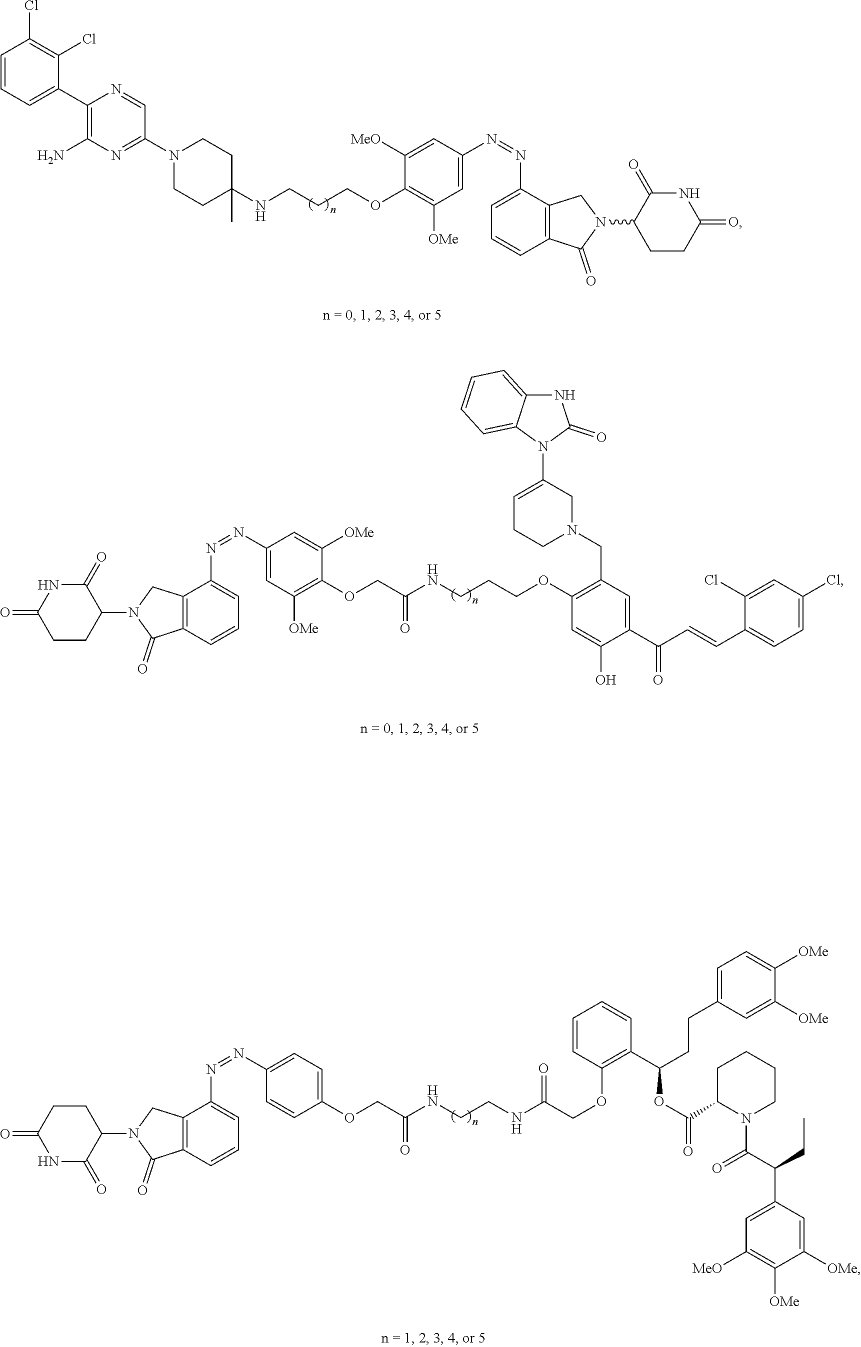

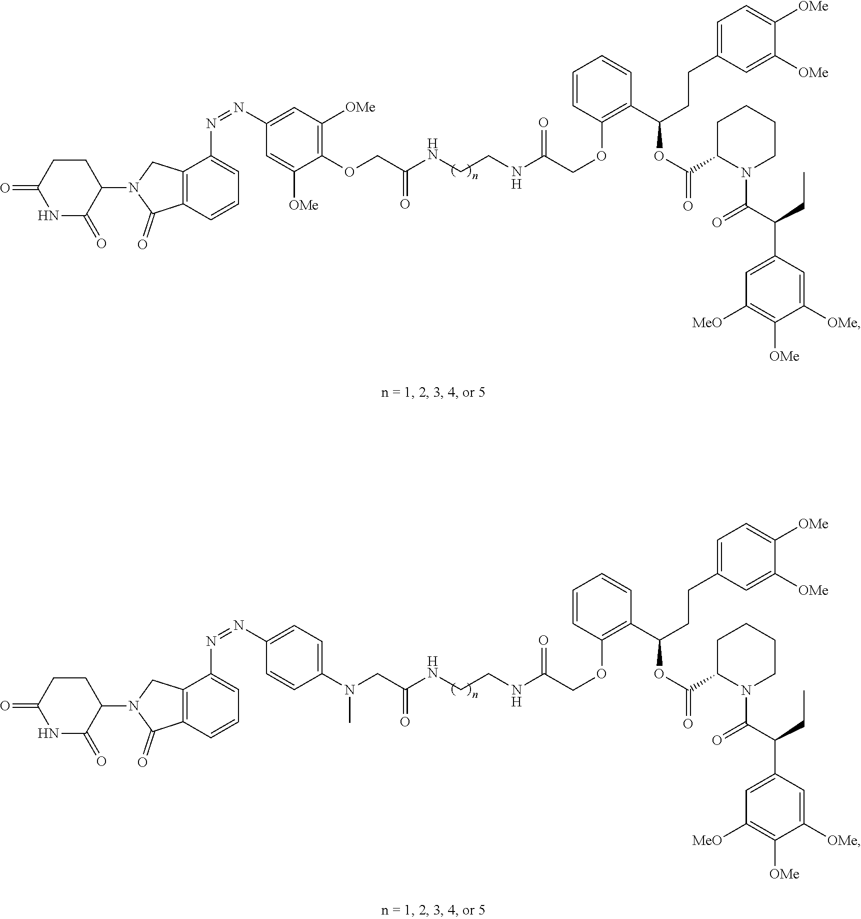

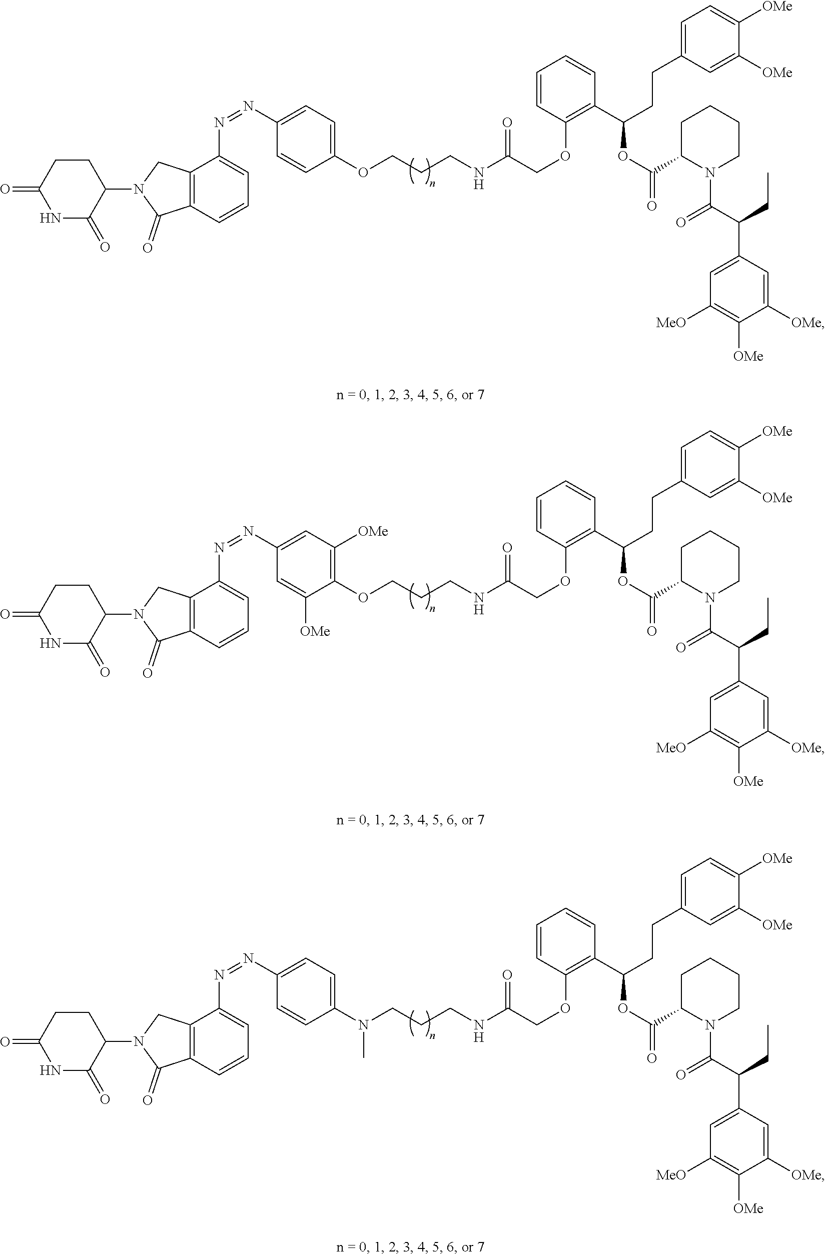

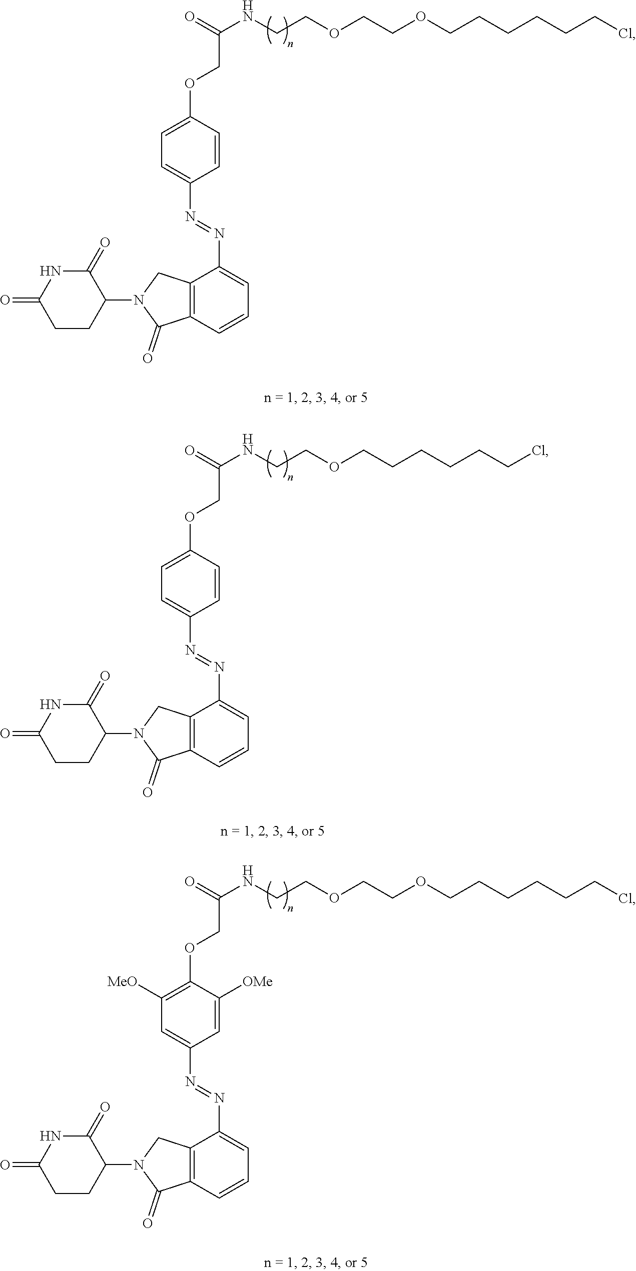

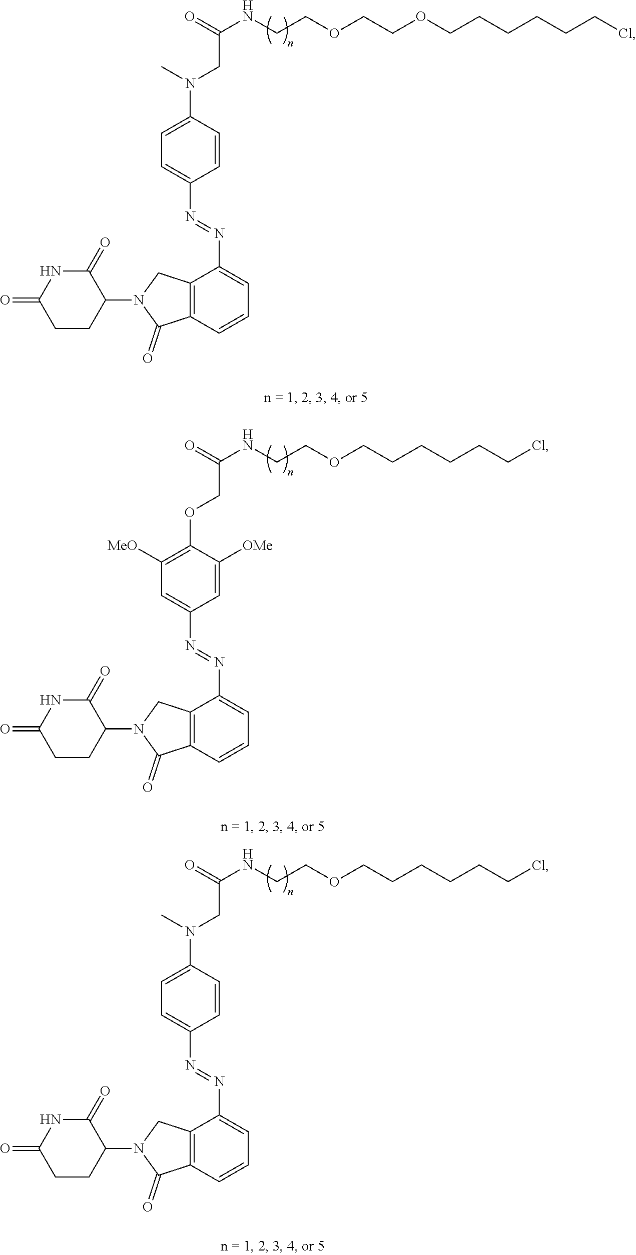

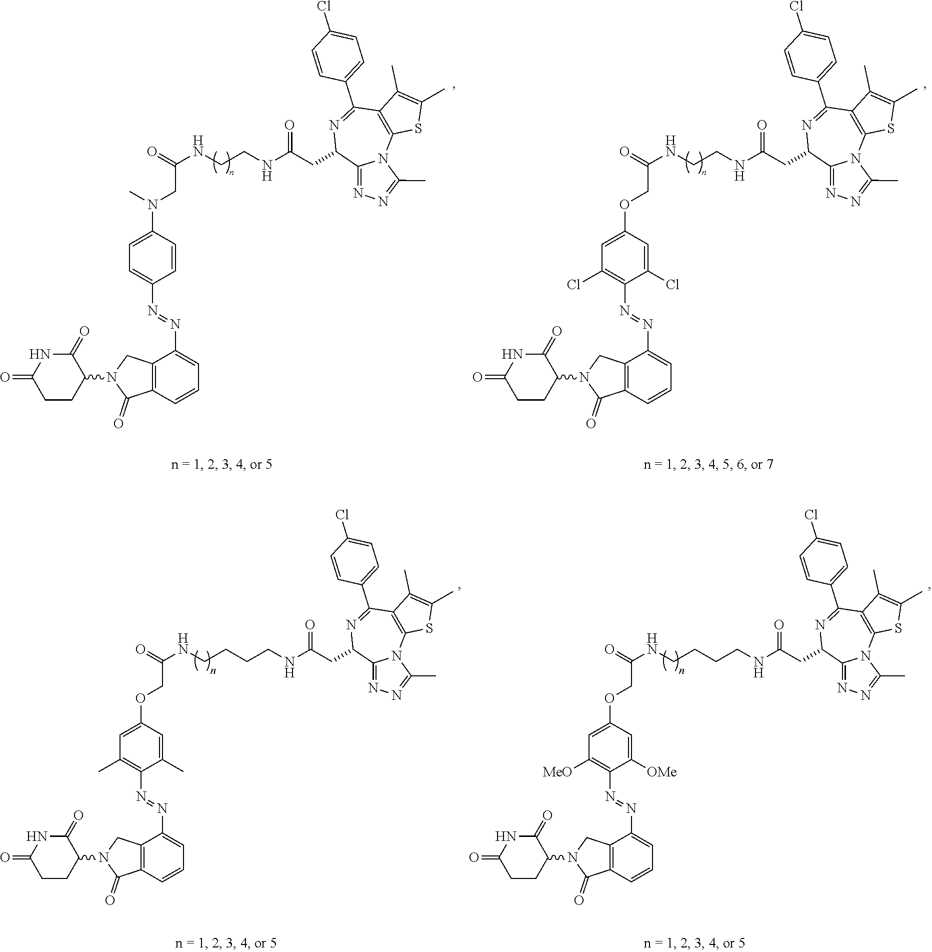

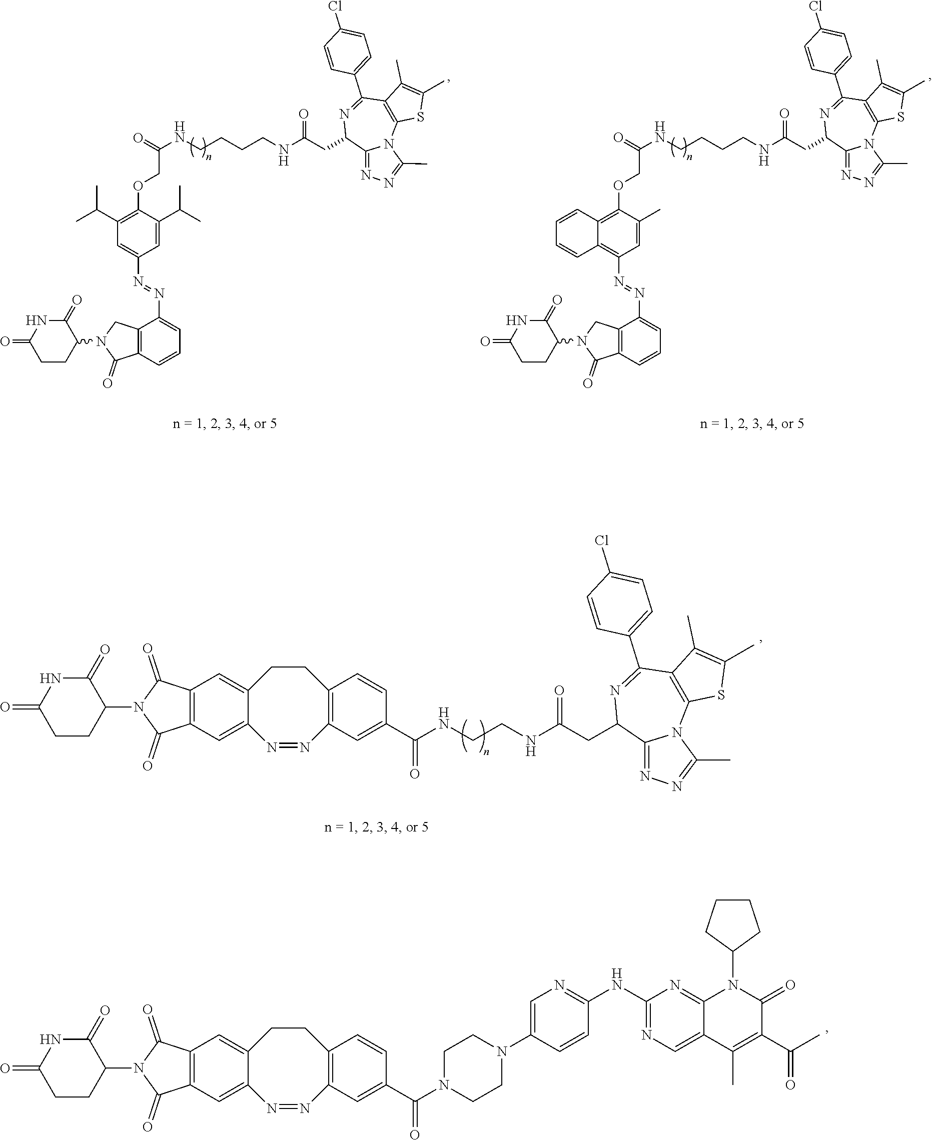

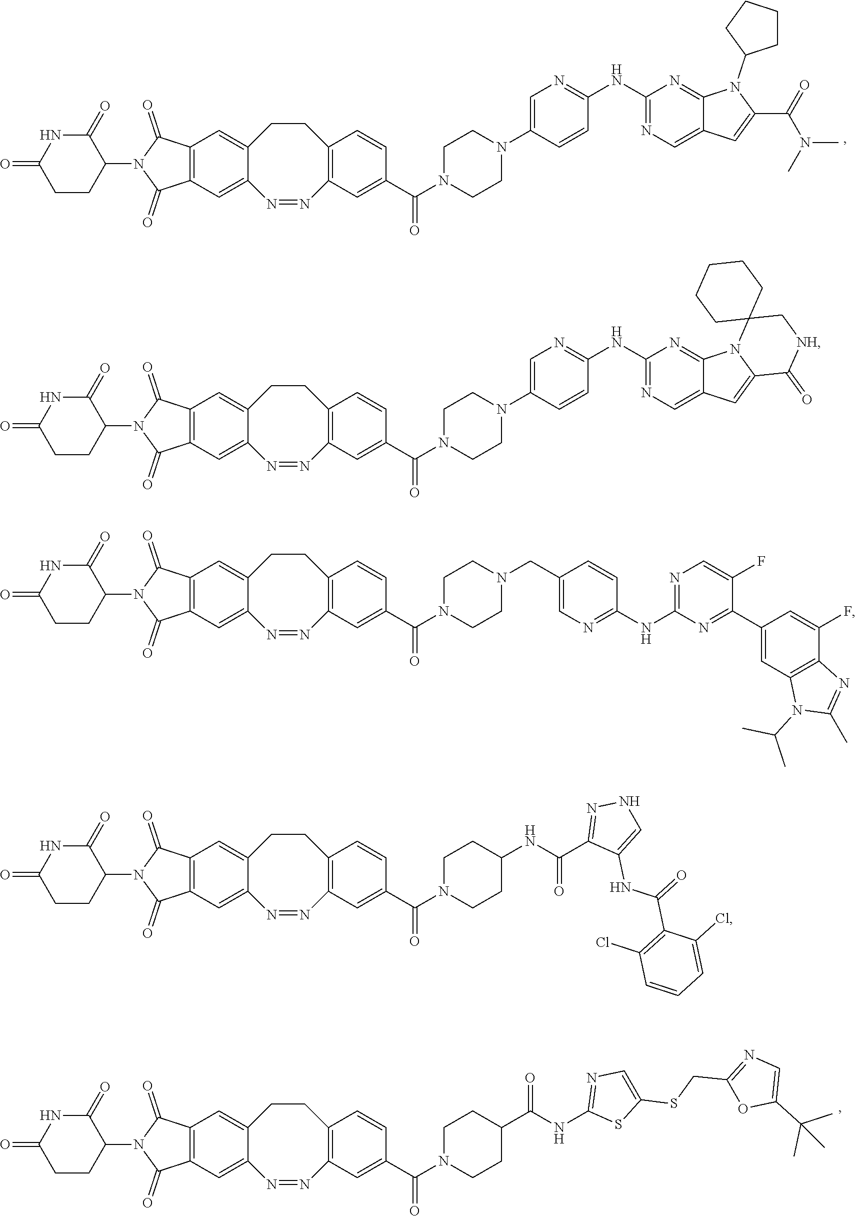

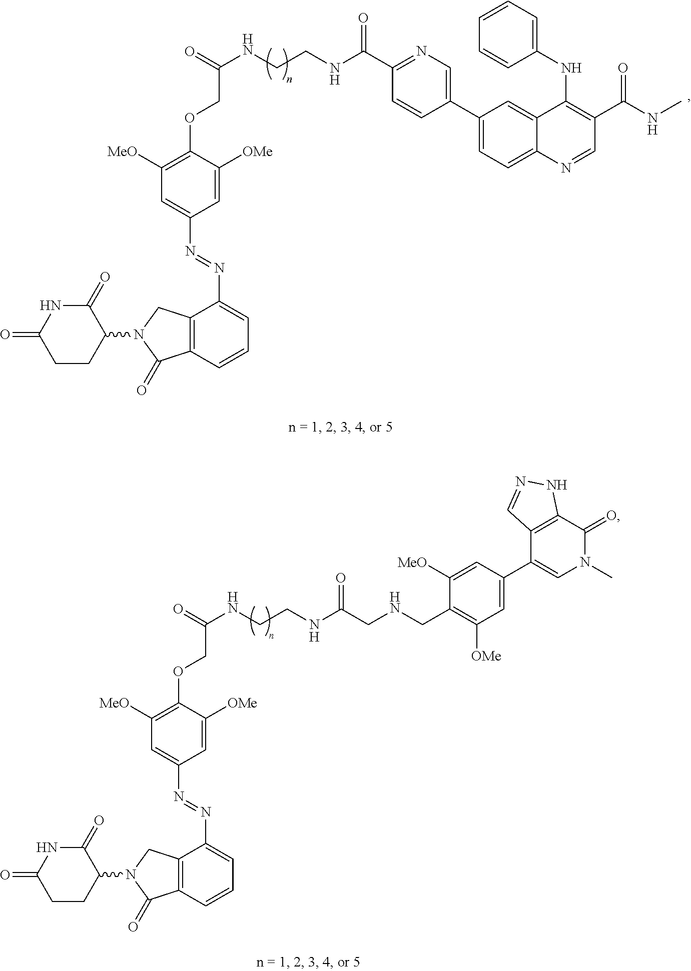

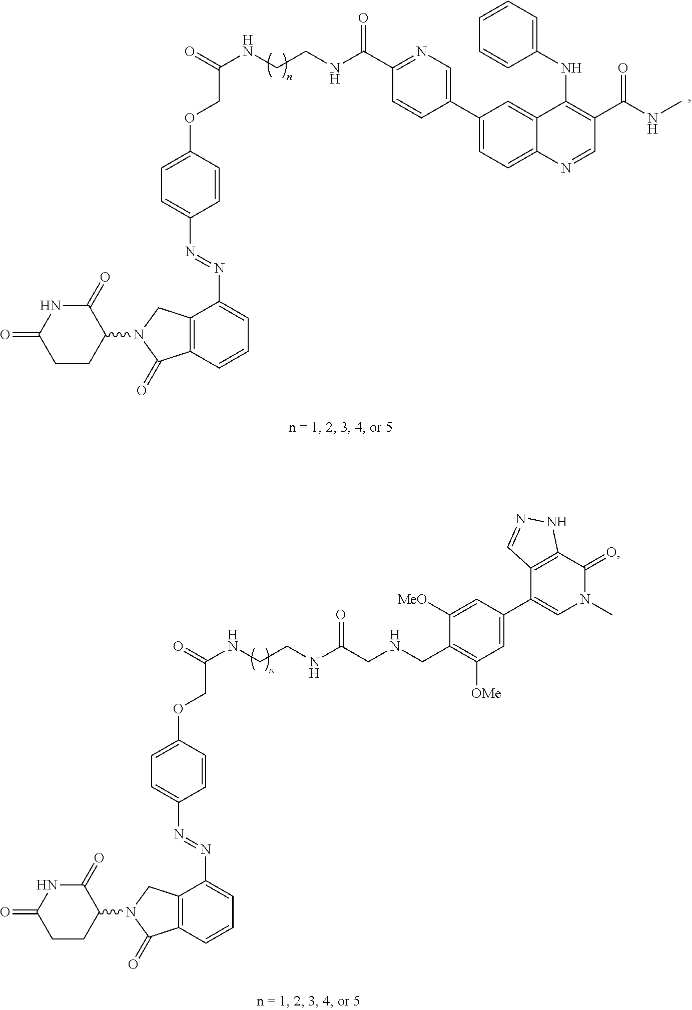

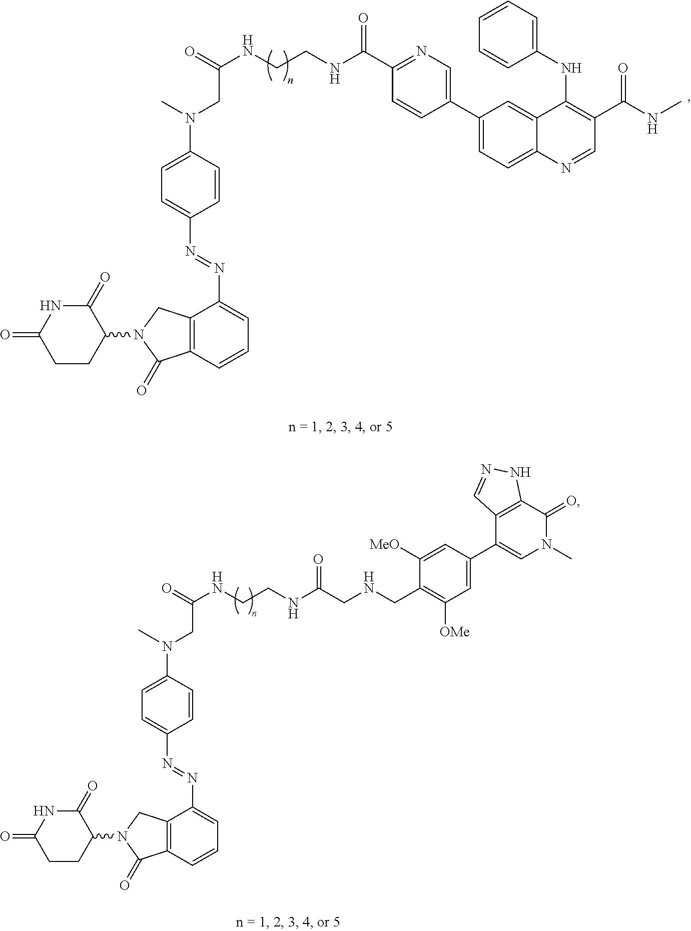

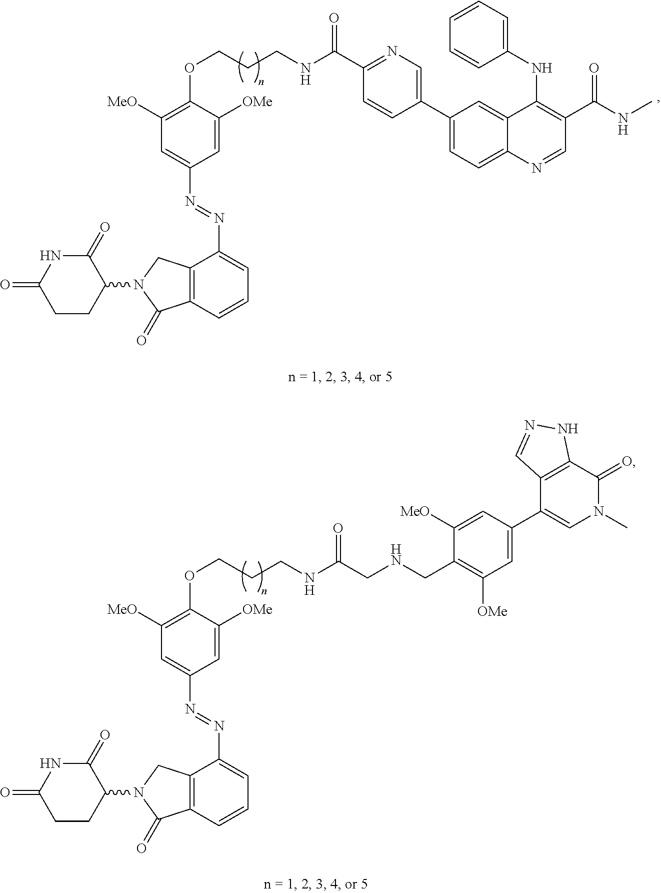

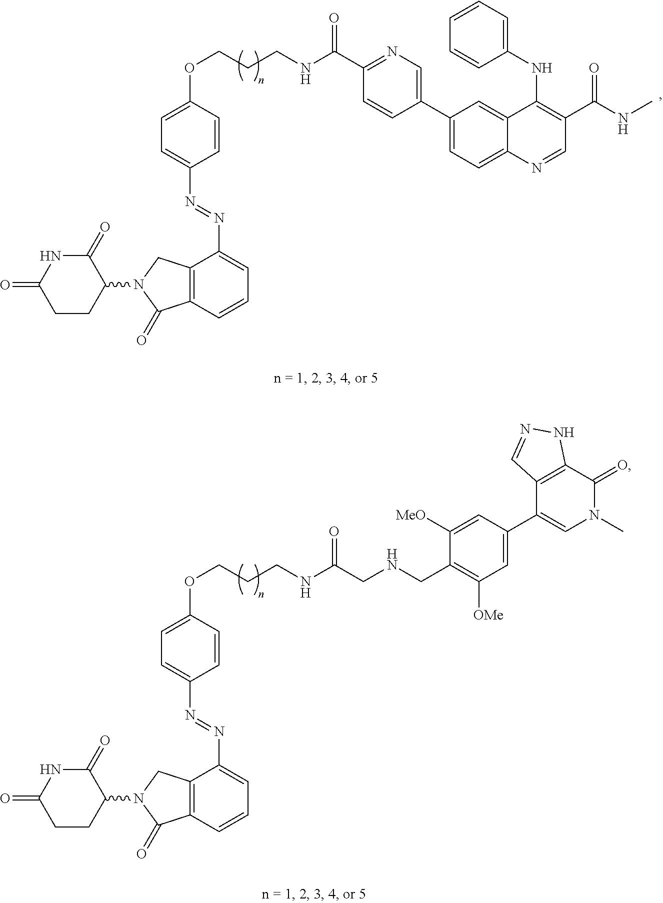

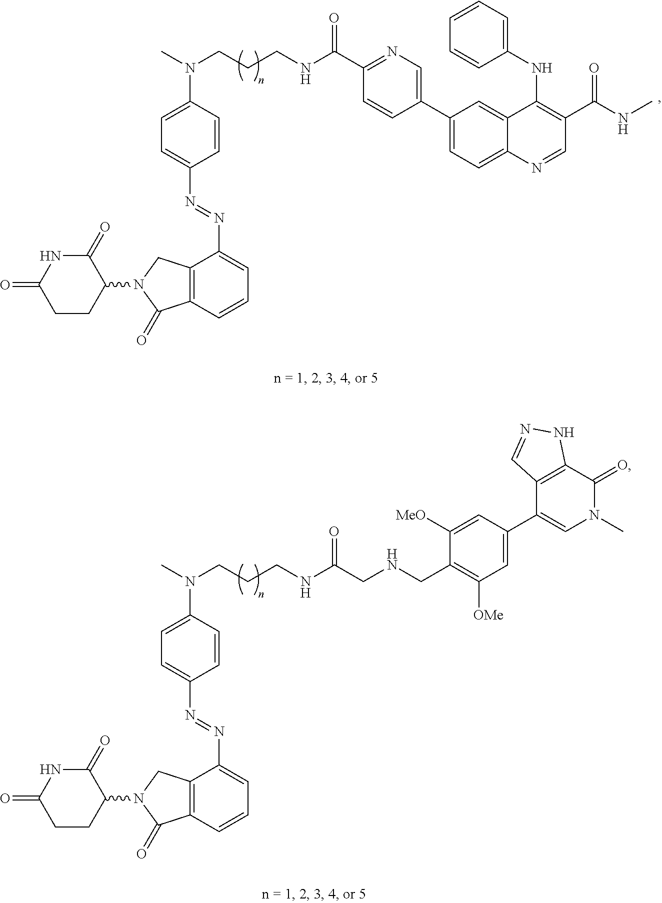

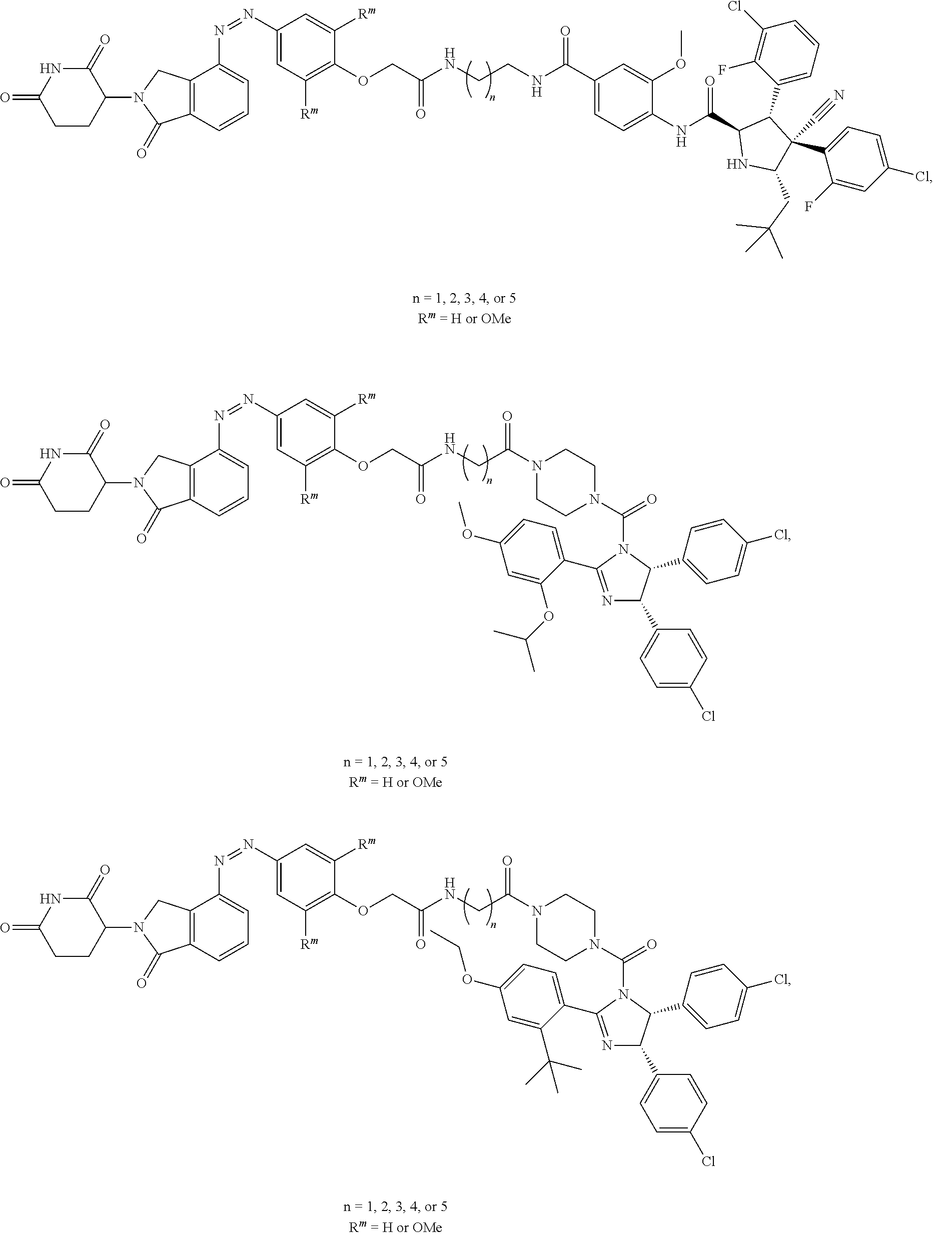

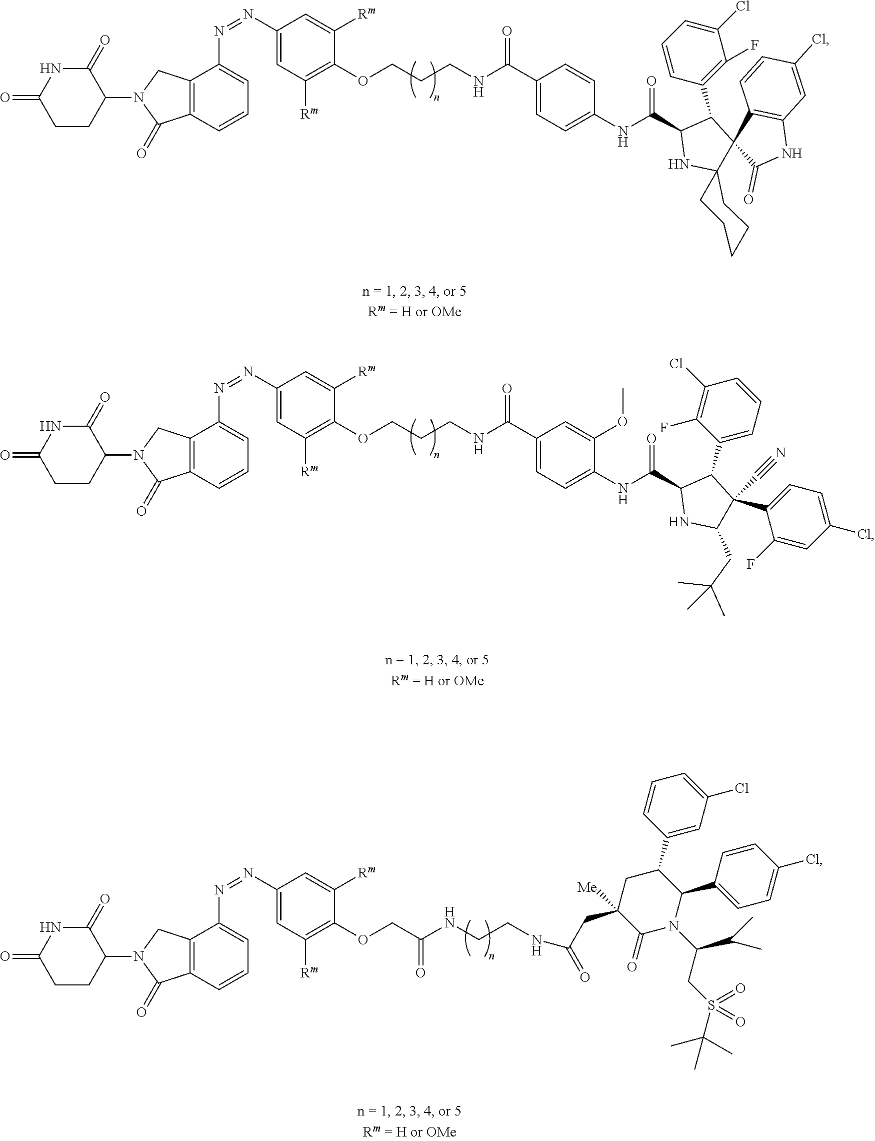

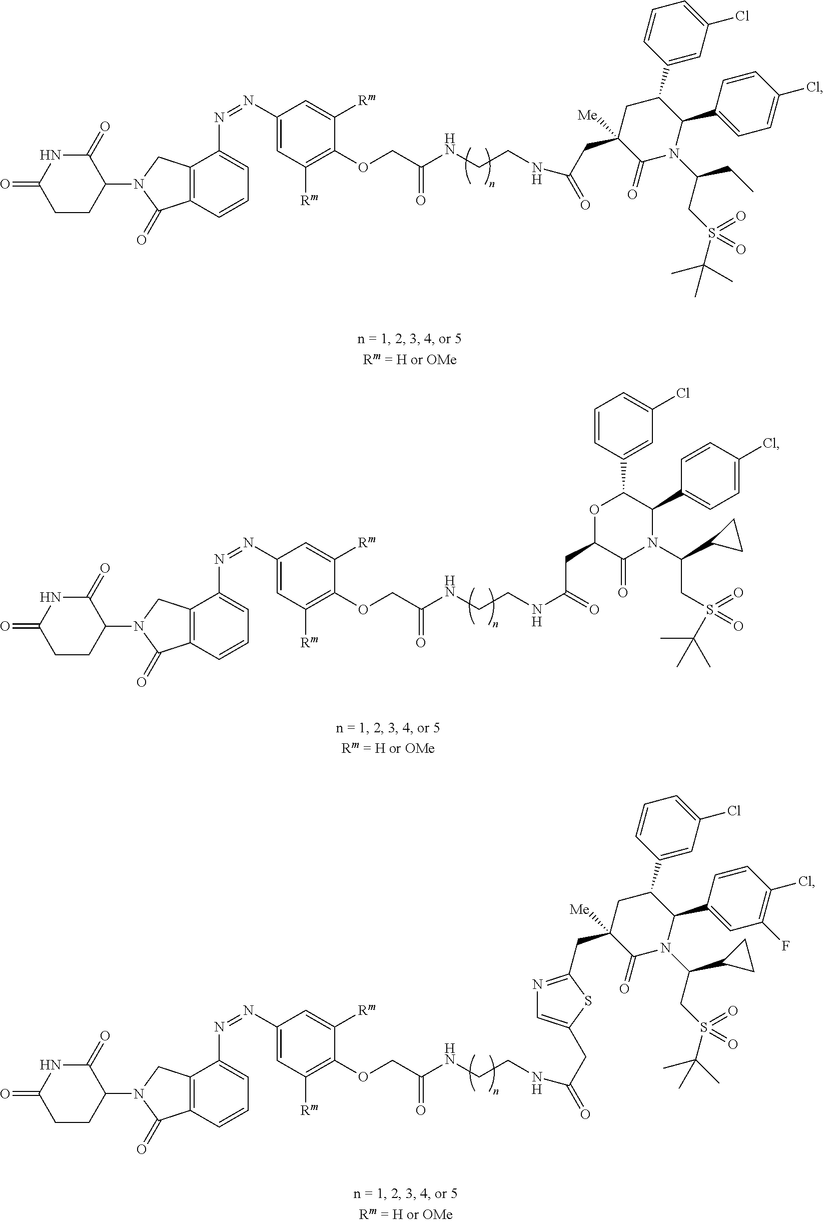

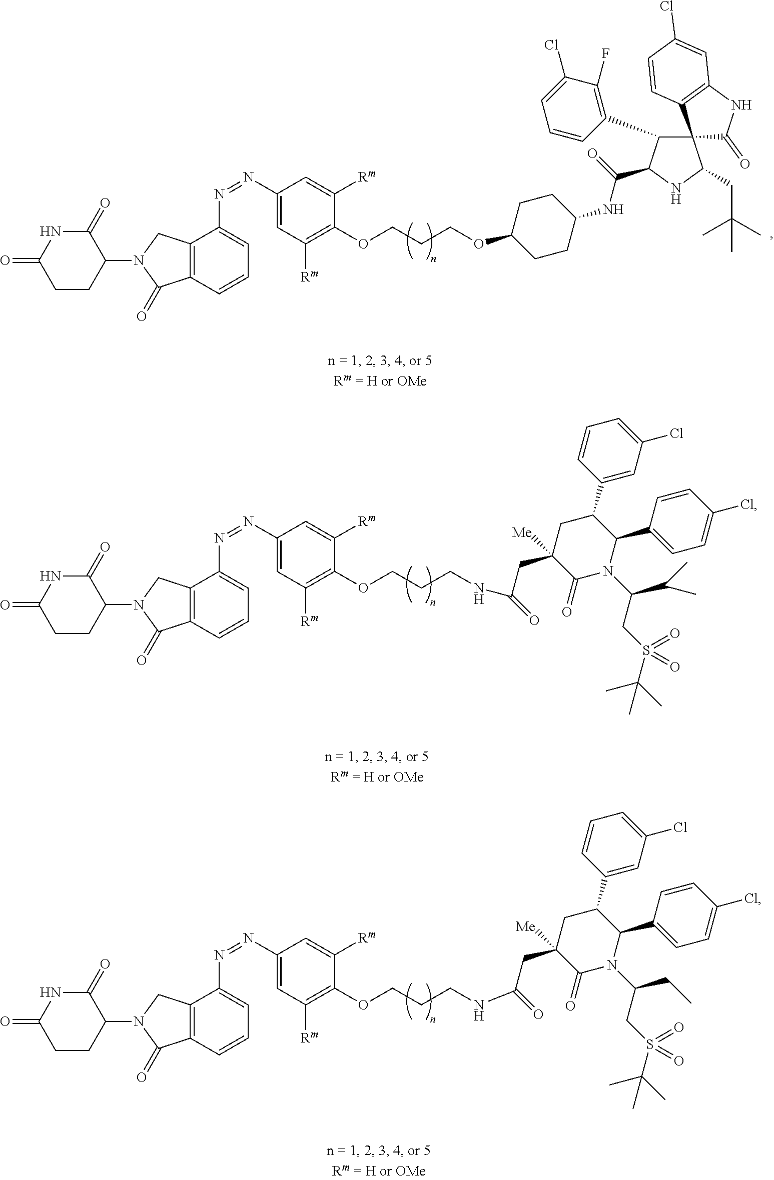

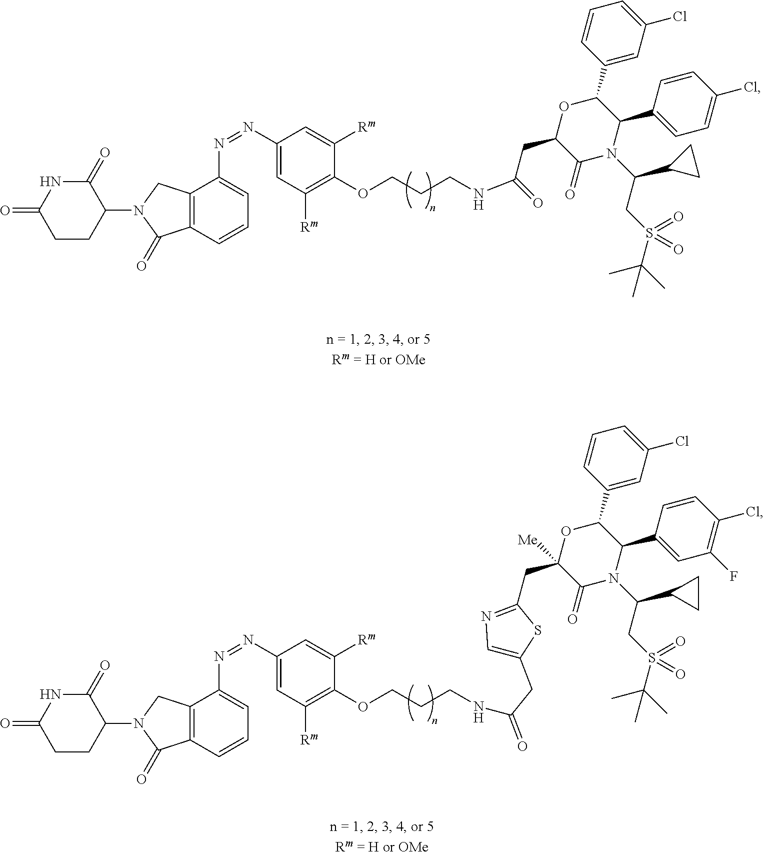

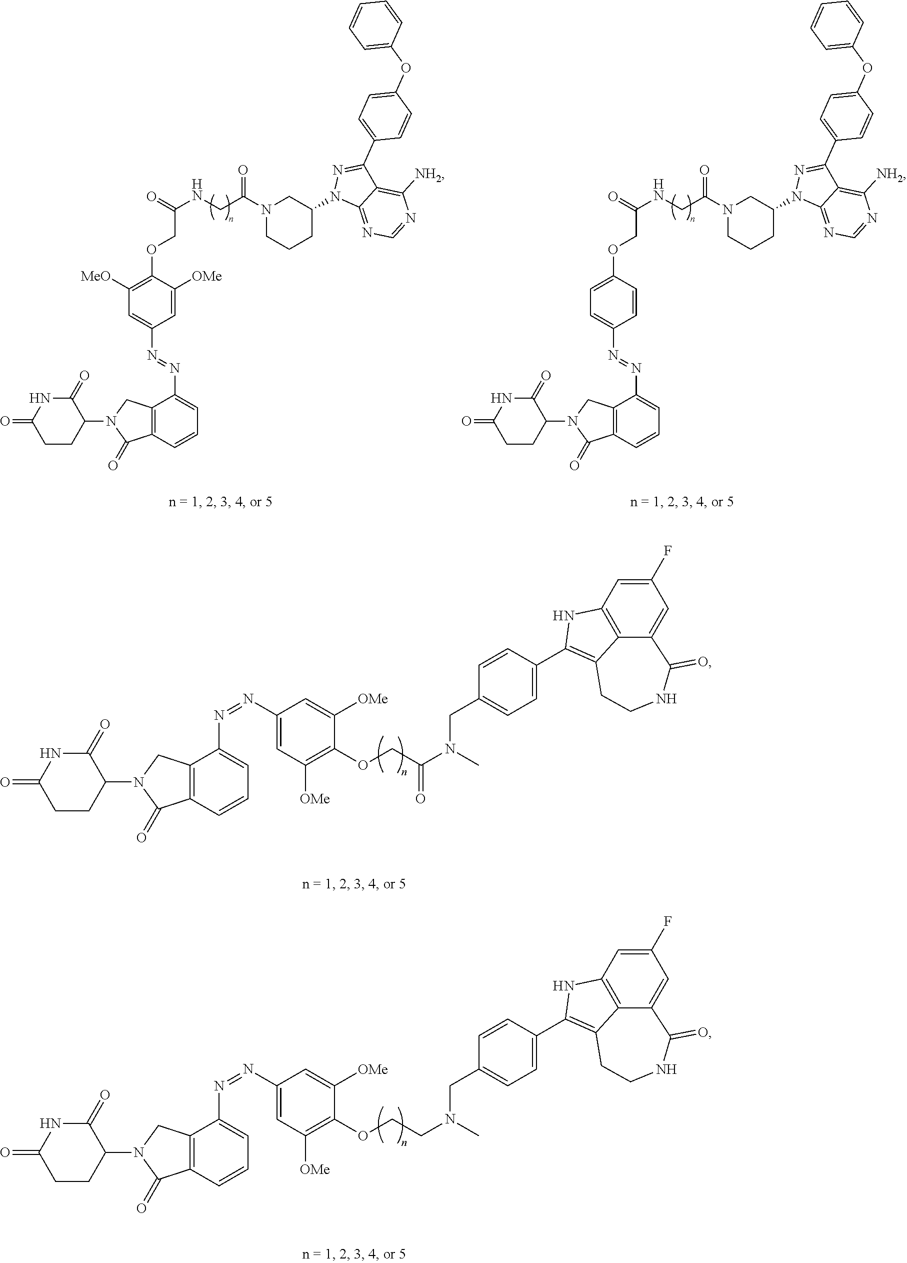

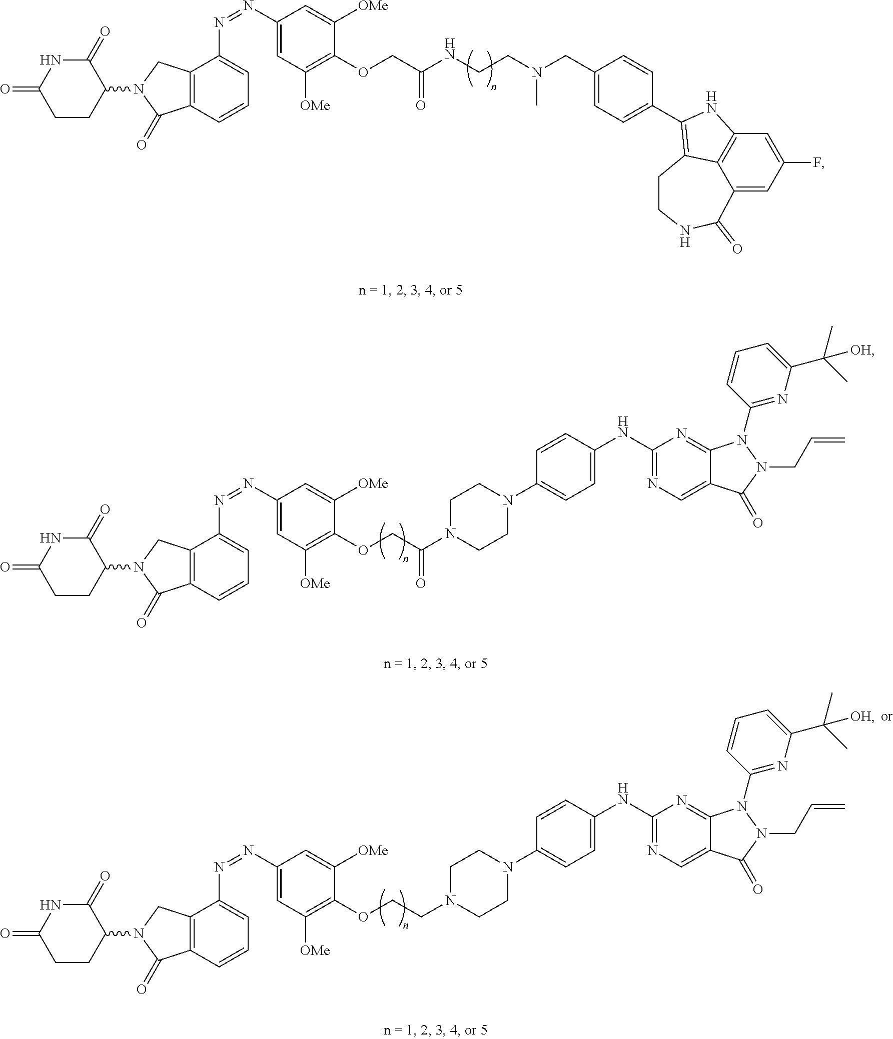

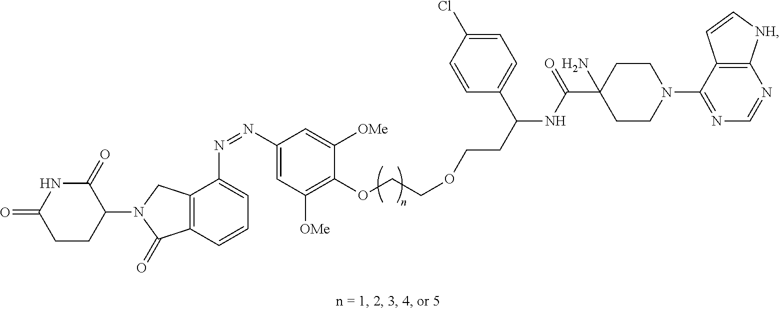

Statement 11. A composition comprising a compound according any one of the preceding Statements and a pharmaceutically acceptable carrier.
Statement 12. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in a deactivated conformation and the compound according to any one of Statements 1-10 binds to an E3 ligase; and exposing the cell or a portion thereof to electromagnetic radiation (e.g., light having a wavelength to induce an isomerization of the compound according to any one of Statements 1-10 to an activated conformation), where the exposing induces a conformational change in the compound according to any one of Statement 1-10 and the compound according to any one of Statements 1-10 binds to the target protein.

Statements 13. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in a deactivated conformation and the compound according to any one of Statements 1-10 binds to the target protein; and exposing the cell or a portion thereof to electromagnetic radiation (e.g., light having a wavelength to induce an isomerization of the compound according to any one of Statements 1-10 to an activated conformation), where the exposing induces a conformational change in the compound according to any one of Statements 1-10 and the compound according to any one of Statements 1-10 binds to an E3 ligase.

Statement 14. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in an activated conformation and the compound according to any one of Statements 1-10 binds to the target protein and an E3 ligase; and optionally, exposing the cell or a portion thereof to electromagnetic radiation (e.g., light having a wavelength to induce an isomerization of the compound according to any one of Statements 1-10 to a deactivated conformation), where the exposing induces a conformational change in the compound according to any one of Statements 1-10.
Statement 15. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in a deactivated conformation and the compound according to any one of Statements 1-10 binds to the target protein; waiting a period of time such that the compound of any one of Statements 1-10 relaxes to an activated state and binds to an E3 ligase.
Statement 16. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in a deactivated conformation and the compound according to any one of Statements 1-10 binds to an E3 ligase; waiting a period of time such that the compound of any one of Statements 1-10 relaxes to an activated state and binds to the target protein.
Statement 17. A method of inducing selective degradation of a target protein in a cell, comprising: contacting a cell or cells (e.g., a cell or cells in a subject in need of treatment) with a compound according to any one of Statements 1-10 and/or a composition of Statement 11, where the compound according to any one of Statements 1-10 is in an activated conformation and such that the compound according to any one of Statements 1-10 binds to the target protein and an E3 ligase; waiting a period of time such that the compound of any one of Statements 1-10 relaxes to a deactivated state.
Statement 18. A method of inducing selective degradation of a target protein in a cell according to any one of Statements 12-14, where the method further comprises exposing the cell or cells (e.g., a cell or cells in a subject in need of treatment) to electromagnetic radiation (e.g., light having a wavelength of 375-405 nm (such as, for example, 390 nm) or 485-515 nm (such as, for example, 500 nm)) one or more additional time(s) after the exposing of any one of Statements 12-14.
Statement 19. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof), comprising: administering to a subject in need of treatment a composition of Statement 11, where the compound according to any one of Statements 1-10 is in a deactivated conformation and the compound according to any one of Statements 1-10 binds to an E3 ligase; and exposing the subject in need of treatment or a portion thereof to electromagnetic radiation (e.g., light having a wavelength to induce an isomerization of the compound according to any one of Statements 1-10 to an activated conformation), where the exposing induces a conformational change in the compound according to any one of Statement 1-10 and the compound according to any one of Statement 1-10 binds to a target protein.
Statement 20. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof), comprising: administering to a subject in need of treatment a composition of Statement 11, where the compound according to any one of Statements 1-10 is in a deactivated conformation and the compound according to any one of Statements 1-10 binds to a target protein; and exposing the subject in need of treatment or a portion thereof to electromagnetic radiation (e.g., light having a wavelength to induce an isomerization of the compound according to any one of Statements 1-10 to an activated conformation), where the exposing induces a conformational change in the compound according to any one of Statements 1-10 and the compound according to any one of Statements 1-10 binds to an E3 ligase.
Statement 21. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof), comprising: administering to a subject in need of treatment (e.g., a cell or cells in a subject in need of treatment) a composition of Statement 11, where the compound according to any one of Statements 1-10 is in an activated conformation and the compound according to any one of Statements 1-10 binds to a target protein and an E3 ligase; and optionally, exposing the cell or cells or a portion thereof to electromagnetic radiation (e.g., light having a wavelength to induce an isomerization of the compound according to any one of Statements 1-10 to a deactivated conformation); where the exposing induces a conformational change in the compound according to any one of Statements 1-10.
Statement 22. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof), comprising: administering to a subject in need of treatment (e.g., a cell or cells in a subject in need of treatment) a composition of Statement 11, where the compound according to any one of Statements 1-10 is in a deactivated conformation and the compound according to any one of Statements 1-10 binds to the target protein; waiting a period of time such that the compound of any one of Statements 1-10 relaxes to an activated state and binds to an E3 ligase.
Statement 23. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combination thereof), comprising: administering to a subject in need of treatment (e.g., a cell or cells in a subject in need of treatment) a composition of Statement 11, where the compound according to any one of Statements 1-10 is in a deactivated conformation and the compound according to any one of Statements 1-10 binds to an E3 ligase; waiting a period of time such that the compound of any one of Statements 1-10 relaxes to an activated state and binds to the target protein.
Statement 24. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof), comprising: administering to a subject in need of treatment (e.g., a cell or cells in a subject in need of treatment) a composition of Statement 11, where the compound according to any one of Statements 1-10 is in an activated conformation and such that the compound according to any one of Statements 1-10 binds to a target protein and an E3 ligase; waiting a period of time such that the compound of any one of Statements 1-10 relaxes to a deactivated state.
Statement 25. A method of treating cancer (e.g., leukemia, lung cancer, dermatological cancers, premalignant lesions of the upper digestive tract, malignancies of prostate, brain, breast, and the like, and combinations thereof) or other disease (e.g., infectious diseases, inflammatory diseases, immune disorders, sleep disorders, neurodegenerative disorders, and the like, and combinations thereof) according to any one of Statements 19-21, where the method further comprises exposing the subject in need of treatment (e.g., a cell or cells in a subject in need of treatment) to electromagnetic radiation (e.g., light having a wavelength of 375-405 nm (such as, for example, 390 nm) or 485-515 nm (such as, for example, 500 nm)) one or more additional time(s) after the exposing of any one of Statements 19-21.

The following example is presented to illustrate the present disclosure. It is not intended to be limiting in any matter.

### Example 1

This example describes PHOTACs of the present disclosure.

One method to localize the effect of drugs and achieve higher selectivity is to control their activity with light. In recent years, the usefulness of light to precisely regulate biological pathways has become increasingly apparent. Optical control can be achieved in a variety of ways: with caged compounds, genetically engineered photoreceptors

(Optogenetics), or with synthetic photoswitches whose activity can be changed through a combination of photochemical isomerization and thermal relaxation (Photopharmacology).

Described herein are the application of photopharmacology to targeted protein degradation. By incorporating azobenzene photoswitches into PROTACs, photoswitchable versions named PHOTACs (PHOtochemically TArgeting Chimeras) were created. These molecules show little or no proteolytic activity in the dark, but can be activated with blue-violet light (380-440 nm). They can be used to degrade a variety of targets, including BRD2-4 and FKBP12, by binding to the CRL4^{CRBN} complex and promoting proteolysis in a light-dependent fashion. This translates to the optical control of protein levels and, in the case of BRD2-4, of cell proliferation, survival, and viability.

Design, synthesis, and photophysical characterization. The design of the PHOTACs was guided by a desire to render the molecules as diversifiable and modular as possible, whilst ensuring efficient synthetic access. To test the concept, CRBN was targeted which, together with VHL, accounts for the majority of PROTAC platforms utilized to date. Thalidomide derivatives, such as pomalidomide and lenalidomide, as CRBN ligands were utilized. As for the photoswitch, azobenzenes were used, which are known for their fatigue resistance, large and predictable geometrical changes, and easily tunable photothermal properties. Azobenzenes are also among the smallest photoswitches and do not significantly increase the molecular weight of pharmaceuticals upon substitution. PHOTACs should be inactive in the dark and lead to efficient degradation of the POI upon irradiation. Both regular azobenzenes (more stable in their trans form) and diazocines (more stable in cis) were explored.

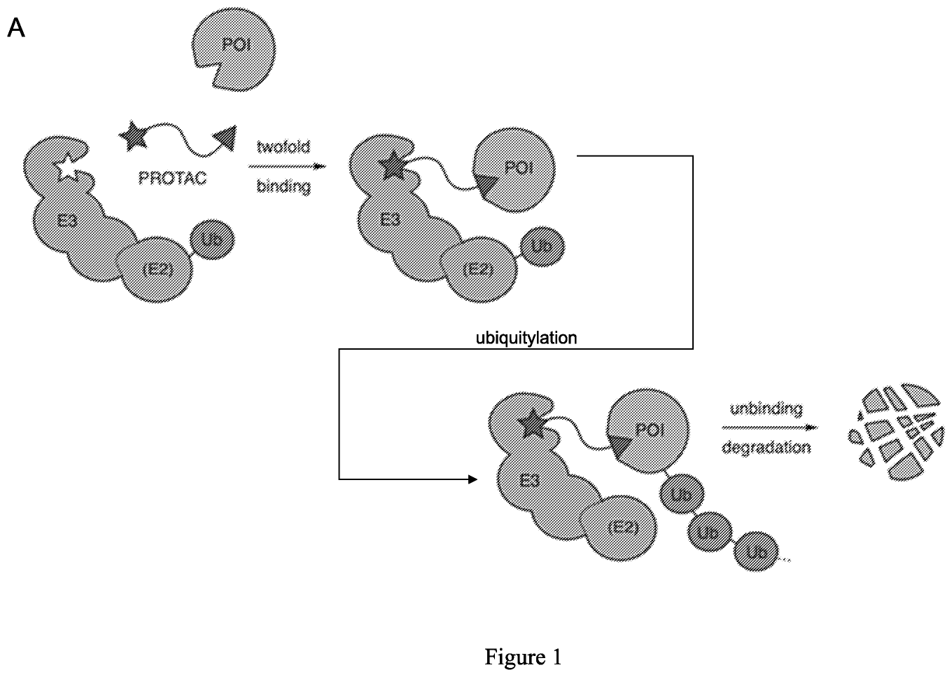

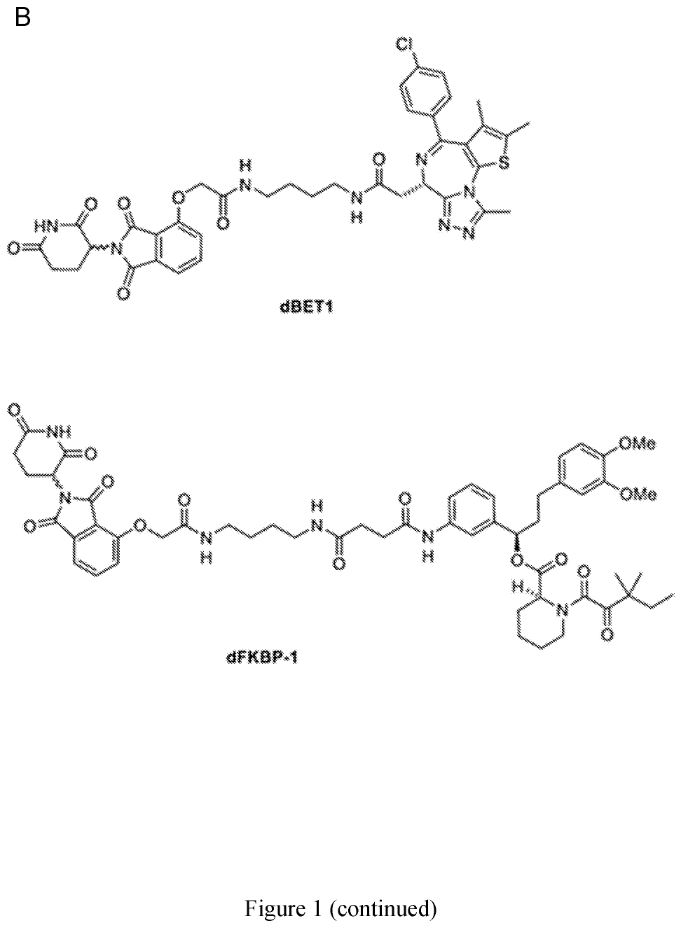

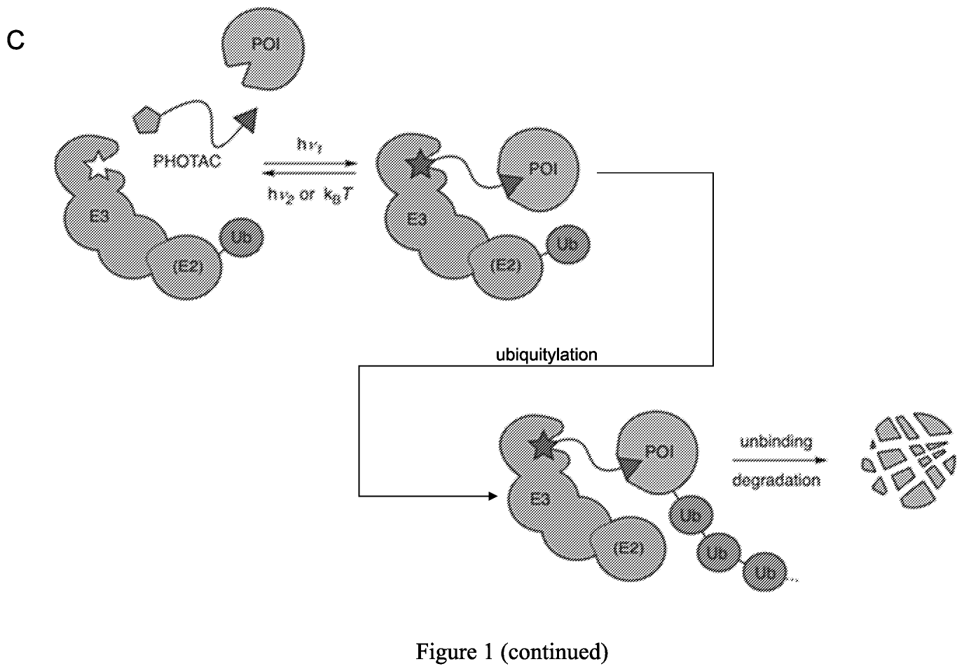

Several approaches are conceivable for the incorporation of these photoswitches into PHOTACs: (A) they could be part of the ligand for the E3 ligase and change the affinity at this end of the chimera; (B) the photoswitches could mostly reside in the tether, changing the length and orientation of this segment; (C) the azobenzenes could be part of a POI ligand, controlling the affinity of the PHOTAC to the POI. It would be difficult, however, to define a strict boundary between ligands and linker, and combinations of all three modes are possible. As for a first POI ligand, (+)-JQ1, a high-affinity inhibitor of BET proteins BRD2-4 and BRDT, was chosen. PROTACs featuring this ligand, such as dBET1 (FIG. 1B), have proven to be particularly effective and have been developed for a variety of E3 ligases.

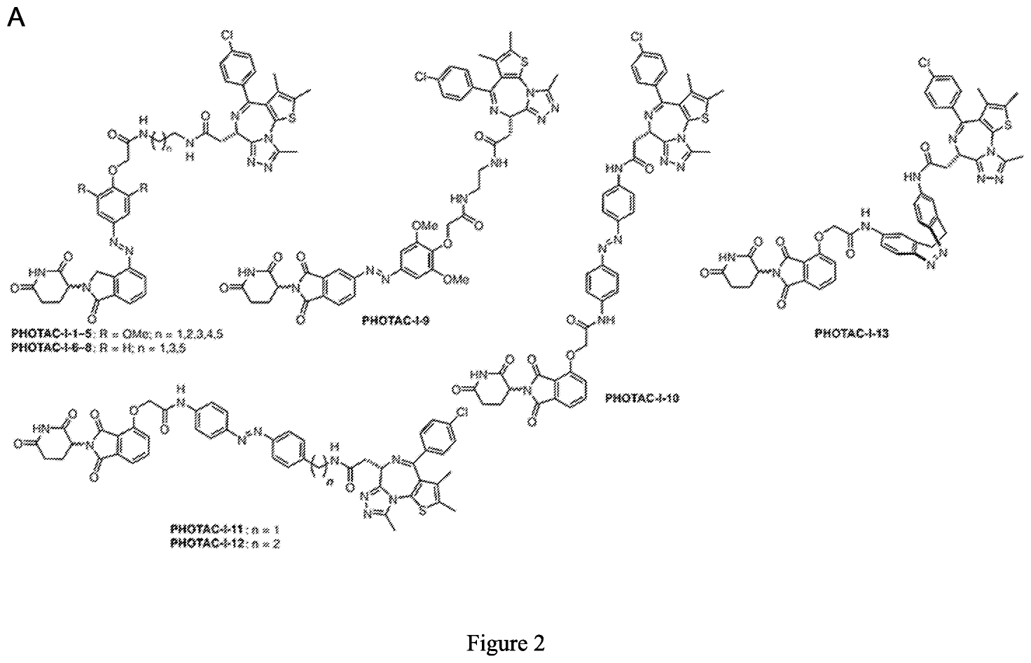

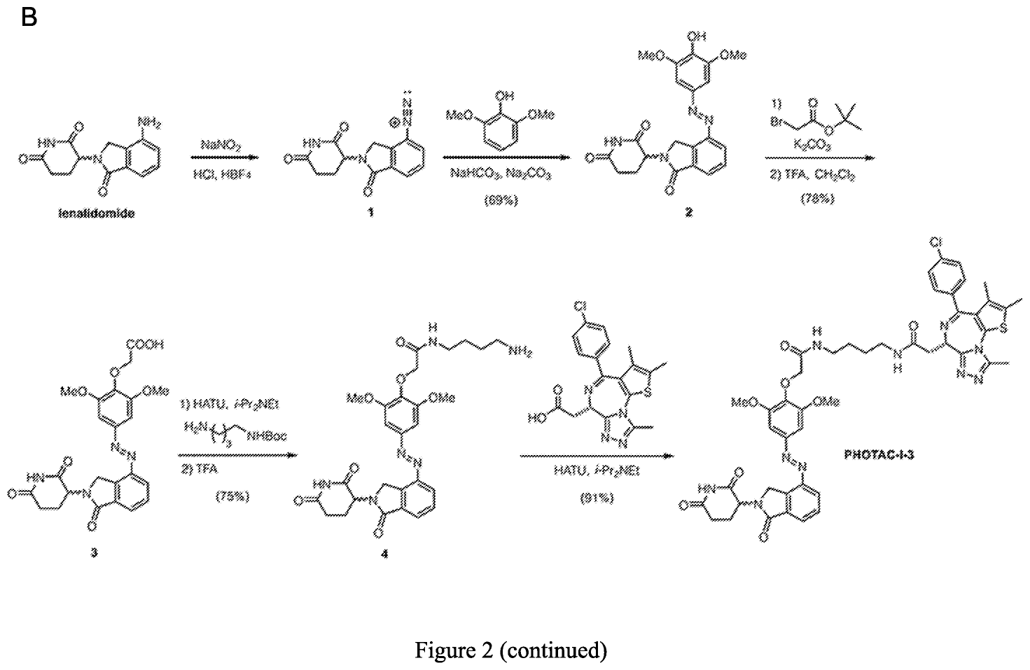

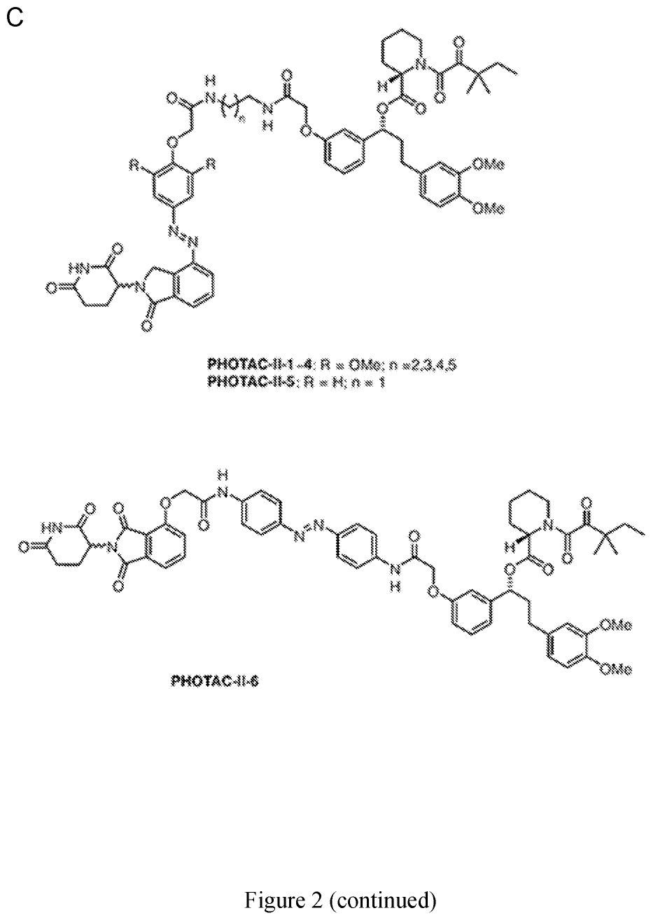

The small library of PHOTACs that resulted from these considerations is depicted in FIG. 2A. Amongst these, PHOTAC-I-3, emerged as one of the most effective. Its synthesis started with the diazotization of lenalidomide and coupling of the resulting diazonium ion 1 to 2,6-dimethoxyphenol, which yielded azobenzene 2 (FIG. 2B). Alkylation with tert-butyl bromoacetate and subsequent deprotection then afforded the key intermediate 3. Amide coupling of this carboxylic acid with N-Boc-butane-1,4-diamine and deprotection then yielded 4, which underwent another deprotection followed by peptide coupling with the free acid of (+)-JQ1 (5) to afford PHOTAC-I-3. HATU coupling of 3 to diaminoalkanes of different length provided easy access to library of PHOTACs with varying linker length (i.e. PHOTAC-I-1,2,4,5). PHOTACs-I-6-8, which lack two methoxy groups on the azobenzene core, and were synthesized analogously. PHOTAC-I-9 bears a different substitution pattern and was prepared via Baeyer-Mills coupling (see below). PHOTACs-I-10-13, which have the photoswitch more in the center of the molecule, were synthesized from 4-hydroxy thalidomide and azobenzene building blocks via alkylation and amide couplings (see below).

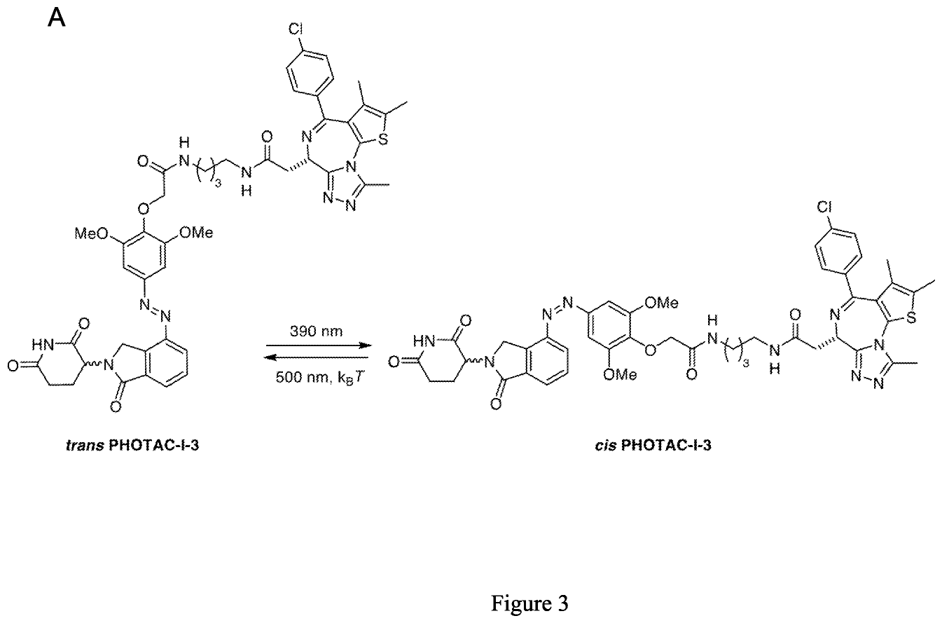

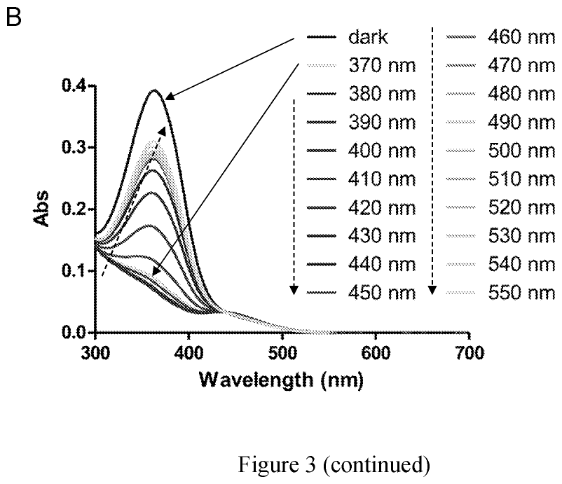

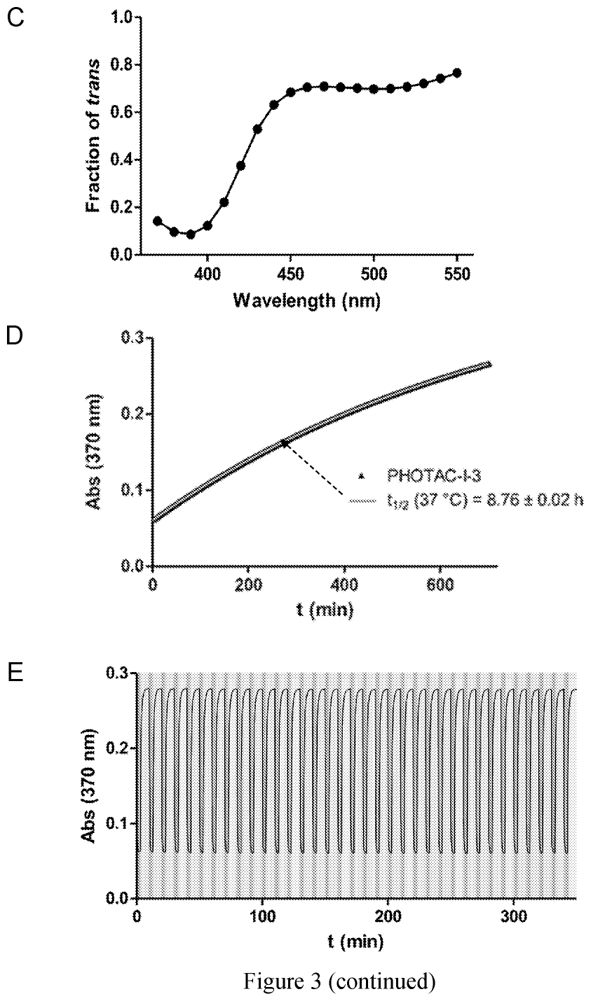

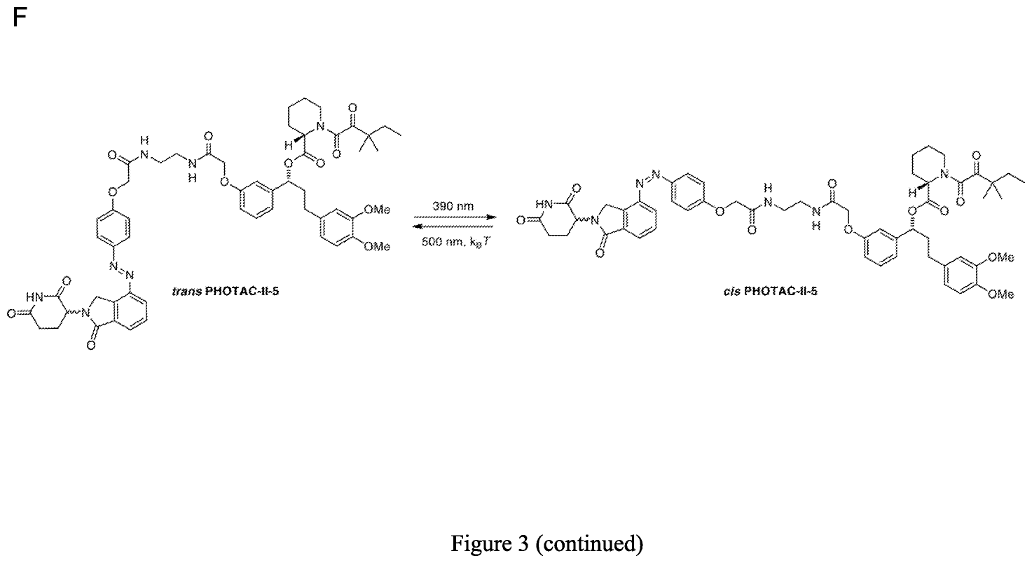

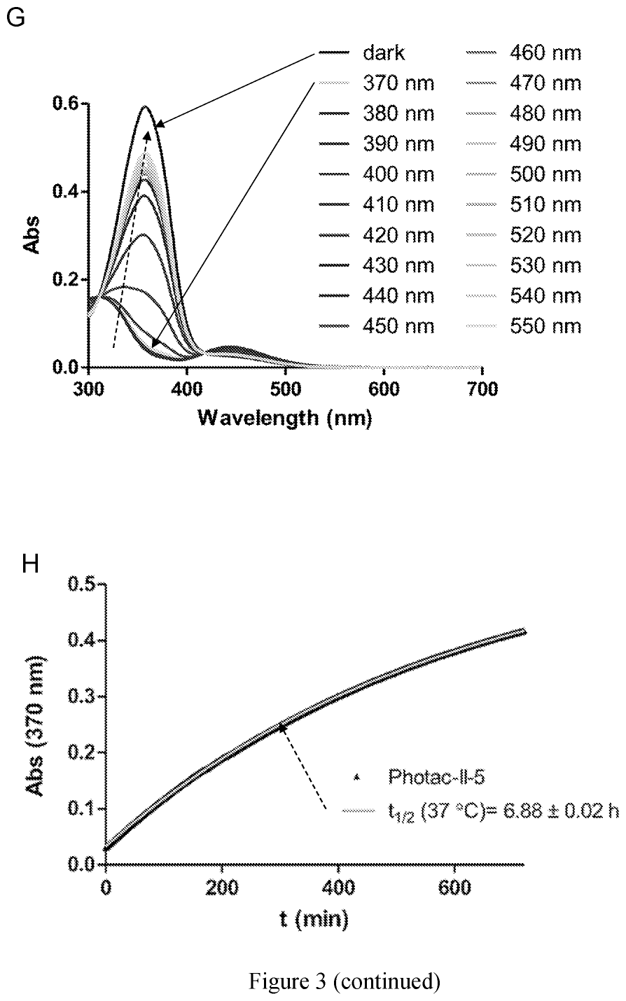

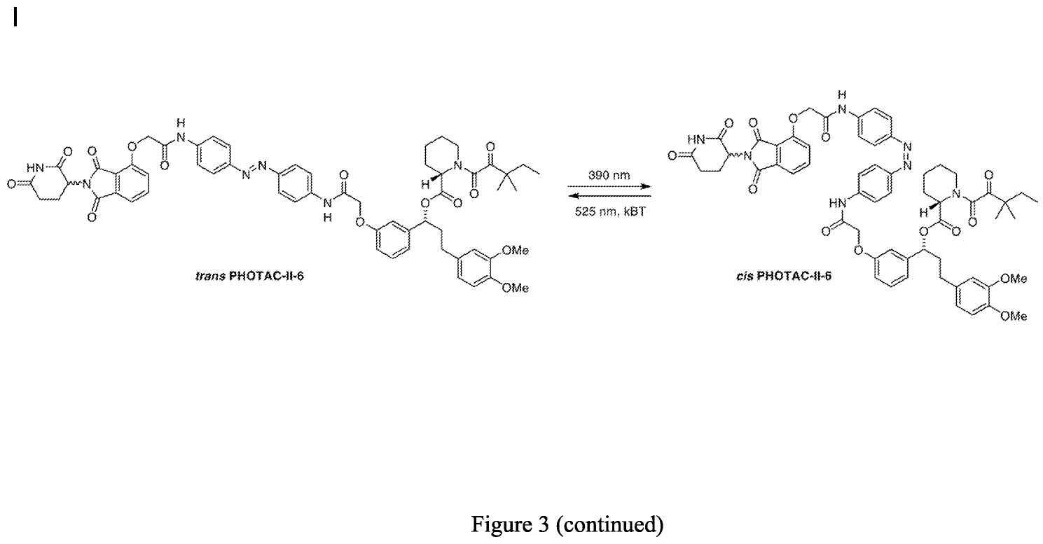

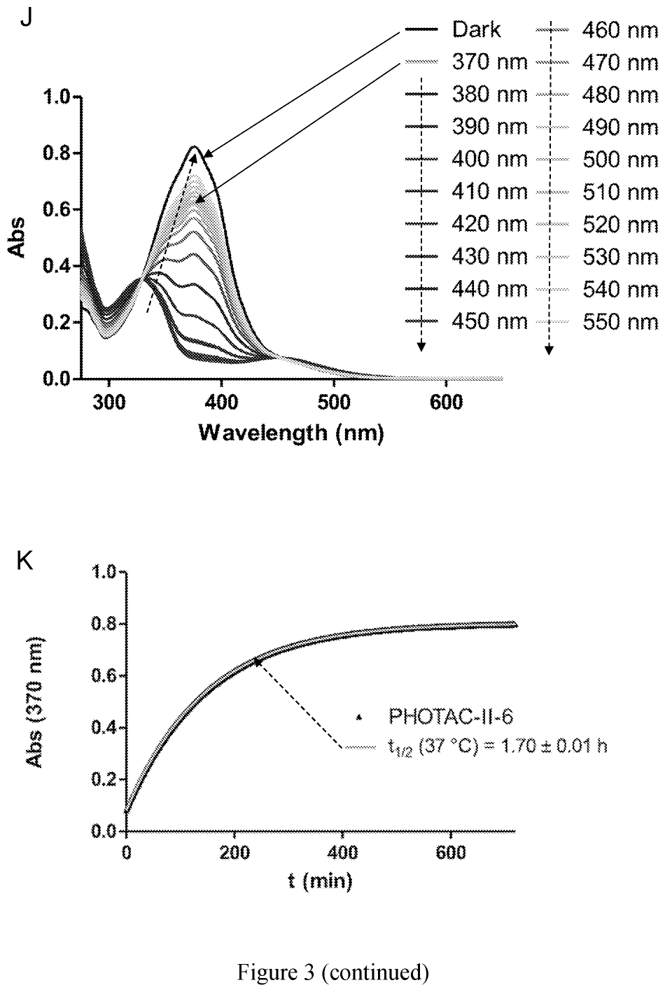

The photoswitching and thermal relaxation properties of one of the lead compounds, PHOTAC-I-3, is shown in FIG. 3A-E. The optimal wavelength to switch to the cis isomer is 390 nm but similar photostationary states (PSS) can be obtained between 380 and 400 nm (FIG. 3C). At 390 nm a PSS of >90% cis could be obtained. Rapid cis to trans isomerization could be achieved by irradiation with wavelengths >450 nm, achieving PSS of ca >70% trans (FIG. 3C). In the absence of light, cis PHOTAC-I-3 slowly isomerized back to its trans form with a half-life of 8.8 h at 37° C. in DMSO (FIG. 3D). Multiple cycles of photochemical isomerization are possible, in keeping with the fatigue-resistance of azobenzene photoswitches (FIG. 3E). Structurally related PHOTACS showed similar photophysical and thermal properties (see below).

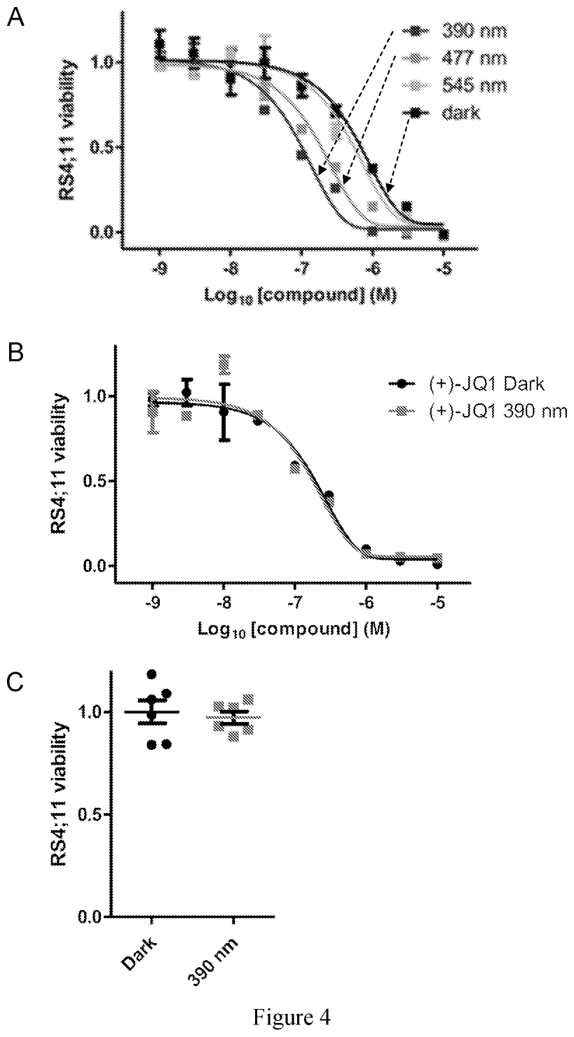

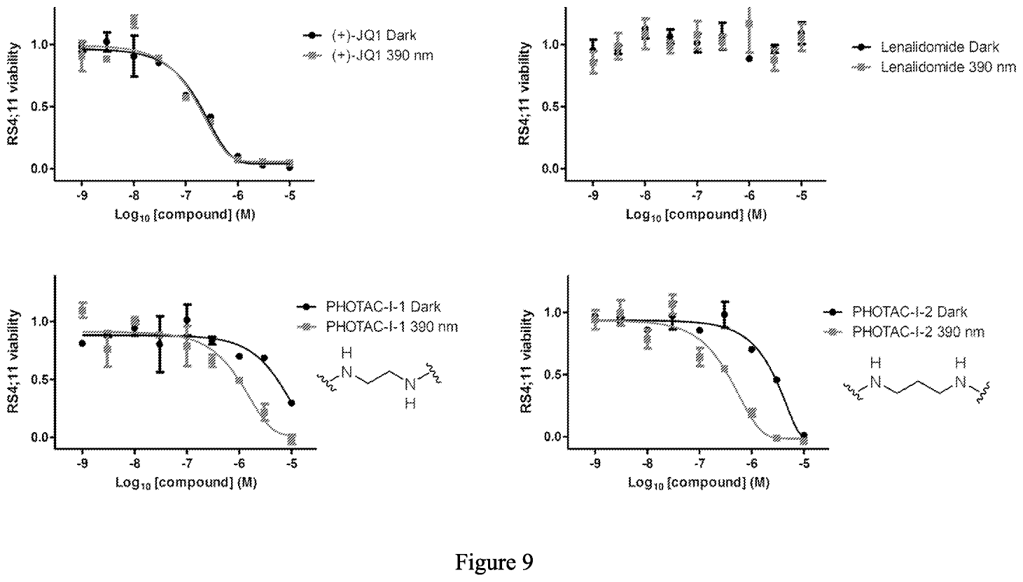

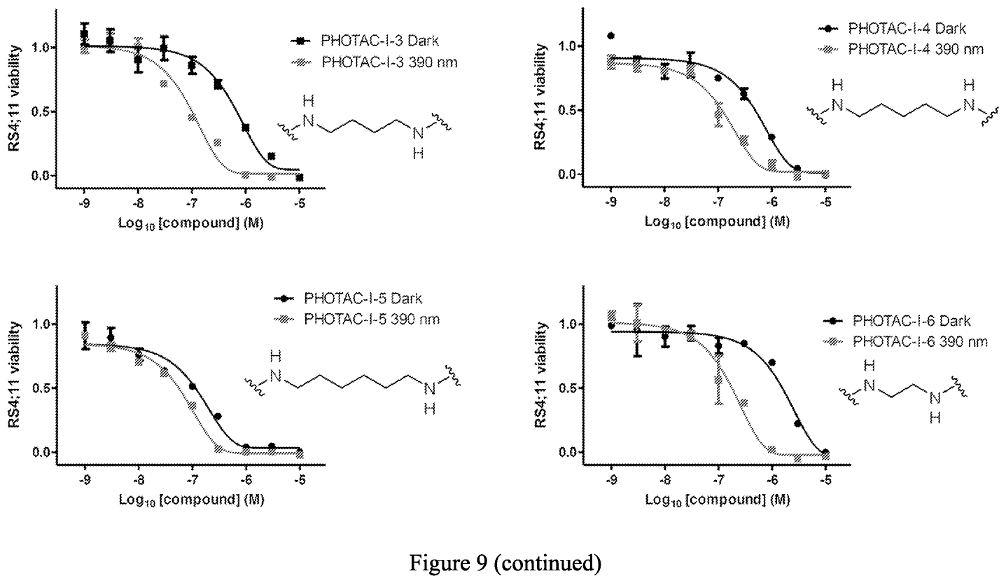

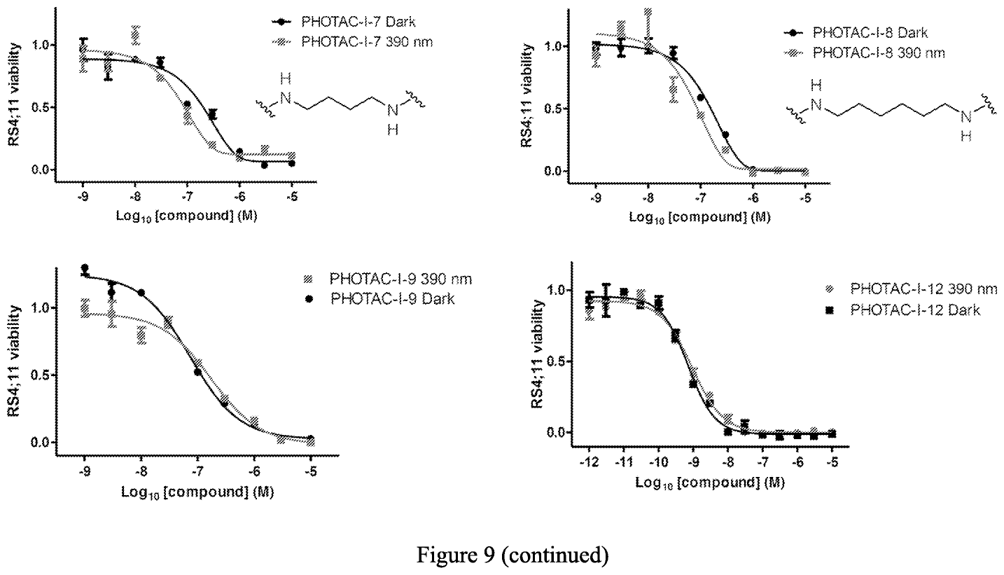

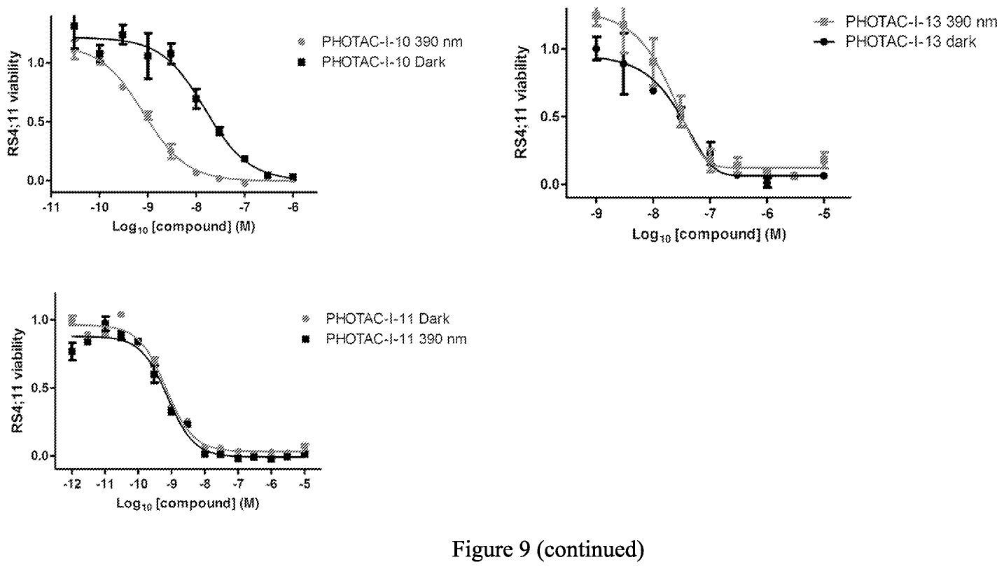

Optical control of BRD2-4 with PHOTACs. To assess the biological activity of the PHOTACs, their effect on the viability of RS4;11 lymphoblast cells was tested. Cells were treated in a 96-well plate with increasing concentrations of PHOTACs and were either irradiated with 390 nm light pulses (100 ms every 10 s) for 72 h or incubated with the compound in the dark. Subsequently, cell viability assays (Promega MTS) were performed, as previously described. PHOTAC-I-3 showed a promising activity difference upon irradiation (FIG. 4A). The EC₅₀ was determined to be 88.5 nM when irradiated with 390 nm light and 631 nM in the dark, resulting in a 7.1-fold EC₅₀ difference. This indicates that cytotoxicity increases upon irradiation and that PHOTAC-I-3 is less toxic in the dark. Similar trends were observed for PHOTAC-I-1,2,4-8,10, all of which were more active in viability assays following pulse irradiation (FIG. 9). By contrast, PHOTACs-I-9, 11-13 showed no light-dependent differences in activity (FIG. 9). In a control experiment, the BET inhibitor (+)-JQ1 alone showed no light-dependent toxicity either (FIG. 4B).

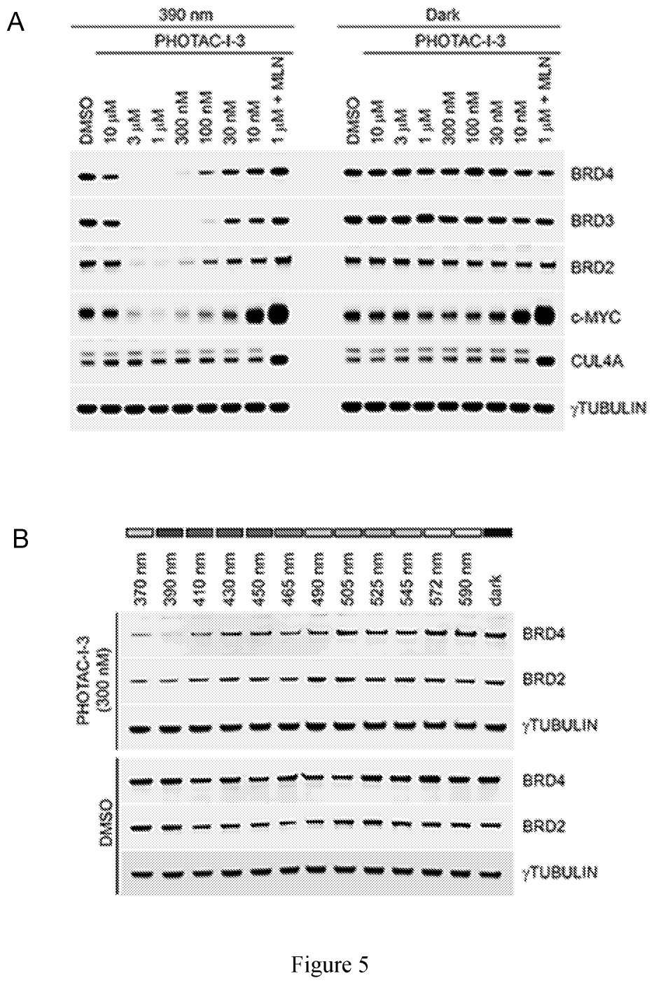

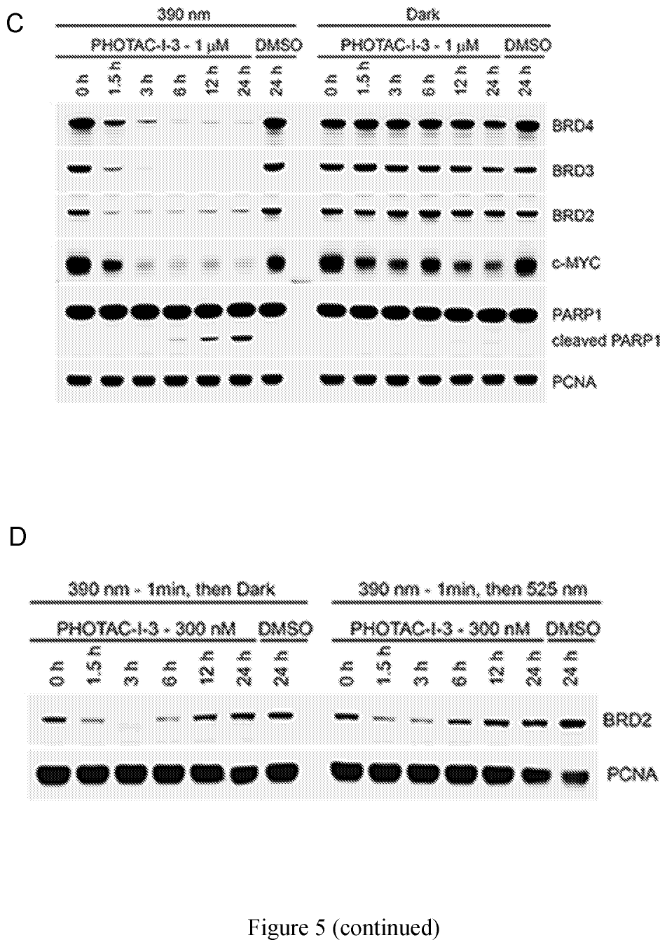

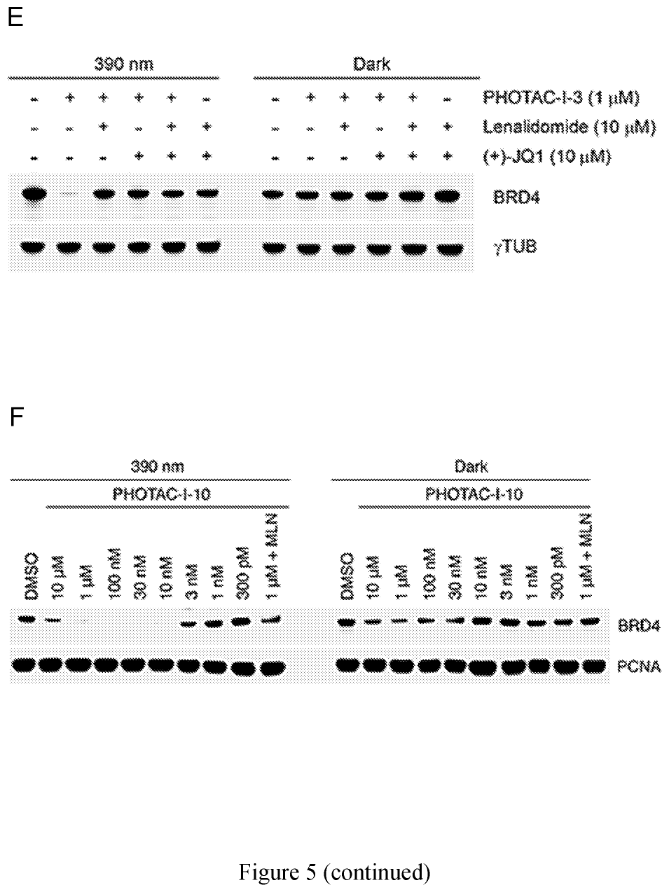

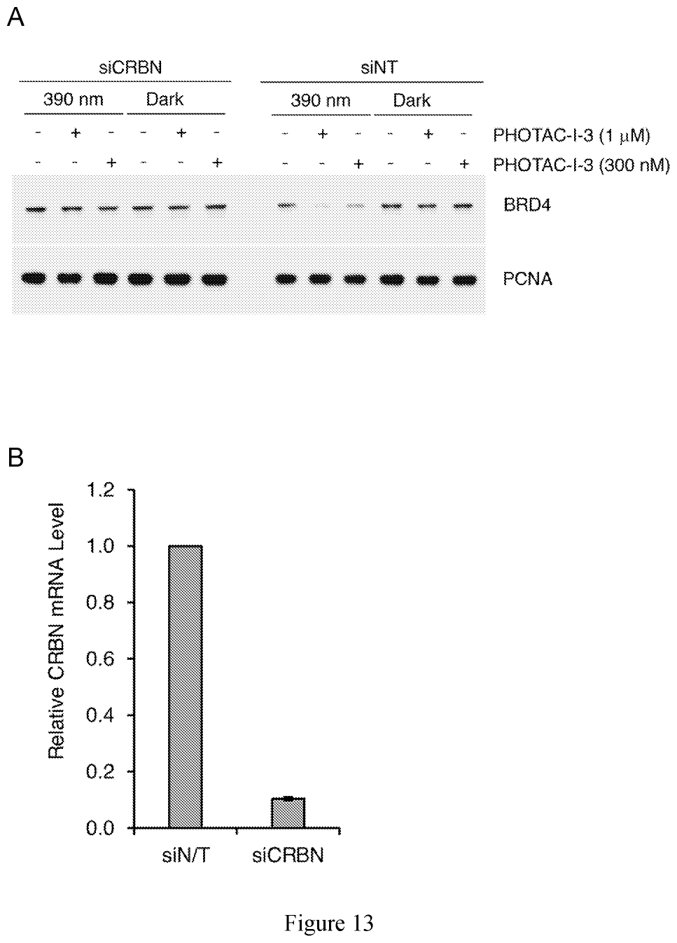

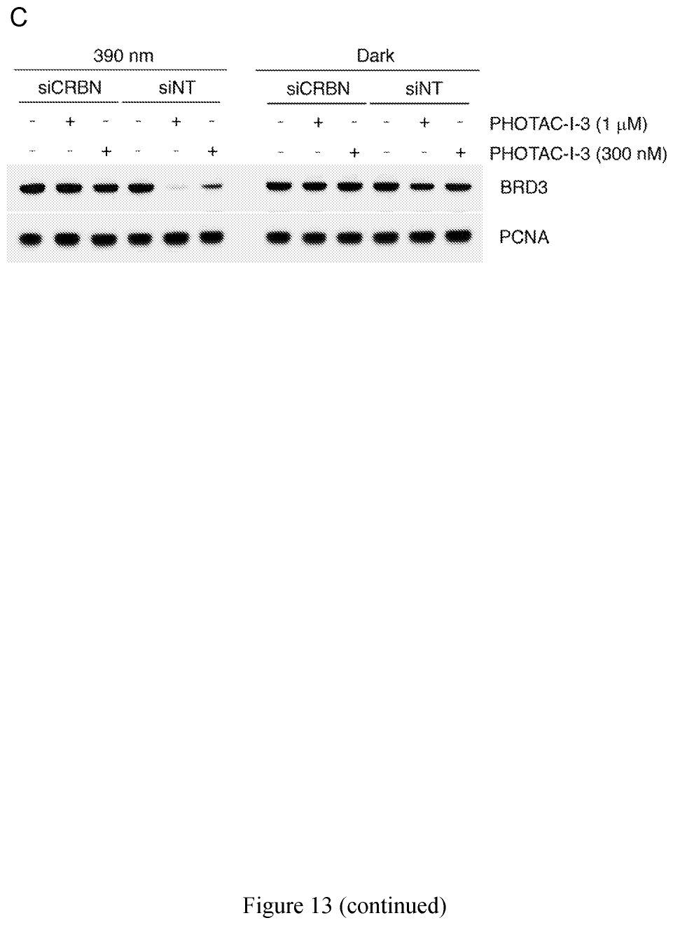

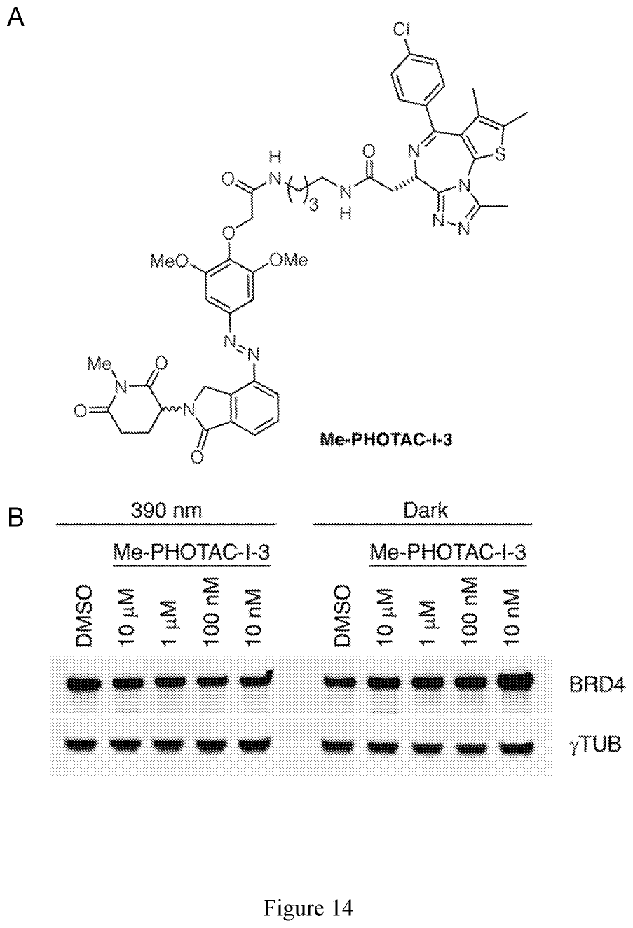

Next, the light dependence of targeted protein degradation was analyzed in RS4;11 cells by western blot analysis of the BET proteins BRD2-4 (FIG. 5). To this end, cells were treated with increasing concentrations of our lead compound, PHOTAC-I-3, for 4 h and pulse-irradiated with 390 nm light (100 ms every 10 s). A pronounced decrease of BRD4 levels was observed in the presence of PHOTAC-I-3 (particularly between 100 nM to 3 μM) when irradiated with 390 nm light, but not in the dark (FIG. 5A). At 10 μM, less degradation was observed, which is consistent with the “hook effect” commonly observed with PROTACs. BRD3 levels were also significantly reduced upon exposure to concentrations in the range of 100 nM to 3 μM of PHOTAC-I-3 when irradiated with violet light, but not in the dark. In comparison, BRD2 was degraded to a lesser extent and within a narrower concentration range. Application of PHOTAC-I-3 (1 μM) together with the CRL inhibitor MLN4924 (2.5 μM), which inhibits neddylation and, consequently, the activity of all cellular CRLs (including CRL4^{CRBN}), rescued BRD2-4 levels upon irradiation. The cereblon-dependent degradation was confirmed by a competition experiment (FIG. 5E), and further validated by siRNA knockdown of cereblon (FIG. 13). Methylation of the glutarimide prevented the degradation of BRD4 as previously demonstrated (FIG. 14).

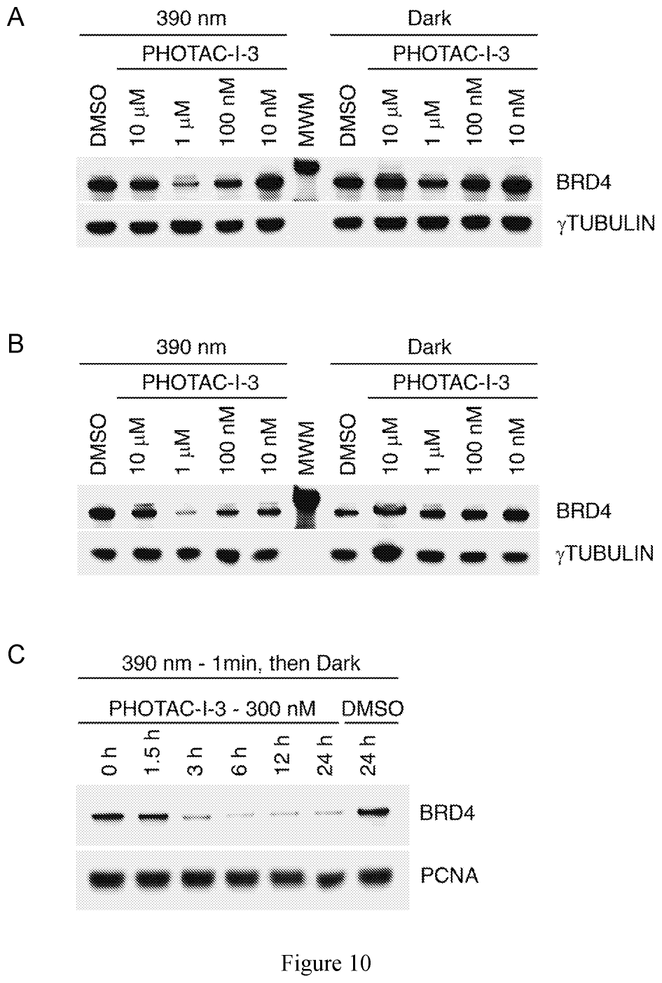

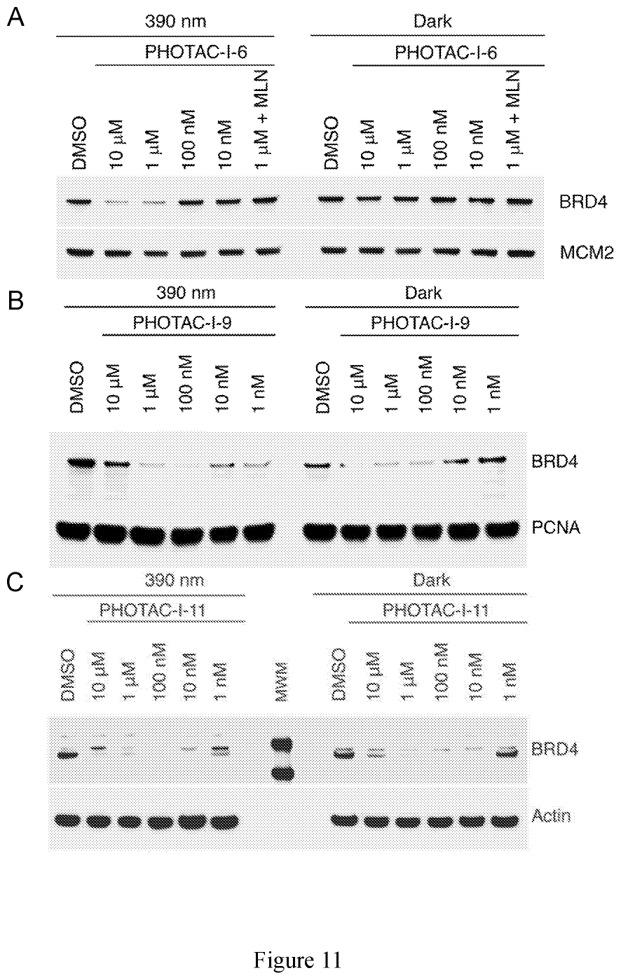

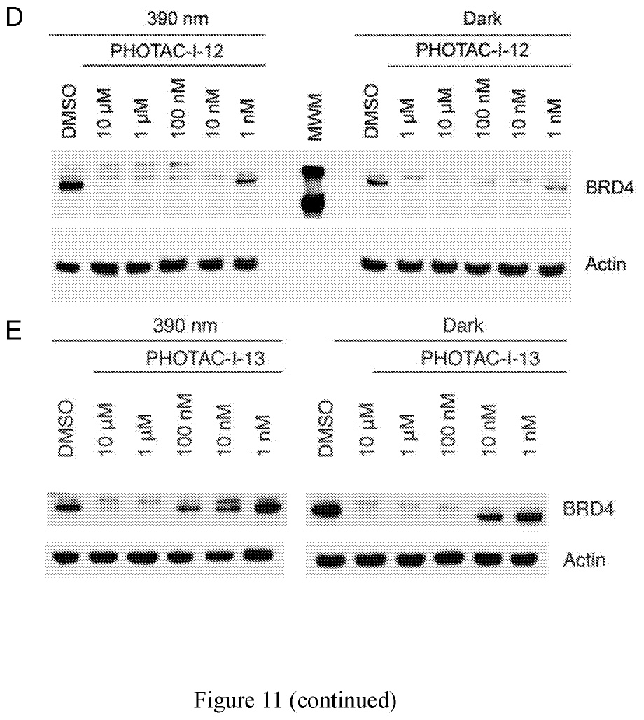

Photoactivatable degraders and inhibitors of BRD4, c-MYC levels were also affected. Downregulation of this transcription factor, which is a notoriously difficult target for pharmacological intervention, was more pronounced when cells were pulse-irradiated in the presence of low concentrations of PHOTAC-I-3 than in the dark. The light-dependent degradation of BRD4 by PHOTAC-I-3 was also confirmed in two breast cancer cell lines (MB-MDA-231 and MB-MDA-468) (FIG. 10A,B). Immunoblot analyses of additional PHOTACs are presented in FIG. 11.

The time dependence of BRD degradation is shown in FIG. 5B. BRD2 and BRD3 are largely absent after 1.5 h exposure and irradiation, whereas BRD4 is degraded more slowly. Sustained degradation and c-MYC downregulation over 24 hours was also observed. In the dark, PHOTAC-I-3 had no effect on BRD levels and relatively little effect on c-MYC levels. The slight effect on c-MYC can be explained by inhibition of BRD4 with the (+)-JQ1 derivative PHOTAC-I-3 in the absence of targeted degradation. Following sustained pulse irradiation, increasing cleavage of PARP-1 was also observed (FIG. 5B). This indicator of apoptosis correlates to the cell viability assay shown in FIG. 4A. Persistent degradation of BRD4 in the dark could be achieved following a brief activation of PHOTAC-I-3 for 1 min (FIG. 10C).

One of the principal advantages of photoswitches over caged compounds is their reversibility. Azobenzene photoswitches can thermally relax to an inactive form or be isomerized back photochemically. The photochemical reversibility was demonstrated with a rescue experiment wherein PHOTAC-I-3 was continuously irradiated for 1 min with the activating wavelength (390 nm) and then pulse irradiated with the deactivating wavelength (525 nm). Under these conditions, cellular BRD2 levels initially decreased but, subsequently, recovered faster than when left in the dark (FIG. 5C).

Another characteristic feature of photopharmacology is “color-dosing” (i.e., the ability to control the concentration of the active species with the color of the incident light). The photostationary state (i.e., the ratio between the two isomers) is a function of the wavelength. FIGS. 3A and 5D show that this principle can also be applied to PHOTACs. Cell viability assays gave left-shifted curves as the color gradually approached 390 nm (FIG. 4A). Western blots showed maximum degradation at 390 nm and gradually increasing BRD4 levels as the wavelength of the incident light increased (FIG. 5D). At 370 nm slightly increased protein levels were observed, in agreement with the photostationary states measured at different wavelengths, which are maximized toward the active cis isomer at the slightly longer wavelength of 390 nm (FIG. 3C).

The effect of PHOTAC-I-10, which is derived from thalidomide and has the photoswitch positioned deeper in the linker, on BRD4 levels, is shown in FIG. 5E. Robust photodegradation was found even with 10 nM PHOTAC-I-10. A clear hook-effect was observed. Once again, the thermally less stable cis azobenzene promoted ubiquitylation and degradation.

Optical control of FKBP12 with PHOTACs. To test whether the PHOTAC approach is generalizable, the prolyl cis-trans isomerase FKBP12 was utilized. The structures of the corresponding PHOTACs are shown in FIG. 2C. PHOTACs of this series consist of a CRBN-targeting glutarimide, an azobenzene photoswitch in different positions, a linker, and the ligand SLF that binds to native FKBP12. The synthesis of PHOTACs-II-1-6 is detailed herein. The photophysical and thermal characterization of PHOTACs-II-5 and PHOTACs-II-6 is shown in FIG. 3 F-H and in FIG. 3 I-K, respectively.

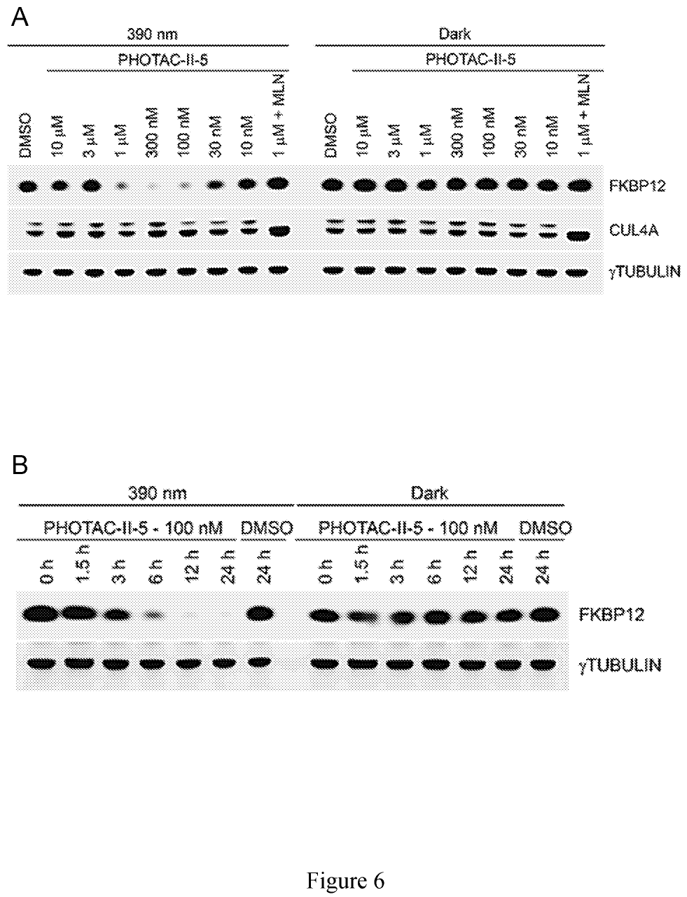

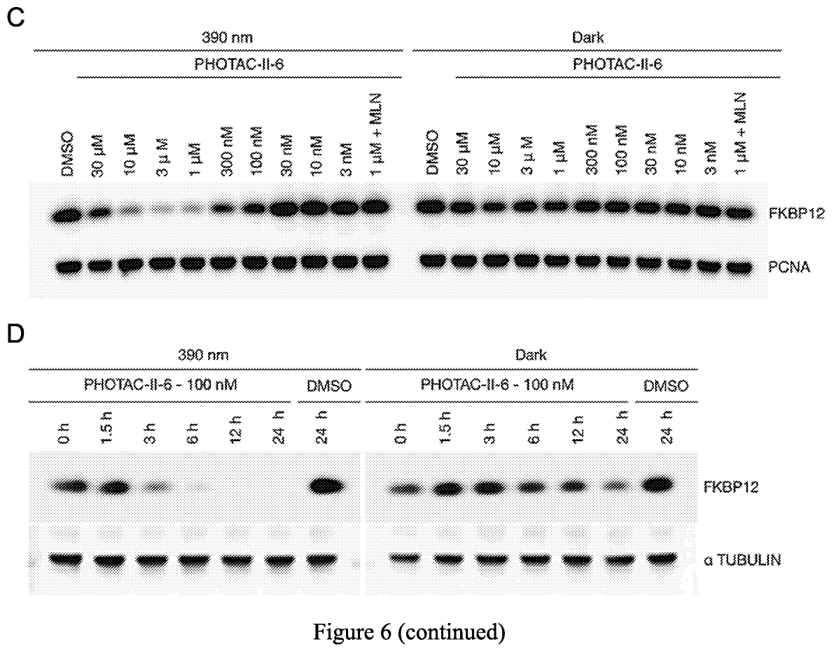

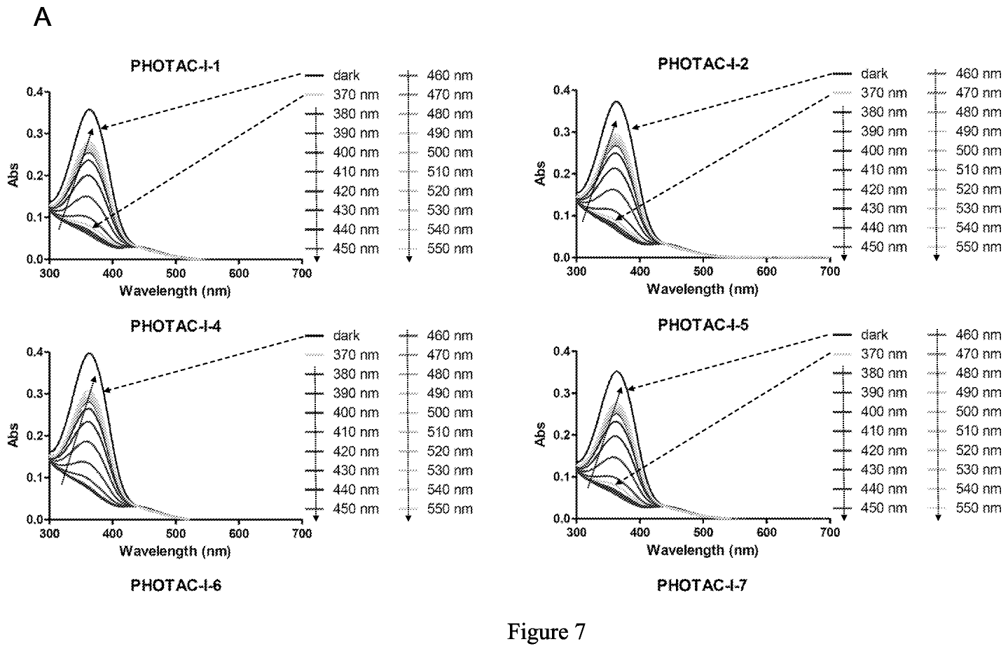

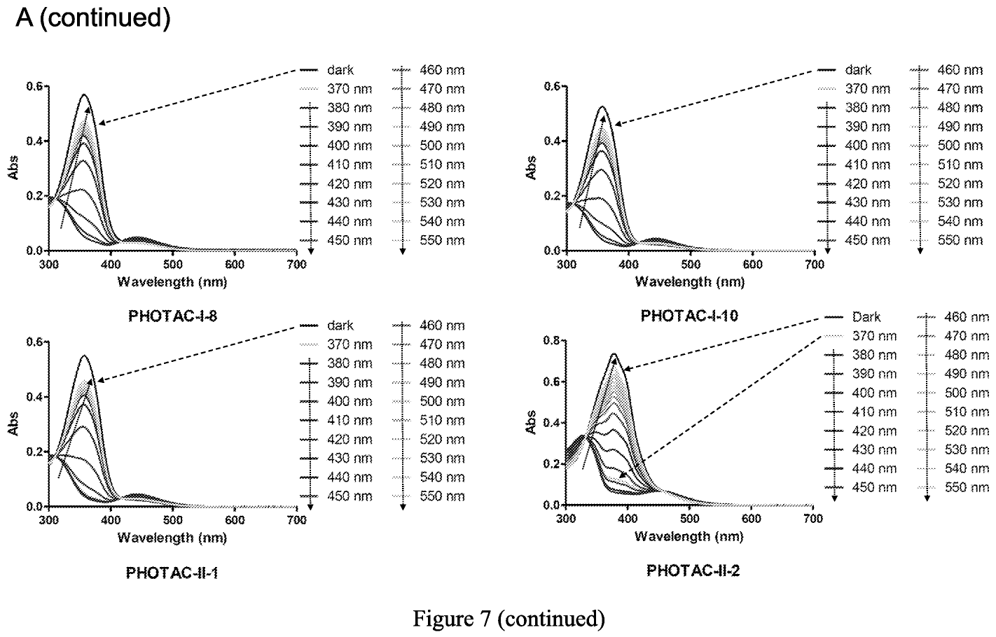

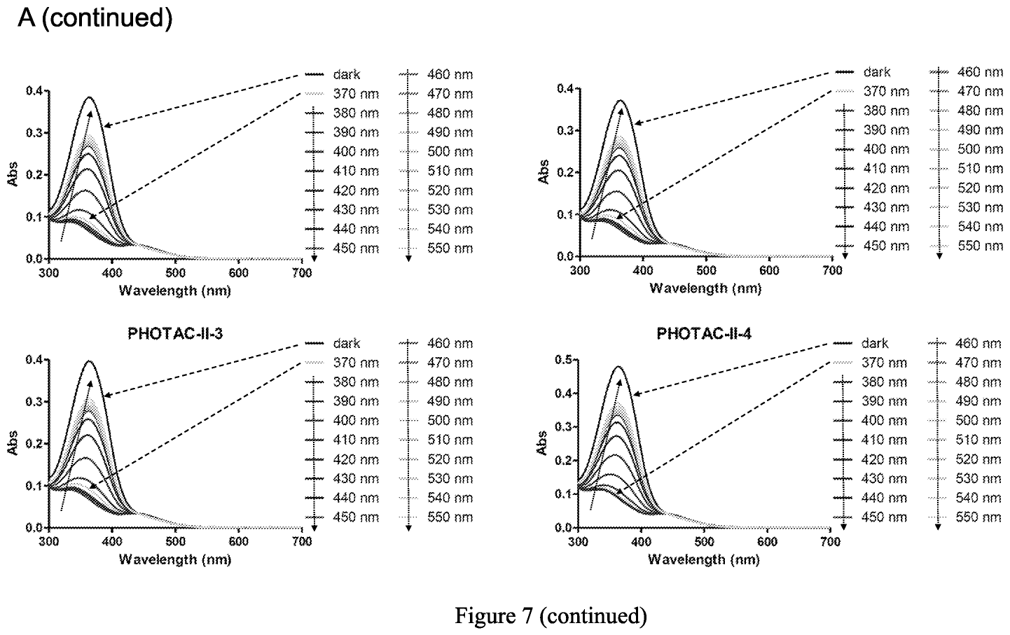

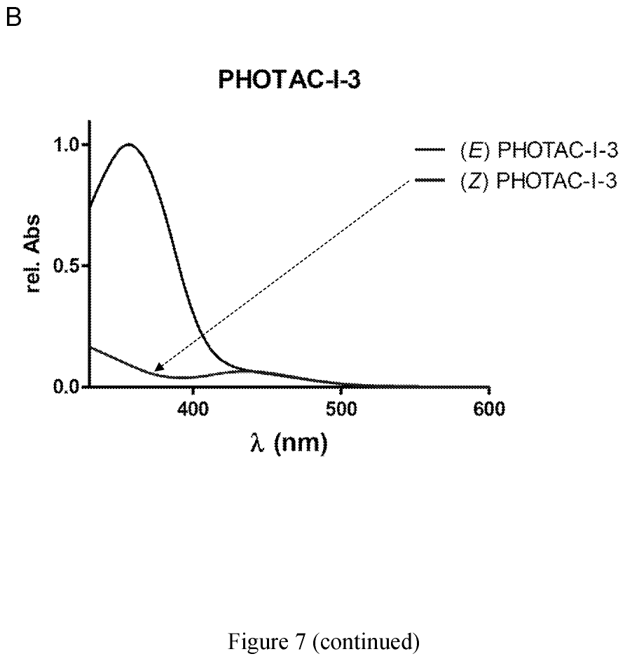

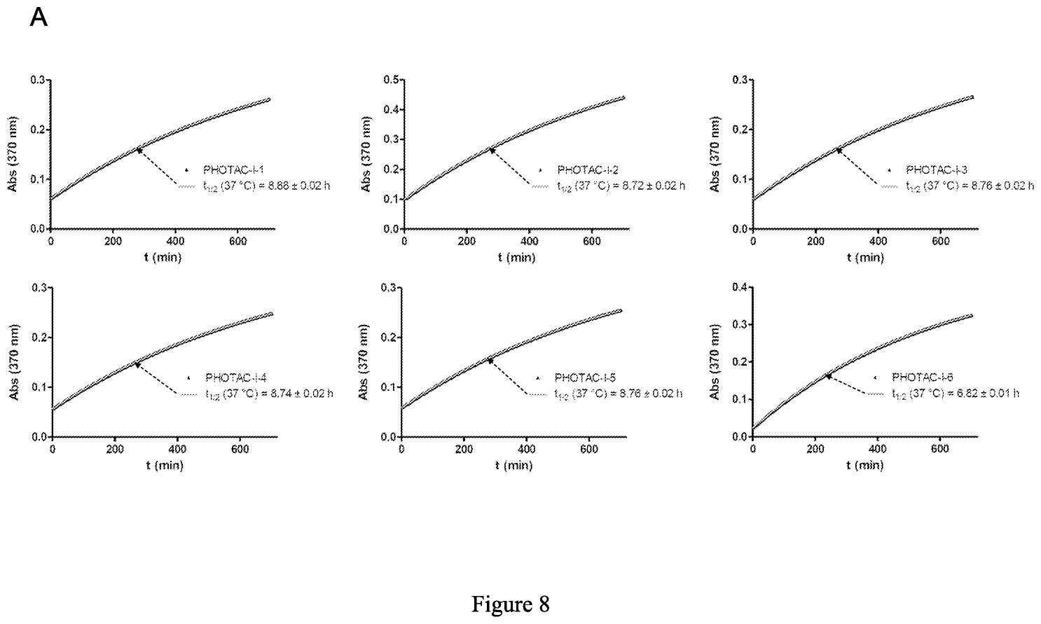

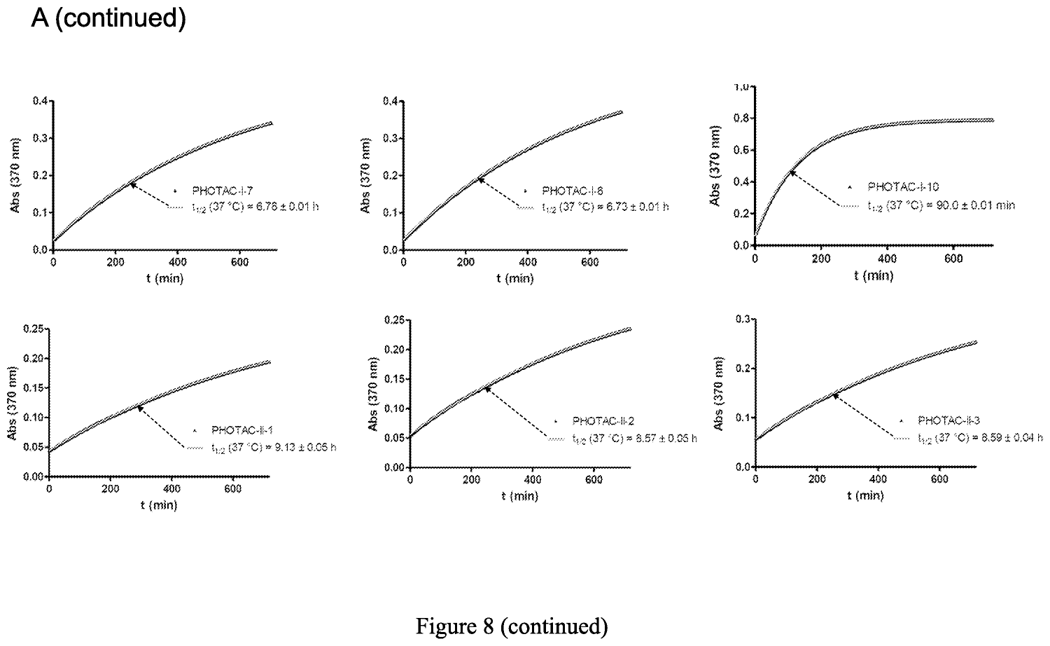

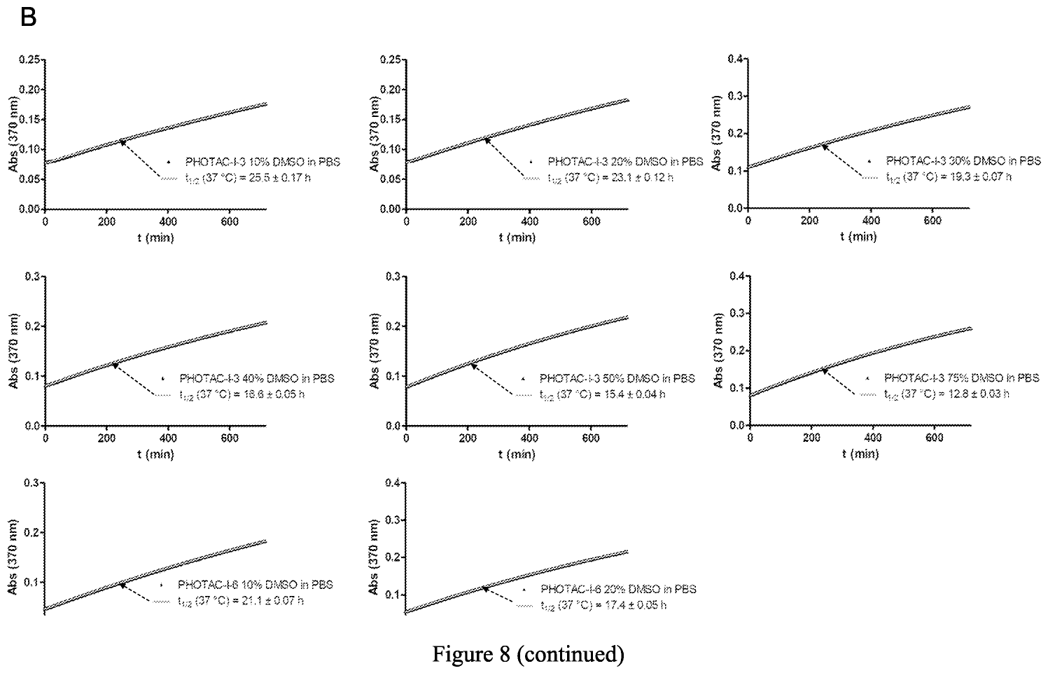

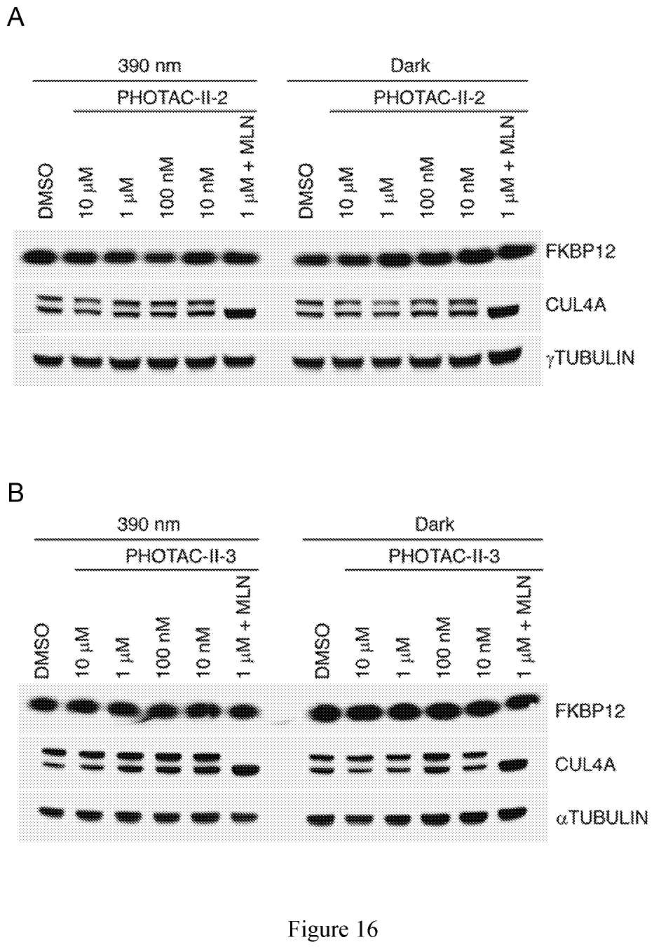

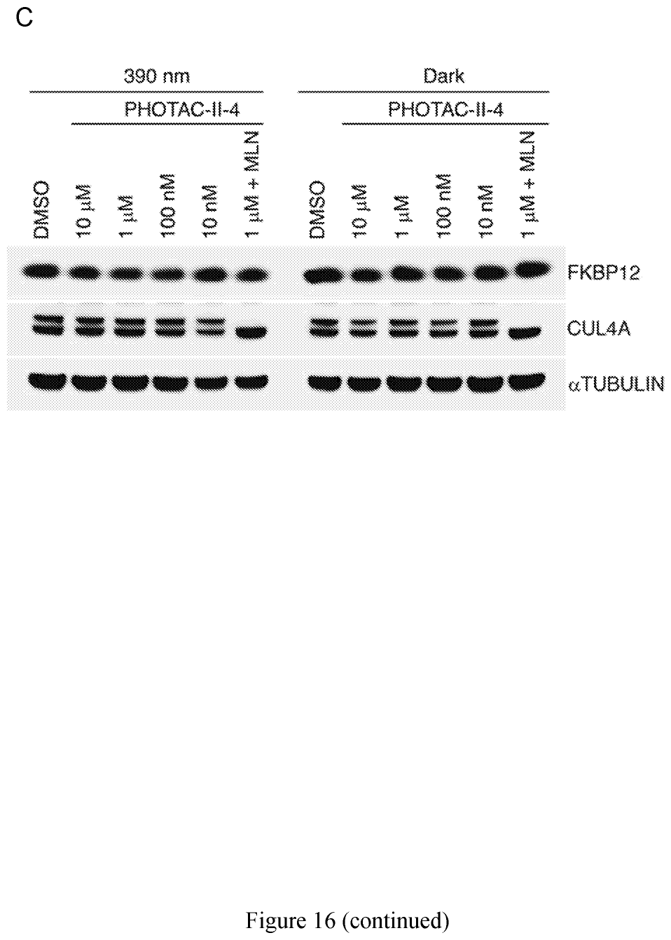

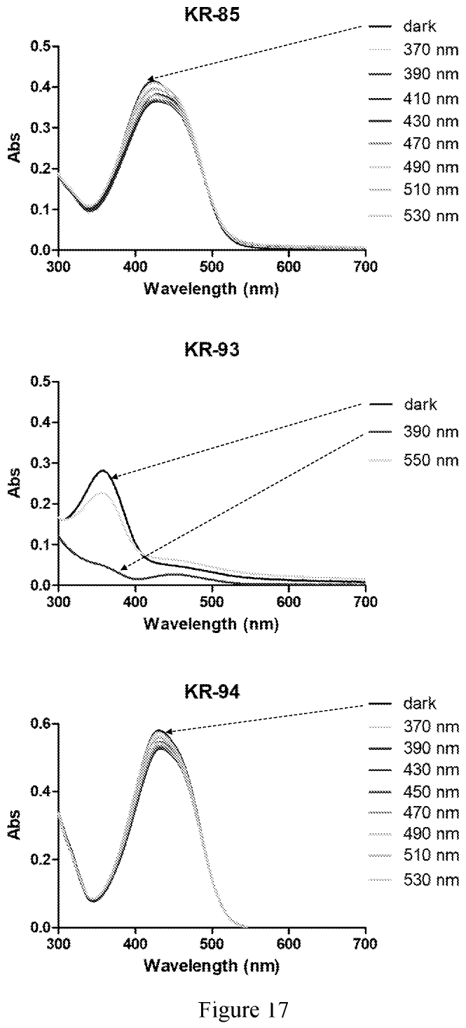

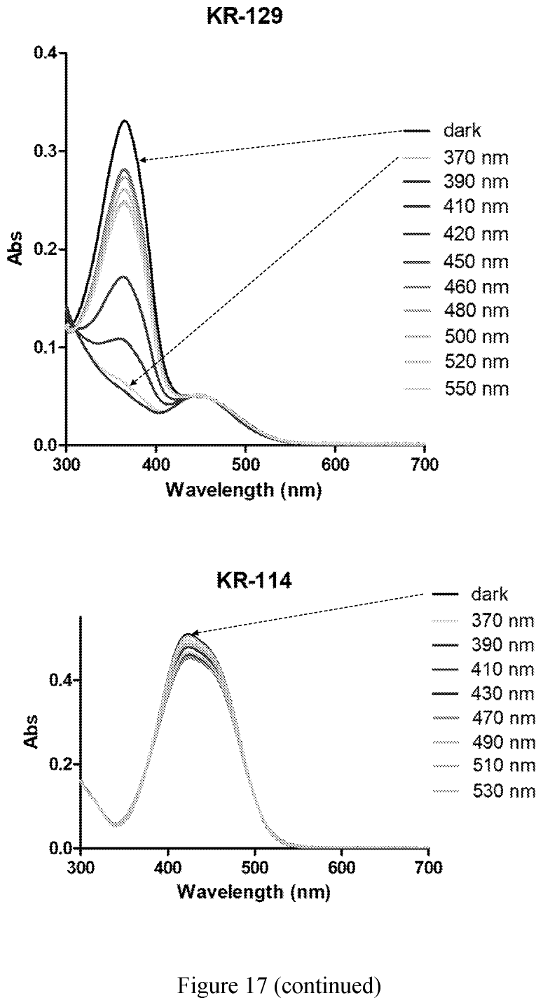

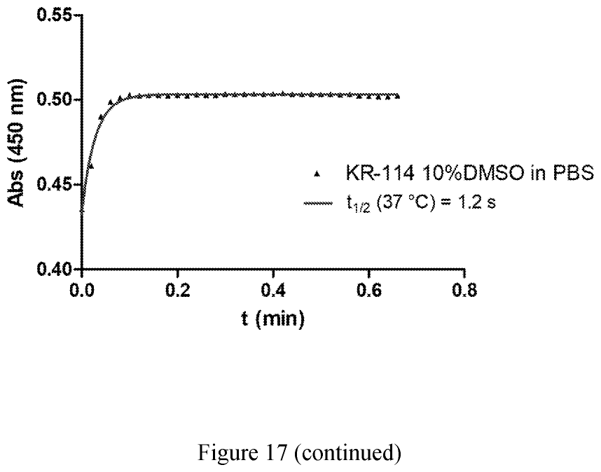

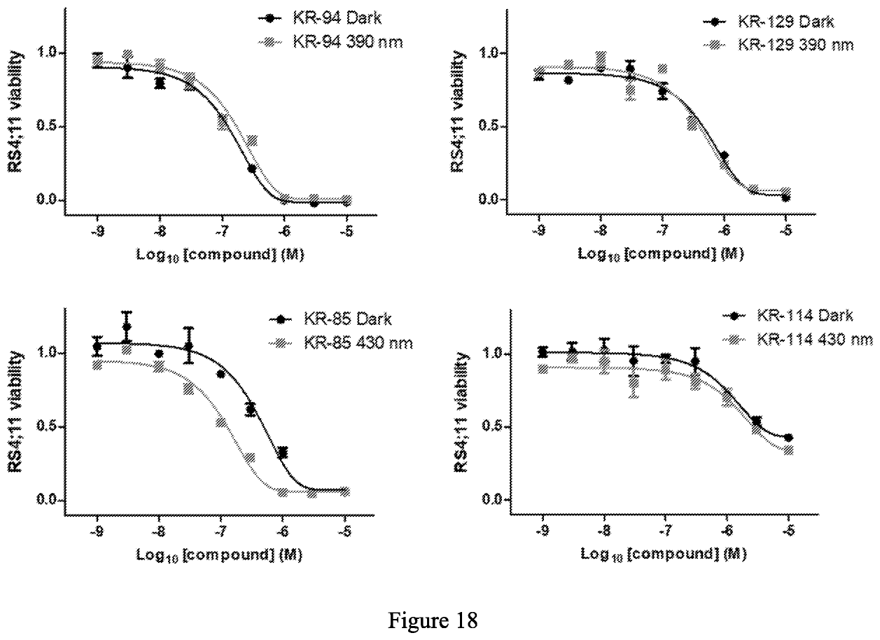

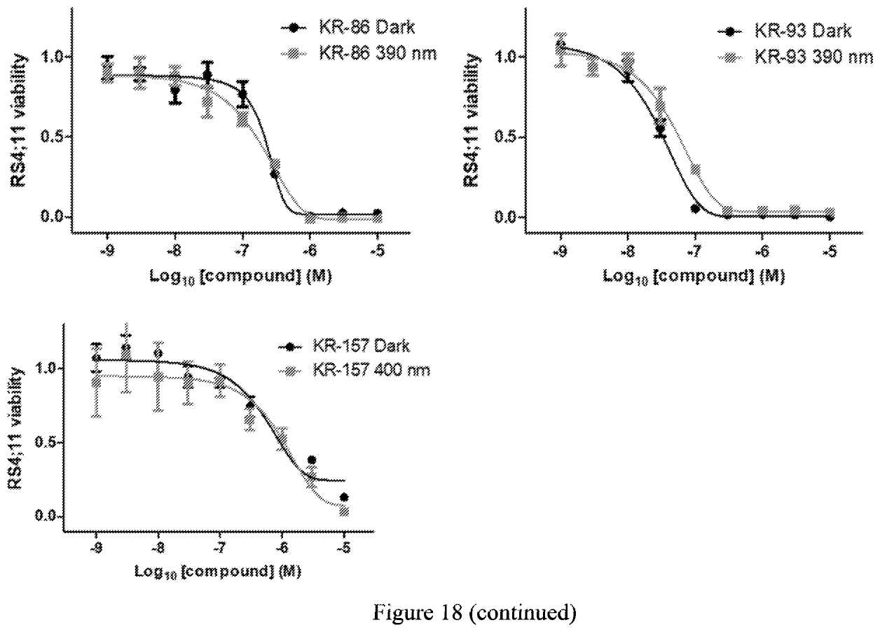

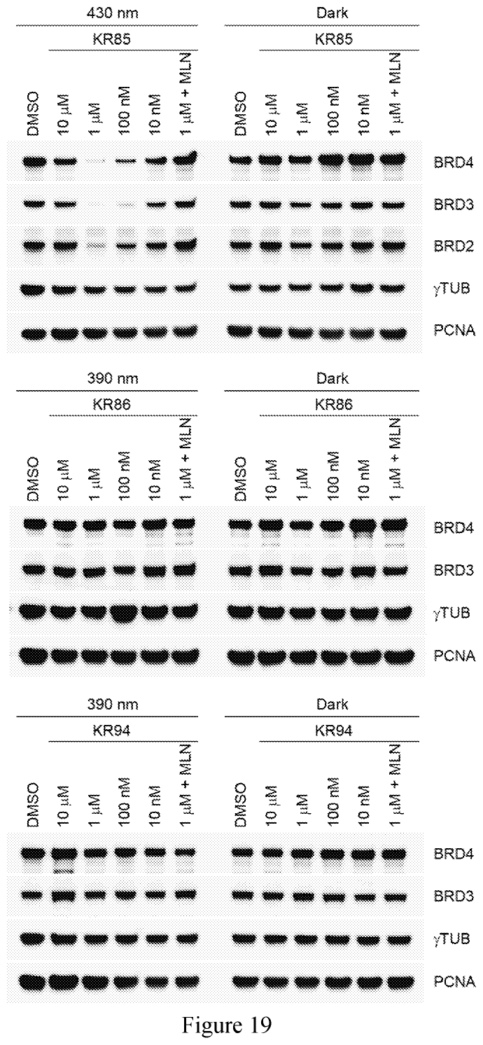

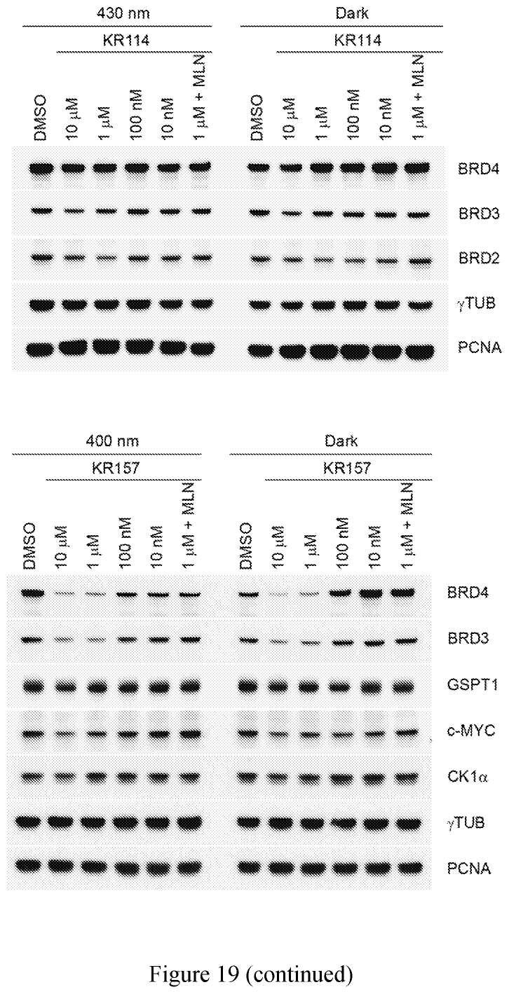

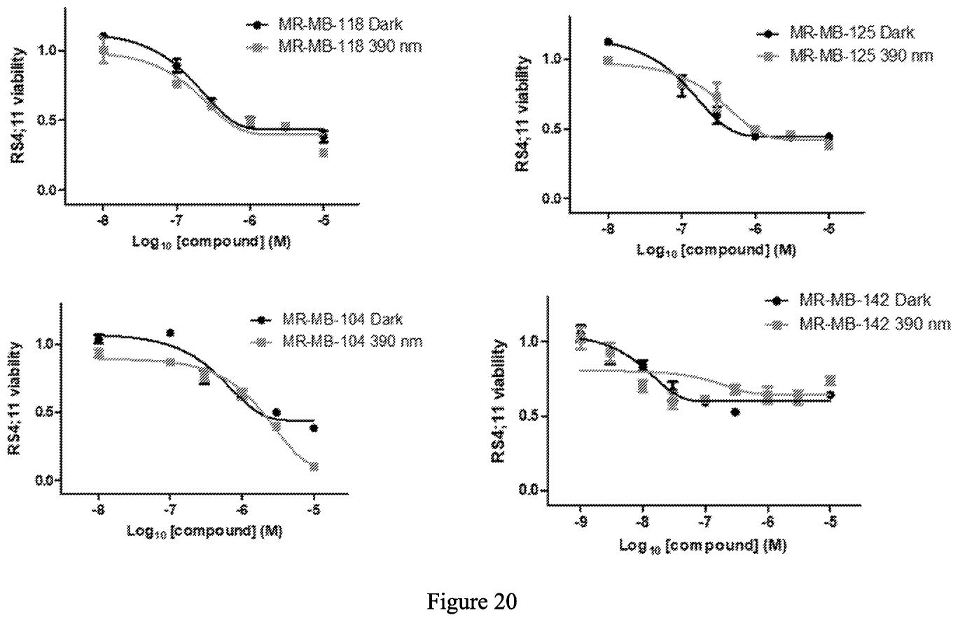

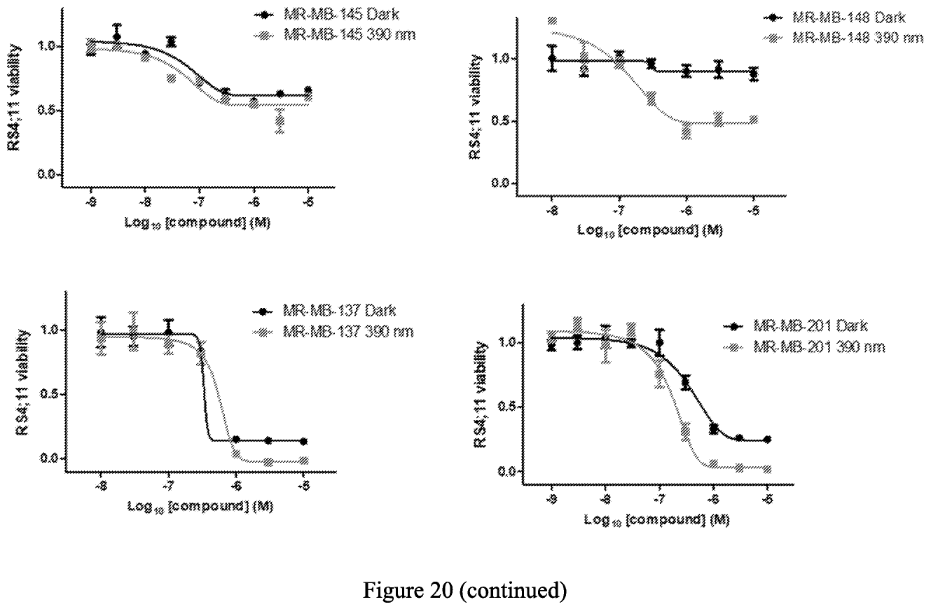

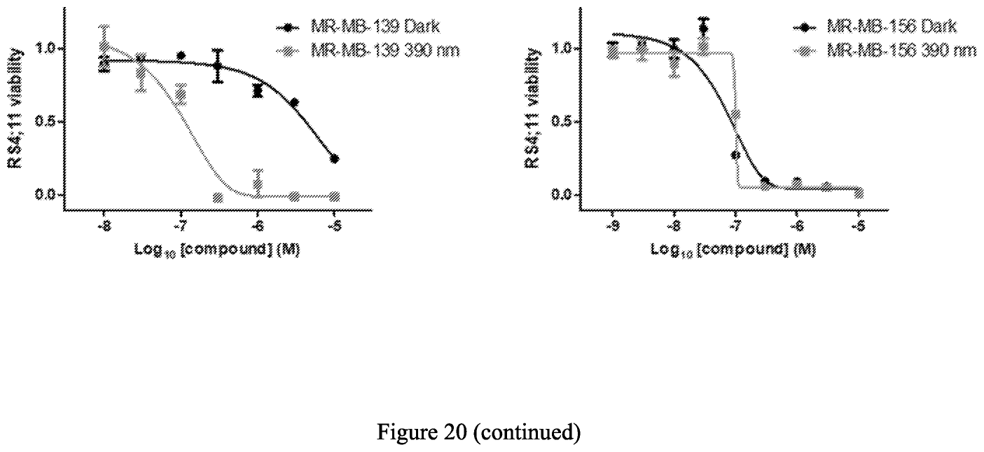

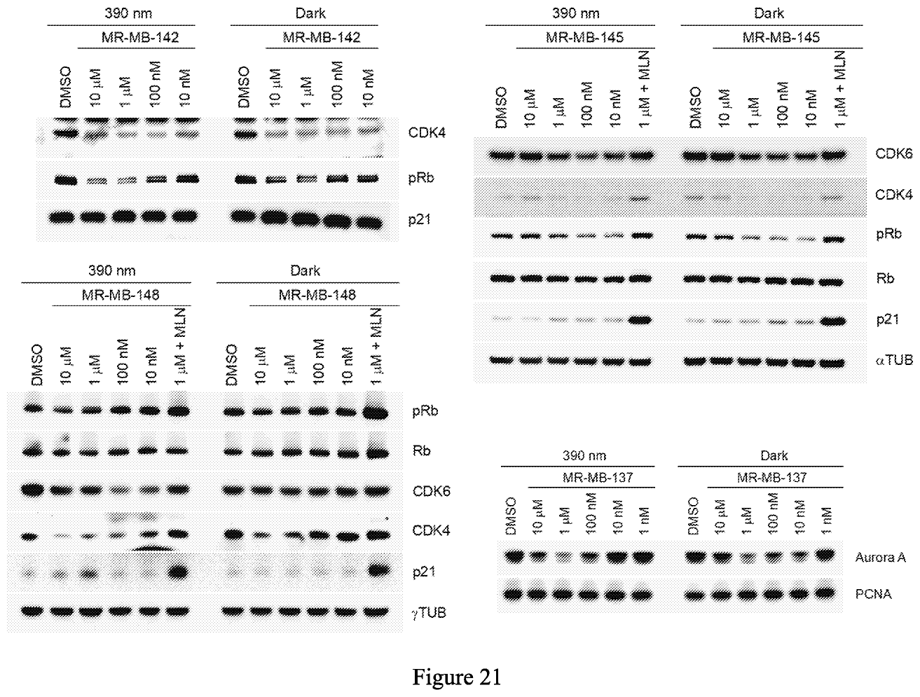

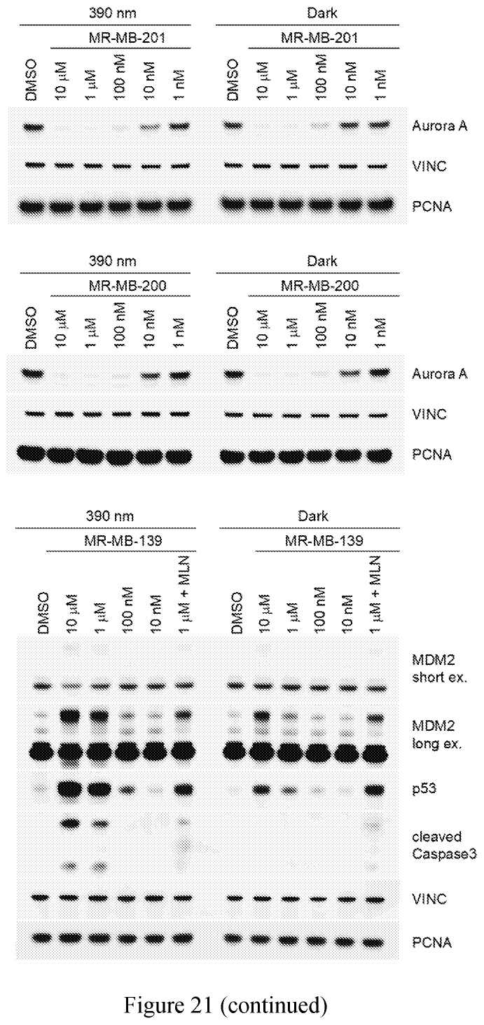

Amongst the molecules tested, PHOTAC-II-5 and PHOTAC-II-6 turned out to be the most useful, and the biological investigations have been focused on these molecules (for PHOTACs-II-1-4, see FIG. 16). PHOTAC-II-5, which has the azobenzene switch in the same position as PHOTACs-I-1-8, had a pronounced effect on FKBP12 levels upon pulse irradiation (FIG. 6A). The degradation was slower than in the case of BET proteins, but between 6 and 12 hours the protein was largely absent from the cell lysates (FIG. 6B). Again, no degradation could be observed in the dark. PHOTAC-II-6, wherein the photoswitch was moved further into the linker region, also elicited light-dependent degradation (FIG. 6C). The time course of FKBP12 degradation by PHOTAC-II-6 was similar to the one observed with PHOTAC-II-5 (FIG. 6D). In this case, however, slight dark activity at the 24-hour time point was also observed. Both PHOTACs showed a pronounced “hook effect” and were inactive in the presence of MLN4924.

By incorporating photoswitches into PROTACs, a general strategy was delineated to control targeted protein degradation with the temporal and spatial precision that light affords. As such, the concept of photopharmacology was applied to an important new target class, i.e., E3 ubiquitin ligases, and have added a highly useful functional feature to existing PROTACs.

As a proof of principle, PHOTACs that combine CRBN ligands with azobenzene photoswitches and ligands for either BET proteins (BRD2,3,4) or FKBP12 were developed. The modularity of the approach described herein should enable the straightforward development of PHOTACs that target many other classes of proteins. For instance, existing PROTACs that target CDK4/6, CDK9, BTK, ABL, ALK, MET, MDM2, and Tau could be adapted to become light activatable. PHOTACs for BRD2,3,4 may enable new insights into epigenetic pathways and potentially serve as precision tools in medicine. Color-dosing and reversibility could be particularity useful in this regard.

PHOTACs for FKBP12 could not only enable the optical degradation of wild type prolyl cis-trans isomerases, but also of proteins that are tagged with this domain. This would significantly increase the utility of dTAGs, which have emerged as a broadly applicable technology to influence the homeostasis of fusion proteins. In addition, PROTACs that link thalidomide derivatives to an alkyl halide could be modified to optically degrade HALO-tagged proteins. It should also be noted that some of the photoswitchable thalidomide derivatives, such as synthetic intermediates 2-4, and derivatives thereof, could function as dimerizers that recruit Ikaros (IKZF1) and Aiolos (IKZF3) to CRBN in a light-dependent fashion.

The PHOTACs introduced herein have several features that make them useful for biological studies: they are inactive as degraders in the dark and become active upon irradiation. Following activation, they gradually lose their activity through thermal relaxation. Alternatively, they can be quickly inactivated photochemically. In any scenario, their inactivation is much less dependent on dilution, clearance, or metabolism. In the case of compounds of the PHOTAC-I series, they still function as inhibitors of BET proteins, which explains the cytotoxicity observed in the dark. The concentrations needed for maximum photoeffect are low (nanomolar range), substantially reducing possible off-target effects. The light needed for photoactivation is not cytotoxic, given the low intensities needed for photoisomerization and the pulse protocol used.

In principle, light activation of PROTACs could also be achieved with a caging strategy. Indeed, caged PROTACs have very recently emerged, complementing PHOTACs. The advantage of the latter lies in their reversibility, the low intensities of light needed to trigger the photochemistry, the ease with which the active concentration can be tuned, and the avoidance of potentially toxic byproducts. Fast relaxing PHOTACs may also provide advantages in terms of localization and temporal control.

Genetically encoded degrons fused to a photosensitive LOV2 domain have been reported as an optogenetic approach to protein degradation. Although this approach works well in vivo and provides a powerful method to study biological pathways, it requires transfection with a gene of interest, which limits its therapeutic applicability and can potentially create unphysiological protein levels and distributions within a cell. PHOTACs, by contrast, operate like drugs in native tissues.

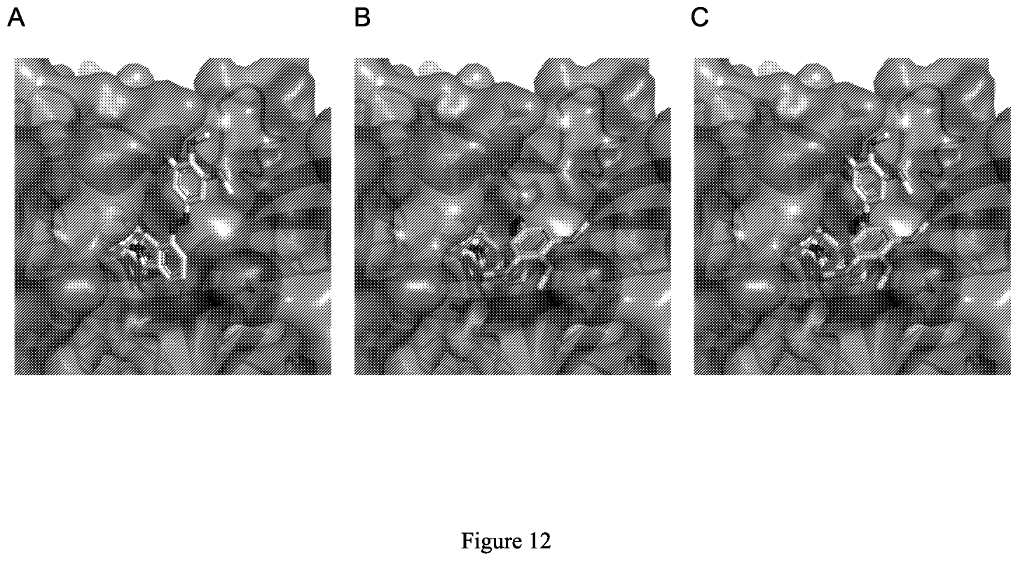

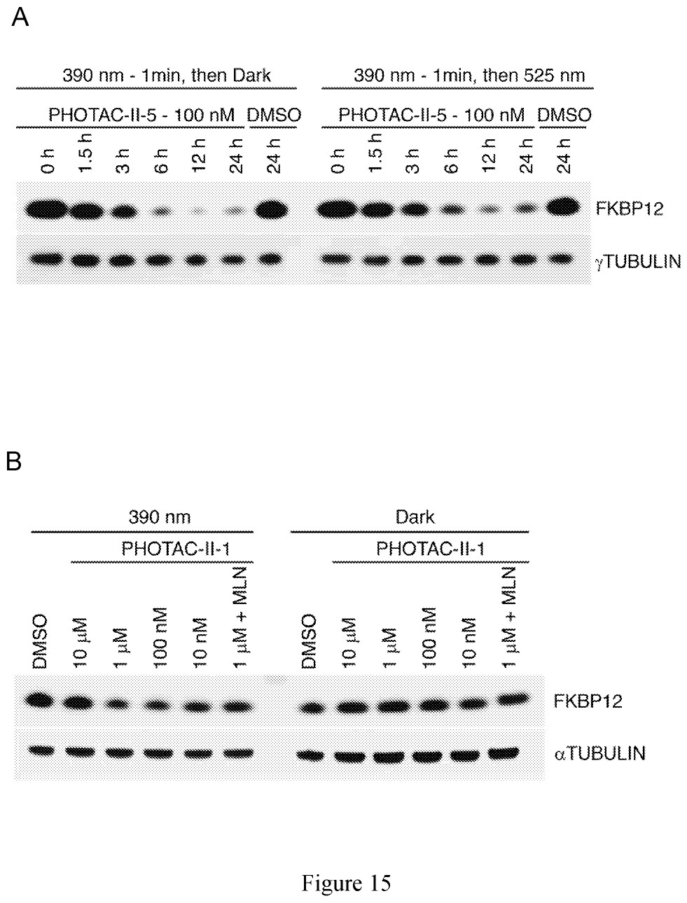

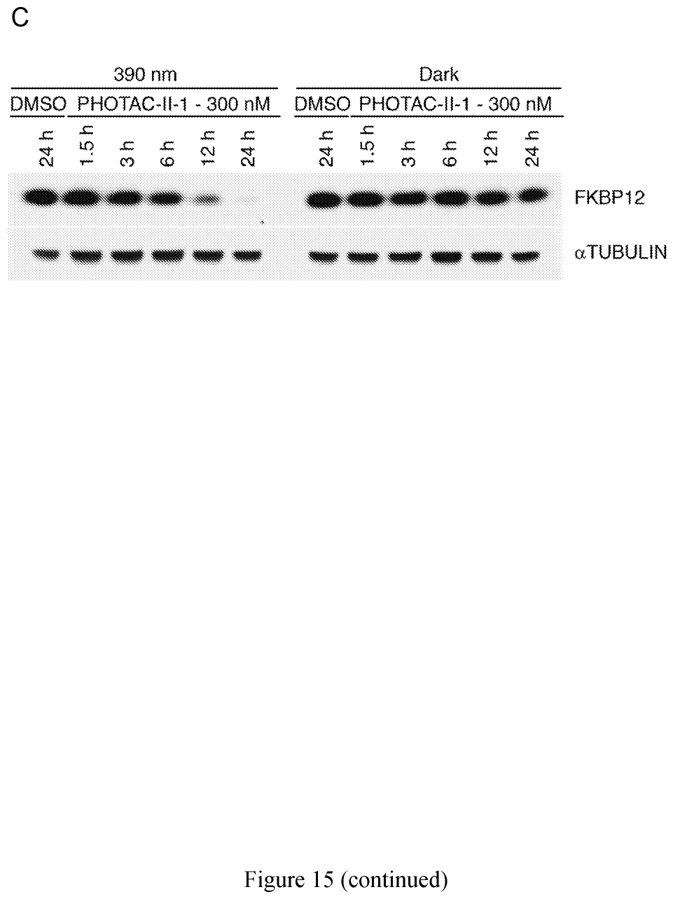

A question to address is how the (E)- and (Z)-isomers of PHOTACs influence ternary complex formation and target degradation. Although an effect of photoswitching on pharmacokinetics cannot be ruled out entirely, the increased activity as a degrader seems to be primarily driven by pharmacodynamics. A “hook effect” was consistently observed and no activity was found when CRBN was downregulated or in the presence of the neddylation inhibitor MLN4924, indicating that PHOTACs function as true PROTACs. Whether the photoswitch primarily affects binding to CRBN or the relative positioning of the E3 ligase and the POI remains to be determined. Molecular modeling suggests that the (E)-configuration of the photoswitch cannot be accommodated as well by cereblon as the (Z)-configuration (see FIG. 12). The importance of the exact nature, length, and orientation of the linker in PROTACs has been noted (“linkerology”). In keeping with this, a pronounced effect of the diamine spacers on the systems was observed. In the series targeting BET proteins, PHOTAC-I-3, which bears a 1,4-diamino butane spacer, showed the largest difference between light and dark activity in MTS assays. Related compounds that feature shorter or longer spacers, showed smaller or no differences (see FIG. 9). PHOTAC-I-10, wherein the photoswitch is positioned more centrally, also showed a light-dependent behavior, whereas analogs with different linker lengths did not. Amongst compounds targeting FKBP12, the lenalidomide derivative PHOTAC-II-5 and the thalidomide derivative PHOTAC-II-6, which feature different photoswitch incorporation modes, showed the largest light activation. In the case of derivative PHOTAC-II-5, longer linkers were detrimental to optical control (FIG. 15, 16).

PHOTACs can be taken into many different directions. Without intending to be bound by any particular theory, it is expected the incorporation of red-shifted and faster relaxing photoswitches could further improve the temporal and spatial precision of dark-inactive compounds. Synthetic approaches described herein are suited to increase photoswitch diversity. Different ligands for E3 ligases will be explored, for instance compounds that bind to VHL or the RING ligase MDM2. The optimal position of the photoswitches will depend on the exact nature of the system. Described herein are two different modes of photoswitch incorporation but a third variety (photoswitch in the POI ligand), and combinations thereof, will be explored. Without intending to be bound by any particular theory, the POI ligand should bind to its target without interfering with its function to cleanly distinguish between degradation and inhibition and to avoid unwanted toxicity.

Because PROTACs operate in a catalytic fashion and enable systemic protein knockdown, their toxicity is a major concern. PHOTACs of the type described herein may be activated with light within a tissue or before administration. They would then lose their activity with a given rate, which can be determined through engineering of the switch, or they could be actively turned off with a second wavelength. Moreover, PHOTACs locally activated by light would also lose efficacy by dilution diffusing away from the point of irradiation. The usefulness of light in medicine is well established and the combination of light and molecules has been studied for decades, e.g., in Photodynamic Therapy (PDT). PDT has been applied with encouraging results to treat non-small cell lung cancer, dermatological cancers, and premalignant lesions of the upper digestive tract, and is currently in clinical trials for the treatment of a large variety of other malignancies, including prostate, brain, and breast cancers. In the past, PDT was used both in cultured cells and in mouse models to induce death of prostate cancer cells in a calcium-dependent manner. However, from a molecular point of view, conventional PDT, while effective, is unspecific. PHOTACs, by contrast, can be reversibly activated with the temporal and spatial precision that light affords and target specific proteins whose elimination would promote cell death. Therefore, it is expected PHOTACs may be suitable in photomedicine.

Experimental—General methods. The reagents and solvents used in the present disclosure were bought from the following chemical suppliers (ABCR, Acros Organics, Alfa Aesar, Ark Pharm, Combi-Blocks, Oakwood, OxChem, Sigma-Aldrich, Strem, Toronto Research Chemicals) and were used as purchased.

Dry solvents used in reactions performed under inert atmosphere were obtained by passing the degassed solvents through activated alumina columns.

Column chromatography was carried out on silica gel ((60 A pore size, 40-63 μm, Merck KGaA)) using a Teledayne Isco Combiflash EZprep flash purification system.

Thin-layer chromatography (TLC) was performed on glass plates precoated with silica gel (0.25 mm, 60-A pore size, Merck). TLC plates were visualized by exposure to UV light (254 and 366 nm).

NMR spectra were obtained on a Bruker Avance III HD 400 MHz spectrometer equipped with a CryoProbe™ operating at 400 MHz for ¹H and 100 MHz for ¹³C spectra.

Multiplets are reported as observed and denoted as follows: s (singlet), d (doublet), t (triplet), q (quartet), p (pentet), h (hextet), and m (multiplet) and as combinations thereof.

High-resolution mass spectra (HRMS) were recorded on an Agilent Technologies 6224 Accurate-Mass time-of-flight spectrometer with either atmospheric pressure chemical ionization (APCI) or electrospray ionization (ESI) ionization sources.

LCMS were measured on an Agilent Technologies 1260 II Infinity connected to an Agilent Technologies 6120 Quadrupolemass spectrometer with ESI ionization source.

UV-Vis spectrometry was performed on a Varian Cary 60 UV-Visible Spectrophotometer using disposable BRAND UV-Cuvette Disposable Spectrophotometer/Photometer Ultra-Micro Cuvettes, BrandTech (10 mm light path), an Agilent Technologies PCB 1500 Water Peltier system for temperature control and samples were irradiated with a cairn research Optoscan Monochromator with Optosource High Intensity Arc Lamp equipped with a 75 W UXL-S50A lamp from USHIO Inc. Japan and set to 15 nm full width at half maximum.

### Experimental Procedures

### (E)-3-(4-((4-hydroxy-3,5-dimethoxyphenyl)diazenyl)-1-oxoisoindolin-2-yl)piperidine-2,6-dione (2)

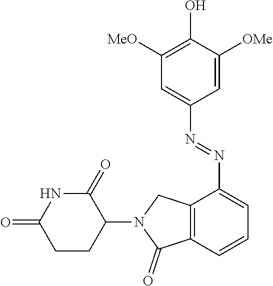 [SMILES: COc1cc(/N=N/c2cccc3c2CN(C2CCC(=O)NC2=O)C3=O)cc(OC)c1O]

Lenalidomide (500 mg, 1.93 mmol, 1.0 eq.) was dissolved in 1 M HCl (50 mL). Concentrated aqueous HBF₄ (2 mL, 48 wt. %) was added to the mixture. After completely dissolving of the starting material, 2 M NaNO₂ (1.06 mL) was added to the solution at 0° C. After stirring for 1 h the solution was added dropwise into a mixture of 2,6-Dimethoxyphenol (357 mg, 2.32 mmol, 1.2 eq.) in H₂O (50 mL), MeOH (20 mL), NaHCO₃ (4.000 g, 47.62 mmol, 24.7 eq.) and Na₂CO₃ (5.000 g, 47.18 mmol, 24.5 eq.). Upon addition the solution turned from violet to strong red and was stirred for 1 additional hour at 0° C. The reaction was extracted with EtOAc (7×100 mL) and washed once with brine (1×100 mL). The organic phase was dried over Na₂SO₄ and concentrated under reduced pressure. Purification of the resulting crude product by flash column chromatography (CH₂Cl₂/MeOH gradient, 0→10% MeOH) gave 2 (562.0 mg, 1.324 mmol, 69%) as a yellow solid. R_{f}=0.33 (CH₂Cl₂:MeOH, 19:1). ¹H NMR (400 MHz, DMSO-d₆) δ=11.03 (s, 1H), 9.48 (s, 1H), 8.16 (d, J=7.7 Hz, 1H), 7.87 (d, J=7.4 Hz, 1H), 7.77 (t, J=7.6 Hz, 1H), 7.34 (s, 2H), 5.16 (dd, J=13.1, 5.0 Hz, 1H), 4.82 (d, J=19.0 Hz, 1H), 4.69 (d, J=19.0 Hz, 1H), 3.90 (s, 6H), 2.95 (ddd, J=17.5, 13.4, 5.2 Hz, 1H), 2.63 (d, J=18.8 Hz, 1H), 2.55 (dd, J=13.1, 4.5 Hz, 1H), 2.11-2.03 (m, 1H) ppm. ¹³C NMR (100 MHz, DMSO-d₆) δ=173.39, 171.52, 167.76, 148.68, 147.05, 144.80, 140.86, 134.77, 134.21, 129.98, 128.37, 125.04, 101.69, 56.68, 52.30, 48.76, 31.76, 22.7 ppm. HRMS (ESI): calcd. for C₂₁H₂₁N₄O₆⁺: 425.1456 m/z [M+H]⁺; found: 425.1458 m/z [M+H]⁺. LCMS (ESI): tᵣₑₜ=2.90 min. 425 m/z [M+H]⁺.

### Tert-butyl (E)-2-(4-((2-(2,6-dioxopiperidin-3-yl)-1-oxoisoindolin-4-yl)diazenyl)-2,6-dimethoxyphenoxy)acetate (S1)

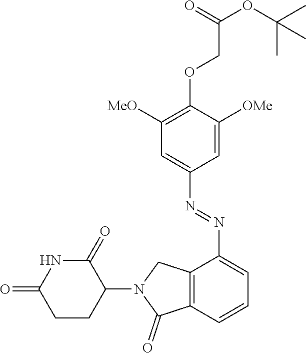 [SMILES: COc1cc(/N=N/c2cccc3c2CN(C2CCC(=O)NC2=O)C3=O)cc(OC)c1OCC(=O)OC(C)(C)C]

To tert-butyl bromoacetate (234 mg, 1.20 mmol, 1 eq.) was added dry DMF (10 mL), 2 (509 mg, 1.20 mmol, 1 eq.) and K2CO₃ (215 mg, 1.56 mmol, 1.3 eq.) at room temperature. After stirring for 2.5 hours, the mixture was diluted with EtOAc (100 mL), separated against NaHCO₃ (50 mL), extracted with EtOAc (3×50 mL) and washed with 10% LiCl (3×50 mL) and brine (2×50 mL). The reaction was concentrated under reduced pressure. Purification of the resulting crude product by flash column chromatography (Hx/Ea gradient, 20→100% Ea, the product was eluted at 75%.) gave 51 (501 mg, 0.93 mmol, 78%) as a yellow solid. R_{f}=0.64 [EtOAc]. ¹H NMR (400 MHz, DMSO-d₆) δ=11.03 (s, 1H), 8.22 (d, J=7.7 Hz, 1H), 7.92 (d, J=7.4 Hz, 1H), 7.80 (t, J=7.7 Hz, 1H), 7.34 (s, 2H), 5.16 (dd, J=13.2, 5.0 Hz, 1H), 4.83 (d, J=19.1 Hz, 1H), 4.70 (d, J=19.1 Hz, 1H), 4.60 (s, 2H), 3.90 (s, 6H), 2.93 (d, J=12.7 Hz, 1H), 2.63 (d, J=19.6 Hz, 1H), 2.58-2.52 (m, 1H), 2.11-2.03 (m, 1H), 1.43 (s, 9H) ppm. ¹³C NMR (100 MHz, DMSO-d₆) δ=173.38, 171.48, 168.16, 167.64, 152.82, 148.14, 146.86, 139.71, 134.96, 134.28, 130.08, 128.97, 125.78, 101.26, 81.47, 69.69, 56.77, 52.31, 48.78 31.75, 28.20, 22.77 ppm. HRMS (ESI): calcd. for C₂₇H₃₁N₄O₈⁺: 539.2137 m/z [M+H]⁺; found: 539.2172 m/z [M+H]⁺. LCMS (ESI): tᵣₑₜ=3.42 (Z). 539 m/z [M+H]⁺. tᵣₑₜ=3.93 (F) min. 539 m/z [M+H]⁺.

### (E)-2-(4-((2-(2,6-dioxopiperidin-3-yl)-1-oxoisoindolin-4-yl)diazenyl)-2,6-dimethoxyphenoxy)acetic Acid (3)

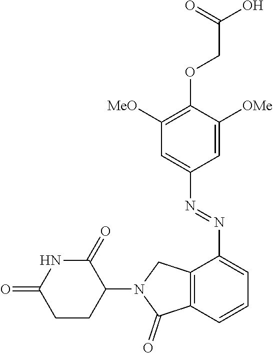 [SMILES: COc1cc(/N=N/c2cccc3c2CN(C2CCC(=O)NC2=O)C3=O)cc(OC)c1OCC(=O)O]

S2 (475 mg, 0.88 mmol, 1 eq.) was dissolved in CH₂Cl₂:TFA (1:1; 5 mL each). Upon TFA addition (5 mL) the solution turned from yellow to dark red. After 2 hours the reaction was concentrated under reduced pressure, turning from red to an orange solid. The reaction was dried under high vacuum for 48 h. 3 (557 mg, 0.878 mmol, 99%) was obtained as trifluoroacetate in form of a yellow solid with traces of residual TFA. R_{f}=0.5 [CH₂Cl₂:MeOH, 9:1]. ¹H NMR (400 MHz, DMSO-d₆) δ=11.03 (s, 1H), 8.22 (d, J=7.8 Hz, 1H), 7.92 (d, J=7.5 Hz, 1H), 7.80 (t, J=7.7 Hz, 1H), 7.34 (s, 2H), 5.16 (dd, J=13.2, 5.0 Hz, 1H), 4.83 (d, J=19.1 Hz, 1H), 4.70 (d, J=19.1 Hz, 2H), 4.60 (s, 2H), 3.90 (s, 6H), 2.95 (ddd, J=17.7, 13.6, 5.3 Hz, 1H), 2.68-2.53 (m, 2H), 2.11-2.03 (m, 1H). ppm. ¹³C NMR (100 MHz, DMSO) δ=172.91, 171.01, 169.95, 167.17, 152.58, 147.80, 146.41, 139.24, 134.50, 133.82, 129.63, 128.52, 125.35, 100.87, 68.67, 56.33, 51.85, 48.30, 31.28, 22.30 ppm. HRMS (ESI): calcd. for C₂₃H₂₃N₄O₈⁺: 483.1510 m/z [M+H]⁺; found: 483.1549 m/z [M+H]⁺. LCMS (ESI): tᵣₑₜ=2.93 min. 483.1 m/z [M+H]⁺.

### tert-butyl (E)-(2-(2-(4-((2-(2,6-dioxopiperidin-3-yl)-1-oxoisoindolin-4-yl)diazenyl)-2,6-dimethoxyphenoxy)acetamido)ethyl)carbamate (S4)

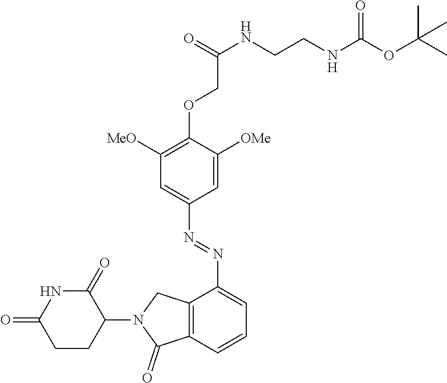 [SMILES: COc1cc(/N=N/c2cccc3c2CN(C2CCC(=O)NC2=O)C3=O)cc(OC)c1OCC(=O)NCCNC(=O)OC(C)(C)C]

3 (19.0 mg, 0.039 mmol, 1.0 eq.) and HATU (26.9 mg, 0.083 mmol, 2.1 eq.) were dissolved in dry DMF (1 mL) at room temperature. After 5 minutes of stirring N-Boc-1,2-ethylendiamine (33.2 mg, 0.207 mmol, 5.3 eq.) and i-Pr₂NEt (26.8 mg, 0.207 mmol, 5.3 eq., 36 μL) were added to the mixture and stirred for additional 12 h at room temperature. The reaction was diluted with EtOAc (20 mL), separated against H₂O (20 mL), extracted with EtOAc (3×20 mL) and washed with 10% LiCl (2×20 mL) and brine (3×30 mL). The combined organic phase was dried over Na₂SO₄ and concentrated under reduced pressure. Purification of the resulting crude product by flash column chromatography (CH₂Cl₂/MeOH gradient, 0-20% MeOH) gave S4 (15.3 mg, 0.024 mmol, 62%) as a yellow solid. R_{f}=0.32 [Hx:EA, 2:1]. ¹H NMR (400 MHz, Chloroform-0 6=8.21 (d, J=7.8 Hz, 1H), 8.02 (d, J=7.5 Hz, 1H), 7.99 (s, 1H), 7.89 (t, J=5.6 Hz, 1H, br), 7.72 (t, J=7.7 Hz, 1H), 7.22 (s, 2H), 5.27 (dd, J=13.3, 5.1 Hz, 1H), 4.86 (d, J=17.9 Hz, 1H), 4.75 (s, 1H), 4.62 (s, 2H), 4.01 (s, 6H), 3.49 (q, J=6.0 Hz, 2H), 3.32 (d, J=5.7 Hz, 2H), 3.00-2.81 (m, 2H), 2.47 (dd, J=13.1, 5.0 Hz, 1H), 2.27 (ddd, J=10.3, 5.1, 2.6 Hz, 1H), 1.44 (d, J=4.7 Hz, 9H) ppm. ¹³C NMR (100 MHz, CDCl₃) δ=170.98, 170.37, 169.51, 168.57, 156.18, 152.79, 149.09, 146.93, 139.86, 134.12, 133.45, 130.05, 129.66, 126.44, 100.65, 79.66, 72.84, 56.60, 52.15, 48.29, 39.17, 31.76, 28.54, 23.62, 19.18 ppm. HRMS (ESI): calcd. for C₃₀H₃₇N₆O₉⁺: 265.2617 m/z [M+H]⁺; found: 265.2629 m/z [M+H]⁺. LCMS (ESI): tᵣₑₜ=3.11 (Z) min. 623 m/z [M−H]⁻. tᵣₑₜ=3.44 (E) min. 623 m/z [M−H]⁻.

### tert-butyl (E)-(3-(2-(4-((2-(2,6-dioxopiperidin-3-yl)-1-oxoisoindolin-4-yl)diazenyl)-2,6-dimethoxyphenoxy)acetamido)propyl)carbamate (S5)

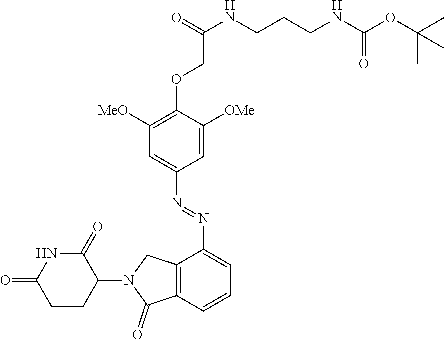 [SMILES: COc1cc(/N=N/c2cccc3c2CN(C2CCC(=O)NC2=O)C3=O)cc(OC)c1OCC(=O)NCCCNC(=O)OC(C)(C)C]

3 (60.0 mg, 0.095 mmol, 1.0 eq.) and HATU (46.0 mg, 0.142 mmol, 1.5 eq.) were dissolved in dry DMF (5 mL) at room temperature. After 5 minutes of stirring N-Boc-1,3-diaminopropane (65.9 mg, 0.378 mmol, 4.0 eq. 70 μl) and i-Pr₂NEt (48.9 mg, 0.378 mmol, 4.0 eq., 66 μl) were added to the mixture and stirred for additional 12 h at room temperature. The reaction was diluted with EtOAc (20 mL), separated against H₂O/10% LiCl (1:1, 10 mL:10 mL), extracted with EtOAc (2×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave S5 (51.5 mg, 0.081 mmol, 86%) as a yellow solid. R_{f}=0.19 [CH₂Cl₂:MeOH, 9:1]. ¹H NMR (400 MHz, Chloroform-d) δ=8.21 (d, J=7.8 Hz, 1H), 8.02 (d, J=7.5 Hz, 1H), 7.99 (s, 1H), 7.73 (d, J=7.7 Hz, 1H), 7.70 (s, 1H), 7.22 (s, 2H), 5.27 (dd, J=13.3, 5.1 Hz, 1H), 5.01 (s, 1H), 4.86 (d, J=17.9 Hz, 1H), 4.72 (d, J=17.9 Hz, 1H), 4.60 (s, 2H), 4.00 (s, 6H), 3.43 (q, J=6.5 Hz, 2H), 3.18 (d, J=6.3 Hz, 2H), 2.99-2.81 (m, 2H), 2.47 (dd, J=13.1, 5.0 Hz, 1H), 2.27 (dtd, J=12.8, 5.1, 2.5 Hz, 1H), 1.74 (p, J=6.6 Hz, 1H), 1.43 (s, 9H) ppm. ¹³C NMR (100 MHz, CDCl₃) δ=170.99, 170.05, 169.51, 168.57, 156.22, 152.82, 149.07, 146.93, 139.88, 134.11, 133.45, 130.06, 129.66, 126.42, 100.61, 79.22, 72.83, 56.54, 52.15, 48.29, 37.68, 36.24, 31.76, 30.30, 28.57, 23.63 ppm. HRMS (ESI): calcd. for C₃₁H₃₉N₆O₉⁺: 639.2773 m/z [M+H]⁺; found: 639.2785 m/z [M+H]⁺. LCMS (ESI): tᵣₑₜ=3.23 min (Z). 639.2 m/z [M+H]⁺.

### tert-butyl (E)-(4-(2-(4-((2-(2,6-dioxopiperidin-3-yl)-1-oxoisoindolin-4-yl)diazenyl)-2,6-dimethoxyphenoxy)acetamido)butyl)carbamate (S3)

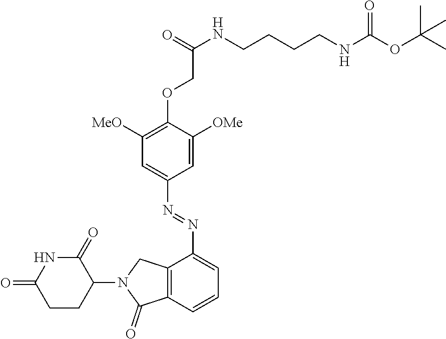 [SMILES: COc1cc(/N=N/c2cccc3c2CN(C2CCC(=O)NC2=O)C3=O)cc(OC)c1OCC(=O)NCCCCNC(=O)OC(C)(C)C]

3 (70.0 mg, 0.11 mmol, 1.0 eq.) and HATU (62.9 mg, 0.165 mmol, 1.5 eq.) were dissolved in dry DMF (1 mL) at room temperature. After 5 minutes of stirring N-Boc-1,4-diaminobutane (83.1 mg, 0.441 mmol, 4 eq.) and i-Pr₂NEt (57 mg, 0.44 mmol, 4 eq., 74 μL) were added to the mixture and stirred for additional 14 h at room temperature. The reaction was diluted with EtOAc (20 mL), separated against 5% LiCl (20 mL), extracted with EtOAc (3×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave S3 (53.7 mg, 0.082 mmol, 75%) as a yellow solid. R_{f}=0.42 [CH₂Cl₂:MeOH, 19:1]. ¹H NMR (400 MHz, Chloroform-d) δ=8.21 (d, J=7.7 Hz, 1H), 8.10 (s, 1H), 8.01 (d, J=7.4 Hz, 1H), 7.72 (t, J=7.7 Hz, 1H), 7.69 (s, 1H), 7.22 (s, 2H), 5.26 (dd, J=13.3, 5.1 Hz, 1H), 4.86 (d, J=17.9 Hz, 1H), 4.72 (d, J=17.9 Hz, 1H), 4.60 (s, 2H), 3.99 (s, 6H), 3.37 (q, J=6.5 Hz, 2H), 3.17 (d, J=5.9 Hz, 2H), 2.99-2.81 (m, 2H), 2.47 (dd, J=13.1, 5.1 Hz, 1H), 2.27 (dtd, J=12.8, 7.6, 6.3, 3.7 Hz, 1H), 1.66-1.53 (m, 4H), 1.43 (s, 9H) ppm. ¹³C NMR (100 MHz, CDCl₃) δ=171.04, 169.58, 169.55, 168.57, 156.14, 152.76, 149.02, 146.92, 139.91, 134.12, 133.45, 130.03, 129.64, 126.41, 100.60, 79.36, 72.89, 56.52, 52.15, 48.30, 40.39, 38.80, 31.76, 28.56, 27.73, 27.13, 23.62 ppm. HRMS (APCI): calcd. for C₃₁H₄₁N₆O₉⁺: 653.2929 m/z [M+H]⁺; found: 653.2928.9606 m/z [M+H]⁺. LCMS (ESI): tᵣₑₜ=3.31 min (Z) 675 m/z [M+Na]⁺. tᵣₑₜ=3.63 min (E) 675 m/z [M+Na]⁺.

### Tert-butyl (E)-(5-(2-(4-((2-(2,6-dioxopiperidin-3-yl)-1-oxoisoindolin-4-yl)diazenyl)-2,6-dimethoxyphenoxy)acetamido)pentyl)carbamate (S6)

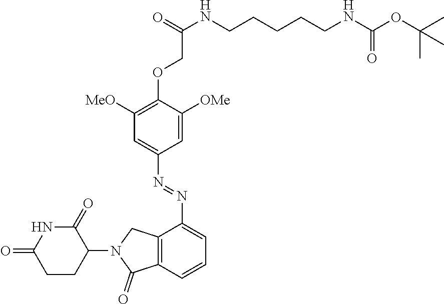 [SMILES: COc1cc(/N=N/c2cccc3c2CN(C2CCC(=O)NC2=O)C3=O)cc(OC)c1OCC(=O)NCCCCCNC(=O)OC(C)(C)C]

3 (60.0 mg, 0.095 mmol, 1.0 eq.) and HATU (46.0 mg, 0.142 mmol, 1.5 eq.) were dissolved in dry DMF (5 mL) at room temperature. After 5 minutes of stirring N-Boc-1,4-diaminopentane (76.9 mg, 0.380 mmol, 4 eq.) and i-Pr₂NEt (49.1 mg, 0.380 mmol, 4 eq., 66 μl) were added to the mixture and stirred for additional 12 h at room temperature. The reaction was diluted with EtOAc (20 mL), separated against 5% LiCl (20 mL), extracted with EtOAc (3×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) yielded S6 (54.2 mg, 0.081 mmol, 85%) as a yellow solid. R_{f}=0.42 [CH₂Cl₂:MeOH, 19:1]. ¹H NMR (400 MHz, Chloroform-d) δ=8.21 (d, J=7.8 Hz, 1H), 8.14 (d, J=13.9 Hz, 1H), 8.01 (d, J=7.5 Hz, 1H), 7.72 (t, J=7.7 Hz, 1H), 7.66 (s, 1H), 7.22 (s, 2H), 5.26 (dd, J=13.3, 5.1 Hz, 1H), 4.86 (d, J=17.9 Hz, 1H), 4.73 (d, J=17.9 Hz, 1H), 4.61 (s, 2H), 3.99 (s, 6H), 3.35 (q, J=6.8 Hz, 2H), 3.12 (d, J=5.7 Hz, 3H), 3.00-2.81 (m, 2H), 2.47 (qd, J=13.1, 5.0 Hz, 1H), 2.30-2.21 (m, 1H), 1.67-1.48 (m, 6H), 1.43 (s, 9H) ppm. ¹³C NMR (100 MHz, CDCl₃) δ=171.11, 171.04, 169.54, 168.57, 156.14, 152.77, 149.02, 146.93, 139.93, 134.10, 133.44, 130.07, 129.64, 126.39, 100.60, 79.29, 72.90, 56.51, 52.15, 48.31, 40.52, 38.96, 31.76, 29.92, 29.45, 28.56, 24.23, 23.62 ppm. HRMS (ESI): calcd. for C₃₃H₄₂N₆NaO₉⁺: 689.2905 m/z [M+Na]⁺; found: 689.2935 m/z [M+Na]⁺. LCMS (ESI): tᵣₑₜ=3.43 min (Z). 665 m/z [M−H]⁻.

### tert-butyl (E)-(6-(2-(4-((2-(2,6-dioxopiperidin-3-yl)-1-oxoisoindolin-4-yl)diazenyl)-2,6-dimethoxyphenoxy)acetamido)hexyl)carbamate (S7)

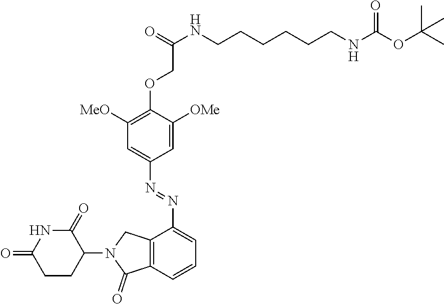 [SMILES: COc1cc(/N=N/c2cccc3c2CN(C2CCC(=O)NC2=O)C3=O)cc(OC)c1OCC(=O)NCCCCCCNC(=O)OC(C)(C)C]

3 (60.0 mg, 0.095 mmol, 1.0 eq.) and HATU (46.0 mg, 0.142 mmol, 1.5 eq.) were dissolved in dry DMF (5 mL) at room temperature. After 5 minutes of stirring N-Boc-1,4-diaminohexane (81.8 mg, 0.378 mmol, 4 eq., 0.09 mL) and i-Pr₂NEt (48.9 mg, 0.378 mmol, 4 eq., 66 μL) were added to the mixture and stirred for additional 13 h at room temperature. The reaction was diluted with EtOAc (20 mL), separated against 5% LiCl (20 mL), extracted with EtOAc (3×20 mL) and washed twice with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave S7 (56.7 mg, 0.083 mmol, 88%) as a yellow solid. R_{f}=0.25 [CH₂Cl₂: MeOH, 19:1]. ¹H NMR (400 MHz, Chloroform-d) δ=8.20 (d, J=7.8 Hz, 2H), 8.01 (d, J=7.5 Hz, 1H), 7.71 (t, J=7.7 Hz, 1H), 7.64 (t, J=5.3 Hz, 1H), 7.21 (s, 2H), 5.26 (dd, J=13.3, 5.1 Hz, 1H), 4.86 (d, J=17.9 Hz, 1H), 4.72 (d, J=17.9 Hz, 1H), 4.61 (s, 2H), 3.99 (s, 6H), 3.34 (q, J=6.6 Hz, 2H), 3.10 (d, J=6.1 Hz, 2H), 2.99-2.80 (m, 2H), 2.46 (qd, J=13.1, 5.0 Hz, 1H), 2.26 (dtd, J=12.8, 5.1, 2.6 Hz, 1H), 1.58 (p, J=7.1 Hz, 2H), 1.52-1.46 (m, 2H), 1.43 (s, 9H), 1.40-1.34 (m, 4H) ppm. ¹³C NMR (100 MHz, CDCl₃) δ=171.16, 169.64, 169.48, 168.56, 156.14, 152.76, 148.99, 146.92, 139.93, 134.08, 133.44, 130.08, 129.63, 126.38, 100.59, 79.25, 72.89, 56.50, 52.13, 48.29, 40.64, 39.02, 31.76, 30.17, 29.67, 28.57, 26.74, 26.63, 23.63 ppm. HRMS (APCI): calcd. for C₃₄H₄₅N₆O₉⁺: 681.3243 m/z [M+H]⁺; found: 681.3236 m/z [M+H]⁺.

### (E)-(2-(2-(4-((2-(2,6-dioxopiperidin-3-yl)-1-oxoisoindolin-4-yl)diazenyl)-2,6-dimethoxyphenoxy)acetamido)ethyl)carbamic Acid (S8)

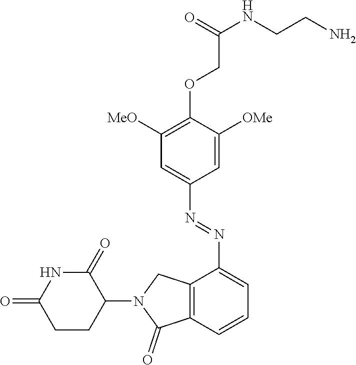 [SMILES: COc1cc(/N=N/c2cccc3c2CN(C2CCC(=O)NC2=O)C3=O)cc(OC)c1OCC(=O)NCCN]

S4 (15.3 mg, 0.024 mmol, 1 eq.) was dissolved in TFA:CH₂Cl₂ (1 mL:0.5 mL) and stirred at room temperature. After 2 h, the mixture was diluted with CH₂Cl₂, concentrated under reduced pressure and dried on high vacuum overnight. S8 (15.6 mg, 0.024 mmol, >99%.) was obtained as trifluoroacetate in form of a yellow solid with traces of residual TFA. R_{f}=0.18 [CH₂Cl₂:MeOH, 7:1]. ¹H NMR (400 MHz, DMSO-d₆) δ=11.04 (s, 1H), 8.23 (dd, J=9.3, 7.1 Hz, 2H), 7.93 (d, J=7.5 Hz, 1H), 7.81 (t, J=7.7 Hz, 3H), 7.37 (s, 2H), 5.17 (dd, J=13.3, 5.0 Hz, 1H), 4.82 (d, J=19.1 Hz, 1H), 4.69 (d, J=19.1 Hz, 1H), 4.44 (s, 2H), 3.94 (s, 6H), 3.45 (q, J=6.3 Hz, 2H), 3.02-2.88 (m, 4H), 2.69-2.54 (m, 2H) ppm. ¹³C NMR (100 MHz, DMSO-d₆) δ=172.92, 171.02, 169.13, 167.14, 152.77, 148.38, 146.35, 139.08, 134.49, 133.84, 129.68, 128.67, 125.54, 100.57, 71.67, 56.36, 51.83, 48.26, 38.80, 36.23, 31.28, 30.70 ppm. HRMS (ESI): calcd. for C₂₅H₂₉N₆O₇⁺: 525.2092 m/z [M+H]⁺; found: 525.2096 m/z [M+H]⁺. LCMS (ESI): tᵣₑₜ=2.39 min. 525 m/z [M+H]⁺.

### (E)-N-(3-aminopropyl)-2-(4-((2-(2,6-dioxopiperidin-3-yl)-1-oxoisoindolin-4-yl)diazenyl)-2,6-dimethoxyphenoxy)acetamide (S9)

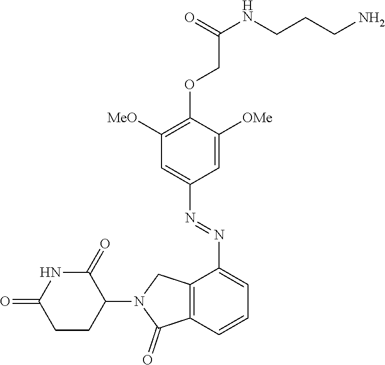 [SMILES: COc1cc(/N=N/c2cccc3c2CN(C2CCC(=O)NC2=O)C3=O)cc(OC)c1OCC(=O)NCCCN]

S5 (41.5 mg, 0.061 mmol, 1 eq.) was dissolved in TFA/CH₂Cl₂ (1:1, 1 mL:1 mL) and stirred for 2 h at room temperature. The reaction was diluted with MeOH and concentrated under reduced pressure. The mixture was triturated with CH₂Cl₂ and dried on high vacuum overnight. S9 (40 mg, 0.061 mmol, >99%) was obtained as a yellow solid with traces of residual TFA. R_{f}=0.19 [CH₂Cl₂:MeOH, 9:1]. ¹H NMR (400 MHz, DMSO-d₆) δ=11.04 (s, 1H), 8.22 (d, J=7.8 Hz, 1H), 8.16 (t, J=5.9 Hz, 1H), 7.93 (d, J=7.5 Hz, 1H), 7.81 (t, J=7.7 Hz, 1H), 7.73 (s, 2H), 7.36 (s, 2H), 5.17 (dd, J=13.2, 5.0 Hz, 1H), 4.82 (d, J=19.1 Hz, 1H), 4.69 (d, J=19.1 Hz, 1H), 4.43 (s, 2H), 3.94 (s, 6H), 3.27 (q, J=6.5 Hz, 2H), 2.95 (ddd, J=17.9, 13.8, 5.2 Hz, 1H), 2.82 (dt, J=12.9, 6.2 Hz, 2H), 2.69-2.53 (m, 2H), 2.11-2.03 (m, 1H), 1.77 (p, J=6.9 Hz, 2H) ppm. ¹³C NMR (100 MHz, DMSO) δ=172.92, 171.02, 168.55, 167.15, 152.72, 148.34, 146.35, 139.15, 134.48, 133.84, 129.68, 128.70, 125.54, 100.60, 71.72, 56.39, 51.83, 48.27, 36.75, 35.36, 31.28, 27.39, 22.34 ppm. HRMS (ESI): calcd. for C₂₆H₃₁N₆O₇⁺: 539.2249 m/z [M+H]⁺; found: 539.2312 m/z [M+H]⁺. LCMS (ESI): tᵣₑₜ=2.34 min (Z). 539 m/z [M+H]⁺. tᵣₑₜ=2.45 min (E). 539 m/z [M+H]⁺.

### (E)-N-(4-aminobutyl)-2-(4-((2-(2,6-dioxopiperidin-3-yl)-1-oxoisoindolin-4-yl)diazenyl)-2,6-dimethoxyphenoxy)acetamide (4)

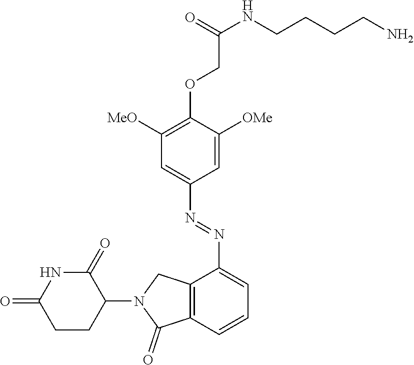 [SMILES: COc1cc(/N=N/c2cccc3c2CN(C2CCC(=O)NC2=O)C3=O)cc(OC)c1OCC(=O)NCCCCN]

S3 (39.5 mg, 0.057 mmol, 1 eq.) was dissolved in TFA/CH₂Cl₂ (1:1; 1 mL:1 mL) and stirred for 2 h at room temperature. The reaction was diluted with CH₂Cl₂ and concentrated under reduced pressure. The reaction was triturated with Et₂O and dried on high vacuum overnight. 4 (38 mg, 0.057 mmol, >99%) was obtained as trifluoroacetate in form of a yellow solid with traces of residual TFA. R_{f}=0.4 [CH₂Cl₂:MeOH, 8:2]. ¹H NMR (400 MHz, DMSO-d₆) δ=11.04 (s, 1H), 8.22 (d, J=7.8 Hz, 1H), 8.02 (t, J=5.8 Hz, 1H), 7.93 (d, J=7.5 Hz, 1H), 7.81 (t, J=7.7 Hz, 1H), 7.70 (s, 2H), 7.37 (s, 2H), 5.17 (dd, J=13.2, 5.0 Hz, 1H), 4.82 (d, J=19.1 Hz, 1H), 4.69 (d, J=19.1 Hz, 1H), 4.42 (s, 2H), 3.95 (s, 6H), 3.25-3.19 (m, 2H), 2.95 (ddd, J=17.9, 13.7, 5.3 Hz, 1H), 2.86-2.79 (m, 2H), 2.67-2.53 (m, 2H), 2.11-2.03 (m, 1H), 1.58-1.51 (m, 4H) ppm. ¹³C NMR (100 MHz, DMSO) δ=172.92, 171.02, 168.11, 167.15, 152.66, 148.34, 146.35, 139.20, 134.48, 133.84, 129.68, 128.70, 125.54, 100.59, 71.83, 56.39, 51.83, 48.28, 38.58, 37.58, 31.28, 26.13, 24.46, 22.33 ppm. HRMS (APCI): calcd. for C₂₇H₃₃N₆O₉⁺: 553.2405 m/z [M+H]⁺; found: 553.2384 m/z [M+H]⁺. LCMS (ESI): tᵣₑₜ=2.23 min (Z). 553 m/z [M+H]⁺. tᵣₑₜ=2.49 min (E). 553 m/z [M+H]⁺.

### (E)-N-(5-aminopentyl)-2-(4-((2-(2,6-dioxopiperidin-3-yl)-1-oxoisoindolin-4-yl)diazenyl)-2,6-dimethoxyphenoxy)acetamide (S10)

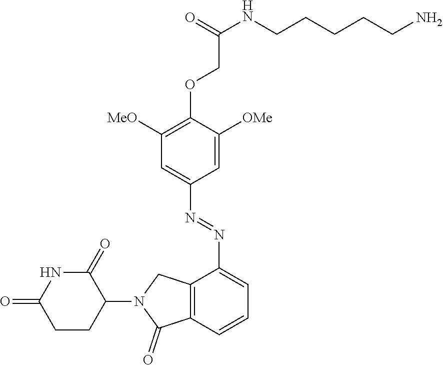 [SMILES: COc1cc(/N=N/c2cccc3c2CN(C2CCC(=O)NC2=O)C3=O)cc(OC)c1OCC(=O)NCCCCCN]

S6 (40.5 mg, 0.057 mmol, 1 eq.) was dissolved in TFA/CH₂Cl₂ (1:1; 1 mL:1 mL) and stirred for 2 h at room temperature. The reaction was diluted with CH₂Cl₂ and concentrated under reduced pressure. The reaction was triturated with Et₂O and dried on high vacuum overnight. S10 (38.8 mg, 0.057 mmol, >99%) was obtained as trifluoroacetate in form of a yellow solid with traces of residual TFA. R_{f}=0.03 [CH₂Cl₂:MeOH, 8:2]. ¹H NMR (400 MHz, DMSO-d₆) δ=11.03 (s, 1H), 8.22 (d, J=7.8 Hz, 1H), 7.97 (d, J=5.7 Hz, 1H), 7.93 (d, J=7.5 Hz, 1H), 7.81 (t, J=7.7 Hz, 1H), 7.68 (s, 2H), 7.36 (s, 2H), 5.17 (dd, J=13.2, 5.0 Hz, 1H), 4.82 (d, J=19.1 Hz, 1H), 4.69 (d, J=19.1 Hz, 1H), 4.42 (s, 2H), 3.95 (s, 6H), 3.19 (q, J=6.7 Hz, 2H), 2.95 (ddd, J=17.9, 13.7, 5.3 Hz, 1H), 2.79 (h, J=5.8 Hz, 2H), 2.68-2.53 (m, 2H), 2.11-2.03 (m, 1H), 1.53 (dp, J=22.1, 7.5 Hz, 4H), 1.33 (p, J=7.5, 6.9 Hz, 2H) ppm. ¹³C NMR (100 MHz, DMSO) δ=172.92, 171.02, 168.00, 167.15, 152.64, 148.31, 146.35, 139.24, 134.49, 133.84, 129.67, 128.68, 125.53, 100.61, 71.85, 56.38, 51.83, 48.28, 38.76, 37.92, 31.29, 28.59, 26.67, 23.12, 22.33 ppm. HRMS (ESI): calcd. for C₂₈H₃₅N₆O₇⁺: 567.2562 m/z [M+H]⁺; found: 567.2656 m/z [M+H]⁺. LCMS (ESI): tᵣₑₜ=2.27 min (Z). 567 m/z [M+H]⁺. tᵣₑₜ=2.53 min (E). 67 m/z [M+H]⁺.

### (E)-N-(6-aminohexyl)-2-(4-((2-(2,6-dioxopiperidin-3-yl)-1-oxoisoindolin-4-yl)diazenyl)-2,6-dimethoxyphenoxy)acetamide (S11)

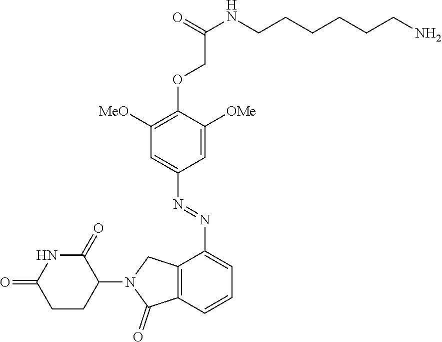 [SMILES: COc1cc(/N=N/c2cccc3c2CN(C2CCC(=O)NC2=O)C3=O)cc(OC)c1OCC(=O)NCCCCCCN]

S7 (51.3 mg, 0.071 mmol, 1 eq.) was dissolved in TFA/CH₂Cl₂ (1:1; 1 mL:1 mL) and stirred for 2 h at room temperature. The reaction was diluted with CH₂Cl₂ and concentrated under reduced pressure. The reaction was triturated with Et₂O and dried on high vacuum overnight. S11 (49.3 mg, 0.071 mmol, >99%) was obtained as trifluoroacetate in form of a yellow solid with traces of residual TFA. R_{f}=0.19 [CH₂Cl₂: MeOH, 9:1]. ¹H NMR (400 MHz, DMSO-d₆) δ=11.03 (s, 1H), 8.22 (d, J=7.8 Hz, 1H), 7.94 (dd, J=11.5, 6.5 Hz, 2H), 7.81 (t, J=7.7 Hz, 1H), 7.66 (s, 2H), 7.36 (s, 2H), 5.16 (dd, J=13.2, 5.0 Hz, 1H), 4.82 (d, J=19.1 Hz, 1H), 4.69 (d, J=19.1 Hz, 1H), 4.42 (s, 2H), 3.94 (s, 6H), 3.19 (q, J=6.6 Hz, 2H), 2.95 (ddd, J=17.9, 13.8, 5.3 Hz, 1H), 2.78 (h, J=5.8 Hz, 2H), 2.63 (d, J=18.1 Hz, 1H), 2.56 (dd, J=13.1, 4.4 Hz, 1H), 2.06 (d, J=5.4 Hz, 1H), 1.51 (dp, J=13.7, 6.9 Hz, 4H), 1.37-1.25 (m, 4H) ppm. ¹³C NMR (100 MHz, DMSO) δ=172.95, 171.04, 167.99, 167.18, 152.64, 148.32, 146.37, 139.28, 134.51, 133.85, 129.70, 128.71, 125.56, 100.63, 71.89, 56.40, 51.86, 48.31, 38.82, 38.09, 31.30, 28.95, 26.98, 25.84, 25.48, 22.35 ppm. HRMS (APCI): calcd. for C₂₉H₃₇N₆O₇⁺: 581.2718 m/z [M+H]⁺; found: 581.2717 m/z [M+H]⁺. LCMS (ESI): tᵣₑₜ=2.66 min. 581 m/z [M+H]⁺.

### (S)-2-(4-(4-chlorophenyl)-2,3,9-trimethyl-6H-thieno[3,2-f][1,2,4]triazolo[4,3-a][1,4]diazepin-6-yl)acetic Acid ((+)-JQ1 Free Acid, 5)

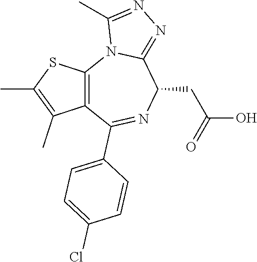 [SMILES: Cc1sc2c(c1C)C(c1ccc(Cl)cc1)=N[C@@H](CC(=O)O)c1nnc(C)n1-2]

As previously described, (+)-JQ1 (9 mg, 0.02 mmol, 1 eq.) was dissolved in formic acid (0.5 mL) and stirred for 3 days at room temperature. The reaction was concentrated under reduced pressure and was dried on high vacuum overnight. (+)-JQ1 free acid, 5 (8.0 mg, 0.02 mmol, >99%) was obtained as yellow solid and used without further purification. LCMS (ESI): tᵣₑₜ=3.35 min. 401, 402 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(2-(2-(4-((E)-(2-(2,6-dioxopiperidin-3-yl)-1-oxoisoindolin-4-yl)diazenyl)-2,6-dimethoxyphenoxy)acetamido)ethyl)acetamide (PHOTAC-I-1)

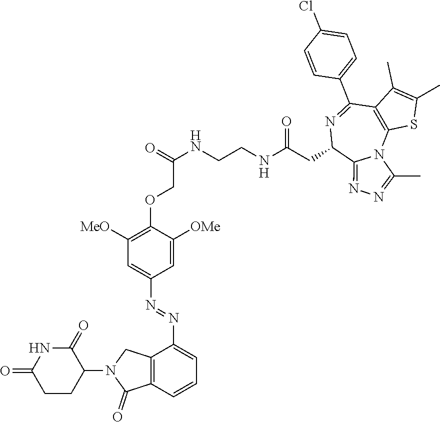 [SMILES: COc1cc(/N=N/c2cccc3c2CN(C2CCC(=O)NC2=O)C3=O)cc(OC)c1OCC(=O)NCCNC(=O)C[C@@H]1N=C(c2ccc(Cl)cc2)c2c(sc(C)c2C)-n2c(C)nnc21]

S8 (15.3 mg, 0.024 mmol, 1 eq.) and HATU (11.7 mg, 0.036 mmol, 1.5 eq.) were added to a round bottom flask under nitrogen. (+)-JQ1 free acid was taken up in dry DMF (1 mL) and added to the mixture. After addition of i-Pr₂NEt (0.025 mL, 0.144 mmol, 6 eq.) the reaction was stirred for 15 h at room temperature. Then the mixture was diluted with EtOAc (20 mL), separated against H₂O (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-1 (8.6 mg, 0.009 mmol, 38%) as a yellow solid. R_{f}=0.19 [CH₂Cl₂:MeOH, 19:1]. ¹H NMR (400 MHz, Methanol-d₄) δ=8.20 (d, J=7.8 Hz, 1H), 7.90 (dd, J=7.5, 4.1 Hz, 1H), 7.74 (td, J=7.7, 2.8 Hz, 1H), 7.40 (dd, J=8.7, 3.0 Hz, 2H), 7.36-7.28 (m, 4H), 5.23-5.09 (m, 1H), 4.83 (s, 2H), 4.60-4.47 (m, 3H), 3.96 (d, J=2.8 Hz, 6H), 3.58-3.33 (m, 5H), 3.30-3.19 (m, 1H), 2.92 (ddt, J=17.9, 13.4, 4.8 Hz, 1H), 2.79 (ddt, J=17.8, 5.1, 2.8 Hz, 1H), 2.70-2.61 (m, 1H), 2.58 (d, J=19.6 Hz, 3H), 2.40 (d, J=2.2 Hz, 3H), 2.25-2.14 (m, 1H), 1.66 (s, 3H). ¹³C NMR (100 MHz, MeOD) δ=174.68, 173.16, 172.58, 172.18, 170.35, 166.19, 156.89, 154.11, 152.07, 150.18, 148.12, 141.02, 138.01, 137.92, 135.56, 134.82, 133.42, 133.14, 132.07, 131.92, 131.57, 131.30, 130.69, 129.74, 126.55, 101.71, 73.23, 56.97, 54.99, 53.91, 50.68, 40.04, 39.91, 38.72, 32.42, 24.03, 14.39, 12.93, 11.58 ppm. HRMS (ESI): calcd. for C₄₄H₄₄CIN₁₀O₈S⁺: 907.2747 m/z [M+H]⁺; found: 907.2790 m/z [M+H]⁺. LCMS (ESI): tᵣₑₜ=3.51 min (Z). 907 m/z [M+H]⁺. tᵣₑₜ=3.67 min (E). 907 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(3-(2-(4-((E)-(2-(2,6-dioxopiperidin-3-yl)-1-oxoisoindolin-4-yl)diazenyl)-2,6-dimethoxyphenoxy)acetamido)propyl)acetamide (PHOTAC-I-2)

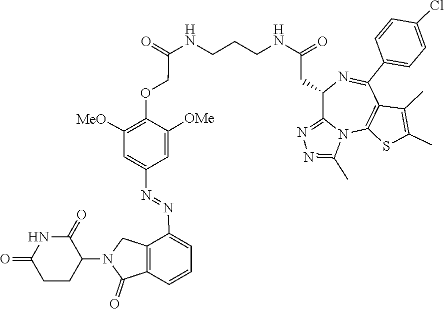 [SMILES: COc1cc(/N=N/c2cccc3c2CN(C2CCC(=O)NC2=O)C3=O)cc(OC)c1OCC(=O)NCCCNC(=O)C[C@@H]1N=C(c2ccc(Cl)cc2)c2c(sc(C)c2C)-n2c(C)nnc21]

Into a round bottom flask with dry (+)-JQ1 free acid (7.2 mg, 0.018 mmol, 1 eq.) were added S9 (23.5 mg, 0.036 mmol, 2 eq.) and HATU (11.7 mg, 0.036 mmol, 1.5 eq.) under nitrogen. The reaction was dissolved in dry DMF (1 mL). After addition of i-Pr₂NEt (17 mg, 0.13 mmol, 7.2 eq., 0.023 mL) the reaction was stirred for 14 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-2 (15.1 mg, 0.016 mmol, 91%) as a yellow solid. R_{f}=0.31 [CH₂Cl₂:MeOH, 19:1]. ¹H NMR (400 MHz, Chloroform-d) δ=8.52 (s, 1H), 8.18 (d, J=7.8 Hz, 1H), 7.99 (d, J=7.5 Hz, 1H), 7.80 (t, J=5.8 Hz, 1H), 7.69 (t, J=7.7 Hz, 1H), 7.39 (d, J=8.4 Hz, 2H), 7.31 (d, J=8.3 Hz, 2H), 7.21 (d, J=2.9 Hz, 2H), 7.07 (s, 1H), 5.28-5.19 (m, 1H), 4.85 (d, J=18.0 Hz, 1H), 4.71 (dd, J=18.1, 3.2 Hz, 1H), 4.63 (d, J=6.8 Hz, 1H), 4.60 (s, 2H), 3.97 (s, 6H), 3.55 (dd, J=14.4, 7.6 Hz, 1H), 3.42 (q, J=6.4 Hz, 2H), 3.35 (q, J=7.6, 6.9 Hz, 3H), 2.95-2.77 (m, 2H), 2.64 (s, 3H), 2.46 (dt, J=12.9, 4.5 Hz, 1H), 2.38 (s, 3H), 2.28-2.18 (m, 1H), 1.79 (p, J=6.4 Hz, 2H), 1.66 (s, 3H) ppm. ¹³C NMR (100 MHz, CDCl₃) δ=171.37, 170.84, 169.95, 169.72, 168.57, 164.10, 155.77, 152.78, 150.07, 148.99, 146.90, 139.86, 136.92, 136.69, 134.15, 133.46, 132.26, 131.02, 130.98, 130.58, 130.02, 129.96, 129.57, 128.83, 126.29, 100.64, 72.79, 56.55, 54.55, 52.12, 48.37, 39.39, 36.91, 36.45, 31.73, 29.76, 23.56, 14.52, 13.22, 11.94 ppm. HRMS (ESI): calcd. for C₄₅H₄₅CIN₁₀NaO₈S⁺: 943.2723 m/z [M+Na]⁺; found: 943.2738 m/z [M+Na]⁺. LCMS (ESI): tᵣₑₜ=3.71 min. 920 m/z [M−H]⁻.

### 2-((S)-4-(4-chlorophenyl)-2,3,9-trimethyl-6H-thieno[3,2-f][1,2,4]triazolo[4,3-a][1,4]diazepin-6-yl)-N-(4-(2-(4-((E)-(2-(2,6-dioxopiperidin-3-yl)-1-oxoisoindolin-4-yl)diazenyl)-2,6-dimethoxyphenoxy)acetamido)butyl)acetamide (PHOTAC-I-3)

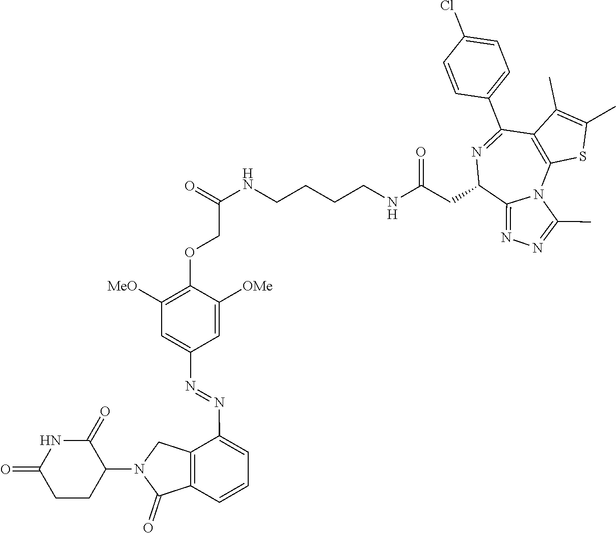 [SMILES: COc1cc(/N=N/c2cccc3c2CN(C2CCC(=O)NC2=O)C3=O)cc(OC)c1OCC(=O)NCCCCNC(=O)C[C@@H]1N=C(c2ccc(Cl)cc2)c2c(sc(C)c2C)-n2c(C)nnc21]

Into a round bottom flask with dry (+)-JQ1 free acid (7.2 mg, 0.018 mmol, 1 eq.) were added 4 (26.6 mg, 0.04 mmol, 2 eq.) and HATU (11.7 mg, 0.036 mmol, 1.8 eq.) under nitrogen atmosphere. The solids were dissolved in dry DMF (1 mL). After addition of i-Pr₂NEt (18.6 mg, 0.144 mmol, 7.2 eq., 0.025 mL) the reaction was stirred for 16 h at room temperature. The mixture was then diluted with EtOAc (20 mL), separated against 5% LiCl (20 mL), extracted with EtOAc (2×20 mL), washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-3 (15.6 mg, 0.017 mmol, 85%) as a yellow solid. R_{f}=0.30 [CH₂Cl₂:MeOH, 19:1]. ¹H NMR (400 MHz, Chloroform-d) δ=8.80 (d, J=67.2 Hz, 1H), 8.15 (t, J=8.7 Hz, 1H), 8.00-7.92 (m, 1H), 7.75 (d, J=4.9 Hz, 1H), 7.67 (q, J=7.2 Hz, 1H), 7.38 (d, J=8.2 Hz, 2H), 7.31 (d, J=8.2 Hz, 2H), 7.21 (d, J=8.8 Hz, 2H), 6.85 (dt, J=10.4, 5.2 Hz, 1H), 5.21 (dd, J=9.2, 3.9 Hz, 1H), 4.84 (d, J=18.0 Hz, 1H), 4.69 (d, J=18.0 Hz, 1H), 4.62 (d, J=6.8 Hz, 3H), 3.97 (d, J=2.5 Hz, 6H), 3.53 (dd, J=12.5, 7.5 Hz, 1H), 3.42-3.23 (m, 5H), 2.90-2.72 (m, 2H), 2.64 (d, J=5.4 Hz, 3H), 2.50-2.41 (m, 1H), 2.38 (s, 3H), 2.24-2.13 (m, 1H), 1.65 (s, 4H), 1.61 (s, 3H) ppm. ¹³C NMR (100 MHz, CDCl₃) δ=171.44, 170.62, 169.90, 169.64, 168.53, 164.21, 155.74, 152.69, 150.12, 148.91, 146.83, 139.88, 136.96, 136.68, 134.18, 133.45, 132.24, 131.04, 131.01, 130.58, 130.01, 129.94, 129.52, 128.85, 126.20, 100.64, 72.85, 56.54, 54.59, 52.10, 48.35, 39.46, 39.37, 38.78, 31.71, 27.17, 27.04, 23.54, 14.51, 13.21, 11.91 ppm. HRMS (ESI): calcd. for C₄₆H₄₈CIN₁₀O₈S⁺: 935.3060 m/z [M+H]⁺; found: 973.2597 m/z [M+H]⁺. LCMS (ESI): tᵣₑₜ=3.55 min (Z). 935 m/z [M+H]⁺. tᵣₑₜ=3.74 min (F). 935 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(5-(2-(4-((E)-(2-(2,6-dioxopiperidin-3-yl)-1-oxoisoindolin-4-yl)diazenyl)-2,6-dimethoxyphenoxy)acetamido)pentyl)acetamide (PHOTAC-I-4)

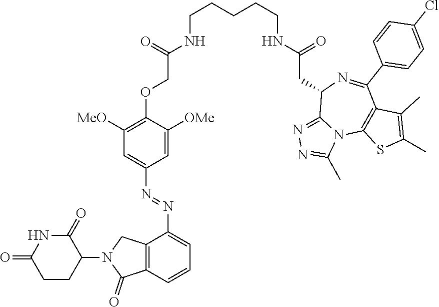 [SMILES: COc1cc(/N=N/c2cccc3c2CN(C2CCC(=O)NC2=O)C3=O)cc(OC)c1OCC(=O)NCCCCCNC(=O)C[C@@H]1N=C(c2ccc(Cl)cc2)c2c(sc(C)c2C)-n2c(C)nnc21]

Into a round bottom flask with dry (+)-JQ1 free acid (7.2 mg, 0.018 mmol, 1 eq.) were added S10 (24.4 mg, 0.036 mmol, 2 eq.) and HATU (10.5 mg, 0.032 mmol, 1.8 eq.) under nitrogen atmosphere. The solids were dissolved in dry DMF (1 mL). After addition of i-Pr₂NEt (16.7 mg, 0.129 mmol, 7.2 eq., 0.023 mL) the reaction was stirred for 15 h at room temperature. The mixture was then diluted with EtOAc (20 mL), separated against 5% LiCl (20 mL), extracted with EtOAc (2×20 mL), washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-4 (15.4 mg, 0.016 mmol, 89%) as a yellow solid. R_{f}=0.31 [CH₂Cl₂:MeOH, 19:1]. ¹H NMR (400 MHz, Chloroform-d) δ=8.66 (d, J=14.5 Hz, 1H), 8.18 (dd, J=7.6, 3.5 Hz, 1H), 7.99 (d, J=7.5 Hz, 1H), 7.69 (t, J=7.2 Hz, 2H), 7.39 (d, J=8.1 Hz, 2H), 7.32 (d, J=7.7 Hz, 2H), 7.22 (d, J=5.8 Hz, 2H), 6.79-6.68 (m, 1H), 5.23 (dt, J=12.2, 5.2 Hz, 1H), 4.87 (s, 1H), 4.74 (s, 1H), 4.60 (d, J=11.6 Hz, 3H), 3.98 (s, 6H), 3.55 (dt, J=10.9, 5.4 Hz, 1H), 3.31 (dt, J=14.7, 5.9 Hz, 5H), 2.95-2.77 (m, 2H), 2.65 (s, 3H), 2.46 (d, J=12.7 Hz, 1H), 2.39 (s, 3H), 2.31-2.14 (m, 1H), 1.66 (s, 3H), 1.57 (q, J=6.9 Hz, 4H), 1.48-1.34 (m, 2H) ppm. ¹³C NMR (100 MHz, CDCl₃) δ=171.39, 170.62, 169.82, 169.59, 168.57, 164.15, 155.73, 152.71, 150.09, 148.96, 146.90, 139.89, 136.97, 136.67, 134.17, 133.45, 132.23, 131.05, 131.04, 130.59, 130.09, 129.94, 129.56, 128.86, 126.29, 100.65, 72.86, 56.53, 54.65, 52.11, 48.35, 39.58, 39.51, 38.92, 31.73, 29.37, 29.26, 24.30, 23.57, 14.51, 13.23, 11.92 ppm. HRMS (ESI): calcd. for C₄₇H₄₉CIKN₁₀O₈S⁺: 987.2776 m/z [M+K]⁺; found: 987.2755 m/z [M+K]⁺. LCMS (ESI): tᵣₑₜ=3.83 min. 949 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(6-(2-(4-((E)-(2-(2,6-dioxopiperidin-3-yl)-1-oxoisoindolin-4-yl)diazenyl)-2,6-dimethoxyphenoxy)acetamido)hexyl)acetamide (PHOTAC-I-5)

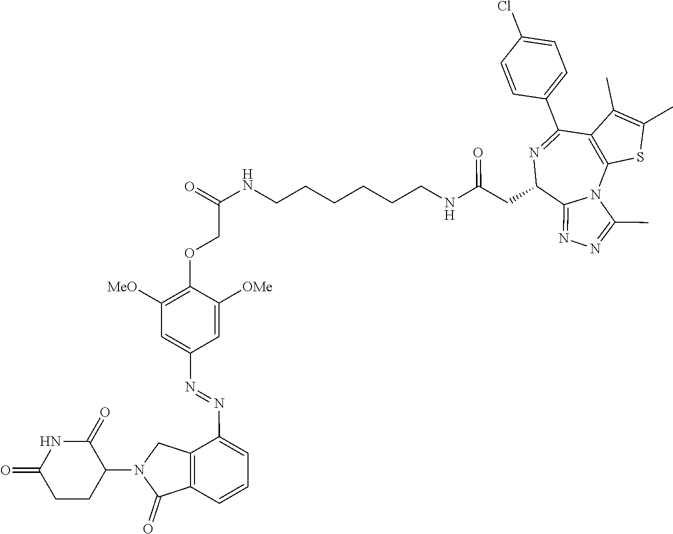 [SMILES: COc1cc(/N=N/c2cccc3c2CN(C2CCC(=O)NC2=O)C3=O)cc(OC)c1OCC(=O)NCCCCCCNC(=O)C[C@@H]1N=C(c2ccc(Cl)cc2)c2c(sc(C)c2C)-n2c(C)nnc21]

Into a round bottom flask with dry (+)-JQ1 free acid (7.6 mg, 0.019 mmol, 1 eq.) were added S11 (26.3 mg, 0.038 mmol, 2 eq.) and HATU (11.1 mg, 0.034 mmol, 1.8 eq.) under nitrogen atmosphere. The solids were dissolved in dry DMF (1 mL). After addition of i-Pr₂NEt (17.6 mg, 0.137 mmol, 7.2 eq., 0.024 mL) the reaction was stirred for 16 h at room temperature. The mixture was then diluted with EtOAc (20 mL), separated against 5% LiCl (20 mL), extracted with EtOAc (2×20 mL), washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-5 (16.4 mg, 0.017 mmol, 90%) as a yellow solid. R_{f}=0.31 [CH₂Cl₂:MeOH, 19:1]. ¹H NMR (400 MHz, Chloroform-d) δ=8.78 (d, J=29.2 Hz, 1H), 8.18 (d, J=7.6 Hz, 1H), 7.99 (d, J=7.2 Hz, 1H), 7.69 (t, J=7.5 Hz, 2H), 7.39 (d, J=8.3 Hz, 2H), 7.31 (d, J=8.3 Hz, 2H), 7.26 (s, 1H), 7.22 (s, 1H), 6.72-6.60 (m, 1H), 5.22 (td, J=13.8, 4.9 Hz, 1H), 4.93-4.79 (m, 1H), 4.70 (dd, J=18.0, 5.8 Hz, 1H), 4.62 (s, 3H), 3.98 (d, J=3.6 Hz, 6H), 3.54 (td, J=7.7, 3.8 Hz, 1H), 3.38-3.21 (m, 5H), 2.96-2.74 (m, 2H), 2.64 (d, J=10.7 Hz, 3H), 2.52-2.42 (m, 1H), 2.38 (s, 3H), 2.22 (s, 1H), 1.65 (s, 3H), 1.60-1.48 (m, 4H), 1.44-1.31 (m, 4H) ppm. ¹³C NMR (100 MHz, CDCl₃) δ=171.42, 170.53, 169.87, 169.53, 168.56, 164.29, 155.70, 152.71, 150.10, 148.94, 146.88, 139.91, 136.98, 136.67, 134.29, 133.46, 132.27, 131.04, 131.02, 130.58, 129.96, 129.75, 129.56, 128.85, 126.29, 100.68, 72.91, 56.51, 54.65, 52.03, 48.24, 39.64, 39.51, 38.98, 31.74, 29.57, 29.55, 26.74, 26.70, 23.58, 14.50, 13.22, 11.88 ppm. HRMS (ESI): calcd. for C₄₈H₅₁CIN₁₀NaO₈S⁺: 985.3193 m/z [M+Na]⁺; found: 985.3234 m/z [M+Na]⁺. LCMS (ESI): tᵣₑₜ=3.93 min. 963 m/z [M+H]⁺.

### (E)-3-(4-(4-hydroxyphenyl)diazenyl)-1-oxoisoindolin-2-yl)piperidine-2,6-dione (S12)

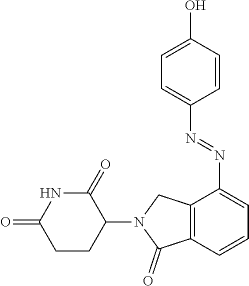 [SMILES: O=C1CCC(N2Cc3c(/N=N/c4ccc(O)cc4)cccc3C2=O)C(=O)N1]

Lenalidomide (500 mg, 1.93 mmol, 1.0 eq.) was dissolved in 1 M HCl (50 mL). Concentrated aqueous HBF₄ (1 mL) was added to the mixture. After complete dissolving of the starting material, 2 M NaNO₂ (1.06 mL) was added to the solution at 0° C. After stirring for 1 h the solution was added dropwise into a mixture of Phenol (217.9 mg, 2.315 mmol, 1.2 eq., 0.204 mL) in H₂O (50 mL), MeOH (20 mL), NaHCO₃ (4.000 g, 47.62 mmol, 24.7 eq.) and Na₂CO₃ (5.000 g, 47.18 mmol, 24.5 eq.) at 0° C. Upon addition the solution turned from white to orange and stirred for additional 1 h at 0° C. The reaction was extracted with EtOAc (7×100 mL) and washed once with brine (1×100 mL). The organic phase was dried over Na₂SO₄ and then concentrated under reduced pressure. S12 (605.2 mg, 1.661 mmol, 86%) was obtained as a yellow solid. R_{f}=0.26 [CH₂Cl₂:MeOH, 19:1]. ¹H NMR (400 MHz, DMSO-d₆) δ=11.01 (s, 1H), 10.42 (s, 1H), 8.13 (d, J=7.5 Hz, 1H), 7.97-7.68 (m, 4H), 6.96 (d, J=8.1 Hz, 2H), 5.23-5.07 (m, 1H), 4.84-4.57 (m, 2H), 2.94 (t, J=12.9 Hz, 1H), 2.67-2.54 (m, 2H), 2.10-1.95 (m, 1H) ppm. ¹³C NMR (100 MHz, DMSO) δ=172.92, 171.02, 167.28, 161.61, 146.70, 145.40, 134.16, 133.69, 129.50, 128.11, 125.16, 124.52, 115.97, 51.65, 48.21, 31.25, 22.34 ppm. HRMS (ESI): calcd. for C₁₉H₁₇N₄O₄⁺: 365.1244 m/z [M+H]⁺; found: 365.1257 m/z [M+H]⁺. LCMS (ESI): tᵣₑₜ=2.94 min. 365 m/z [M+H]⁺.

### (E)-3-(4-((4-(2-(tert-butoxy)-2-oxoethoxy)phenyl)diazenyl)-1-oxoisoindolin-2-yl)-2,6-dioxopiperidin-1-ium (S13)

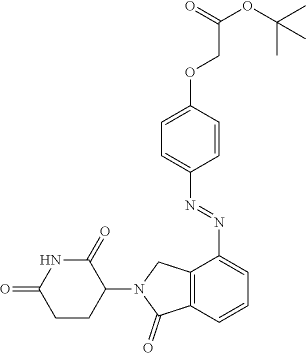 [SMILES: CC(C)(C)OC(=O)COc1ccc(/N=N/c2cccc3c2CN(C2CCC(=O)NC2=O)C3=O)cc1]

To a solution of tert-butyl bromoacetate (267.6 mg, 1.372 mmol, 1 eq., 0.20 mL) was added S12 (500 mg, 1.372 mmol, 1 eq.) dissolved in dry DMF (13 mL). After addition of K2CO₃ (246.6 mg, 1.784 mmol, 1.3 eq.) the reaction was stirred for 6.5 h at room temperature. Upon addition the solution turned from orange to dark red. After 6.5 hours of stirring, the mixture was diluted with EtOAc (100 mL), separated against NaHCO₃ (50 mL), extracted with EtOAc (3×50 mL) and washed with brine (4×50 mL). The reaction was concentrated under reduced pressure. Purification of the resulting crude product by flash column chromatography (Hx/Ea gradient, 20→100% Ea) gave S13 (397.7 mg, 0831 mmol, 61%) as a yellow solid. R_{f}=0.44 [Hx:EA, 1:4]. ¹H NMR (400 MHz, DMSO-d₆) δ=11.01 (s, 1H), 8.18 (d, J=7.7 Hz, 1H), 8.03-7.94 (m, 2H), 7.89 (d, J=7.4 Hz, 1H), 7.78 (t, J=7.7 Hz, 1H), 7.17-7.08 (m, 2H), 5.17 (dd, J=13.5, 5.0 Hz, 1H), 4.79 (m, 3H), 4.67 (d, J=18.9 Hz, 1H), 2.92 (d, J=12.7 Hz, 1H), 2.58 (dd, J=23.6, 14.7 Hz, 2H), 2.02 (dd, J=16.0, 9.6 Hz, 1H), 1.44 (s, 9H) ppm. ¹³C NMR (100 MHz, DMSO) δ=172.92, 171.01, 167.42, 167.22, 160.89, 146.64, 146.60, 134.26, 133.75, 129.58, 128.58, 124.99, 124.73, 115.18, 81.70, 65.20, 51.66, 48.26, 31.25, 27.70, 22.32 ppm. HRMS (APCI): calcd. for C₂₅H₂₇N₄O₆⁺: 479.1925 m/z [M+H]⁺; found: 479.1928 m/z [M+H]⁺. LCMS (ESI): tᵣₑₜ=4.45 min. 479 m/z [M+H]⁺.

### (E)-3-(4-((4-(carboxymethoxy)phenyl)diazenyl)-1-oxoisoindolin-2-yl)-2,6-dioxopiperidin-1-ium (S14)

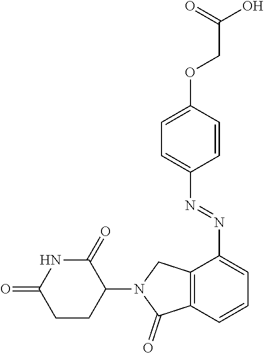 [SMILES: O=C(O)COc1ccc(/N=N/c2cccc3c2CN(C2CCC(=O)NC2=O)C3=O)cc1]

S13 (331.1 mg, 0.692 mmol, 1 eq.) was dissolved in CH₂Cl₂:TFA (1:1; 4 mL each). Upon TFA addition (4 mL) the solution turned from yellow to dark red. After 4 hours the reaction was concentrated under reduced pressure, turning from red to an orange solid. The reaction was triturated with Et₂O and dried under high vacuum for 24 h. S14 (340.5 mg, 0.635 mmol, 92%) was obtained in form of a yellow solid. R_{f}=0.10 [CH₂Cl₂:MeOH, 9:1]. ¹H NMR (400 MHz, DMSO-d₆) δ=11.01 (s, 1H), 8.22-8.13 (m, 1H), 8.02-7.94 (m, 2H), 7.88 (d, J=7.5 Hz, 1H), 7.78 (t, J=7.8 Hz, 1H), 7.20-7.09 (m, 2H), 5.16 (dd, J=13.4, 5.0 Hz, 1H), 4.80 (m, 3H), 4.67 (d, J=19.2 Hz, 1H), 2.94 (ddd, J=18.4, 13.9, 5.3 Hz, 1H), 2.69-2.54 (m, 2H), 2.08-1.98 (m, 1H) ppm. ¹³C NMR (100 MHz, DMSO) δ=172.93, 171.01, 169.76, 167.23, 161.01, 146.62, 146.62, 134.32, 133.75, 129.58, 128.43, 124.97, 124.74, 115.19, 64.76, 51.68, 48.24, 31.25, 22.32 ppm. HRMS (APCI): calcd. for C₂₁H₁₉N₄O₆⁺: 423.1299 m/z [M+H]⁺; found: 423.1284 m/z [M+H]⁺. LCMS (ESI): tᵣₑₜ=3.01 min. 423 m/z [M+H]⁺.

### tert-butyl (E)-(2-(2-(4-((2-(2,6-dioxopiperidin-3-yl)-1-oxoisoindolin-4-yl)diazenyl)phenoxy)acetamido)ethyl)carbamate (S15)

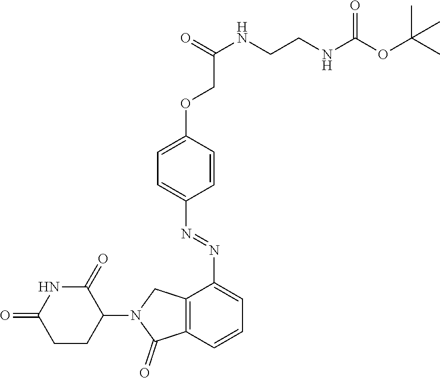 [SMILES: CC(C)(C)OC(=O)NCCNC(=O)COc1ccc(/N=N/c2cccc3c2CN(C2CCC(=O)NC2=O)C3=O)cc1]

S14 (50.0 mg, 0.093 mmol, 1.0 eq.) and HATU (45.3 mg, 0.140 mmol, 1.5 eq.) were dissolved in dry DMF (4.5 mL) at room temperature under nitrogen atmosphere. After 5 minutes of stirring N-Boc-1,4-diaminoethane (80.7 mg, 0.373 mmol, 4 eq.) and i-Pr₂NEt (48.2 mg, 0.373 mmol, 4 eq., 66 μL) were added to the mixture and stirred for further 12 hours. The reaction was diluted with EtOAc (20 mL), separated against H₂O: sat. NaCl (20 mL), extracted with EtOAc (3×20 mL) and washed with brine (4×20 mL). The combined organic phase was dried over Na₂SO₄ and concentrated under reduced pressure. S15 (57.2 mg, 0.092 mmol, 99%) was obtained as an orange solid. R_{f}=0.38 [CH₂Cl₂:MeOH, 19:1]. ¹H NMR (400 MHz, DMSO-d₆) δ=10.99 (s, 1H), 8.20 (dd, J=16.2, 6.6 Hz, 2H), 7.99 (d, J=8.4 Hz, 2H), 7.89 (d, J=7.4 Hz, 1H), 7.78 (t, J=7.6 Hz, 1H), 7.18 (d, J=8.6 Hz, 2H), 6.87 (q, J=5.7, 4.7 Hz, 1H), 5.17 (dd, J=13.2, 5.1 Hz, 1H), 4.80 (d, J=19.1 Hz, 1H), 4.67 (d, J=19.3 Hz, 1H), 4.61 (s, 2H), 3.18 (q, J=6.3 Hz, 2H), 3.04 (q, J=6.3 Hz, 2H), 2.96-2.88 (m, 1H), 2.60 (dd, J=16.2, 12.6 Hz, 2H), 2.09-1.98 (m, 1H), 1.37 (s, 9H) ppm. ¹³C NMR (100 MHz, DMSO) δ=172.91, 171.00, 167.26, 167.21, 160.88, 155.74, 146.68, 146.59, 134.27, 133.74, 129.59, 128.52, 124.99, 124.75, 115.40, 77.73, 67.11, 51.66, 48.24, 38.72, 31.25, 28.22, 22.34, 22.30 ppm. HRMS (APCI): calcd. for C₂₈H₃₃N₆O₇⁺: 565.2405 m/z [M+H]⁺; found: 565.2381 m/z [M+H]⁺. LCMS (ESI): tᵣₑₜ=2.96 min (Z). 563 m/z [M−H]⁻. tᵣₑₜ=3.38 min (E). 563 m/z [M−H]⁻.

### tert-butyl (E)-(4-(2-(4-((2-(2,6-dioxopiperidin-3-yl)-1-oxoisoindolin-4-yl)diazenyl)phenoxy)acetamido)butyl)carbamate (S16)

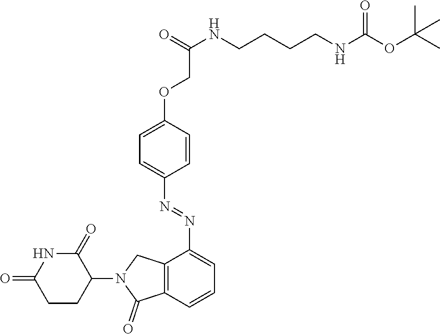 [SMILES: CC(C)(C)OC(=O)NCCCCNC(=O)COc1ccc(/N=N/c2cccc3c2CN(C2CCC(=O)NC2=O)C3=O)cc1]

S14 (50.0 mg, 0.093 mmol, 1.0 eq.) and HATU (46.0 mg, 0.142 mmol, 1.5 eq.) were dissolved in dry DMF (4.5 mL) at room temperature. After 5 minutes of stirring N-Boc-1,4-diaminobutane (70.2 mg, 0.373 mmol, 4 eq.) and i-Pr₂NEt (48.2 mg, 0.373 mmol, 4 eq., 66 μL) were added to the mixture and stirred for additional 12 h at room temperature. The reaction was diluted with EtOAc (20 mL), separated against 5% LiCl (20 mL), extracted with EtOAc (3×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave S16 (50.9 mg, 0.086 mmol, 92%) as an orange solid. R_{f}=0.38 [CH₂Cl₂:MeOH, 19:1]. ¹H NMR (400 MHz, Chloroform-d) δ=8.18 (d, J=7.9 Hz, 1H), 8.12 (s, 1H), 7.99 (d, J=7.5 Hz, 1H), 7.95-7.90 (m, 2H), 7.70 (t, J=7.7 Hz, 1H), 7.05 (d, J=8.9 Hz, 2H), 6.66 (s, 1H), 5.26 (dd, J=13.3, 5.1 Hz, 1H), 4.85 (d, J=17.9 Hz, 1H), 4.74 (d, J=18.0 Hz, 1H), 4.57 (s, 2H), 3.39 (q, J=6.6 Hz, 2H), 3.14 (q, J=6.5 Hz, 2H), 3.01-2.79 (m, 2H), 2.46 (qd, J=13.1, 5.0 Hz, 1H), 2.26 (dtd, J=12.9, 5.0, 2.5 Hz, 1H), 1.62-1.48 (m, 4H), 1.43 (s, 9H) ppm. ¹³C NMR (100 MHz, CDCl₃) δ=171.08, 169.55, 168.67, 167.59, 160.02, 156.19, 147.95, 147.09, 134.17, 133.37, 129.70, 129.57, 126.02, 125.12, 115.24, 79.43, 67.62, 52.11, 48.35, 40.26, 38.94, 31.74, 28.56, 27.69, 26.93, 23.61 ppm. HRMS (APCI): calcd. for C₃₀H₃₇N₆O₇⁺: 593.2718 m/z [M+H]⁺; found: 593.2700 m/z [M+H]⁺. LCMS (ESI): tᵣₑₜ=3.17 min (Z). 591 m/z [M−H]⁻. tᵣₑₜ=3.53 min (E). 591 m/z [M−H]⁻.

### tert-butyl (E)-(6-(2-(4-((2-(2,6-dioxopiperidin-3-yl)-1-oxoisoindolin-4-yl)diazenyl)phenoxy)acetamido)hexyl)carbamate (S17)

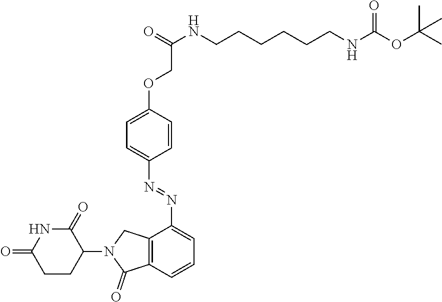 [SMILES: CC(C)(C)OC(=O)NCCCCCCNC(=O)COc1ccc(/N=N/c2cccc3c2CN(C2CCC(=O)NC2=O)C3=O)cc1]

S14 (50.0 mg, 0.093 mmol, 1.0 eq.) and HATU (45.3 mg, 0.140 mmol, 1.5 eq.) were dissolved in dry DMF (4.5 mL) at room temperature. After 5 minutes of stirring N-Boc-1,4-diaminohexane (80.7 mg, 0.373 mmol, 4 eq.) and i-Pr₂NEt (48.2 mg, 0.373 mmol, 4 eq., 66 μL) were added to the mixture and stirred for additional 12 h at room temperature. The reaction was diluted with EtOAc (20 mL), separated against 5% LiCl (20 mL), extracted with EtOAc (3×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave S17 (57.2 mg, 0.092 mmol, 99%) as an orange solid. R_{f}=0.34 [CH₂Cl₂:MeOH, 19:1]. ¹H NMR (400 MHz, Chloroform-d) δ=8.19 (d, J=7.8 Hz, 1H), 8.04 (s, 1H), 7.99 (d, J=7.5 Hz, 1H), 7.94 (d, J=8.9 Hz, 2H), 7.70 (t, J=7.6 Hz, 1H), 7.06 (d, J=8.9 Hz, 2H), 6.59 (s, 1H), 5.26 (dd, J=13.3, 5.1 Hz, 1H), 4.86 (d, J=17.9 Hz, 1H), 4.74 (d, J=18.0 Hz, 1H), 4.59 (s, 2H), 3.36 (q, J=6.8 Hz, 2H), 3.09 (s, 2H), 3.00-2.79 (m, 2H), 2.46 (dd, J=13.1, 5.0 Hz, 1H), 2.27 (ddd, J=13.9, 7.0, 3.6 Hz, 1H), 1.55 (d, J=7.0 Hz, 2H), 1.50-1.43 (m, 2H), 1.44 (s, 9H), 1.34 (d, J=6.0 Hz, 4H) ppm. ¹³C NMR (100 MHz, CDCl₃) δ=170.90, 169.91, 169.40, 168.53, 167.38, 159.95, 147.82, 146.98, 134.03, 133.25, 129.63, 129.45, 125.90, 125.00, 115.11, 79.16, 67.54, 51.96, 48.19, 40.29, 38.93, 31.61, 30.00, 29.47, 28.44, 26.34, 26.23, 23.50 ppm. HRMS (ESI): calcd. for C₃₂H₄₁N₆O₇⁺: 621.3031 m/z [M+H]⁺; found: 621.3017 m/z [M+H]⁺. LCMS (APCI): tᵣₑₜ=3.44 min. (Z) 619 m/z [M−H]⁻. tᵣₑₜ=3.80 min. (E). 619 m/z [M−H]⁻.

### (E)-N-(2-aminoethyl)-2-(4-((2-(2,6-dioxopiperidin-3-yl)-1-oxoisoindolin-4-yl)diazenyl)phenoxy)acetamide (S18)

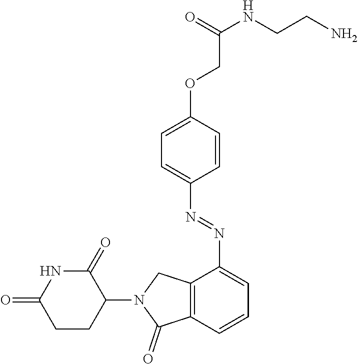 [SMILES: NCCNC(=O)COc1ccc(/N=N/c2cccc3c2CN(C2CCC(=O)NC2=O)C3=O)cc1]

S15 (35.0 mg, 0.062 mmol, 1 eq.) was dissolved in TFA/CH₂Cl₂ (1:1; 1 mL:1 mL) and stirred for 2 h at room temperature. The reaction was diluted with CH₂Cl₂ and concentrated under reduced pressure. The reaction was triturated with Et₂O and dried on high vacuum overnight. S18 (34.5 mg, 0.06 mmol, 96%) was obtained as trifluoroacetate in form of a yellow solid with traces of residual TFA. R_{f}=0.13 [CH₂Cl₂₊₁% TEA:MeOH, 5:1]. ¹H NMR (400 MHz, DMSO-d₆) δ=11.02 (s, 1H), 8.36 (t, J=6.0 Hz, 1H), 8.18 (d, J=7.8 Hz, 1H), 8.01 (d, J=8.4 Hz, 2H), 7.90 (d, J=7.4 Hz, 1H), 7.83-7.71 (m, 4H), 7.21 (d, J=8.4 Hz, 2H), 5.19 (d, J=13.4 Hz, 1H), 4.80 (d, J=19.1 Hz, 1H), 4.67 (d, J=16.9 Hz, 3H), 2.99-2.87 (m, 3H), 2.69-2.51 (m, 1H), 2.11-1.98 (m, 1H) ppm. ¹³C NMR (100 MHz, DMSO) δ=172.93, 171.02, 168.11, 167.21, 160.73, 146.76, 146.58, 134.27, 133.76, 129.62, 128.57, 125.07, 124.80, 115.44, 67.14, 51.65, 48.22, 38.72, 36.20, 31.25, 22.35 ppm. HRMS (APCI): calcd. for C₂₃H₂₅N₆O₅⁺: 465.1881 m/z [M+H]⁺; found: 465.1887 m/z [M+H]⁺. LCMS (ESI): tᵣₑₜ=2.00 min (Z). 465 m/z [M+H]⁺. tᵣₑₜ=2.32 min (E). 465 m/z [M+H]⁺.

### N-(4-aminobutyl)-2-(4-((2-(2,6-dioxopiperidin-3-yl)-1-oxoisoindolin-4-yl)diazenyl)phenoxy)acetamide (S19)

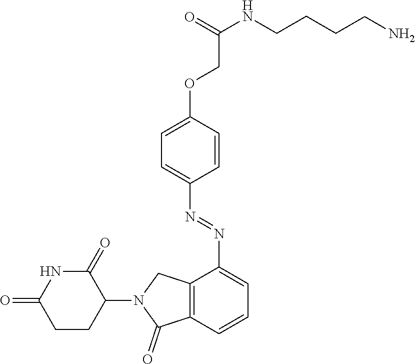 [SMILES: NCCCCNC(=O)COc1ccc(/N=N/c2cccc3c2CN(C2CCC(=O)NC2=O)C3=O)cc1]

S16 (40.0 mg, 0.067 mmol, 1 eq.) was dissolved in TFA/CH₂Cl₂ (1:1; 1 mL:1 mL) and stirred for 2 h at room temperature. The reaction was diluted with CH₂Cl₂ and concentrated under reduced pressure. The reaction was triturated with Et₂O and dried on high vacuum overnight. S19 (40.5 mg, 0.067 mmol, 99%) was obtained as trifluoroacetate in form of a yellow solid with traces of residual TFA. R_{f}=0.13 [CH₂Cl₂₊₁% TEA:MeOH, 5:1]. ¹H NMR (400 MHz, DMSO-d₆) δ=11.02 (s, 1H), 8.26 (t, J=5.9 Hz, 1H), 8.18 (d, J=7.8 Hz, 1H), 8.03-7.97 (m, 2H), 7.89 (d, J=7.5 Hz, 1H), 7.78 (t, J=7.7 Hz, 1H), 7.67 (s, 2H), 7.22-7.14 (m, 2H), 5.18 (dd, J=13.1, 5.0 Hz, 1H), 4.80 (d, J=19.1 Hz, 1H), 4.67 (d, J=19.3 Hz, 1H), 4.62 (s, 2H), 3.17 (q, J=6.2 Hz, 2H), 2.94 (dd, J=10.9, 6.4 Hz, 1H), 2.80 (q, J=6.4 Hz, 2H), 2.65-2.54 (m, 2H), 2.05 (d, J=6.1 Hz, 1H), 1.51 (hept, J=6.0, 5.4 Hz, 4H) ppm. ¹³C NMR (100 MHz, DMSO) δ=172.94, 171.03, 167.22, 167.09, 160.89, 146.71, 146.59, 134.28, 133.76, 129.62, 128.53, 125.04, 124.78, 115.41, 67.18, 51.65, 48.22, 38.60, 37.67, 31.25, 26.18, 24.48, 22.34 ppm. HRMS (APCI): calcd. for C₂₅H₂₉N₆O₅⁺: 493.2194 m/z [M+H]⁺; found: 493.2177 m/z [M+H]⁺. LCMS (ESI): tᵣₑₜ=2.21 min (Z). 493 m/z [M+H]⁺. tᵣₑₜ=2.44 min (F). 493 m/z [M+H]⁺.

### Tert-butyl-(6-(2-(4-((2-(2,6-dioxopiperidin-3-yl)-1-oxoisoindolin-4-yl)diazenyl)phenoxy)-acetamido)hexyl)carbamate (S20)

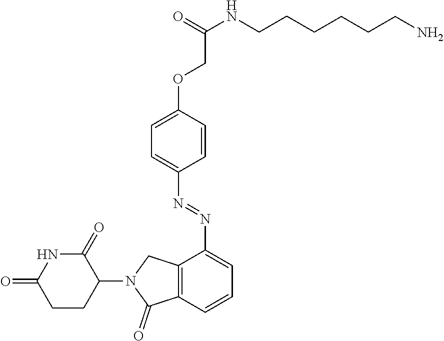 [SMILES: NCCCCCCNC(=O)COc1ccc(/N=N/c2cccc3c2CN(C2CCC(=O)NC2=O)C3=O)cc1]

S17 (44.5 mg, 0.072 mmol, 1 eq.) was dissolved in TFA/CH₂Cl₂ (1:1; 2 mL:2 mL) and stirred for 2 h at room temperature. The reaction was diluted with CH₂Cl₂ and concentrated under reduced pressure. The reaction was triturated with Et₂O and dried on high vacuum overnight. S20 (45.6 mg, 0.072 mmol, >99%) was obtained as trifluoroacetate in form of a yellow solid with traces of residual TFA. R_{f}=0.16 [CH₂Cl₂₊₁% TEA:MeOH, 5:1]. ¹H NMR (400 MHz, DMSO-d₆) δ=11.02 (s, 1H), 8.24-8.14 (m, 2H), 7.99 (d, J=7.2 Hz, 2H), 7.89 (d, J=7.4 Hz, 1H), 7.78 (t, J=7.7 Hz, 1H), 7.66 (s, 2H), 7.17 (d, J=7.6 Hz, 2H), 5.18 (dd, J=13.2, 5.0 Hz, 1H), 4.80 (d, J=19.1 Hz, 1H), 4.67 (d, J=19.4 Hz, 1H), 4.61 (s, 2H), 3.14 (q, J=6.7 Hz, 2H), 2.94 (ddd, J=21.8, 11.5, 4.6 Hz, 1H), 2.76 (h, J=6.2 Hz, 2H), 2.67-2.53 (m, 2H), 2.04 (dt, J=11.8, 4.5 Hz, 1H), 1.57-1.39 (m, 4H), 1.29 (tq, J=11.9, 7.0 Hz, 4H) ppm. ¹³C NMR (100 MHz, DMSO) δ=172.94, 171.03, 167.22, 166.92, 160.95, 146.69, 146.59, 134.29, 133.76, 129.61, 128.51, 125.03, 124.75, 115.40, 67.19, 51.66, 48.23, 38.78, 38.19, 31.25, 28.93, 26.95, 25.84, 25.46, 22.34 ppm. HRMS (APCI): calcd. for C₂₇H₃₃N₆O₅⁺: 521.2507 m/z [M+H]⁺; found: 521.2498 m/z [M+H]⁺. LCMS (ESI): tᵣₑₜ=2.37 min (cis). 521 m/z [M+H]⁺. tᵣₑₜ=2.80 min (trans). 521 m/z [M+H]⁺.

### 2-((R)-4-(4-chlorophenyl)-2,3,9-trimethyl-6H-thieno[3,2-f][1,2,4]triazolo[4,3-a][1,4]diazepin-6-yl)-N-(2-(2-(4-((E)-(2-(2,6-dioxopiperidin-3-yl)-1-oxoisoindolin-4-yl)diazenyl)phenoxy)acetamido)ethyl)acetamide (PHOTAC-I-6)

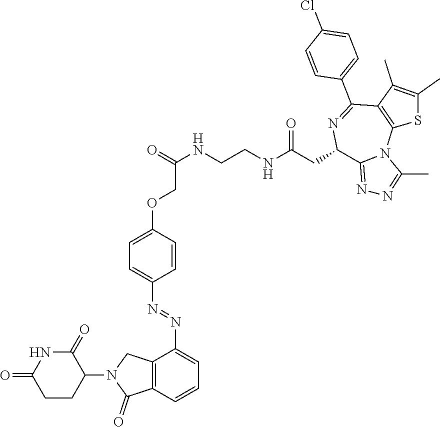 [SMILES: Cc1sc2c(c1C)C(c1ccc(Cl)cc1)=N[C@@H](CC(=O)NCCNC(=O)COc1ccc(/N=N/c3cccc4c3CN(C3CCC(=O)NC3=O)C4=O)cc1)c1nnc(C)n1-2]

Into a round bottom flask with dry (+)-JQ1 free acid (6.0 mg, 0.015 mmol, 1 eq.) were added S18 (17.3 mg, 0.030 mmol, 2 eq.) and HATU (8.7 mg, 0.027 mmol, 1.8 eq.) under nitrogen atmosphere. The solids were dissolved in dry DMF (1 mL). After addition of i-Pr₂NEt (17.6 mg, 0.137 mmol, 7.2 eq., 0.024 mL) the reaction was stirred for 15 h at room temperature. The mixture was then diluted with EtOAc (20 mL), separated against 5% LiCl (30 mL), extracted with EtOAc (2×20 mL), washed twice with 10% LiCl (2×20 mL) and brine (2×20 mL). The combined organic phases were was dried over Na₂SO₄ and concentrated under reduced pressure. Purification of the resulting crude product by flash column chromatography (CH₂Cl₂/MeOH gradient, 0→20% MeOH) gave PHOTAC-I-6 (12.4 mg, 0.015 mmol, 98%) as a yellow solid. R_{f}=0.10 [CH₂Cl₂:MeOH, 19:1]. ¹H NMR (400 MHz, Chloroform-d) δ=8.71 (s, 1H), 8.09 (d, J=7.8 Hz, 1H), 7.92 (d, J=7.5 Hz, 1H), 7.78 (d, J=8.4 Hz, 2H), 7.65 (t, J=7.7 Hz, 1H), 7.55 (s, 1H), 7.37 (d, J=8.2 Hz, 3H), 7.29 (d, J=8.3 Hz, 2H), 6.93 (d, J=8.6 Hz, 2H), 5.19 (dt, J=13.5, 4.6 Hz, 1H), 4.74 (d, J=18.9 Hz, 1H), 4.64 (dt, J=11.4, 5.9 Hz, 2H), 4.43 (s, 2H), 3.48 (dddd, J=51.8, 22.1, 11.6, 5.5 Hz, 6H), 2.93-2.74 (m, 2H), 2.61 (s, 3H), 2.45 (s, 1H), 2.35 (s, 3H), 2.24-2.14 (m, 1H), 1.62 (s, 3H) ppm. ¹³C NMR (100 MHz, CDCl₃) δ=171.67, 171.50, 169.95, 168.67, 168.28, 164.30, 160.24, 155.76, 150.14, 147.54, 147.01, 137.04, 136.62, 134.10, 133.41, 132.22, 131.10, 130.50, 130.00, 129.73, 129.69, 129.47, 128.88, 125.67, 124.97, 115.18, 67.39, 54.46, 52.25, 48.67, 39.80, 39.33, 39.11, 31.65, 23.44, 14.49, 13.22, 11.91 ppm. HRMS (ESI): calcd. for C₄₂H₃₉CIN₁₀NaO₆S⁺: 869.2355 m/z [M+Na]⁺; found: 869.2362 m/z [M+Na]⁺. LCMS (ESI): tᵣₑₜ=3.44 min (Z). 847 m/z [M+H]⁺. tᵣₑₜ=3.74 min (E). 847 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(4-(2-(4-((E)-(2-(2,6-dioxopiperidin-3-yl)-1-oxoisoindolin-4-yl)diazenyl)phenoxy)acetamido)butyl)acetamide (PHOTAC-I-7)

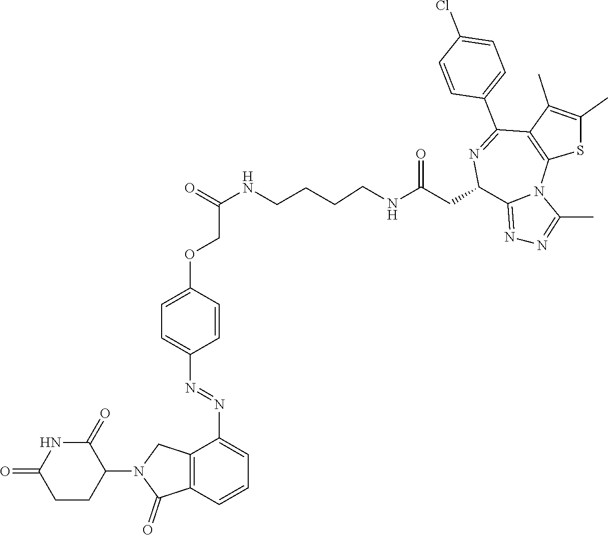 [SMILES: Cc1sc2c(c1C)C(c1ccc(Cl)cc1)=N[C@@H](CC(=O)NCCCCNC(=O)COc1ccc(/N=N/c3cccc4c3CN(C3CCC(=O)NC3=O)C4=O)cc1)c1nnc(C)n1-2]

Into a round bottom flask with dry (+)-JQ1 free acid (6.0 mg, 0.015 mmol, 1 eq.) were added S19 (18.2 mg, 0.030 mmol, 2 eq.) and HATU (8.7 mg, 0.027 mmol, 1.8 eq.) under nitrogen atmosphere. The solids were dissolved in dry DMF (1 mL). After addition of i-Pr₂NEt (13.9 mg, 0.108 mmol, 7.2 eq., 0.020 mL) the reaction was stirred for 16 h at room temperature. The mixture was then diluted with EtOAc (20 mL), separated against 5% LiCl (30 mL), extracted with EtOAc (2×20 mL), washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-7 (12.7 mg, 0.015 mmol, 97%) as a yellow solid. R_{f}=0.09 [CH₂Cl₂:MeOH, 19:1]. ¹H NMR (400 MHz, Chloroform-d) δ=8.57 (s, 1H), 8.15 (d, J=7.8 Hz, 1H), 7.95 (d, J=7.5 Hz, 1H), 7.87 (d, J=8.4 Hz, 2H), 7.67 (t, J=7.6 Hz, 1H), 7.39 (d, J=8.2 Hz, 2H), 7.32 (d, J=8.1 Hz, 2H), 7.01 (d, J=8.5 Hz, 2H), 6.92 (t, J=7.1 Hz, 1H), 6.82 (s, 1H), 5.23 (dt, J=13.4, 4.3 Hz, 1H), 4.80 (dd, J=18.1, 3.2 Hz, 1H), 4.71 (dd, J=18.1, 4.5 Hz, 1H), 4.62 (t, J=7.1 Hz, 1H), 4.51 (s, 2H), 3.56 (dd, J=14.3, 8.2 Hz, 1H), 3.34 (qd, J=17.8, 15.6, 6.3 Hz, 5H), 2.94-2.75 (m, 2H), 2.64 (s, 3H), 2.51-2.43 (m, 1H), 2.37 (s, 3H), 2.27-2.18 (m, 1H), 1.65 (s, 3H), 1.57 (s, 4H) ppm. ¹³C NMR (100 MHz, CDCl₃) δ=171.33, 170.67, 169.81, 168.65, 167.67, 164.26, 160.15, 155.72, 150.14, 147.78, 147.05, 137.03, 136.63, 134.15, 133.40, 132.22, 131.11, 130.58, 129.99, 129.75, 129.69, 129.52, 128.88, 125.86, 125.09, 115.25, 67.60, 54.63, 52.09, 48.45, 39.52, 39.12, 38.85, 31.70, 26.89, 26.79, 23.53, 14.51, 13.22, 11.93 ppm. HRMS (ESI): calcd. for C₄₄H₄₃CIKN₁₀O₆S⁺: 913.2408 m/z [M+K]⁺; found: 913.2419 m/z [M+K]⁺. LCMS (ESI): tᵣₑₜ=3.48 min (Z). 875 m/z [M+H]⁺. tᵣₑₜ=3.71 min (E). 875 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(6-(2-(4-((E)-(2-(2,6-dioxopiperidin-3-yl)-1-oxoisoindolin-4-yl)diazenyl)phenoxy)acetamido)hexyl)acetamide (PHOTAC-I-8)

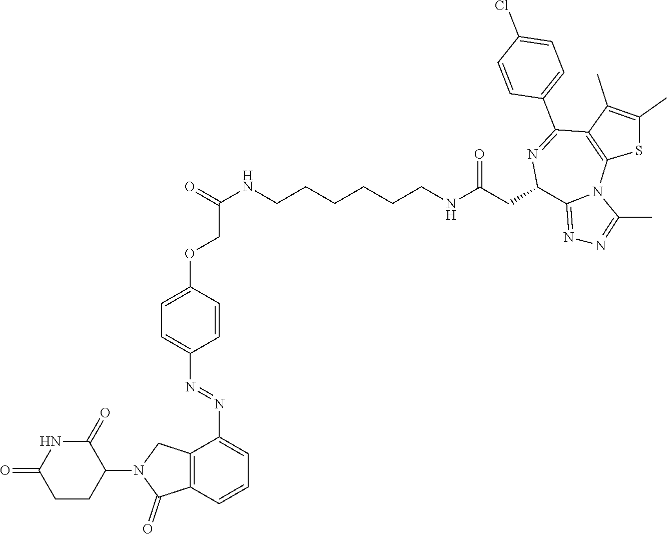 [SMILES: Cc1sc2c(c1C)C(c1ccc(Cl)cc1)=N[C@@H](CC(=O)NCCCCCCNC(=O)COc1ccc(/N=N/c3cccc4c3CN(C3CCC(=O)NC3=O)C4=O)cc1)c1nnc(C)n1-2]

Into a round bottom flask with dry (+)-JQ1 free acid (6.0 mg, 0.015 mmol, 1 eq.) were added S20 (19.0 mg, 0.030 mmol, 2 eq.) and HATU (8.7 mg, 0.027 mmol, 1.8 eq.) under nitrogen atmosphere. The solids were dissolved in dry DMF (1 mL). After addition of i-Pr₂NEt (13.9 mg, 0.108 mmol, 7.2 eq., 0.020 mL) the reaction was stirred for 18 h at room temperature. The mixture was then diluted with EtOAc (20 mL), separated against 5% LiCl (30 mL), extracted with EtOAc (2×20 mL), washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-I-8 (12.8 mg, 0.014 mmol, 95%) as a yellow solid. R_{f}=0.16 [CH₂Cl₂:MeOH, 19:1]. ¹H NMR (400 MHz, Chloroform-d) δ=8.51 (d, J=28.6 Hz, 1H), 8.17 (d, J=7.8 Hz, 1H), 7.97 (d, J=7.4 Hz, 1H), 7.91 (d, J=8.0 Hz, 2H), 7.68 (t, J=7.6 Hz, 1H), 7.39 (d, J=7.9 Hz, 2H), 7.31 (d, J=8.1 Hz, 2H), 7.03 (d, J=8.2 Hz, 2H), 6.76-6.66 (m, 1H), 6.61 (d, J=4.7 Hz, 1H), 5.24 (d, J=13.1 Hz, 1H), 4.84 (d, J=18.0 Hz, 1H), 4.73 (d, J=18.1 Hz, 1H), 4.59 (d, J=11.4 Hz, 3H), 3.55 (dd, J=14.0, 8.0 Hz, 1H), 3.29 (dd, J=28.3, 5.6 Hz, 5H), 2.96-2.77 (m, 2H), 2.64 (s, 3H), 2.46 (dt, J=13.0, 6.7 Hz, 1H), 2.38 (s, 3H), 2.30-2.18 (m, 1H), 1.65 (s, 3H), 1.51 (s, 4H), 1.31 (s, 4H) ppm. ¹³C NMR (100 MHz, CDCl₃) δ=171.27, 170.59, 169.77, 168.65, 167.62, 164.16, 160.14, 155.76, 150.07, 147.88, 147.09, 136.98, 136.69, 134.19, 133.41, 132.26, 131.05, 130.57, 129.97, 129.73, 129.67, 129.53, 128.86, 125.94, 125.11, 115.25, 67.69, 54.66, 52.07, 48.36, 39.58, 39.43, 38.92, 31.74, 29.47, 29.45, 26.34, 26.27, 23.58, 14.51, 13.23, 11.93 ppm. HRMS (ESI): calcd. for C₄₆H₄₇CIN₁₀NaO₆S⁺: 925.2981 m/z [M+Na]⁺; found: 925.2975 m/z [M+Na]⁺. LCMS (ESI): tᵣₑₜ=3.57 min (Z). 903 m/z [M+H]⁺. tᵣₑₜ=3.85 min (E). 903 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(4-(2-(2,6-dimethoxy-4-((E)-(2-(1-methyl-2,6-dioxopiperidin-3-yl)-1-oxoisoindolin-4-yl)diazenyl)phenoxy)acetamido)butyl)acetamide (Me-PHOTAC-I-3)

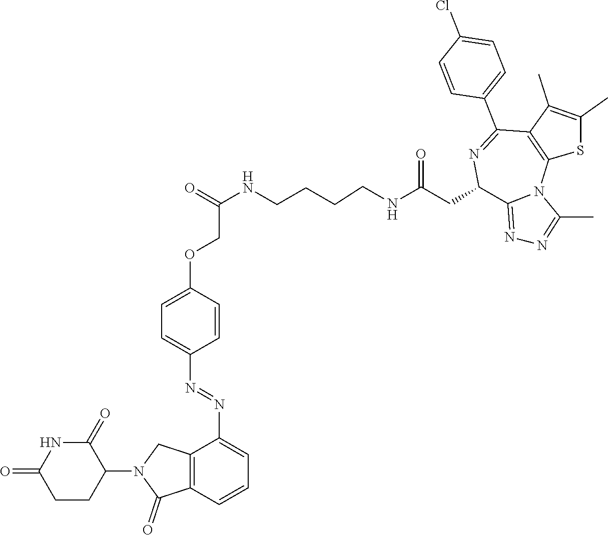 [SMILES: Cc1sc2c(c1C)C(c1ccc(Cl)cc1)=N[C@@H](CC(=O)NCCCCNC(=O)COc1ccc(/N=N/c3cccc4c3CN(C3CCC(=O)NC3=O)C4=O)cc1)c1nnc(C)n1-2]

Into a round bottom flask with dry PHOTAC-I-3 (10.0 mg, 0.011 mmol, 1 eq.) was added K2CO₃ (3.0 mg, 0.021 mmol, 2 eq.) under nitrogen atmosphere. The solids were dissolved in dry DMF (1 mL) and methyl iodide (1.8 mg, 0.013 mmol, 1.2 eq.) was added. The reaction was stirred for 16 h at room temperature. The mixture was then diluted with EtOAc (20 mL) and separated against 5% LiCl (20 mL). The aqueous phase was extracted with EtOAc (2×10 mL), and the combined organic phases were washed with 10% LiCl (2×20 mL) and brine (2×20 mL). The organic phase was dried over Na₂SO₄ and concentrated under reduced pressure. Purification of the resulting crude product by flash column chromatography (CH₂Cl₂/MeOH gradient, 0→20% MeOH) gave Me-PHOTAC-I-3 (5.2 mg, 0.005 mmol, 51%) as a yellow solid. R_{f}=0.37 [CH₂Cl₂:MeOH, 19:1]. ¹H NMR (400 MHz, DMSO-d₆) δ=δ 8.26-8.18 (m, 2H), 7.98-7.91 (m, 2H), 7.80 (t, J=7.7 Hz, 1H), 7.48 (d, J=8.4 Hz, 2H), 7.41 (d, J=8.5 Hz, 2H), 7.35 (s, 2H), 5.23 (dd, J=13.5, 5.0 Hz, 1H), 4.83 (d, J=19.1 Hz, 1H), 4.69 (d, J=19.0 Hz, 1H), 4.51 (ddd, J=7.9, 6.1, 1.3 Hz, 1H), 4.44 (s, 2H), 3.94 (s, 6H), 3.29-3.10 (m, 6H), 3.02 (s, 3H), 3.08-2.98 (m, 1H), 2.79 (ddd, J=17.3, 4.5, 2.4 Hz, 1H), 2.59 (s, 3H), 2.63-2.55 (m, 1H), 2.40 (s, 3H), 2.12-2.04 (m, 1H), 1.61 (s, 3H), 1.58-1.44 (m, 4H) ppm. ¹³C NMR (100 MHz, DMSO-d₆) δ=171.92, 170.64, 169.33, 167.98, 167.19, 163.05, 155.06, 152.58, 149.85, 148.28, 146.38, 139.31, 136.68, 135.23, 134.66, 133.81, 132.22, 130.74, 130.10, 129.83, 129.65, 129.56, 128.45, 128.41, 125.52, 100.61, 71.92, 56.37, 53.84, 52.36, 48.24, 38.20, 37.96, 37.60, 31.42, 26.69, 26.63, 26.62, 21.59, 14.04, 12.67, 11.28 ppm. HRMS (ESI): calcd. for C₄₇H₅₀CIN₁₀O₈S⁺: 949.3217 m/z [M+H]⁺; found: 949.3235 m/z [M+H]⁺. LCMS (ESI): tᵣₑₜ=3.94 min (Z). 949 m/z [M+H]⁺. tᵣₑₜ=4.19 min (E). 949 m/z [M+H]⁺.

### (1R)-3-(3,4-dimethoxyphenyl)-1-(3-(2-((3-(2-(4-((2-(2,6-dioxopiperidin-3-yl)-1-oxoisoindolin-4-yl)diazenyl)-2,6-dimethoxyphenoxy)acetamido)propyl)-amino)-2-oxoethoxy)phenyl)propyl-(2S)-1-(3,3-dimethyl-2-oxopentanoyl)-piperidine-2-carboxylate (PHOTAC-II-1)

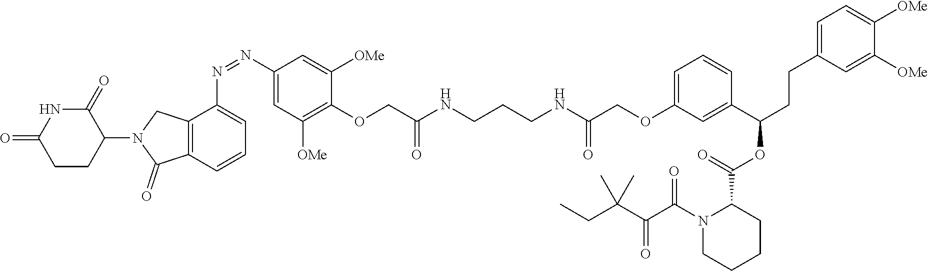 [SMILES: CCC(C)(C)C(=O)C(=O)N1CCCC[C@H]1C(=O)O[C@H](CCc1ccc(OC)c(OC)c1)c1cccc(OCC(=O)NCCCNC(=O)COc2c(OC)cc(/N=N\c3cccc4c3CN(C3CCC(=O)NC3=O)C4=O)cc2OC)c1]

Into a round bottom flask with dry 2-(3-((R)-3-(3,4-dimethoxyphenyl)-1-(((S)-1-(3,3-dimethyl-2-oxopentanoyl)piperidine-2-carbonyl)oxy)propyl)phenoxy)acetic acid (10.2 mg, 0.017 mmol, 1 eq.) were added S9 (22.8 mg, 0.035 mmol, 2 eq.) and HATU (12 mg, 0.031 mmol, 1.8 eq.) under nitrogen. The reaction was dissolved in dry DMF (1 mL). After addition of i-Pr₂NEt (17 mg, 0.13 mmol, 7.5 eq., 0.023 mL) the reaction was stirred for 14 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-II-1 (17.8 mg, 0.016 mmol, 92%) as a yellow solid. R_{f}=0.59 [CH₂Cl₂:MeOH, 9:1]. ¹H NMR (600 MHz, Chloroform-d) δ=8.33 (s, 1H), 8.20 (d, J=7.7 Hz, 1H), 8.00 (d, J=7.9 Hz, 1H), 7.84 (t, J=6.3 Hz, 1H), 7.71 (t, J=7.7 Hz, 1H), 7.37 (t, J=6.3 Hz, 1H), 7.28 (t, J=7.9 Hz, 1H), 7.21 (s, 2H), 7.00-6.92 (m, 2H), 6.87 (dd, J=8.2, 2.8 Hz, 1H), 6.80-6.73 (m, 1H), 6.69-6.65 (m, 2H), 5.77 (dd, J=8.0, 5.6 Hz, 1H), 5.30 (d, J=4.9 Hz, 1H), 5.25 (dd, J=13.4, 5.1 Hz, 1H), 4.85 (d, J=17.8 Hz, 1H), 4.72 (d, J=17.8 Hz, 1H), 4.60 (d, 2H), 4.46 (s, 2H), 3.96 (s, 6H), 3.86-3.81 (m, 6H), 3.47-3.33 (m, 5H), 3.16 (td, J=13.2, 3.2 Hz, 1H), 2.95-2.81 (m, 2H), 2.64-2.42 (m, 3H), 2.36 (d, J=13.9 Hz, 1H), 2.28-2.18 (m, 2H), 2.04 (dtd, J=11.6, 9.7, 4.8 Hz, 1H), 1.83-1.57 (m, 7H), 1.47 (qt, J=13.0, 4.0 Hz, 1H), 1.35 (tt, J=13.6, 3.6 Hz, 1H), 1.20 (d, J=11.9 Hz, 6H), 0.87 (t, J=7.4 Hz, 3H) ppm. ¹³C NMR (150 MHz, CDCl₃) δ=208.01, 171.22, 170.36, 169.77, 169.65, 168.55, 168.42, 167.38, 157.54, 152.75, 149.04, 148.99, 147.46, 146.88, 141.90, 139.76, 134.09, 133.49, 133.44, 130.08, 130.07, 129.61, 126.37, 120.25, 120.17, 114.23, 113.49, 111.81, 111.41, 100.56, 76.64, 72.73, 67.36, 56.50, 56.04, 55.96, 52.12, 51.39, 48.33, 46.82, 44.28, 38.28, 36.21, 36.08, 32.59, 31.73, 31.35, 29.84, 26.53, 25.06, 23.59, 23.57, 23.25, 21.32, 8.88 ppm. HRMS (ESI): calcd. for C₅₈H₇₃N₈O₁₅⁺: 1121.5189 m/z [M+NH₄]⁺; found: 1121.5246 m/z [M+NH₄]⁺. LCMS (ESI): tᵣₑₜ=4.56 min. 1104 m/z [M+H]⁺.

### (1R)-3-(3,4-dimethoxyphenyl)-1-(3-(2-((4-(2-(4-((2-(2,6-dioxopiperidin-3-yl)-1-oxoisoindolin-4-yl)diazenyl)-2,6-dimethoxyphenoxy)acetamido)butyl)-amino)-2-oxoethoxy)phenyl)propyl(2S)-1-(3,3-dimethyl-2-oxopentanoyl)-piperidine-2-carboxylate (PHOTAC-II-2)

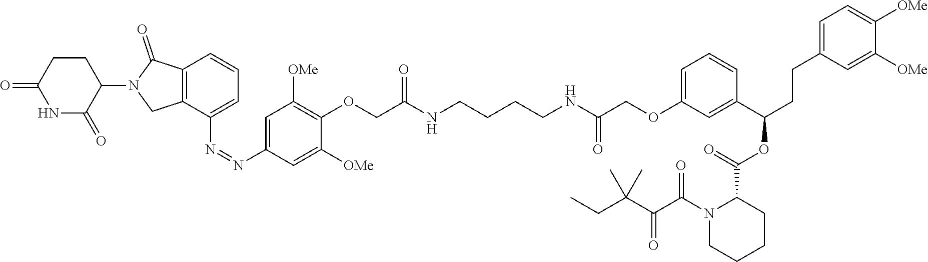 [SMILES: CCC(C)(C)C(=O)C(=O)N1CCCC[C@H]1C(=O)O[C@H](CCc1ccc(OC)c(OC)c1)c1cccc(OCC(=O)NCCCCNC(=O)COc2c(OC)cc(/N=N\c3cccc4c3CN(C3CCC(=O)NC3=O)C4=O)cc2OC)c1]

Into a round bottom flask with dry 2-(3-((R)-3-(3,4-dimethoxyphenyl)-1-(((S)-1-(3,3-dimethyl-2-oxopentanoyl)piperidine-2-carbonyl)oxy)propyl)phenoxy)acetic acid (10.2 mg, 0.018 mmol, 1 eq.) were added 3 (23.3 mg, 0.035 mmol, 2 eq.) and HATU (12 mg, 0.031 mmol, 1.8 eq.) under nitrogen. The reaction was dissolved in dry DMF (1 mL). After addition of i-Pr₂NEt (17 mg, 0.13 mmol, 7.5 eq., 0.023 mL) the reaction was stirred for 14 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-II-2 (17.7 mg, 0.016 mmol, 91%) as a yellow solid. R_{f}=0.59 [CH₂Cl₂:MeOH, 9:1]. ¹H NMR (600 MHz, Chloroform-d) δ=8.35 (s, 1H), 8.19 (dd, J=7.8, 1.0 Hz, 1H), 7.99 (dd, J=7.5, 1.0 Hz, 1H), 7.74-7.67 (m, 2H), 7.28 (t, J=7.9 Hz, 1H), 7.22 (s, 2H), 6.99-6.93 (m, 2H), 6.84-6.75 (m, 3H), 6.70-6.64 (m, 2H), 5.79-5.73 (m, 1H), 5.33-5.27 (m, 1H), 5.24 (dd, J=13.4, 5.1 Hz, 1H), 4.85 (d, J=17.9 Hz, 1H), 4.72 (d, J=17.8 Hz, 1H), 4.60 (s, 2H), 4.48 (s, 2H), 3.98 (s, 6H), 3.86-3.82 (m, 6H), 3.44-3.32 (m, 5H), 3.16 (td, J=13.2, 3.2 Hz, 1H), 2.92-2.80 (m, 2H), 2.65-2.43 (m, 3H), 2.36 (d, J=14.0 Hz, 1H), 2.28-2.18 (m, 2H), 2.04 (ddt, J=13.9, 10.1, 5.9 Hz, 1H), 1.78-1.59 (m, 9H), 1.48 (qt, J=13.2, 4.4 Hz, 1H), 1.35 (tt, J=13.3, 3.4 Hz, 1H), 1.20 (d, J=8.2 Hz, 6H), 0.87 (t, J=7.5 Hz, 3H) ppm. ¹³C NMR (150 MHz, CDCl₃) δ=208.03, 171.25, 169.78, 169.69, 169.68, 168.55, 168.20, 167.37, 157.43, 152.73, 149.00, 148.99, 147.47, 146.87, 142.04, 139.81, 134.13, 133.46, 133.44, 130.13, 130.01, 129.59, 126.33, 120.26, 120.24, 113.88, 113.63, 111.82, 111.41, 100.58, 76.54, 72.82, 67.37, 56.50, 56.04, 55.96, 52.15, 51.37, 48.38, 46.82, 44.27, 38.82, 38.69, 38.29, 32.59, 31.72, 31.34, 27.22, 27.20, 26.52, 25.06, 23.56, 23.53, 23.31, 21.29, 8.89 ppm. HRMS (ESI): calcd. for C₅₉H₇₂N₇O₁₅⁺: 1118.5081 m/z [M+H]⁺; found: 1118.5081 m/z [M+H]⁺. LCMS (ESI): tᵣₑₜ=4.56 min. 1118 m/z [M+H]⁺.

### (1R)-3-(3,4-dimethoxyphenyl)-1-(3-(2-((5-(2-(4-((Z)-(2-(2,6-dioxopiperidin-3-yl)-1-oxoisoindolin-4-yl)diazenyl)-2,6-dimethoxyphenoxy)acetamido)pentyl)amino)-2-oxoethoxy)phenyl)propyl (25)-1-(3,3-dimethyl-2-oxopentanoyl)piperidine-2-carboxylate (PHOTAC-II-3)

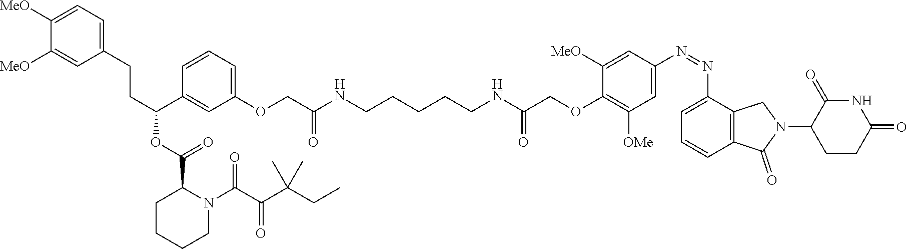 [SMILES: CCC(C)(C)C(=O)C(=O)N1CCCC[C@H]1C(=O)O[C@H](CCc1ccc(OC)c(OC)c1)c1cccc(OCC(=O)NCCCCCNC(=O)COc2c(OC)cc(/N=N\c3cccc4c3CN(C3CCC(=O)NC3=O)C4=O)cc2OC)c1]

Into a round bottom flask with dry 2-(3-((R)-3-(3,4-dimethoxyphenyl)-1-(((S)-1-(3,3-dimethyl-2-oxopentanoyl)piperidine-2-carbonyl)oxy)propyl)phenoxy)acetic acid (10.0 mg, 0.018 mmol, 1 eq.) were added S10 (25.1 mg, 0.034 mmol, 2 eq.) and HATU (11.7 mg, 0.031 mmol, 1.8 eq.) under nitrogen. The reaction was dissolved in dry DMF (1 mL). After addition of i-Pr₂NEt (15.5 mg, 0.12 mmol, 7 eq., 0.021 mL) the reaction was stirred for 14 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-II-3 (14.4 mg, 0.013 mmol, 74%) as a yellow solid. R_{f}=0.28 [CH₂Cl₂:MeOH, 19:1]. ¹H NMR (600 MHz, Chloroform-d) δ=8.36 (d, J=3.6 Hz, 1H), 8.20 (dd, J=7.8, 1.0 Hz, 1H), 8.00 (dd, J=7.5, 1.0 Hz, 1H), 7.71 (t, J=7.5 Hz, 2H), 7.29 (t, J=7.9 Hz, 1H), 7.22 (s, 2H), 7.00-6.92 (m, 2H), 6.82 (dd, J=8.2, 2.6 Hz, 1H), 6.80-6.72 (m, 2H), 6.71-6.63 (m, 2H), 5.77 (dd, J=8.2, 5.5 Hz, 1H), 5.31 (d, J=6.1 Hz, 1H), 5.25 (dd, J=13.4, 5.1 Hz, 1H), 4.86 (d, J=17.8 Hz, 1H), 4.72 (d, J=17.8 Hz, 1H), 4.61 (s, 2H), 4.47 (s, 2H), 3.98 (s, 6H), 3.87-3.81 (m, 6H), 3.35 (q, J=6.7 Hz, 5H), 3.21-3.12 (m, 1H), 3.04-2.90 (m, 1H), 2.85 (ddd, J=18.1, 13.3, 5.2 Hz, 1H), 2.59-2.41 (m, 3H), 2.36 (d, J=14.0 Hz, 1H), 2.24 (m, 2H), 2.04 (m, 1H), 1.81-1.57 (m, 9H), 1.48 (m, 1H), 1.44-1.30 (m, 3H), 1.20 (d, J=8.0 Hz, 6H), 0.87 (t, J=7.4 Hz, 3H) ppm. ¹³C NMR (150 MHz, CDCl₃) δ=208.03, 171.19, 169.79, 169.68, 169.64, 168.55, 168.17, 167.38, 157.45, 152.72, 149.03, 149.00, 147.49, 146.89, 142.04, 139.85, 134.11, 133.45, 133.42, 130.14, 130.06, 129.60, 126.36, 120.27, 120.24, 113.85, 113.70, 111.83, 111.42, 100.59, 76.54, 72.81, 67.38, 56.51, 56.05, 55.97, 52.13, 51.37, 48.33, 46.83, 44.28, 39.04, 38.91, 38.30, 32.60, 31.75, 31.35, 29.44, 29.35, 26.53, 25.07, 24.24, 23.59, 23.57, 23.32, 21.30, 8.90 ppm. HRMS (APCI): calcd. for C₆₀H₇₄N₇O₁₅⁺: 1132.5273 m/z [M+H]⁺; found: 1132.5217 m/z [M+H]⁺. LCMS (ESI): tᵣₑₜ=4.67 (Z) min. 566.7 m/z [M+2H]²⁺. tᵣₑₜ=4.83 (E) min. 566.7 m/z [M+2H]²⁺.

### (1R)-3-(3,4-dimethoxyphenyl)-1-(3-(2-((6-(2-(4-((Z)-(2-(2,6-dioxopiperidin-3-yl)-1-oxoisoindolin-4-yl)diazenyl)-2,6-dimethoxyphenoxy)acetamido)hexyl)amino)-2-oxoethoxy)phenyl)propyl (25)-1-(3,3-dimethyl-2-oxopentanoyl)piperidine-2-carboxylate (PHOTAC-II-4)

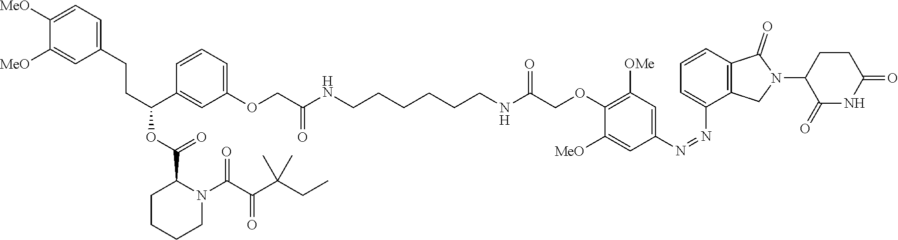 [SMILES: CCC(C)(C)C(=O)C(=O)N1CCCC[C@H]1C(=O)O[C@H](CCc1ccc(OC)c(OC)c1)c1cccc(OCC(=O)NCCCCCCNC(=O)COc2c(OC)cc(/N=N\c3cccc4c3CN(C3CCC(=O)NC3=O)C4=O)cc2OC)c1]

Into a round bottom flask with dry 2-(3-((R)-3-(3,4-dimethoxyphenyl)-1-(((S)-1-(3,3-dimethyl-2-oxopentanoyl)piperidine-2-carbonyl)oxy)propyl)phenoxy)acetic acid (10.0 mg, 0.018 mmol, 1 eq.) were added S11 (23.8 mg, 0.034 mmol, 2 eq.) and HATU (11.7 mg, 0.031 mmol, 1.8 eq.) under nitrogen. The reaction was dissolved in dry DMF (1 mL). After addition of i-Pr₂NEt (15.5 mg, 0.12 mmol, 7 eq., 0.021 mL) the reaction was stirred for 14 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-II-4 (16.9 mg, 0.015 mmol, 86%) as a yellow solid. R_{f}=0.33 [CH₂Cl₂:MeOH, 19:1]. ¹H NMR (600 MHz, Chloroform-d) δ=8.30 (s, 1H), 8.20 (dd, J=7.9, 1.0 Hz, 1H), 8.00 (dd, J=7.5, 1.0 Hz, 1H), 7.71 (t, J=7.7 Hz, 1H), 7.65 (t, J=5.9 Hz, 1H), 7.29 (td, J=7.9, 3.6 Hz, 1H), 7.22 (s, 2H), 7.00-6.88 (m, 2H), 6.85-6.75 (m, 2H), 6.72 (t, J=6.0 Hz, 1H), 6.70-6.64 (m, 2H), 5.82-5.69 (m, 1H), 5.31 (d, J=5.9 Hz, 1H), 5.26 (dd, J=13.4, 5.1 Hz, 1H), 4.85 (d, J=17.8 Hz, 1H), 4.72 (d, J=17.8 Hz, 1H), 4.60 (s, 2H), 4.48 (d, J=2.5 Hz, 2H), 3.98 (s, 6H), 3.85 (dd, J=4.8, 2.5 Hz, 6H), 3.40-3.30 (m, 5H), 3.16 (td, J=13.2, 3.1 Hz, 1H), 3.01-2.78 (m, 2H), 2.61-2.42 (m, 3H), 2.36 (d, J=14.0 Hz, 1H), 2.24 (dddd, J=17.8, 15.2, 7.8, 5.4 Hz, 2H), 2.10-1.99 (m, 1H), 1.80-1.52 (m, 9H), 1.48 (dt, J=13.1, 4.0 Hz, 1H), 1.38 (tt, J=10.4, 4.7 Hz, 5H), 1.21 (d, J=8.3 Hz, 6H), 0.87 (t, J=7.4 Hz, 3H) ppm. ¹³C NMR (150 MHz, CDCl₃) δ=208.01, 171.17, 169.79, 169.63, 169.50, 168.54, 168.08, 167.37, 157.49, 152.74, 149.01, 148.99, 147.50, 146.91, 142.03, 139.91, 134.10, 133.45, 133.45, 130.14, 130.04, 129.61, 126.36, 120.27, 120.23, 113.89, 113.70, 111.84, 111.43, 100.59, 76.54, 72.88, 67.44, 56.50, 56.06, 55.97, 52.12, 51.37, 48.29, 46.83, 44.28, 39.12, 38.98, 38.30, 32.61, 31.74, 31.35, 29.74, 29.64, 26.69, 26.67, 26.52, 25.08, 23.61, 23.58, 23.32, 21.30, 8.90 ppm. HRMS (APCI): calcd. for C₆₁H₇₆N₇O₁₅⁺: 1146.5394 m/z [M+H]⁺; found: 1146.5392 m/z [M+H]⁺. LCMS (ESI): tᵣₑₜ=4.89 min. 573 m/z [M+2E1]²⁺.

### (1R)-3-(3,4-dimethoxyphenyl)-1-(3-(2-((2-(2-(4-((Z)-(2-(2,6-dioxopiperidin-3-yl)-1-oxoisoindolin-4-yl)diazenyl)phenoxy)acetamido)ethyl)amino)-2-oxoethoxy)phenyl)propyl (25)-1-(3,3-dimethyl-2-oxopentanoyl)piperidine-2-carboxylate (PHOTAC-II-5)

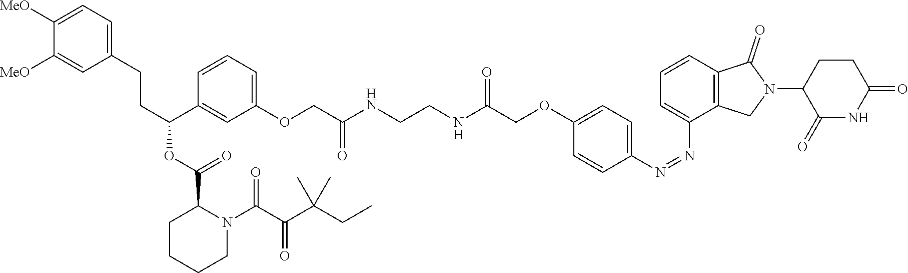 [SMILES: CCC(C)(C)C(=O)C(=O)N1CCCC[C@H]1C(=O)O[C@H](CCc1ccc(OC)c(OC)c1)c1cccc(OCC(=O)NCCNC(=O)COc2ccc(/N=N\c3cccc4c3CN(C3CCC(=O)NC3=O)C4=O)cc2)c1]

Into a round bottom flask with dry 2-(3-((R)-3-(3,4-dimethoxyphenyl)-1-(((S)-1-(3,3-dimethyl-2-oxopentanoyl)piperidine-2-carbonyl)oxy)propyl)phenoxy)acetic acid (8.0 mg, 0.014 mmol, 1 eq.) were added S18 (15.9 mg, 0.027 mmol, 2 eq.) and HATU (9.4 mg, 0.025 mmol, 1.8 eq.) under nitrogen. The reaction was dissolved in dry DMF (1 mL). After addition of i-Pr₂NEt (12.4 mg, 0.096 mmol, 7 eq., 0.02 mL) the reaction was stirred for 15 h at room temperature. The mixture was then diluted with EtOAc (20 mL), separated against 5% LiCl (20 mL), extracted with EtOAc (2×20 mL) and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave PHOTAC-II-5 (9.1 mg, 0.009 mmol, 65%) as a yellow solid. R_{f}=0.17 [CH₂Cl₂:MeOH, 19:1]. ¹H NMR (600 MHz, DMSO-d₆) δ=11.02 (s, 1H), 8.30 (d, J=5.0 Hz, 1H), 8.21 (d, J=5.3 Hz, 1H), 8.17 (d, J=7.8 Hz, 1H), 7.97 (d, J=8.5 Hz, 2H), 7.89 (d, J=7.4 Hz, 1H), 7.78 (t, J=7.6 Hz, 1H), 7.29 (q, J=7.9 Hz, 1H), 7.19-7.15 (m, 2H), 6.96 (dd, J=4.3, 2.2 Hz, 2H), 6.92-6.88 (m, 1H), 6.82 (d, J=8.1 Hz, 1H), 6.76-6.73 (m, 1H), 6.65 (d, J=8.2 Hz, 1H), 5.71-5.65 (m, 1H), 5.16 (m, 2H), 4.78 (d, J=19.0 Hz, 1H), 4.67 (d, J=18.9 Hz, 1H), 4.60 (s, 2H), 4.47 (s, 2H), 3.71 (s, 3H), 3.69 (s, 3H), 3.29-3.19 (m, 5H), 3.10 (t, J=12.3 Hz, 1H), 2.94 (ddd, J=17.9, 13.4, 5.4 Hz, 1H), 2.61 (d, J=18.2 Hz, 1H), 2.57-2.45 (m, 4H), 2.23 (d, J=13.3 Hz, 1H), 2.20-2.08 (m, 1H), 2.06-1.98 (m, 2H), 1.74-1.50 (m, 4H), 1.40-1.29 (m, 1H), 1.14 (s, 3H), 1.12 (s, 3H), 1.04 (d, J=6.4 Hz, 1H), 0.78 (t, J=7.4 Hz, 3H) ppm. ¹³C NMR (150 MHz, DMSO) δ=207.64, 172.91, 171.00, 169.32, 167.92, 167.46, 167.22, 166.82, 160.86, 157.72, 148.63, 147.06, 146.68, 146.58, 141.63, 134.28, 133.74, 133.13, 129.72, 129.57, 128.49, 124.99, 124.74, 119.91, 119.00, 115.41, 114.13, 112.98, 112.10, 111.87, 75.99, 67.10, 66.97, 55.47, 55.32, 51.66, 50.89, 48.24, 46.17, 43.84, 38.23, 38.16, 37.59, 31.93, 31.25, 30.62, 26.06, 24.36, 22.88, 22.60, 22.33, 20.77, 8.58 ppm. HRMS (APCI): calcd. for C₅₅H₆₄N₇O₁₃⁺: 1030.4557 m/z [M+H]⁺; found: 1030.4565 m/z [M+H]⁺. LCMS (ESI): tᵣₑₜ=4.70 min. 1030 m/z [M+H]⁺.

### (E)-N-(4-((4-aminophenyl)diazenyl)phenyl)-2-((2-(2,6-dioxopiperidin-3-yl)-1,3-dioxoisoindolin-4-yl)oxy)acetamide (S21)

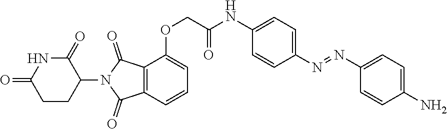 [SMILES: Nc1ccc(/N=N/c2ccc(NC(=O)COc3cccc4c3C(=O)N(C3CCC(=O)NC3=O)C4=O)cc2)cc1]

To a solution of thalidomide-4-hydroxyacetate (16.7 mg, 50 μmol, 1.0 eq.) and 4,4′-diaminoazobenzene (32 mg, 150 μmol, 3.0 eq.) in THF (1.9 mL) was added HOBt (6.8 mg, 50 μmol, 1.0 eq.), PyBOP (52 mg, 100 μmol, 2.0 eq.) and triethylamine (35 μL, 26 mg, 250 μmol, 5.0 eq.) at room temperature. The reaction was stirred overnight, upon which the reaction solution was diluted with EtOAc, washed with water, sodium bicarbonate, and brine. The organic layer was dried over sodium sulfate and concentrated in vacuo. The crude product was purified by column chromatography over SiO₂ using 0%→10% MeOH in CH₂Cl₂ as the eluent to afford the desired product S21 (21 mg, 40 μmol, 79%) as a highly insoluble brown solid. R_{f}=0.51 [CH₂Cl₂:MeOH, 19:1]. ¹H NMR (400 MHz, DMSO-d₆) δ=11.13 (s, 1H), 10.35 (s, 1H), 7.83 (t, J=7.9 Hz, 1H), 7.76 (s, 4H), 7.63 (d, J=8.4 Hz, 2H), 7.51 (t, J=7.5 Hz, 2H), 6.66 (d, J=8.5 Hz, 2H), 6.05 (s, 2H), 5.15 (dd, J=12.9, 5.4 Hz, 1H), 5.05 (s, 2H), 2.99-2.80 (m, 1H), 2.59 (t, 2H), 2.07 (d, J=13.0 Hz, 2H) ppm. ¹³C NMR (100 MHz, DMSO) δ=172.8, 169.9, 166.7, 165.9, 165.5, 155.2, 152.5, 148.5, 142.8, 139.4, 137.0, 133.1, 124.9, 122.6, 120.5, 119.6, 116.7, 116.1, 113.4, 67.6, 48.8, 31.0, 22.0 ppm. HRMS (ESI): calcd. for C₂₇H₂₂N₆NaO₆⁺: 549.1493 m/z [M+Na]⁺; found: 549.1478 m/z [M+Na]⁺. LCMS T_{R}=3.521 min.

### 2-((S)-4-(4-chlorophenyl)-2,3,9-trimethyl-6H-thieno[3,2-f][1,2,4]triazolo[4,3-a][1,4]diazepin-6-yl)-N-(4-((E)-(4-(2-((2-(2,6-dioxopiperidin-3-yl)-1,3-dioxoisoindolin-4-yl)oxy)acetamido)phenyl)diazenyl)phenyl)acetamide (PHOTAC-I-10)

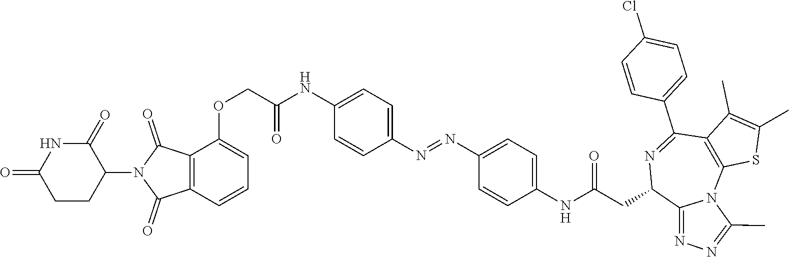 [SMILES: Cc1sc2c(c1C)C(c1ccc(Cl)cc1)=N[C@@H](CC(=O)Nc1ccc(/N=N/c3ccc(NC(=O)COc4cccc5c4C(=O)N(C4CCC(=O)NC4=O)C5=O)cc3)cc1)c1nnc(C)n1-2]

To a solution of S37 (5.0 mg, 9.5 μmol, 1.0 eq.) and (+)-JQ1 free acid (4.2 mg, 10.4 μmol, 1.1 eq.) DCE (1.0 mL) was added TBTU (4.0 mg, 12.3 μmol, 1.3 eq.) and DIPEA (2 μL, 14.2 μmol, 1.5 eq.) at room temperature. The reaction was allowed to stir at room temperature overnight. Upon completion, the reaction was diluted with EtOAc and washed with water, NaHCO₃ and brine. The organics were dried over sodium sulfate and concentrated in vacuo. The residue was purified by column chromatography over SiO₂ using 0%→10% MeOH in CH₂Cl₂ as the eluent to afford the desired product PHOTAC-I-10 (3.0 mg, 3.3 μmol, 35%) as an orange amorphous solid. R_{F}=0.32 [CH₂Cl₂:MeOH, 95:5]. ¹H NMR (400 MHz, Chloroform-d) δ=9.46 (s, 1H), 9.36 (d, J=6.0 Hz, 1H), 8.21 (d, J=10.7 Hz, 1H), 7.82 (d, J=1.9 Hz, 4H), 7.75 (dd, J=8.9, 2.3 Hz, 2H), 7.69 (ddd, J=8.5, 7.5, 2.0 Hz, 1H), 7.66-7.60 (m, 2H), 7.51 (dd, J=7.4, 3.5 Hz, 1H), 7.35 (d, J=8.4 Hz, 2H), 7.26 (d, J=8.3 Hz, 2H), 7.17 (d, J=1.3 Hz, 1H), 4.97 (ddd, J=12.3, 5.4, 2.0 Hz, 1H), 4.71 (s, 2H), 4.63 (dd, J=8.7, 5.4 Hz, 1H), 3.87-3.76 (m, 1H), 3.49 (dd, J=14.3, 5.3 Hz, 1H), 2.92-2.85 (m, 1H), 2.85-2.78 (m, 1H), 2.78-2.71 (m, 1H), 2.63 (s, 3H), 2.35 (s, 3H), 2.17-2.08 (m, 1H), 1.63 (s, 3H) ppm. ¹³C NMR (100 MHz, CDCl₃) δ=170.9, 170.9, 169.1, 168.1, 166.7, 166.5, 165.0, 164.4, 155.8, 154.4, 150.3, 149.6, 149.0, 140.9, 139.6, 137.4, 137.2, 136.5, 133.6, 132.2, 131.3, 131.1, 130.6, 130.1, 128.9, 124.0, 123.9, 120.1, 120.0, 119.9, 118.0, 68.5, 54.7, 49.6, 40.8, 31.6, 22.7, 14.6, 13.3, 12.0 ppm. HRMS (ESI): calcd. for C₄₆H₃₇ClN₁₀O₇SNa⁺: 931.2148 m/z [M+Na]⁺; found: 931.2167 m/z [M+Na]⁺. LCMS T_{R}=4.642 min.

### (1R)-3-(3,4-dimethoxyphenyl)-1-(3-(2-((4-((E)-(4-(2-((2-(2,6-dioxopiperidin-3-yl)-1,3-dioxoisoindolin-4-yl)oxy)acetamido)phenyl)diazenyl)phenyl)amino)-2-oxoethoxy)phenyl)propyl (2S)-1-(3,3-dimethyl-2-oxopentanoyl)piperidine-2-carboxylate (PHOTAC-II-6)

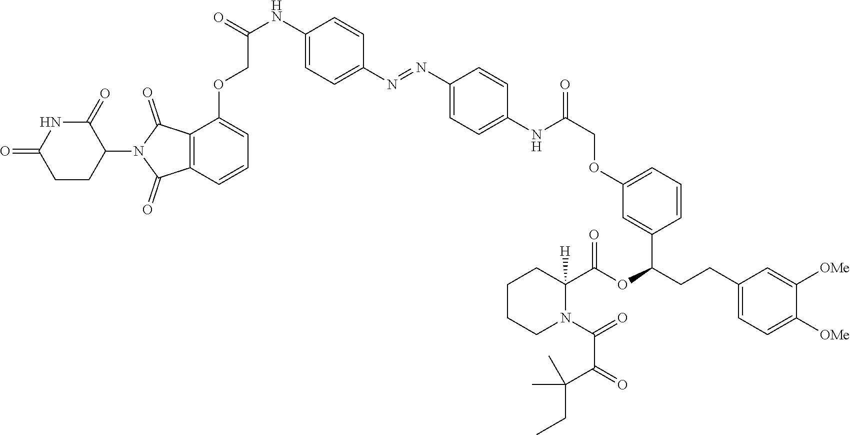 [SMILES: CCC(C)(C)C(=O)C(=O)N1CCCC[C@H]1C(=O)O[C@H](CCc1ccc(OC)c(OC)c1)c1cccc(OCC(=O)Nc2ccc(/N=N/c3ccc(NC(=O)COc4cccc5c4C(=O)N(C4CCC(=O)NC4=O)C5=O)cc3)cc2)c1]

To a solution of S37 (6.0 mg, 11.4 μmol, 1.0 eq.) and SLF free acid (7.3 mg, 12.5 μmol, 1.1 eq.) in DMF (1.2 mL) was added HATU (5.6 mg, 14.8 μmol, 1.3 eq.) and DIPEA (3 μL, 17.1 μmol, 1.5 eq.) at room temperature. The reaction was allowed to stir at room temperature overnight. Upon completion, the reaction was diluted with EtOAc and washed with water, NaHCO₃ and brine. The organics were dried over sodium sulfate and concentrated to afford an orange amorphous solid. The residue was purified by column chromatography using 0%→50% acetone in CH₂Cl₂ to afford the desired product PHOTAC-II-6 (8.1 mg, 7.4 μmol, 65%) as an orange amorphous solid. R_{f}=0.15 [CH₂Cl₂:Acetone, 9:1]. ¹H NMR (400 MHz, Chloroform-d) δ=9.55 (s, 1H), 8.55 (d, J=22.1 Hz, 1H), 8.24 (d, J=3.7 Hz, 1H), 7.98-7.90 (m, 6H), 7.82-7.72 (m, 3H), 7.58 (d, J=7.3 Hz, 1H), 7.34 (t, J=7.9 Hz, 1H), 7.24 (s, 1H), 7.11-7.03 (m, 1H), 7.03-6.99 (m, 1H), 6.93 (dd, J=8.3, 2.4 Hz, 1H), 6.82-6.73 (m, 1H), 6.67 (d, J=6.2 Hz, 2H), 5.81 (dd, J=8.0, 5.3 Hz, 1H), 5.34 (d, J=5.6 Hz, 1H), 5.04 (dd, J=12.2, 5.3 Hz, 1H), 4.79 (s, 2H), 4.65 (s, 2H), 3.85 (d, J=5.9 Hz, 6H), 3.35 (s, 2H), 3.22-3.11 (m, 1H), 3.01-2.74 (m, 4H), 2.67-2.51 (m, 2H), 2.39-2.20 (m, 2H), 2.17 (d, J=2.1 Hz, 4H), 2.07 (td, J=9.0, 7.2, 3.9 Hz, 1H), 1.84-1.56 (m, 5H), 1.52-1.28 (m, 0H), 1.25 (s, 7H), 1.21 (d, J=1.8 Hz, 5H), 0.87 (t, J=7.4 Hz, 4H) ppm. ¹³C NMR (100 MHz, CDCl₃) δ=208.1, 170.9, 169.9, 168.0, 167.4, 166.6, 166.5, 166.3, 165.0, 157.3, 154.4, 149.5, 149.5, 149.0, 147.5, 142.2, 139.9, 139.3, 137.4, 133.6, 133.4, 130.3, 124.1, 124.0, 120.7, 120.3, 120.3, 120.1, 120.1, 118.6, 118.0, 114.1, 113.8, 111.9, 111.9, 111.4, 76.5, 68.5, 67.7, 56.1, 56.0, 53.9, 53.6, 51.4, 49.6, 46.9, 44.3, 38.3, 32.6, 31.6, 31.3, 31.1, 29.4, 26.5, 25.1, 23.5, 23.4, 22.7, 21.2, 14.3, 8.9 ppm. HRMS (ESI): calcd. for C₅₉H₆₂N₇O₁₄⁺: 1092.4355 m/z [M+H]⁺; found: 1092.4334 m/z [M+H]⁺. LCMS T_{R}=5.265 min.

### tert-butyl (E)-(4-((4-nitrophenyl)diazenyl)benzyl)carbamate (S22)

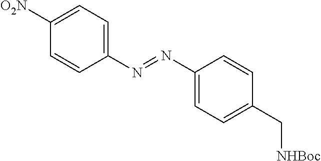 [SMILES: CC(C)(C)OC(=O)NCc1ccc(/N=N/c2ccc([N+](=O)[O-])cc2)cc1]

The following procedure was carried out in two steps:

Oxone Oxidation: To a solution of 4-nitroaniline (566 mg, 4.1 mmol, 1.0 eq) in CH₂Cl₂ (14.6 mL) was added a solution of oxone (2.5 g, 4.1 mmol, 1.0 eq) in water (14.6 mL). The biphasic mixture was stirred vigorously under N₂ atmosphere. After 3 hours, phases were separated and the aqueous phase was extracted with CH₂Cl₂. The organic phases were combined and then washed with 1 M HCl, sat. NaCl, dried over Na₂SO₄ and concentrated under reduced pressure to approximately 5 mL. The resulting yellow-black solution of nitrosobenzene in CH₂Cl₂ was carried on to the next step immediately.

Mills Reaction: To the nitrosobenzene solution in CH₂Cl₂, prepared as described above, was added sequentially tert-butyl (4-aminobenzyl)carbamate (910 mg, 4.1 mmol, 1.0 eq) and glacial AcOH (1.2 mL, 20 mmol, 5.000 eq). The reaction mixture was allowed to stir for 15 hours under N2 atmosphere, after which time the reaction mixture was found to be an orange-black suspension. EtOAc was added and the organic phase was washed with 1 M NaOH, sat. NaHCO₃, sat. NaCl. The organic phase was then dried over Na₂SO₄ and concentrated under reduced pressure. Crude material was purified by flash column chromatography over SiO₂ using a gradient from 1%→5%→10% EtOAc in Hexanes as the eluent, affording S22 (900 mg, 2.5 mmol, 62%) as a crystalline red solid. R_{f}=0.24 [Hexanes:EtOAc, 85:15]. ¹H NMR (400 MHz, Chloroform-d) δ=8.37 (d, J=8.8 Hz, 2H), 8.02 (d, J=8.9 Hz, 2H), 7.94 (d, J=8.2 Hz, 2H), 7.46 (d, J=8.2 Hz, 2H), 4.98 (s, 1H), 4.42 (d, J=5.3 Hz, 2H), 1.48 (s, 2H) ppm. ¹³C NMR (101 MHz, CDCl₃) δ=156.0, 155.8, 151.8, 148.8, 144.0, 128.2, 124.9, 123.9, 123.6, 80.0, 44.5, 28.5 ppm. HRMS (APCI): calcd. for C₁₃H₁₃N₄O₂⁺: 257.1033 m/z [M-Boc+H]⁺; found: 257.1041 m/z [M-Boc+H]⁺. LCMS T_{R}=4.736 min.

### tert-butyl (E)-(4-((4-aminophenyl)diazenyl)benzyl)carbamate (S23)

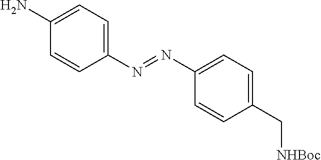 [SMILES: CC(C)(C)OC(=O)NCc1ccc(/N=N/c2ccc(N)cc2)cc1]

To a solution of S22 (100.0 mg, 270 μmol, 1.0 eq.) in dioxane (4.0 mL) and water (0.4 mL) in a pressure tube was added Na₂S.9H₂O (202 mg, 842 μmol, 3.0 eq.). The reaction was sealed and heated to 85° C. After 1 hour, the reaction was diluted with water and the aqueous layer was extracted 3 times with EtOAc. The organics were combined, washed with brine, dried over sodium sulfate and concentrated in vacuo to afford an orange-red solid. The reaction was loaded onto isolute and purified by column chromatography over SiO₂ using a stepped gradient from 9:1→41:1 Hexanes/EtOAc as the eluent to afford S23 (70.0 mg, 215 μmol, 76%) as a pale orange solid. R_{f}=0.14 [Hexanes:EtOAc, 8:2]. ¹H NMR (400 MHz, Chloroform-d) δ=7.80 (dd, J=8.6, 3.0 Hz, 4H), 7.37 (d, J=8.1 Hz, 2H), 6.72 (d, J=8.7 Hz, 2H), 4.96 (s, 1H), 4.36 (d, J=6.0 Hz, 2H), 4.21-3.92 (m, 2H), 1.47 (s, 10H). ¹³C NMR (101 MHz, CDCl₃) δ=156.0, 152.3, 149.8, 145.6, 140.8, 128.1, 125.2, 122.7, 114.7, 79.7, 44.5, 28.5. HRMS (APCI): calcd. for C₁₈H₂₃N₄O₄⁺: 327.1816 m/z [M+H]⁺; found: 327.1804 m/z [M+H]⁺. LCMS T_{R}=4.032 min.

### tert-butyl (E)-(4-((4-(2-((2-(2,6-dioxopiperidin-3-yl)-1,3-dioxoisoindolin-4-yl)oxy)acetamido)phenyl)diazenyl)benzyl)carbamate (S24)

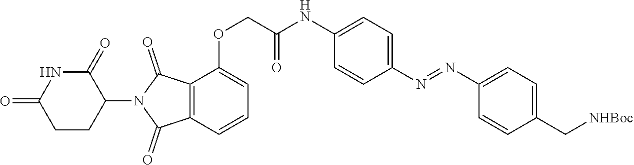 [SMILES: CC(C)(C)OC(=O)NCc1ccc(/N=N/c2ccc(NC(=O)COc3cccc4c3C(=O)N(C3CCC(=O)NC3=O)C4=O)cc2)cc1]

Thalidomide-4-hydroxyacetate (62) (20.0 mg, 60.2 μmol, 1.0 eq) and S23 (29.5 mg, 90.3 μmol, 1.5 eq) were dissolved in DMF (600 μL) followed by the addition of HATU (25.2 mg, 66.2 μmol, 1.1 eq) and DIPEA (21 μL, 120 μmol, 2.0 eq) at rt. The reaction was allowed to stir at rt overnight. The reaction was diluted with water, extracted 3 times with EtOAc, organics were combined, washed with bicarb and brine and dried over sodium sulfate. Concentration of organics and purification by column chromatography over SiO₂ using 8:1.5:0.5 DCM/EtOAc/MeOH as the mobile phase afforded S24 (37 mg, 57.8 μmol, 96%) as an orange amorphous solid. R_{f}=0.06 [Hexanes:EtOAc, 1:1]. ¹H NMR (400 MHz, Acetone-d₆) δ=10.03 (s, 1H), 9.84 (s, 1H), 8.05-7.93 (m, 4H), 7.93-7.82 (m, 3H), 7.62 (d, J=8.4 Hz, 1H), 7.58 (d, J=7.3 Hz, 1H), 7.51 (d, J=8.1 Hz, 2H), 5.25 (dd, J=12.5, 5.4 Hz, 1H), 5.00 (s, 2H), 4.39 (d, J=6.3 Hz, 2H), 3.09-2.91 (m, 1H), 2.92-2.78 (m, 3H), 2.39-2.20 (m, 1H), 1.46 (s, 9H) ppm. ¹³C NMR (101 MHz, Acetone) δ=172.7, 170.1, 167.7, 167.6, 166.8, 157.0, 155.8, 152.6, 149.8, 144.7, 142.0, 138.1, 134.4, 128.9, 124.7, 123.6, 122.1, 120.5, 119.3, 117.9, 79.1, 69.7, 50.5, 44.6, 32.1, 28.7, 23.4 ppm. HRMS (ESI): calcd. for C₃₃H₃₂N₆NaO₈⁺: 663.2174 m/z [M+Na]⁺; found: 663.2178 m/z [M+Na]⁺. LCMS T_{R}=4.505 min.

### 2-((S)-4-(4-chlorophenyl)-2,3,9-trimethyl-6H-thieno[3,2-f][1,2,4]triazolo[4,3-a][1,4]diazepin-6-yl)-N-(4-((E)-(4-(2-((2-(2,6-dioxopiperidin-3-yl)-1,3-dioxoisoindolin-4-yl)oxy)acetamido)phenyl)diazenyl)benzyl)acetamide (PHOTAC-I-11)

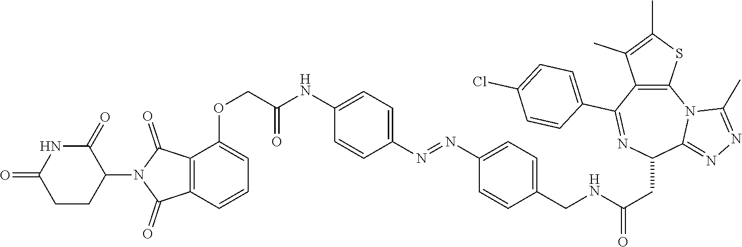

S24 (10.0 mg, 15.6 μmol, 1.0 eq) was dissolved in formic acid (1.6 mL), immediately turning the solution to a deep red color, and allowed to stir overnight at rt. After this period, the solvent was evaporated in vacuo to afford an orange amorphous solid. To this was added (+)-JQ1 free acid (6.6 mg, 16.4 μmol, 1.05 eq) in DMF (0.66 mL), HATU (8.9 mg, 23.4 μmol, 1.5 eq), followed by DIPEA (5 μL, 31.2 μmol, 2.0 eq) and the reaction was allowed to stir overnight at room temperature. The reaction was diluted with water, extracted 3 times with EtOAc, organics were combined, washed with bicarb and brine and dried over sodium sulfate. The organic layer was concentrated in vacuo and the residue was purified by semi-preparative reverse phase HPLC (50%→70% MeCN gradient+0.01% formic acid) affording PHOTAC-I-11 (6.0 mg, 6.5 μmol, 42%) as a yellow orange amorphous solid. R_{f}=0.29 [CH₂Cl₂:MeOH, 9:1]. ¹H NMR (400 MHz, Chloroform-d) δ=9.58 (d, J=8.8 Hz, 1H), 8.29 (d, J=18.0 Hz, 1H), 7.96 (qd, J=9.0, 2.8 Hz, 4H), 7.89-7.76 (m, 3H), 7.61 (dd, J=7.3, 1.4 Hz, 1H), 7.48 (d, J=8.2 Hz, 2H), 7.36-7.29 (m, 3H), 7.26 (d, J=8.8 Hz, 1H), 5.06 (dd, J=12.2, 5.2 Hz, 1H), 4.82 (d, J=4.0 Hz, 2H), 4.79-4.65 (m, 2H), 4.46 (dd, J=15.3, 5.3 Hz, 1H), 3.67-3.49 (m, 2H), 3.03-2.76 (m, 3H), 2.70 (s, 3H), 2.42 (s, 3H), 2.30-2.16 (m, 1H), 1.68 (s, 3H) ppm. ¹³C NMR (101 MHz, CDCl₃) δ=170.9, 170.9, 170.7, 168.0, 166.6, 166.5, 165.1, 164.3, 155.7, 154.4, 152.1, 150.1, 149.5, 141.5, 140.0, 137.4, 137.1, 136.5, 133.6, 132.3, 131.1, 130.6, 130.0, 128.9, 128.5, 124.2, 123.2, 120.0, 120.0, 118.0, 68.5, 54.6, 49.6, 43.4, 41.2, 39.4, 31.6, 22.8, 14.6, 13.3, 12.0 ppm. HRMS (APCI): calcd. for C₄₇H₄₃ClN₁₁O₇S⁺: 940.2751 m/z [M+NH₄]⁺; found: 940.2736 m/z [M+NH₄]⁺. LCMS T_{R}=4.422 min.

### tert-butyl (E)-(4-((4-nitrophenyl)diazenyl)phenethyl)carbamate (S25)

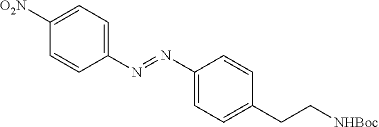 [SMILES: CC(C)(C)OC(=O)NCCc1ccc(/N=N/c2ccc([N+](=O)[O-])cc2)cc1]

The following procedure was carried out in two steps:

Oxone Oxidation: to a solution of 4-nitroaniline (462 mg, 3.3 mmol, 1.0 eq.) in CH₂Cl₂ (12.0 mL) was added a solution of oxone (2.1 g, 3.3 mmol, 1.0 eq.) in water (12.0 mL). The biphasic mixture was stirred vigorously under N₂ atmosphere. After 3 hours, phases were separated, and the aqueous phase was extracted with CH₂Cl₂. The organic phases were combined and then washed with 1 M HCl, sat. NaCl, dried over Na₂SO₄ and concentrated under reduced pressure to approximately 5 mL. The resulting yellow-black solution of nitrosobenzene in CH₂Cl₂ was carried on to the next step immediately.

Mills Reaction: to the nitrosobenzene solution in DCM, prepared as described above, was added sequentially tert-butyl (4-aminophenethyl)carbamate (791.0 mg, 3.3 mmol, 1.0 eq.) and glacial AcOH (0.96 mL, 17 mmol, 5.0 eq.). The reaction mixture was allowed to stir for 15 hours under N₂ atmosphere, after which time the reaction mixture was found to be an orange-black suspension. EtOAc was added and the organic phase was washed with 3× 1-M-NaOH, 2× sat. NaHCO₃, 2× sat. NaCl. The organic phase was then dried over Na₂SO₄ and concentrated under reduced pressure. Crude material was purified by flash column chromatography over SiO₂ using a gradient from 1%→5%→10% EtOAc in Hexanes as the eluent, affording S25 (724.0 mg, 1.955 mmol, 58%) as a crystalline red solid. R_{f}=0.23 [Hexanes:EtOAc, 9:1]. ¹H NMR (400 MHz, Chloroform-d) δ=8.36 (dd, J=8.8, 1.6 Hz, 2H), 8.00 (dd, J=8.7, 1.4 Hz, 2H), 7.91 (d, J=8.1 Hz, 2H), 7.37 (d, J=8.2 Hz, 2H), 4.62 (s, 1H), 3.43 (d, J=5.1 Hz, 2H), 2.90 (t, J=7.0 Hz, 2H), 1.44 (s, 9H) ppm. ¹³C NMR (101 MHz, CDCl₃) δ=155.9, 155.9, 151.3, 148.7, 144.3, 129.9, 124.8, 123.8, 123.5, 79.6, 41.7, 36.4, 28.5 ppm. HRMS (APCI): calcd. for C₁₄H₁₅N₄O₂⁺: 271.1195 m/z [M-Boc+H]⁺; found: 271.1190 m/z [M-Boc+H]⁺. LCMS T_{R}=4.858 min.

### tert-butyl (E)-(4-((4-aminophenyl)diazenyl)phenethyl)carbamate (S26)

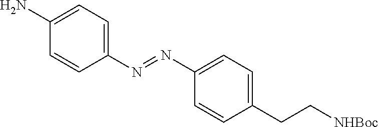 [SMILES: CC(C)(C)OC(=O)NCCc1ccc(/N=N/c2ccc(N)cc2)cc1]

To a solution of S25 (100.0 mg, 270 μmol, 1.0 eq.) in dioxane (4.0 mL) and water (0.4 mL) in a pressure tube was added Na₂S.9H₂O (195 mg, 810 μmol, 3.0 eq.). The reaction was sealed and heated to 85° C. After 1 hour, the reaction was diluted with water and the aqueous layer was extracted 3 times with EtOAc. The organics were combined, washed with brine, dried over sodium sulfate and concentrated in vacuo to afford an orange-red solid. The reaction was loaded onto isolute and purified by column chromatography over SiO₂ using a stepped gradient from 9:1→41:1 Hexanes/EtOAc as the eluent to afford S26 (77.0 mg, 226 μmol, 84%) as a pale orange solid. R_{f}=0.64 [Hexanes:EtOAc, 6:4]. ¹H NMR (400 MHz, Chloroform-d) δ=7.80 (d, J=4.5 Hz, 2H), 7.78 (d, J=3.9 Hz, 2H), 7.29 (d, J=8.1 Hz, 2H), 6.73 (d, J=8.8 Hz, 2H), 4.58 (s, 1H), 4.06 (s, 2H), 3.41 (d, J=6.7 Hz, 1H), 2.86 (t, J=7.0 Hz, 2H), 1.44 (s, 9H) ppm. ¹³C NMR (101 MHz, CDCl₃) δ=156.0, 151.8, 149.7, 145.7, 141.1, 129.5, 125.2, 122.7, 114.7, 79.4, 41.8, 36.1, 28.5 ppm. HRMS (APCI): calcd. for C₁₉H₂₅N₄O₂⁺: 341.1972 m/z [M+H]⁺; found: 341.1973 m/z [M+H]⁺. LCMS T_{R}=4.141 min.

### tert-butyl (E)-(4-((4-(2-((2-(2,6-dioxopiperidin-3-yl)-1,3-dioxoisoindolin-4-yl)oxy)acetamido)phenyl)diazenyl)phenethyl)carbamate (S27)

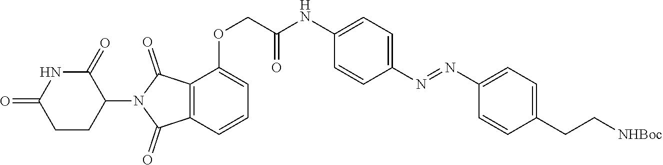 [SMILES: CC(C)(C)OC(=O)NCCc1ccc(/N=N/c2ccc(NC(=O)COc3cccc4c3C(=O)N(C3CCC(=O)NC3=O)C4=O)cc2)cc1]

Thalidomide-4-hydroxyacetate(62) (20.0 mg, 60.2 μmol, 1.0 eq.) and S26 (20.0 mg, 60.2 μmol, 1.0 eq.) were dissolved in DMF (600 μL) followed by the addition of TBTU (25 mg, 66.2 μmol, 1.10 eq.) and DIPEA (21 μL, 120.4 μmol, 2.0 eq.) at rt. The reaction was allowed to stir at room temperature overnight upon which the reaction was diluted with EtOAc, the organics were washed three times with equal portions of water, saturated sodium bicarbonate, and brine. The organic layer was dried over sodium sulfate and concentrated. The crude product was purified by column chromatography over SiO₂ using a gradient of 9:1 hexanes/EtOAc to 100% EtOAc as the eluent to afford product S27 (38.0 mg, 58.0 μmol, 96%) as an orange amorphous solid. R_{f}=0.29 [Hexanes:EtOAc, 6:4]. ¹H NMR (400 MHz, Chloroform-d) δ=9.54 (s, 1H), 8.41 (d, J=13.7 Hz, 1H), 7.92 (t, J=2.8 Hz, 4H), 7.83 (dd, J=8.3, 1.7 Hz, 2H), 7.80-7.70 (m, 1H), 7.63-7.51 (m, 1H), 7.32 (d, J=8.0 Hz, 2H), 7.23 (d, J=8.4 Hz, 1H), 5.04 (dd, J=12.3, 5.3 Hz, 1H), 4.78 (d, J=1.9 Hz, 2H), 4.60 (s, 1H), 3.41 (t, J=6.7 Hz, 2H), 2.97-2.78 (m, 6H), 2.25-2.15 (m, 1H), 1.44 (s, 9H) ppm. ¹³C NMR (101 MHz, CDCl₃) δ=171.0, 168.1, 166.6, 166.5, 165.1, 156.0, 154.4, 151.5, 149.5, 142.4, 139.9, 137.4, 133.6, 129.7, 124.1, 123.1, 120.0 (overlap of two signals), 118.6, 118.0, 79.5, 68.5, 49.6, 41.8, 36.2, 31.6, 28.5, 22.7 ppm. HRMS (ESI): calcd. for C₃₄H₃₅N₆O₈⁺: 655.2511 m/z [M+H]⁺; found: 655.2530 m/z [M+H]⁺. LCMS T_{R}=4.599 min.

### 2-((S)-4-(4-chlorophenyl)-2,3,9-trimethyl-6H-thieno[3,2-f][1,2,4]triazolo[4,3-a][1,4]diazepin-6-yl)-N-(4-((E)-(4-(2-((2-(2,6-dioxopiperidin-3-yl)-1,3-dioxoisoindolin-4-yl)oxy)acetamido)phenyl)diazenyl)phenethyl)acetamide (PHOTAC-I-12)

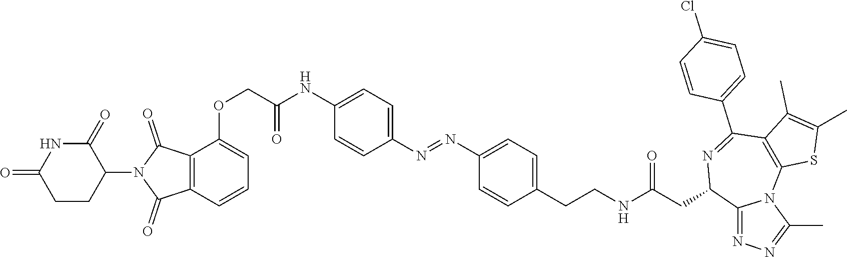 [SMILES: Cc1sc2c(c1C)C(c1ccc(Cl)cc1)=N[C@@H](CC(=O)NCCc1ccc(/N=N/c3ccc(NC(=O)COc4cccc5c4C(=O)N(C4CCC(=O)NC4=O)C5=O)cc3)cc1)c1nnc(C)n1-2]

S27 (5.0 mg, 9.0 μmol, 1.0 eq) was dissolved in formic acid (1.5 mL0 and allowed to stir overnight. After this period the reaction was concentrated and azeotroped to afford an orange residue. This crude material and (+)-JQ1-free acid (4.0 mg, 9.9 μmol, 1.1 eq) were dissolved in DMF (400 μL) followed by the addition of HATU (4.5 mg, 11.7 μmol, 1.3 eq) and DIPEA (2.0 μL, 13.5 μmol, 1.5 eq) at rt. The reaction was allowed to stir at rt overnight. The reaction was diluted with water and extracted 3 times with EtOAc. The organic layers were combined, washed with saturated sodium bicarbonate and brine and dried over sodium sulfate. Concentration of organics and purification by column chromatography over SiO₂ using 8:1.5:0.5 DCM/EtOAc/MeOH as the mobile phase afforded PHOTAC-I-12 (7.9 mg, 8.4 μmol, 94%) as an orange amorphous solid. R_{f}=0.17 [CH₂Cl₂:EtOAc:MeOH, 8:1.5:0.5]. ¹H NMR (400 MHz, Chloroform-d) δ=9.57 (d, J=2.4 Hz, 1H), 8.13 (d, J=2.8 Hz, 1H), 8.03-7.89 (m, 4H), 7.78 (dd, J=8.0, 4.0 Hz, 3H), 7.61 (d, J=2.0 Hz, 1H), 7.40-7.26 (m, 7H), 6.77 (s, 1H), 5.05 (dd, J=11.9, 5.0 Hz, 1H), 4.81 (s, 2H), 4.56 (t, J=6.9 Hz, 1H), 3.76-3.45 (m, 3H), 3.31 (dd, J=14.2, 5.8 Hz, 1H), 3.02-2.76 (m, 5H), 2.66 (s, 3H), 2.37 (s, 3H), 2.27-2.18 (m, 1H), 1.66 (s, 3H) ppm. ¹³C NMR (101 MHz, Chloroform-d) δ=170.8, 170.5, 167.9, 166.6, 166.5, 165.1, 164.2, 155.6, 154.4, 151.4, 150.0, 149.5, 142.3, 139.9, 137.4, 137.1, 136.4, 133.6, 132.1, 131.2, 131.1, 130.6, 130.0, 129.6, 128.9, 124.1, 123.0, 120.1 (overlap 2 peaks), 118.7, 118.1, 68.5, 54.6, 49.6, 40.5, 39.6, 35.6, 31.6, 22.8, 14.5, 13.2, 12.0 ppm. HRMS (ESI): calcd. C₄₈H₄₂ClN₁₀O₇S⁺: 937.2642 m/z [M+H]⁺; found: 937.2675 m/z [M+H]⁺. LCMS T_{R}=4.599 min.

### (Z)—N-(9-amino-11,12-dihydrodibenzo[c,g][1,2]diazocin-2-yl)-2-((2-(2,6-dioxopiperidin-3-yl)-1,3-dioxoisoindolin-4-yl)oxy)acetamide (S28)

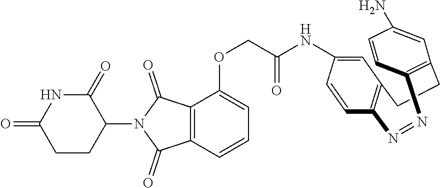

Thalidomide-4-hydroxyacetate (20.0 mg, 60 μmol, 1.0 eq.) and (Z)-11,12-dihydrodibenzo[c,g][1,2]diazocine-2,9-diamine (21.5 mg, 90 μmol, 1.5 eq.) were dissolved in DMF (0.6 mL) followed by the addition of TBTU (21.3 mg, 66 μmol, 1.1 eq.) and DIPEA (21 μL, 120 μmol, 2.0 eq.) at rt. The reaction was allowed to stir at rt overnight. The reaction was diluted with CH₂Cl₂ and successively washed with water, saturated sodium bicarbonate, and brine. The organic layer was dried over sodium sulfate and concentrated. The residue was purified by column chromatography over silica using a 1% to 3% MeOH in CH₂Cl₂ as the eluent to afford S28 (17.0 mg, 31 μmol, 51%) as an amorphous yellow solid. This product was contaminated with an unknown impurity and used in the next step. R_{f}=0.08 [CH₂Cl₂:MeOH, 95:5]. ¹H NMR (400 MHz, DMSO-d₆) δ 11.12 (s, 1H), 10.10 (s, 1H), 7.80 (ddd, J=8.5, 7.3, 4.2 Hz, 1H), 7.50 (d, J=7.2 Hz, 1H), 7.47-7.38 (m, 2H), 7.38-7.30 (m, 1H), 6.81 (dd, J=30.2, 8.4 Hz, 1H), 6.63-6.51 (m, 1H), 6.36 (dd, J=8.4, 2.3 Hz, 1H), 6.20 (d, J=2.3 Hz, 1H), 5.13 (q, J=6.5, 5.5 Hz, 2H), 4.96 (d, J=3.2 Hz, 2H), 2.90 (s, 2H), 2.76-2.55 (m, 6H), 2.08-1.92 (m, 1H) ppm. ¹³C NMR (101 MHz, DMSO) δ=172.8, 169.9, 166.7, 165.6, 165.5, 162.3, 155.2, 151.4, 147.9, 145.4, 136.9, 136.7, 133.0, 129.5, 128.5, 121.0, 120.5, 119.5, 117.4, 116.7, 116.0, 113.6, 111.9, 67.4, 48.8, 31.6, 31.2, 30.9, 22.0 ppm. HRMS (APCI): calcd. C₂₉H₂₅N₆O₆⁺: 553.1836 m/z [M+H]⁺; found: 553.1828 m/z [M+H]⁺. LCMS T_{R}=2.931 min.

### 2-((S)-4-(4-chlorophenyl)-2,3,9-trimethyl-6H-thieno[3,2-f][1,2,4]triazolo[4,3-a][1,4]diazepin-6-yl)-N—((Z)-9-(2-((2-(2,6-dioxopiperidin-3-yl)-1,3-dioxoisoindolin-4-yl)oxy)acetamido)-11,12-dihydrodibenzo[c,g][1,2]diazocin-2-yl)acetamide (PHOTAC-I-13)

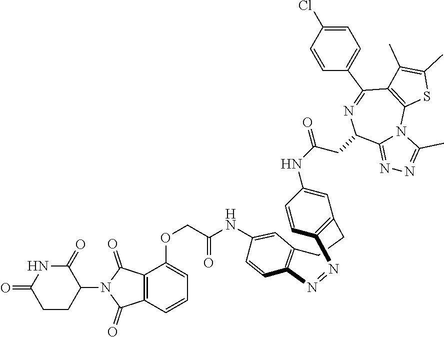

To a solution of S28 (5.0 mg, 9.0 μmol, 1.0 eq.) and (+)-JQ1 free acid (4.0 mg, 10 μmol, 1.1 eq.) in DCE (1.8 mL) was added TBTU (3.8 mg, 11.8 μmol, 1.3 eq.) followed by DIPEA (1.8 mg, 2.0 μL, 13.6 μmol, 1.5 eq.) at room temperature. The reaction was allowed to stir overnight upon which the reaction was diluted with EtOAc, washed with equal portions of water, saturated sodium bicarbonate, and brine. The organic layer was dried over sodium sulfate and concentrated under reduced pressure. The residue was purified by column chromatography over SiO₂ using a gradient of 0%→10% MeOH in CH₂Cl₂ as the eluent to afford PHOTAC-I-13 (5.9 mg, 5.9 μmol, 65%) as a amorphous yellow solid. R_{f}=0.39 [CH₂Cl₂: MeOH, 95:5]. ¹H NMR (400 MHz, Chloroform-d) δ=9.23 (d, J=122.5 Hz, 2H), 7.84-7.74 (m, 1H), 7.75-7.63 (m, 1H), 7.60 (dd, J=7.4, 3.4 Hz, 1H), 7.42 (dd, J=8.6, 2.6 Hz, 2H), 7.38-7.32 (m, 2H), 7.25 (dd, J=8.5, 6.2 Hz, 1H), 6.87 (d, J=8.6 Hz, 1H), 6.81 (d, J=8.5 Hz, 1H), 5.12-4.93 (m, 1H), 4.73 (d, J=8.6 Hz, 2H), 4.61 (dd, J=9.2, 4.7 Hz, 1H), 3.79 (ddd, J=22.4, 14.1, 9.1 Hz, 1H), 3.44 (ddd, J=19.4, 14.0, 4.8 Hz, 1H), 3.04-2.75 (m, 7H), 2.68 (d, J=4.3 Hz, 3H), 2.64 (d, J=0.8 Hz, 5H), 2.42 (d, J=2.4 Hz, 3H), 2.31-2.15 (m, 1H), 1.72-1.60 (m, 3H) ppm. ¹³C NMR (101 MHz, Chloroform-d) δ=168.5, 168.3, 166.5, 164.8, 164.7, 155.4, 154.4, 152.1, 150.1, 137.2, 136.2, 133.5, 131.3, 131.0, 130.5, 129.9, 129.9, 129.2, 128.8, 120.5, 120.2, 118.7, 118.0, 117.8, 68.8, 68.5, 54.6, 54.4, 49.4, 41.0, 31.5, 31.5, 22.6, 14.4, 14.4, 13.1, 11.7 ppm. HRMS (APCI): calcd. C₄₈H₄₀ClN₁₀O₇S⁺: 935.2491 m/z [M+H]⁺; found: 935.2478 m/z [M+H]⁺.

### (E)-2-(2,6-dioxopiperidin-3-yl)-5-((4-hydroxy-3,5-dimethoxyphenyl)diazenyl)isoindoline-1,3-dione (S29)

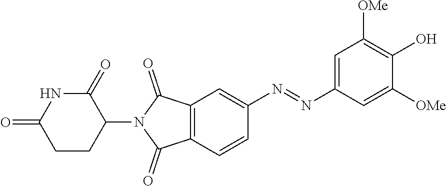 [SMILES: COc1cc(/N=N/c2ccc3c(c2)C(=O)N(C2CCC(=O)NC2=O)C3=O)cc(OC)c1O]

To a solution of 5-aminothalidomide (200 mg, 0.73 μmol, 1.0 eq) and NaNO₂ (424 μl, 848 μmol, 1.16 equiv) in acetone/water (4:1, 8 mL) was added 4 equiv. of HCl (4.0 M in 1,4-dioxane, 732 μL, 2.9 mmol, 4.0 equiv) at 0° C. After stirring for 1 h, the solution was added in a dropwise fashion to a mixture of 2,6-dimethoxyphenol, (135 mg, 0.88 mmol, 1.2 equiv), NaHCO₃ (1.5 g, 18.1 mmol, 24.7 equiv), Na₂CO₃ (3.7 g, 34.5 mmol, 47.2 equiv) in water/MeOH (5:2, 28 mL) at 0° C. and allowed to stir for an additional hour. After this time period, the reaction was quenched with sat. NH₄Cl and extracted with EtOAc. The organic layers were combined and washed with brine and concentrated under reduced pressure. The residue was purified by column chromatography over SiO₂ using 4:6 Hexanes/EtOAc as the eluent to afford S29 (49.0 mg, 112 μmol, 15%) as a red solid. R_{f}=0.23 [Hexanes:EtOAc, 4:6]. ¹H NMR (400 MHz, Acetone-d₆) δ=9.96 (s, 1H), 8.31 (dd, J=5.3, 2.6 Hz, 1H), 8.21 (s, 1H), 8.07 (d, J=7.9 Hz, 1H), 7.43 (s, 2H), 5.21 (dd, J=12.6, 5.5 Hz, 1H), 3.98 (s, 6H), 2.99 (m, 1H), 2.88-2.77 (m, 2H), 2.32-2.25 (m, 1H). ¹³C NMR (101 MHz, Acetone) δ=172.6, 169.9, 167.5, 167.5, 157.6, 149.1, 145.8, 142.0, 134.2, 133.0, 130.6, 125.4, 116.0, 102.6, 56.7, 50.5, 32.0, 23.2. HRMS (APCI): calcd. for C₂₁H₁₉N₄O₇⁺: 439.1248 m/z [M+H]⁺; found: 439.1251 m/z [M+H]⁺. LCMS T_{R}=3.180 min.

### tert-butyl (E)-2-(4-((2-(2,6-dioxopiperidin-3-yl)-1,3-dioxoisoindolin-5-yl)diazenyl)-2,6-dimethoxyphenoxy)acetate (S30)

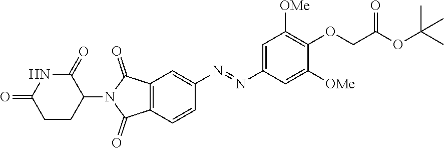 [SMILES: COc1cc(/N=N/c2ccc3c(c2)C(=O)N(C2CCC(=O)NC2=O)C3=O)cc(OC)c1OCC(=O)OC(C)(C)C]

S29 (35.0 mg, 78.8 μmol, 1.0 eq) was dissolved in DMF (0.8 mL) at room temperature. To this was added K2CO₃ (16.6 mg, 0.12 mmol, 1.5 equiv), immediately turning the solution a blue-black color, followed by the addition of tert-butyl bromoacetate (16.4 mg, 83.8 μmol, 1.05 eq). The reaction was allowed to stir at room temperature for 2 hours upon which the reaction was quenched with saturated aqueous NH₄Cl. The aqueous layer was extracted three times with EtOAc, the organics were combined, washed with brine, dried over sodium sulfate and concentrated. The residue was purified by column chromatography over SiO₂ using a gradient of 0→30% EtOAc in DCM to afford S30 (17.0 mg, 30.8 μmol, 39%) as an orange film. R_{f}=0.47 [CH₂Cl₂:EtOAc, 7:3]. ¹H NMR (400 MHz, Chloroform-d) δ=8.37 (d, J=1.6 Hz, 1H), 8.29 (dd, J=7.9, 1.7 Hz, 1H), 8.06 (d, J=8.0 Hz, 1H), 8.00 (s, 1H), 7.34 (s, 2H), 5.05 (dd, J=12.4, 5.4 Hz, 1H), 4.74 (s, 2H), 3.99 (s, 6H), 3.02-2.70 (m, 4H), 2.31-2.14 (m, 1H), 1.51 (s, 9H) ppm. ¹³C NMR (101 MHz, CDCl₃) δ=170.7, 168.3, 167.8, 166.8, 166.7, 156.9, 152.8, 147.9, 140.6, 133.2, 132.3, 130.1, 129.8, 125.2, 125.0, 119.4, 117.0, 101.5, 81.9, 70.0, 56.5, 49.7, 31.6, 29.9, 28.3, 22.8 ppm. HRMS (APCI): calcd. for C₂₃H₂₁N₄O₉⁺: 497.1303 m/z [M-tBu+H]⁺; found: 497.1292 m/z [M-tBu+H]⁺. LCMS T_{R}=3.383 min.

### tert-butyl (E)-(2-(2-(4-((2-(2,6-dioxopiperidin-3-yl)-1,3-dioxoisoindolin-5-yl)diazenyl)-2,6-dimethoxyphenoxy)acetamido)ethyl)carbamate (S31)

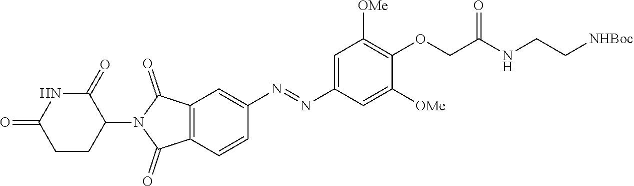 [SMILES: COc1cc(/N=N/c2ccc3c(c2)C(=O)N(C2CCC(=O)NC2=O)C3=O)cc(OC)c1OCC(=O)NCCNC(=O)OC(C)(C)C]

S30 (8.0 mg, 14.5 μmol, 1.0 eq.) was dissolved in formic acid (1.4 mL), immediately turning the solution to a deep red color, and allowed to stir overnight at rt. After this period, the solvent was evaporated in vacuo to afford an orange-red amorphous solid. To this was added N-Boc-ethylene diamine (2.8 mg, 17.4 μmol, 1.2 eq.) in DMF (1.4 mL), HATU (8.3 mg, 21.7 μmol, 1.5 eq.), followed by DIPEA (5 μL, 3.7 mg, 28.7 μmol, 2.0 eq.) and the reaction was allowed to stir overnight at room temperature. The reaction was diluted with water, extracted 3 times with EtOAc, organics were combined, washed with bicarb and brine and dried over sodium sulfate. The organic layer was concentrated in vacuo and the residue was purified column chromatography over SiO₂ using 0%→3% MeOH in CH₂Cl₂ as the eluent to afford S31 (5.7 mg, 8.9 μmol, 62%) as an orange film. R_{f}=0.09 [CH₂Cl₂:MeOH, 97:3]. ¹H NMR (400 MHz, Chloroform-d) δ=8.35 (d, J=1.5 Hz, 1H), 8.28 (dd, J=7.9, 1.7 Hz, 1H), 8.17 (s, 1H), 8.04 (d, J=7.9 Hz, 1H), 7.91-7.83 (m, 1H), 7.33 f(s, 2H), 5.03 (dd, J=12.2, 5.5 Hz, 1H), 4.90 (s, 1H), 4.63 (s, 2H), 4.01 (s, 6H), 3.49 (d, J=6.0 Hz, 2H), 3.38-3.24 (m, 2H), 3.02-2.63 (m, 3H), 2.28-2.09 (m, 1H), 1.43 (s, 9H) ppm. ¹³C NMR (101 MHz, CDCl₃) δ=170.8, 170.3, 167.9, 166.7, 166.7, 156.7, 156.2, 152.8, 148.6, 140.4, 133.2, 132.6, 130.2, 125.0, 116.9, 101.3, 79.7, 72.8, 56.5, 49.7, 40.9, 39.2, 31.6, 28.5, 22.8 ppm. HRMS (APCI): calcd. for C₂₅H₂₇N₆O₈⁺: 539.1885 m/z [M-Boc+H]⁺; found: 539.1873 m/z [M-Boc+H]⁺. LCMS T_{R}=3.953 min.

### 2-((S)-4-(4-chlorophenyl)-2,3,9-trimethyl-6H-thieno[3,2-f][1,2,4]triazolo[4,3-a][1,4]diazepin-6-yl)-N-(2-(2-(4-((E)-(2-(2,6-dioxopiperidin-3-yl)-1,3-dioxoisoindolin-5-yl)diazenyl)-2,6-dimethoxyphenoxy)acetamido)ethyl)acetamide (PHOTAC-I-9)

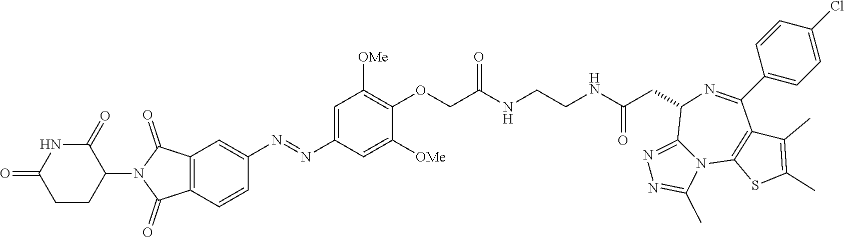

S31 (5.7 mg, 8.9 μmol, 1.0 equiv) was dissolved in formic acid and allowed to stir at room temperature for 30 min. After this period, the reaction was concentrated in vacuo and azeotroped. The crude residue was used immediately without further purification. This crude material and (+)-JQ1-free acid (3.9 mg, 9.8 μmol, 1.1 eq) were dissolved in DMF (400 μL) followed by the addition HATU (3.7 mg, 9.8 μmol, 1.1 eq) and DIPEA (2.3 μL, 17.9 μmol, 2.0 eq) at rt. The reaction was allowed to stir at rt overnight. The reaction was diluted with water and extracted 3 times with EtOAc. The organic layers were combined, washed with saturated sodium bicarbonate and brine and dried over sodium sulfate. Concentration of organics and purification by column chromatography over SiO₂ using 0%→10% MeOH in DCM as the mobile phase afforded PHOTAC-I-9 (4.1 mg, 4.4 μmol, 50%) as an orange amorphous solid. R_{f}=0.23 [CH₂Cl₂:MeOH, 9:1]. ¹H NMR (400 MHz, Chloroform-d) δ=8.32 (d, J=1.5 Hz, 1H), 8.26 (dd, J=7.9, 1.6 Hz, 1H), 8.16 (s, 1H), 8.02 (d, J=7.9 Hz, 1H), 7.90 (d, J=6.4 Hz, 1H), 7.42 (d, J=8.2 Hz, 2H), 7.33 (d, J=8.6 Hz, 3H), 7.31 (s, 2H), 5.03 (dd, J=12.3, 5.4 Hz, 1H), 4.69 (t, J=6.8 Hz, 1H), 4.62 (s, 2H), 3.99 (s, 6H), 3.65-3.35 (m, 6H), 3.02-2.71 (m, 3H), 2.69 (s, 3H), 2.40 (s, 3H), 2.34 (t, J=7.5 Hz, 2H), 1.67 (s, 3H), 1.67-1.55 (m, 1H) ppm. ¹³C NMR (101 MHz, CDCl₃) δ=176.9, 171.0, 170.8, 170.5, 167.9, 166.8, 166.7, 164.7, 156.7, 155.4, 152.8, 150.3, 148.6, 140.5, 137.5, 136.0, 135.9, 133.1, 132.5, 131.3, 130.2, 130.2, 129.0, 129.0, 125.0, 117.0, 101.4, 72.8, 56.6, 53.6, 49.7, 40.0, 38.8, 33.7, 32.1, 31.6, 24.9, 22.8, 22.8, 14.6, 13.3, 11.9 ppm. HRMS (APCI): calcd. for C₄₄H₄₂ClN₁₀O₉S⁺: 921.2545 m/z [M+H]⁺; found: 921.2519 m/z [M+H]⁺. LCMS T_{R}=4.089 min.

### Dimethyl 4-aminophthalate (MB-15)

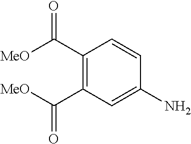 [SMILES: COC(=O)c1ccc(N)cc1C(=O)OC]

1,2-Dimethyl-4-nitrophthalate (1.00 g, 4.18 mmol, 1 eq.) and Pd/C (390 mg, 0.37 mmol, 0.1 eq.) were dissolved in dry MeOH (12 mL) under nitrogen. The flask was then charged with hydrogen gas and the reaction mixture was stirred for 18 h. It was filtered by using Celite and washed with MeOH. The reaction was concentrated under reduced pressure and dried under high vacuum. MB-15 was obtained as a yellow oil (804.0 mg, 3.843 mmol, 92%). R_{f}=0.19 [Hx:EtOAc, 6:3]. ¹H NMR (400 MHz, DMSO-d₆) δ=7.57 (d, J=8.5 Hz, 1H), 6.63 (dd, J=8.5, 2.3 Hz, 1H), 6.59 (d, J=2.2 Hz, 1H), 6.17 (s, 2H), 3.76 (s, 3H), 3.71 (s, 3H) ppm. HRMS (ESI): calcd. for C₁₀H₁₂NO₄⁺: 210.0670 m/z [M+H]⁺; found: 210.0770 m/z [M+H]⁺. LCMS (ESI): tᵣₑₜ=2.34 min. 210 m/z [M+H]⁺. The analytical data matched those previously described.

### Dimethyl 4-amino-5-iodophthalate (MB-16)

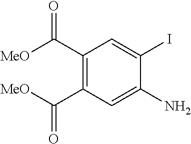 [SMILES: COC(=O)c1cc(N)c(I)cc1C(=O)OC]

MB-15 (1395.5 mg, 6.671 mmol, 1.0 eq.) and N-Iodosuccinimide (1508.2 mg, 6.704 mmol, 1.005 eq.) were dissolved in DMSO (34 mL) and stirred for 72 h at room temperature. The reaction was diluted with EtOAc (60 mL), separated against H₂O (60 mL), extracted with EtOAc (2×60 mL) and the combined organic phases were washed twice with brine (2×50 mL), dried over Na₂SO₄ and concentrated under reduced pressure. Purification of the resulting crude product by flash column chromatography (Hx/Ea gradient, 0→100% Ea) gave MB-16 (1314.5 mg, 3.923 mmol, 59%) as a red-orange oil. R_{f}=0.5 [CH₂Cl₂:MeOH, 19:1]. ¹H NMR (400 MHz, Chloroform-d) δ=8.20 (s, 1H), 6.79 (s, 1H), 4.57 (s, 2H), 3.89 (s, 3H), 3.84 (s, 3H) ppm. LCMS (ESI): tᵣₑₜ=3.27 min. 336 m/z [M+H]⁺.

### Tert-butyl 4-iodo-3-nitrobenzoate (MB-18)

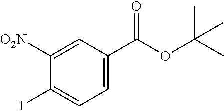 [SMILES: CC(C)(C)OC(=O)c1ccc(I)c([N+](=O)[O-])c1]

MB-18 was prepared similar to the previously described method^{[35]}: Concentrated sulfuric acid (0.5 mL, 10 mmol, 1 eq.) was added to a stirred suspension of magnesium sulfate (4.9 g, 40 mmol, 4 eq.) in dry CH₂Cl₂ (30 mL). After 20 minutes of stirring, 4-iodo-3-nitrobenzoic acid (2.93 g, 10 mmol, 1 eq.) was added to the mixture and tert-butanol (4.7 mL, 50 mmol, 5 eq.) was added last. After 20 h of stirring the reaction was quenched with saturated sodium bicarbonate solution (75 mL). The organic phase was separated, washed with brine (2×50 mL), dried over Na₂SO₄ and was concentrated under reduced pressure. MB-18 (2308 mg, 6.611 mmol, 66%) was obtained as a light brown solid. R_{f}=0.47 [CH₂Cl₂:MeOH, 9:1]. LCMS (ESI): tᵣₑₜ=4.71 min. 373 m/z [M+Na]⁺.

### Tert-butyl 3-nitro-4-((trimethylsilyl)ethynyl)benzoate (MB-22)

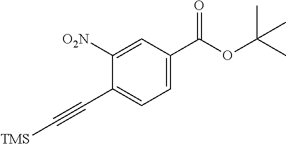 [SMILES: CC(C)(C)OC(=O)c1ccc(C#C[Si](C)(C)C)c([N+](=O)[O-])c1]

MB-18 (1745 mg, 5.000 mmol, 1 eq.), CuI (47.6 mg, 0.25 mmol, 0.05 eq.) and PdCl₂(PPh₃)₂ (175.5 mg, 0.250 mmol, 0.050 eq.) were suspended in dry THF (25 mL) under nitrogen. After addition of NEt₃ (2.1 mL, 15 mmol, 3 eq.) and TMS acetylene (0.85 mL, 6.00 mmol, 1.2 eq.) the reaction mixture was stirred for 3 h. The reaction was diluted with Et₂O (30 mL), filtered through Celite and concentrated under reduced pressure. Purification of the resulting crude product by flash column chromatography (Hx/EA gradient, 0→100% EA) gave MB-22 (1372.4 mg, 4.296 mmol, 86%) as a yellow solid. R_{f}=0.60 [Hx:EA, 10:1]. ¹H NMR (400 MHz, Chloroform-d) δ=8.41 (s, 1H), 8.01-7.97 (m, 1H), 7.55 (d, J=8.1 Hz, 1H), 1.47 (s, 9H), 0.15 (s, 9H) ppm. ¹³C NMR (101 MHz, CDCl₃) δ=163.21, 150.23, 135.18, 133.02, 132.66, 125.49, 121.91, 107.27, 98.96, 82.96, 28.23, −0.32 ppm. HRMS (ESI): calcd. for C₁₄H₁₅Br₂N₂⁺: 368.9602 m/z [M+H]⁺; found: 368.9606 m/z [M+H]⁺. LCMS (ESI, 50 to 100): tᵣₑₜ=4.13 min. no ions observed.

### Tert-butyl 4-ethynyl-3-nitrobenzoate (MB-27)

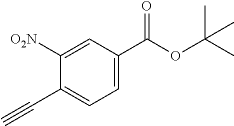 [SMILES: C#Cc1ccc(C(=O)OC(C)(C)C)cc1[N+](=O)[O-]]

MB-22 (1300 mg, 4.07 mmol, 1 eq.) was dissolved in dry THF. After addition of Et₃N.HF (328 mg, 2.035 mmol, 0.5 eq.) the mixture was stirred for 15 minutes. The reaction was then diluted with Et₂O (20 mL) and washed with sat. aq. NaHCO₃ (20 mL). The layers were separated and the aqueous phase was extracted twice with Et₂O (2×20 mL). The combined organic layers were dried over Na₂SO₄ and concentrated under reduced pressure. Purification of the resulting crude product by flash column chromatography (Hx/EtOAc gradient, 0→100% EtOAc) gave MB-27 (836.6 mg, 3.384 mmol, 83%) as a yellow solid. R_{f}=0.38 [Hx:EA, 9:1]. ¹H NMR (400 MHz, Chloroform-0 6=8.60-8.57 (m, 1H), 8.19-8.14 (m, 1H), 7.74 (d, J=8.1 Hz, 1H), 3.66 (s, 1H), 1.61 (s, 9H) ppm. ¹³C NMR (100 MHz, CDCl₃) δ=163.05, 150.40, 135.63, 133.33, 133.21, 125.57, 120.96, 87.89, 83.12, 78.28, 28.24 ppm. HRMS (ESI): calcd. for C₁₃H₁₃NNaO₄⁺: 270.0737 m/z [M+Na]⁺; found: 270.0748 m/z [M+Na]⁺. LCMS (ESI, 50 to 100): tᵣₑₜ=2.04 min. no ions observed.

### Dimethyl 4-amino-5-((4-(tert-butoxycarbonyl)-2-nitrophenyl)ethynyl)phthalate (MB-35)

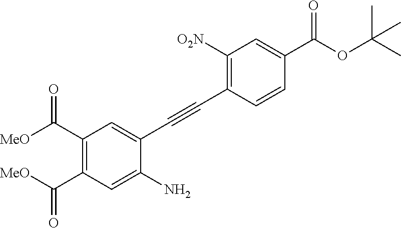 [SMILES: COC(=O)c1cc(N)c(C#Cc2ccc(C(=O)OC(C)(C)C)cc2[N+](=O)[O-])cc1C(=O)OC]

MB-16 (1084.2 mg, 3.236 mmol, 1 eq.), MB-27 (800 mg, 3.236 mmol, 1 eq.), PdCl₂(PPh₃)₂ (113.6 mg, 0.162 mmol, 0.05 eq.) and CuI (30.8 mg, 0.162 mmol, 0.05 eq.) were suspended in dry THF (10 mL) under nitrogen. NEt₃ (982.2 mg, 9.707 mmol, 3 eq., 1.3 mL) were added to the mixture and stirred for 24 h at room temperature. TMS acetylene (63.5 mg, 0.647 mmol, 0.02 eq., 0.09 mL) were added and the reaction was stirred for additional 3 hours. The reaction was then diluted with EtOAc (30 mL) and filtered by using Celite. Purification of the resulting crude product by flash column chromatography (Hx/EA gradient, 0→100% EA, with constant 5% CH₂Cl₂) gave MB-35 (426.0 mg, 0.937 mmol, 29%) as a light brownish/yellow solid. R_{f}=0.36 [Hx:EA:CH₂Cl₂, 5:1:1]. ¹H NMR (400 MHz, Chloroform-d) δ=8.74 (s, 1H), 8.23 (d, J=8.1 Hz, 1H), 8.02 (s, 1H), 7.79 (d, J=8.1 Hz, 1H), 6.81 (s, 1H), 5.24 (s, 2H), 3.92 (s, 3H), 3.86 (s, 3H), 1.63 (s, 9H) ppm. ¹³C NMR (100 MHz, CDCl₃) δ 169.02, 165.80, 162.97, 152.28, 148.26, 137.50, 135.46, 134.45, 133.67, 132.45, 126.17, 122.00, 117.60, 113.18, 106.35, 95.75, 92.00, 82.97, 52.82, 52.26, 28.12 ppm. HRMS (ESI): calcd. for C₂₃H₂₂N₂NaO₈⁺: 477.1268 m/z [M+Na]⁺; found: 477.1252 m/z [M+Na]⁺. LCMS (ESI): tᵣₑₜ=4.88 min. 455 m/z [M+H]⁺.

### Dimethyl 4-amino-5-((4-(tert-butoxycarbonyl)-2-nitrophenyl)ethynyl)phthalate (MB-61)

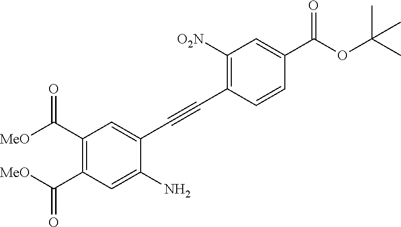 [SMILES: COC(=O)c1cc(N)c(C#Cc2ccc(C(=O)OC(C)(C)C)cc2[N+](=O)[O-])cc1C(=O)OC]

Alternatively, MB-18 (389.0 mg, 1.114 mmol, 1 eq.), MB-59 (519.7 mg, 2.228 mmol, 2 eq.), PdCl₂(PPh₃)₂ (46.0 mg, 0.066 mmol, 0.059 eq.) and CuI (25.0 mg, 0.13 mmol, 0.12 eq.) were suspended in dry THF (10 mL) under nitrogen. NEt₃ (338.2 mg, 3.343 mmol, 3 eq., 0.47 mL) were added to the mixture and stirred for 12 h at room temperature. The reaction was then diluted with EtOAc (30 mL) and filtered by using Celite. Purification of the resulting crude product by flash column chromatography (Hx/EA gradient, 0→100% EA, with constant 5% CH₂Cl₂) gave MB-61 (430.0 mg, 0.946 mmol, 85%) as a light brownish/yellow solid. R_{f}=0.36 [Hx:EA:CH₂Cl₂, 5:1:1]. ¹H NMR (400 MHz, Chloroform-d) δ=8.74 (s, 1H), 8.23 (d, J=8.1 Hz, 1H), 8.02 (s, 1H), 7.79 (d, J=8.1 Hz, 1H), 6.81 (s, 1H), 5.24 (s, 2H), 3.92 (s, 3H), 3.86 (s, 3H), 1.63 (s, 9H) ppm. ¹³C NMR (100 MHz, CDCl₃) δ 169.02, 165.80, 162.97, 152.28, 148.26, 137.50, 135.46, 134.45, 133.67, 132.45, 126.17, 122.00, 117.60, 113.18, 106.35, 95.75, 92.00, 82.97, 52.82, 52.26, 28.12 ppm. HRMS (ESI): calcd. for C₂₃H₂₂N₂NaO₈⁺: 477.1268 m/z [M+Na]⁺; found: 477.1252 m/z [M+Na]⁺. LCMS (ESI): tᵣₑₜ=4.95 min. 455 m/z [M+H]⁺.

### Dimethyl 4-amino-5-(2-amino-4-(tert-butoxycarbonyl)phenethyl)phthalate (MB-48)

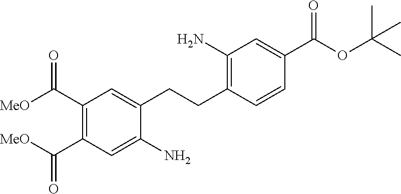 [SMILES: COC(=O)c1cc(N)c(CCc2ccc(C(=O)OC(C)(C)C)cc2N)cc1C(=O)OC]

MB-35 (260.0 mg, 0.57 mmol, 1 eq.) and Pd/C (60.9 mg, 0.057 mmol, 0.1 eq.) were dissolved in dry MeOH (12 ml) under nitrogen. The flask was then charged with hydrogen gas and the reaction mixture was stirred for 24 h. It was filtered by using Celite and washed with MeOH, concentrated under reduced pressure and dried on high vacuum. Pd/C (60.9 mg, 0.057 mmol, 1 eq.) was added again into the round bottom flask and dissolved in dry MeOH (12 mL). The flask was then charged with hydrogen gas again. After additional 48 h of stirring the reaction was filtered by using Celite, washed with MeOH and concentrated under reduced pressure. Purification of the resulting crude product by flash column chromatography (Hx/EA gradient, 0→100% Ea+5% MeOH/DCM) gave MB-48 (161.0 mg, 0.376 mmol, 66%) as a yellow oil. R_{f}=0.33 [Hx:EA, 1:1]. ¹H NMR (400 MHz, Chloroform-d) δ=7.62 (s, 1H), 7.37-7.33 (m, 1H), 7.31 (s, 1H), 7.02 (d, J=7.8 Hz, 1H), 6.73 (s, 1H), 3.88 (s, 3H), 3.84 (s, 3H), 2.90-2.73 (m, 4H), 1.58 (s, 9H) ppm. ¹³C NMR (100 MHz, CDCl₃) δ=169.63, 167.09, 165.95, 148.08, 144.01, 134.15, 131.74, 131.48, 130.39, 129.65, 126.51, 120.60, 119.08, 116.95, 114.55, 80.95, 52.75, 52.27, 31.07, 30.02, 28.34 ppm. HRMS (ESI): calcd. for C₂₃H₂₉N₂O₆⁺: 429.2020 m/z [M+H]⁺; found: 429.2033 m/z [M+H]⁺. LCMS (ESI): tᵣₑₜ=4.04 min. 427 m/z [M−H]⁻.

### 8-(tert-butyl) 2,3-dimethyl (Z)-11,12-dihydrodibenzo[c,g][1,2]diazocine-2,3,8-tricarboxylate (MB-50)

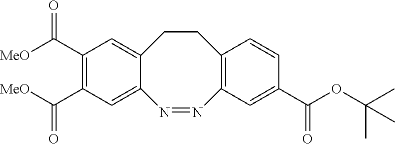 [SMILES: COC(=O)c1cc2c(cc1C(=O)OC)/N=N\c1cc(C(=O)OC(C)(C)C)ccc1CC2]

MB-48 (138.0 mg, 0.322 mmol, 1 eq.) was dissolved in a mixture of CH₂Cl₂ (8 mL) and AcOH (8 mL). Peracetic acid (0.11 mL) was diluted in 2 mL AcOH and added dropwise over 24 h at room temperature in the dark using a syringe pump. The reaction was stirred for additional 12 hours and the volatiles were removed in vacuo. The residue was taken up in CH₂Cl₂ (10 mL), separated against NaHCO₃ (10 mL) and extracted with CH₂Cl₂ (2×10 mL). The combined organic phase was washed with brine (2×30 mL), dried over Na₂SO₄ and the solvent was removed in vacuo. Purification of the resulting crude product by flash column chromatography (Hx/Ea gradient, 0→100% Ea) gave MB-50 (19.8 mg, 0.047 mmol, 15%) as a yellow oil. R_{f}=0.15 [Hx:EA, 7:3]. ¹H NMR (400 MHz, Chloroform-d) δ=7.67 (dd, J=8.0, 1.7 Hz, 1H), 7.49 (s, 1H), 7.36 (s, 1H), 7.24 (s, 1H), 7.05 (d, J=8.0 Hz, 1H), 3.86 (s, 3H), 3.84 (s, 3H), 3.12-2.95 (m, 2H), 2.93-2.78 (m, 2H), 1.56 (s, 9H) ppm. ¹³C NMR (100 MHz, CDCl₃) δ=167.42, 166.96, 164.62, 156.73, 154.88, 131.75, 131.46, 131.46, 130.91, 130.84, 130.78, 129.91, 128.82, 120.34, 120.30, 81.73, 52.92, 52.87, 31.76, 31.35, 28.25 ppm. HRMS (APCI): calcd. for C₂₃H₂₅N₂O₆⁺: 425.1707 m/z [M+H]⁺; found: 425.1705 m/z [M+H]⁺; LCMS (ESI): tᵣₑₜ=4.61 min. 425 m/z [M+H]⁺.

### Dimethyl 4-amino-5-((trimethylsilyl)ethynyl)phthalate (MB-56)

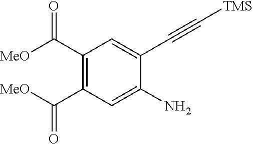 [SMILES: COC(=O)c1cc(N)c(C#C[Si](C)(C)C)cc1C(=O)OC]

MB-16 (1000 mg, 2.984 mmol, 1 eq.), CuI (28.4 mg, 0.149 mmol, 0.05 eq.) and PdCl₂(PPH₃)₂ (104.7 mg, 0.149 mmol, 0.05 eq.) were suspended in dry THF under nitrogen. TMS acetylene (351.7 mg, 3.581 mmol, 1.2 eq., 0.50 mL) was added to the mixture and Et₃N (905.9 mg, 8.953 mmol, 3 eq., 1.2 mL) was added last. After 10½ hours of stirring at room temperature additional TMS acetylene was added to the reaction and was stirred for further 12 hours. The reaction was then diluted with EtOAc, filtered through Celite and washed again with EtOAc. The reaction was concentrated under reduced pressure. Purification of the resulting crude product by flash column chromatography (Hx/EA gradient, 0→100% EA) gave MB-56 (893 mg, 2.925 mmol, 98%) as a light brown solid. R_{f}=0.46 [Hx:EA, 7:3]. ¹H NMR (400 MHz, Chloroform-d) δ=7.90-7.85 (m, 1H), 6.78-6.74 (m, 1H), 3.89 (s, 3H), 3.83 (s, 3H), 0.27 (s, 9H) ppm. ¹³C NMR (100 MHz, CDCl₃) δ=169.30, 166.19, 150.94, 136.11, 134.95, 118.09, 112.97, 108.63, 102.37, 99.71, 52.88, 52.32, 0.12 ppm. HRMS (ESI): calcd. for C₁₅H₁₉NNaO₄S⁺: 328.0976 m/z [M+Na]⁺; found: 328.1032 m/z [M+Na]⁺. LCMS (ESI): tᵣₑₜ=4.42 min. 306 m/z [M+H]⁺.

### Dimethyl 4-amino-5-ethynylphthalate (MB-59)

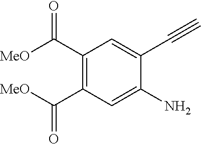 [SMILES: C#Cc1cc(C(=O)OC)c(C(=O)OC)cc1N]

MB-56 (873.1 mg, 2.859 mmol, 1 eq.) and K2CO₃ (790.2 mg, 5.718 mmol, 2 eq.) were suspended in dry MeOH (15 mL) under nitrogen. After 4 hours of stirring the reaction was diluted with water (100 mL), extracted with CH₂Cl₂ (3×75 mL) and EtOAc (3×75 mL) and washed twice with brine (2×100 mL). The organic phase was dried over Na₂SO₄ and concentrated under reduced pressure. MB-59 (580 mg, 2.487 mmol, 87%) was obtained as a light orange crystalline solid. R_{f}=0.26 [Hx:EA, 7:3]. ¹H NMR (400 MHz, DMSO-d₆) δ=7.66 (s, 1H), 6.77 (s, 1H), 6.41 (s, 2H), 4.52 (s, 1H), 3.76 (s, 3H), 3.72 (s, 3H) ppm. ¹³C NMR (100 MHz, DMSO-d₆) δ=168.57, 165.20, 153.15, 135.99, 134.48, 114.52, 112.33, 105.28, 87.05, 78.92, 52.41, 51.94 ppm. HRMS (ESI): calcd. for C₁₂H₁₁NNaO₄⁺: 256.0580 m/z [M+Na]⁺; found: 256.0633 m/z [M+Na]⁺. LCMS (ESI): tᵣₑₜ=3.02 min. 234 m/z [M+H]⁺.

### (Z)-8,9-bis(methoxycarbonyl)-11,12-dihydrodibenzo[c,g][1,2]diazocine-3-carboxylic Acid (MB-62)

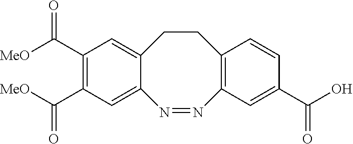 [SMILES: COC(=O)c1cc2c(cc1C(=O)OC)/N=N\c1cc(C(=O)O)ccc1CC2]

MB-50 (17.0 mg, 0.040 mmol, 1 eq.) was dissolved in CH₂Cl₂: TFA (1:1, 2 mL:2 mL) and stirred for 3 hours at room temperature. The reaction was then concentrated under reduced pressure, triturated twice with MeOH and dried on high vacuum overnight. MB-61 (14.2 mg, 0.039 mmol, 96%) was obtained as a yellow/brownish solid. R_{f}=0.29 [Hx:EA, 1:5]. ¹H NMR (400 MHz, Chloroform-d) δ=7.78 (d, J=7.9 Hz, 1H), 7.60 (s, 1H), 7.37 (s, 1H), 7.24 (s, 1H), 7.12 (d, J=8.0 Hz, 1H), 3.84 (d, J=5.4 Hz, 6H), 3.13-2.98 (m, 2H), 2.89 (t, J=9.8 Hz, 2H) ppm. ¹³C NMR (100 MHz, CDCl₃) δ 170.45, 167.43, 166.80, 156.65, 155.05, 133.51, 131.29, 131.08, 130.87 (2C), 130.32, 129.53, 128.70, 120.94, 120.19, 77.48, 52.93, 52.91, 31.65, 31.47 ppm. HRMS (APCI): calcd. for C₁₉H₁₇N₂O₆⁺: 369.1081 m/z [M+H]⁺; found: 369.1087 m/z [M+H]⁺. LCMS (ESI): tᵣₑₜ=3.28 min. 369 m/z [M+H]⁺.

### Dimethyl (Z)-8-((6-((tert-butoxycarbonyl)amino)hexyl)carbamoyl)-11,12-dihydrodibenzo[c,g][1,2]diazocine-2,3-dicarboxylate (MB-68)

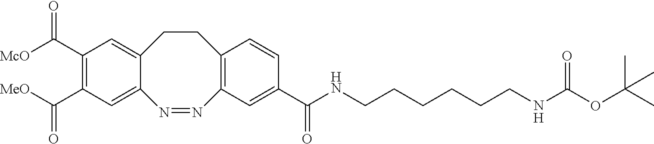

MB-62 (14.1 mg, 0.038 mmol, 1 eq.) and HATU (18.6 mg, 0.057 mmol, 1.5 eq.) were dissolved in dry DMF (1 mL) under nitrogen. After 5 minutes of stirring N-Boc-1,4-diaminohexane (33.1 mg, 0.153 mmol, 4 eq.) and iPr₂Net (19.8 mg, 0.153 mmol, 4 eq., 27 μL) were added to the mixture and stirred for further 13 hours at room temperature. The reaction was diluted with EtOAc (20 mL), separated against 5% LiCl (20 mL), extracted with EtOAc (3×20 mL) and washed with 10% LiCl (2×20 mL) and brine (2×20 mL). The combined organic phase was dried over Na₂SO₄ and concentrated under reduced pressure. Purification of the resulting crude product by flash column chromatography (Hx/Ea gradient, 0-100% Ea) gave MB-68 (15.0 mg, 0.026 mmol, 69%) as a yellow oil. R_{f}=0.16 [Hx:EtOAc, 1:1]. ¹H NMR (400 MHz, Chloroform-d) δ=7.48 (d, J=7.7 Hz, 1H), 7.35 (d, J=3.7 Hz, 2H), 7.22 (s, 1H), 7.05 (d, J=7.9 Hz, 1H), 6.39 (s, 1H), 4.56 (s, 1H), 3.85 (s, 3H), 3.84 (s, 3H), 3.43-3.32 (m, 2H), 3.15-3.07 (m, 2H), 3.06-2.95 (m, 2H), 2.92-2.78 (m, 2H), 1.58 (p, J=6.1, 5.6 Hz, 2H), 1.49-1.30 (m, 6H), 1.42 (s, 9H) ppm. ¹³C NMR (100 MHz, CDCl₃) δ=167.49, 166.87, 166.13, 156.73, 156.30, 155.10, 134.05, 131.54, 131.01, 130.80, 130.69, 130.57, 130.22, 126.20, 120.28, 118.11, 79.24, 52.91, 52.89, 40.11, 39.72, 31.71, 31.26, 29.83, 29.44, 28.54, 26.14, 25.89 ppm. HRMS (APCI): calcd. for C₃₀H₃₉N₄O₇⁺: 567.2813 m/z [M+H]⁺; found: 567.2801 m/z [M+H]⁺. LCMS (ESI): tᵣₑₜ=4.20 min. 589 m/z [M+Na]⁺.

### Ethyl 5-amino-1,3-dioxoisoindoline-2-carboxylate (MB-84)

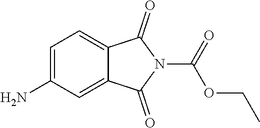 [SMILES: CCOC(=O)N1C(=O)c2ccc(N)cc2C1=O]

MB-84 was prepared similar to the previously described method: 4-Aminophthalimide (3.0 g, 18.50 mmol, 1 eq.) was suspended in MeCN (45 mL) and Et₃N (6.4 mL, 46.25 mmol, 2.5 eq.) at 0° C. While stirring a solution of ethyl chloroformate (2.7 mL, 27.75 mmol, 1.5 eq.) in MeCN (6 mL) was added dropwise over 60 minutes. After additional two hours of stirring at 0° C. the reaction was warmed to room temperature and stirred for further 1 hour. The reaction was concentrated under reduced pressure and a solution of 1 M hydrochloric acid (1.8 mL) in distilled water (44 mL) was added and the mixture is again cooled to 0° C. and stirred for further 1 hour at this temperature. The solid product was filtered and washed twice with a solvent mixture (MeCN: water; 1:4, 2×20 mL) and dried three days on high vacuum. MB-84 (4070 mg, 17.38 mmol, 94%) was obtained as a mustard yellow solid. R_{f}=0.03 [Hx:EA, 8:2]. ¹H NMR (400 MHz, DMSO-d₆) δ=7.58 (d, J=8.3 Hz, 1H), 6.95 (s, 1H), 6.90 (d, J=8.5 Hz, 1H), 6.70 (s, 2H), 4.31 (q, J=7.1 Hz, 2H), 1.29 (t, J=7.1 Hz, 3H) ppm. ¹³C NMR (100 MHz, DMSO) δ=164.26, 163.26, 155.95, 148.34, 133.69, 126.05, 118.59, 115.64, 106.78, 62.89, 13.97 ppm. LCMS (ESI): tᵣₑₜ=2.72 min. 235 m/z [M+H]⁺.

### Ethyl 5-amino-1,3-dioxo-6-((trimethylsilyl)ethynyl)isoindoline-2-carboxylate (MB-78)

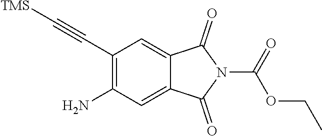 [SMILES: CCOC(=O)N1C(=O)c2cc(N)c(C#C[Si](C)(C)C)cc2C1=O]

MB-73 (360 mg, 1.0 mmol, 1 eq.), CuI (95.2 mg, 0.5 mmol, 0.5 eq.) and PdCl₂(PPH₃)₂ (350.8 mg, 0.5 mmol, 0.5 eq.) were suspended in dry THF (12 mL) under nitrogen. TMS acetylene (117.8 mg, 1.2 mmol, 1.2 eq., 0.17 mL) was added to the mixture and Et₃N (303.5 mg, 2.99 mmol, 3 eq., 0.73 mL) was added last. After 6 hours of stirring at room temperature the reaction was then diluted with EtOAc, filtered through Celite and washed again with EtOAc. The reaction was concentrated under reduced pressure. Purification of the resulting crude product by flash column chromatography (Hx/EA gradient, 0→100% EA) gave MB-78 (33.1 mg, 0.10 mmol, 10%) as a light yellow solid. R_{f}=0.06 [Hx:EA, 8:2]. ¹H NMR (400 MHz, Chloroform-d) δ=7.85 (s, 1H), 7.13 (s, 1H), 4.46 (q, J=7.1 Hz, 2H), 1.43 (t, J=7.1 Hz, 3H) ppm. ¹³C NMR (100 MHz, CDCl₃) δ=163.22, 162.84, 154.65, 148.89, 132.65, 125.56, 119.97, 118.47, 109.15, 103.67, 95.77, 63.92, 14.30, 0.00 ppm. HRMS (ESI): calcd. for C₁₆H₁₉N₂O₄S⁺: 331.1109 m/z [M+H]⁺; found: 331.1108 m/z [M+H]⁺. LCMS (ESI): tᵣₑₜ=4.13 min. 331 m/z [M+H]⁺.

### 5-amino-6-iodoisoindoline-1,3-dione (MB-87)

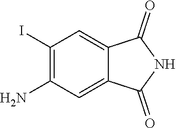 [SMILES: Nc1cc2c(cc1I)C(=O)NC2=O]

4-Aminophthalimide (2.0 g, 12.33 mmol, 1 eq.) and NIS (5.55 g, 24.67 mmol, 2 eq.) were dissolved in dry DMF (40 mL) and stirred at 45° C. overnight. While stirring, water (60 mL) was added and the precipitate was filtered and washed with water (80 mL), Na₂SO₄ (6 g in 60 mL) and again with water (80 mL). MB-87 (2.860 g, 9.93 mmol, 81%) was obtained as a light brown solid. R_{f}=0.10 [Hx:EA, 8:2]. ¹H NMR (400 MHz, DMSO-d₆) δ=10.89 (s, 1H), 7.92 (s, 1H), 7.03 (s, 1H), 6.45 (s, 2H) ppm. ¹³C NMR (100 MHz, DMSO) δ=169.11, 167.98, 153.96, 134.91, 133.89, 119.88, 106.64, 86.48 ppm. LCMS (ESI): tᵣₑₜ=2.59 min. 289 m/z [M+H]⁺. The analytical data matched those previously described.

### Methyl 4-amino-3-((trimethylsilyl)ethynyl)benzoate (MB-89)

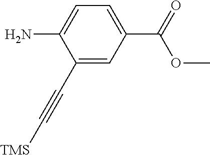 [SMILES: COC(=O)c1ccc(N)c(C#C[Si](C)(C)C)c1]

Methyl 4-amino 3-iodobenzoate (2000 mg, 7.22 mmol, 1 eq.), CuI (68.7 mg, 0.361 mmol, 0.05 eq.) and Pd(PPh₃)₂Cl₂ (253.3 mg, 0.361 mmol, 0.05 eq.) were dissolved in dry THF (19 mL) under nitrogen at room temperature. TMS acetylene (850.8 mg, 8.66 mmol, 1.2 eq., 1.2 mL) was added to the mixture and Et₃N (2192.4 mg, 21.656 mmol, 3 eq., 3.0 mL) was added last. After 7 hours of stirring the reaction was diluted with EtOAc (25 mL), filtered through Celite and washed again with EtOAc (50 mL). The reaction was concentrated under reduced pressure. Purification of the resulting crude product by flash column chromatography (Hx/EA gradient, 0→100% EA) gave MB-89 (1715 mg, 6.93 mmol, 96%) as brown crystals. R_{f}=0.40 [Hx:EA, 8:2]. ¹H NMR (400 MHz, Chloroform-d) δ=8.01 (s, 1H), 7.79 (dd, J=8.5, 2.3 Hz, 1H), 6.66 (d, J=8.6 Hz, 1H), 4.65 (s, 2H), 3.85 (s, 3H), 0.26 (s, 9H) ppm. ¹³C NMR (100 MHz, DMSO) δ 166.67, 151.96, 134.75, 131.74, 119.41, 113.28, 107.19, 100.65, 100.60, 51.86, 0.20 ppm. LCMS (ESI): tᵣₑₜ=4.46 min. 248 m/z [M+H]⁺.

### Ethyl 5-amino-6-iodo-1,3-dioxoisoindoline-2-carboxylate (MB-90)

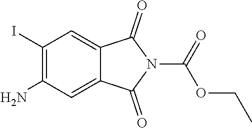 [SMILES: CCOC(=O)N1C(=O)c2cc(N)c(I)cc2C1=O]

MB-87 (2800 mg, 9.72 mmol, 1 eq.) was dissolved in MeCN (24 mL) and Et₃N (3.4 mL) at 0° C. A solution of ethyl chloroformate (1.4 mL) in MeCN (4 mL) was added dropwise over 1 hour at 0° C. After addition of ethyl chloroformate the mixture was stirred for further 2 hours at 0° C. The reaction was then warmed up to room temperature and stirred for 1 hour. The reaction was concentrated under reduced pressure. A solution of 1 M hydrochloric acid (0.95 mL) in distilled water (23 mL) was added and the mixture is again cooled to 0° C. and stirred for 1 hour at this temperature. The solid product was filtered and washed twice with a solvent mixture (MeCN/water; 1:4; 2×10 mL) and dried for 2 days on high vacuum. MB-90 (3320 mg, 9.219 mmol, 95%) was obtained as a light brown solid. R_{f}=0.08 [Hx:EA, 8:2]. ¹H NMR (400 MHz, DMSO-d₆) δ=8.08 (s, 1H), 7.10 (s, 1H), 6.72 (s, 2H), 4.32 (q, J=7.2 Hz, 2H), 1.29 (t, J=7.2 Hz, 3H) ppm. ¹³C NMR (100 MHz, DMSO-d₆): δ=163.83, 162.03, 155.06, 148.20, 135.23, 133.07, 117.59, 106.64, 88.63, 63.02, 13.96 ppm. HRMS (APCI): calcd. for C₁₁H₁₀IN₂O₄⁺: 360.9678 m/z [M+H]⁺; found: 360.9680 m/z [M+H]⁺. LCMS (ESI): tᵣₑₜ=2.76 min. 393 m/z [M+H+MeOH]⁺.

### Methyl 4-amino-3-ethynylbenzoate (MB-91)

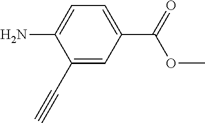

MB-89 (1680 mg, 6.791 mmol, 1 eq.) and K2CO₃ (1877 mg, 13.58 mmol, 2 eq.) were suspended in dry MeOH (35 mL) under nitrogen. After 2 hours of stirring the reaction was diluted with EtOAc (50 mL), separated against H₂O (60 mL), extracted with EtOAc (2×50 mL) and washed twice with brine (2×80 mL). The organic phase was dried over Na₂SO₄ and concentrated under reduced pressure. Purification of the resulting crude product by flash column chromatography (Hx/EA gradient, 0→100% EA) gave MB-91 (1020 mg, 5.82 mmol, 86%) as a brown crystalline solid. R_{f}=0.48 [Hx:EA, 2:1]. ¹H NMR (¹H NMR (400 MHz, Chloroform-d) δ=8.04 (s, 1H), 7.82 (dd, J=8.6, 2.2 Hz, 1H), 6.67 (dd, J=8.5, 1.9 Hz, 1H), 4.67 (s, 2H), 3.86 (d, J=1.8 Hz, 3H), 3.40 (d, J=1.8 Hz, 1H) ppm. ¹³C NMR (100 MHz, CDCl₃) δ=166.59, 152.25, 135.08, 132.00, 119.48, 113.41, 105.96, 83.11, 79.64, 51.91 ppm. LCMS (ESI): tᵣₑₜ=3.03 min. 176 m/z [M+H]⁺. The analytical data matched those previously described.

### Ethyl 5-amino-6-((2-amino-4-(methoxycarbonyl)phenyl)ethynyl)-1,3-dioxoisoindoline-2-carboxylate (MB-92)

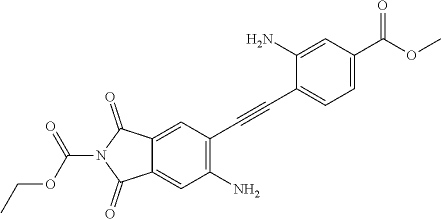 [SMILES: CCOC(=O)N1C(=O)c2cc(N)c(C#Cc3ccc(C(=O)OC)cc3N)cc2C1=O]

MB-90 (946.0 mg, 5.4 mmol, 1.2 eq.), MB-91 (1620.5 mg, 4.5 mmol, 1 eq.), CuI (42.9 mg, 0.225 mmol, 0.05 eq.) and PdCl₂(PPh₃)₂ (157.9 mg, 0.225 mmol, 0.05 eq.) were suspended in dry THF (24 mL) under nitrogen. Et₃N (1366.1 mg, 13.5 mmol, 3 eq., 1.9 mL) was added last. After 5 hours of stirring the reaction was diluted with EtOAc (25 mL) and the precipitate was collected by filtration and washed with EtOAc:MeOH (9:1, 9 mL:1 mL). MB-92 was obtained (446.2 mg, 1.085 mmol, 80%) as a yellow solid. R_{f}=0.47 [CH₂Cl₂: MeOH, 19:1]. ¹H NMR (4 00 MHz, DMSO-d₆) δ=8.18 (s, 2H), 7.67 (d, J=8.8 Hz, 1H), 7.14 (s, 1H), 7.00 (s, 2H), 6.75 (d, J=8.7 Hz, 1H), 6.52 (s, 2H), 4.34 (q, J=7.0 Hz, 2H), 3.78 (s, 3H), 1.31 (t, J=7.0 Hz, 3H) ppm. ¹³C NMR (100 MHz, DMSO-d₆) δ=165.78, 163.78, 162.95, 154.93, 153.55, 148.28, 134.94, 132.17, 131.37, 129.38, 116.14, 116.07, 113.25, 111.25, 107.31, 104.46, 94.07, 89.70, 62.99, 51.41, 13.99 ppm. HRMS (ESI): calcd. for C₂₁H₁₈N₃O₆⁺: 408.1190 m/z [M+H]⁺; found: 408.1203 m/z [M+H]⁺. LCMS (ESI): tᵣₑₜ=3.85 min. 408 m/z [M+H]⁺.

### Ethyl 5-amino-6-(2-amino-4-(methoxycarbonyl)phenethyl)-1,3-dioxoisoindoline-2-carboxylate (MB-93)

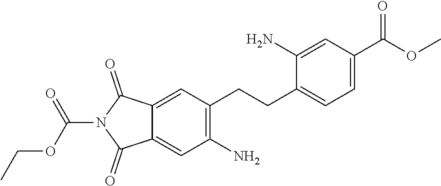 [SMILES: CCOC(=O)N1C(=O)c2cc(N)c(CCc3ccc(C(=O)OC)cc3N)cc2C1=O]

MB-92 (550.0 mg, 1.350 mmol, 1 eq.) and Pd/C (143.7 mg, 0.135 mmol, 0.1 eq.) were dissolved in dry MeOH (26 ml) under nitrogen. The flask was then charged with hydrogen gas and the reaction mixture was stirred for 5 h. The reaction was filtered by using Celite, washed with hot MeOH/EtOAc (1:1; 1 L) and was concentrated under reduced pressure. MB-93 (446.2 mg, 1.085 mmol, 80%) was obtained as a faint yellow solid. R_{f}=0.44 [CH₂Cl₂:MeOH, 19:1]. ¹H NMR (400 MHz, DMSO-d₆) δ=7.68-7.59 (m, 2H), 7.55 (dd, J=8.4, 2.1 Hz, 1H), 7.06 (s, 1H), 6.64 (d, J=8.4 Hz, 1H), 6.56 (s, 2H), 5.88 (s, 2H), 4.32 (q, J=7.1 Hz, 2H), 3.73 (s, 3H), 2.81-2.70 (m, 4H), 1.30 (t, J=7.0 Hz, 3H) ppm. ¹³C NMR (100 MHz, DMSO-d₆) δ=166.51, 164.31, 163.55, 153.75, 151.30, 148.40, 131.24, 130.95, 130.82, 128.87, 125.30, 123.18, 116.15, 116.08, 113.38, 107.32, 62.87, 51.12, 29.11, 28.44, 13.99 ppm. HRMS (ESI): calcd. for C₂₁H₂₁N₃NaO₆⁺: 434.1503 m/z [M+Na]⁺; found: 434.1304 m/z [M+Na]⁺. LCMS (ESI): tᵣₑₜ=3.49 min. 412 m/z [M+H]⁺.

### tert-butyl (Z)-(9-bromo-11,12-dihydrodibenzo[c,g][1,2]diazocin-2-yl)carbamate (BM-1)

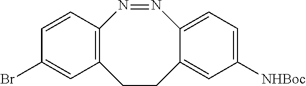 [SMILES: CC(C)(C)OC(=O)Nc1ccc2c(c1)CCc1cc(Br)ccc1/N=N\2]

To a solution of (Z)-2-bromo-9-iodo-11,12-dihydrodibenzo[c,g][1,2]diazocine (100 mg, 0.24 mmol, 1.0 equiv) in 1,4-dioxane (4.8 mL) was added t-Bu carbamate (31 mg, 0.27 mmol, 1.1 equiv), Cs₂CO₃ (118 mg, 0.36 mmol, 1.5 equiv) and XantPhos G3 precatalyst (5.8 mg, 6 umol, 0.025 equiv). The reaction was evacuated and backfilled with nitrogen and heated to 100° C. overnight. The reaction solution was directly loaded onto celite and purified by column chromatography over SiO₂ using a 98:2 Hex/EtOAc to 9:1 Hex/EtOAc gradient to elute the product BM-1 (88 mg, 0.22 mmol, 90% yield) as a yellow amorphous solid. ¹H NMR (400 MHz, Chloroform-d) δ 7.26-7.22 (m, 1H), 7.18 (s, 1H), 7.14 (d, J=2.0 Hz, 1H), 7.05 (dd, J=8.6, 2.2 Hz, 1H), 6.79 (d, J=8.5 Hz, 1H), 6.70 (d, J=8.3 Hz, 1H), 6.48 (s, 1H), 2.83 (s, 4H), 1.51-1.46 (m, 9H). ¹³C NMR (101 MHz, CDCl₃) δ 154.4, 152.6, 150.7, 137.7, 132.4, 130.6, 129.9, 128.7, 125.6, 120.8, 120.4, 120.3, 119.0, 116.7, 80.9, 31.9, 31.5, 28.4.

### Methyl (Z)-9-((tert-butoxycarbonyl)amino)-11,12-dihydrodibenzo[c,g][1,2]diazocine-2-carboxylate (BM-2)

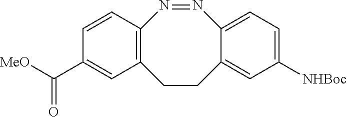 [SMILES: COC(=O)c1ccc2c(c1)CCc1cc(NC(=O)OC(C)(C)C)ccc1/N=N\2]

BM-1 (71 mg, 0.18 mmol, 1.0 equiv) and XantPhos G3 precatalyst (6.7 mg, 7.1 umol, 0.04 equiv) were dissolved in triethylamine (2.4 mL). The reaction solution was sparged with carbon monoxide and fitted with a balloon of carbon monoxide. To this was added MeOH (214 uL, 170 mg, 5.3 mmol, 30 equiv) and the reaction solution heated to 70° C. and allowed to react overnight. The headspace of the reaction was sparged with nitrogen for 5 minutes, ventilating into the hood. The reaction was diluted with EtOAc, washed with ice-cold 1M HCl. The aqueous layer was back extracted three times with EtOAc, the organics were combined, washed with ice-cold 1M HCl, saturated sodium bicarbonate, and brine. The organic layer was dried over sodium sulfate and concentrated. The residue was purified by column chromatography over SiO₂ using a 95:5 Hex/EtOAc to 7:3 Hex/EtOAc gradient to afford the desired product BM-2 (47 mg, 0.12 mmol, 70% yield) as a yellow amorphous solid. ¹H NMR (400 MHz, Chloroform-d) δ 7.78 (dd, J=8.2, 1.7 Hz, 1H), 7.68 (d, J=1.7 Hz, 1H), 7.17-7.09 (m, 1H), 7.04 (dd, J=8.5, 2.2 Hz, 1H), 6.84 (d, J=8.2 Hz, 1H), 6.78 (d, J=8.4 Hz, 1H), 6.52 (s, 1H), 3.84 (s, 2H), 3.07-2.64 (m, 5H), 1.45 (s, 9H). ¹³C NMR (101 MHz, CDCl₃) δ 166.4, 159.2, 152.6, 150.8, 137.7, 131.2, 128.9, 128.8, 128.6, 128.3, 120.1, 119.0, 118.8, 116.7, 80.9, 52.2, 32.1, 31.3, 28.4.

### Methyl (S,Z)-9-(2-(4-(4-chlorophenyl)-2,3,9-trimethyl-6H-thieno[3,2-f][1,2,4]triazolo[4,3-a][1,4]diazepin-6-yl)acetamido)-11,12-dihydrodibenzo[c,g][1,2]diazocine-2-carboxylate (BM-3)

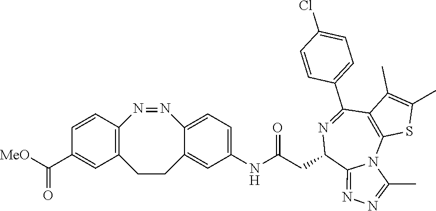 [SMILES: COC(=O)c1ccc2c(c1)CCc1cc(NC(=O)C[C@@H]3N=C(c4ccc(Cl)cc4)c4c(sc(C)c4C)-n4c(C)nnc43)ccc1/N=N\2]

BM-2 (4.4 mg, 11.5 umol, 1.0 equiv) was dissolved in formic acid (1.0 mL) at room temperature. After 30 min, the reaction was complete and concentrated in vacuo and azeotroped three times with EtOAc to afford a yellow film. This film was dissolved in dry DMF (250 ul). In a separate vial containing (+)-JQ1 free acid (5.1 mg, 12.7 umol, 1.1 equiv) in DMF (563 ul) was added HATU (5.3 mg, 13.8 umol, 1.2 equiv) and DIPEA (10 ul, 7.5 mg, 57.7 umol, 5.0 equiv). This solution was allowed to stir at room temperature for 15 minutes. This solution was transferred to a solution of deprotected BM-2 in DMF (250 ul) and allowed to stir overnight at room temperature. After this period, the reaction was diluted with EtOAc, washed twice with 10% LiCl solution in water, saturated sodium bicarbonate, and brine. The organics were dried over sodium sulfate and concentrated in vacuo. The yellow residue was purified by column chromatography over SiO₂ using a 7:3 Hex/EtOAc to 100% EtOAc gradient to afford product BM-3 (6.8 mg, 10.2 umol, 87% yield) as a yellow film. ¹H NMR (400 MHz, Chloroform-d) δ 8.95 (s, 1H), 7.79 (dd, J=8.1, 1.8 Hz, 1H), 7.69 (s, 1H), 7.39 (d, J=8.3 Hz, 2H), 7.32 (d, J=8.4 Hz, 2H), 6.83 (dd, J=19.5, 8.3 Hz, 2H), 5.12 (s, 1H), 4.57 (dd, J=8.9, 4.9 Hz, 1H), 3.85 (s, 3H), 3.73 (dd, J=14.1, 8.8 Hz, 1H), 3.42 (dd, J=14.1, 4.8 Hz, 1H), 2.97 (d, J=9.9 Hz, 2H), 2.80 (dt, J=19.8, 9.0 Hz, 2H), 2.67 (s, 3H), 2.40 (s, 3H).

### (Z)-9-(2-((S)-4-(4-chlorophenyl)-2,3,9-trimethyl-6H-thieno[3,2-f][1,2,4]triazolo[4,3-a][1,4]diazepin-6-yl)acetamido)-N—((S)-1-((2 S,4R)-4-hydroxy-2-((4-(4-methylthiazol-5-yl)benzyl)carbamoyl)pyrrolidin-1-yl)-3,3-dimethyl-1-oxobutan-2-yl)-11,12-dihydrodibenzo[c,g][1,2]diazocine-2-carboxamide (BM-389)

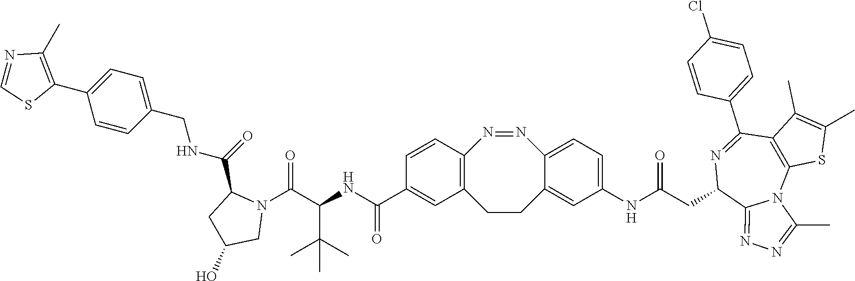 [SMILES: Cc1ncsc1-c1ccc(CNC(=O)[C@@H]2C[C@@H](O)CN2C(=O)[C@@H](NC(=O)c2ccc3c(c2)CCc2cc(NC(=O)C[C@@H]4N=C(c5ccc(Cl)cc5)c5c(sc(C)c5C)-n5c(C)nnc54)ccc2/N=N\3)C(C)(C)C)cc1]

To a solution of BM-3 (6.5 mg, 9.8 umol, 1.0 equiv) in 1:1 THF/MeOH (500 ul) was added LiOH (1M soln. in H₂O, 250 ul, 0.25 mmol, 25.5 equiv) in a dropwise fashion. The reaction was allowed to stir at r.t. for 2 hours upon which the reaction was diluted with water. The aqueous layer was extracted 3 times with EtOAc. To the aqueous layer was added ice, followed by an equal volume of ice-cold 2M HCl solution in water. This aqueous fraction was extracted 3 times with EtOAc, which was combined with the previous organic fraction, which was successively washed with sodium bicarbonate and brine. The organics were dried over sodium sulfate and concentrated to an amorphous yellow solid (5.5 mg, 8.5 umol, 86% yield) that was used directly in the next step. To a solution of BM-3 free acid (5.0 mg, 7.7 umol, 1.0 equiv) in DMF (0.5 mL) was added HATU (4.4 mg, 11.5 umol, 1.5 equiv) and DIPEA (7 ul, 5.2 mg, 40.2 umol, 5.2 equiv). This mixture was allowed to stir at room temperature for 15 minutes, followed by the dropwise addition of the VHL ligand (3.6 mg, 8.5 umol, 1.1 equiv) in DMF (500 ul). The reaction was allowed to stir overnight, upon which it was diluted with EtOAc, washed with two portions of 10% LiCl solution in water, sat. NaHCO₃, and brine. The organics were dried over sodium sulfate and concentrated. The residue was purified by column chromatography over SiO₂ using 100% DCM to 95:5 DCM/MeOH gradient to afford the desired product BM-389 (5.7 mg, 5.4 umol, 70% yield) as a yellow film. ¹H NMR (600 MHz, Chloroform-d) δ 9.17 (d, J=45.6 Hz, 1H), 8.71 (s, 1H), 7.82-7.50 (m, 2H), 7.48-7.38 (m, 3H), 7.35 (q, J=5.3, 4.3 Hz, 6H), 6.87-6.66 (m, 2H), 4.81-4.60 (m, 3H), 4.52 (d, J=22.7 Hz, 2H), 4.45-4.25 (m, 0H), 4.08 (d, J=11.4 Hz, 1H), 3.69 (t, J=12.2 Hz, 2H), 3.45 (d, J=14.9 Hz, 1H), 3.16 (qd, J=7.3, 4.9 Hz, 1H), 3.04-2.87 (m, 2H), 2.86-2.68 (m, 1H), 2.65 (s, 3H), 2.51 (s, 3H), 2.42 (s, 3H), 2.03 (q, J=39.6, 31.3 Hz, 11H), 1.74-1.63 (m, 3H), 1.28 (d, J=2.1 Hz, 2H), 1.01 (d, J=11.7 Hz, 9H). LCMS (ESI): =4.81 min 531.8 m/z [M+2E1]²⁺.

### Methyl (Z)-9-((tert-butoxycarbonyl)amino)-11,12-dihydrodibenzo[c,g][1,2]diazocine-3-carboxylate (BM-4)

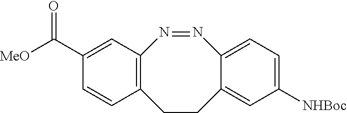 [SMILES: COC(=O)c1ccc2c(c1)/N=N\c1ccc(NC(=O)OC(C)(C)C)cc1CC2]

To a solution of Methyl (Z)-9-iodo-11,12-dihydrodibenzo[c,g][1,2]diazocine-3-carboxylate (55 mg, 0.14 mmol, 1.0 equiv) in 1,4-dioxane (1.4 mL) was added t-Bu carbamate (33 mg, 0.28 mmol, 2.0 equiv), Cs₂CO₃ (114 mg, 0.35 mmol, 2.5 equiv) and XantPhos G3 precatalyst (3.3 mg, 3.5 umol, 0.025 equiv). The reaction was evacuated and backfilled with nitrogen and heated to 100° C. overnight. The reaction solution was filtered over a pad of celite, until no yellow color persisted and loaded onto celite and purified by column chromatography over SiO₂ using a 95:5 Hex/EtOAc to 7:3 Hex/EtOAc gradient to elute the product BM-4 (53 mg, 0.14 mmol, 99% yield) as a yellow amorphous solid. ¹H NMR (400 MHz, Chloroform-d) δ 7.66 (dd, J=8.0, 1.8 Hz, 1H), 7.45 (d, J=1.8 Hz, 1H), 7.16 (s, 1H), 7.08-6.97 (m, 2H), 6.77 (d, J=8.5 Hz, 1H), 6.73 (s, 1H), 4.55 (s, 1H), 3.83 (s, 3H), 2.94 (d, J=10.0 Hz, 2H), 2.86-2.66 (m, 2H), 1.43 (s, 9H). ¹³C NMR (101 MHz, CDCl₃) δ 166.2, 155.3, 152.6, 150.5, 137.6, 133.7, 129.8, 128.8, 128.4, 128.1, 120.2, 120.1, 118.8, 116.7, 80.7, 60.4, 52.2, 31.8, 31.5, 28.2, 21.0, 14.2.

### tert-butyl ((Z)-8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamoyl)-11,12-dihydrodibenzo[c,g][1,2]diazocin-2-yl)carbamate (BM-5)

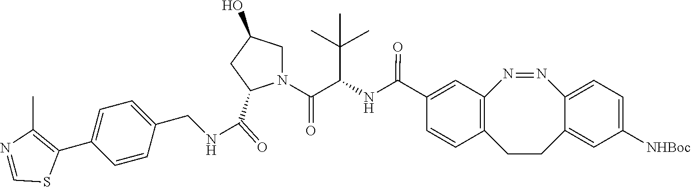 [SMILES: Cc1ncsc1-c1ccc(CNC(=O)[C@@H]2C[C@@H](O)CN2C(=O)[C@@H](NC(=O)c2ccc3c(c2)/N=N\c2ccc(NC(=O)OC(C)(C)C)cc2CC3)C(C)(C)C)cc1]

To a solution of BM-4 (25 mg, 66 umol, 1.0 equiv) in 1:1 THF/MeOH (0.5 mL) was added LiOH (1M soln. in H₂O, 250 ul, 0.25 mmol, 3.8 equiv) in a dropwise fashion. The reaction was allowed to stir at r.t. for 3.75 hours upon which the reaction was diluted with water. The aqueous layer was extracted 3 times with EtOAc. To the aqueous layer was added ice, followed by an equal volume of ice-cold 2M HCl solution in water. This aqueous fraction was extracted 3 times with EtOAc, which was combined with the previous organic fraction, which was successively washed with water and brine. The organics were dried over sodium sulfate and concentrated to an amorphous yellow solid (24 mg, 82 umol, 99% yield) which was used without further purification. To a solution of deprotected BM-4 (9.4 mg, 25.5 umol, 1.1 equiv) in DMF (0.5 mL) was added HATU (13.2 mg, 34.8 umol, 1.5 equiv) and DIPEA (16 ul, 12 mg, 92 umol, 4.0 equiv). This mixture was allowed to stir at room temperature for 15 minutes followed by the dropwise addition of the VHL ligand (10.0 mg, 23.2 umol, 1.0 equiv) in DMF (500 ul). The reaction was allowed to stir overnight, upon which the reaction was diluted with EtOAc, washed with two portions of 10% LiCl solution in water, sat. NaHCO₃, and brine. The organics were dried over sodium sulfate and concentrated. The residue was purified by column chromatography over SiO₂ using 100% DCM to 95:5 DCM/MeOH gradient to afford the desired product BM-5 (13 mg, 16.7 umol, 72% yield) as a yellow film. ¹H NMR (500 MHz, Chloroform-d) δ 8.68 (s, 1H), 7.46-7.29 (m, 7H), 7.24-7.17 (m, 2H), 6.92 (d, J=30.9 Hz, 2H), 6.83-6.68 (m, 1H), 6.65-6.44 (m, 2H), 4.78-4.50 (m, 2H), 4.48 (s, 1H), 4.31 (dd, J=15.1, 5.1 Hz, 1H), 4.06 (s, 1H), 3.62 (dd, J=11.4, 3.7 Hz, 1H), 2.93 (s, 2H), 2.76 (s, 2H), 2.50 (s, 4H), 2.45 (s, 0H), 2.05 (dd, J=34.7, 18.0 Hz, 3H), 1.45 (s, 10H), 0.95 (d, J=13.7 Hz, 9H). ¹³C NMR (101 MHz, CDCl₃) δ 171.6, 171.2, 170.8, 166.5, 166.0, 155.3, 152.5, 150.5, 150.3, 148.4, 138.1, 137.6, 132.7, 132.0, 131.7, 130.9, 130.1, 129.5, 128.8, 128.1, 125.8, 124.6, 120.4, 118.8, 117.9, 116.7, 80.8, 70.3, 58.7, 57.7, 56.9, 43.3, 36.2, 35.8, 35.5, 31.6, 29.7, 29.4, 28.3, 26.5, 22.7, 16.0, 14.1.

### (Z)-9-(2-((S)-4-(4-chlorophenyl)-2,3,9-trimethyl-6H-thieno[3,2-f][1,2,4]triazolo[4,3-a][1,4]diazepin-6-yl)acetamido)-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-11,12-dihydrodibenzo[c,g][1,2]diazocine-3-carboxamide (BM-399)

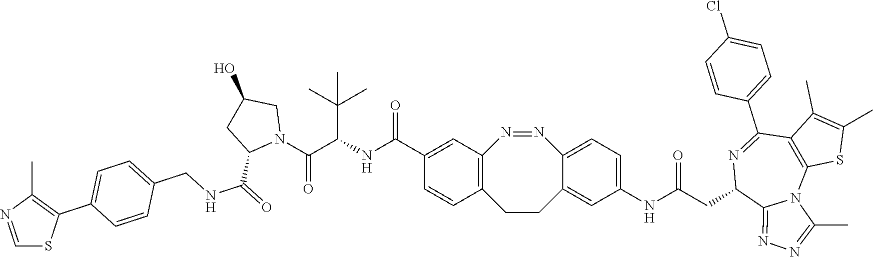 [SMILES: Cc1ncsc1-c1ccc(CNC(=O)[C@@H]2C[C@@H](O)CN2C(=O)[C@@H](NC(=O)c2ccc3c(c2)/N=N\c2ccc(NC(=O)C[C@@H]4N=C(c5ccc(Cl)cc5)c5c(sc(C)c5C)-n5c(C)nnc54)cc2CC3)C(C)(C)C)cc1]

BM-5 (7.7 mg, 7.7 umol, 1.0 equiv) was dissolved in formic acid (1.0 mL) at room temperature. After 1.5 hours, the reaction was complete and concentrated in vacuo and azeotroped three times with EtOAc to afford a yellow film. This film was dissolved in dry DMF (250 ul). In a separate vial containing (+)-JQ1 free acid (3.7 mg, 9.2 umol, 1.2 equiv) in DMF (259 ul) was added HATU (3.5 mg, 9.2 umol, 1.2 equiv) and DIPEA (7 ul, 5.0 mg, 38.5 umol, 5.0 equiv). This solution was allowed to stir at room temperature for 15 minutes. This solution was transferred to a solution of deprotected BM-5 in DMF (250 ul) and allowed to stir overnight at room temperature. After this period, the reaction was diluted with EtOAc, washed twice with 10% LiCl solution in water, saturated sodium bicarbonate, and brine. The organics were dried over sodium sulfate and concentrated in vacuo. The yellow residue was purified by column chromatography over SiO₂ using a 100% DCM to 9:1 DCM/MeOH gradient to afford product BM-399 (1.3 mg, 1.2 umol, 16% yield) as a yellow film. (NOTE: ¹H NMR contains broadened peaks due to the presence of multiple rotamers). ¹H NMR (400 MHz, Chloroform-d) δ 9.13 (d, J=26.2 Hz, 1H), 8.77 (s, 1H), 7.74 (dd, J=5.7, 3.4 Hz, 0H), 7.64-7.51 (m, 1H), 7.45-7.32 (m, 6H), 7.00 (d, J=18.0 Hz, 0H), 6.94-6.56 (m, 2H), 4.92-4.51 (m, 5H), 4.46-4.25 (m, 1H), 4.24-3.98 (m, 1H), 3.71 (dd, J=25.7, 11.4 Hz, 2H), 3.48 (d, J=15.0 Hz, 1H), 2.87 (dd, J=77.5, 12.0 Hz, 4H), 2.69 (s, 2H), 2.54 (s, 3H), 2.43 (s, 3H), 1.70 (s, 3H), 1.33-1.24 (m, 7H). LCMS (ESI): tᵣₑₛ=4.28 min 1062.3 m/z [M+H]⁺.

### 3-(4-((4-Hydroxy-3,5-diisopropylphenyl)diazenyl)-1-oxoisoindolin-2-yl)piperidine-2,6-dione (KK-32)

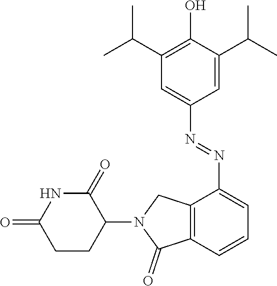 [SMILES: CC(C)c1cc(/N=N/c2cccc3c2CN(C2CCC(=O)NC2=O)C3=O)cc(C(C)C)c1O]

Lenalidomide (519 mg, 2.000 mmol, 1.0 eq.) was dissolved in 1 M HCl (20 mL) and concentrated aq. HBF₄ (2 mL) was added to the mixture. After completely dissolving of the starting material, 2 M NaNO₂ (1.1 mL) was added to the solution at 0° C. After stirring for 1 h the solution was added dropwise into a mixture of propofol (357 mg, 2.000 mmol, 1.0 eq.) in H₂O (50 mL), MeOH (20 mL), NaHCO₃ (4.15 g, 49.37 mmol, 24.7 eq.) and Na₂CO₃ (5.18 g, 49.37 mmol, 24.7 eq.) and stirred for an additional 1 h at 0° C. The reaction was extracted with EtOAc (7×50 mL) and washed once with brine (1×50 mL). The organic phase was dried over Na₂SO₄ and concentrated under reduced pressure. Purification of the resulting crude product by flash column chromatography (CH₂Cl₂/MeOH gradient, 0→10% MeOH) gave KK-32 (519.0 mg, 1.157 mmol, 58%) as a yellow solid. R_{f}=0.30 [CH₂Cl₂: MeOH, 19:1]. ¹H NMR (400 MHz, DMSO) δ=11.01 (s, 1H), 8.13 (d, J=7.7 Hz, 1H), 7.84 (d, J=7.4 Hz, 1H), 7.74 (t, J=7.0 Hz, 1H), 7.66 (s, 2H), 5.17-5.06 (m, 1H), 4.75 (dd, J=42.7, 18.9 Hz, 2H), 3.37 (dd, J=13.7, 6.9 Hz, 2H), 2.98-2.85 (m, 1H), 2.69-2.52 (m, 2H), 2.12-1.95 (m, 1H), 1.24 (s, 6H), 1.23 (s, 6H) ppm. ¹³C NMR (400 MHz, CDCl₃) δ=170.77, 169.66, 168.79, 166.69, 153.80, 147.29, 147.15, 134.54, 134.43, 133.25, 129.25, 128.30, 125.22, 119.18, 82.47, 82.38, 52.58, 48.19, 42.07, 31.97, 28.13, 28.02, 27.44, 22.66, 22.63 ppm. HRMS (ESI): calcd. For C₂₅H₂₇N₄O₄⁺:449.2183 m/z [M+H]⁺. Found: 449.2188 m/z [M+H]⁺. LCMS (ESI): tᵣₑₜ=4.05 min. 49 m/z [M+H]⁺.

### tert-Butyl-2-(4-((2-(2,6-dioxopiperidin-3-yl)-1-oxoisoindolin-4-yl)diazenyl)-2,6-diisopropylphenoxy)acetate (KK-84)

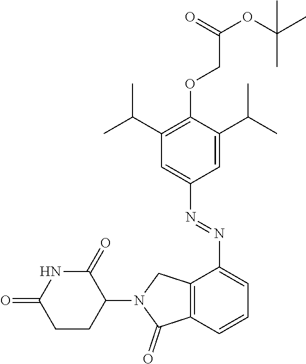 [SMILES: CC(C)c1cc(/N=N/c2cccc3c2CN(C2CCC(=O)NC2=O)C3=O)cc(C(C)C)c1OCC(=O)OC(C)(C)C]

To tert-Butyl bromoacetate (52.4 mg, 269.0 μmol, 1.2 eq., 40 μL) was added dry DMF (10 mL), KK-32 (101 mg, 224 μmol, 1.0 eq.) and iPr₂NEt (43.8 mg, 336.0 μmol, 1.5 eq., 59.0 μL) at room temperature. After stirring for 3 h, the mixture was diluted with EtOAc (20 mL), separated against NaHCO₃ (30 mL), extracted with EtOAc (3×30 mL) and washed with 10% aq. LiCl (3×30 mL) and brine (2×30 mL). The reaction was concentrated under reduced pressure. Purification of the resulting crude product by flash column chromatography (Hexane/EtOAc gradient, 20→100% EtOAc) gave KK-84 (83.0 mg, 147.5 mmol, 66%) as a yellow solid. R_{f}=0.29 [EtOAc:Hexane, 1:1]. ¹H NMR (400 MHz, CDCl₃) δ=8.25 (d, J=7.7 Hz, 1H), 7.95 (d, J=7.5 Hz, 1H), 7.67 (t, J=7.7 Hz, 1H), 7.53 (d, J=6.3 Hz, 1H), 6.55 (s, 1H), 5.35-5.26 (m, 1H), 4.76 (q, J=18.8 Hz, 1H), 4.71 (s, 1H), 3.99 (s, 3H), 3.94 (s, 3H), 3.10-3.00 (m, 1H), 3.00-2.85 (m, 1H), 2.46 (qd, J=13.2, 4.4 Hz, 1H), 2.30-2.19 (m, 1H), 1.50 (s, 9H) ppm. ¹³C NMR (400 MHz, CDCl₃) δ=172.91, 171.43, 168.78, 167.14, 154.45, 153.02, 147.95, 144.38, 135.47, 133.48, 133.07, 130.76, 129.25, 124.56, 99.14, 98.81, 82.91, 66.64, 56.98, 56.46, 53.97, 51.78, 49.41, 30.50, 28.09, 28.07, 23.88 ppm. HRMS (ESI): calcd. For C₃₂H₄₂N₄O₇Na⁺: 617.2951 m/z [M+MeOH+Na]⁺. Found: 617.2942 m/z [M+MeOH+Na]⁺. LCMS (ESI): tᵣₑₜ=4.32 min. 563 m/z [M+H]⁺.

### tert-Butyl-2-(4-((2-(2,6-dioxopiperidin-3-yl)-1-oxoisoindolin-4-yl)diazenyl)-2,5-dimethoxyphenoxy)acetate (KK-85)

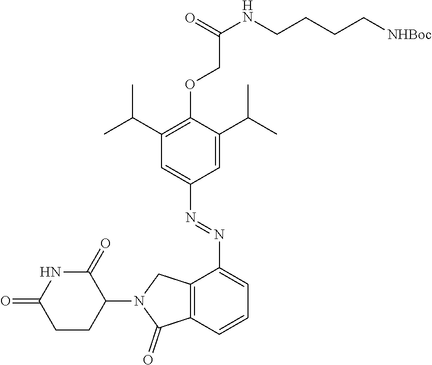 [SMILES: CC(C)c1cc(/N=N/c2cccc3c2CN(C2CCC(=O)NC2=O)C3=O)cc(C(C)C)c1OCC(=O)NCCCCNC(=O)OC(C)(C)C]

KK-84 (84.2 mg, 149.6 μmol, 1.0 eq.) was dissolved in a CH₂Cl₂:TFA mixture (1:1; 4 mL). After 6 h the reaction was concentrated under reduced pressure. The mixture was triturated with MeOH and then dried under high vacuum for 24 h. The crude solid was dissolved in dry DMF (5 mL) at room temperature and HATU (86.1 mg, 226.5 μmol, 2.0 eq) was added to the mixture. After 5 min of stirring N-Boc-1,4-diaminobutane (56.3 mg, 299.3 μmol, 2.0 eq., 57 μL) and iPr₂NEt (598.6 μmol, 4.0 eq., 104 μL) were added to the mixture and stirred for additional 12 h at room temperature. The reaction was diluted with EtOAc (20 mL), separated against a H2O:10% aq. LiCl mixture (1:1, 20 mL), extracted with EtOAc (2×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH radiant, 0-20% MeOH) gave KK-85 (60.0 mg, 88.70 μmol, 59%) as a yellow solid. R_{f}=0.33 [CH₂Cl₂:MeOH, 19:1]. ¹H NMR (400 MHz, DMSO) δ=10.99 (d, J=17.3 Hz, 1H), 8.22 (t, J=8.3 Hz, 2H), 7.90 (t, J=9.3 Hz, 1H), 7.79 (t, J=7.6 Hz, 1H), 7.73 (s, 2H), 6.81 (s, 1H), 5.13 (dt, J=12.7, 6.4 Hz, 1H), dd, (J=42.6, 19.0 Hz, 2H), 4.25 (s, 2H), 3.22-3.20 (m, 3H), 2.99-2.87 (m, 3H), 2.70-2.53 (m, 3H), 2.15-1.97 (m, 1H), 1.55-1.39 (m, 4H), 1.38 (s, 9H), 1.27 (s, 6H), 1.25 (s, 6H) ppm. ¹³C NMR (400 MHz, DMSO) δ=172.95, 171.03, 167.20, 167.01, 155.61, 149.53, 146.58, 142.88, 134.62, 133.82, 129.60, 128.34, 125.34, 118.89, 77.36, 73.25, 52.02, 48.37, 39.52, 38.12, 31.31, 28.30, 27.02, 26.54, 26.33, 23.78, 22.29 ppm. HRMS (ESI): calcd. For C₃₆H₄₈N₆O₇⁺:677.3663 m/z [M+H]⁺. Found: 677.3644 m/z [M+H]⁺. LCMS (ESI): tᵣₑₜ=4.31 min. 621 m/z [M+H-t-Butyl]⁺.

### N-(4-Aminobutyl)-2-(4-((2-(2,6-dioxopiperidin-3-yl)-1-oxoisoindolin-4-yl)diazenyl)-2,6-diisopropylphenoxy)acetamide (KK-34)

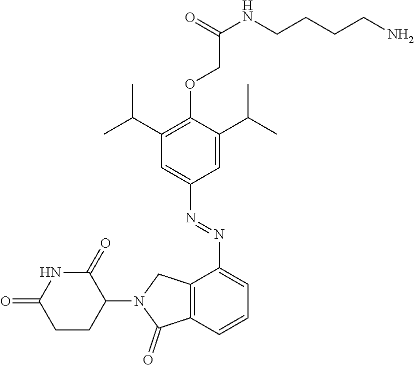 [SMILES: CC(C)c1cc(/N=N/c2cccc3c2CN(C2CCC(=O)NC2=O)C3=O)cc(C(C)C)c1OCC(=O)NCCCCN]

KK-85 (67.2 mg, 99.30 μmol, 1 eq.) was dissolved in a CH₂Cl₂:TFA mixture (1:1; 4 mL). After 6 h the reaction was concentrated under reduced pressure. The mixture was triturated with MeOH and then dried under high vacuum for 48 h. KK-34 (56.0 mg, 97.10 μmol, 98%) was obtained as orange solid with traces of residual TFA. R_{f}=0.13 [CH₂Cl₂₊₁% NEt₃:MeOH, 5:1]. ¹H NMR (400 MHz, DMSO) δ=11.02 (s, 1H), 8.32 (t, J=5.8 Hz, 1H), 8.21 (d, J=7.3 Hz, 1H), 7.92 (d, J=7.2 Hz, 1H), 7.79 (dd, J=13.7, 6.1 Hz, 1H), 7.74 (s, 2H), 7.70 (s, 2H) 5.13 (dd, J=12.7, 6.4 Hz, 1H), 4.77 (dd, J=42.8, 19.0 Hz, 4H), 4.26 (s, 2H), 3.22 (d, J=5.9 Hz, 2H), 3.00-2.77 (m, 3H), 2.69-2.52 (m, 2H), 2.12-2.01 (m, 1H), 1.63-1.47 (m, 4H), 1.28 (s, 6H), 1.26 (s, 6H) ppm. ¹³C NMR (400 MHz, DMSO) δ=172.94, 171.02, 167.18, 155.49, 149.56, 146.56, 142.85, 134.61, 133.82, 129.59, 128.30, 125.35, 118.89, 73.21, 51.98, 48.31, 38.58, 37.69, 31.29, 26.30, 26.14, 24.46, 23.79, 23.76, 23.42, 22.28 ppm. HRMS (ESI): calcd. For C₃₁H₄₀N₆O₅⁺: 577.3133 m/z [M+H]⁺. Found: 577.3132 m/z [M+H]⁺. LCMS (ESI): tᵣₑₜ=2.46 min. 577 m/z [M+H]⁺.

### 2-((S)-4-(4-chlorophenyl)-2,3,9-trimethyl-6H-thieno[3,2-f][1,2,4]triazolo[4,3-a][1,4]diazepin-6-yl)-N-(4-(2-(4-((2-(2,6-dioxopiperidin-3-yl)-1-oxoisoindolin-4-yl)diazenyl)-2,6-diisopropylphenoxy)acetamido)butyl)acetamide (KR-86)

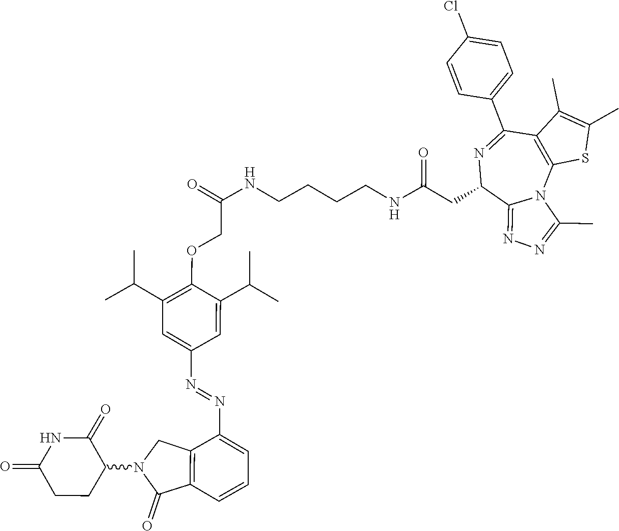 [SMILES: Cc1sc2c(c1C)C(c1ccc(Cl)cc1)=N[C@@H](CC(=O)NCCCCNC(=O)COc1c(C(C)C)cc(/N=N/c3cccc4c3CN(C3CCC(=O)NC3=O)C4=O)cc1C(C)C)c1nnc(C)n1-2]

Into a round bottom flask with dry (+)-JQ acid (9.50 mg, 23.70 μmol, 1 eq.) were added KK-34 (27.3 mg, 47.40 μmol, 2 eq.) and HATU (13.5 mg, 35.50 μmol, 1.5 eq.) under nitrogen atmosphere. The solids were dissolved in dry DMF (1 mL). After addition of i-Pr₂NEt (165.9 μmol, 7.0 eq., 29 μL) the reaction was stirred for 24 h at room temperature. The mixture was then diluted with EtOAc (20 mL), separated against 10% LiCl (30 mL), extracted with EtOAc (2×20 mL), washed twice with 10% LiCl (2×20 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) gave KR-86 (14.0 mg, 14.6 μmol, 62%) as an orange solid. R_{f}=0.09 [CH₂Cl₂:MeOH, 19:1]. ¹H NMR (400 MHz, CDCl₃) δ=8.35 (t, J=11.1 Hz, 1H), 8.19 (t, J=11.7 Hz, 1H), 7.99 (d, J=7.4 Hz, 1H), 7.70 (t, J=7.7 Hz, 1H), 7.66 (s, 2H), 7.42 (d, J=8.2 Hz, 2H), 7.34 (d, J=8.4 Hz, 2H), 7.03 (dd, J=12.1, 6.2 Hz, 1H), 7.00 (d, J=16.3 Hz, 1H), 5.24 (dd, J=13.2, 4.8 Hz, 1H), 4.79 (dd, J=45.2, 18.0 Hz, 2H), 4.68 (t, J=6.8 Hz, 1H), 4.32 (s, 2H), 3.59 (dt, J=25.3, 12.7 Hz, 1H), 3.49-3.41 (m, 2H), 3.39-3.30 (m, 2H), 3.24 (dt, J=13.7, 6.8 Hz, 2H), 2.92-2.84 (m, 2H), 2.68 (s, 3H), 2.50-2.45 (m, 1H), 2.40 (s, 3H), 2.31-2.16 (m, 1H), 1.73-1.68 (m, 4H), 1.67 (s, 3H), 1.32 (s, 6H), 1.30 (s, 6H) ppm. ¹³C NMR (400 MHz, CDCl₃) δ=171.17, 170.32, 169.55, 168.56, 168.18, 164.55, 155.30, 154.77, 150.31, 150.11, 147.03, 142.83, 137.34, 135.91, 134.09, 133.27, 132.05, 131.53, 131.15, 130.48, 130.06, 129.90, 129.55, 129.42, 128.84, 125.97, 119.29, 72.98, 54.32, 52.05, 48.21, 39.18, 38.96, 38.77, 31.63, 29.70, 27.07, 26.83, 23.94, 23.41, 14.41, 13.15, 11.76 ppm. LCMS (ESI): tᵣₑₜ=4.44 min. 959 m/z [M+H]⁺.

### 3-(4-((4-Hydroxy-3-methylnaphthalen-1-yl)diazenyl)-1-oxoisoindolin-2-yl)piperidine-2,6-dione (KK-35)

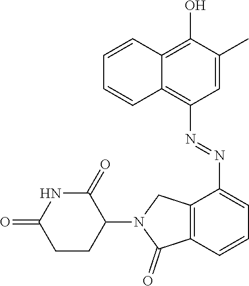 [SMILES: Cc1cc(/N=N/c2cccc3c2CN(C2CCC(=O)NC2=O)C3=O)c2ccccc2c1O]

Lenalidomide (200.0 mg, 0.771 mmol, 1.0 eq.) was dissolved in 1 M HCl (20 mL) and concentrated aq. HBF₄ (2 mL) was added to the mixture. After completely dissolving of the starting material, 2 M NaNO₂ solution (58.5 mg, 0.849 mmol, 1.1 eq., 424 μL) was added to the mixture at 0° C. After stirring for 1 h the solution was added dropwise into a mixture of 2-Methylnaphthalen-1-ol (122.0 mg, 0.771 mmol, 1.0 eq.) in H₂O (20 mL), MeOH (20 mL), NaHCO₃ (1.60 g, 19.05 mmol, 24.7 eq.) and Na₂CO₃ (2.02 g, 19.05 mmol, 24.7 eq.) and stirred for an additional 1 h at 0° C. Filtration and washing of the solid residue with EtOAc (3×15 mL) gave KK-35 (151 mg, 0.353 mmol, 46%) as an orange solid. R_{f}=0.23 [CH₂Cl₂:MeOH, 19:1]. ¹H NMR (400 MHz, DMSO) δ=11.06 (s, 1H), 8.25 (s, 2H), 8.10 (s, 1H), 7.74 (t, J=7.4 Hz, 1H), 7.63 (s, J=12.2 Hz, 1H), 7.56 (t, J=7.4 Hz, 2H), 7.42 (s, 1H), 5.19 (dd, J=13.1, 5.1 Hz, 1H), 4.82 (dd, J=45.0, 17.8 Hz, 2H), 3.03-2.89 (m, 1H), 2.76-2.61 (m, 1H), 2.45-2.30 (m, 1H), 2.26-2.06 (m, 1H) ppm. HRMS (ESI): calcd. For C₂₅H₂₄N₄O₅Na⁺: 483.1644 m/z [M+MeOH+Na]⁺. Found: 483.1756 m/z [M+MeOH+Na]⁺. LCMS (ESI): tᵣₑₜ=2.97 min. 451 m/z [M+Na]⁺.

### tert-Butyl-2-((4-((2-(2,6-dioxopiperidin-3-yl)-1-oxoisoindolin-4-yl)diazenyl)-2-methylnaphthalen-1-yl)oxy)acetate (KK-86)

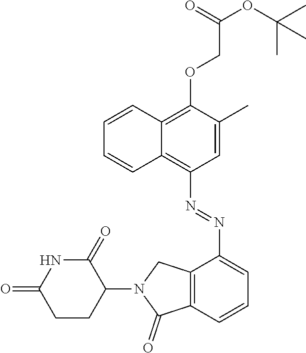 [SMILES: Cc1cc(/N=N/c2cccc3c2CN(C2CCC(=O)NC2=O)C3=O)c2ccccc2c1OCC(=O)OC(C)(C)C]

To tert-Butyl bromoacetate (100.7 mg, 516.2 μmol, 1.2 eq., 76 μL) was added dry DMF (5 mL), KK-35 (194.0 mg, 430.2 μmol, 1.0 eq.) and iPr₂NEt (860.3 mmol, 2.0 eq., 150 μL) at room temperature. After stirring for 2.5 h, the mixture was diluted with EtOAc (20 mL), separated against NaHCO₃ (20 mL), extracted with EtOAc (3×20 mL) and washed with 10% LiCl (3×20 mL) and brine (2×20 mL). The reaction was concentrated under reduced pressure. Purification of the resulting crude product by flash column chromatography (Hexane/EtOAc gradient, 20→100% EtOAc) gave KK-86 (99.0 mg, 182.5 mmol, 42%) as an orange solid. R_{f}=0.40 [EtOAc:Hexane 4:1]. ¹H NMR (400 MHz, DMSO) δ=11.04 (s, 1H), 8.77 (d, J=8.2 Hz, 1H), 8.34 (d, J=3.6 Hz, 2H), 7.96 (d, J=7.4 Hz, 1H), 7.88-7.79 (m, 2H), 7.77-7.68 (m, 2H), 5.20 (dd, J=13.1, 5.0 Hz, 1H), 4.90 (dd, J=55.8, 18.5 Hz, 2H), 4.68 (s, 2H), 2.99-2.88 (m, 1H), 2.73-2.58 (m, 2H), 2.52 (s, 3H), 2.13 (dd, J=19.0, 12.9 Hz, 1H), 1.50 (s, 9H) ppm. ¹³C NMR (400 MHz, DMSO) δ=173.39, 171.50, 168.27, 167.70, 156.29, 147.46, 144.06, 135.53, 134.28, 131.08, 130.18, 128.53, 128.23, 127.83, 127.41, 126.81, 125.99, 123.43, 122.79, 116.31, 82.08, 71.36, 52.44, 48.88, 31.73, 28.22, 22.92, 16.84 ppm. LCMS (ESI): tᵣₑₜ=3.73 min. 543 m/z [M+H]⁺.

### tert-Butyl-(4-(2-((4-((2-(2,6-dioxopiperidin-3-yl)-1-oxoisoindolin-4-yl)diazenyl)-2-methylnaphthalen-1-yl)oxy)acetamido)butyl)carbamate (KK-87)

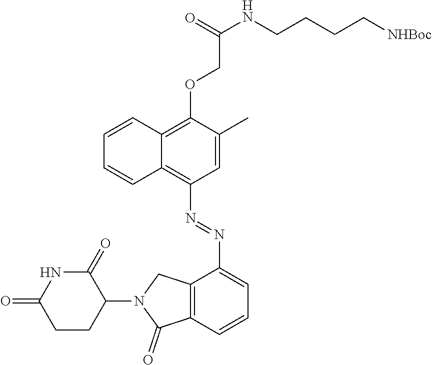 [SMILES: Cc1cc(/N=N/c2cccc3c2CN(C2CCC(=O)NC2=O)C3=O)c2ccccc2c1OCC(=O)NCCCCNC(=O)OC(C)(C)C]

KK-86 (92.5 mg, 170.6 mmol, 1 eq.) was dissolved in a CH₂Cl₂:TFA mixture (1:1; 2 mL). After 4 h the reaction was concentrated under reduced pressure. The mixture was triturated with MeOH and then dried under high vacuum for 48 h. HATU (97.3 mg, 255.9 μmol, 1.5 eq.) was added and the mixture was dissolved in dry DMF (5 mL) at room temperature. After 5 min of stirring N-Boc-1,4-diaminobutane (64.2 mg, 341.2 μmol, 2.0 eq., 65 μL) and iPr₂NEt (604.0 μmol, 4.0 eq., 119 μL) were added to the mixture and stirred for additional 12 h at room temperature. The reaction was diluted with EtOAc (20 mL), separated against H₂O/10% LiCl (1:1, 10 mL:10 mL), extracted with EtOAc (2×20 mL) and washed with 10% LiCl (2×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave KK-87 (105.1 mg, 160.0 μmol, 94%) as a yellow solid. R_{f}=0.23 [CH₂Cl₂:MeOH, 19:1]. ¹H NMR (400 MHz, DMSO) δ=8.77 (dd, J=5.6, 3.9 Hz, 1H), 8.42 (t, J=5.8 Hz, 1H), 8.33 (d, J=7.7 Hz, 1H), 8.26-8.18 (m, 1H), 7.90 (d, J=7.4 Hz, 1H), 7.81 (dd, J=14.5, 6.9 Hz, 2H), 7.71 (p, J=6.0 Hz, 2H), 7.65 (s, 1H), 7.24 (s, 1H), 5.00 (dd, J=53.3, 18.9 Hz, 2H), 4.80 (dd, J=10.2, 5.0 Hz, 1H), 4.45 (s, 2H), 3.20 (q, J=6.4 Hz, 2H), 2.94 (q, J=6.4 Hz, 2H), 2.50 (d, J=4.3 Hz, 3H), 2.38-2.29 (m, 2H), 2.29-2.09 (m, 2H), 1.54-1.39 (m, 4H), 1.36 (s, 9H) ppm. ¹³C NMR (400 MHz, CDCl₃) δ=173.89-173.65 (m), 172.99 (s), 171.59 (s), 168.69 (s), 168.31 (s), 156.20 (s), 154.34 (s), 147.54 (s), 144.74 (s), 131.68 (s), 129.55 (s), 128.83 (s), 128.21 (s), 127.38 (s), 126.71 (s), 125.91 (s), 123.71 (s), 115.53 (s), 72.03 (s), 54.20 (s), 51.98 (s), 48.71 (s), 38.96 (s), 30.57 (s), 29.82 (s), 28.55 (s), 27.74 (s), 27.13 (s), 24.41 (s), 16.42 (s) ppm. LCMS (ESI): tᵣₑₜ=3.60 min. 689 m/z [M+MeOH+H]⁺.

### N-(4-Aminobutyl)-2-((4-((2-(2,6-dioxopiperidin-3-yl)-1-oxoisoindolin-4-yl)diazenyl)-2-methylnaphthalen-1-yl)oxy)acetamide (KK-37)

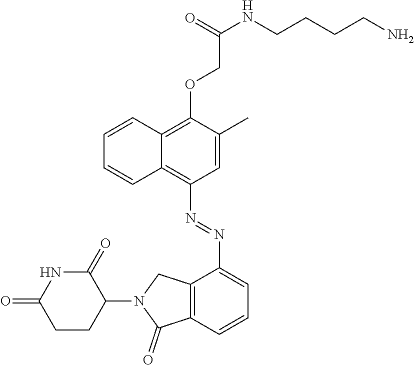 [SMILES: Cc1cc(/N=N/c2cccc3c2CN(C2CCC(=O)NC2=O)C3=O)c2ccccc2c1OCC(=O)NCCCCN]

KK-86 (22.0 mg, 33.50 μmol, 1.0 eq.) was dissolved in a CH₂Cl₂:TFA mixture (1:1; 2 mL). After 3 h the reaction was concentrated under reduced pressure. The mixture was triturated with MeOH and then dried under high vacuum for 48 h. KK-37 (18.6 mg, 33.50 μmol, >99%) was obtained as red solid with traces of residual TFA. R_{f}=0.13 [CH₂Cl₂:MeOH, 2:1]. ¹H NMR (400 MHz, DMSO) δ=8.80 (d, J=9.4 Hz, 1H), 8.52 (t, J=5.8 Hz, 1H), 8.35 (d, J=7.7 Hz, 1H), 8.27-8.20 (m, 1H), 7.93 (d, J=7.4 Hz, 1H), 7.86 (s, 1H), 7.83 (t, J=7.7 Hz, 1H), 7.76-7.71 (m, 2H), 7.69 (s, 1H), 7.26 (s, 1H), 5.02 (dd, J=55.5, 18.9 Hz, 2H), 4.83 (dd, J=10.1, 5.1 Hz, 1H), 4.48 (s, 2H), 3.27 (d, J=5.6 Hz, 3H), 2.85 (d, J=5.8 Hz, 2H), 2.54 (s, 3H), 2.40-2.10 (m, 6H), 1.23 (s, 1H) ppm. ¹³C NMR (400 MHz, MeOD) δ=174.60, 170.99, 169.95, 155.99, 148.41, 145.27, 134.79, 134.42, 132.80, 131.89, 130.36, 129.39, 128.21, 127.94, 126.21, 124.56, 122.53, 115.97, 72.86, 55.51, 52.27, 50.67, 49.00, 40.38, 39.36, 31.44, 27.56, 26.52, 25.93, 16.42 ppm. LCMS (ESI): tᵣₑₜ=2.98 min. 589 m/z [M+MeOH+H]⁺.

### 2-((S)-4-(4-Chlorophenyl)-2,3,9-trimethyl-6H-thieno[3,2-f][1,2,4]triazolo[4,3-a][1,4]diazepin-6-yl)-N-(4-(2-((4-((2-(2,6-dioxopiperidin-3-yl)-1-oxoisoindolin-4-yl)diazenyl)-2-methylnaphthalen-1-yl)oxy)acetamido)butyl)acetamide (KR-93)

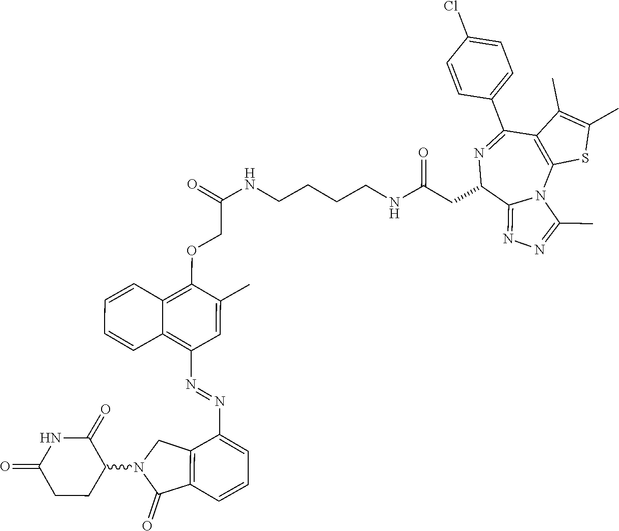 [SMILES: Cc1cc(/N=N/c2cccc3c2CN(C2CCC(=O)NC2=O)C3=O)c2ccccc2c1OCC(=O)NCCCCNC(=O)C[C@@H]1N=C(c2ccc(Cl)cc2)c2c(sc(C)c2C)-n2c(C)nnc21]

(+)-JQ1 free acid (8.5 mg, 21.20 μmol, 1.0 eq.), KK-37 (23.6 mg, 42.40 mmol, 2.0 eq.) and HATU (12.1 mg, 31.80 μmol, 1.5 eq.) were dissolved in dry DMF (1 mL). After addition of i-Pr₂NEt (148.4 μmol, 7.0 eq., 26 μL) the reaction was stirred for 24 h at room temperature. The mixture was then diluted with EtOAc (20 mL), separated against 5% LiCl (10 mL), extracted with EtOAc (2×10 mL) and washed twice with 10% LiCl (2×10 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) and HPLC gave KR-93 (8.4 mg, 8.90 μmol, 42%) as a red solid. R_{f}=0.16 [CH₂Cl₂:MeOH, 19:1]. ¹H NMR (400 MHz, CDCl₃) δ=8.71 (dd, J=8.3, 3.5 Hz, 1H), 8.20 (dd, J=7.7, 3.4 Hz, 1H), 7.96 (dd, J=16.0, 8.5 Hz, 2H), 7.71-7.57 (m, 4H), 7.39 (d, J=8.4 Hz, 2H), 7.31 (d, J=8.5 Hz, 3H), 7.09-7.00 (m, 1H), 6.71 (d, J=26.2 Hz, 1H), 4.98 (dd, J=14.6, 4.4 Hz, 2H), 4.91 (dd, J=41.5, 11.5 Hz, 2H), 4.65 (t, J=6.6 Hz, 1H), 4.47 (s, J=2.4 Hz, 2H), 3.62 (d, J=7.2 Hz, 3H), 3.54-3.46 (m, 2H), 3.44-3.32 (m, 3H), 2.61 (s, 3H), 2.46 (s, J=2.7 Hz, 3H), 2.37 (s, 3H), 2.34-2.20 (m, 2H), 1.80-1.67 (m, 4H), 1.65 (s, 3H) ppm. ¹³C NMR (400 MHz, CDCl₃) δ=172.67, 171.38, 170.33, 168.39, 168.02, 163.86, 155.38, 154.16, 149.75, 147.18, 147.17, 144.32, 136.70, 136.25, 134.48, 133.35, 131.84, 131.34, 130.80, 130.27, 129.66, 129.19, 128.54, 127.91, 127.03, 126.41, 125.51, 123.28, 121.14, 115.18, 77.16, 54.30, 53.90, 51.65, 48.42, 39.15, 38.94, 38.67, 30.26, 29.49, 26.81, 24.19, 24.12, 16.14, 14.17, 12.88, 11.55 ppm. HRMS (ESI): calcd. For C₅₀H₅₂ClN₁₀O₇S⁺: 971.2821 m/z [M+MeOH+H]⁺. Found: 971.2825 m/z [M+MeOH+H]⁺ LCMS (ESI): tᵣₑₜ=3.32 min. 971 m/z [M+MeOH+H]⁺.

### 3-(4-((4-Hydroxy-2,6-dimethylphenyl)diazenyl)-1-oxoisoindolin-2-yl)piperidine-2,6-dione (KK-38)

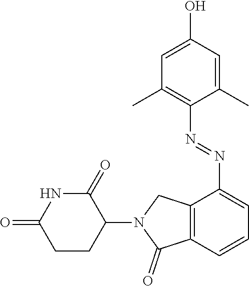 [SMILES: Cc1cc(O)cc(C)c1/N=N/c1cccc2c1CN(C1CCC(=O)NC1=O)C2=O]

Lenalidomide (518.5 mg, 2.000 mmol, 1.0 eq.) was dissolved in 1 M HCl (30 mL). Concentrated aqueous HBF₄ (2 mL) was added to the mixture. After completely dissolving of the starting material, 2 M NaNO₂ solution (1.1 mL) was added to the mixture at 0° C. After stirring for 1 h the solution was added dropwise into a mixture of 3,5-Dimethylphenol (244.3 mg, 2.000 mmol, 1.0 eq.) in H₂O (50 mL), MeOH (20 mL), NaHCO₃ (4.15 g, 49.37 mmol, 24.7 eq.) and Na₂CO₃ (5.18 g, 49.37 mmol, 24.7 eq.) and stirred for an additional 1 h at 0° C. The reaction was extracted with EtOAc (7×50 mL) and washed once with brine (1×50 mL). The organic phase was dried over Na₂SO₄ and concentrated under reduced pressure. Purification of the resulting crude product by flash column chromatography (CH₂Cl₂/MeOH gradient, 0→10% MeOH) gave KK-38 (706.0 mg, 1.799 mmol, 90%) as a red solid. R_{f}=0.30 [CH₂Cl₂:MeOH, 19:1]. ¹H NMR (400 MHz, DMSO) δ=11.00 (s, 1H), 10.11 (s, 1H), 8.08 (d, J=7.8 Hz, 1H), 7.85 (d, J=7.4 Hz, 1H), 7.75 (t, J=7.6 Hz, 1H), 6.63 (s, 2H), 5.15 (dd, J=13.2, 5.0 Hz, 1H), 4.67 (dd, J=50.8, 18.5 Hz, 2H), 2.97-2.87 (m, 1H), 2.63 (t, J=15.5 Hz, 1H), 2.48-2.44 (m, 1H), 2.46 (s, 6H), 2.10-2.01 (m, 1H) ppm. ¹³C NMR (400 MHz, DMSO) δ=173.14, 171.26, 167.63, 159.47, 147.46, 142.96, 136.36, 134.48, 133.83, 129.72, 126.69, 124.50, 116.57, 39.52, 31.48, 22.66, 21.18 ppm. LCMS (ESI): tᵣₑₜ=3.30 min. 393 m/z [M+H]⁺.

### tert-Butyl-2-(4-((2-(2,6-dioxopiperidin-3-yl)-1-oxoisoindolin-4-yl)diazenyl)-3,5-dimethylphenoxy)acetate (KK-87)

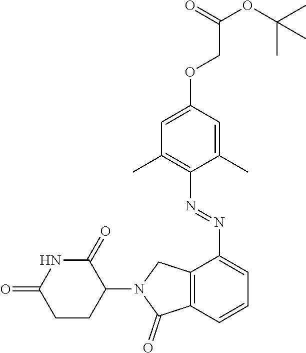 [SMILES: Cc1cc(OCC(=O)OC(C)(C)C)cc(C)c1/N=N/c1cccc2c1CN(C1CCC(=O)NC1=O)C2=O]

To tert-Butyl bromoacetate (150.0 mg, 769.1 μmol, 1.2 eq., 114 μL) was added dry DMF (6 mL), KK-38 (250.0 mg, 637.1 μmol, 1.0 eq.) and iPr₂NEt (1.274 mmol, 2.0 eq., 222 μL) at room temperature. After stirring for 2.5 h, the mixture was diluted with EtOAc (100 mL), separated against NaHCO₃ (50 mL), extracted with EtOAc (3×50 mL) and washed with 10% LiCl (3×50 mL) and brine (2×50 mL). The mixture was concentrated under reduced pressure. Purification of the resulting crude product by flash column chromatography (Hexane/EtOAc gradient, 20→100% EtOAc) gave KK-87 (184.0 mg, 363.2 mmol, 57%) as an orange solid. R_{f}=0.40 [EtOAc:Hexane 4:1]. ¹H NMR (400 MHz, DMSO) δ=11.00 (s, 1H), 8.10 (dd, J=19.5, 7.8 Hz, 1H), 7.87 (dd, J=15.5, 7.4 Hz, 1H), 7.81-7.61 (m, 1H), 6.89 (s, 2H), 5.15 (dd, J=13.2, 5.1 Hz, 1H), 4.74 (s, 1H), 4.68 (m, 3H), 2.98-2.86 (m, 1H), 2.47 (s, 6H), 2.03 (dd, J=18.4, 13.2 Hz, 1H), 1.83 (d, J=29.9 Hz, 1H), 1.45 (s, 9H) ppm. ¹³C NMR (400 MHz, DMSO) δ=173.36, 171.46, 168.02, 167.76, 158.83, 147.50, 144.68, 135.70, 134.78, 134.14, 130.02, 127.50, 125.28, 115.74, 82.03, 65.44, 52.27, 48.67, 31.70, 28.17, 22.84, 21.05 ppm. HRMS (ESI): calcd. for C₂₇H₃₀N₄O₆Na⁺: 529.2058 m/z [M+Na]⁺; Found: 529.2050 m/z [M+Na]⁺. LCMS (ESI): tᵣₑₜ=4.36 min. 507 m/z [M+H]⁺.

### 2-(4-((2-(2,6-Dioxopiperidin-3-yl)-1-oxoisoindolin-4-yl)diazenyl)-3,5-dimethylphenoxy)acetic acid (KK-39)

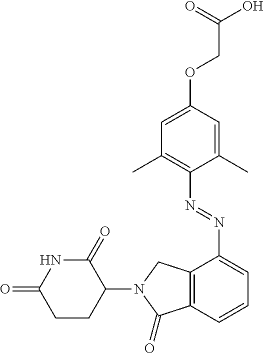 [SMILES: Cc1cc(OCC(=O)O)cc(C)c1/N=N/c1cccc2c1CN(C1CCC(=O)NC1=O)C2=O]

KK-87 (110.0 mg, 217.0 μmol, 1 eq.) was dissolved in a CH₂Cl₂:TFA mixture (1:1; 4 mL). After 4 h the reaction was concentrated under reduced pressure. The mixture was triturated with MeOH and then dried under high vacuum for 48 h. KK-39 (96.00 mg, 213.0 μmol, 98%) was obtained as red solid with traces of residual TFA. R_{f}=0.15 [CH₂Cl₂:MeOH, 9:1]. ¹H NMR (400 MHz, DMSO) δ=11.00 (s, 1H), 8.13 (d, J=7.7 Hz, 1H), 7.89 (d, J=7.4 Hz, 1H), 7.77 (t, J=7.7 Hz, 1H), 6.81 (s, J=6.2 Hz, 2H), 5.15 (dd, J=13.2, 5.0 Hz, 1H), 4.78 (s, 2H), 4.65 (dd, J=31.7, 18.4 Hz, 2H), 2.99-2.85 (m, 1H), 2.70-2.56 (m, 1H), 2.48 (s, 6H), 2.33 (s, 1H), 2.11-1.98 (m, 1H) ppm. ¹³C NMR (400 MHz, DMSO) δ=172.90, 171.00, 167.57, 167.31, 158.37, 147.04, 144.22, 135.25, 134.32, 133.68, 129.57, 127.04, 124.82, 115.29, 81.58, 64.99, 51.81, 48.21, 39.52, 27.71, 20.59 ppm. HRMS (ESI): calcd. for C₂₃H₂₃N₄O₆⁺: 451.1618 m/z [M+H]⁺. Found: 451.1597 m/z [M+H]⁺. LCMS (ESI): tᵣₑₜ=4.16 min. 451 m/z [M+H]⁺.

### tert-Butyl-(4-(2-(4-((2-(2,6-dioxopiperidin-3-yl)-1-oxoisoindolin-4-yl)diazenyl)-3,5-dimethylphenoxy)acetamido)butyl)carbamate (KK-88)

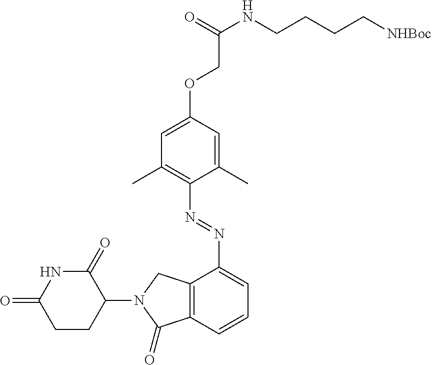 [SMILES: Cc1cc(OCC(=O)NCCCCNC(=O)OC(C)(C)C)cc(C)c1/N=N/c1cccc2c1CN(C1CCC(=O)NC1=O)C2=O]

KK-39 (90.00 mg, 199.8 μmol, 1.0 eq.) and HATU (114.0 mg, 299.7 μmol, 1.5 eq.) were dissolved in dry DMF (5 mL) at room temperature. After 5 minutes of stirring N-Boc-1,4-diaminobutane (75.2 mg, 399.6 μmol, 2.0 eq., 76 μL) and iPr₂NEt (604.0 μmol, 4.0 eq., 105 μL) were added to the mixture and stirred for additional 12 h at room temperature. The reaction was diluted with EtOAc (20 mL), separated against H₂O/10% LiCl (1:1, 10 mL:10 mL), extracted with EtOAc (3×20 mL) and washed with 10% LiCl (3×20 mL) and brine (2×20 mL). The combined organic phase was dried over Na₂SO₄ and concentrated under reduced pressure. Purification of the resulting crude product by flash column chromatography (CH₂Cl₂/MeOH gradient, 0-20% MeOH) gave KK-88 (76.0 mg, 122.4 μmol, 61%) as a yellow solid. R_{f}=0.33 [CH₂Cl₂:MeOH, 19:1]. ¹H NMR (400 MHz, DMSO) δ=10.96 (s, 1H), 8.09 (d, J=7.8 Hz, 1H), 8.07 (d, J=5.9 Hz, 1H), 7.86 (d, J=7.4 Hz, 1H), 7.74 (t, J=7.7 Hz, 1H), 6.81 (s, 2H), 6.74 (t, J=5.3 Hz, 1H), 5.12 (dd, J=13.2, 5.1 Hz, 1H), 4.75-4.57 (m, 2H), 4.51 (s, 2H), 3.09 (q, J=6.2 Hz, 2H), 2.94-2.82 (m, 4H), 2.60 (dd, J=21.9, 11.0 Hz, 1H), 2.45 (s, 6H), 2.01 (dd, J=13.4, 8.2 Hz, 1H), 1.42-1.33 (m, 4H), 1.32 (s, 9H) ppm. ¹³C NMR (400 MHz, CDCl₃) δ=171.24, 171.24, 169.61, 168.75, 167.99, 157.61, 156.22, 147.44, 145.64, 135.61, 134.00, 133.34, 129.57, 128.51, 125.88, 115.55, 67.35, 52.17, 48.44, 38.87, 31.71, 31.06, 28.53, 27.61, 26.91, 23.58, 20.96 ppm. LCMS (ESI): tᵣₑₜ=3.80 min. 565 m/z [M+H-t-Butyl]⁺.

### N-(4-Aminobutyl)-2-(4-((2-(2,6-dioxopiperidin-3-yl)isoindolin-4-yl)diazenyl)-3,5-dimethylphenoxy)acetamide (KK-40)

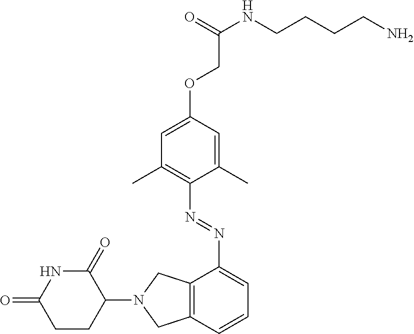 [SMILES: Cc1cc(OCC(=O)NCCCCN)cc(C)c1/N=N/c1cccc2c1CN(C1CCC(=O)NC1=O)C2]

KK-88 (50.00 mg, 80.60 μmol, 1.0 eq.) was dissolved in a CH₂Cl₂:TFA mixture (1:1; 4 mL). After 6 h the reaction was concentrated under reduced pressure. The mixture was triturated with MeOH and then dried under high vacuum for 48 h. KK-40 (41.0 mg, 78.80 μmol, 98%) was obtained as red solid with traces of residual TFA. R_{f}=0.15 [CH₂Cl₂:MeOH, 2:1]. ¹H NMR (400 MHz, DMSO) δ=11.00 (s, 1H), 8.18 (t, J=5.9 Hz, 1H), 8.13 (d, J=7.6 Hz, 1H), 7.90 (d, J=7.4 Hz, 1H), 7.78 (t, J=7.6 Hz, 1H), 7.63 (s, 1H), 6.86 (s, 2H), 5.16 (dd, J=13.3, 5.0 Hz, 1H), 4.69 (dd, J=52.4, 18.5 Hz, 2H), 4.56 (s, 2H), 3.17 (d, J=5.9 Hz, 2H), 2.99-2.95 (m, 2H), 2.80-2.75 (m, 2H), 2.69-2.55 (m, 2H), 2.48 (s, 6H), 2.33 (s, 1H), 2.08-2.01 (m, 1H), 1.85 (d, J=31.7 Hz, 1H), 1.51 (s, 4H) ppm. ¹³C NMR (400 MHz, DMSO) δ=172.93, 171.01, 167.31, 167.29, 158.37, 147.03, 144.30, 135.26, 134.35, 133.68, 129.60, 127.00, 124.89, 115.52, 66.95, 51.83, 48.21, 39.52, 38.59, 37.66, 31.25, 30.71, 26.10, 24.46, 22.42, 20.64 ppm. LCMS (ESI): tᵣₑₜ=2.75 min. 507 m/z [M+H]⁺.

### 2-((S)-4-(4-Chlorophenyl)-2,3,9-trimethyl-6H-thieno[3,2-f][1,2,4]triazolo[4,3-a][1,4]diazepin-6-yl)-N-(4-(2-(4-((2-(2,6-dioxopiperidin-3-yl)-1-oxoisoindolin-4-yl)diazenyl)-3,5-dimethylphenoxy)acetamido)butyl)acetamide (KR-94)

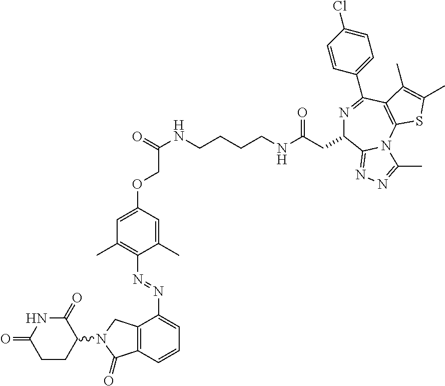

Into a round bottom flask with dry (+)-JQ1 free acid (9.50 mg, 23.70 μmol, 1 eq.) were added KK-40 (27.3 mg, 47.40 μmol, 2 eq.) and HATU (13.5 mg, 35.50 μmol, 1.5 eq.) under nitrogen atmosphere. The solids were dissolved in dry DMF (1 mL). After addition of i-Pr₂NEt (165.9 μmol, 7.0 eq., 29 μL) the reaction was stirred for 15 h at room temperature. The mixture was then diluted with EtOAc (20 mL), separated against 10% LiCl (30 mL), extracted with EtOAc (3×20 mL), washed twice with 10% LiCl (2×20 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) gave KR-94 (14.0 mg, 14.6 μmol, 62%) as an orange solid. R_{f}=0.13 [CH₂Cl₂:MeOH, 19:1]. ¹H NMR (400 MHz, CDCl₃) δ=8.33 (d, J=10.2 Hz, 1H), 8.13 (d, J=7.8 Hz, 1H), 7.97 (d, J=7.5 Hz, 1H), 7.68 (t, J=7.7 Hz, 1H), 7.40 (d, J=8.2 Hz, 2H), 7.33 (d, J=8.3 Hz, 2H), 6.82 (s, 2H), 6.72 (s, 2H), 5.21 (dd, J=13.2, 4.9 Hz, 1H), 4.71 (dd, J=43.7, 17.6 Hz, 2H), 4.69 (s, 1H), 4.53 (s, 2H), 3.64-3.53 (m, 1H), 3.40-3.25 (m, 6H), 2.96-2.74 (m, 2H), 2.67 (s, 3H), 2.47 (s, 6H), 2.39 (s, 3H), 2.27-2.21 (m, 1H), 1.66 (s, 3H), 1.57 (s, 4H) ppm. ¹³C NMR (400 MHz, CDCl₃) δ=171.17, 170.32, 169.55, 168.56, 168.18, 164.55, 155.30, 154.77, 150.31, 150.11, 147.03, 142.83, 137.34, 135.91, 134.09, 133.27, 132.05, 131.53, 131.15, 130.48, 130.06, 129.90, 129.55, 129.42, 128.84, 125.97, 119.29, 72.98, 54.32, 52.05, 48.21, 39.18, 38.96, 38.77, 31.63, 29.70, 27.07, 26.83, 23.94, 23.41, 14.41, 13.15, 11.76 ppm. LCMS (ESI): tᵣₑₜ=4.44 min. 903 m/z [M+H]⁺.

### 3-(4-((4-Hydroxy-2,6-dimethoxyphenyl)diazenyl)-1-oxoisoindolin-2-yl)piperidine-2,6-dione (KK-41)

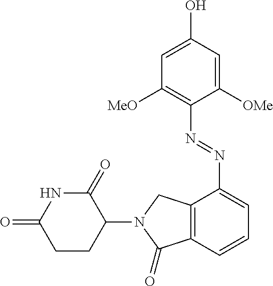 [SMILES: COc1cc(O)cc(OC)c1/N=N/c1cccc2c1CN(C1CCC(=O)NC1=O)C2=O]

Lenalidomide (518.5 mg, 2.000 mmol, 1.0 eq.) was dissolved in 1 M HCl (40 mL) and concentrated aq. HBF₄ (2 mL) was added to the mixture. After completely dissolving of the starting material, 2 M NaNO₂ solution (1.1 mL) was added to the mixture at 0° C. After stirring for 1 h the solution was added dropwise into a mixture of 3,5-Dimethoxyphenol (308.3 mg, 2.000 mmol, 1.0 eq.) in H₂O (50 mL), MeOH (20 mL), NaHCO₃ (4.15 g, 49.37 mmol, 24.7 eq.) and Na₂CO₃ (5.18 g, 49.37 mmol, 24.7 eq.) and stirred for an additional 1 h at 0° C. Filtration and subsequent washing of the solid residue with EtOAc (3×20 mL) gave KK-41 (823.0 mg, 1.939 mmol, 97%) as an orange solid. R_{f}=0.23 [CH₂Cl₂:MeOH, 19:1]. ¹H NMR (400 MHz, DMSO) δ=14.21 (s, 1H), 11.05 (s, 1H), 8.10 (d, J=7.7 Hz, 1H), 7.78 (d, J=7.3 Hz, 1H), 7.71 (t, J=7.6 Hz, 1H), 6.24 (s, J=2.3 Hz, 1H), 6.13 (s, J=2.3 Hz, 1H), 5.16 (dd, J=13.2, 5.1 Hz, 1H), 4.63 (dd, J=52.4, 19.2 Hz, 2H), 3.93 (s, 3H), 3.87 (s, 3H), 3.01-2.87 (m, 1H), 2.66 (d, J=16.6 Hz, 1H), 2.36 (qd, J=13.1, 4.3 Hz, 1H), 2.16-2.05 (m, 1H) ppm. ¹³C NMR (400 MHz, DMSO) δ=172.93, 171.05, 167.25, 166.67, 160.26, 158.11, 144.87, 133.90, 132.63, 129.59, 127.06, 124.16, 123.42, 93.80, 92.03, 56.56, 51.77, 48.72, 39.52, 31.24, 22.71 ppm. HRMS (ESI): calcd. For C₂₁H₂₁N₄O₆⁺: 425.1456 m/z [M+H]⁺. Found: 425.1458 m/z [M+H]⁺. LCMS (ESI): tᵣₑₜ=2.638 min. 425 m/z [M+H]⁺.

### 2-(4-((2-(2,6-dioxopiperidin-3-yl)-1-oxoisoindolin-4-yl)diazenyl)-3,5-dimethoxyphenoxy)acetic acid (KK-42)

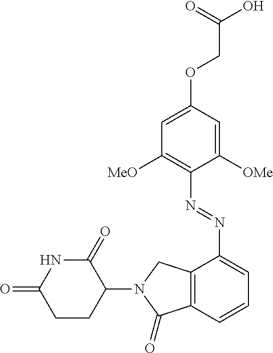 [SMILES: COc1cc(OCC(=O)O)cc(OC)c1/N=N/c1cccc2c1CN(C1CCC(=O)NC1=O)C2=O]

KK-41 (150.0 mg, 353.0 μmol, 1.0 eq.) was dissolved in DMSO (25 mL) at 100° C. The mixture was cooled down to room temperature and tert-Butyl bromoacetate (82.7 mg, 424.0 μmol, 1.2 eq., 63 μL) and iPr₂NEt (707.0 μmol, 2.0 eq., 123 μL) were added. After stirring for 3 h, the mixture was diluted with EtOAc (50 mL), separated against NaHCO₃ (50 mL), extracted with EtOAc (3×50 mL) and washed with 10% LiCl (3×50 mL) and brine (2×50 mL). The reaction was concentrated under reduced pressure and then dissolved in a CH₂Cl₂:TFA mixture (1:1; 4 mL). After 3 h the reaction was concentrated under reduced pressure. Purification of the resulting crude product by flash column chromatography (CH₂Cl₂/MeOH/AcOH (1%) gradient, 0→20% MeOH/AcOH) gave KK-42 (83.0 mg, 0.172 mmol, 49%) as a yellow solid. R_{f}=0.35 [CH₂Cl₂:MeOH, 9:1]. ¹H NMR (400 MHz, DMSO) δ=11.03 (s, 1H), 8.07 (d, J=7.7 Hz, 1H), 7.82 (d, J=7.3 Hz, 1H), 7.74 (t, J=7.5 Hz, 1H), 6.41 (s, 2H), 5.17 (dd, J=13.1, 5.1 Hz, 1H), 4.85 (s, 2H), 4.62-4.48 (m, 3H), 3.83 (s, 6H), 3.0-2.8 (m, 1H), 2.63-2.60 (m, 2H), 2.12-1.98 (m, 1H) ppm. ¹³C NMR (400 MHz, CDCl₃) δ=171.23, 169.56, 168.69, 167.37, 156.11, 155.44, 133.15, 132.42, 131.08, 129.24, 125.12, 92.08, 91.43, 79.31, 67.45, 56.55, 55.80, 51.95, 49.04, 28.41 ppm. HRMS (ESI): calcd. C₂₃H₂₃N₄O₈⁺: 483.1516 m/z [M+H]⁺. Found: 483.1549 m/z [M+H]⁺. LCMS (ESI): tᵣₑₜ=2.625 min. 483 m/z [M+H]⁺.

### N-(4-Aminobutyl)-2-(4-((2-(2,6-dioxopiperidin-3-yl)-1-oxoisoindolin-4-yl)diazenyl)-3,5-dimethoxyphenoxy)acetamide (KK-43)

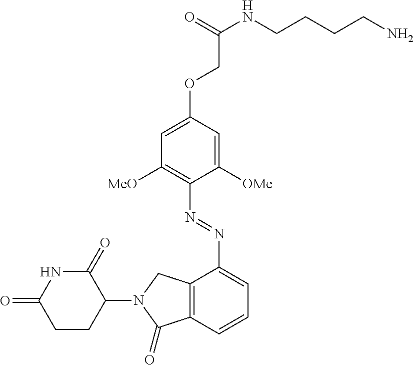 [SMILES: COc1cc(OCC(=O)NCCCCN)cc(OC)c1/N=N/c1cccc2c1CN(C1CCC(=O)NC1=O)C2=O]

KK-42 (29. 0 mg, 60.1 μmol, 1.0 eq.) and HATU (34.3 mg, 120.2 μmol, 2.0 eq.) were dissolved in dry DMF (5 mL) at room temperature. After 5 min of stirring N-Boc-1,4-diaminobutane (22.6 mg, 120.2 μmol, 2.0 eq., 20 μL) and iPr₂NEt (240.4 μmol, 4.0 eq., 40 μL) were added to the mixture and stirred for additional 12 h at room temperature. The reaction was diluted with EtOAc (20 mL), separated against H₂O/10% LiCl (1:1, 10 mL:10 mL), extracted with EtOAc (2×20 mL) and washed with 10% LiCl (2×20 mL) and brine (2×20 mL). The combined organic phase was dried over Na₂SO₄. The reaction was concentrated under reduced pressure and then dissolved in a CH₂Cl₂:TFA mixture (1:1; 4 mL). After 3 h the reaction was concentrated under reduced pressure. Purification of the resulting crude product by flash column chromatography (CH₂Cl₂/MeOH/AcOH (1%) gradient, 0→20% MeOH/AcOH) gave KK-43 (33.9 mg, 0.052 mmol, 87%) as a yellow solid. ¹H NMR (400 MHz, DMSO) δ=11.00 (s, 1H), 8.18 (t, J=5.9 Hz, 1H), 8.13 (d, J=7.6 Hz, 1H), 7.90 (d, J=7.4 Hz, 1H), 7.78 (t, J=7.6 Hz, 1H), 7.63 (s, 1H), 6.86 (s, 2H), 5.16 (dd, J=13.3, 5.0 Hz, 1H), 4.69 (dd, J=52.4, 18.5 Hz, 2H), 4.56 (s, 2H), 3.17 (d, J=5.9 Hz, 2H), 2.99-2.95 (m, 2H), 2.80-2.75 (m, 2H), 2.69-2.55 (m, 2H), 2.48 (s, 6H), 2.33 (s, 1H), 2.08-2.01 (m, 1H), 1.85 (d, J=31.7 Hz, 1H), 1.51 (s, 4H) ppm. ¹³C NMR (400 MHz, CDCl₃) δ=171.17, 170.32, 169.55, 168.56, 168.18, 164.55, 155.30, 154.77, 150.31, 150.11, 147.03, 142.83, 137.34, 135.91, 134.09, 133.27, 132.05, 131.53, 131.15, 130.48, 130.06, 129.90, 129.55, 129.42, 128.84, 125.97, 119.29, 72.98, 54.32, 52.05, 48.21, 39.18, 38.96, 38.77, 31.63, 29.70, 27.07, 26.83, 23.94, 23.41, 14.41, 13.15, 11.76 ppm. HRMS (ESI): calcd. For C₂₇H₃₃N₆O₉⁺: 553.2405 m/z [M+H]⁺. Found: 553.2384 m/z [M+H]⁺. LCMS (ESI): tᵣₑₜ=2.49 min. 553 m/z [M+H]⁺.

### 2-((S)-4-(4-Chlorophenyl)-2,3,9-trimethyl-6H-thieno[3,2-f][1,2,4]triazolo[4,3-a][1,4]diazepin-6-yl)-N-(4-(2-(4-((2-(2,6-dioxopiperidin-3-yl)-1-oxoisoindolin-4-yl)diazenyl)-3,5-dimethoxyphenoxy)acetamido)butyl)acetamide (KR-129)

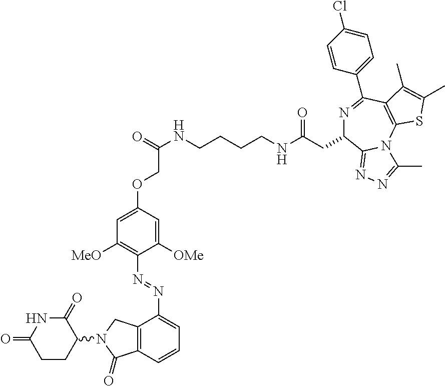

(+)-JQ1 free acid (8.4 mg, 16.80 μmol, 1.0 eq.), KK-43 (9.3 mg, 16.80 mmol, 2.0 eq.) and HATU (9.6 mg, 25.10 μmol, 1.5 eq.) were dissolved in dry DMF (1 mL). After addition of i-Pr₂NEt (117.3 μmol, 7.0 eq., 20 μL) the reaction was stirred for 24 h at room temperature. The mixture was then diluted with EtOAc (20 mL), separated against 5% LiCl (10 mL), extracted with EtOAc (2×10 mL) and washed twice with 10% LiCl (2×10 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) and HPLC gave KR-129 (4.6 mg, 4.90 μmol, 29%) as a red solid. R_{f}=0.13 [CH₂Cl₂:MeOH, 19:1]. ¹H NMR (600 MHz, CDCl₃) δ=8.58-8.39 (m, 1H), 8.21 (d, J=7.7 Hz, 1H), 7.92 (d, J=7.4 Hz, 1H), 7.67 (t, J=7.1 Hz, 1H), 7.39 (d, J=8.0 Hz, 2H), 7.33 (d, J=8.3 Hz, 2H), 6.87 (s, 1H), 6.79 (s, 1H), 6.26 (s, 2H), 5.27-5.21 (m, 1H), 4.71 (dd, J=66.3, 17.6 Hz, 2H), 4.68 (t, J=6.8 Hz, 1H), 4.53 (s, 2H), 3.85 (s, 6H), 3.63-3.54 (m, 3H), 3.37-3.23 (m, 6H), 2.88-2.80 (m, 2H), 2.64 (s, 3H), 2.39 (s, 2H), 1.66 (s, 3H), 1.63-1.47 (m, 4H) ppm. ¹³C NMR (600 MHz, CDCl₃) δ=171.31, 170.58, 169.70, 168.73, 167.52, 164.16, 160.40, 155.37, 148.79, 136.92, 136.52, 133.12, 132.32, 131.38, 131.31, 130.95, 130.47, 129.84, 129.24, 128.79, 127.98, 127.51, 124.97, 122.99, 92.01, 91.50, 67.41, 56.49, 55.82, 54.54, 51.91, 49.13, 45.55, 39.48, 38.98, 38.76, 31.60, 29.73, 26.80, 26.68, 23.58, 14.42, 13.13, 11.83 ppm. LCMS (ESI): tᵣₑₜ=3.60 min. 934 m/z [M+H]⁺.

### 3-(4-((4-hydroxy-2,5-dimethoxyphenyl)diazenyl)-1-oxoisoindolin-2-yl)piperidine-2,6-dione (KK-98)

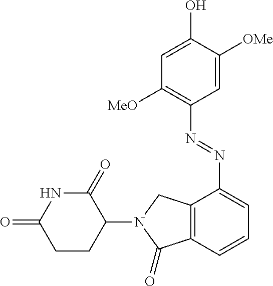 [SMILES: COc1cc(/N=N/c2cccc3c2CN(C2CCC(=O)NC2=O)C3=O)c(OC)cc1O]

Lenalidomide (518 mg, 2.00 mmol, 1.0 eq.) was dissolved in 1 M HCl (50 mL). Concentrated aqueous HBF₄ (2 mL) was added to the mixture. After completely dissolving of the starting material, 2 M NaNO₂ solution (1.10 mL) was added to the solution at 0° C. After stirring for 1 h the solution was added dropwise into a mixture of 2,5-Dimethoxyphenol (357 mg, 2.32 mmol, 1.2 eq.) in H₂O (50 mL), MeOH (20 mL), NaHCO₃ (4.000 g, 47.62 mmol, 24.7 eq.) and Na₂CO₃ (5.000 g, 47.18 mmol, 24.5 eq.) and stirred for 1 h. The reaction was extracted with EtOAc (7×100 mL) and washed once with brine (1×100 mL). The organic phase was dried over Na₂SO₄ and concentrated under reduced pressure. Purification of the resulting crude product by flash column chromatography (CH₂Cl₂/MeOH gradient, 0→10% MeOH) gave KK-98 (197 mg, 0.464 mmol, 23%) as a yellow solid. LCMS (ESI): tᵣₑₜ=2.90 min. 425 m/z [M+H+]⁺.

### tert-Butyl-2-(4-((2-(2,6-dioxopiperidin-3-yl)-1-oxoisoindolin-4-yl)diazenyl)-2,5-dimethoxyphenoxy)acetate (KK-90)

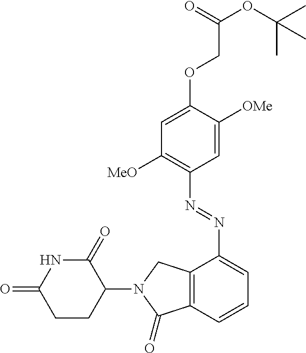 [SMILES: COc1cc(OCC(=O)OC(C)(C)C)c(OC)cc1/N=N/c1cccc2c1CN(C1CCC(=O)NC1=O)C2=O]

To tert-Butyl bromoacetate (195 mg, 0.353 mmol, 0.8 eq., 52 μL) was added dry DMF (10 mL), KK-89 (197 mg, 0.441 mmol, 1.0 eq.) and K2CO₃ (79.2 mg, 0.573 mmol, 1.3 eq.) at room temperature. After stirring for 12 h, the mixture was diluted with EtOAc (50 mL), separated against NaHCO₃ (50 mL), extracted with EtOAc (3×50 mL) and washed with 10% LiCl (3×50 mL) and brine (2×50 mL). The reaction was concentrated under reduced pressure. Purification of the resulting crude product by flash column chromatography (Hexane/EtOAc gradient, 20→100% EtOAc) gave KK-90 (82 mg, 0.152 mmol, 35%) as a yellow solid. LCMS (ESI): tᵣₑₜ=3.93 min. 539 m/z [M+H]⁺.

### tert-Butyl (2-(2-(methyl(phenyl)amino)acetamido)ethyl)carbamate (KK-45)

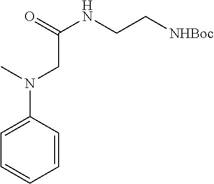 [SMILES: CN(CC(=O)NCCNC(=O)OC(C)(C)C)c1ccccc1]

N-Methyl-N-phenylglycine hydrochloride (200 mg, 0.992 mmol, 1.0 eq.) and HATU (566 mg, 1.488 mmol, 1.5 eq.) were dissolved in dry DMF (5 mL) at room temperature. After 5 min of stirring tert-butyl (2-aminoethyl)carbamate (318 mg, 1.984 mmol, 2.0 eq.) and iPr₂NEt (3.967 mmol, 4.0 eq., 69 μL) were added to the mixture and stirred for further 12 h. The reaction was diluted with EtOAc (20 mL), separated against H₂O and extracted with EtOAc (3×20 mL). The combined organic phase was dried over Na₂SO₄ and concentrated under reduced pressure. KK-45 (301 mg, 0.979 mmol, 99%) was obtained as white solid. R_{f}=0.20 [Hexane: EtOAc, 4:1]. ¹H NMR (400 MHz, CDCl₃) δ=7.29 (d, J=8.6 Hz, 2H), 7.03 (s, 1H), 6.85 (t, J=7.3 Hz, 1H), 6.74 (d, J=8.1 Hz, 2H), 4.79 (s, 1H), 3.87 (s, 2H), 3.39 (dd, J=11.6, 5.8 Hz, 2H), 3.29-3.18 (m, 2H), 3.04 (s, 3H), 1.41 (s, 9H) ppm. ¹³C NMR (400 MHz, CDCl₃) δ=171.35, 149.32, 129.52, 118.75, 113.22, 77.16, 58.88, 40.59, 40.00, 38.74, 31.06, 28.46 ppm. HRMS (ESI): calcd. For C₁₆H₂₅N₃O₃Na⁺: 330.1690 m/z [M+Na]⁺. Found: 330.1751 m/z [M+Na]⁺. LCMS (ESI): tᵣₑₜ=3.49 min. 252 m/z [M+H-t-Butyl]⁺.

### tert-Butyl-(2-(2-((4-((2-(2,6-dioxopiperidin-3-yl)-1-oxoisoindolin-4-yl)diazenyl)phenyl)(methyl)amino)acetamido)ethyl)carbamate (KK-46)

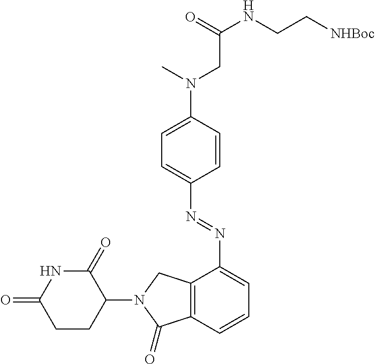 [SMILES: CN(CC(=O)NCCNC(=O)OC(C)(C)C)c1ccc(/N=N/c2cccc3c2CN(C2CCC(=O)NC2=O)C3=O)cc1]

Lenalidomide (220 mg, 849.0 μmol, 1.0 eq.) was dissolved in 1 M HCl (20 mL). Concentrated aq. HBF₄ (1 mL) was added to the mixture. After completely dissolving of the starting material, 2 M NaNO₂ solution (470 μL) was added to the mixture at 0° C. After stirring for 1 h the solution was added dropwise into a mixture of KK-45 (261 mg, 849 mg, 1.0 eq.) in H₂O (20 mL), MeOH (5 mL), NaOAc (1.74 g, 21.21 mmol, 25.0 eq.). The mixture was stirred for 1 h at 0° C. MeOH was removed under reduced pressure, the aqueous phase extracted with EtOAc (3×30 mL) and washed with brine (2×30 mL). The organic phase was dried over Na₂SO₄ and concentrated under reduced pressure. Purification of the resulting crude product by flash column chromatography (CH₂Cl₂/MeOH gradient, 0→10% MeOH) gave KK-46 (114 mg, 197 μmol, 23%) as an orange solid. R_{f}=0.13 [CH₂Cl₂:MeOH, 19:1]. ¹H NMR (400 MHz, DMSO) δ=11.01 (s, 1H), 8.1-8.0 (m, 2H), 7.85 (d, J=8.9 Hz, 2H), 7.79 (d, J=7.4 Hz, 1H), 7.71 (t, J=7.6 Hz, 1H), 6.78 (d, J=8.9 Hz, 2H), 5.16 (dd, J=13.1, 4.9 Hz, 1H), 4.71 (dd, J=49.6, 18.9 Hz, 2H), 4.06 (s, 2H), 3.20-3.05 (m, J=7.8 Hz, 6H), 3.04-2.86 (m, 3H), 2.58 (dd, J=24.5, 11.0 Hz, 2H), 2.12-1.98 (m, 1H), 1.37 (s, 9H) ppm. ¹³C NMR (400 MHz, DMSO) δ=182.40, 180.53, 178.25, 176.91, 165.14, 161.95, 156.56, 152.66, 143.46, 143.07, 138.88, 136.83, 134.40, 133.11, 121.16, 87.15, 64.82, 61.14, 57.70, 49.00, 40.73, 37.69, 31.82 ppm. HRMS (ESI): calcd. For C₂₉H₃₆N₇O₆⁺: 578.2649 m/z [M+H]⁺. Found: 578.2682. LCMS (ESI): tᵣₑₜ=3.44 min. 522 m/z [M+H-t-butyl]⁺.

### N-(4-Aminobutyl)-2-((4-((2-(2,6-dioxopiperidin-3-yl)-1-oxoisoindolin-4-yl)diazenyl)phenyl)(methyl)amino)acetamide (KK-47)

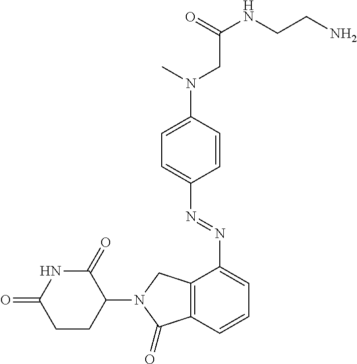 [SMILES: CN(CC(=O)NCCN)c1ccc(/N=N/c2cccc3c2CN(C2CCC(=O)NC2=O)C3=O)cc1]

KK-46 (30.0 mg, 0.050 mmol, 1.0 eq.) was dissolved in a CH₂Cl₂:TFA mixture (1:1; 4 mL). After 4 h the reaction was concentrated under reduced pressure and triturated with MeOH. KK-47 (27.0 mg, 0.049 mmol, 99%) was obtained as red solid with traces of residual TFA. R_{f}=0.15 [CH₂Cl₂:MeOH, 1:1]. ¹H NMR (400 MHz, DMSO) δ=11.01 (s, 1H), 8.06 (s, 1H), 7.87 (d, J=8.8 Hz, 2H), 7.81 (d, J=7.4 Hz, 1H), 7.73 (t, J=7.7 Hz, 1H), 7.67 (d, J=19.4 Hz, 3H), 6.79 (d, J=8.9 Hz, 2H), 5.17 (dd, J=13.1, 4.9 Hz, 1H), 4.70 (dt, J=38.8, 12.6 Hz, 2H), 4.08 (s, 2H), 3.13 (s, 3H), 3.12-3.07 (m, 2H), 2.94 (dd, J=21.4, 8.7 Hz, 1H), 2.79 (dd, J=12.4, 6.2 Hz, 2H), 2.68-2.55 (m, 2H), 2.08-2.01 (m, 1H), 1.54-1.43 (m, 4H) ppm. ¹³C NMR (400 MHz, DMSO) δ=172.97, 171.10, 168.67, 167.46, 152.50, 147.10, 143.21, 134.03, 133.64, 129.43, 127.34, 124.95, 123.70, 111.68, 55.33, 55.08, 51.70, 48.25, 38.57, 37.89, 31.29, 26.14, 24.49, 22.38 ppm. HRMS (ESI): calcd. For C₂₄H₂₇N₇O₄⁺: 501.1756 m/z [M+H]⁺. Found: 501.1762 m/z [M+H]⁺. LCMS (ESI): tᵣₑₜ=2.54 min. 501 m/z [M+H]⁺.

### 2-((S)-4-(4-Chlorophenyl)-2,3,9-trimethyl-6H-thieno[3,2-f][1,2,4]triazolo[4,3-a][1,4]diazepin-6-yl)-N-(2-(2-((4-((2-(2,6-dioxopiperidin-3-yl)-1-oxoisoindolin-4-yl)diazenyl)phenyl)(methyl)amino)acetamido)ethyl)acetamide (KR-114)

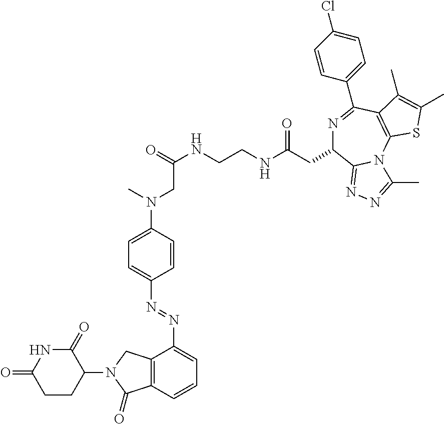

(+)-JQ1 free acid (7.02 mg, 17.50 μmol, 1.0 eq.), KK-47 (16.73 mg, 35.00 mmol, 2.0 eq.) and HATU (10.0 mg, 26.30 μmol, 2 eq.) were dissolved in dry DMF (1 mL). After addition of i-Pr₂NEt (122.5 μmol, 7.0 eq., 21 μL) the reaction was stirred for 24 h at room temperature. The mixture was then diluted with EtOAc (20 mL), separated against 5% LiCl (30 mL), extracted with EtOAc (2×20 mL) and washed twice with 10% LiCl (2×20 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) gave KR-114 (15.0 mg, 17.10 μmol, 98%) as a red solid. R_{f}=0.07 [CH₂Cl₂:MeOH, 19:1]. ¹H NMR (400 MHz, DMSO) δ=11.00 (s, 1H), 8.23 (s, 1H), 8.04 (d, J=7.7 Hz, 2H), 7.83 (dd, J=9.2, 2.5 Hz, 2H), 7.79 (d, J=7.5 Hz, 1H), 7.70 (t, J=7.6 Hz, 1H), 7.46-7.42 (m, 2H), 7.41-7.37 (m, 2H), 6.77 (d, J=7.7 Hz, 2H), 5.19-5.10 (m, 1H), 4.70 (dd, J=46.1, 19.3 Hz, 2H), 4.48 (td, J=7.3, 2.7 Hz, 1H), 4.07 (s, J=13.8 Hz, 2H), 3.19 (s, J=2.4 Hz, 6H), 3.12 (s, 3H), 2.99-2.86 (m, 1H), 2.56 (d, J=4.0 Hz, 4H), 2.39 (s, 3H), 2.06-1.98 (m, 1H), 1.60 (s, 3H), 1.23 (s, 1H) ppm. ¹³C NMR (400 MHz, DMSO) δ=172.92, 171.05, 169.88, 168.87, 167.44, 163.07, 155.07, 152.43, 149.81, 147.08, 143.20, 136.71, 135.19, 134.05, 133.60, 132.23, 130.66, 130.11, 129.84, 129.53, 129.35, 128.44, 127.28, 124.88, 123.61, 111.68, 55.41, 53.74, 51.70, 48.28, 39.52, 38.22, 37.64, 31.25, 30.70, 22.34, 14.05, 12.66, 11.26 ppm. LCMS (ESI): tᵣₑₜ=3.60 min. 860 m/z [M+H]⁺.

### tert-Butyl (4-(2-(methyl(phenyl)amino)acetamido)butyl)carbamate (KK-48)

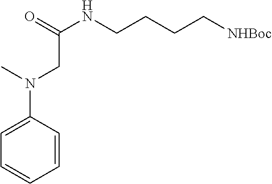 [SMILES: CN(CC(=O)NCCCCNC(=O)OC(C)(C)C)c1ccccc1]

N-Methyl-N-phenylglycine hydrochloride (202 mg, 1.000 mmol, 1.0 eq.) and HATU (570 mg, 1.500 mmol, 1.5 eq.) were dissolved in dry DMF (5 mL) at room temperature. After 5 min of stirring tert-Butyl (4-aminobutyl)carbamate (377 mg, 2.000 mmol, 2.0 eq., 285 μL) and i-Pr₂NEt (4.000 mmol, 4.0 eq., 700 μL) were added to the mixture and stirred for further 12 h. The reaction was diluted with EtOAc (20 mL), separated against H₂O and extracted with EtOAc (3×20 mL). The combined organic phase was dried over Na₂SO₄ and concentrated under reduced pressure. KK-48 (306 mg, 0.912 mmol, 91%) was obtained as off-white solid. R_{f}=0.23 [Hexane: EtOAc, 4:1]. ¹H NMR (400 MHz, CDCl₃) δ=7.27 (dd, J=10.4, 5.0 Hz, 2H), 6.84 (dd, J=16.7, 9.4 Hz, 1H), 6.74 (d, J=8.1 Hz, 2H), 6.66 (s, 1H), 4.52 (s, 1H), 3.85 (s, 2H), 3.28 (q, J=6.5 Hz, 2H), 3.12-3.04 (m, 2H), 3.01 (s, 3H), 1.53-1.37 (m, 4H), 1.43 (s, 9H) ppm. ¹³C NMR (400 MHz, CDCl₃) δ=170.32, 156.07, 149.23, 129.54, 118.99, 113.40, 79.25, 59.05, 40.19, 40.09, 38.91, 28.53, 27.48, 27.00 ppm. HRMS (ESI): calcd. For C₁₈H₃₀N₃O₃⁺: 336.2282 m/z [M+H]⁺. Found: 336.2242 m/z [M+H]⁺. LCMS (ESI): tᵣₑₜ=3.59 min. 336 m/z [M+H]⁺.

### tert-Butyl-(4-(2-((4-((2-(2,6-dioxopiperidin-3-yl)-1-oxoisoindolin-4-yl)diazenyl)phenyl)(methyl)amino)acetamido)butyl)carbamate (KK-49)

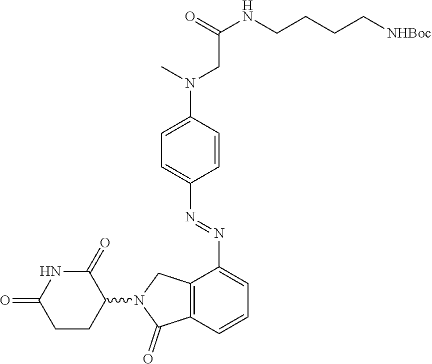 [SMILES: CN(CC(=O)NCCCCNC(=O)OC(C)(C)C)c1ccc(/N=N/c2cccc3c2CN(C2CCC(=O)NC2=O)C3=O)cc1]

Lenalidomide (173 mg, 0.668 mmol, 1.0 eq.) was dissolved in 1 M HCl (20 mL). Concentrated aqueous HBF₄ (1 mL) was added to the mixture. After completely dissolving of the starting material, 2 M NaNO₂ solution (370 μL) was added to the mixture at 0° C. After stirring for 1 h the solution was added dropwise into a mixture of KK-48 (224.0 mg, 0.668 mg, 1.0 eq.) in H₂O (20 mL), MeOH (5 mL), NaOAc (1.10 g, 13.36 mmol, 20 eq.). The mixture was stirred for 1 h at 0° C. MeOH was removed under reduced pressure, the aqueous phase extracted with EtOAc (3×30 mL) and washed once with brine (1×30 mL). The organic phase was dried over Na₂SO₄ and concentrated under reduced pressure. Purification of the resulting crude product by flash column chromatography (CH₂Cl₂/MeOH gradient, 0→10% MeOH) gave KK-49 (165 mg, 0.272 mmol, 41%) as a yellow solid. R_{f}=0.15 [CH₂Cl₂:MeOH, 19:1]. ¹H NMR (400 MHz, CDCl₃) δ=7.27 (dd, J=10.4, 5.0 Hz, 2H), 6.84 (dd, J=16.7, 9.4 Hz, 1H), 6.74 (d, J=8.1 Hz, 2H), 6.66 (s, 1H), 4.52 (s, 1H), 3.85 (s, 2H), 3.28 (q, J=6.5 Hz, 2H), 3.12-3.04 (m, 2H), 3.01 (s, 3H), 1.53-1.37 (m, 4H), 1.43 (s, 9H) ppm. ¹³C NMR (400 MHz, DMSO) δ=172.94, 171.07, 168.45, 167.46, 155.59, 152.50, 147.10, 143.18, 134.05, 133.62, 129.39, 127.26, 124.92, 123.63, 111.64, 77.35, 55.29, 54.92, 51.70, 48.26, 38.29, 31.27, 28.28, 26.96, 26.51, 22.35 ppm. HRMS (ESI): calcd. For C₃₁H₃₉N₇O₆⁺: 628.2854 m/z [M+Na]⁺. Found: 628.2883 m/z [M+Na]⁺. LCMS (ESI): tᵣₑₜ=3.61 min. 628 m/z [M+Na]⁺.

### N-(4-aminobutyl)-2-((4-((2-(2,6-dioxopiperidin-3-yl)-1-oxoisoindolin-4-yl)diazenyl)phenyl)(methyl)amino)acetamide (KK-50)

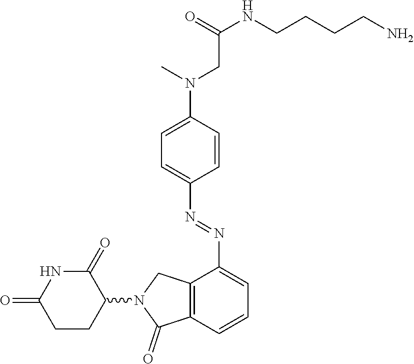 [SMILES: CN(CC(=O)NCCCCN)c1ccc(/N=N/c2cccc3c2CN(C2CCC(=O)NC2=O)C3=O)cc1]

KK-49 (30.0 mg, 0.050 mmol, 1 eq.) was dissolved in a CH₂Cl₂:TFA mixture (1:1; 4 mL). After 4 h the reaction was concentrated under reduced pressure. The mixture was triturated with MeOH and then dried under high vacuum for 48 h. KK-50 (27.0 mg, 0.049 mmol, 99%) was obtained as a red solid with traces of residual TFA. R_{f}=0.15 [CH₂Cl₂:MeOH, 1:1]. ¹H NMR (400 MHz, DMSO) δ=11.01 (s, 1H), 8.06 (s, 1H), 7.87 (d, J=8.8 Hz, 2H), 7.81 (d, J=7.4 Hz, 1H), 7.73 (t, J=7.7 Hz, 1H), 7.67 (d, J=19.4 Hz, 3H), 6.79 (d, J=8.9 Hz, 2H), 5.17 (dd, J=13.1, 4.9 Hz, 1H), 4.70 (dt, J=38.8, 12.6 Hz, 2H), 4.08 (s, 2H), 3.13 (s, 3H), 3.12-3.07 (m, 2H), 2.94 (dd, J=21.4, 8.7 Hz, 1H), 2.79 (dd, J=12.4, 6.2 Hz, 2H), 2.68-2.55 (m, 2H), 2.08-2.01 (m, 1H), 1.54-1.43 (m, 4H) ppm. ¹³C NMR (400 MHz, DMSO) δ=172.97, 171.10, 168.67, 167.46, 152.50, 147.10, 143.21, 134.03, 133.64, 129.43, 127.34, 124.95, 123.70, 111.68, 55.33, 55.08, 51.70, 48.25, 38.57, 37.89, 31.29, 26.14, 24.49, 22.38 ppm. HRMS (ESI): calcd. For C₂₆H₃₂N₇O₄⁺: 506.2510 m/z [M+H]⁺. Found: 506.2471 m/z [M+H]⁺. LCMS (ESI): tᵣₑₜ=2.46 min. 506 m/z [M+H]⁺.

### 2-((S)-4-(4-Chlorophenyl)-2,3,9-trimethyl-6H-thieno[3,2-f][1,2,4]triazolo[4,3-a][1,4]diazepin-6-yl)-N-(4-(2-((4-((2-(2,6-dioxopiperidin-3-yl)-1-oxoisoindolin-4-yl)diazenyl)phenyl)(methyl)amino)acetamido)butyl)acetamide (KR-85)

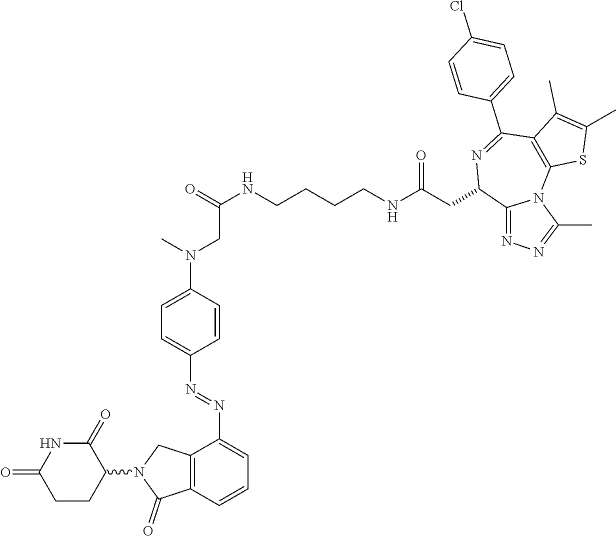

Into a round bottom flask with dry (+)-JQ1 free acid (9.0 mg, 22.50 μmol, 1.0 eq.) were added KK-50 (22.7 mg, 44.90 mmol, 2.0 eq.) and HATU (12.8 mg, 33.7 μmol, 1.5 eq.) under nitrogen atmosphere. The solids were dissolved in dry DMF (1 mL). After addition of i-Pr₂NEt (157.2 μmol, 7.0 eq., 27 μL) the reaction was stirred for 48 h at room temperature. The mixture was then diluted with EtOAc (20 mL), separated against 5% LiCl (30 mL), extracted with EtOAc (2×20 mL), washed twice with 10% LiCl (2×20 mL) and brine (2×20 mL). The reaction was dried over Na₂SO₄ and concentrated under reduced pressure. Purification of the resulting crude product by flash column chromatography (CH₂Cl₂/MeOH gradient, 0→20% MeOH) gave KR-85 (7.00 mg, 7.90 μmol, 35%) as a red solid. R_{f}=0.07 [CH₂Cl₂:MeOH, 19:1]. LCMS (ESI): tᵣₑₜ=3.81 min. 888 m/z [M+H]⁺.

### Ethyl 5-amino-1,3-dioxo-6-((trimethylsilyl)ethynyl)isoindoline-2-carboxylate (KK-57)

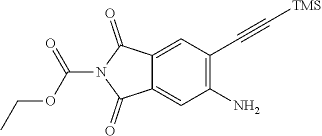 [SMILES: CCOC(=O)N1C(=O)c2cc(N)c(C#C[Si](C)(C)C)cc2C1=O]

MB-90 (1.03 g, 2.866 mmol, 1.0 eq.), CuI (27.3 mg, 0.143 mmol, 0.05 eq.) and Pd(PPh₃)₂Cl₂ (100.6 mg, 0.143 mmol, 0.05 eq.) were dissolved in dry THF (18 mL) at room temperature. TMS acetylene (844.4 mg, 8.597 mmol, 3.0 eq., 1.2 mL) was added to the mixture and NEt₃ (870.0 mg, 8.597 mmol, 3.0 eq., 1.2 mL) was added last. After 7 h of stirring the reaction was diluted with EtOAc (25 mL), filtered through Celite and washed with EtOAc (50 mL). The reaction was concentrated under reduced pressure. Purification of the resulting crude product by flash column chromatography (Hexane/EtOAc gradient, 0→100% EtOAc) gave KK-57 (523.0 mg, 1.583 mmol, 55%) as yellow crystals. R_{f}=0.33 [Hexane:EtOAc, 2:1]. ¹H NMR (400 MHz, DMSO) δ=7.64 (s, 1H), 7.15 (s, 1H), 6.76 (s, 2H), 4.32 (q, J=7.1 Hz, 2H), 1.29 (t, J=7.1 Hz, 3H), 0.27 (s, 9H) ppm. ¹³C NMR (400 MHz, CDCl₃) δ=163.99, 163.20, 153.95, 148.88, 132.94, 129.25, 119.04, 113.32, 107.95, 99.23, 77.36, 63.98, 14.28, 0.00 ppm. HRMS (APCI): calcd. for C₁₆H₁₉N₂O₄SiNa⁺: 353.0928 m/z [M+Na]⁺; found: 353.1201 m/z [M+Na]⁺. LCMS (ESI): tᵣₑₜ=4.43 min. 353 m/z [M+Na]⁺.

### Ethyl 5-amino-6-ethynyl-1,3-dioxoisoindoline-2-carboxylate (KK-58)

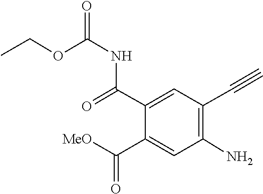 [SMILES: C#Cc1cc(C(=O)NC(=O)OCC)c(C(=O)OC)cc1N]

KK-57 (200.0 mg, 605.3 μmol, 1.0 eq.) was dissolved in dry MeOH (5 mL) and K2CO₃ (167.3 mg, 1.211 mmol, 2.0 eq) was added. After 10 min of stirring the reaction was diluted with H₂O (25 mL), extracted with CH₂Cl₂ (3×25 mL) and EtOAc (3×25 mL) and washed twice with brine (2×30 mL). The organic phase was dried over Na₂SO₄ and concentrated under reduced pressure. KK-58 (95.0 mg, 367.9 μmol, 61%) was obtained as a light orange crystalline solid. R_{f}=0.50 [EtOAc:Hexane, 2:1]. ¹H NMR (400 MHz, DMSO) δ=10.93 (d, J=23.0 Hz, 1H), 7.71 (s, 1H), 6.55 (s, 1H), 6.35 (s, 2H), 4.47 (s, 1H), 4.04 (q, J=7.1 Hz, 2H), 3.70 (s, 3H), 1.16 (t, J=7.1 Hz, 3H) ppm. ¹³C NMR (400 MHz, DMSO) δ=164.65, 153.42, 151.57, 140.67, 134.44, 113.22, 111.38, 104.33, 86.36, 79.19, 60.99, 51.64, 39.52, 14.13 ppm. HRMS (APCI): calcd. for C₁₄H₁₄N₂NaO₅⁺: 313.0807 m/z [M+Na]⁺. found: 313.0851 m/z [M+Na]⁺. LCMS (ESI): tᵣₑₜ=2.64 min. 313 m/z [M+Na]⁺.

### Methyl 5-amino-4-(3-(4-(tert-butoxycarbonyl)-2-nitrophenyl)prop-1-yn-1-yl)-2-((ethoxycarbonyl)carbamoyl)benzoate (KK-61)

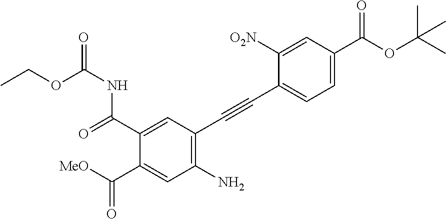 [SMILES: CCOC(=O)NC(=O)c1cc(C#Cc2ccc(C(=O)OC(C)(C)C)cc2[N+](=O)[O-])c(N)cc1C(=O)OC]

MB-18 (140.0 mg, 482.3 μmol, 1.0 eq.), KK-58 (84.2 mg, 241.2 μmol, 1.0 eq.) and NEt₃ (73.2 mg, 723.5 μmol, 1.5 eq., 0.1 mL) were suspended in dry THF (5 mL) under nitrogen. CuI (2.3 mg, 12.10 μmol, 0.025 eq.) and PdCl₂(PPh₃)₂ (8.5 mg, 12.10 μmol, 0.025 eq.) was added last. After 24 h of stirring the reaction was extracted with CH₂Cl₂ (3×25 mL) and EtOAc (3×25 mL) and washed twice with brine (2×30 mL). Purification of the resulting crude product by flash column chromatography (CH₂Cl₂/MeOH gradient, 0→2% MeOH) gave KK-61 (127.0 mg, 248.3 mmol, 52%) as orange solid. R_{f}=0.17 [CH₂Cl₂:MeOH, 19:1]. ¹H NMR (400 MHz, DMSO) δ=10.97 (s, 1H), 8.55 (d, J=1.5 Hz, 1H), 8.23 (dd, J=8.1, 1.6 Hz, 1H), 8.15 (d, J=8.1 Hz, 1H), 7.87 (s, 1H), 6.68 (s, 2H), 6.62 (s, 1H), 4.05 (q, J=7.1 Hz, 2H), 3.73 (s, J=6.5 Hz, 3H), 1.58 (s, 9H), 1.16 (t, J=7.1 Hz, 3H) ppm. ¹³C NMR (400 MHz, DMSO) δ=164.51, 162.74, 153.71, 151.59, 148.29, 141.77, 135.12, 134.93, 133.31, 131.39, 125.24, 121.40, 113.79, 111.80, 103.47, 95.76, 90.60, 82.46, 61.05, 51.76, 39.52, 27.67, 14.11 ppm. HRMS (APCI): calcd. for C₂₅H₂₅N₃NaO₉⁺: 534.1483 m/z [M+Na]⁺; found: 534.1423 m/z [M+Na]⁺. LCMS (ESI): tᵣₑₜ=4.60 min. 534 m/z [M+Na]⁺.

### Methyl 5-amino-4-(2-amino-4-(tert-butoxycarbonyl)phenethyl)-2-((ethoxycarbonyl)carbamoyl)benzoate (KK-62)

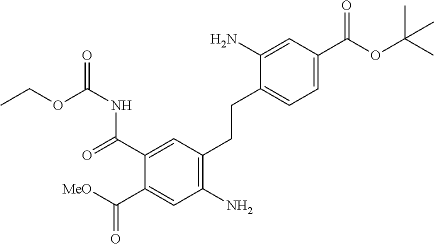 [SMILES: CCOC(=O)NC(=O)c1cc(CCc2ccc(C(=O)OC(C)(C)C)cc2N)c(N)cc1C(=O)OC]

KK-61 (120.0 mg, 250.3 μmol, 1.0 eq.) and 10% Pd/C (26.60 mg, 25.00 μmol, 0.1 eq.) were dissolved in dry MeOH (5 ml) and CH₂Cl₂ (3 mL) under nitrogen. The flask was then charged with H2 and the reaction mixture was stirred for 24 h. The reaction was filtered over Celite, extracted in EtOAc (3×25 mL), washed twice with brine (2×30 mL) and concentrated under reduced pressure. KK-62 (93.0 mg, 205.1 μmol, 82%) was obtained as a yellow oil. R_{f}=0.17 [CH₂Cl₂:MeOH, 9:1]. ¹H NMR (400 MHz, CDCl₃) δ=7.82 (s, 1H), 7.74 (s, 1H), 7.36 (dd, J=7.8, 1.5 Hz, 1H), 7.30 (s, J=1.3 Hz, 1H), 7.05 (d, J=7.8 Hz, 1H), 6.53 (s, 1H), 4.14 (t, J=14.4, 7.2 Hz, 2H), 3.82 (s, 3H), 2.87-2.76 (m, 4H), 1.57 (s, 9H), 1.24 (t, J=7.3 Hz, 3H) ppm. ¹³C NMR (400 MHz, CDCl₃) δ=168.93, 165.38, 164.60, 157.90, 154.95, 151.01, 136.42, 131.97, 131.81, 131.43, 129.95, 129.74, 128.87, 126.63, 120.40, 118.11, 81.71, 62.65, 52.72, 31.69, 31.47, 28.26, 14.17 ppm. HRMS (APCI): calcd. for C₂₁H₂₄N₃O₇⁺: 486.2235 m/z [M+H]⁺; found:486.2232 m/z [M+H]⁺; LCMS (ESI): tᵣₑₜ=3.77 min. 430 m/z [M+H−tBu]⁺.

### 8-(tert-Butyl) 3-methyl-2-((ethoxycarbonyl)carbamoyl)-11,12-dihydrodibenzo[c,g][1,2]diazocine-3,8-dicarboxylate (KK-63)

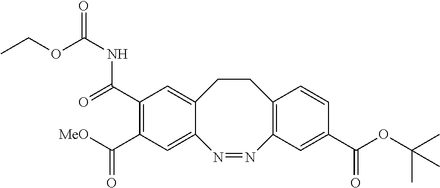 [SMILES: CCOC(=O)NC(=O)c1cc2c(cc1C(=O)OC)/N=N\c1cc(C(=O)OC(C)(C)C)ccc1CC2]

KK-62 (53.0 mg, 0.109 mmol, 1 eq.) was dissolved in a mixture of CH₂Cl₂ (6 mL) and AcOH (6 mL). 3-Chloroperoxybenzoic acid (75%, 50.2 mg, 0.218 mmol) was diluted in AcOH (2 mL) and added dropwise over 14 h at room temperature. The reaction was stirred for additional 12 h at 55° C. and the volatiles were removed under reduced pressure. The residue was dissolved in CH₂Cl₂ (10 mL), separated against NaHCO₃ (10 mL) and extracted with CH₂Cl₂ (3×10 mL). The combined organic phase was washed with brine (2×30 mL), dried over Na₂SO₄ and the solvent was removed under reduced pressure. Purification of the resulting crude product by flash column chromatography (CH₂Cl₂/MeOH gradient, 0→20%) gave KK-63 (38.7 mg, 0.080 mmol, 74%) as a yellow sticky solid. R_{f}=0.35 [CH₂Cl₂:MeOH, 19:1]. ¹H NMR (400 MHz, CDCl₃) δ=7.73 (s, 1H), 7.67 (dd, J=4.0 Hz, 1H), 7.65, (s, 1H), 7.06 (d, J=8.0 Hz, 1H), 6.88 (s, 1H), 4.12-4.00 (m, 2H), 3.82 (s, 3H), 3.10-2.97 (m, 3H), 2.96-2.80 (m, 3H), 1.55 (s, 9H), 1.16 (t, J=7.1 Hz, 3H) ppm. ¹³C NMR (400 MHz, CDCl₃) δ=168.93, 165.38, 164.60, 157.90, 154.95, 151.01, 136.42, 131.97, 131.81, 131.43, 129.95, 129.74, 128.87, 126.63, 120.40, 118.11, 81.71, 62.65, 52.72, 31.69, 31.47, 28.26, 14.17 ppm. LCMS (ESI): tᵣₑₜ=4.31 min. 504 m/z [M+Na]⁺.

### tert-Butyl-2-(2,6-dioxopiperidin-3-yl)-1,3-dioxo-2,3,11,12-tetrahydro-1H-benzo[7,8][1,2]diazocino[3,4-f]isoindole-8-carboxylate (KK-65)

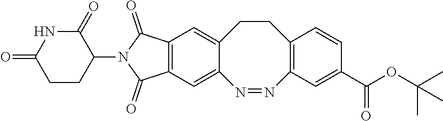 [SMILES: CC(C)(C)OC(=O)c1ccc2c(c1)/N=N\c1cc3c(cc1CC2)C(=O)N(C1CCC(=O)NC1=O)C3=O]

KK-63 (13.9 mg, 28.80 μmol, 1.0 eq.), NaOAc (7.10 mg, 86.50 μmol, 3.0 eq.) and 3-aminopiperidine-2,6-dione (7.1 mg, 43.30 μmol, 1.5 eq.) were dissolved in dry MeCN (3 mL) and stirred for 24 h at 82° C. The mixture was diluted with EtOAc (5 mL), separated against NaHCO₃ (10 mL) and extracted with EtOAc (3×10 mL). The combined organic phase was washed with brine (2×10 mL), dried over Na₂SO₄ and the solvent was under reduced pressure. Purification of the resulting crude product by flash column chromatography (CH₂Cl₂/MeOH gradient, 0→20% MeOH) gave KK-65 (7.1 mg, 14.3 μmol, 50%) as a yellow solid. R_{f}=0.16 [CH₂Cl₂:MeOH, 19:1]. ¹H NMR (400 MHz, CDCl₃) δ=8.05 (s, 1H), 7.68 (d, J=7.9 Hz, 1H), 7.55 (s, J=2.0 Hz, 1H), 7.47 (s, 1H), 7.33 (s, J=1.4 Hz, 1H), 7.02 (d, J=8.0 Hz, 1H), 4.91 (dd, J=12.2, 5.4 Hz, 1H), 3.21-2.66 (m, 7H), 2.15-2.07 (m, 1H), 1.56 (s, 9H) ppm. ¹³C NMR (400 MHz, CDCl₃) δ=170.73, 167.87, 166.29, 164.51, 160.32, 154.82, 135.67, 131.73, 131.33, 131.01, 130.48, 129.07, 125.20, 120.21, 114.49, 81.95, 49.57, 31.47, 29.86, 28.27, 22.70, 14.36 ppm. HRMS (APCI): calcd. for C₂₆H₂₅N₄O₆⁺: 488.1880 m/z [M+H]⁺; found:488.1696 m/z [M+H]⁺. LCMS (ESI): tᵣₑₜ=4.17 min. 511 m/z [M+Na]⁺.

### tert-Butyl (S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate (KK-66)

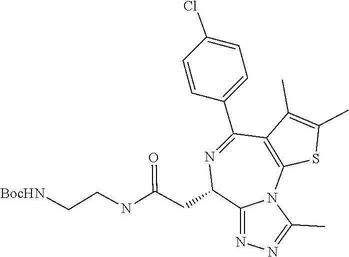

(+)-JQ1 free acid (13.2 mg, 32.9 μmol, 1.0 eq.) and HATU (18.8 mg, 49.4 μmol, 1.5 eq.) were dissolved in dry DMF (1 mL) at room temperature. After 5 min of stirring N-Boc-1,4-diaminobutane (9.84 mg, 65.9 μmol, 2.0 eq., 10 μL) and i-Pr₂NEt (132 μmol, 4.0 eq., 23 μL) were added to the mixture and it was stirred for additional 12 h at room temperature. The reaction was diluted with EtOAc (5 mL), separated against H₂O/10% LiCl (1:1, 5 mL:5 mL), extracted with EtOAc (2×5 mL) and washed with 10% LiCl (2×5 mL) and brine (2×5 mL). The combined organic phase was dried over Na₂SO₄ and concentrated under reduced pressure. Purification of the resulting crude product by flash column chromatography (CH₂Cl₂/MeOH gradient, 0-10% MeOH) gave KK-66 (13.4 mg, 24.7 μmol, 75%) as a yellow solid. R_{f}=0.17 [CH₂Cl₂, MeOH 19:1]. ¹H NMR (400 MHz, CDCl₃) δ=7.39 (d, J=8.5 Hz, 2H), 7.35 (s, 1H), 7.31 (d, J=8.7 Hz, 2H), 5.43 (s, 1H), 4.66 (t, J=7.0 Hz, 1H), 3.55 (dd, J=14.6, 7.5 Hz, 1H), 3.49-3.19 (m, 5H), 2.67 (s, 3H), 2.40 (s, 3H), 1.67 (s, 3H), 1.41 (s, J=13.5 Hz, 9H) ppm. ¹³C NMR (400 MHz, CDCl₃) δ=171.27, 164.12, 156.54, 155.90, 150.10, 136.95, 136.70, 132.19, 131.11, 131.06, 130.71, 130.01, 128.87, 77.16, 54.48, 40.82, 40.20, 39.19, 28.59, 14.56, 13.26, 11.99 ppm. LCMS (ESI): tᵣₑₜ=3.84 min. 543 m/z [M+H]⁺.

### N-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-2-(2,6-dioxopiperidin-3-yl)-1,3-dioxo-2,3,11,12-tetrahydro-1H-benzo[7,8][1,2]diazocino[3,4-f]isoindole-8-carboxamide (KR-157)

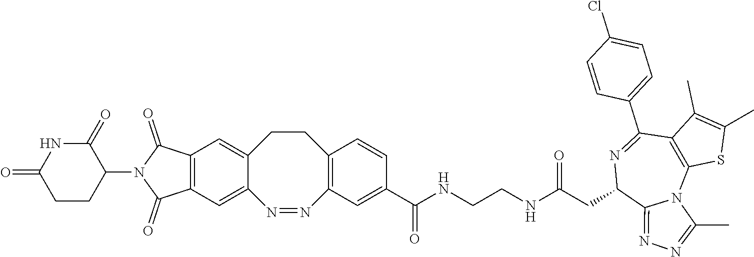 [SMILES: Cc1sc2c(c1C)C(c1ccc(Cl)cc1)=N[C@@H](CC(=O)NCCNC(=O)c1ccc3c(c1)/N=N\c1cc4c(cc1CC3)C(=O)N(C1CCC(=O)NC1=O)C4=O)c1nnc(C)n1-2]

KK-66 (8.4 mg, 19.00 μmol, 2.0 eq.) was dissolved in a CH₂Cl₂:TFA mixture (1:1; 2 mL). After 4 h the reaction was concentrated under reduced pressure. The mixture was triturated with MeOH and then dried under high vacuum for 48 h. 65 (4.1 mg, 9.50 μmol, 1.0 eq.) was dissolved in CH₂Cl₂:TFA (1:1; 1 mL each). After 4 h the reaction was concentrated under reduced pressure. The intermediate was purified by flash column chromatography (CH₂Cl₂/MeOH gradient, 0→50% MeOH). Both crude solids were dissolved in dry DMF (2 mL) and HATU (5.4 mg, 14.2 μmol, 1.5 eq.) was added at room temperature. i-Pr₂NEt (37.9 μmol, 4.0 eq., 7 μL) was added to the mixture and it was stirred for additional 12 h at room temperature. The reaction was diluted with EtOAc (5 mL), separated against H₂O/10% LiCl (1:1, 5 mL:5 mL), extracted with EtOAc (2×5 mL) and washed with 10% LiCl (2×5 mL) and brine (2×5 mL). The combined organic phase was dried over Na₂SO₄ and concentrated under reduced pressure. Purification of the resulting crude product by flash column chromatography (CH₂Cl₂/MeOH gradient, 0-20% MeOH) gave KR-157 (3.9 mg, 4.50 μmol, 48%) as a yellow solid. R_{f}=0.20 [CH₂Cl₂:MeOH, 19:1]. ¹H NMR (600 MHz, DMSO) δ=11.09 (s, 1H), 8.50 (s, J=5.1 Hz, 1H), 8.34 (d, J=4.5 Hz, 1H), 7.80 (s, 1H), 7.54 (d, J=7.8 Hz, 1H), 7.50 (d, J=6.0 Hz, 1H), 7.45 (t, J=7.7 Hz, 2H), 7.42-7.36 (m, 3H), 7.16 (d, J=7.9 Hz, 1H), 5.13-5.05 (m, 1H), 4.50 (t, J=6.8 Hz, 1H), 3.28-3.18 (m, 6H), 3.17-3.11 (m, 1H), 3.10-3.02 (m, 1H), 3.01-2.91 (m, 1H), 2.91-2.79 (m, 2H), 2.58 (s, 3H), 2.54 (d, J=7.7 Hz, 1H), 2.46-2.42 (m, 1H), 2.41 (d, J=3.1 Hz, 3H), 2.06-1.96 (m, 1H), 1.59 (d, J=14.6 Hz, 3H) ppm. ¹³C NMR (600 MHz, DMSO) δ=172.71, 169.93, 169.80, 166.32, 166.12, 165.11, 163.07, 159.67, 155.11, 154.31, 149.85, 136.74, 135.70, 135.19, 133.39, 132.27, 130.69, 130.52, 130.32, 130.15, 129.85, 129.56, 128.46, 126.46, 124.98, 117.84, 113.58, 53.79, 49.03, 39.52, 31.35, 31.29, 30.87, 30.11, 28.70, 22.09, 21.80, 16.74, 14.01, 12.68, 11.30 ppm. HRMS (APCI): calcd. for C₄₃H₃₈ClN₁₀O₆S⁺: 857.2380 m/z [M+H]⁺, found: 857.2394 m/z [M+H]⁺. LCMS (ESI): tᵣₑₜ=3.76 min. 857 m/z [M+H]⁺.

### tert-Butyl (2S,4R)-2-((3-aminobenzyl)carbamoyl)-4-hydroxypyrrolidine-1-carboxylate (KK-69)

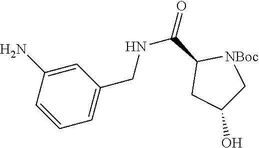 [SMILES: CC(C)(C)OC(=O)N1C[C@H](O)C[C@H]1C(=O)NCc1cccc(N)c1]

(2S,4R)-1-(tert-Butoxycarbonyl)-4-hydroxypyrrolidine-2-carboxylic acid (231.2 mg, 1.00 mmol, 1.0 eq.) and HATU (570.4 mg, 1.500 mmol, 1.5 eq.) were dissolved in dry DMF (5.0 mL) at RT. After 5 minutes of stirring, 3-(Aminomethyl)aniline (244.3 mg, 2.00 mmol, 2.0 eq.) and iPr₂NEt (3.00 mmol, 3.0 eq., 523 μL) were added to the mixture and stirred for further 12 h. The reaction was diluted with EtOAc (20 mL), separated against H₂O and extracted with EtOAc (3×20 mL). The combined organic phase was dried over Na₂SO₄ and concentrated under reduced pressure. KK-69 (328 mg, 0.978 mmol, 98%) was obtained as colorless oil. R_{f}=0.06 [CH₂Cl₂: MeOH, 4:1]. ¹H NMR (400 MHz, DMSO) δ=8.36-8.17 (m, 1H), 6.92 (t, J=7.6 Hz, 1H), 6.43 (s, 2H), 6.40 (d, J=6.9 Hz, 2H), 4.98 (s, J=1.8 Hz, 2H), 4.25-4.20 (m, 2H), 4.11-3.92 (m, 2H), 3.45-3.36 (m, 1H), 3.27 (d, J=11.1 Hz, 1H), 2.04 (dd, J=20.3, 10.0 Hz, 1H), 1.92-1.77 (m, 1H), 1.27 (d, J=13.3 Hz, 9H) ppm. ¹³C NMR (400 MHz, DMSO) δ=172.29, 171.97, 153.58, 148.57, 139.88, 128.66, 114.92, 113.01, 112.47, 78.49, 68.50, 67.87, 58.86, 54.78, 39.52, 27.96, 20.78, 14.10 ppm. HRMS (APCI): calcd. for C₁₇H₂₅N₃O₄⁺: 336.1845 m/z [M+H]⁺; found: 336.1879 m/z [M+H]⁺. LCMS (ESI): tᵣₑₜ=1.73 min. 236 m/z [M+2H-05H902]⁺.

### 4-((9-chloro-7-(2-fluoro-6-methoxyphenyl)-5H-benzo[c]pyrimido[4,5-e]azepin-2-yl)amino)-N-(4-(2-(4-((2-(2,6-dioxopiperidin-3-yl)-1-oxoisoindolin-4-yl)diazenyl)-2,6-dimethoxyphenoxy)acetamido)butyl)-2-methoxybenzamide (MR-MB-137)

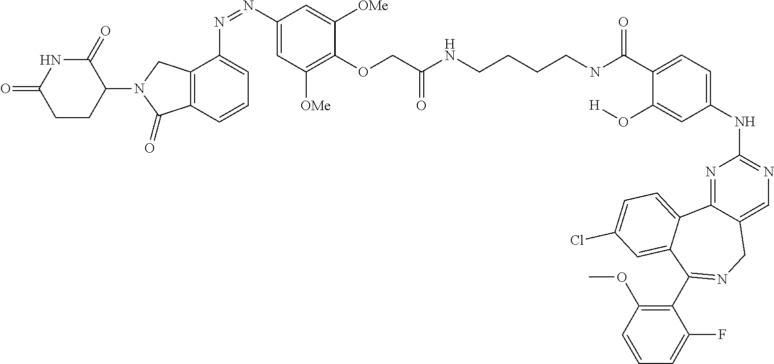 [SMILES: COc1cc(/N=N\c2cccc3c2CN(C2CCC(=O)NC2=O)C3=O)cc(OC)c1OCC(=O)NCCCCNC(=O)c1ccc(Nc2ncc3c(n2)-c2ccc(Cl)cc2C(c2c(F)cccc2OC)=NC3)cc1O]

Into a round bottom flask with Alisertib (7.8 mg, 0.015 mmol, 1 eq.) were added 4 (20.0 mg, 0.030 mmol, 2 eq.) and HATU (11.4 mg, 0.030 mmol, 2 eq.) under nitrogen atmosphere. The solids were dissolved in dry DMF (1 mL). After addition of i-Pr₂NEt (0.139 mmol, 9.2 eq., 0.024 mL) the reaction was stirred for 16 h at room temperature. The mixture was then diluted with EtOAc (20 mL), separated against 5% LiCl (25 mL), extracted with EtOAc (2×20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-137 (10.3 mg, 0.010 mmol, 65%) as a yellow solid. R_{f}=0.35 [CH₂Cl₂:MeOH, 15:1]. LCMS (ESI): tᵣₑₜ=4.26 min (Z). 1053 m/z [M+H]⁺. tᵣₑₜ=5.01 min (F). 1053 m/z [M+H]⁺.

### (1R,4R)-4-(3-chloro-2-fluorophenoxy)-N-(3-(2-(4-((2-(2,6-dioxopiperidin-3-yl)-1-oxoisoindolin-4-yl)diazenyl)-2,6-dimethoxyphenoxy)acetamido)propyl)-1-((6-(thiazol-2-ylamino)pyridin-2-yl)methyl)cyclohexane-1-carboxamide (MR-MB-200)

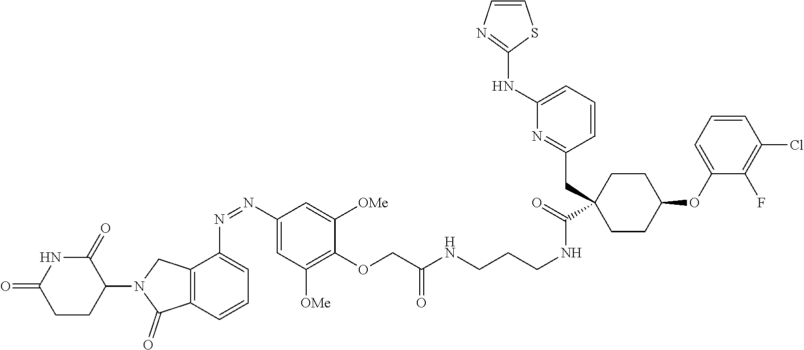 [SMILES: COc1cc(/N=N\c2cccc3c2CN(C2CCC(=O)NC2=O)C3=O)cc(OC)c1OCC(=O)NCCCNC(=O)[C@]1(Cc2cccc(Nc3nccs3)n2)CC[C@@H](Oc2cccc(Cl)c2F)CC1]

Into a round bottom flask with MK-5108 (7 mg, 0.015 mmol, 1 eq.) were added S9 (19.8 mg, 0.030 mmol, 2 eq.) and HATU (11.5 mg, 0.030 mmol, 2 eq.) under nitrogen atmosphere. The solids were dissolved in dry DMF (1 mL). After addition of i-Pr₂NEt (0.157 mmol, 10.4 eq., 0.027 mL) the reaction was stirred for 16 h at room temperature. The mixture was then diluted with EtOAc (20 mL), separated against 5% LiCl (25 mL), extracted with EtOAc (2×20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-200 (9.3 mg, 0.009 mmol, 63%) as a yellow solid. R_{f}=0.47 [CH₂Cl₂:MeOH, 9:1]. LCMS (ESI): tᵣₑₜ=4.04 min (Z). 982 m/z [M+H]⁺. tᵣₑₜ=4.20 min (E). 982 m/z [M+H]⁺.

### (1R,4R)-4-(3-chloro-2-fluorophenoxy)-N-(2-(2-(4-((2-(2,6-dioxopiperidin-3-yl)-1-oxoisoindolin-4-yl)diazenyl)-2,6-dimethoxyphenoxy)acetamido)ethyl)-1-((6-(thiazol-2-ylamino)pyridin-2-yl)methyl)cyclohexane-1-carboxamide (MR-MB-201)

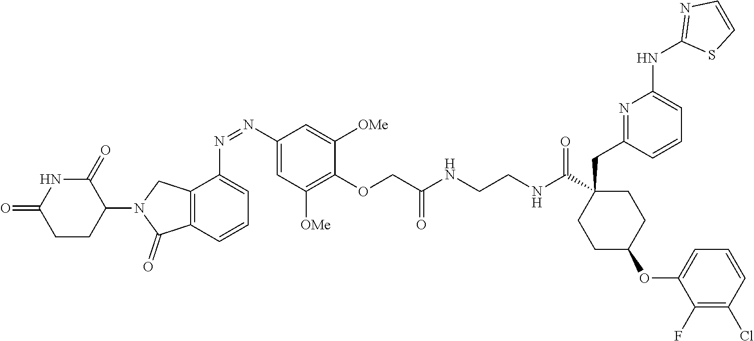 [SMILES: COc1cc(/N=N\c2cccc3c2CN(C2CCC(=O)NC2=O)C3=O)cc(OC)c1OCC(=O)NCCNC(=O)[C@]1(Cc2cccc(Nc3nccs3)n2)CC[C@@H](Oc2cccc(Cl)c2F)CC1]

Into a round bottom flask with MK-5108 (7 mg, 0.015 mmol, 1 eq.) were added S8 (19.4 mg, 0.030 mmol, 2 eq.) and HATU (11.5 mg, 0.030 mmol, 2 eq.) under nitrogen atmosphere. The solids were dissolved in dry DMF (1 mL). After addition of i-Pr₂NEt (0.157 mmol, 10.4 eq., 0.027 mL) the reaction was stirred for 16 h at room temperature. The mixture was then diluted with EtOAc (20 mL), separated against 5% LiCl (25 mL), extracted with EtOAc (2×20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-201 (11.5 mg, 0.012 mmol, 78%) as a yellow solid. R_{f}=0.44 [CH₂Cl₂:MeOH, 9:1]. LCMS (ESI): tᵣₑₜ=3.93 min (Z). 968 m/z [M+H]⁺. tᵣₑₜ=4.02 min (E). 968 m/z [M+H]⁺.

### (2R,3 S,4R,5 S)-3-(3-chloro-2-fluorophenyl)-4-(4-chloro-2-fluorophenyl)-4-cyano-N-(4-((4-(2-(4-((2-(2,6-dioxopiperidin-3-yl)-1-oxoisoindolin-4-yl)diazenyl)-2,6-dimethoxyphenoxy)acetamido)butyl)carbamoyl)-2-methoxyphenyl)-5-neopentylpyrrolidine-2-carboxamide (MR-MB-139)

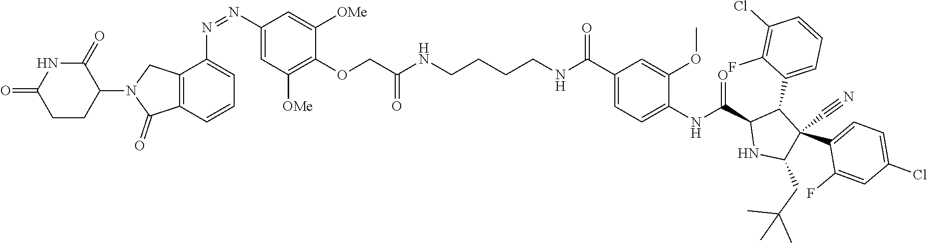 [SMILES: COc1cc(C(=O)NCCCCNC(=O)COc2c(OC)cc(/N=N\c3cccc4c3CN(C3CCC(=O)NC3=O)C4=O)cc2OC)ccc1NC(=O)[C@@H]1N[C@@H](CC(C)(C)C)[C@](C#N)(c2ccc(Cl)cc2F)[C@H]1c1cccc(Cl)c1F]

Into a round bottom flask with Idasanutlin (7.0 mg, 0.011 mmol, 1 eq.) were added 4 (16.0 mg, 0.024 mmol, 2.1 eq.) and HATU (9.1 mg, 0.024 mmol, 2.1 eq.) under nitrogen atmosphere. The solids were dissolved in dry DMF (1 mL). After addition of i-Pr₂NEt (0.087 mmol, 7.6 eq., 0.015 mL) the reaction was stirred for 16 h at room temperature. The mixture was then diluted with EtOAc (20 mL), separated against 5% LiCl (25 mL), extracted with EtOAc (2×20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-139 (11.5 mg, 0.010 mmol, 88%) as a yellow solid. R_{f}=0.38 [CH₂Cl₂:MeOH, 15:1]. LCMS (ESI): tᵣₑₜ=5.09 min (Z). 576 m/z [M+2H]²⁺. tᵣₑₜ=5.23 min (E). 576 m/z [M+2H]²⁺.

### N-(2-(2-((6-chlorohexyl)oxy)ethoxy)ethyl)-2-(4-((2-(2,6-dioxopiperidin-3-yl)-1-oxoisoindolin-4-yl)diazenyl)-2,6-dimethoxyphenoxy)acetamide (MR-MB-204)

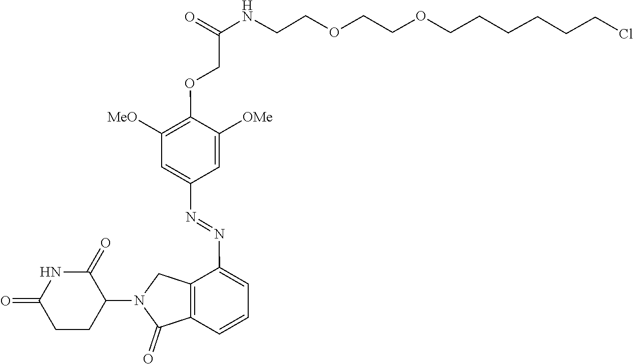 [SMILES: COc1cc(/N=N/c2cccc3c2CN(C2CCC(=O)NC2=O)C3=O)cc(OC)c1OCC(=O)NCCOCCOCCCCCCCl]

Into a round bottom flask with 3 (50 mg, 0.097 mmol, 1 eq.) and HATU (74.1 mg, 0.19 mmol, 2 eq.) in dry DMF (1 mL), was added under nitrogen atmosphere i-Pr₂NEt (0.39 mmol, 4 eq., 0.068 mL). After addition of 2-(2-((6-chlorohexyl)oxy)ethoxy)ethan-1-amine (65 mg, 0.29 mmol, 3 eq.) the reaction was stirred for 14 h at room temperature. The mixture was then diluted with EtOAc (20 mL), separated against 5% LiCl (25 mL), extracted with EtOAc (20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-204 (54.6 mg, 0.0179 mmol, 81%) as a yellow solid. R_{f}=0.22 [CH₂Cl₂:MeOH, 19:1]. LCMS (ESI): tᵣₑₜ=3.91 min (Z). 688 m/z [M+H]⁺. tᵣₑₜ=4.16 min (E). 688 m/z [M+H]⁺.

### N-(2-(2-((6-chlorohexyl)oxy)ethoxy)ethyl)-2-(4-((2-(2,6-dioxopiperidin-3-yl)-1-oxoisoindolin-4-yl)diazenyl)phenoxy)acetamide (MR-MB-205)

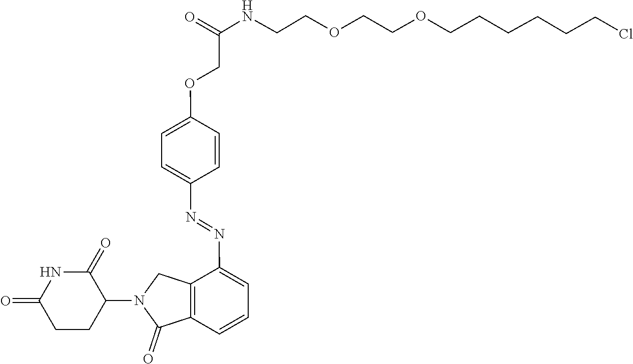 [SMILES: O=C(COc1ccc(/N=N/c2cccc3c2CN(C2CCC(=O)NC2=O)C3=O)cc1)NCCOCCOCCCCCCCl]

Into a round bottom flask with S14 (25 mg, 0.056 mmol, 1 eq.) and HATU (42.3 mg, 0.11 mmol, 2 eq.) in dry DMF (1 mL), was added under nitrogen atmosphere i-Pr₂NEt (0.22 mmol, 4 eq., 0.039 mL). After addition of 2-(2-((6-chlorohexyl)oxy)ethoxy)ethan-1-amine (37 mg, 0.17 mmol, 3 eq.) the reaction was stirred for 14 h at room temperature. The mixture was then diluted with EtOAc (20 mL), separated against 5% LiCl (25 mL), extracted with EtOAc (20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-205 (28.7 mg, 0.046 mmol, 81%) as a yellow solid. R_{f}=0.21 [CH₂Cl₂:MeOH, 19:1]. LCMS (ESI): tᵣₑₜ=3.72 min (Z). 628 m/z [M+H]⁺. tᵣₑₜ=4.03 min (E). 628 m/z [M+H]⁺.

### N-(3-*(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)propyl)-2-(4-((2-(2,6-dioxopiperidin-3-yl)-1-oxoisoindolin-4-yl)diazenyl)-2,6-dimethoxyphenoxy)acetamide (MR-MB-118)

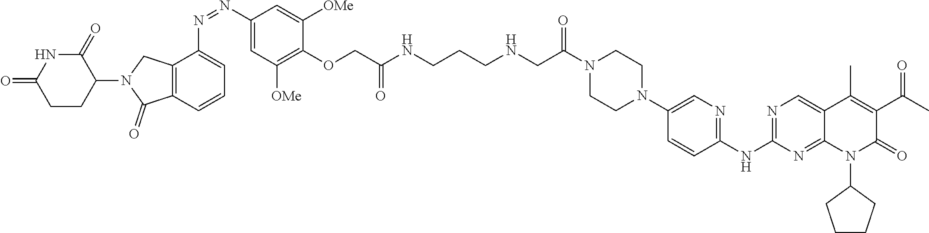 [SMILES: COc1cc(/N=N\c2cccc3c2CN(C2CCC(=O)NC2=O)C3=O)cc(OC)c1OCC(=O)NCCCNCC(=O)N1CCN(c2ccc(Nc3ncc4c(C)c(C(C)=O)c(=O)n(C5CCCC5)c4n3)nc2)CC1]

Into a round bottom flask with 6-acetyl-2-((5-(4-(2-chloroacetyl)piperazin-1-yl)pyridin-2-yl)amino)-8-cyclopentyl-5-methylpyrido[2,3-d]pyrimidin-7(8H)-one (14.6 mg, 0.028 mmol, 1 eq.) were added S9 (27.3 mg, 0.042 mmol, 1.5 eq.) and Na₂CO₃ (5.9 mg, 0.056 mmol, 2 eq.) under nitrogen atmosphere. The solids were dissolved in dry DMF (6.5 mL). The reaction was stirred for 24 h at 80° C. The reaction mixture was then diluted with a 5:1 CH₂Cl₂:iPrOH mixture (20 mL), separated against 5% LiCl (25 mL), washed with 10% LiCl (25 mL) and brine (2×25 mL). The organic phase was dried over Na₂SO₄ and concentrated under reduced pressure. Purification of the resulting crude product by preparative TLC (CH₂Cl₂:MeOH:iPrOH, 9:1:0.5) gave MR-MB-118 (8.1 mg, 0.008 mmol, 28%) as a yellow solid. LCMS (ESI): tᵣₑₜ=2.90 min (E). 514 m/z [M+2H]²⁺.

### N-(4-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)butyl)-2-(4-((2-(2,6-dioxopiperidin-3-yl)-1-oxoisoindolin-4-yl)diazenyl)-2,6-dimethoxyphenoxy)acetamide (MR-MB-125)

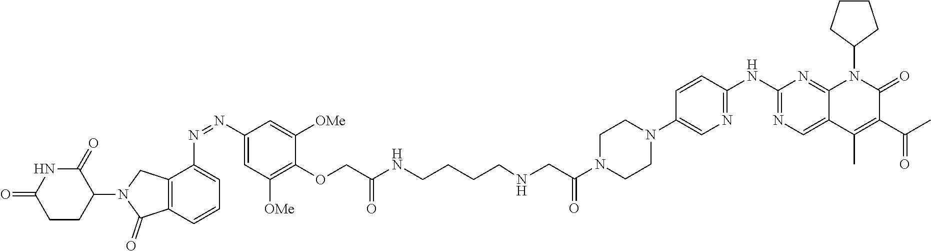 [SMILES: COc1cc(/N=N\c2cccc3c2CN(C2CCC(=O)NC2=O)C3=O)cc(OC)c1OCC(=O)NCCCCNCC(=O)N1CCN(c2ccc(Nc3ncc4c(C)c(C(C)=O)c(=O)n(C5CCCC5)c4n3)nc2)CC1]

Into a round bottom flask with 6-acetyl-2-((5-(4-(2-chloroacetyl)piperazin-1-yl)pyridin-2-yl)amino)-8-cyclopentyl-5-methylpyrido[2,3-d]pyrimidin-7(8H)-one (18.8 mg, 0.036 mmol, 1 eq.) were added 4 (35.9 mg, 0.054 mmol, 1.5 eq.) and Na₂CO₃ (7.6 mg, 0.072 mmol, 2 eq.) under nitrogen atmosphere. The solids were dissolved in dry DMF (3 mL). The reaction was stirred for 20 h at 50° C. The reaction mixture was then diluted with a 5:1 CH₂Cl₂:iPrOH mixture (20 mL), separated against 5% LiCl (25 mL), washed with 10% LiCl (25 mL) and brine (2×25 mL). The organic phase was dried over Na₂SO₄ and concentrated under reduced pressure. Purification of the resulting crude product by preparative TLC (CH₂Cl₂:MeOH:iPrOH, 9:1:0.5) gave MR-MB-125 (12.0 mg, 0.012 mmol, 32%) as a yellow solid. LCMS (ESI): tᵣₑₜ=2.98 min (E). 521 m/z [M+2H]²⁺.

### N-(5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)pentyl)-2-(4-((2-(2,6-dioxopiperidin-3-yl)-1-oxoisoindolin-4-yl)diazenyl)-2,6-dimethoxyphenoxy)acetamide (MR-MB-104)

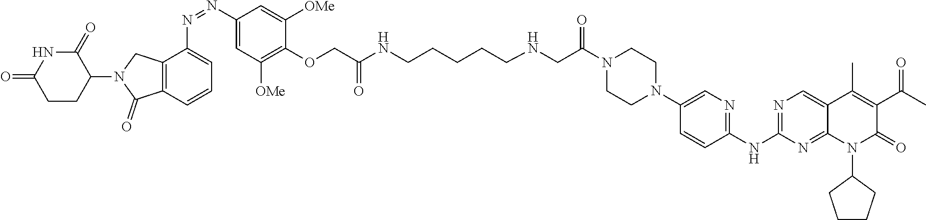 [SMILES: COc1cc(/N=N\c2cccc3c2CN(C2CCC(=O)NC2=O)C3=O)cc(OC)c1OCC(=O)NCCCCCNCC(=O)N1CCN(c2ccc(Nc3ncc4c(C)c(C(C)=O)c(=O)n(C5CCCC5)c4n3)nc2)CC1]

Into a round bottom flask with 6-acetyl-2-((5-(4-(2-chloroacetyl)piperazin-1-yl)pyridin-2-yl)amino)-8-cyclopentyl-5-methylpyrido[2,3-d]pyrimidin-7(8H)-one (7.0 mg, 0.013 mmol, 1 eq.) were added S10 (13.6 mg, 0.020 mmol, 1.5 eq.) and Na₂CO₃ (2.8 mg, 0.027 mmol, 2 eq.) under nitrogen atmosphere. The solids were dissolved in dry acetonitrile (3 mL). The reaction was stirred for 24 h at room temperature. The reaction mixture was then diluted with a 5:1 CH₂Cl₂:iPrOH mixture (20 mL), separated against 5% LiCl (25 mL), washed with 10% LiCl (25 mL) and brine (2×25 mL). The organic phase was dried over Na₂SO₄ and concentrated under reduced pressure. Purification of the resulting crude product by preparative TLC (CH₂Cl₂:MeOH:iPrOH, 9:1:0.5) gave MR-MB-104 (2.7 mg, 0.003 mmol, 19%) as a yellow solid. LCMS (ESI): tᵣₑₜ=3.02 min (E). 528 m/z [M+2E1]²⁺.

### 3-(4-((4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)phenyl)diazenyl)-1-oxoisoindolin-2-yl)piperidine-2,6-dione (MR-MB-142)

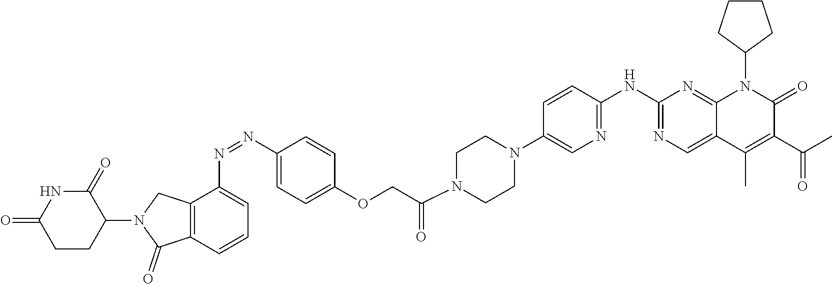 [SMILES: CC(=O)c1c(C)c2cnc(Nc3ccc(N4CCN(C(=O)COc5ccc(/N=N\c6cccc7c6CN(C6CCC(=O)NC6=O)C7=O)cc5)CC4)cn3)nc2n(C2CCCC2)c1=O]

Into a round bottom flask under nitrogen atmosphere with S14 (23.1 mg, 0.043 mmol, 1 eq.), palbociclib (38.5 mg, 0.086 mmol, 2 eq.) and HATU (29.4 mg, 0.077 mmol, 1.8 eq.) was added dry DMF (1 mL). After addition of i-Pr₂NEt (0.215 mmol, 5 eq., 0.037 mL) the reaction was stirred for 17 h at room temperature. The mixture was then diluted with EtOAc (20 mL), separated against 5% LiCl (25 mL), extracted with EtOAc (20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-142 (36.0 mg, 0.042 mmol, 98%) as a yellow solid. R_{f}=0.24 [CH₂Cl₂:MeOH, 20:1]. LCMS (ESI): tᵣₑₜ=3.58 min (E). 852 m/z [M+H]⁺.

### 3-(4-((4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)-3,5-dimethoxyphenyl)diazenyl)-1-oxoisoindolin-2-yl)piperidine-2,6-di one (MR-MB-145)

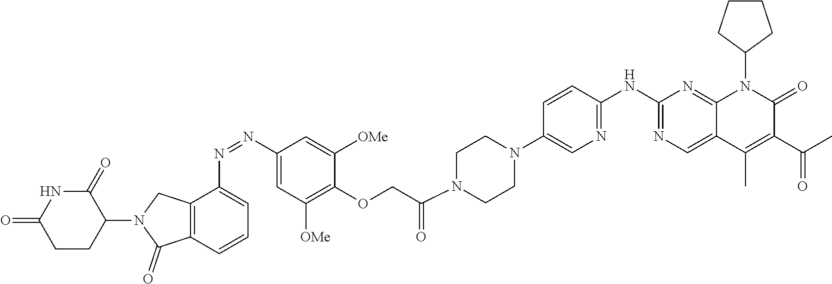 [SMILES: COc1cc(/N=N\c2cccc3c2CN(C2CCC(=O)NC2=O)C3=O)cc(OC)c1OCC(=O)N1CCN(c2ccc(Nc3ncc4c(C)c(C(C)=O)c(=O)n(C5CCCC5)c4n3)nc2)CC1]

Into a round bottom flask under nitrogen atmosphere with 3 (20.0 mg, 0.039 mmol, 1 eq.), palbociclib (35.3 mg, 0.079 mmol, 2 eq.) and HATU (27.0 mg, 0.071 mmol, 1.8 eq.) was added dry DMF (1 mL). After addition of i-Pr₂NEt (0.197 mmol, 5 eq., 0.034 mL) the reaction was stirred for 17 h at room temperature. The reaction mixture was then diluted with a 5:1 CH₂Cl₂:iPrOH mixture (20 mL), separated against 5% LiCl (25 mL), washed with 10% LiCl (25 mL) and brine (2×25 mL). The organic phase was dried over Na₂SO₄ and concentrated under reduced pressure. Purification of the resulting crude product by flash column chromatography (CH₂Cl₂/MeOH gradient, 0→20% MeOH) gave MR-MB-145 (33.0 mg, 0.042 mmol, 92%) as a yellow solid. LCMS (ESI): tᵣₑₜ=3.63 min (E). 912 m/z [M+H]⁺.

### 7-cyclopentyl-2-((5-(4-(2-(4-((2-(2,6-dioxopiperidin-3-yl)-1-oxoisoindolin-4-yl)diazenyl)-2,6-dimethoxyphenoxy)acetyl)piperazin-1-yl)pyridin-2-yl)amino)-N,N-dimethyl-7H-pyrrolo[2,3-d]pyrimidine-6-carboxamide (MR-MB-148)

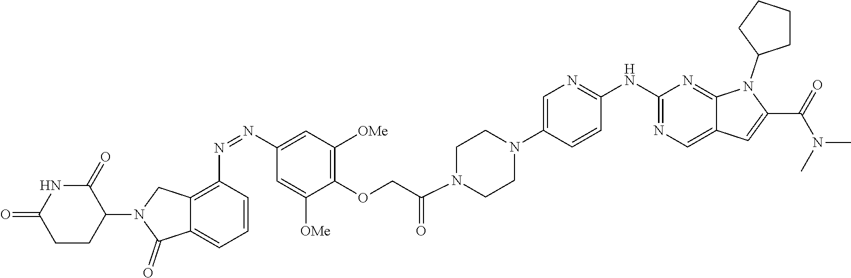 [SMILES: COc1cc(/N=N\c2cccc3c2CN(C2CCC(=O)NC2=O)C3=O)cc(OC)c1OCC(=O)N1CCN(c2ccc(Nc3ncc4cc(C(=O)N(C)C)n(C5CCCC5)c4n3)nc2)CC1]

Into a round bottom flask under nitrogen atmosphere with 3 (15.0 mg, 0.030 mmol, 1 eq.), ribociclib (12.8 mg, 0.03 mmol, 1 eq.) and HATU (13.5 mg, 0.035 mmol, 1.2 eq.) was added dry DMF (1 mL). After addition of i-Pr₂NEt (0.148 mmol, 5 eq., 0.026 mL) the reaction was stirred for 17 h at room temperature. The reaction mixture was then diluted with a 5:1 CH₂Cl₂:iPrOH mixture (20 mL), separated against 5% LiCl (25 mL), washed with 10% LiCl (25 mL) and brine (2×25 mL). The organic phase was dried over Na₂SO₄ and concentrated under reduced pressure. Purification of the resulting crude product by flash column chromatography (CH₂Cl₂/MeOH gradient, 0→20% MeOH) gave MR-MB-148 (20.3 mg, 0.023 mmol, 77%) as a yellow solid. R_{f}=0.24 [CH₂Cl₂:MeOH, 20:1]. LCMS (ESI): tᵣₑₜ=3.13 min (Z). 899 m/z [M+H]⁺. tᵣₑₜ=3.36 min (E). 899 m/z [M+H]⁺.

### N-(5-(((5-(tert-butyl)oxazol-2-yl)methyl)thio)thiazol-2-yl)-1-(2-(4-((2-(2,6-dioxopiperidin-3-yl)-1-oxoisoindolin-4-yl)diazenyl)-2,6-dimethoxyphenoxy)acetyl)piperidine-4-carboxamide (MR-MB-156)

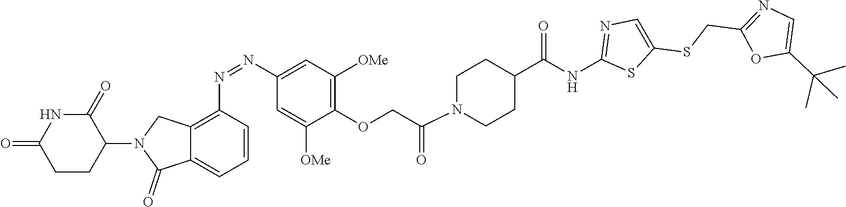 [SMILES: COc1cc(/N=N\c2cccc3c2CN(C2CCC(=O)NC2=O)C3=O)cc(OC)c1OCC(=O)N1CCC(C(=O)Nc2ncc(SCc3ncc(C(C)(C)C)o3)s2)CC1]

Into a round bottom flask with 3 (15 mg, 0.030 mmol, 1 eq.) and HATU (13.5 mg, 0.035 mmol, 1.2 eq.) in dry DMF (1 mL), was added under nitrogen atmosphere i-Pr₂NEt (0.148 mmol, 5 eq., 0.026 mL). After addition of BMS-387032 (11.2 mg, 0.030 mmol, 1 eq.) the reaction was stirred for 16 h at room temperature. The mixture was then diluted with EtOAc (20 mL), separated against 5% LiCl (25 mL), extracted with EtOAc (20 mL), washed with 10% LiCl (25 mL) and brine (2×25 mL). The combined organic phases were dried over Na₂SO₄ and concentrated under reduced pressure. Purification of the resulting crude product by flash column chromatography (CH₂Cl₂/MeOH gradient, 0→20% MeOH) gave MR-MB-156 (18.3 mg, 0.022 mmol, 73%) as a yellow solid. R_{f}=0.26 [CH₂Cl₂:MeOH, 20:1]. LCMS (ESI): tᵣₑₜ=3.98 min (E). 845 m/z [M+H]⁺.

Cell culture. The human acute lymphoblastic leukemia RS4;11 (ATCC® CRL-1873™) cell line was purchased from the American Type Culture Collection and cultured in RPMI1640 medium (Gibco) with 10% fetal bovine serum (FBS) and 1% penicillin/streptomycin (PS) in a humidified incubator at 37° C. with 5% CO₂ in air. Cells were divided every 2-3 days and the concentration was maintained between 1×10⁵ and 1×10⁶ cells/mL. For the experiments compounds were serially diluted in RPMI160 without the dye phenol red (Gibco) to reduce the influence of the dye by absorption. Azobenzene stocks and dilutions were strictly kept in the dark and prepared under red light conditions.

Colorimetric MTT Assays. The activity of dehydrogenase enzymes in metabolically active cells, as a quantitative measurement for cytotoxicity and proliferation, was determined by colorimetric measurement of the reduction of [3-(4,5-dimethylthiazol-2-yl)-5-(3-carboxymethoxyphenyl)-2-(4-sulfophenyl)-2H-tetrazolium (MTS) to formazan. The absorbance of formazan at 490 nm was measured on a FLUOstar Omega microplate reader (BMG Labtech).

Cells were treated with different concentrations, ranging from 10 μM to 1 nM, of our compounds and incubated on a 96-well plate for 72 h. They were placed in light-proof boxes and exposed to the lighting conditions specified in the experiment for 72 h. Next, 10 μL of Promega CellTiter 96® AQᵤₑₒᵤₛ One Solution Reagent was added to each well and incubated for further 4-7 hours. The absorbance at 490 nm was then recorded with a 96-well plate reader.

Western Immunoblot Analysis. Rabbit monoclonal Antibodies for BRD4 (#13440), β-Actin (#4970) c-Myc (#5605) and Anti-rabbit IgG, HRP-linked Antibody (#7074) were purchased from Cell Signalling Technologies.

Cells were incubated with different concentrations of selected compounds. They were placed in a light-proof box and preirradiated for 1 min at 390 nm followed by 100 ms pulses every 10 s or were kept in the dark for 4 h.

Cultured cells were collected by centrifugation and washed twice with cold PBS. The supernatant was removed and each cell pellet was lysed in 100 μL NP40-Cell-Lysis Buffer+0.1% SDS+1 mM PMSF+ protease inhibitor cocktail (Sigma Aldrich) for 30 min on ice, with vortexing at 10-minute intervals. Afterwards, cells were centrifuged at 13.000 rpm for 10 min at 4° C. Samples were stored on ice and the supernatant was collected for protein concentration determination using a Thermofisher Scientific Pierce™ Coomassie Plus (Bradford) Assay Kit.

The samples were diluted each with 5 μL Laemmli sample buffer Invitrogen (NuPAGE LDS Sample Buffer 4×) and 1 μL DTT, heated at 95° C. for 5 min. Respectively 20 μL of each sample was loaded in a lane of a Bio-Rad Mini-PROTEAN TGX precast Gel, 4-20%, used with tris-glycine SDS Running buffer. The gel ran 5 min at 50 V, then 35 min at 140 V. Next step was the electrotransfer to a polyvinylidene difluoride (PVDF) membrane which was carried out using an iBlot™ 2 Dry Blotting System set to the standard program. After the transfer, the membrane was washed with 25 mL TB ST for 5 min and subsequent incubated in 25 mL of blocking buffer (1×TBST+5% nonfat dry milk, Blotto, Santa Cruz Biotechnology) for 1 h at room temperature. The membrane was washed three times for 5 min each with 15 mL of TBST and incubated in a 10 mL antibody dilution buffer with gentle agitation at 4° C. overnight (Actin: 1:1000; c-MYC: 1:1000; BRD4: 1:1000). The next day, the membrane was washed again three times for 5 min each with 15 mL TBST, followed by incubation with anti-rabbit, HRP-linked antibody (1:2000) in 10 mL blocking buffer with gentle agitation for 1 h at room temperature. Afterwards, the membrane was washed three times for 5 min each with 15 mL TBST and then incubated in 10 mL of Clarity Max Western ECL Substrate solution for 5 min. Chemoluminescence imaging was performed on a Bio-Rad ChemiDoc™ Imaging System.

LED illumination. For illumination of the cells we used the cell disco system as previously described in the literature^{[25]}. 390 nm 5 mm LEDs were purchased from Roithner Lasertechnik. Cells were preirradiated for 1 min at 390 nm, followed by 100 ms pulses every 10 s in 96- or 6-well plates.

Although the present disclosure has been described with respect to one or more particular embodiment(s) and/or example(s), it will be understood that other embodiments and/or examples of the present disclosure may be made without departing from the scope of the present disclosure.

1. A compound having the following structure:
A-PS-L-B, A-L-PS-B, PS-A-L-B, PS-A-L-B-PS, A-PS-L-PS-B, A-L-PS-L-B, PS-A-L-PS-B, A-PS-L-B-PS, or A′-L′-B′,
wherein A is an E3 ligase ligand, PS is a photoswitchable group, L is optional and is a linker, B is a ligand for a target protein, A′ is an E3 ligase ligand optionally comprising a photoswitchable group, L′ is an optional linker optionally comprising a photoswitchable group, and B′ is a ligand for a target protein optionally comprising a photoswitchable group.

2. A compound of claim 1, wherein the photoswitchable group is chosen from: 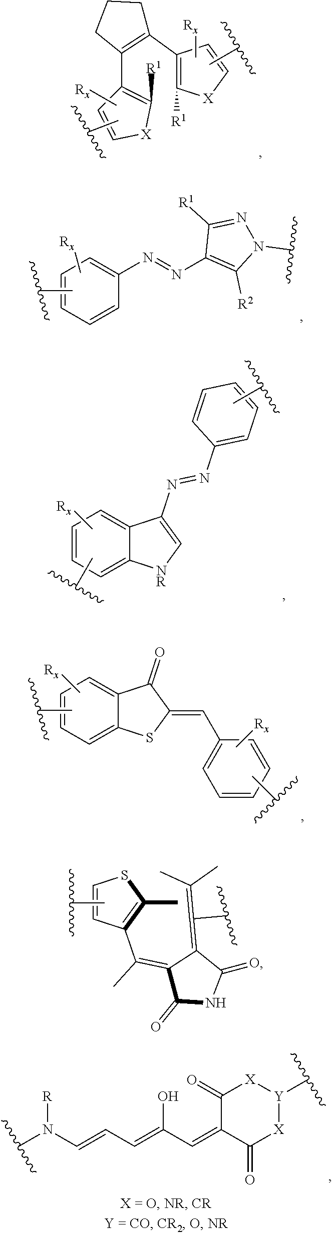 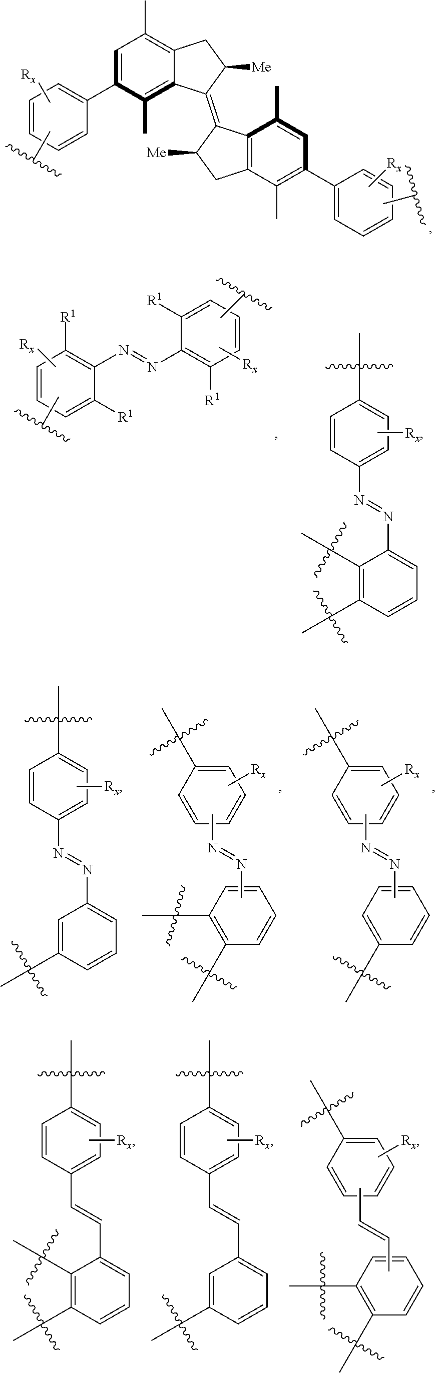 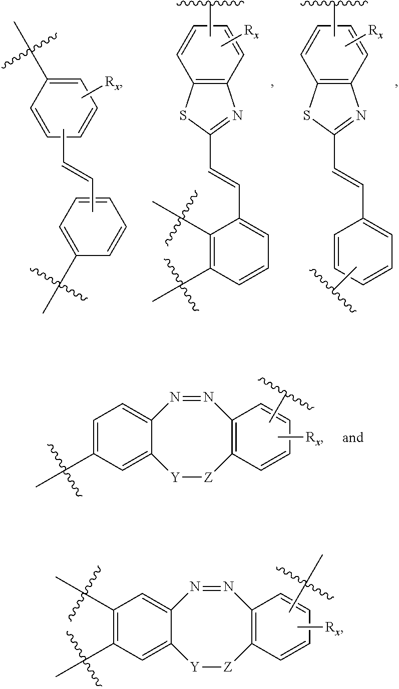
wherein R¹ is F, Cl, Me, or OMe; R is H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-Alkyl, CC-aryl, CO₂H, B(OH)₂, B(O-alkyl)₂, and B(O-aryl)₂; x is 0, 1, 2, 3, 4, or 5; X is methylene, C═O, or C═S, and Y is methylene, O, or S; and Z is methylene, O, or S.

3. A compound of claim 1, wherein the linker is chosen from: 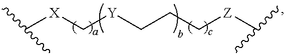
wherein Y is methylene or O, X and Z are independently methylene, O, NH, S, 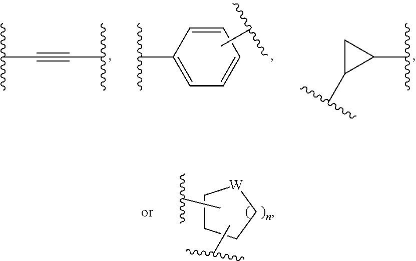
where W is methylene, NH, O, or S and n 0, 1, 2, 3, or 4; and a is 0-10, b is 0-10, and c is 0-10; 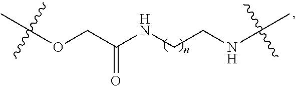
wherein n is 2, 3, 4, or 5;
groups formed from polyethylene glycol groups; alkyl linkers; and peptide-based linkers.

4. A compound of claim 1, wherein the E3 ligase ligand is a ligand for VHL, CRBN, RNF114, MDM2, DCAF15, DCAF16, Keap1, SCF, or a combination thereof.

5. A compound of claim 1, wherein E3 ligase ligand is chosen from: 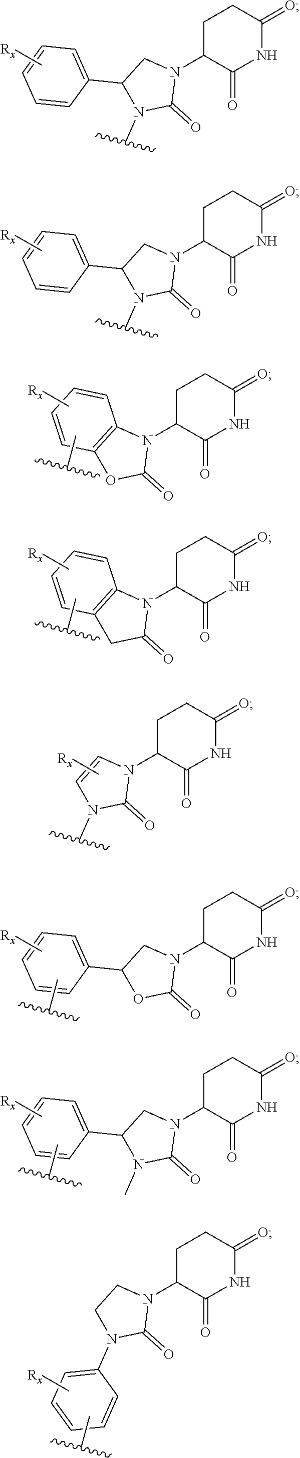 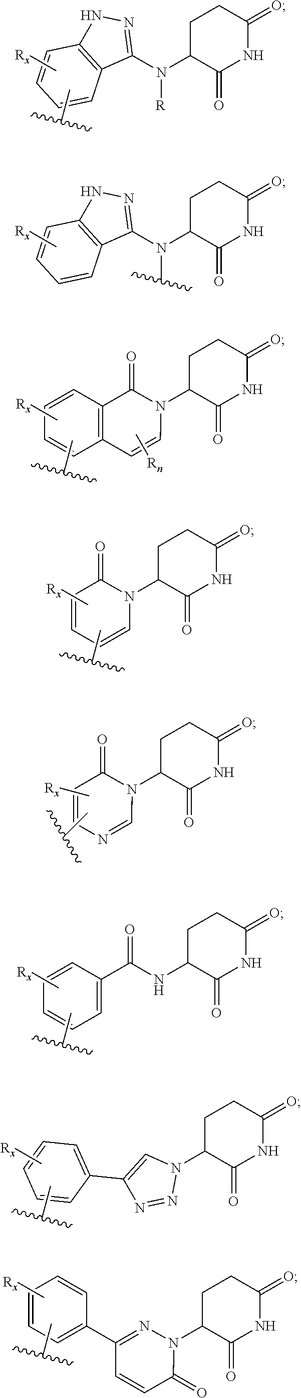 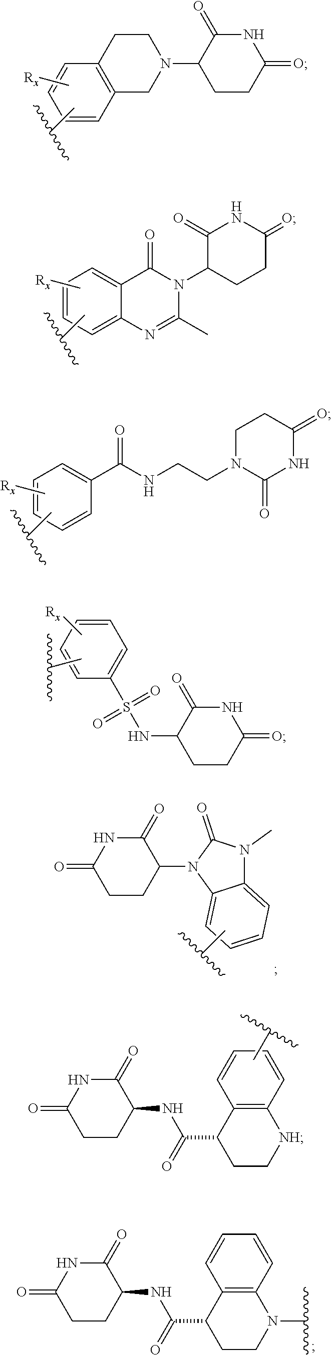 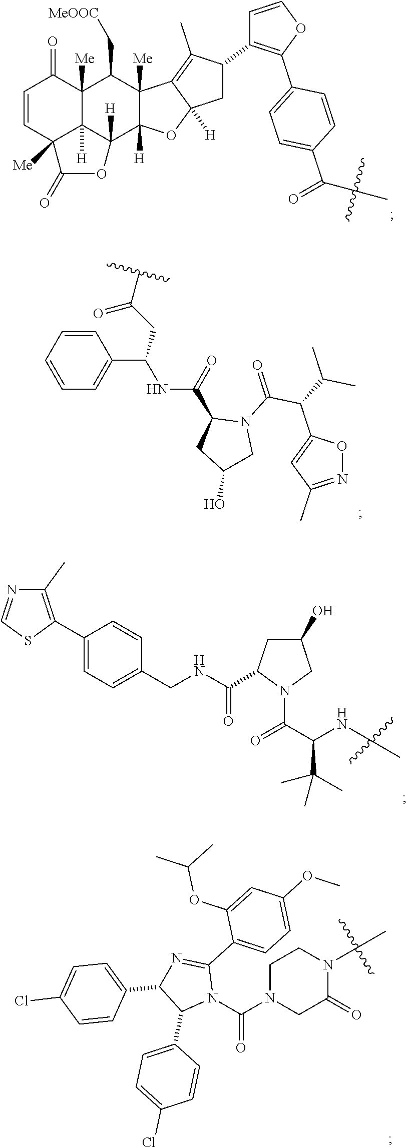 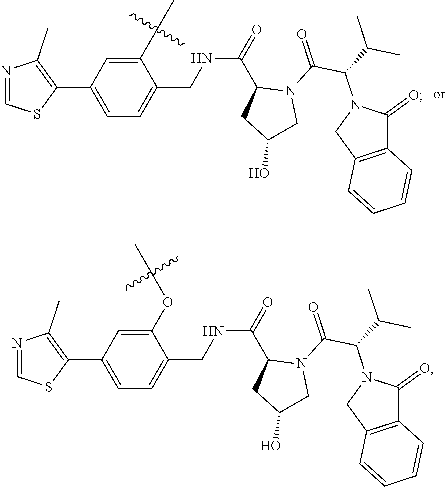
wherein R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂, a photoswitchable group, H2N-(D-R)₈-PIYALA- (SEQ ID NO:1), GGGGGGRAEDS*GNES*EGE-COOH (SEQ ID NO:2), wherein * is a phosphorylated serine, and GGGGGGDRIIDS*GLDS*M-COOH (SEQ ID NO:3), wherein * is a phosphorylated serine, and x is 0, 1, 2, 3, 4, or 5.

6. A compound of claim 1, wherein the ligand for a target protein is chosen from: 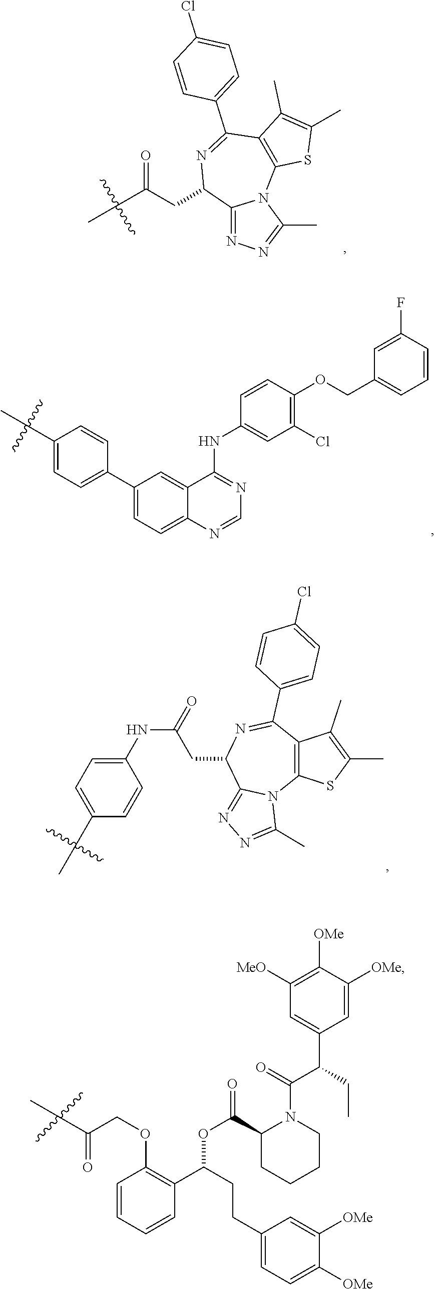 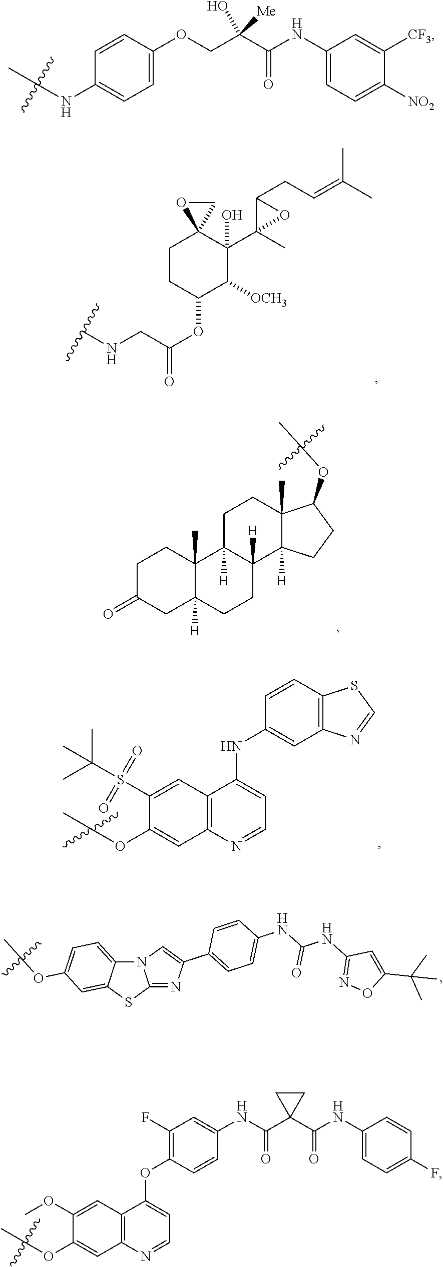 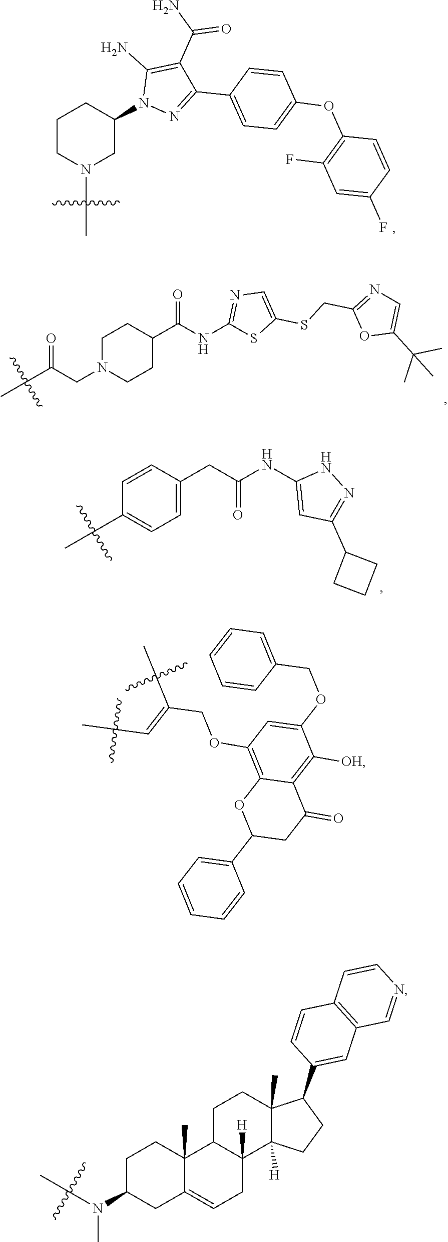 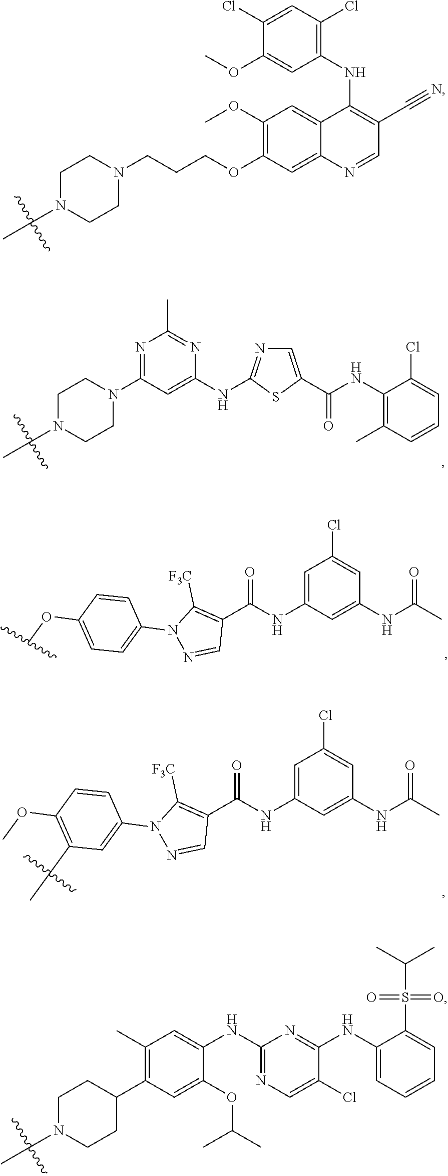 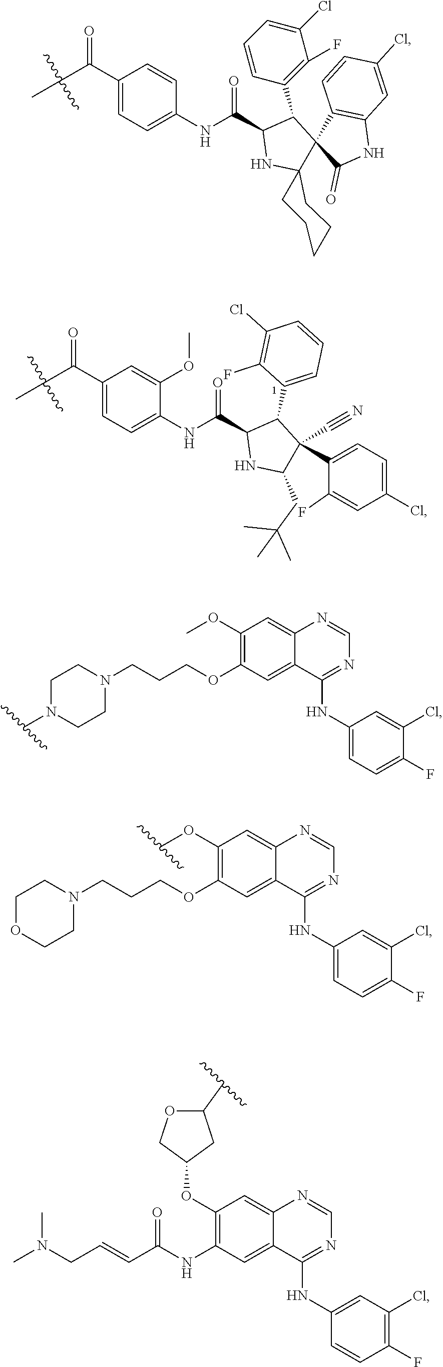 [SMILES: *C(=O)c1ccc(NC(=O)[C@@H]2NC3(CCCCC3)[C@]3(C(=O)Nc4cc(Cl)ccc43)[C@H]2c2cccc(Cl)c2F)cc1 ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *N1CCN(CCCOc2cc3c(Nc4ccc(F)c(Cl)c4)ncnc3cc2OC)CC1 ; *Oc1cc2ncnc(Nc3ccc(F)c(Cl)c3)c2cc1OCCCN1CCOCC1 ; *C1C[C@H](Oc2cc3ncnc(Nc4ccc(F)c(Cl)c4)c3cc2NC(=O)/C=C/CN(C)C)CO1] 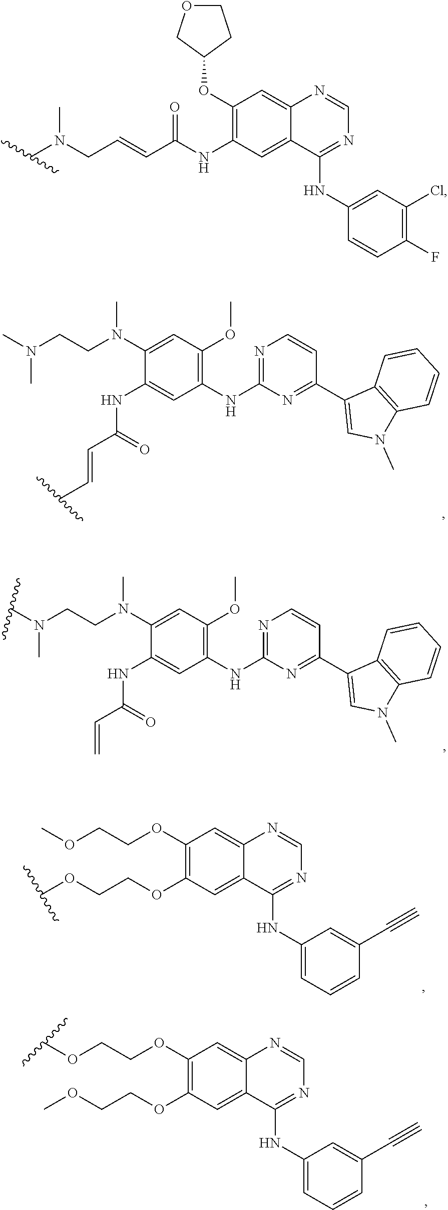 [SMILES: *N(C)C/C=C/C(=O)Nc1cc2c(Nc3ccc(F)c(Cl)c3)ncnc2cc1O[C@H]1CCOC1 ; */C=C/C(=O)Nc1cc(Nc2nccc(-c3cn(C)c4ccccc34)n2)c(OC)cc1N(C)CCN(C)C ; *N(C)CCN(C)c1cc(OC)c(Nc2nccc(-c3cn(C)c4ccccc34)n2)cc1NC(=O)C=C ; *OCCOc1cc2c(Nc3cccc(C#C)c3)ncnc2cc1OCCOC ; *OCCOc1cc2ncnc(Nc3cccc(C#C)c3)c2cc1OCCOC] 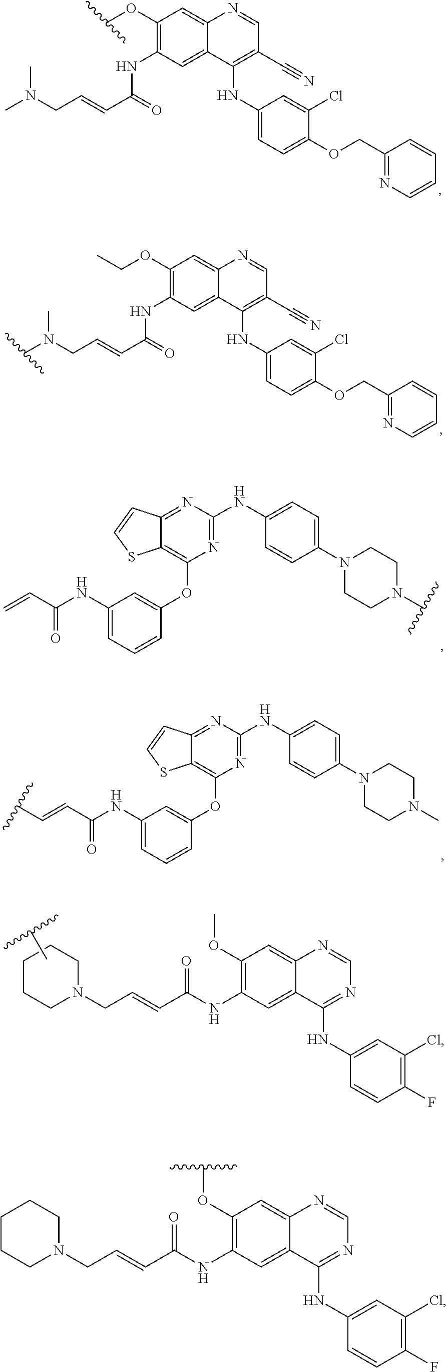 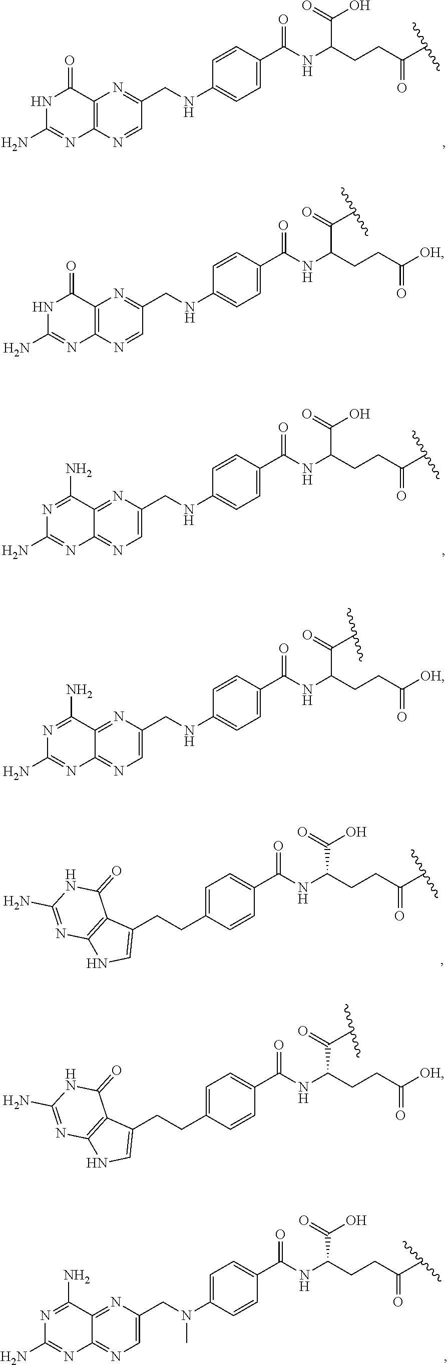 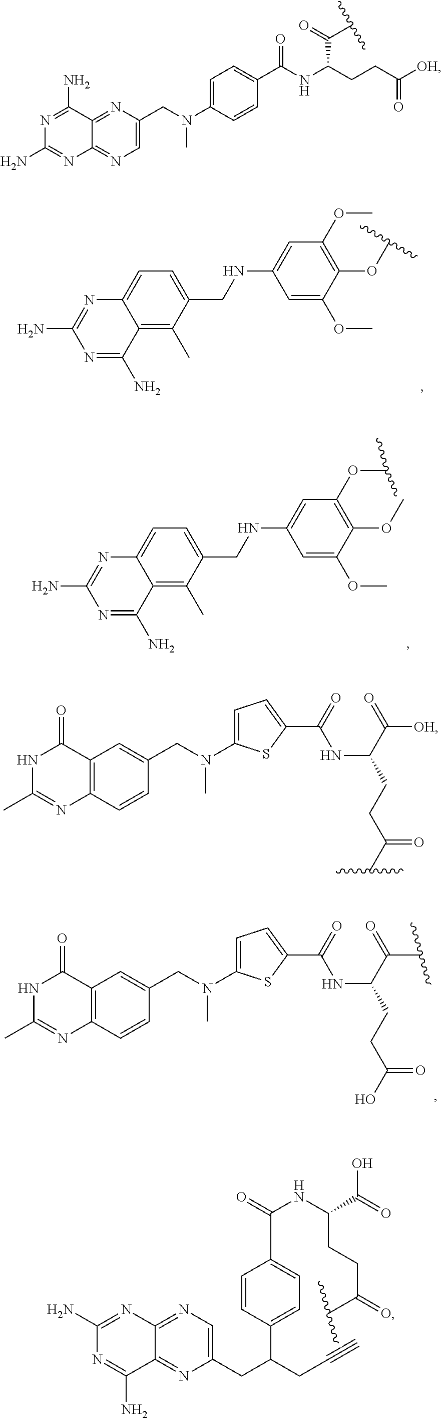 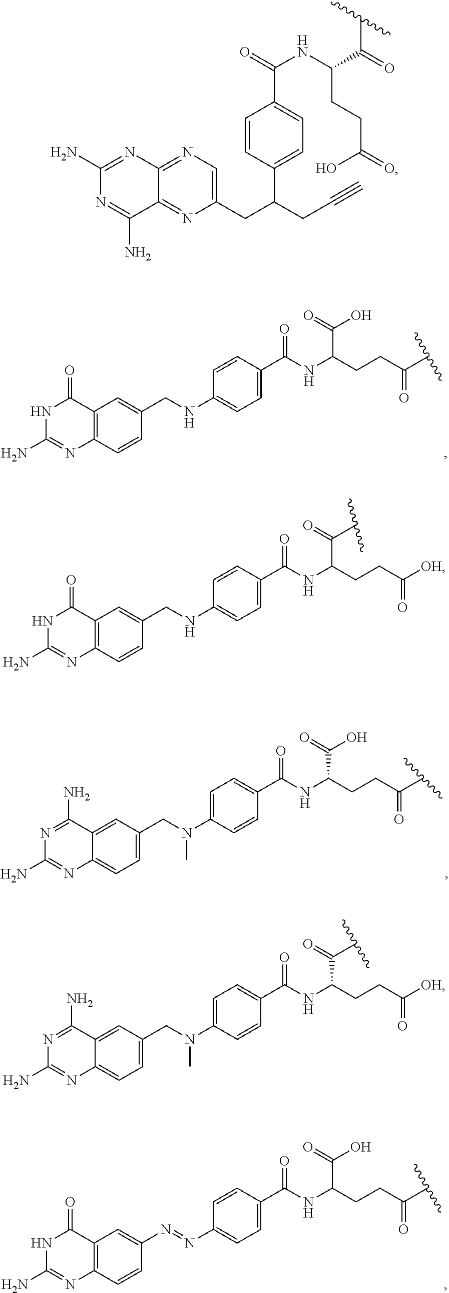 [SMILES: *C(=O)[C@H](CCC(=O)O)NC(=O)c1ccc(C(CC#C)Cc2cnc3nc(N)nc(N)c3n2)cc1 ; *C(=O)CCC(NC(=O)c1ccc(NCc2ccc3nc(N)[nH]c(=O)c3c2)cc1)C(=O)O ; *C(=O)C(CCC(=O)O)NC(=O)c1ccc(NCc2ccc3nc(N)[nH]c(=O)c3c2)cc1 ; *C(=O)CC[C@H](NC(=O)c1ccc(N(C)Cc2ccc3nc(N)nc(N)c3c2)cc1)C(=O)O ; *C(=O)[C@H](CCC(=O)O)NC(=O)c1ccc(N(C)Cc2ccc3nc(N)nc(N)c3c2)cc1 ; *C(=O)CCC(NC(=O)c1ccc(/N=N/c2ccc3nc(N)[nH]c(=O)c3c2)cc1)C(=O)O] 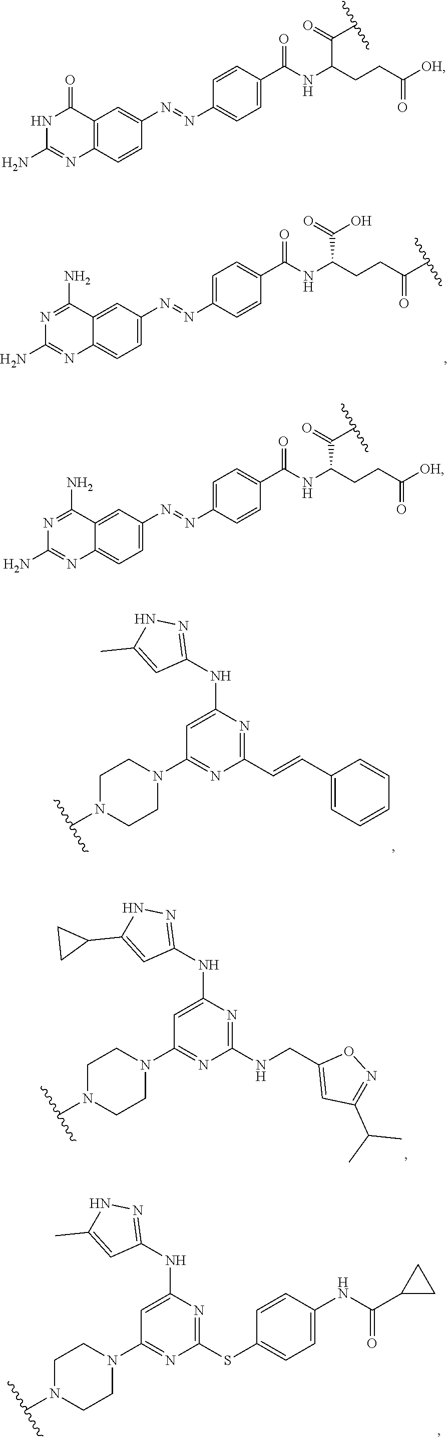 [SMILES: *C(=O)C(CCC(=O)O)NC(=O)c1ccc(/N=N/c2ccc3nc(N)[nH]c(=O)c3c2)cc1 ; *C(=O)CC[C@H](NC(=O)c1ccc(/N=N/c2ccc3nc(N)nc(N)c3c2)cc1)C(=O)O ; *C(=O)[C@H](CCC(=O)O)NC(=O)c1ccc(/N=N/c2ccc3nc(N)nc(N)c3c2)cc1 ; *N1CCN(c2cc(Nc3cc(C)[nH]n3)nc(/C=C/c3ccccc3)n2)CC1 ; *N1CCN(c2cc(Nc3cc(C4CC4)[nH]n3)nc(NCc3cc(C(C)C)no3)n2)CC1 ; *N1CCN(c2cc(Nc3cc(C)[nH]n3)nc(Sc3ccc(NC(=O)C4CC4)cc3)n2)CC1] 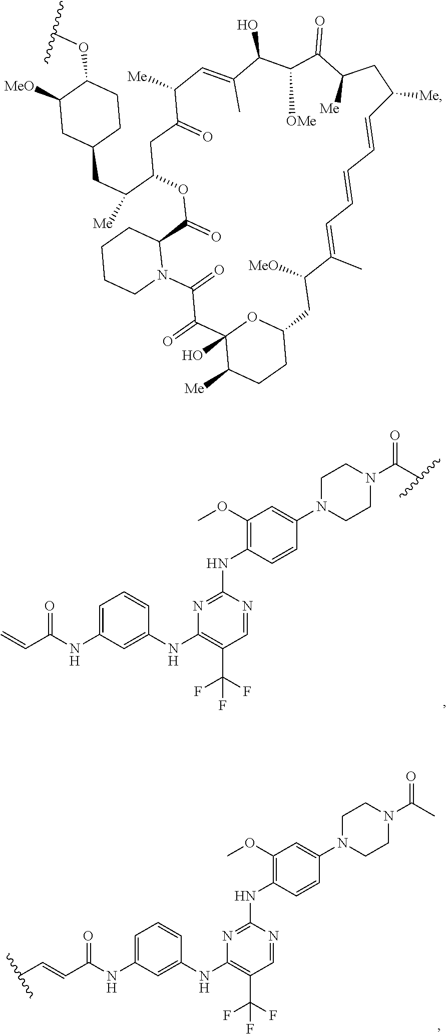 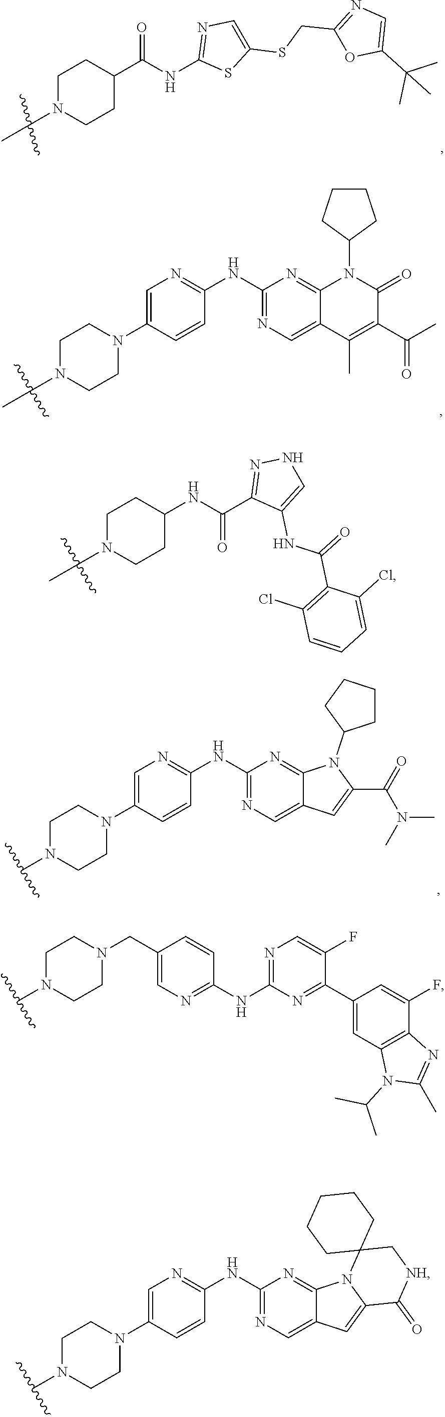 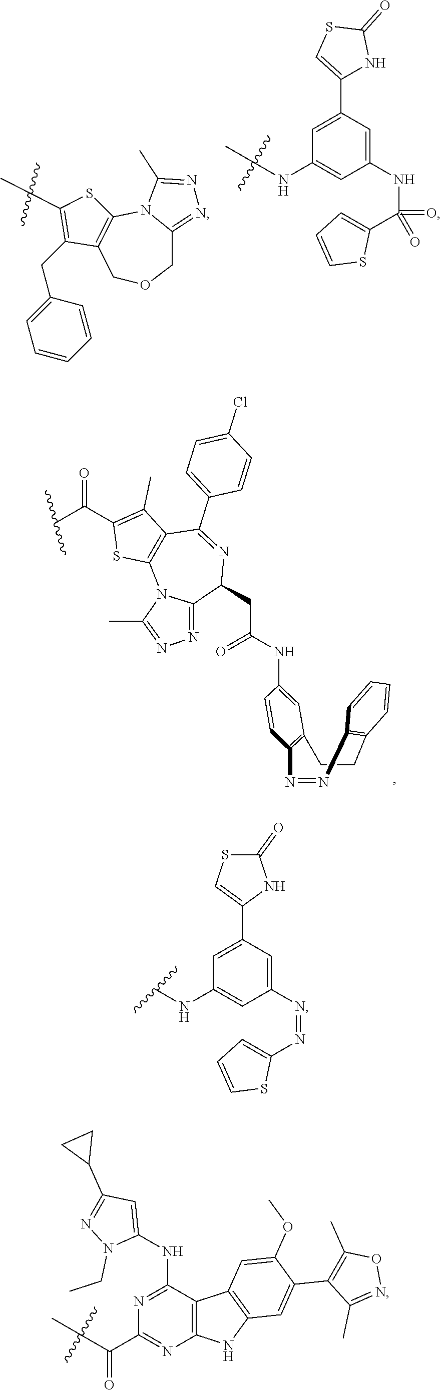 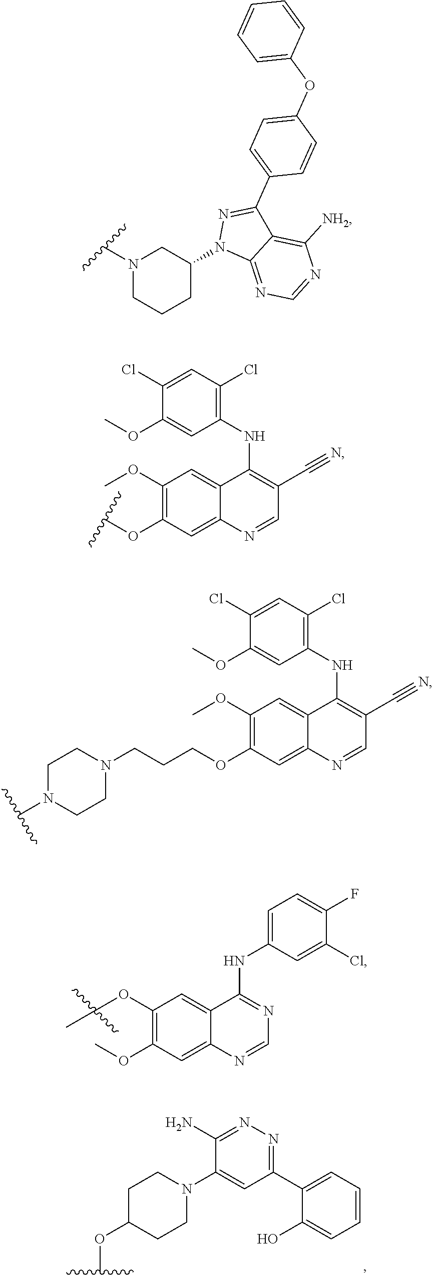 [SMILES: *N1CCC[C@@H](n2nc(-c3ccc(Oc4ccccc4)cc3)c3c(N)ncnc32)C1 ; *Oc1cc2ncc(C#N)c(Nc3cc(OC)c(Cl)cc3Cl)c2cc1OC ; *N1CCN(CCCOc2cc3ncc(C#N)c(Nc4cc(OC)c(Cl)cc4Cl)c3cc2OC)CC1 ; *Oc1cc2c(Nc3ccc(F)c(Cl)c3)ncnc2cc1OC ; *OC1CCN(c2cc(-c3ccccc3O)nnc2N)CC1] 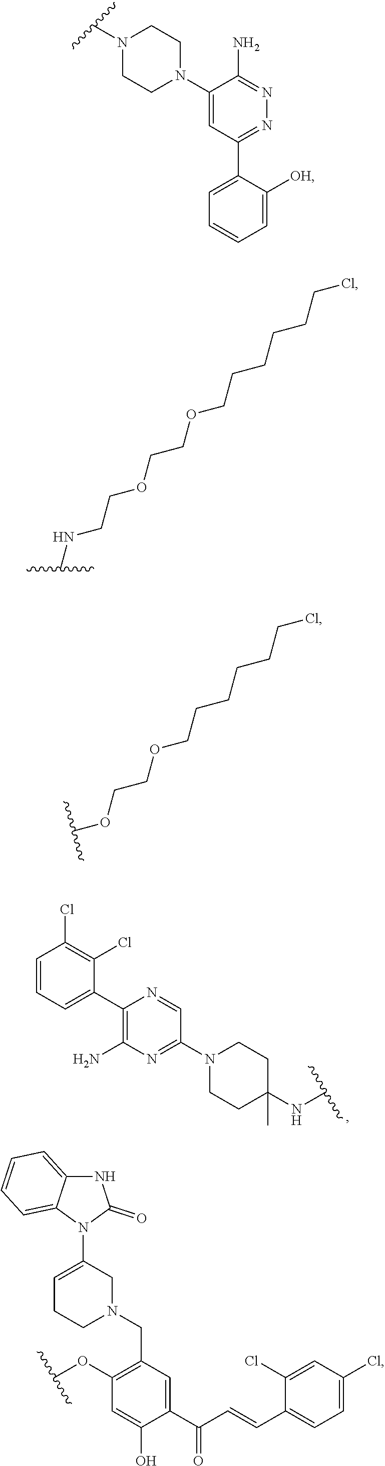 [SMILES: *N1CCN(c2cc(-c3ccccc3O)nnc2N)CC1 ; *NCCOCCOCCCCCCCl ; *OCCOCCCCCCCl ; *NC1(C)CCN(c2cnc(-c3cccc(Cl)c3Cl)c(N)n2)CC1 ; *Oc1cc(O)c(C(=O)/C=C/c2ccc(Cl)cc2Cl)cc1CN1CCC=C(n2c(=O)[nH]c3ccccc32)C1] 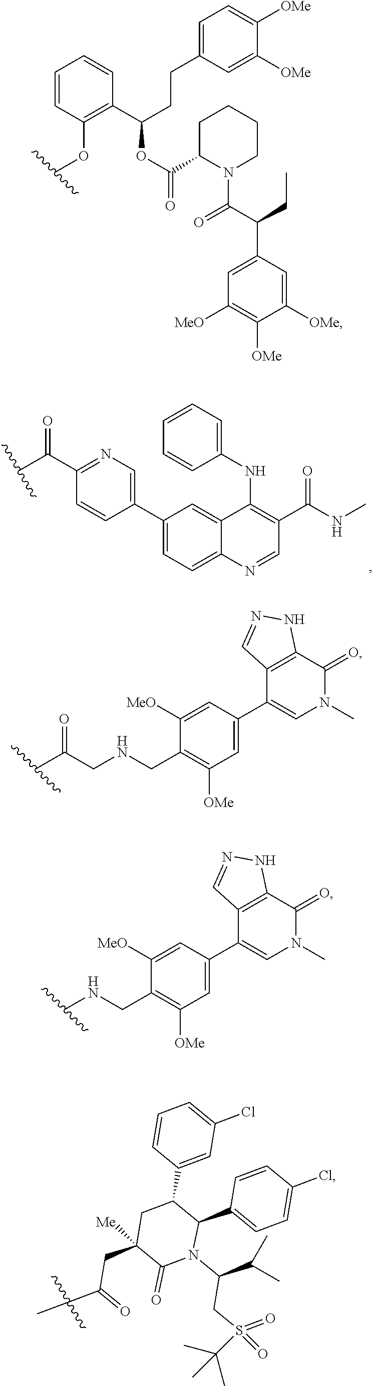 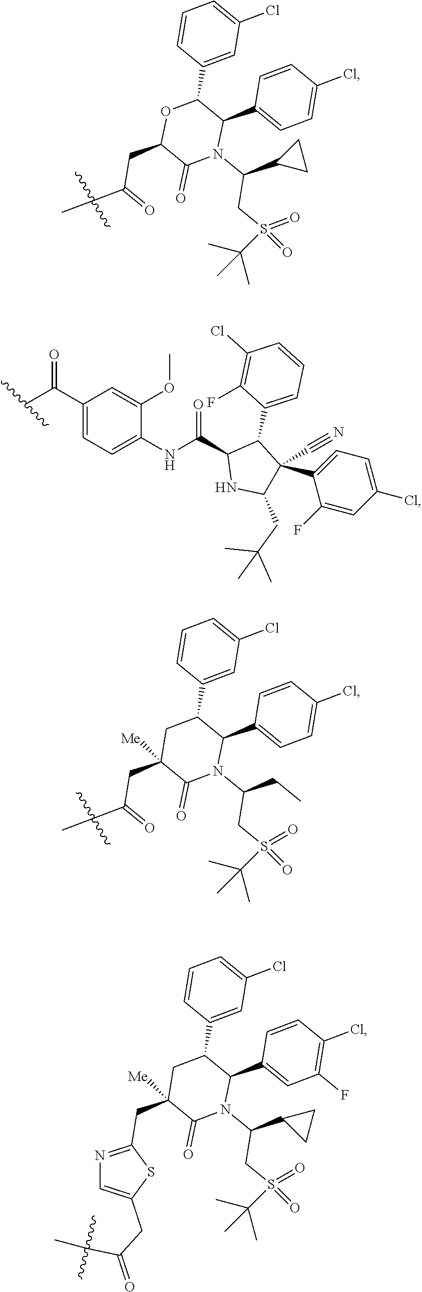 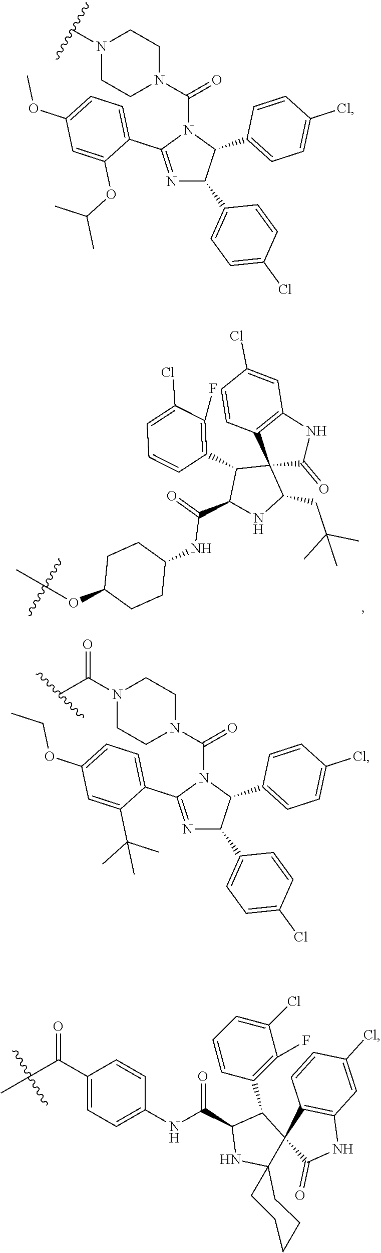 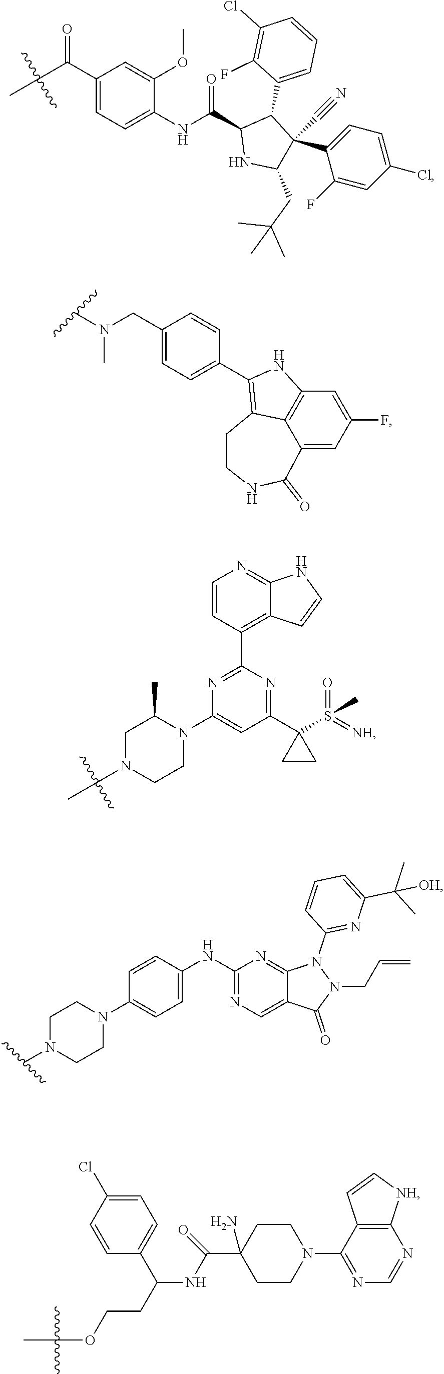 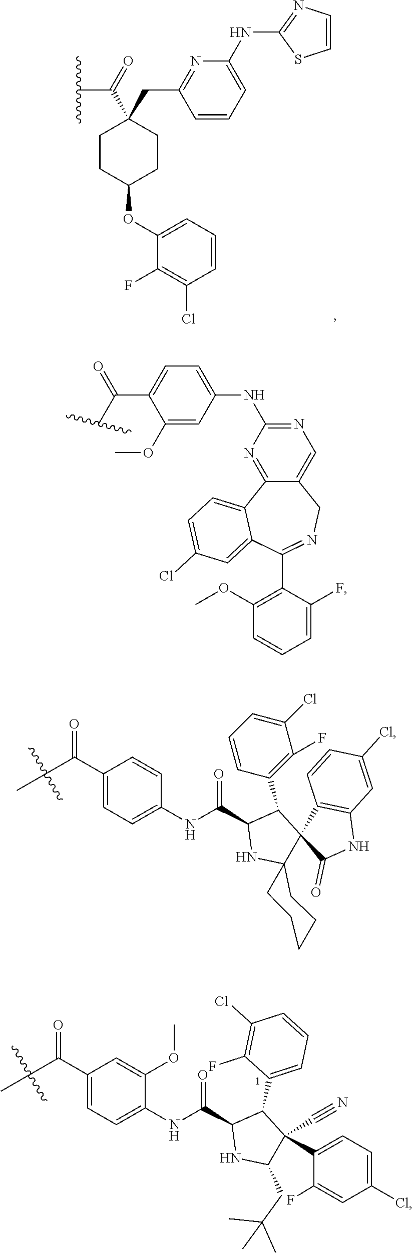 [SMILES: *C(=O)[C@]1(Cc2cccc(Nc3nccs3)n2)CC[C@@H](Oc2cccc(Cl)c2F)CC1 ; *C(=O)c1ccc(Nc2ncc3c(n2)-c2ccc(Cl)cc2C(c2c(F)cccc2OC)=NC3)cc1OC ; *C(=O)c1ccc(NC(=O)[C@@H]2NC3(CCCCC3)[C@]3(C(=O)Nc4cc(Cl)ccc43)[C@H]2c2cccc(Cl)c2F)cc1 ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]
and GQEDATADDQYQQY (SEQ ID NO:4).

7. A compound of claim 1, wherein the compound has the following structure: 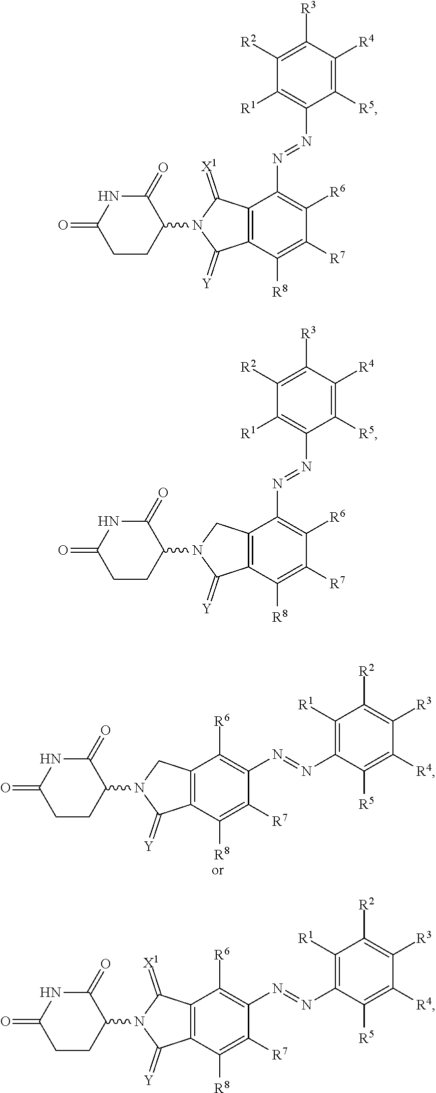
wherein Y and X¹ are independently chosen from O and S; R¹-R⁷ are each independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, and B(O-aryl)₂ and wherein R¹-R⁵ is also chosen from L-B*, wherein L is optional and B* is a ligand for a target protein.

8. A compound of claim 1, wherein the compound has the following structure: 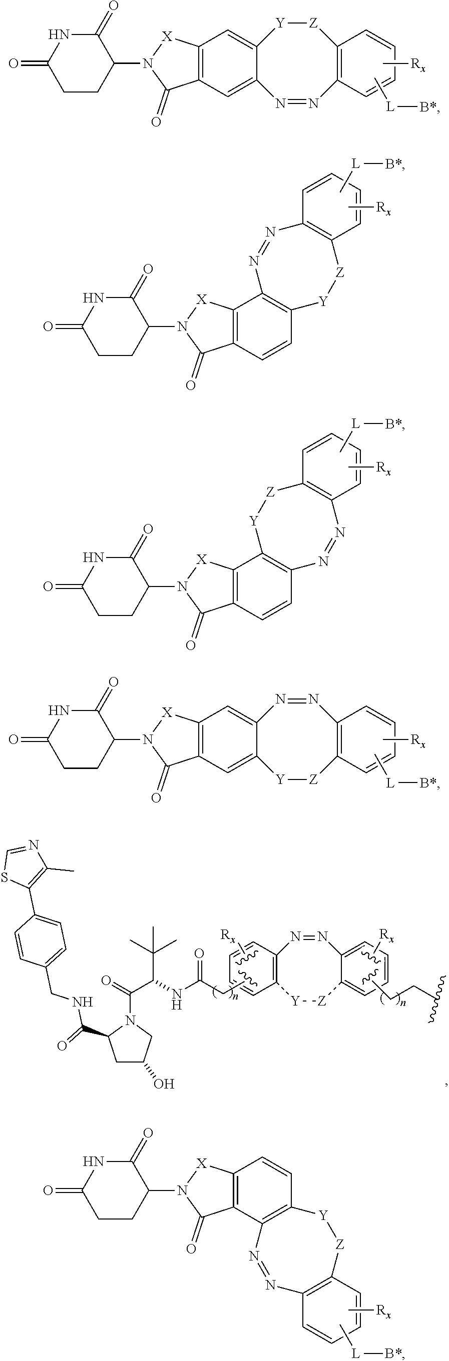 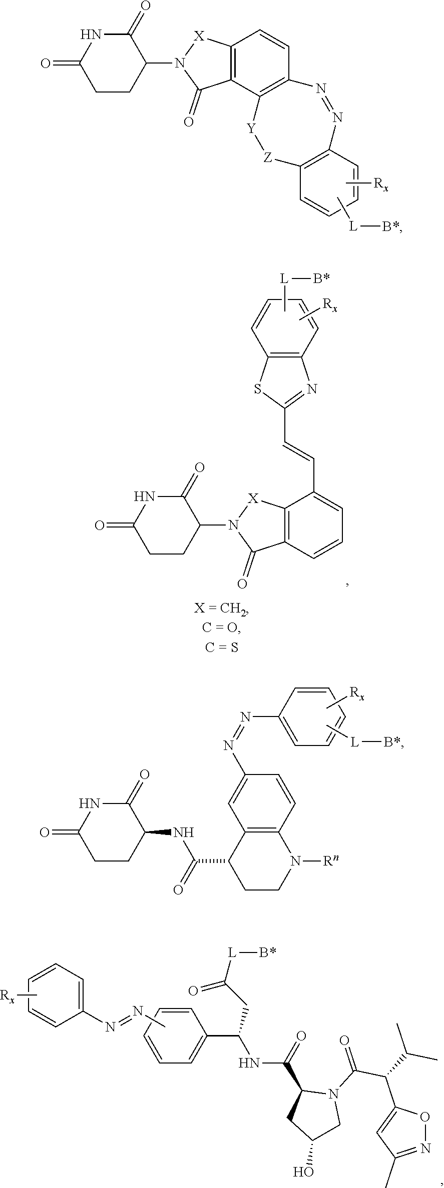 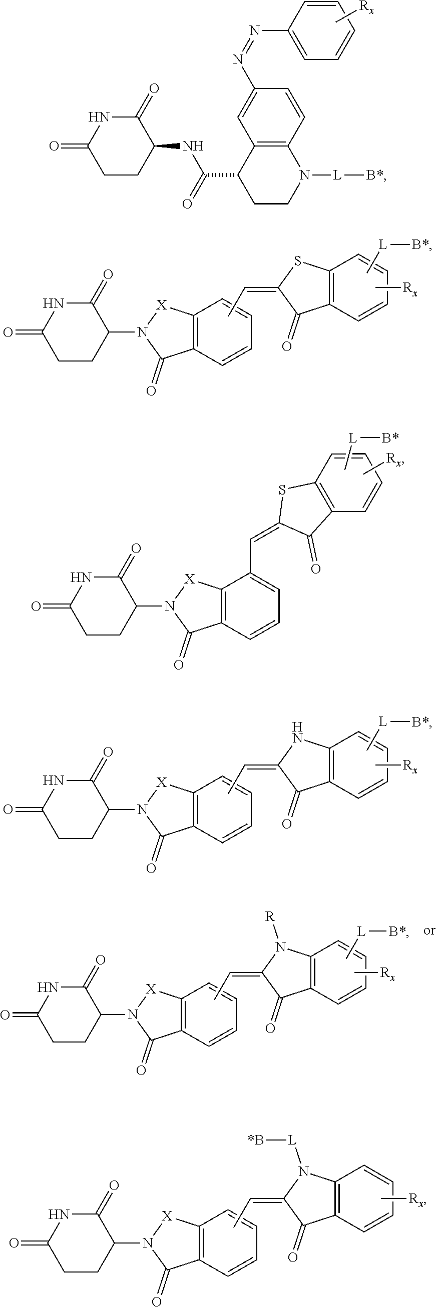
wherein X is methylene, C═O, or C═S; Y and Z are independently methylene, O, or S; each R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, SO₂H, SO₂-alkyl, SO₂-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, and L-B*, wherein L is optional and B* is a ligand for a target protein, and x is 0, 1, 2, 3, 4, or 5.

9. A compound of claim 1, wherein the compound has the following structure: 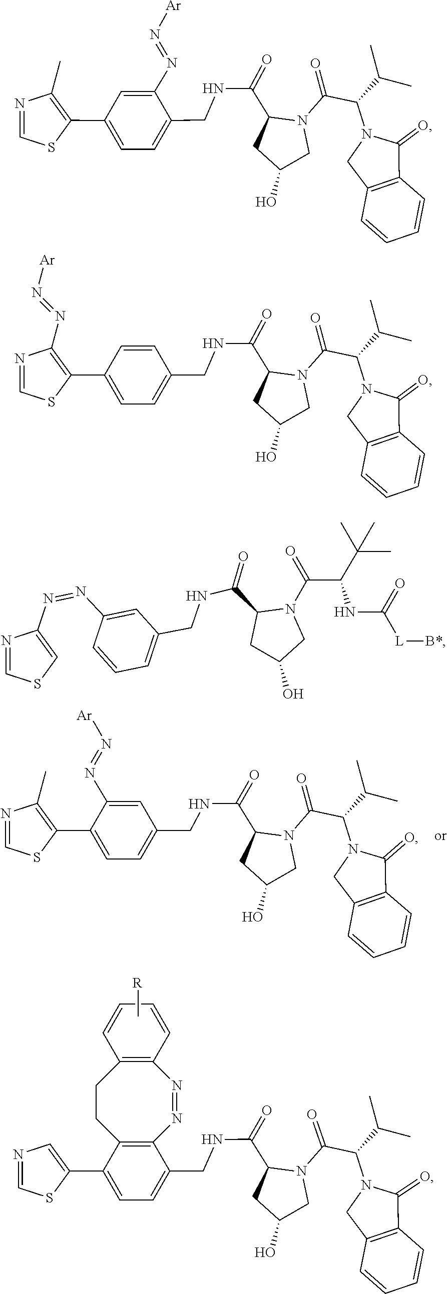
wherein R is independently chosen from H, halogen, alkyl, S-alkyl, NH-alkyl, cycloalkyl, aryl, heteroaryl, heterocyclyl, OH, O-alkyl, O-aryl, NH₂, NH-aryl, N(alkyl)₂, N(alkyl)(aryl), N(alkyl)(cycloalkyl), N(aryl)(cycloalkyl), N(cycloalkyl)₂, N(aryl)₂, SH, 502H, 502-alkyl, 502-aryl, SO₃H, P(aryl)₂, P(O)(aryl)₂, P(O)(O-alkyl)₂, CCH, CH═CH(alkyl), CH═C(alkyl)₂, Si(alkyl)₃, Si(aryl)₃, NH(CO)NH₂, NH(CO)NH-alkyl, NH(CO)NH-aryl, NH(CS)NH-alkyl, NH(CS)NH-aryl, SO₂NH₂, SO₂NH-alkyl, SO₂NH-aryl, CN, CO₂H, C(O)alkyl, C(O)aryl, CO₂alkyl, CO₂aryl, C(O)NH₂, C(O)NH-alkyl, C(O)NH-aryl, C(O)N(alkyl)₂, C(O)N(aryl)₂, CF₃, CF₂H, CH₂F, NO₂, SF₅, OCF₃, CC-alkyl, CC-aryl, CO₂H, B(OH)₂, B(O-alkyl)₂, B(O-aryl)₂, and L-B*, wherein L is optional and is a linker and B* is a ligand for a target protein; and Ar is an aryl group chosen from 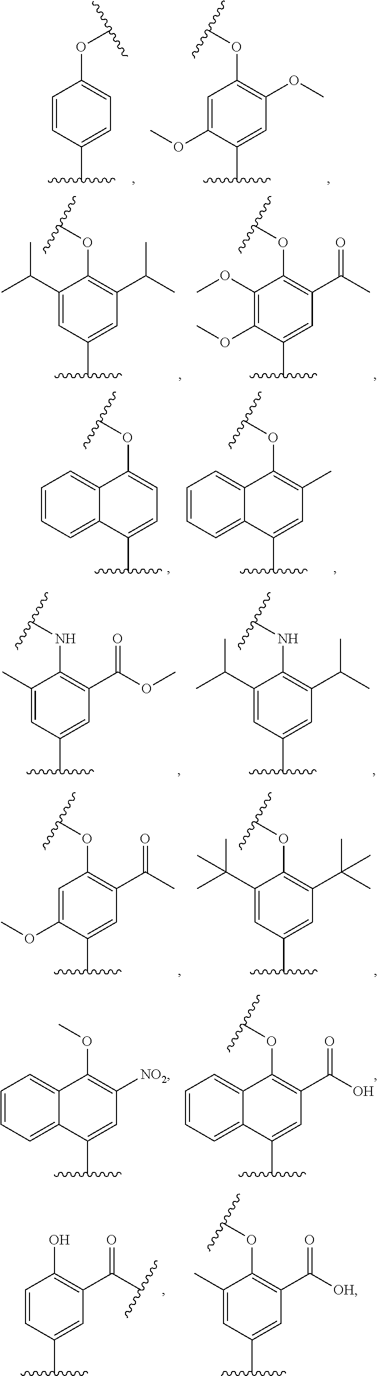 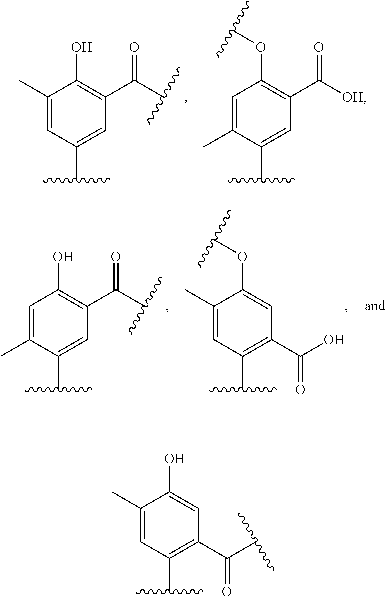
that is further attached to a linker that is attached to a ligand for a target protein.

10. A compound of claim 1, wherein the compound has the following structure:

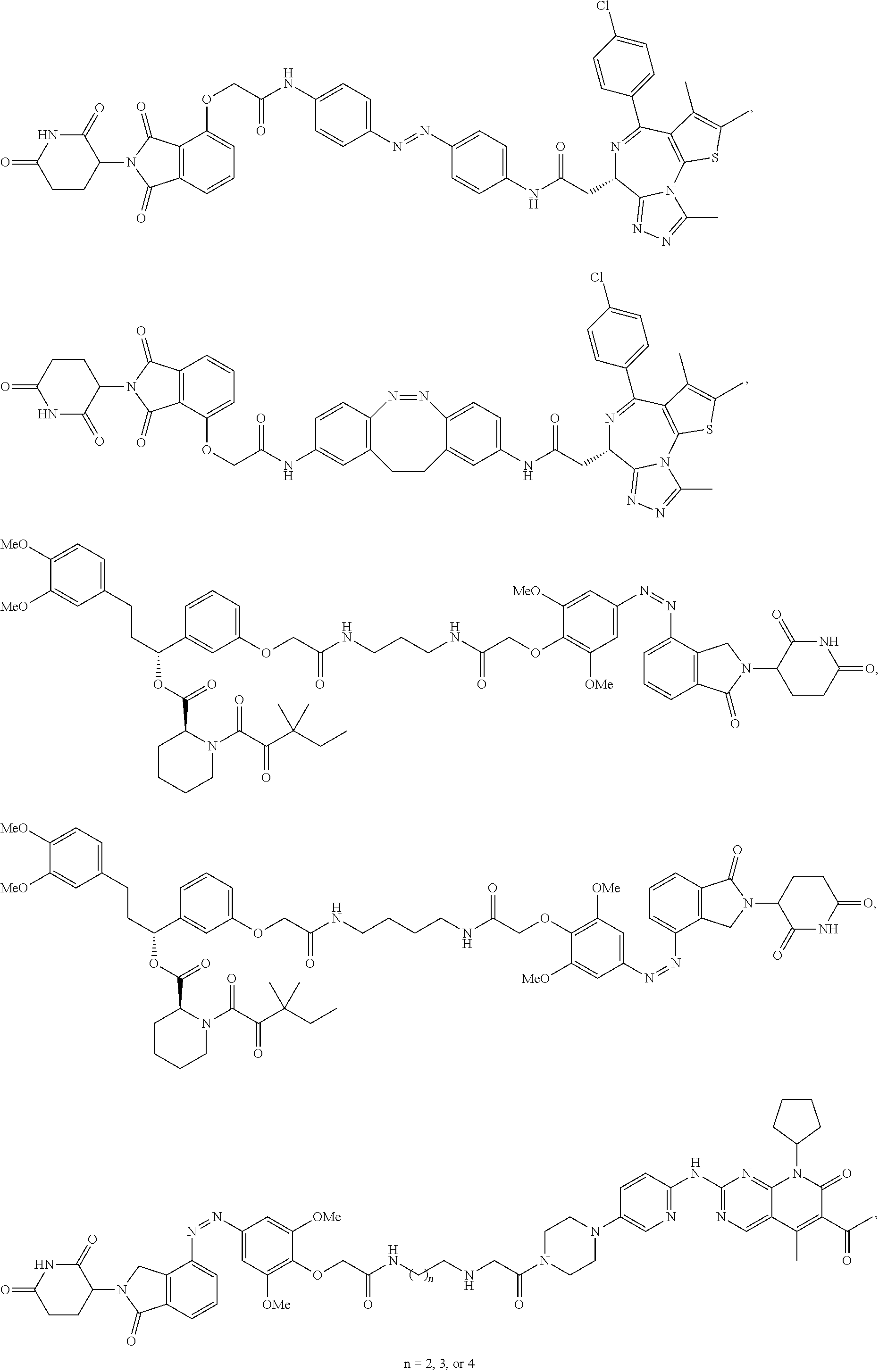

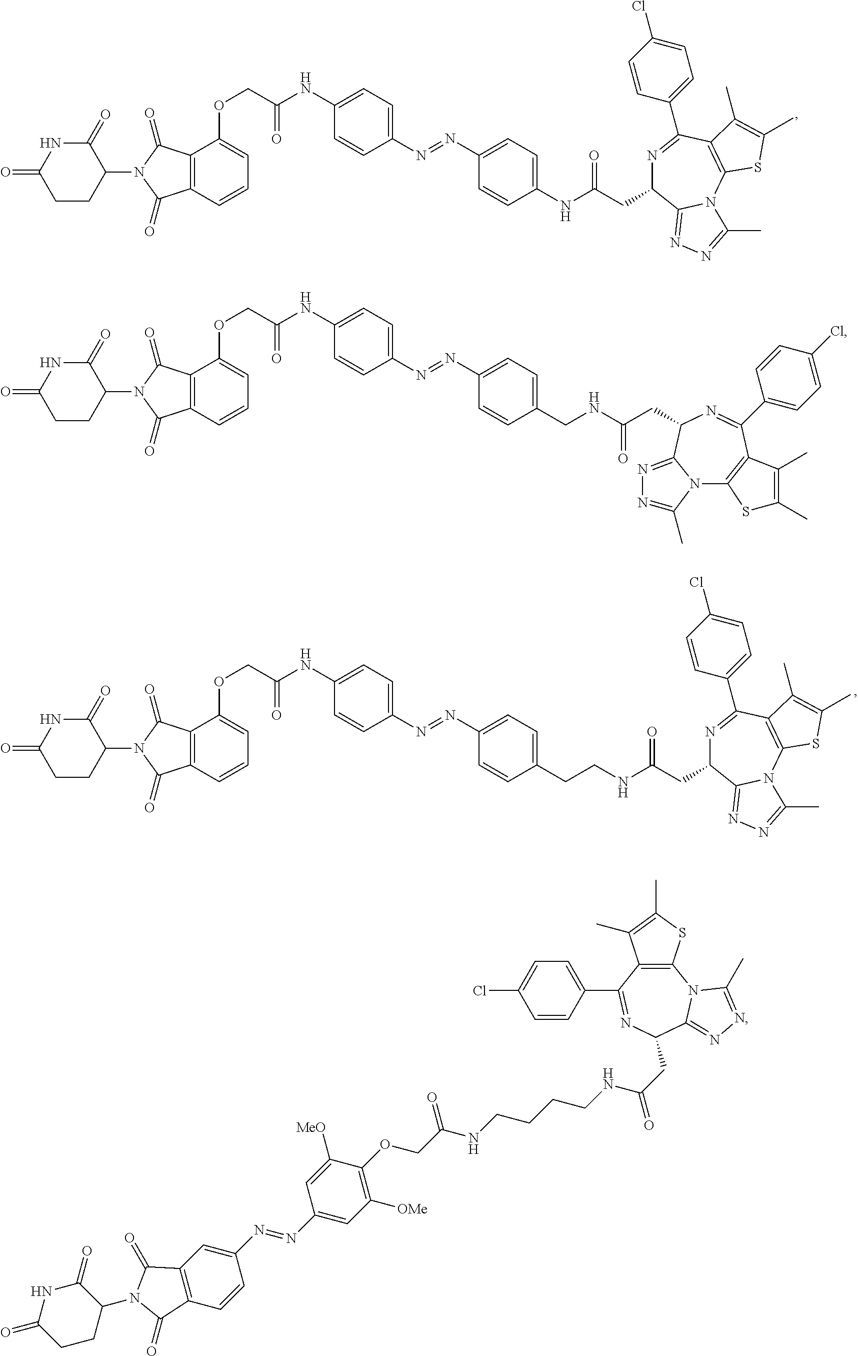

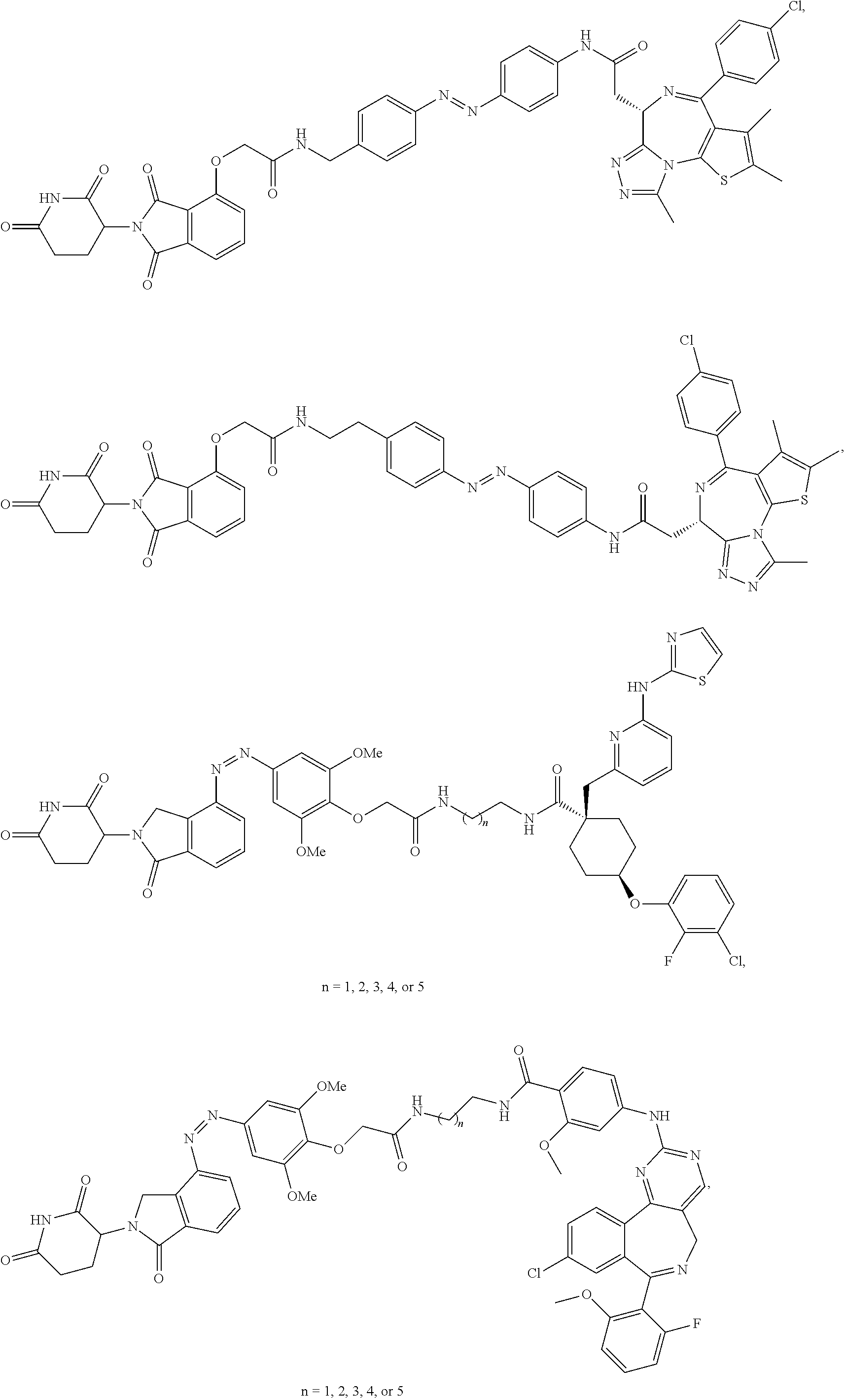

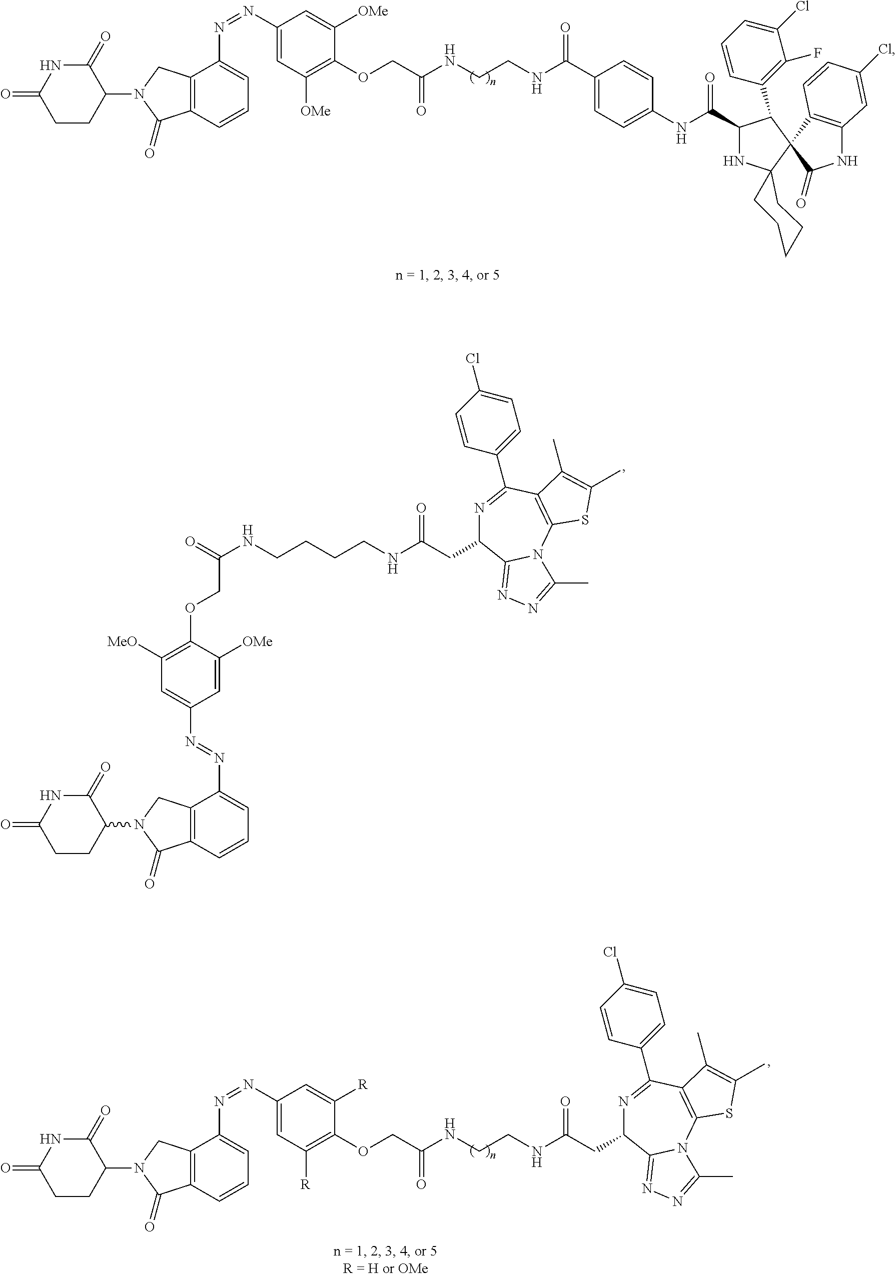

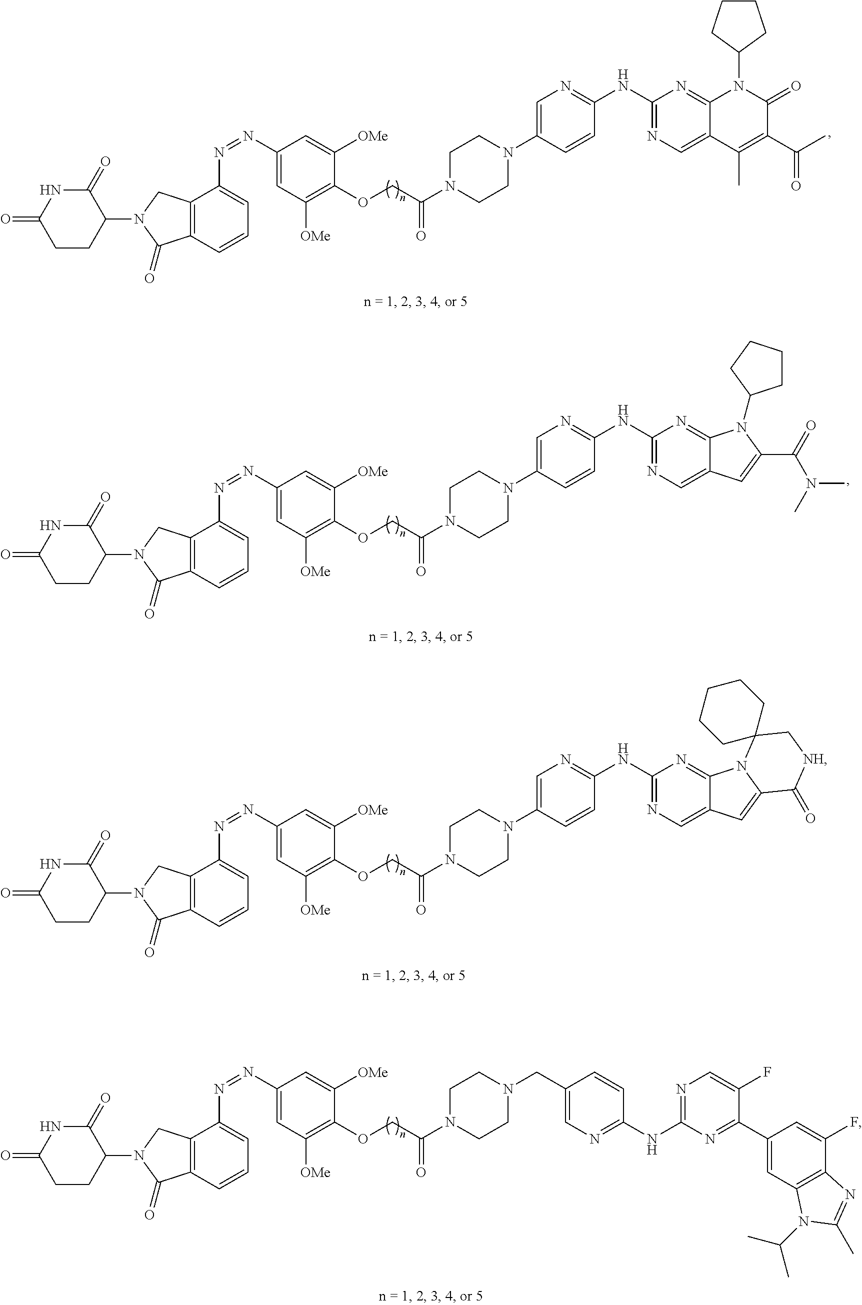

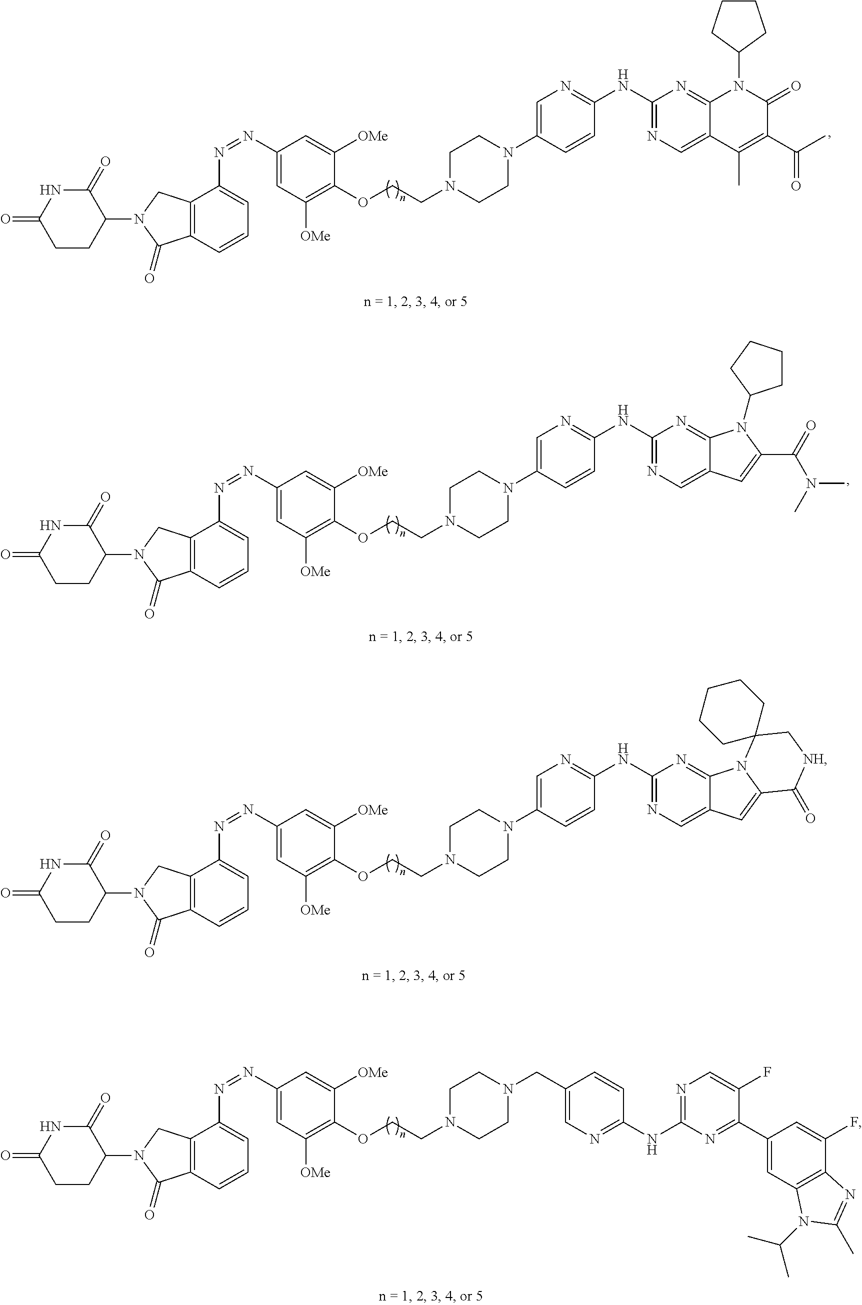

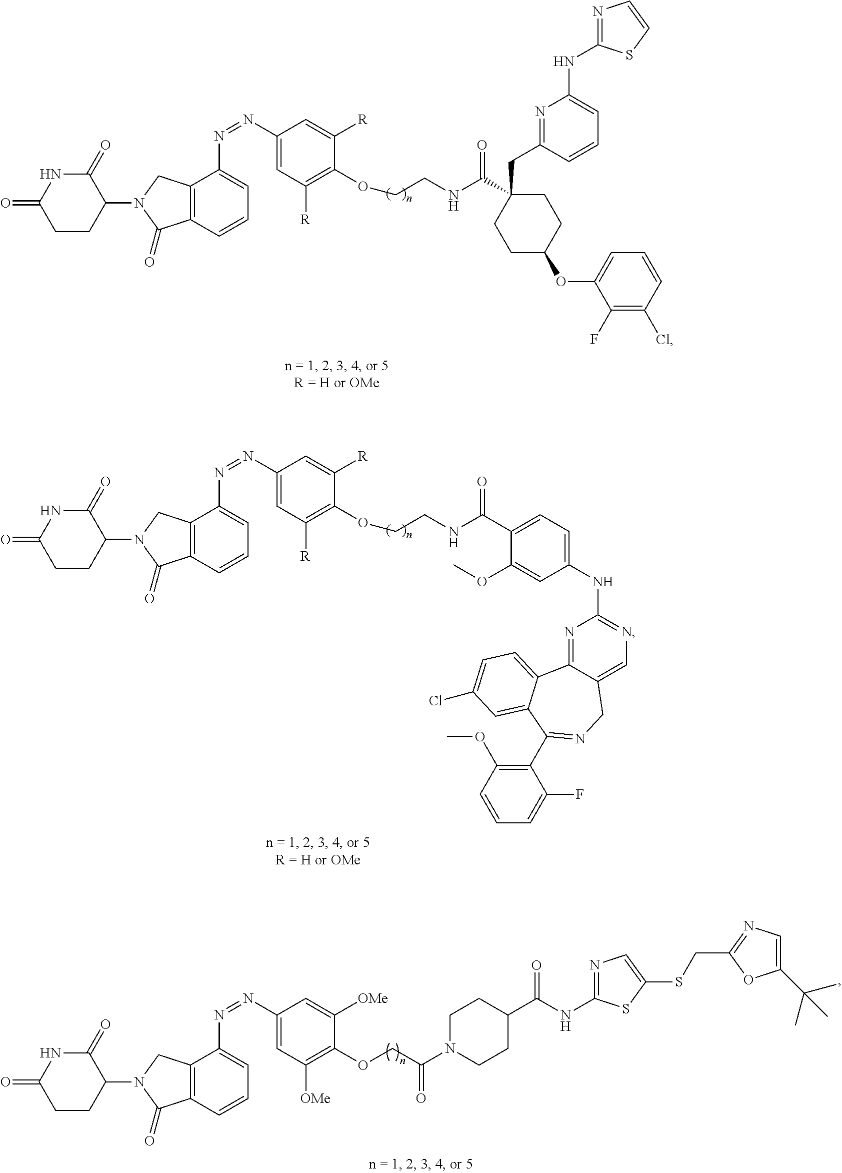

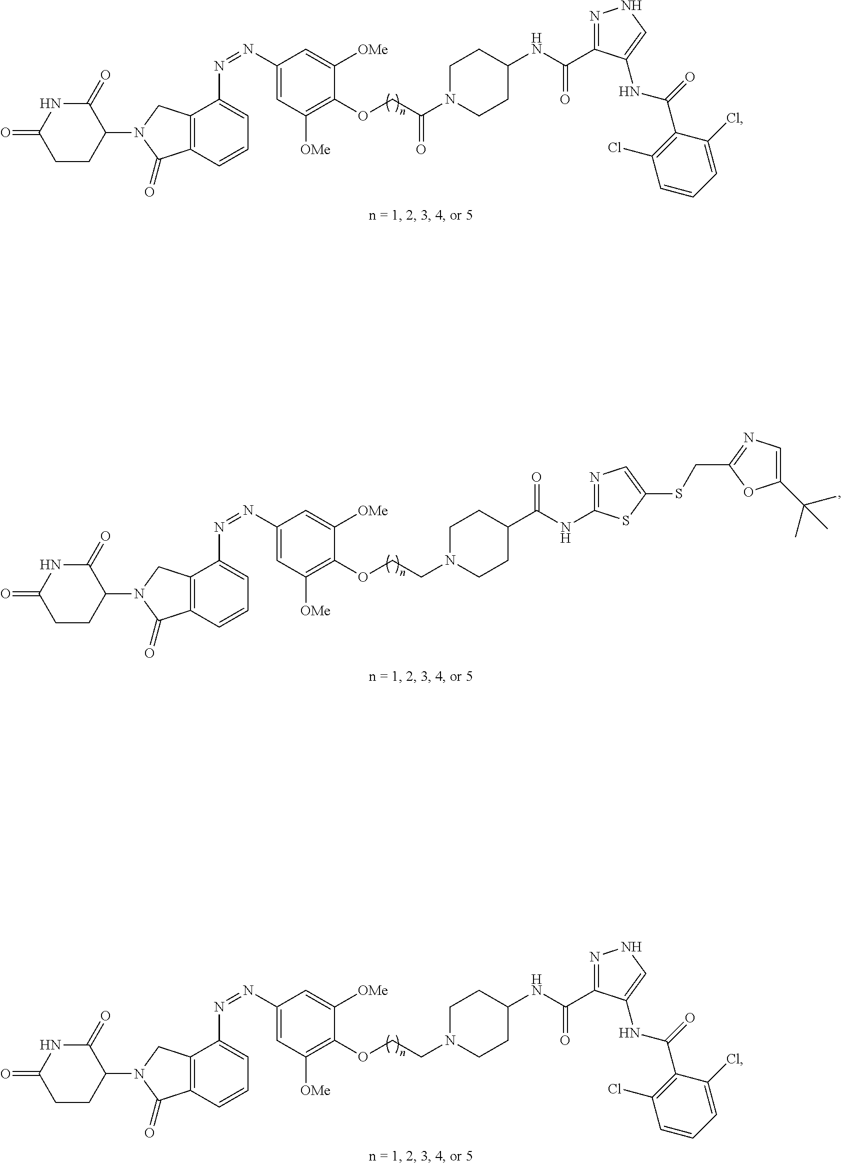

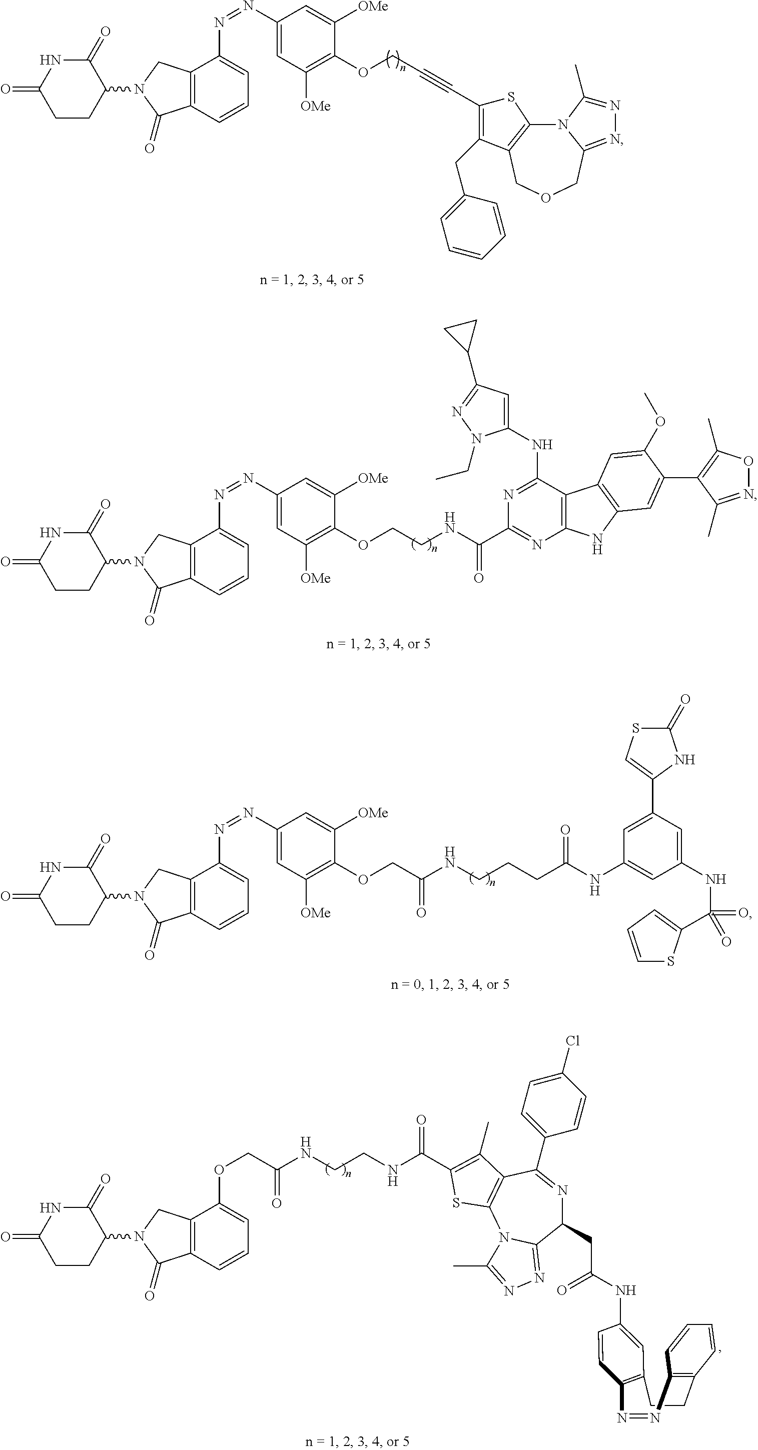

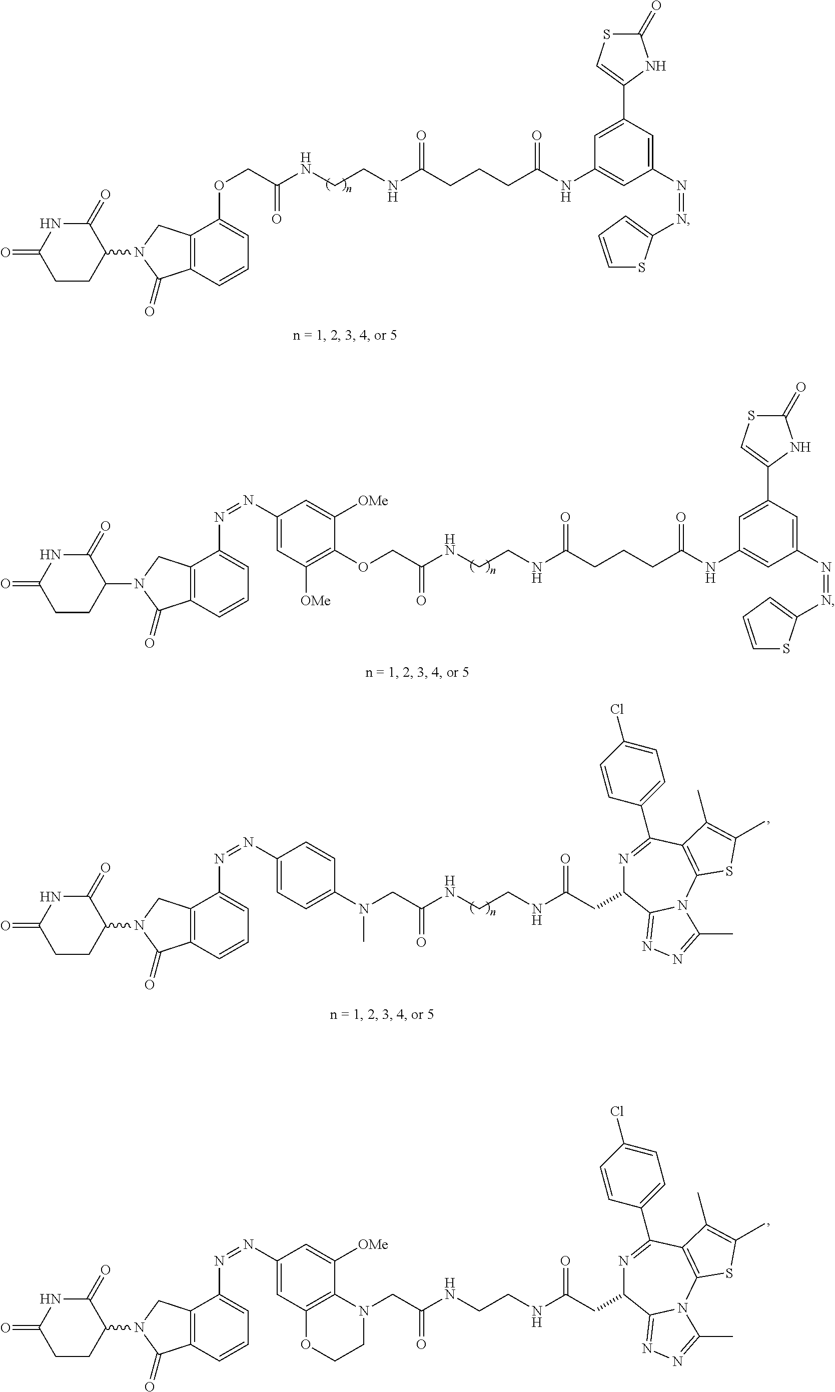

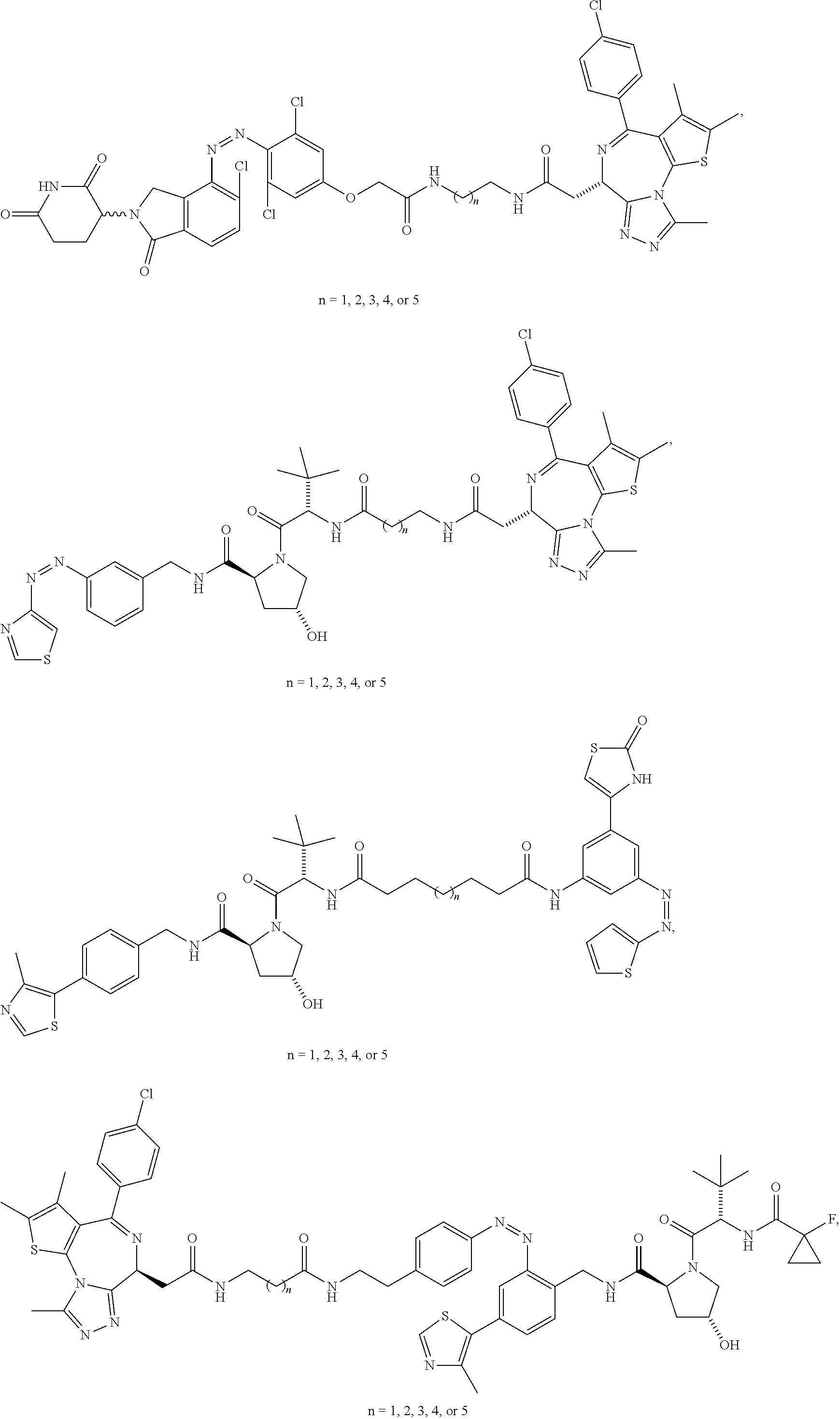

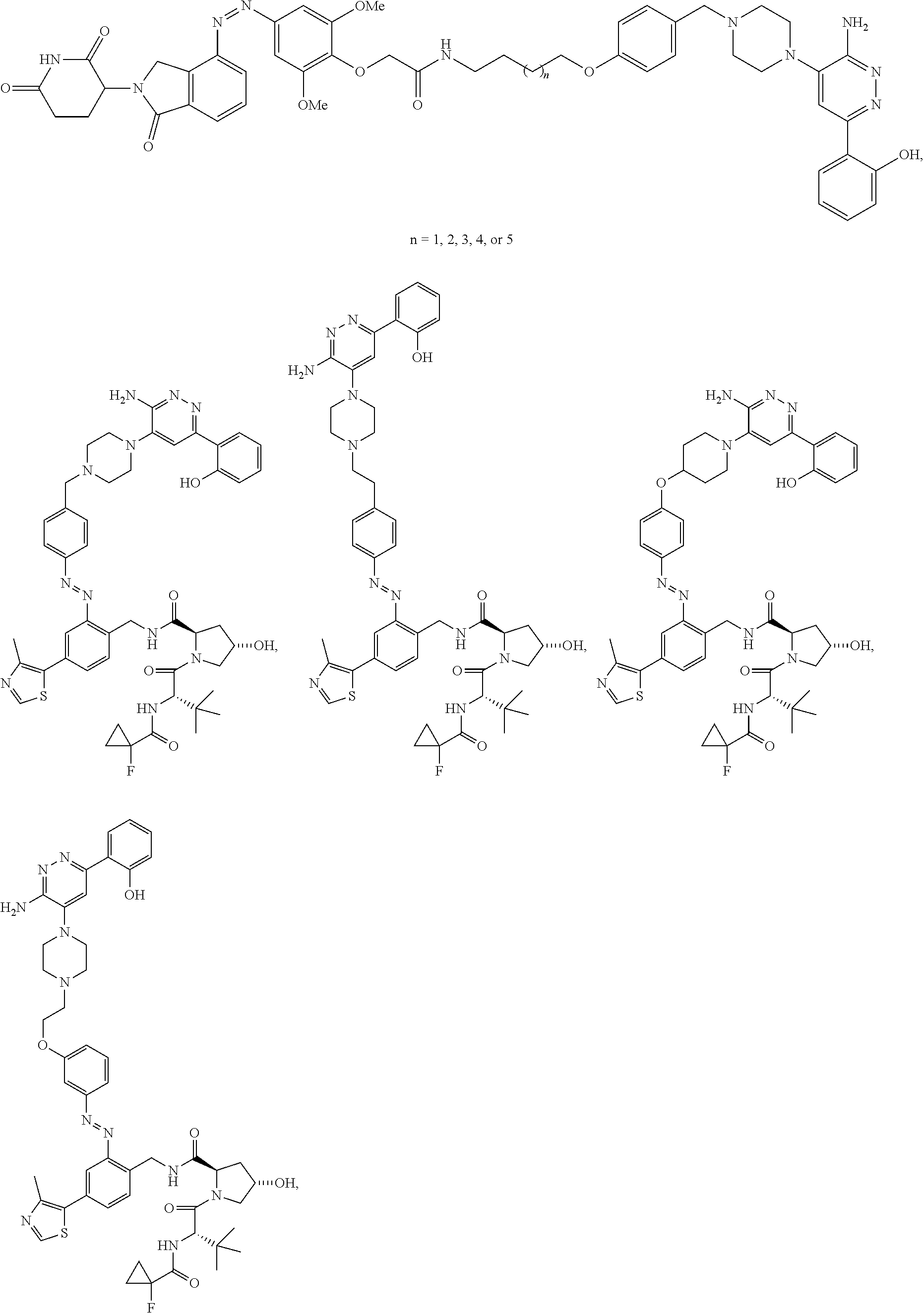

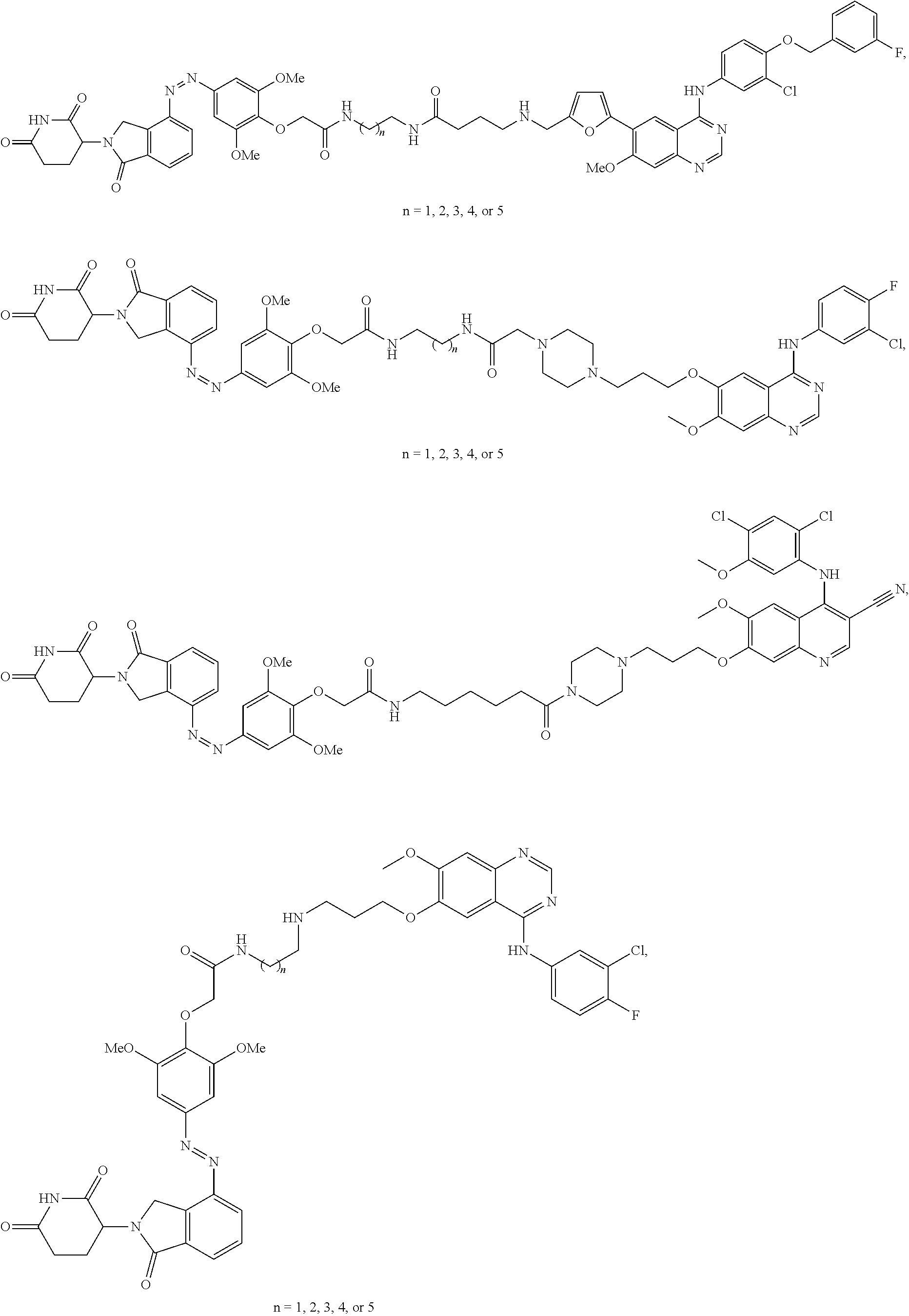

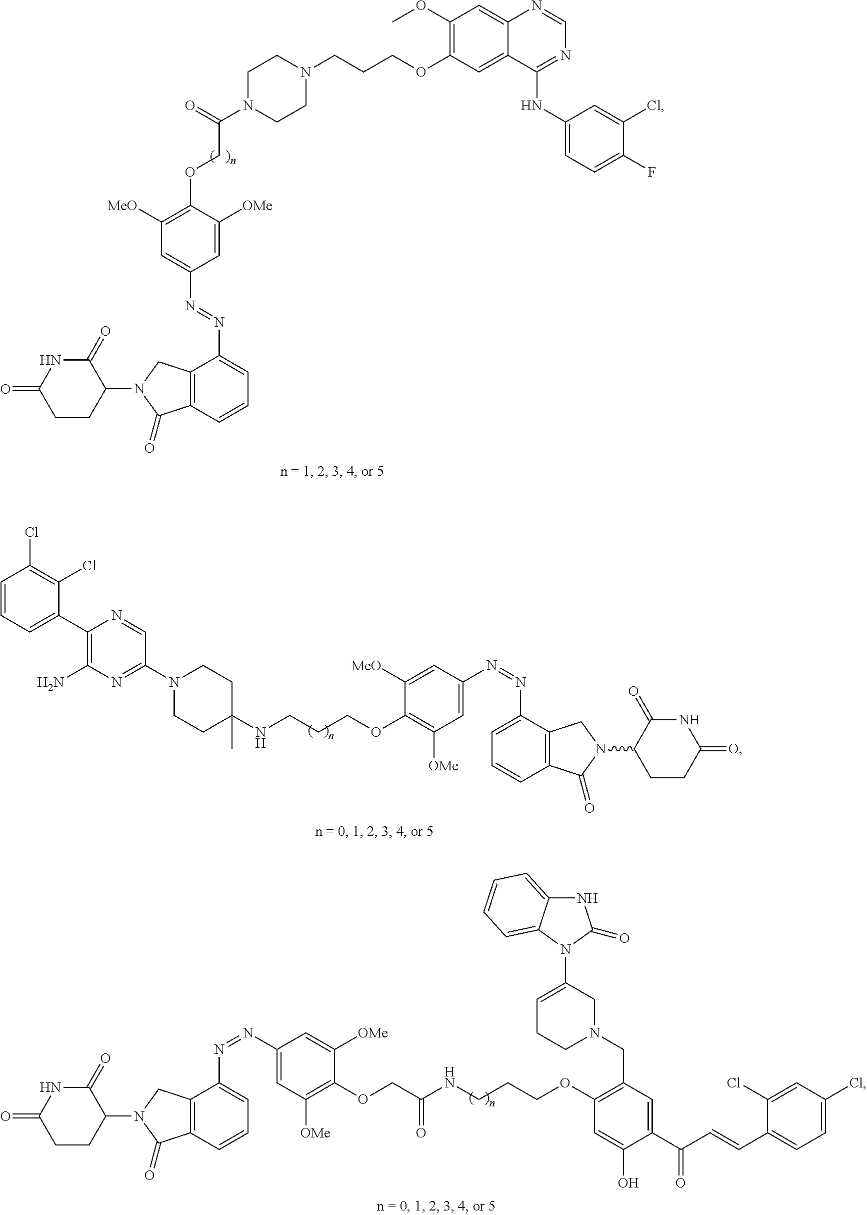

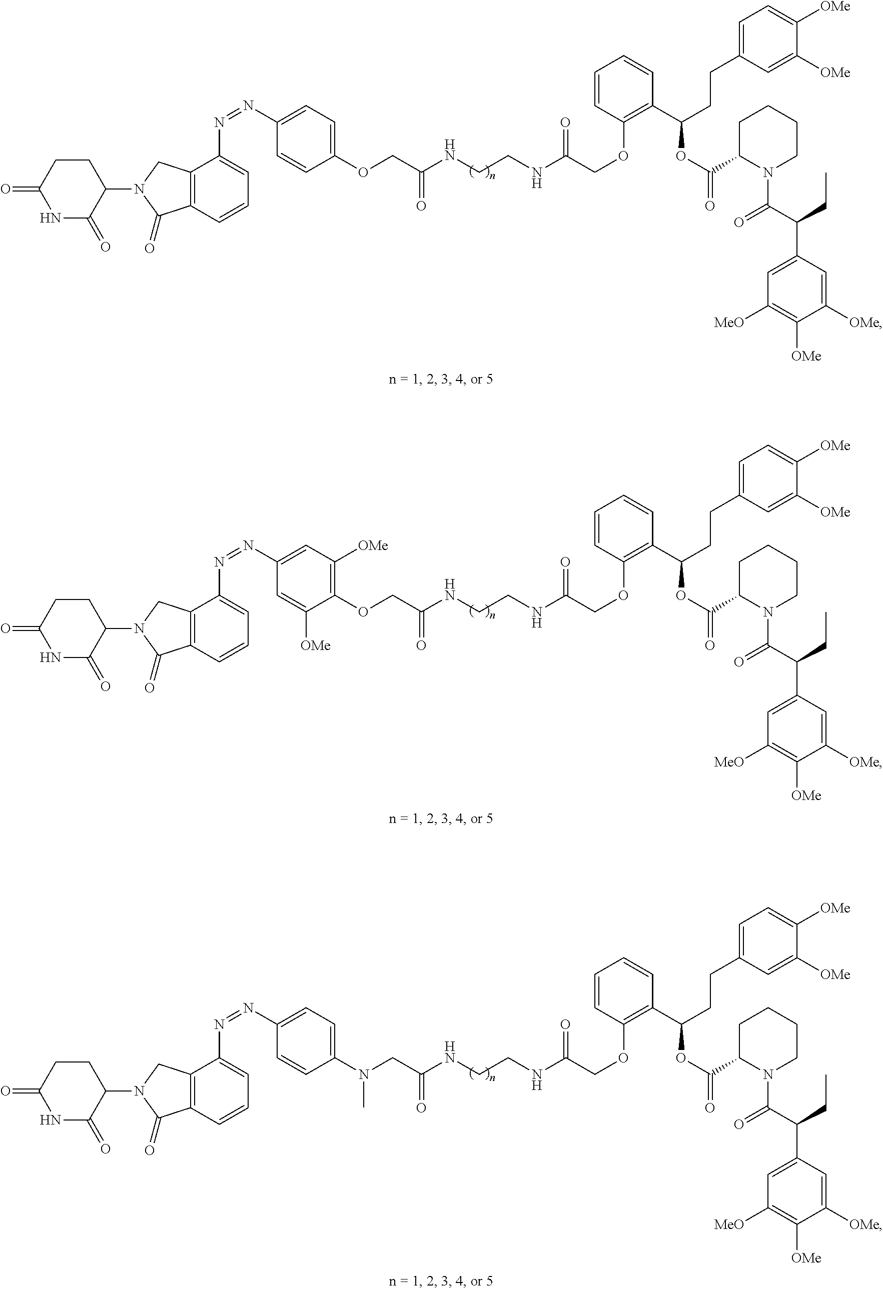

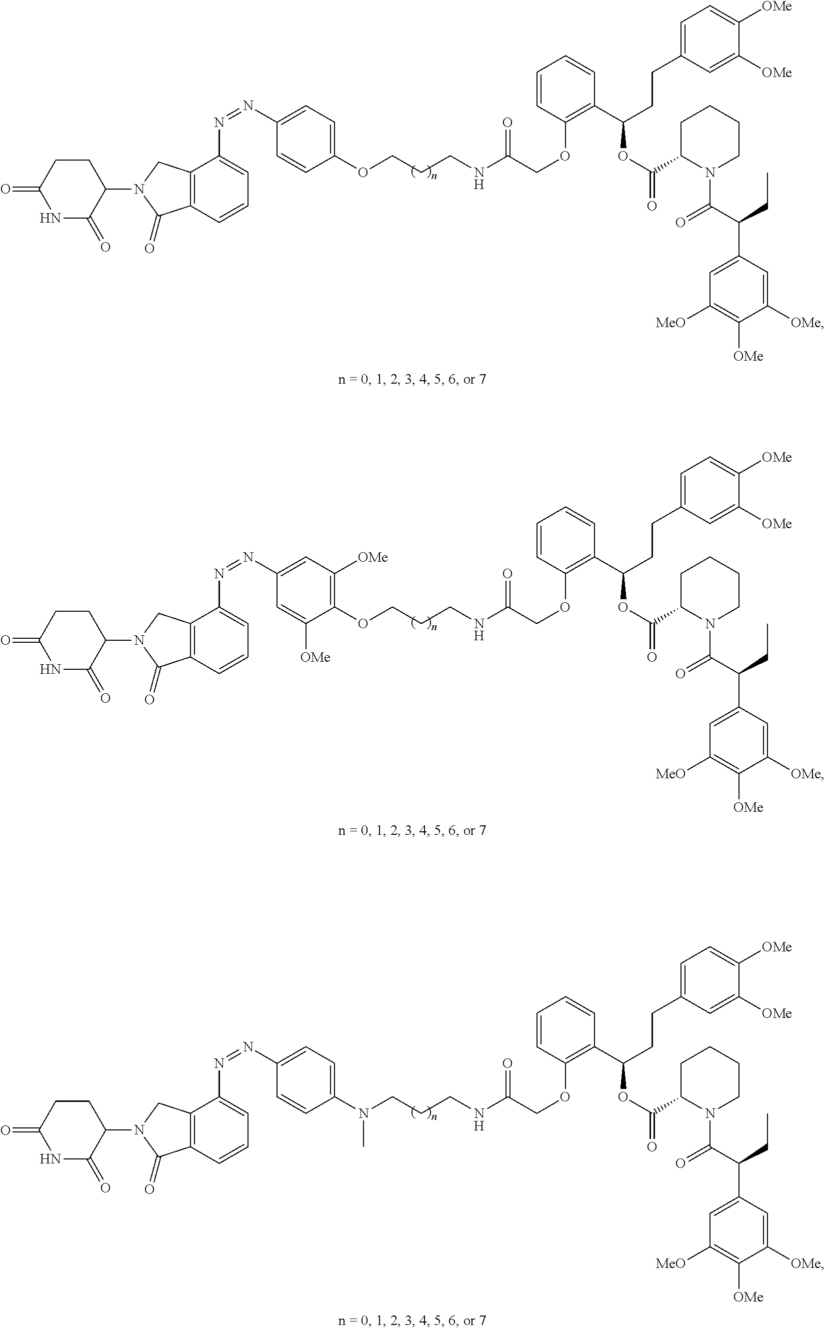

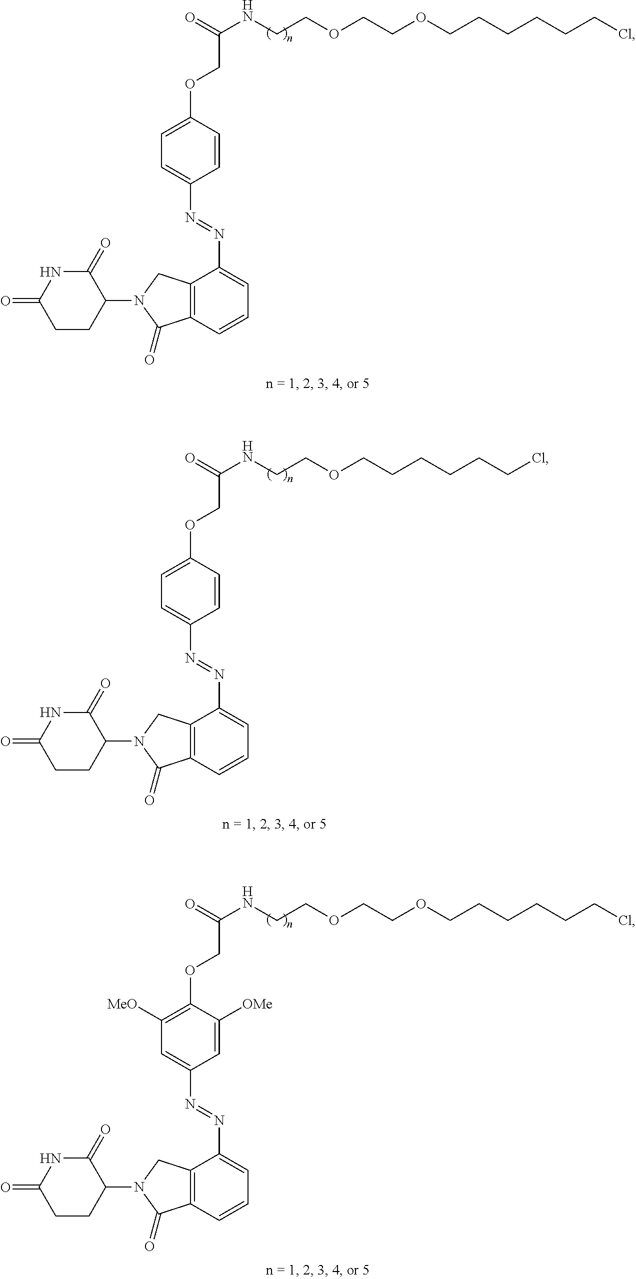

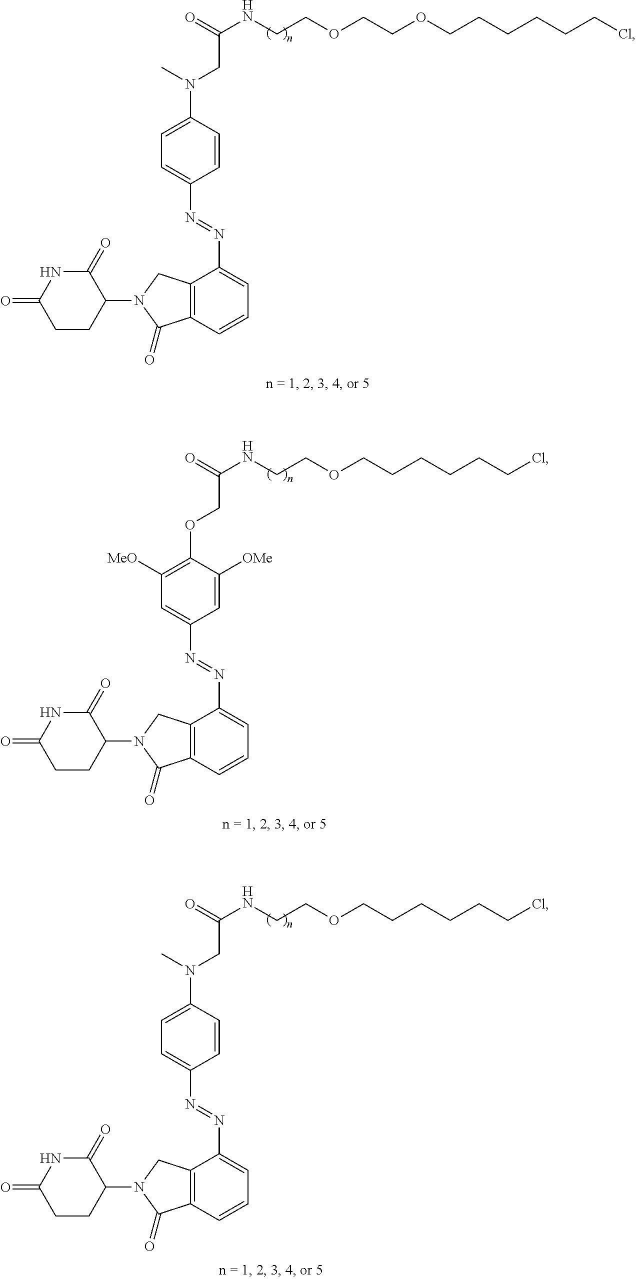

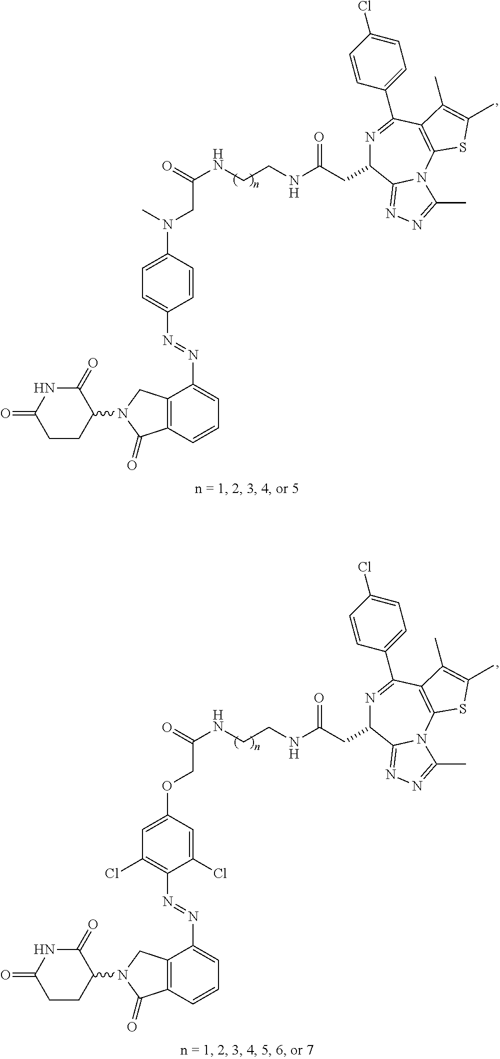

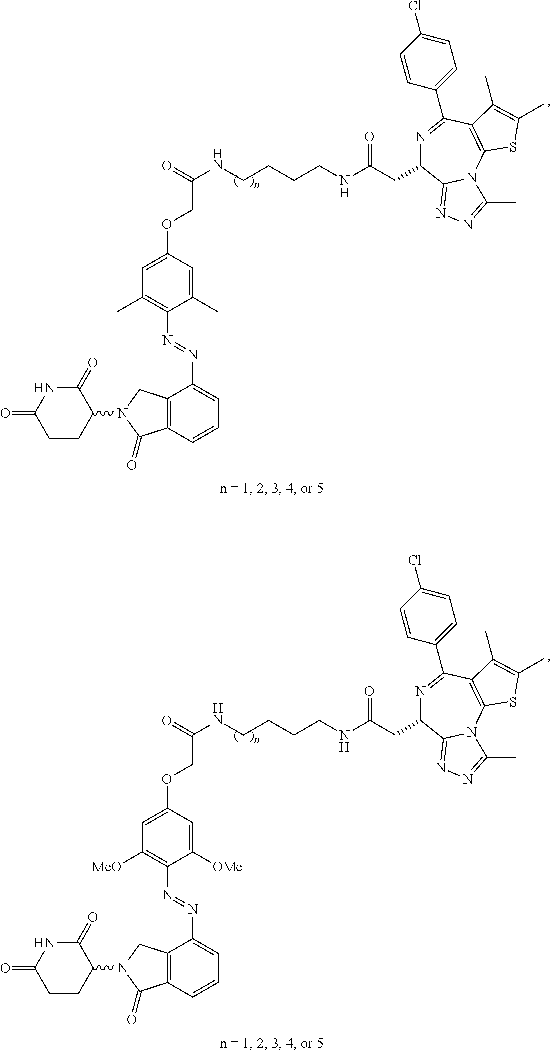

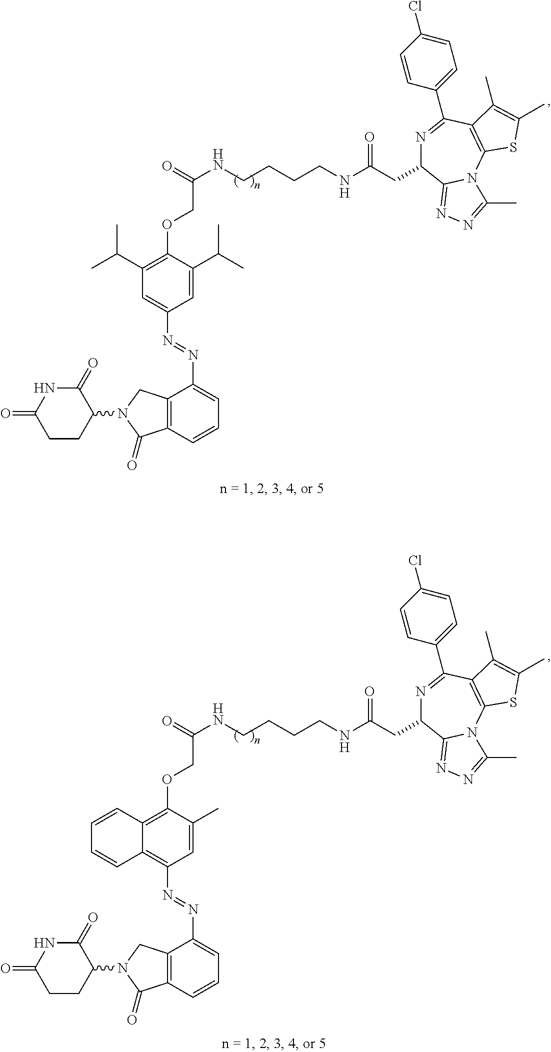

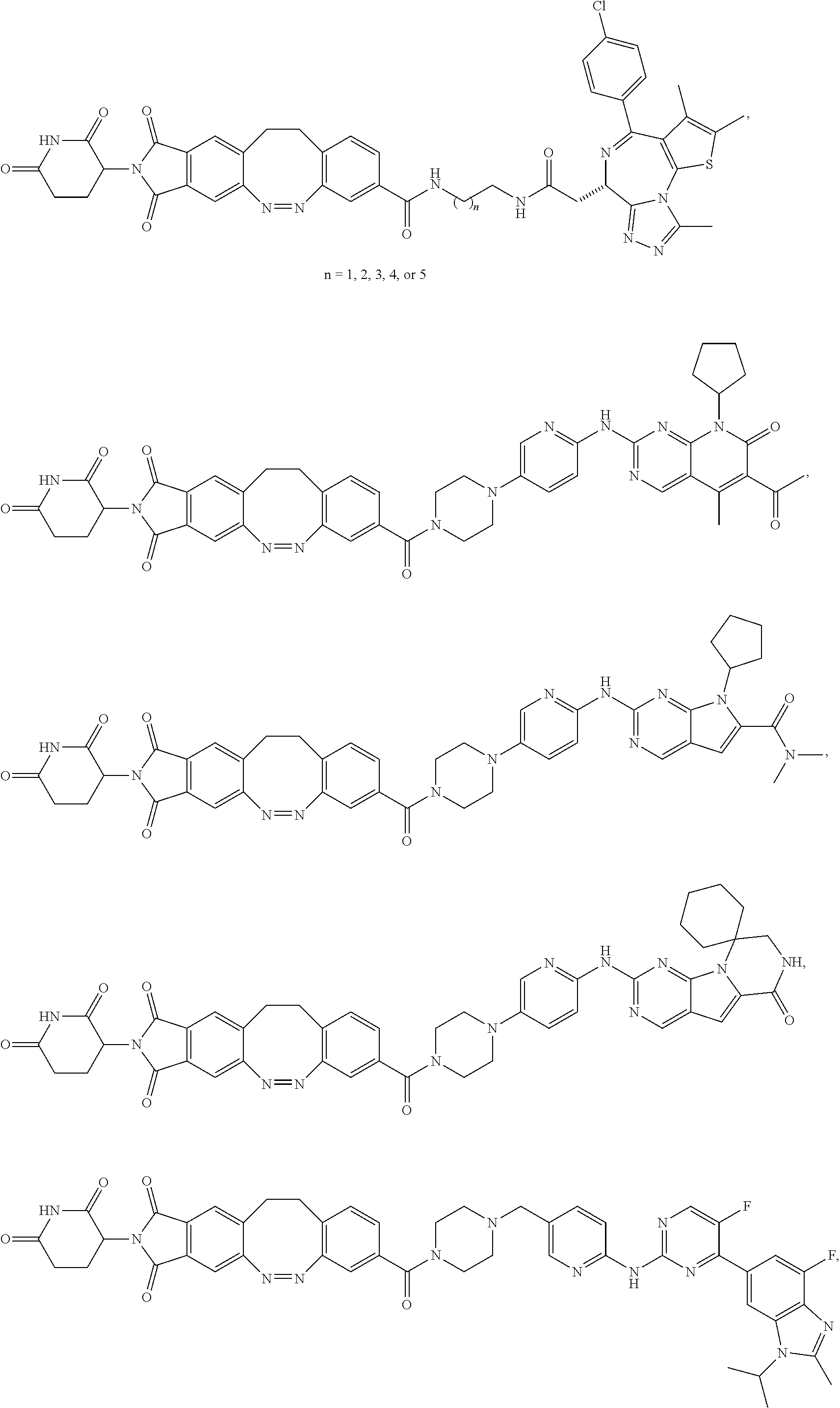

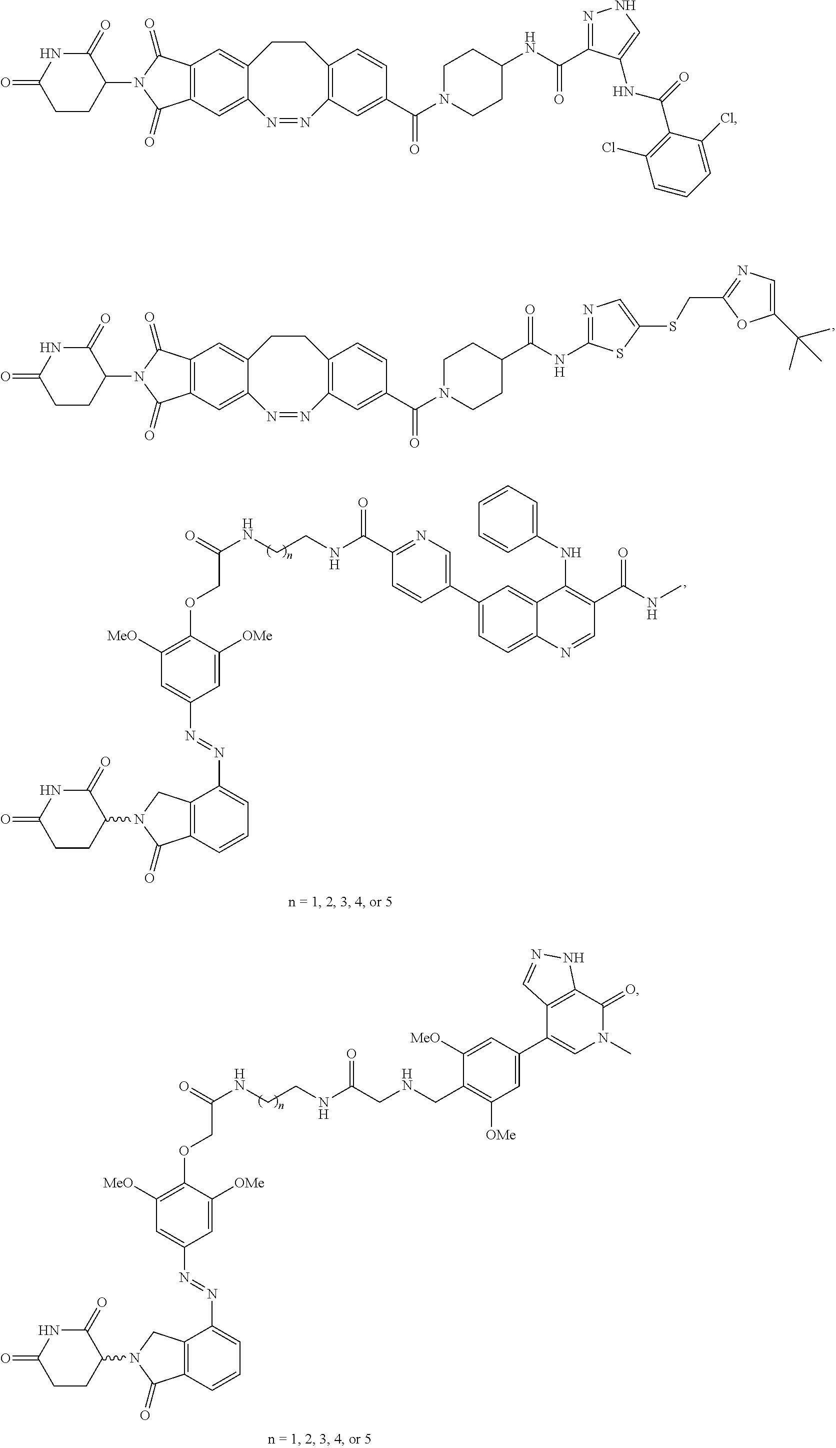

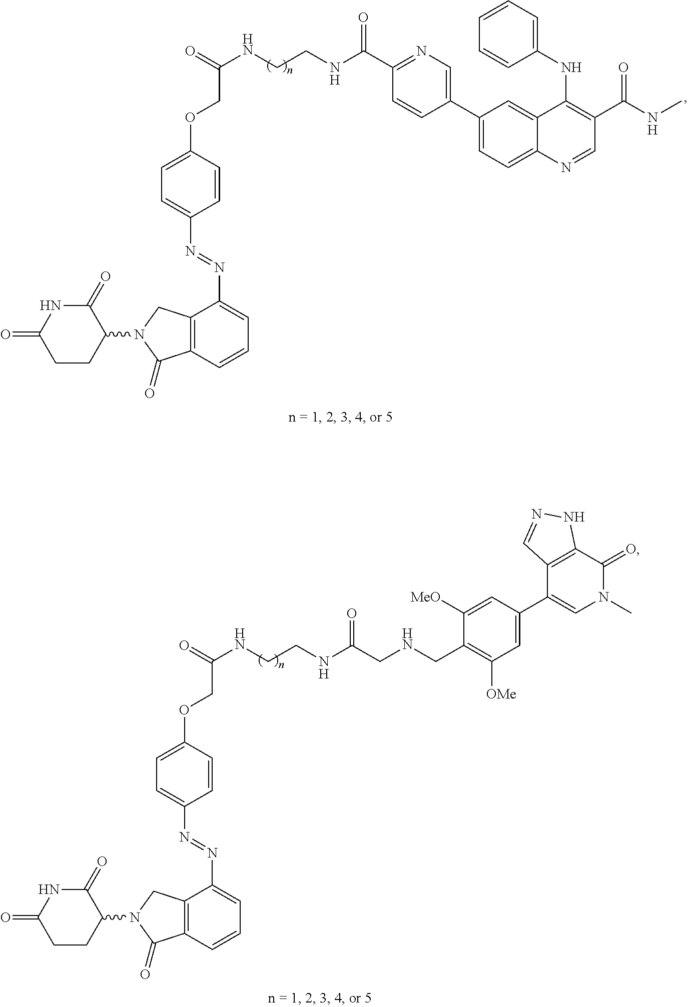

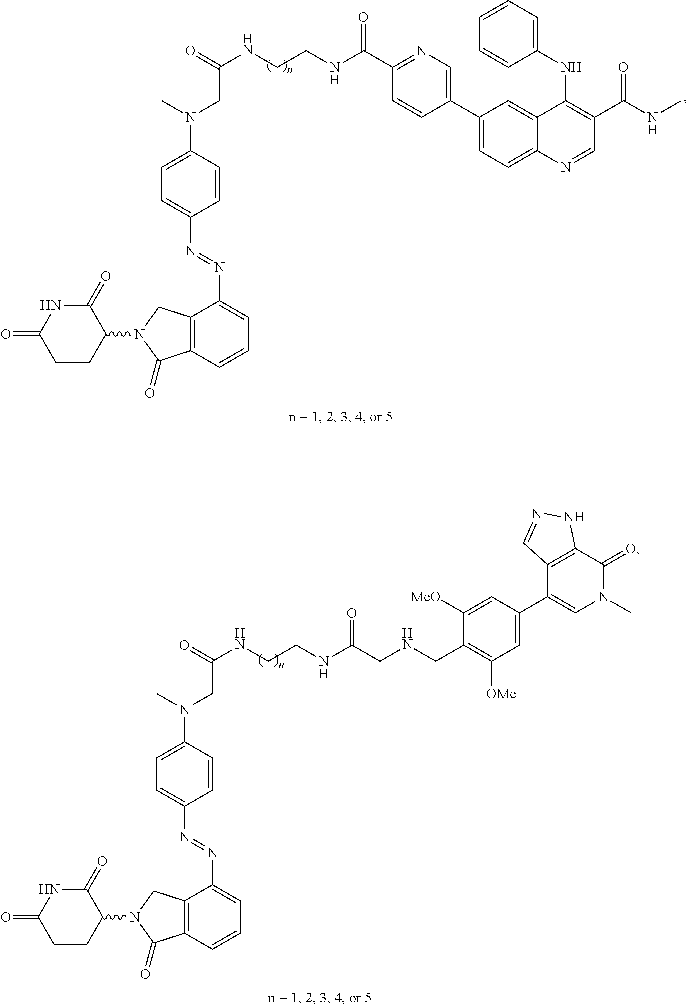

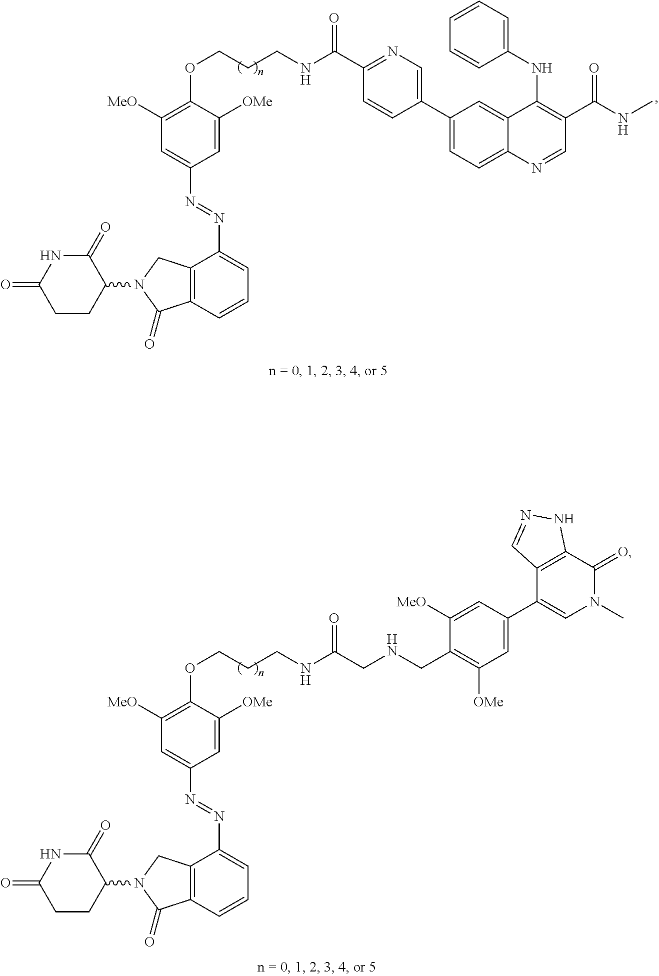

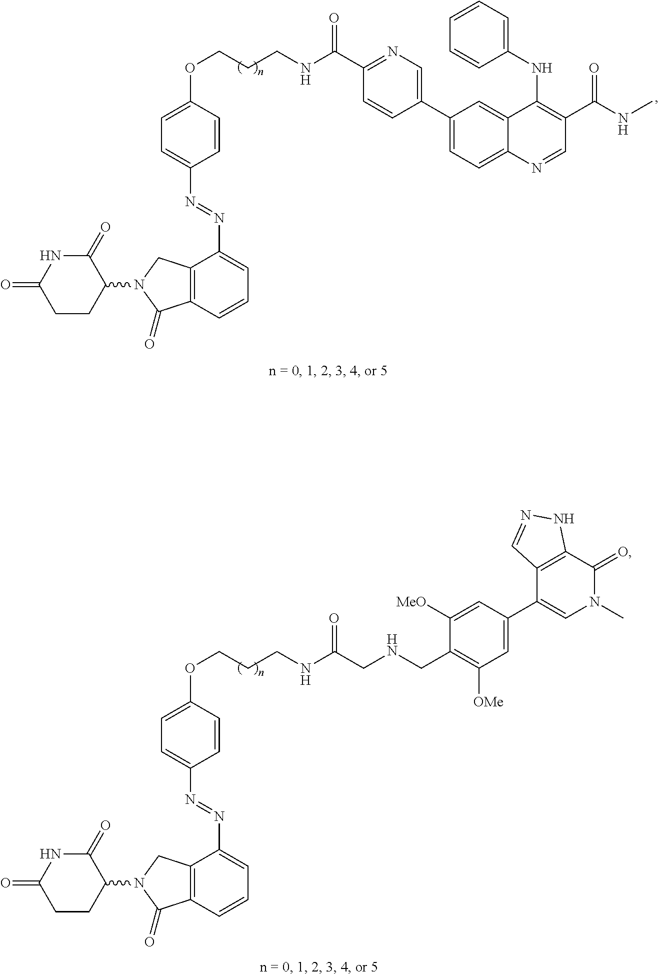

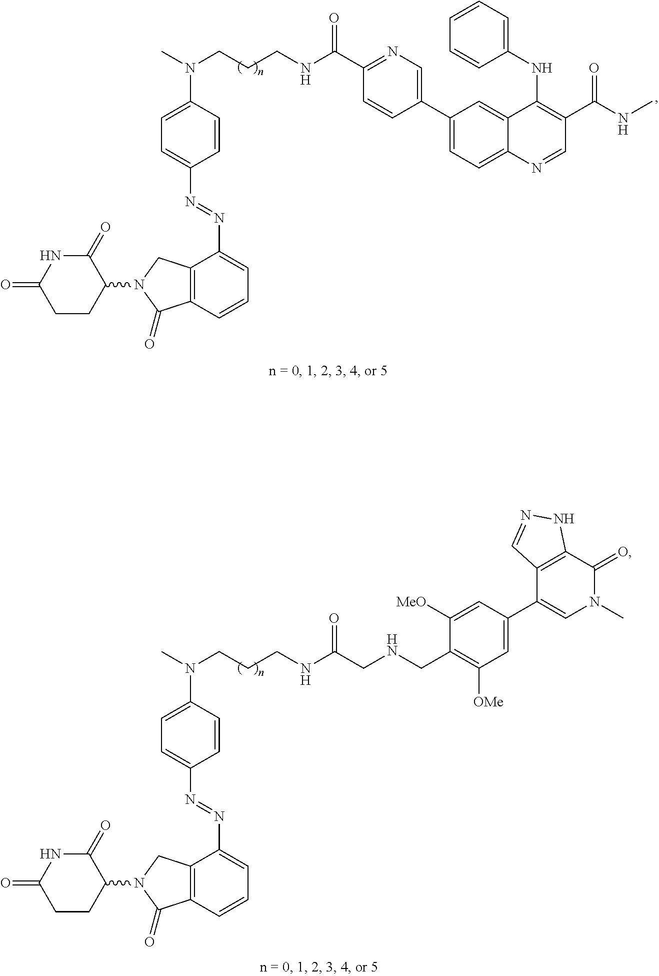

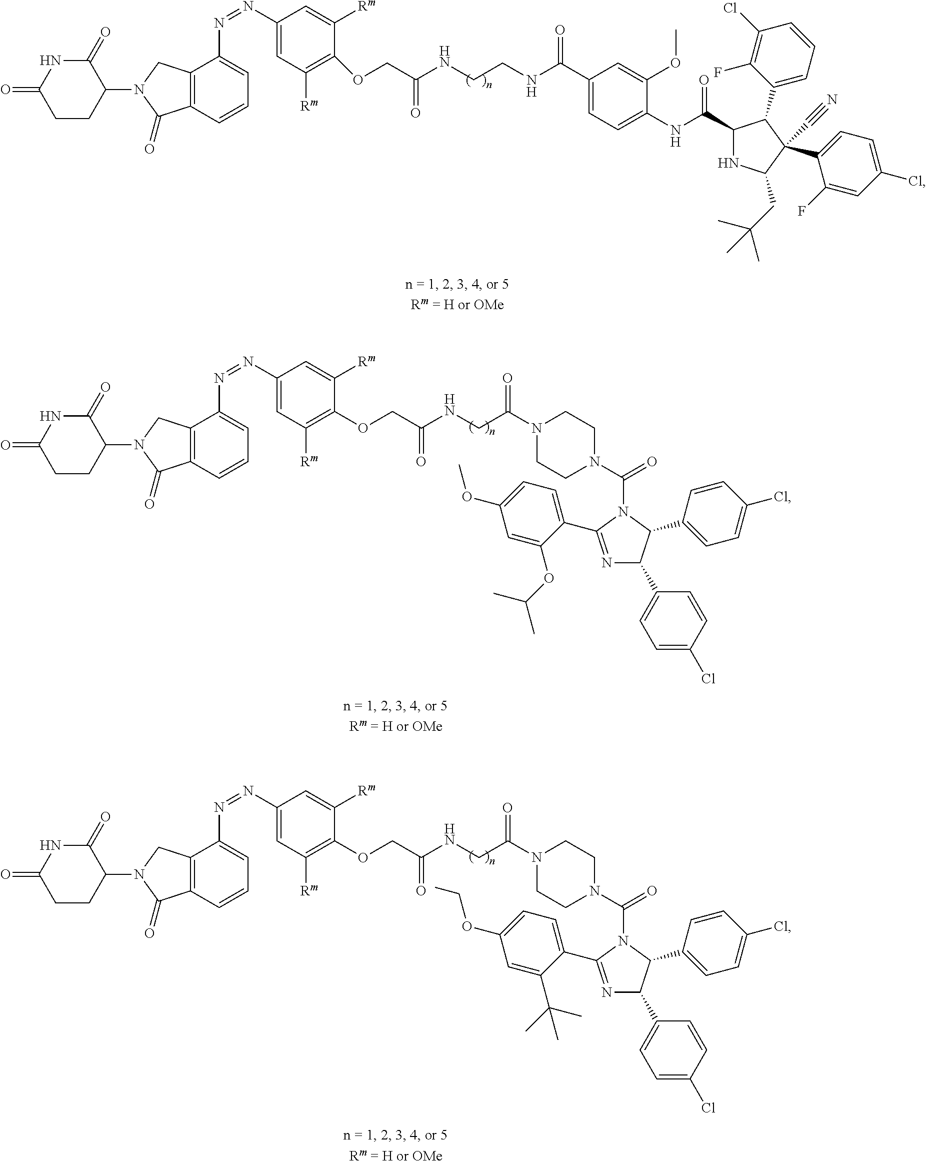

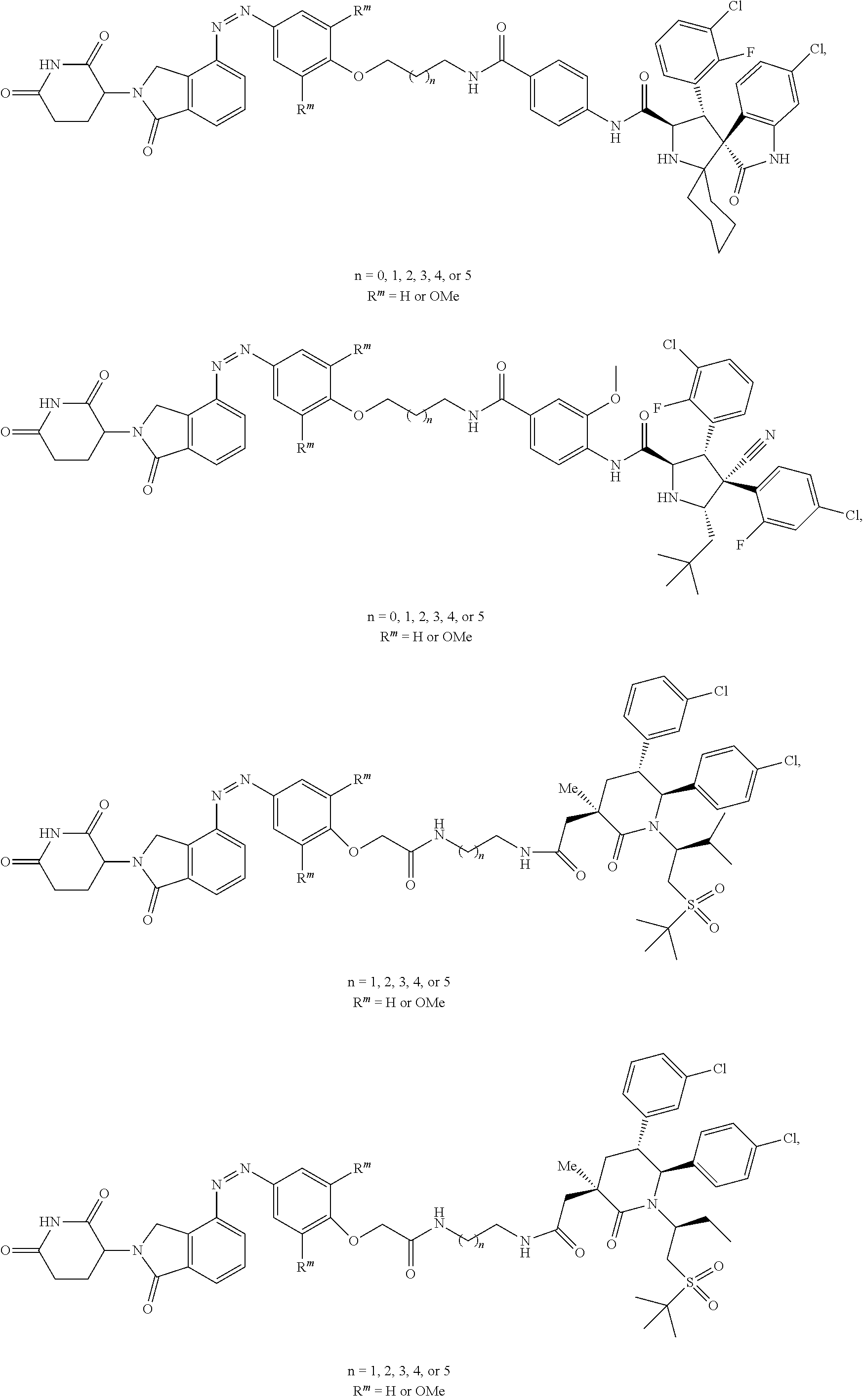

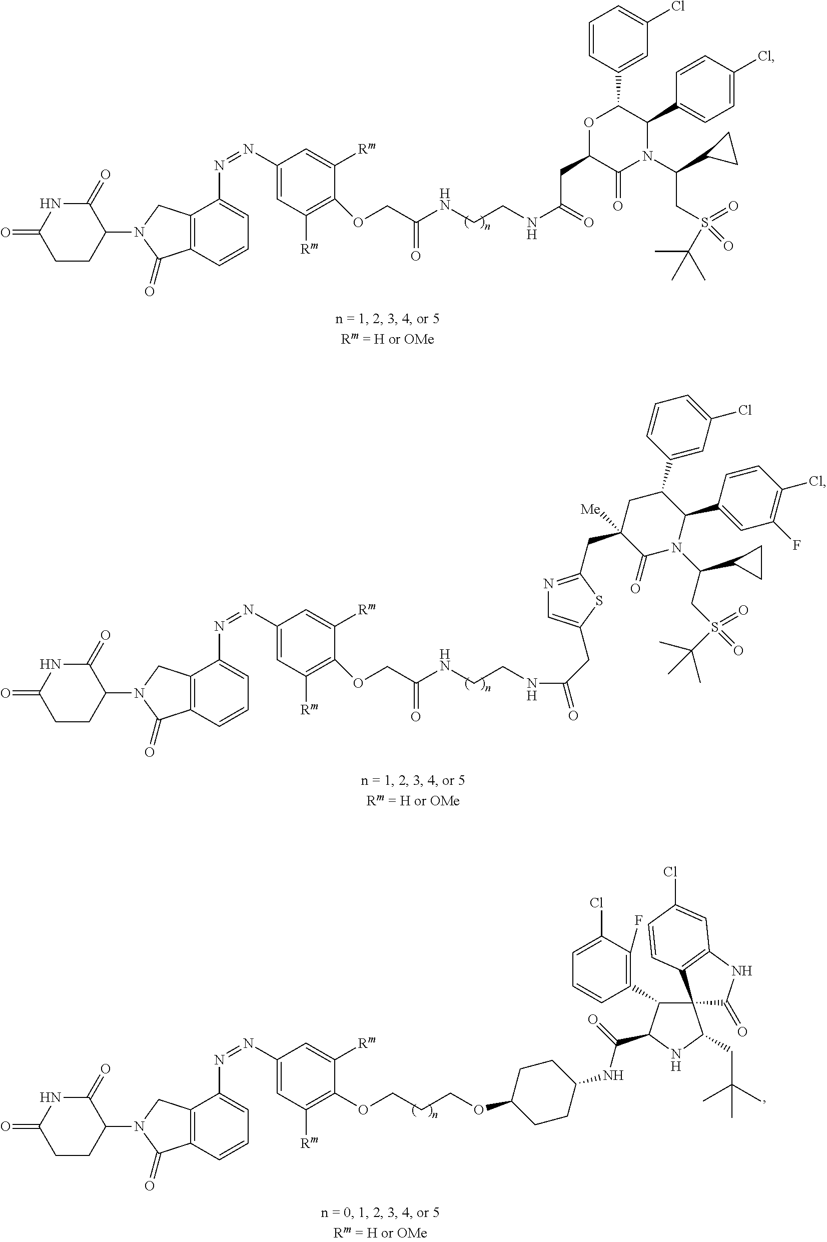

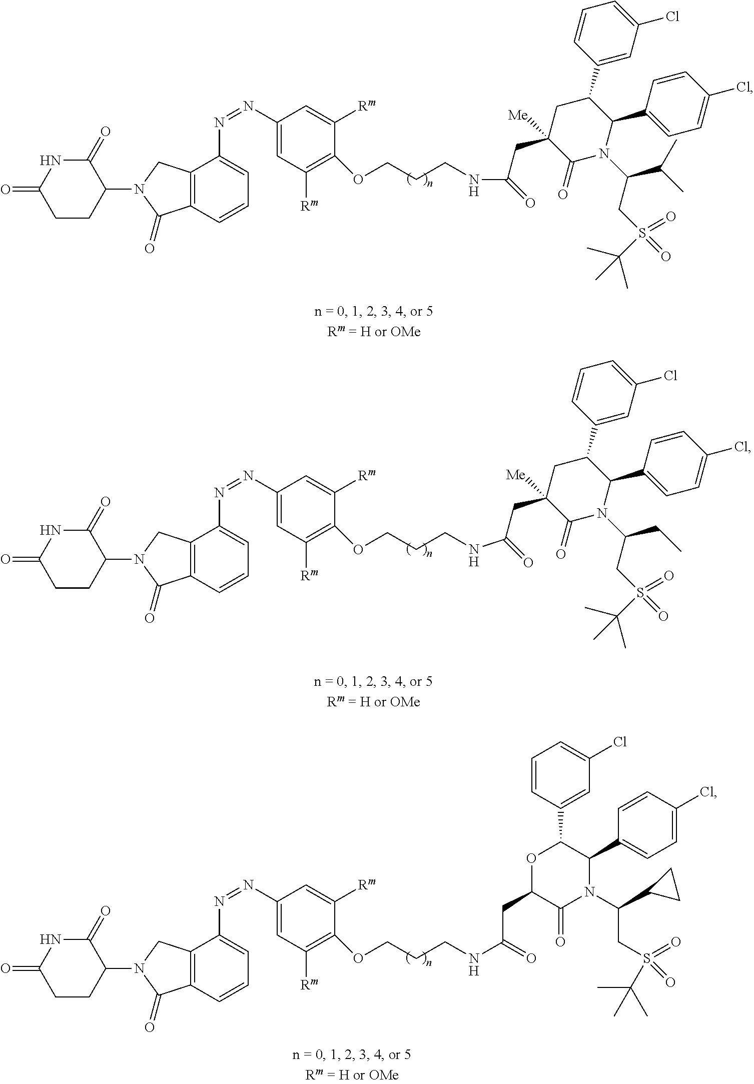

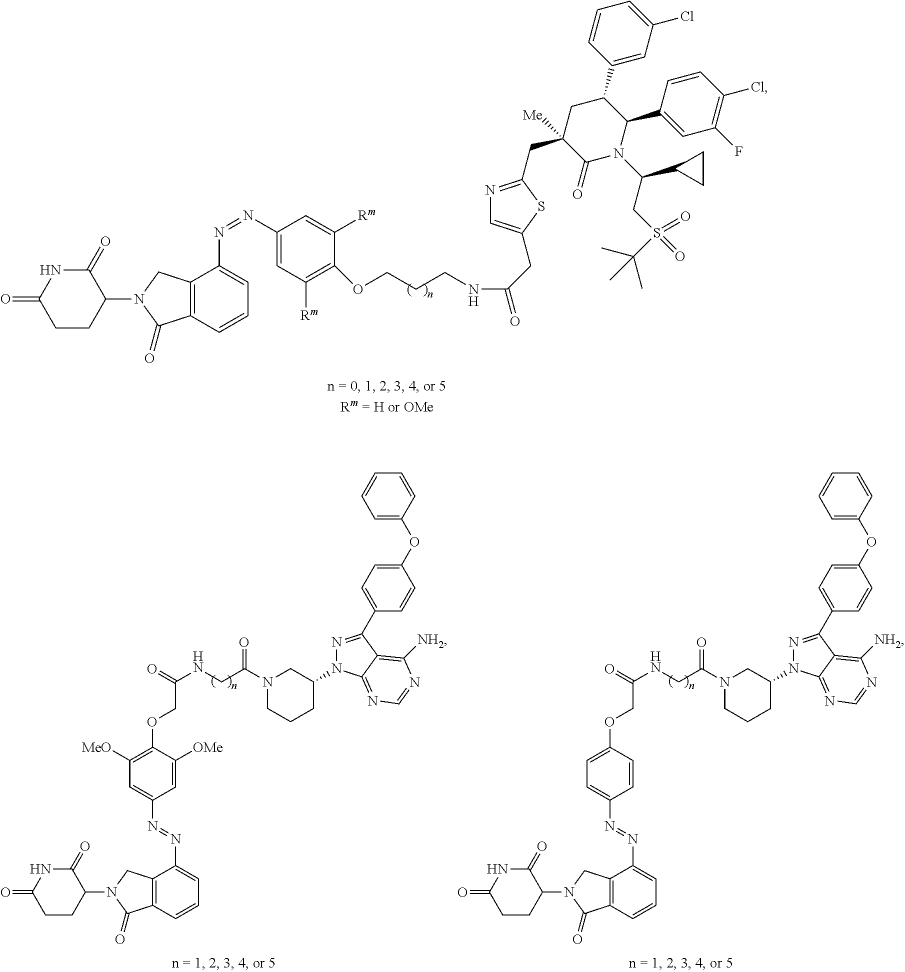

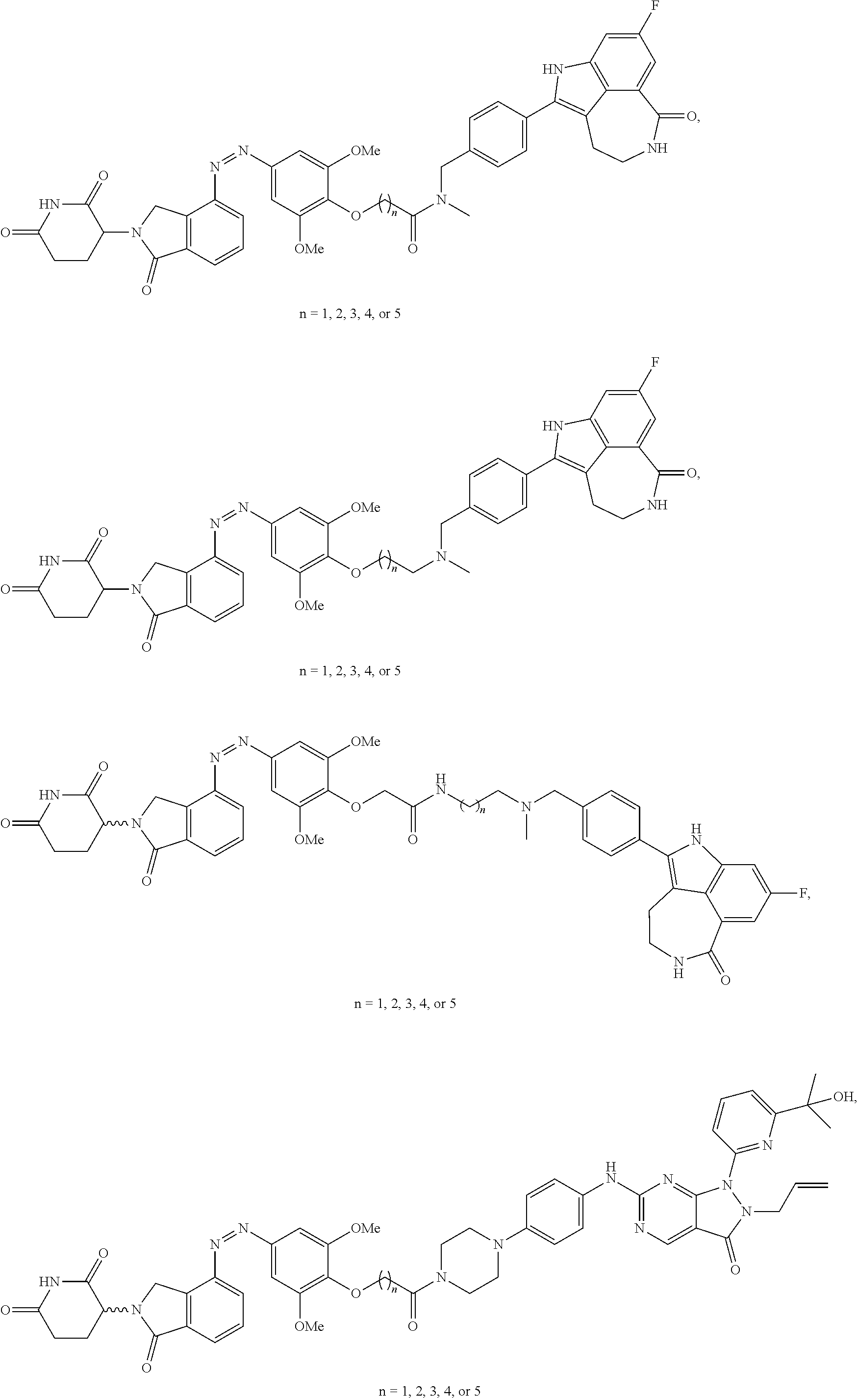

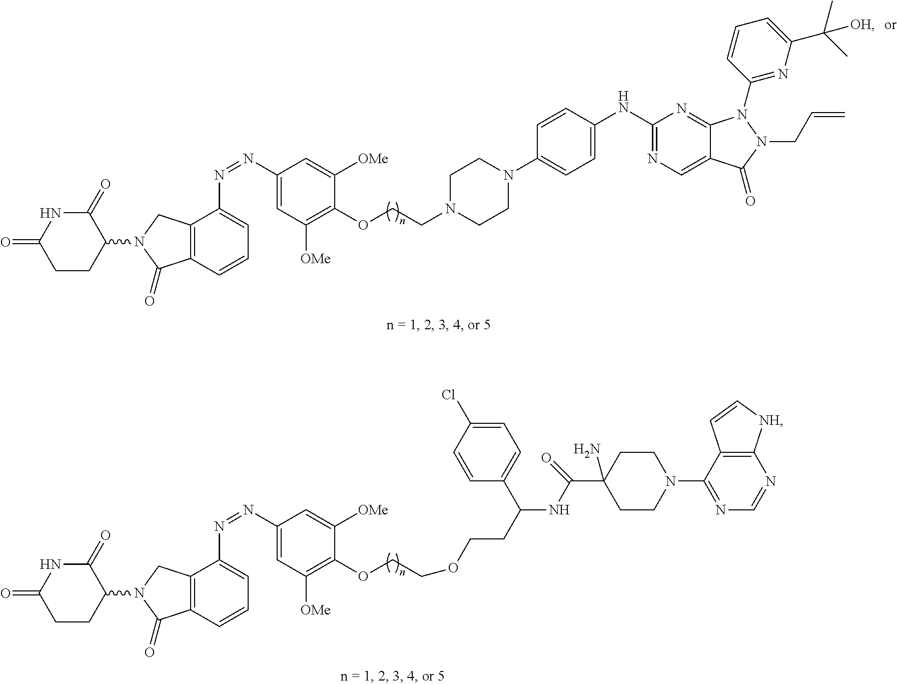

11. A composition comprising a compound of claim 1 and a pharmaceutically acceptable carrier.

12. A method of inducing selective degradation of a target protein in a cell, comprising:
contacting a cell with a compound of claim 1, wherein the compound is in a deactivated conformation and the compound binds to an E3 ligase; and
exposing the cell or a portion thereof to electromagnetic radiation, wherein the exposing induces a conformational change in the compound and the compound binds to the target protein.

13. A method of inducing selective degradation of a target protein in a cell of claim 12, wherein the method further comprises exposing the cell to electromagnetic radiation one or more additional time(s).

14. A method of inducing selective degradation of a target protein in a cell, comprising:
contacting a cell with a compound of claim 1, wherein the compound is in a deactivated conformation and the compound binds to the target protein; and
exposing the cell or a portion thereof to electromagnetic radiation, wherein the exposing induces a conformational change in the compound and the compound binds to an E3 ligase.

15. A method of inducing selective degradation of a target protein in a cell of claim 14, wherein the method further comprises exposing the cell to electromagnetic radiation one or more additional time(s).

16. A method of inducing selective degradation of a target protein in a cell, comprising:
contacting a cell with a compound of claim 1, wherein the compound is in an activated conformation and the compound binds to the target protein and an E3 ligase; and
optionally, exposing the cell or a portion thereof to electromagnetic radiation, wherein the exposing induces a conformational change in the compound.

17. A method of inducing selective degradation of a target protein in a cell of claim 16, wherein the method further comprises exposing the cell to electromagnetic radiation one or more additional time(s).

18. A method of inducing selective degradation of a target protein in a cell, comprising:
contacting a cell with a compound of claim 1, wherein the compound is in a deactivated conformation and the compound binds to the target protein;
waiting a period of time such that the compound relaxes to an activated state and binds to an E3 ligase.

19. A method of inducing selective degradation of a target protein in a cell, comprising:
contacting a cell with a compound of claim 1, wherein the compound is in a deactivated conformation and the compound binds to an E3 ligase;
waiting a period of time such that the compound relaxes to an activated state and binds to the target protein.

20. A method of inducing selective degradation of a target protein in a cell, comprising:
contacting a cell with a compound of claim 1, wherein the compound is in an activated conformation, and the compound binds to the target protein and an E3 ligase;
waiting a period of time such that the compound relaxes to a deactivated state.

21. A method of treating a disease, comprising:
administering to a subject in need of treatment a composition of claim 11, wherein the compound is in a deactivated conformation and the compound binds to an E3 ligase; and
exposing the subject in need of treatment or a portion thereof to electromagnetic radiation, wherein the exposing induces a conformational change in the compound and the compound binds to a target protein.

22. The method of claim 21, further comprising exposing the cells to electromagnetic radiation one or more additional time(s).

23. The method of claim 21, wherein the disease is a cancer and the cancer is chosen from leukemia, lung cancer, dermatological cancers, premalignant lesions of the upper digestive tract, malignancies of prostate, brain, breast, and combinations thereof.

24. The method of claim 21, wherein the disease is chosen from infectious diseases, inflammatory diseases, immune disorders, sleep disorders, neurodegenerative disorders, and combinations thereof.

25. A method of treating a disease, comprising:
administering to a subject in need of treatment a composition of claim 11, wherein the compound is in a deactivated conformation and the compound binds to a target protein; and
exposing the subject in need of treatment or a portion thereof to electromagnetic radiation, wherein the exposing induces a conformational change in the compound and the compound binds to an E3 ligase.

26. The method of claim 25, further comprising exposing the subject in need of treatment to electromagnetic radiation one or more time(s).

27. The method of claim 25, wherein the disease is a cancer and the cancer is chosen from leukemia, lung cancer, dermatological cancers, premalignant lesions of the upper digestive tract, malignancies of prostate, brain, breast, and combinations thereof.

28. The method of claim 25, wherein the disease is chosen from infectious diseases, inflammatory diseases, immune disorders, sleep disorders, neurodegenerative disorders, and combinations thereof.

29. A method of treating a disease, comprising:
administering to a subject in need of treatment a composition of claim 11, wherein the compound is in an activated conformation and the compound binds to a target protein and an E3 ligase; and
optionally, exposing the cell or a portion thereof to electromagnetic radiation; wherein the exposing induces a conformational change in the compound.

30. The method of claim 29, further comprising exposing the cell to electromagnetic radiation one or more additional time(s).

31. The method of claim 29, wherein the disease is a cancer and the cancer is chosen from leukemia, lung cancer, dermatological cancers, premalignant lesions of the upper digestive tract, malignancies of prostate, brain, breast, and combinations thereof.

32. The method of claim 29, wherein the disease is chosen from infectious diseases, inflammatory diseases, immune disorders, sleep disorders, neurodegenerative disorders, and combinations thereof.

33. A method of treating a disease, comprising:
administering to a subject in need of treatment a composition of claim 11, wherein the compound is in a deactivated conformation and the compound binds to the target protein;
waiting a period of time such that the compound relaxes to an activated state and binds to an E3 ligase.

34. The method of claim 33, further comprising exposing the subject in need of treatment to electromagnetic radiation one or more time(s).

35. The method of claim 33, wherein the disease is a cancer and the cancer is chosen from leukemia, lung cancer, dermatological cancers, premalignant lesions of the upper digestive tract, malignancies of prostate, brain, breast, and combinations thereof.

36. The method of claim 33, wherein the disease is chosen from infectious diseases, inflammatory diseases, immune disorders, sleep disorders, neurodegenerative disorders, and combinations thereof.

37. A method of treating a disease, comprising:
administering to a subject in need of treatment a composition of claim 11, wherein the compound is in a deactivated conformation and the compound binds to an E3 ligase;
waiting a period of time such that the compound relaxes to an activated state and binds to the target protein.

38. The method of claim 37, further comprising exposing the subject in need of treatment to electromagnetic radiation one or more time(s).

39. The method of claim 37, wherein the disease is a cancer and the cancer is chosen from leukemia, lung cancer, dermatological cancers, premalignant lesions of the upper digestive tract, malignancies of prostate, brain, breast, and combinations thereof.

40. The method of claim 37, wherein the disease is chosen from infectious diseases, inflammatory diseases, immune disorders, sleep disorders, neurodegenerative disorders, and combinations thereof.

41. A method of treating a disease, comprising:
administering to a subject in need of treatment a composition of claim 11, wherein the compound is in an activated conformation and such that the compound binds to a target protein and an E3 ligase;
waiting a period of time such that the compound relaxes to a deactivated state.

42. The method of claim 41, further comprising exposing the subject in need of treatment to electromagnetic radiation one or more time(s).

43. The method of claim 41, wherein the disease is a cancer and the cancer is chosen from leukemia, lung cancer, dermatological cancers, premalignant lesions of the upper digestive tract, malignancies of prostate, brain, breast, and combinations thereof.

44. The method of claim 41, wherein the disease is chosen from infectious diseases, inflammatory diseases, immune disorders, sleep disorders, neurodegenerative disorders, and combinations thereof.
